(19) United States
(12) Patent Application Publication
(10) Pub. No.: US 2024/0059789 A1
(43) Pub. Date: Feb. 22, 2024

(54) PSMA BINDING PROTEINS AND USES THEREOF

(71) Applicant: Janssen Biotech, Inc., Horsham, PA (US)

(72) Inventors: Theresa McDevitt, Warminster, PA (US); Danlin Dan Qing Yang, Fairfield, CT (US); Sanjaya Singh, Blue Bell, PA (US); Scott Ronald Brodeur, New Hope, PA (US); Jennifer Hertzog, Newton Square, PA (US)

(21) Appl. No.: 18/274,521

(22) PCT Filed: Jan. 24, 2022
(86) PCT No.: PCT/IB2022/050589

§ 371 Date: Jul. 27, 2023

Related U.S. Application Data
(60) Provisional application No. 63/142,921, filed on Jan. 28, 2021.
(60) Provisional application No. 63/165,448, filed on Mar. 24, 2021.

(51) Int. Cl. C07K 16/30; A61P 35/00; C07K 16/28

(52) U.S. Cl. CPC C07K 16/3069; A61P 35/00; C07K 16/2809; C07K 2317/31; C07K 2317/35; A61K 39/00

(57) ABSTRACT

Provided herein, in certain aspects, are antibodies that bind to PSMA, as well as recombinant cells containing the vectors, and compositions comprising the antibodies. Also provided herein, in certain aspects, are multispecific antibodies that bind to PSMA and CD3, as well as recombinant cells containing the vectors, and compositions comprising the antibodies. Methods of making and using the antibodies are also provided.

### CROSS-REFERENCE TO RELATED APPLICATIONS

This application claims the benefit of U.S. Ser. No. 63/142,921 filed Jan. 28, 2021; and U.S. Ser. No. 63/165,448 filed Mar. 24, 2021, the disclosure of each of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file “14620-625-228 SL.txt” and a creation date of Jan. 7, 2022 and having a size of 561,620 bytes. The sequence listing submitted via EFS-Web is part of the specification and is herein incorporated by reference in its entirety.

### FIELD

Provided herein, in certain aspects, are antibodies that bind to prostate-specific membrane antigen (PSMA), as well as recombinant cells containing the vectors, and compositions comprising the antibodies. Methods of making and using the antibodies are also provided. Also provided herein are, in certain aspects, are multispecific antibodies that bind PSMA, as well as recombinant cells containing the vectors, and compositions comprising the antibodies. In some embodiments, the multispecific antibodies bind to PSMA and cluster of differentiation 3 (CD3).

### SUMMARY

In one aspect, provided herein is an isolated antibody that binds PSMA. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the PSMA antibody comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:814.

In some embodiments, the VH CDR1, VH CDR2, VH CDR3, VL CDR1, VL CDR2, and VL CDR3 amino acid sequences of the PSMA antibody are according to the Kabat numbering system. In some embodiments, the VH CDR1, VH CDR2, VH CDR3, VL CDR1, VL CDR2, and VL CDR3 amino acid sequences of the PSMA antibody are according to the Chothia numbering system. In some embodiments, the VH CDR1, VH CDR2, VH CDR3, VL CDR1, VL CDR2, and VL CDR3 amino acid sequences of the PSMA antibody are according to the AbM numbering system. In some embodiments, the VH CDR1, VH CDR2, VH CDR3, VL CDR1, VL CDR2, and VL CDR3 amino acid sequences of the PSMA antibody are according to the Contact numbering system. In some embodiments, the VH CDR1, VH CDR2, VH CDR3, VL CDR1, VL CDR2, and VL CDR3 amino acid sequences of the PSMA antibody are according to the IMGT numbering system. In some embodiments, the VH CDR1, VH CDR2, VH CDR3, VL CDR1, VL CDR2, and VL CDR3 amino acid sequences of the PSMA antibody are according to a combination of the numbering systems provided herein.

In some embodiments, the isolated antibody comprises, consists of and/or consists essentially of a VH having an amino acid sequence at least 80% identical to the amino acid sequence of SEQ ID NOs:31, 99, 167, 235, 303, 371, 405, 439, 473, 507, 541, 575, 643, 677, 711, 779 or 813 and a VL having an amino acid sequence at least 80% identical to the amino acid sequence of SEQ ID NOs:32, 66, 100, 134, 236, 270, 372, 406, 474, 508, 542, 576, 678, 746, 780 or 814.

In some embodiments, the isolated antibody comprises, consists of and/or consists essentially of a heavy chain (HC) having an amino acid sequence at least 80% identical to the amino acid sequence of SEQ ID NOs:33, 101, 169, 237, 305, 373, 407 or 441 and a light chain (LC) having an amino acid sequence at least 80% identical to the amino acid sequence of SEQ ID NOs:34, 68, 102, 136, 238, 272, 374, or 408.

In some embodiments, provided herein is an isolated antibody that binds PSMA comprising, consisting of and/or consisting essentially of (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In some embodiments, provided herein is an isolated antibody that binds PSMA comprising, consisting of and/or consisting essentially of (i) a VH comprising, consisting of and/or consisting essentially of a VH CDR1, a VH CDR2, and a VH CDR3 having an amino acid sequence of SEQ ID NOs:205, 206, and 411, respectively, and (ii) a VL comprising, consisting of and/or consisting essentially of a VL CDR1, VL CDR2, and VL CDR3 having an amino acid sequence of SEQ ID NOs:242, 209, and 244, respectively. In some embodiments, provided herein is an isolated antibody that binds PSMA comprising, consisting of and/or consisting essentially of (i) a VH having an amino acid sequence of SEQ ID NO:439; and (ii) a VL having an amino acid sequence of SEQ ID NO:270. In some embodiments, provided herein is an isolated antibody that binds PSMA comprising, consisting of and/or consisting essentially of (i) a HC having an amino acid sequence of SEQ ID NO:441; and (ii) a LC having an amino acid sequence of SEQ ID NO:272.

In some embodiments, the isolated antibody binds a PSMA antigen. In some embodiments, isolated antibody binds a PSMA epitope. In some embodiments, the isolated antibody specifically binds to PSMA. In some embodiments, the VH CDR1, VH CDR2, VH CDR3, VL CDR1, VL CDR2 and VL CDR3 form a binding site for an antigen of the PSMA. In some embodiments, the VH CDR1, VH CDR2, VH CDR3, VL CDR1, VL CDR2 and VL CDR3 form a binding site for an epitope of the PSMA. In some embodiments, the PSMA is present on the surface of a cell. In some embodiments, the PSMA is present on the surface of a prostate cell. In some embodiments, the PSMA is present on the surface of a prostate cancer cell. In some embodiments, the PSMA is present on the surface of a renal cell. In some embodiments, the PSMA is present on the surface of a renal cancer cell.

In some embodiments of the PSMA antibody provided herein the binding domain that binds to PSMA is a scFv, an scFv dimer, a Fv, a Fab, a Fab, a F(ab)₂, a dsFv, a sdAb, a VHH or a single chain antibody.

In some embodiments, the PSMA antibody is a humanized antibody. In some embodiments, the PSMA antibody is a human antibody. In some embodiments, the isolated antibody is an IgG antibody. In some embodiments, the PSMA antibody is an IgG1 antibody. In some embodiments, the PSMA antibody is an IgG2 antibody. In some embodiments, the PSMA antibody is an IgG3 antibody. In some embodiments, the PSMA antibody is an IgG4 antibody. In some embodiments, the PSMA antibody comprises, consists of and/or consists essentially of a kappa light chain. In some embodiments, the PSMA antibody comprises, consists of and/or consists essentially of a lambda light chain. In some embodiments, the PSMA antibody is a monoclonal antibody. In some embodiments, the PSMA antibody is multivalent. In some embodiments, the PSMA antibody is capable of binding at least three antigens. In some embodiments, the PSMA antibody is capable of binding at least four antigens. In some embodiments, the PSMA antibody is capable of binding at least five antigens. In some embodiments, the PSMA antibody is a multispecific antibody. In some embodiments, the PSMA antibody is a bispecific antibody. In some embodiments, the PSMA antibody is a trispecific antibody. In some embodiments, the PSMA antibody is a quadraspecific antibody.

In another aspect, provided is a nucleic acid encoding a PSMA antibody provided herein. Also provided is a vector comprising, consisting of and/or consisting essentially of a nucleic acid encoding a PSMA antibody provided herein. Also provided is a host cell comprising, consisting of and/or consisting essentially of a vector comprising, consisting of and/or consisting essentially of a nucleic acid encoding a PSMA antibody provided herein. Also provided is a kit comprising, consisting of and/or consisting essentially of vector comprising, consisting of and/or consisting essentially of a nucleic acid encoding a PSMA antibody provided herein, and packaging for the same.

In another aspect, provided is a kit comprising, consisting of and/or consisting essentially of a PSMA antibody provided herein, and packaging for the same.

In another aspect, provided is a pharmaceutical composition comprising, consisting of and/or consisting essentially of a PSMA antibody provided herein, and a pharmaceutically acceptable carrier.

In another aspect, provided is a method of producing a pharmaceutical composition comprising, consisting of and/or consisting essentially of a PSMA antibody provided herein, comprising, consisting of and/or consisting essentially of combining the PSMA antibody with a pharmaceutically acceptable carrier to obtain the pharmaceutical composition.

In another aspect, provided is an isolated multispecific PSMAxCD3 antibody, comprising, consisting of and/or consisting essentially of: (a) a first binding domain that binds to PSMA, and (b) a second binding domain that binds CD3. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In some embodiments, the first binding domain that binds to PSMA comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In some embodiments, the second binding domain that binds to CD3 comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:1505; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:1464. In some embodiments, the second binding domain that binds to CD3 comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In some embodiments, the second binding domain that binds to CD3 comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In some embodiments, the second binding domain that binds to CD3 comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In some embodiments, the second binding domain that binds to CD3 comprises, consists of and/or consists essentially of: (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:1463; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:1464. In some embodiments, the second binding domain that binds to CD3 comprises, consists of and/or consists essentially of a scFv comprising, consisting of and/or consisting essentially of a VH CDR1, a VH CDR2, a VH CDR3, a VL CDR1, a VL CDR2, a VL CDR3 having an amino acid sequence of a VH CDR1, a VH CDR2, a VH CDR3, a VL CDR1, a VL CDR2, a VL CDR3 respectively, of a scFv having an amino acid sequence of SEQ ID NO:1524.

In some embodiments, the VH CDR1, VH CDR2, VH CDR3, VL CDR1, VL CDR2, and VL CDR3 amino acid sequences of the multispecific PSMAxCD3 antibody are according to the Kabat numbering system. In some embodiments, the VH CDR1, VH CDR2, VH CDR3, VL CDR1, VL CDR2, and VL CDR3 amino acid sequences of the multispecific PSMAxCD3 antibody are according to the Chothia numbering system. In some embodiments, the VH CDR1, VH CDR2, VH CDR3, VL CDR1, VL CDR2, and VL CDR3 amino acid sequences of the multispecific PSMAxCD3 antibody are according to the AbM numbering system. In some embodiments, the VH CDR1, VH CDR2, VH CDR3, VL CDR1, VL CDR2, and VL CDR3 amino acid sequences of the multispecific PSMAxCD3 antibody are according to the Contact numbering system. In some embodiments, the VH CDR1, VH CDR2, VH CDR3, VL CDR1, VL CDR2, and VL CDR3 amino acid sequences of the multispecific PSMAxCD3 antibody are according to the IMGT numbering system. In some embodiments, the VH CDR1, VH CDR2, VH CDR3, VL CDR1, VL CDR2, and VL CDR3 amino acid sequences of the multispecific PSMAxCD3 antibody are according to a combination of the numbering systems provided herein.

In some embodiments of the multispecific PSMAxCD3 antibody provided herein, the first binding domain that binds PSMA comprises, consists of and/or consists essentially of a heavy chain (HC) having an amino acid sequence at least 80% identical to the amino acid sequence of SEQ ID NOs:33, 101, 169, 237, 305, 373, 407, 441, 1242, 1244, 1248, 1250, 1252, 1254, 1256, 1258, 1260, 1262, 1264, 1266, 1268 or 1270 and/or a light chain (LC) having an amino acid sequence at least 80% identical to the amino acid sequence of SEQ ID NOs:34, 68, 102, 136, 238, 272, 374 or 408.

In some embodiments of the multispecific PSMAxCD3 antibody provided herein, the first binding domain that binds PSMA comprises, consists of and/or consists essentially of a scFv having an amino acid sequence at least 80% identical to the amino acid sequence of any one of SEQ ID NOs:1485-1500 or SEQ ID NOs:1526-1531.

In some embodiments of the multispecific PSMAxCD3 antibody provided herein, the first binding domain that binds PSMA comprises, consists of and/or consists essentially of (i) a HC having an amino acid sequence of SEQ ID NO:441; and (ii) a LC having an amino acid sequence of SEQ ID NO:272.

In some embodiments of the multispecific PSMAxCD3 antibody provided herein, the second binding domain that binds CD3 comprises, consists of and/or consists essentially of a heavy chain (HC) having an amino acid sequence at least 80% identical to the amino acid sequence of SEQ ID NOs:849, 883, 917, 951, 985, 1019, 1504, 1455, 1192, 1194, 1167, 1218 or 1238 and/or a light chain (LC) having an amino acid sequence at least 80% identical to the amino acid sequence of SEQ ID NOs:850, 918, 986, 1193, 1195 or 1219.

In some embodiments of the multispecific PSMAxCD3 antibody provided herein, the second binding domain that binds CD3 comprises, consists of and/or consists essentially of a scFv having an amino acid sequence at least 80% identical to the amino acid sequence of SEQ ID NOs:1186, 1187, 1523 or 1524.

Provided herein is an isolated bispecific antibody comprising, consisting of and/or consisting essentially of a first binding domain that binds PSMA and a second binding domain that binds CD3, wherein the first binding domain that binds PSMA comprises, consists of and/or consists essentially of (i) a VH comprising, consisting of and/or consisting essentially of a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising, consisting of and/or consisting essentially of a VL CDR1, a VL CDR2, and a VL CDR3 having an amino acid sequence of a VL CDR1, a VL CDR2, and a VL CDR3, respectively, of a VL having an amino acid sequence of SEQ ID NO:270, and the second binding domain that binds CD3 comprises, consists of and/or consists essentially of a scFv comprising, consisting of and/or consisting essentially of a VH CDR1, a VH CDR2, a VH CDR3, a VL CDR1, a VL CDR2, and a VL CDR3 of a scFv having an amino acid sequence of SEQ ID NO:1524. In some embodiments of the multispecific PSMAxCD3 antibody provided herein, the first binding domain that binds PSMA comprises, consists of and/or consists essentially of a VH CDR1, VH CDR2, VH CDR3, VL CDR1, VL CDR2, VL CDR3 of SEQ ID NO:205, 206, 411, 242, 209 and 244, respectively, wherein the amino acid sequences are according to the Kabat numbering system; and the second binding domain that binds CD3 comprises, consists of and/or consists essentially of a VH CDR1, VH CDR2, VH CDR3, VL CDR1, VL CDR2, VL CDR3 of SEQ ID NO:1467, 1468, 1506, 1470, 1471 and 1472, respectively; wherein the amino acid sequences are according to the Kabat numbering system. In some embodiments of the multispecific PSMAxCD3 antibody provided herein, the first binding domain that binds PSMA comprises, consists of and/or consists essentially of (i) a VH having an amino acid sequence of SEQ ID NO:439; and (ii) a VL having an amino acid sequence of SEQ ID NO:270, and the second binding domain that binds CD3 comprises, consists of and/or consists essentially of a scFv of SEQ ID NO:1524, respectively. In some embodiments of the multispecific PSMAxCD3 antibody provided herein, the first binding domain that binds PSMA comprises, consists of and/or consists essentially of (i) a HC2 having an amino acid sequence of SEQ ID NO:441; and (ii) a LC2 having an amino acid sequence of SEQ ID NO:272, respectively; and the second binding domain that binds CD3 comprises, consists of and/or consists essentially of a HC1 of SEQ ID NO:1455.

In some embodiments of the multispecific antibody provided herein, the first binding domain, the second binding domain and/or the first and second is a scFv, an scFv dimer, a Fv, a Fab, a Fab, a F(ab′)2, a dsFv, a sdAb, a VHH or a single chain antibody.

In some embodiments, the multispecific PSMAxCD3 antibody is a humanized antibody. In some embodiments, the multispecific PSMAxCD3 antibody is a human antibody. In some embodiments, the multispecific PSMAxCD3 antibody is an IgG antibody. In some embodiments, the multispecific PSMAxCD3 antibody is an IgG1 antibody. In some embodiments, the multispecific PSMAxCD3 antibody is an IgG2 antibody. In some embodiments, the multispecific PSMAxCD3 antibody is an IgG3 antibody. In some embodiments, the multispecific PSMAxCD3 antibody is an IgG4 antibody. In some embodiments, the multispecific PSMAxCD3 antibody comprises, consists of and/or consists essentially of a kappa light chain. In some embodiments, the multispecific PSMAxCD3 antibody comprises, consists of and/or consists essentially of a lambda light chain. In some embodiments, the multispecific PSMAxCD3 antibody is a monoclonal antibody.

In some embodiments of the multispecific PSMAxCD3 antibody, the first binding domain binds a PSMA antigen. In some embodiments of the multispecific PSMAxCD3 antibody, the first binding domain binds a PSMA epitope. In some embodiments of the multispecific PSMAxCD3 antibody, the first binding domain specifically binds to PSMA. In some embodiments of the multispecific PSMAxCD3 antibody, the VH CDR1, VH CDR2, VH CDR3, VL CDR1, VL CDR2 and VL CDR3 of the first binding domain form a binding site for an antigen of the PSMA. In some embodiments of the multispecific PSMAxCD3 antibody, the VH CDR1, VH CDR2, VH CDR3, VL CDR1, VL CDR2 and VL CDR3 of the first binding domain form a binding site for an epitope of the PSMA. In some embodiments of the multispecific PSMAxCD3 antibody, the second binding domain binds a CD3 antigen. In some embodiments of the multispecific PSMAxCD3 antibody, the second binding domain binds a CD3 epitope. In some embodiments of the multispecific PSMAxCD3 antibody, the second binding domain specifically binds to CD3. In some embodiments of the multispecific PSMAxCD3 antibody, the second binding domain form a binding site for an antigen of the CD3. In some embodiments of the multispecific PSMAxCD3 antibody, the VH CDR1, VH CDR2, VH CDR3, VL CDR1, VL CDR2 and VL CDR3 of the multispecific PSMAxCD3 antibody binding domain form a binding site for an epitope of the CD3.

In some embodiments, the PSMA is present on the surface of a cell. In some embodiments, the cell is a prostate cell. In some embodiments, the cell is a prostate cancer cell. In some embodiments, the cell is a renal cell. In some embodiments, the cell is a renal cancer cell.

In some embodiments of the multispecific PSMAxCD3 antibody, the antibody is a bispecific antibody. In some embodiments of the multispecific PSMAxCD3 antibody, is a trispecific antibody. In some embodiments of the multispecific PSMAxCD3 antibody, is a quadraspecific antibody.

In another aspect, provided is a nucleic acid encoding a multispecific PSMAxCD3 antibody provided herein. Also provided is a vector comprising, consisting of and/or consisting essentially of a nucleic acid encoding a multispecific PSMAxCD3 antibody provided herein. Also provided is a host cell comprising, consisting of and/or consisting essentially of a vector comprising, consisting of and/or consisting essentially of a nucleic acid encoding a multispecific PSMAxCD3 antibody provided herein. Also provided is a kit comprising, consisting of and/or consisting essentially of vector comprising, consisting of and/or consisting essentially of a nucleic acid encoding a multispecific PSMAxCD3 antibody provided herein, and packaging for the same.

In another aspect, provided is a kit comprising, consisting of and/or consisting essentially of a multispecific PSMAxCD3 antibody provided herein, and packaging for the same.

In another aspect, provided is a pharmaceutical composition comprising, consisting of and/or consisting essentially of a multispecific PSMAxCD3 antibody provided herein, and a pharmaceutically acceptable carrier.

In another aspect, provided is a method of producing a pharmaceutical composition comprising, consisting of and/or consisting essentially of a multispecific PSMAxCD3 antibody provided herein, comprising, consisting of and/or consisting essentially of combining the multispecific PSMAxCD3 antibody with a pharmaceutically acceptable carrier to obtain the pharmaceutical composition.

In another aspect, provided is a method of directing a CD3-expressing T cell to a PSMA-expressing target cell, comprising, consisting of and/or consisting essentially of contacting the T cell with the multispecific PSMAxCD3 antibody provided herein. In some embodiments, the contacting directs the T cell to the target cell. In another aspect, provided is a method of inhibiting the growth or proliferation of a PSMA-expressing target cell, comprising, consisting of and/or consisting essentially of contacting the target cell with the multispecific PSMAxCD3 antibody provided herein. In some embodiments, the contacting is in the presence of CD3-expressing T cells. In some embodiments, the target cell expresses PSMA on the cell surface. In some embodiments, the target cell is a prostate cell. In some embodiments, the target cell is a prostate cancer cell. In some embodiments, the target cell is a renal cell. In some embodiments, the target cell is a renal cancer cell.

In another aspect, provided is a method of eliminating a PSMA-expressing target cell in a subject, comprising, consisting of and/or consisting essentially of administering to the subject an effective amount of the multispecific PSMAxCD3 antibody provided herein. In another aspect, provided is a method of treating a disease or disorder in a subject, comprising, consisting of and/or consisting essentially of administering to the subject an effective amount of the multispecific PSMAxCD3 antibody provided herein. In some embodiments, the disease or disorder is a disease or disorder of the prostate. In some embodiments, the disease or disorder of the prostate is prostate inflammation. In some embodiments, the disease or disorder of the prostate is Benign prostatic hyperplasia. In some embodiments, the disease or disorder of the prostate is prostate cancer. In some embodiments, the disease or disorder of the prostate is metastatic castration-resistant prostate cancer (mCRPC). In some embodiments, the disease or disorder is a renal disease or disorder. In some embodiments, the renal disease or disorder is renal cancer. In some embodiments, the renal disease or disorder is a renal cell carcinoma. In some embodiments, the renal cell carcinoma is a metastatic renal cell carcinoma (mRCC). In some embodiments, the subject is a subject in need thereof. In some embodiments, the subject is a human.

### BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 shows HDX-MS epitope mapping of PSMA against PS3B1352 (top) and PS3B1353 (bottom). G is glycosylation site. Black box is epitope and gray is probable epitope. White box indicates no/little change in deuteration level in the presence of the antibody. The residues without box indicate the HDX behaviors were not monitored, because there is no peptide to cover the residues or the residues are the first two residues of a peptide. The epitopes of PS3B1352 and PS3B1353 are identical. FIG. 1 discloses SEQ ID NO:1483.

FIG. 2 shows HDX-MS identified epitopes of PSMA overlaid on X-ray crystal structure. The circled alpha-helix represents the HDX-MS identified epitope. The black: epitope; gray: potential epitope.

FIG. 3 shows PAN-T cell binding assay. Human PAN-T cells were treated with various concentrations of PSMA/CD3 bispecific antibodies and incubated at 37° C. for 30 minutes followed by CD3 cell surface expression analysis by flow cytometry.

FIG. 4 shows the non-linear regression fit of four-parameter function of PSMA ligand binding of C4-2B human prostate tumor cells.

FIG. 5 shows a target cell binding assay. C4-2B human prostate tumor cells were treated with various concentrations of PSMA/CD3 bispecific antibodies and incubated at 37° C. for 30 minutes followed by PSMA cell surface expression analysis by flow cytometry.

FIG. 6 shows internalization of PSMA. Human C4-2B prostate tumor cells were incubated with PSMA/CD3 bispecific antibodies conjugated to IncuCyte® Human Fab-fluor-pH Red Antibody Labeling Dye for 24 hours.

FIGS. 7A-7H show bispecific anti-PSMA/anti-T cell redirection antibodies evaluated in an IncuCyte®-based cytotoxicity assay. Isolated PAN-T cells were co-incubated with PSMA+ C4-2B cells in the presence of bispecific PSMA/T cell redirection antibodies for 120 hours. Shown are data for (A) PS3B1352, (B) PS3B1356, (C) PS3B1353, (D) PS3B1357, (E) PS3B1354, (F) PS3B937, (G) PS3B1355, and (H) PS3B1358.

FIG. 8 shows T cell redirected killing assay. Normal human PBMCs were combined with C4-2B human prostate tumor cells transduced with IncuCyte® NucLight red nuclear dye and treated with PSMA/CD3 bispecific antibodies for 5 days.

FIG. 9 shows cytokine induction by bispecific anti-PSMA/anti-T cell redirection antibodies. Isolated PAN-T cells were co-incubated with PSMA+ C4-2B cells in the presence of bispecific anti-PSMA/anti-T cell redirection antibodies for the indicated time points. IFN-gamma concentration was measured from supernatants collected at the indicated time points.

### DETAILED DESCRIPTION

Various publications, articles and patents are cited or described in the background and throughout the specification; each of these references is herein incorporated by reference in its entirety as though fully set forth. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. Although any methods and materials similar or equivalent to those described herein can be used in the practice for testing embodiments of the present disclosure, exemplary materials and methods are described herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

It must be noted that as used herein and in the appended claims, the singular forms “a,” “an,” and “the” include plural reference unless the context clearly dictates otherwise. Thus, for example, reference to “a cell” includes a combination of two or more cells, and the like.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). “About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series.

Those skilled in the art will recognize or be able to ascertain using no more than routine experimentation, many equivalents to the specific embodiments described herein. Such equivalents are intended to been encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, such as a mammal or a human. The term “mammal” as used herein, encompasses any mammal. Examples of mammals include, but are not limited to, cows, horses, sheep, pigs, cats, dogs, mice, rats, rabbits, guinea pigs, monkeys, humans, etc. In specific embodiments, the subject is a human.

It should also be understood that the terms “about,” “approximately,” “generally,” “substantially,” and like terms, used herein when referring to a dimension or characteristic of a component of embodiments provided herein, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., PSMA antibodies and polynucleotides that encode them, CD3 antibodies and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more such as less than about 0.01, and or less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, “variant” refers to a polypeptide or a polynucleotide that differs from a reference polypeptide or a reference polynucleotide by one or more modifications, for example one or more substitutions, insertions or deletions.

As used herein, the term “vector” is a replicon in which another nucleic acid segment can be operably inserted so as to bring about the replication or expression of the segment. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide can be DNA or RNA molecules or a hybrid of these, single stranded or double stranded.

As used herein, “expression vector” refers to a vector that can be utilized in a biological system or in a reconstituted biological system to direct the translation of a polypeptide encoded by a polynucleotide sequence present in the expression vector. As used herein, the term “host cell” refers to a cell comprising a nucleic acid molecule provided herein.

As used herein, “host cell” can be any type of cell, e.g., a primary cell, a cell in culture, or a cell from a cell line. In one embodiment, a “host cell” is a cell transfected with a nucleic acid molecule provided herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, “flow cytometry” is a technology that is used to analyze the physical and chemical characteristics of particles in a fluid as it passes through at least one laser. Cell components are fluorescently labelled and then excited by the laser to emit light at varying wavelengths (Adan et al, Crit. Rev. Biotech. (2016) 1549-7801).

As used herein, “overexpress”, “overexpressed” and “overexpressing” interchangeably refers to a sample such as a cancer cell, malignant cell or cancer tissue that has measurably higher levels of PSMA when compared to a reference sample. The overexpression can be caused by gene amplification or by increased transcription or translation. Expression and overexpression of protein in the sample can be measured using well known assays using, for example ELISA, immunofluorescence, flow cytometry or radioimmunoassay on live or lysed cells. Expression and overexpression of a polynucleotide in the sample can be measured, for example, using fluorescent in situ hybridization, Southern blotting, or PCR techniques. A protein or a polynucleotide is overexpressed when the level of the protein or the polynucleotide in the sample is at least 1.5-fold higher when compared to the reference sample. Selection of the reference sample is well known.

As used herein, “sample” refers to a collection of similar fluids, cells, or tissues isolated from a subject, as well as fluids, cells, or tissues present within a subject. Exemplary samples are of biological fluids such as blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage, liquid solutions contacted with a subject or biological source, for example, cell and organ culture medium including cell or organ conditioned medium, lavage fluids and the like, tissue biopsies, fine needle aspirations or surgically resected tumor tissue.

A “cancer cell” or a “tumor cell” as used herein refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid or uptake of exogenous nucleic acid, it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo (Freshney, Culture of Animal Cells: A Manual of Basic Technique (3rd ed. 1994)). Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

Conventional one and three-letter amino acid codes are used herein as shown in Table 1.

TABLE 1
Conventional Amino Acid Codes

| | Three- | One- |
|---|---|---|
| | letter | letter |
| Amino acid | code | code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, “effector antigens” are antigens from cells of the immune system, which can stimulate or trigger cytotoxicity, phagocytosis, antigen presentation, cytokine release. Such effector antigens are from, for example but not limited to, T cells and natural killer (NK) cells. Examples of suitable specificities for effector antigens include but are not limited to CD3 or CD3 subunits such as CD3E for T cells and CD16 for NK cells. Such cell surface molecules of effector cells are suitable for mediating cell killing. Effector cells are cells of the immune system, which can stimulate or trigger cytotoxicity, phagocytosis, antigen presentation, cytokine release. Such effector cells are, for example but not limited to, T cells, natural killer (NK) cells, granulocytes, monocytes, macrophages, dendritic cells, and antigen-presenting cells. Examples of suitable specificities for effector cells include but are not limited to CD2, CD3 and CD3 subunits such as CD3e, CD5, CD28 and other components of the T cell receptor (TCR) for T cells; CD16, CD16A, CD25, CD38, CD44, CD56, CD69, CD94, CD335 (NKp46), CD336, (NKp44), CD337 (NKp30), NKp80, NKG2C and NKG2D, DNAM, NCRs for NK cells; CD18, CD64 and CD89 for granulocytes; CD18, CD32, CD64, CD89 and mannose receptor for monocytes and macrophages; CD64 and mannose receptor for dendritic cells; as well as CD35. In certain embodiments, those specificities, i.e. cell surface molecules, of effector cells are suitable for mediating cell killing upon binding of a bispecific or multispecific molecules to such cell surface molecule and, thereby, inducing cytolysis or apoptosis.

As used herein, “multispecific PSMAxCD3 antibody”, “PSMA/CD3 antibody”, “multispecific anti-PSMAxCD3 antibody” or “anti-PSMA/CD3 antibody” refers to a molecule comprising at least one binding domain specifically binding PSMA and at least one binding domain specifically binding CD3. The domains specifically binding PSMA and CD3 are typically VH/VL pairs. The bispecific anti-PSMAxCD3 antibody can be monovalent in terms of its binding to either PSMA or CD3.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule. “Multivalent” refers to the presence of two or more binding sites specific for an antigen in a molecule.

### Antibodies

Provided herein are PSMA antibodies or antigen-binding fragments thereof, nucleic acids and expression vectors encoding the antibodies, recombinant cells containing the vectors, and compositions comprising the antibodies. Methods of making the antibodies, and methods of using the antibodies to treat diseases are also provided. The antibodies disclosed herein possess one or more desirable functional properties, including but not limited to high-affinity binding to PSMA or high specificity to PSMA. In certain embodiments, the antibodies disclosed herein possess the ability to treat or prevent a disease or disorder when administered to a subject alone or in combination with other therapies.

Also provided herein are PSMA bispecific antibodies or antigen-binding fragments thereof, nucleic acids and expression vectors encoding the bispecific antibodies, recombinant cells containing the vectors, and compositions comprising the bispecific antibodies. Methods of making the antibodies, and methods of using the bispecific antibodies to treat diseases, including cancer, are also provided. The antibodies disclosed herein possess one or more desirable functional properties. In some embodiments, the bispecific antibodies provided herein have high-affinity binding to PSMA. In some embodiments, the bispecific antibodies provided herein have high-affinity binding to a second target antigen. In some embodiments, the bispecific antibodies provided herein have high specificity to PSMA. In some embodiments, the bispecific antibodies provided herein have high specificity to a second target antigen. In some, embodiments, the bispecific antibodies provided herein have high specificity to CD3. In some embodiments, the bispecific antibodies provided herein have the ability to treat or prevent a disease or disorder when administered alone. In some embodiments, the bispecific antibodies provided herein have the ability to treat or prevent a disease or disorder when administered in combination with other therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies provided herein can, in certain embodiments, contain a kappa light chain constant domain. The antibodies provided herein can, in certain embodiments, also contain a lambda light chain constant domain. According to particular embodiments, the antibodies provided herein include heavy and/or light chain constant regions from rat or human antibodies. In specific embodiments, the constant region is a human constant region.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH β-sheet framework, or one of three hypervariable regions (LCDR1, LCDR2 or LCDR3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978); Kabat et al., Sequences of Proteins of Immunological Interest, 5th Ed. Public Health Service, National Institutes of Health, Bethesda, MD. (1991)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, MD. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pltickthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions can comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the tables in the Examples below, including Tables 4-12 and 15-20.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region can contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

As used herein, the term an “isolated antibody” refers to an antibody, which is substantially free of other antibodies having different antigenic specificities (e.g., an isolated antibody that specifically binds to PSMA is substantially free of antibodies that do not bind to PSMA). In addition, an isolated antibody is substantially free of other cellular material and/or chemicals. In the case of bispecific PSMAxCD3 antibodies, the bispecific antibody specifically binds both PSMA and CD3, and is substantially free of antibodies that specifically bind antigens other that PSMA and CD3. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

As used herein, the term “monoclonal antibody” refers to an antibody obtained from a population of substantially homogeneous antibodies, i.e., the individual antibodies comprising the population are identical except for possible naturally occurring mutations that can be present in minor amounts. Monoclonal antibodies provided herein can be made by the hybridoma method, phage display technology, single lymphocyte gene cloning technology, or by recombinant DNA methods. For example, the monoclonal antibodies can be produced by a hybridoma, which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide. If the antibody contains a constant region or a portion of the constant region, the constant region also is derived from sequences of human origin.

Human antibody comprises heavy or light chain variable regions that are “derived from” sequences of human origin if the variable regions of the antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci as described herein. “Human antibody” can contain amino acid differences when compared to the human germline immunoglobulin or rearranged immunoglobulin genes due to for example naturally occurring somatic mutations or intentional introduction of substitutions into the framework or antigen binding site, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Human antibodies derived from human immunoglobulin sequences can be generated using systems such as phage display incorporating synthetic CDRs and/or synthetic frameworks, or can be subjected to in vitro mutagenesis to improve antibody properties, resulting in antibodies that are not expressed by the human antibody germline repertoire in vivo. Antibodies in which antigen binding sites are derived from a non-human species are not included in the definition of “human antibody”.

As used herein, the term “humanized antibody” refers to a non-human antibody that is modified to increase the sequence homology to that of a human antibody, such that the antigen-binding properties of the antibody are retained, but its antigenicity in the human body is reduced. Humanized antibody includes an antibody in which the antigen binding sites are derived from non-human species and the variable region frameworks are derived from human immunoglobulin sequences. Humanized antibody can include substitutions in the framework so that the framework may not be an exact copy of expressed human immunoglobulin or human immunoglobulin germline gene sequences.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

“Recombinant” refers to DNA, antibodies and other proteins that are prepared, expressed, created or isolated by recombinant means when segments from different sources are joined to produce recombinant DNA, antibodies or proteins.

“Epitope” refers to a portion of an antigen to which an antibody specifically binds. Epitopes typically consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

“Paratope” refers to a portion of an antibody to which an antigen specifically binds. A paratope can be linear in nature or can be discontinuous, formed by a spatial relationship between non-contiguous amino acids of an antibody rather than a linear series of amino acids. A “light chain paratope” and a “heavy chain paratope” or “light chain paratope amino acid residues” and “heavy chain paratope amino acid residues” refer to antibody light chain and heavy chain residues in contact with an antigen, respectively, or in general, “antibody paratope residues” refer to those antibody amino acids that are in contact with antigen.

“Anti-idiotypic (anti-Id) antibody” is an antibody, which recognizes the antigenic determinants (e.g. the paratope or CDRs) of the antibody. It is generally known in the art the process of producing or preparing an anti-idiotypic antibody. (Lathey, J. et al Immunology 1986 57(1):29-35). The anti-Id antibody can be antigen-blocking or non-blocking. The antigen-blocking anti-Id antibody can be used to detect the free antibody in a sample (e.g. anti-PSMA, anti-CD3 or the bispecific PSMAxCD3 antibody provided herein). The non-blocking anti-Id antibody can be used to detect the total antibody (free, partially bound to antigen, or fully bound to antigen) in a sample. An anti-Id antibody can be prepared by immunizing an animal with the antibody to which an anti-Id antibody is being prepared. In some embodiments described herein, the anti-idiotypic antibody is used for detecting the level of the therapeutic antibodies (e.g. anti-PSMA, anti-CD3 or the bispecific PSMAxCD3 antibody provided herein) in a sample. An anti-Id antibody can also be used as an immunogen to induce an immune response in yet another animal, producing a so-called anti-anti-Id antibody. An anti-anti-Id can be epitopically identical to the original mAb, which induced the anti-Id antibody. Thus, by using antibodies to the idiotypic determinants of a mAb, it is possible to identify other clones expressing antibodies of identical specificity. Anti-Id antibodies can be varied (thereby producing anti-Id antibody variants) and/or derivatized by any suitable technique, such as those described elsewhere herein with respect to the antibodies specifically binding PSMA or CD3, or the bispecific PSMAxCD3 antibodies.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a PSMA antigen) and a second immunoglobulin variable domain sequence that has binding specificity for a second epitope. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on PSMA and the second epitope is located on CD3.

As used herein, the term “prostate-specific membrane antigen” or “PSMA” refers to a type II membrane protein expressed on certain cells. The amino acid sequence of the Pan troglodytes (also referred to as chimpanzee or chimp) PSMA is shown in SEQ ID NO:1416 (H2Q3K5_PANTR). The extracellular domain spans residues 44-750, the transmembrane domain spans residues 20-43 and the cytoplasmic domain spans residues 1-19 of SEQ ID NO:1416.

(SEQ ID NO: 1416)
MWNLLHETDSAVATARRPRWLCAGALVLAGGFFLLGFLFG
WFIKSSNEATNITPKHNMKAFLDELKAENIKKFLYNFTQI
PHLAGTEQNFQLAKQIQSQWKEFGLDSVELAHYDVLLSYP
NKTHPNYISIINEDGNEIFNTSLFEPPPPGYENVLDIVPP
FSAFSPQGMPEGDLVYVNYARTEDFFKLERDMKINCSGKI
VIARYGKVFRGNKVKNAQLAGAKGVILYSDPADYFAPGVK
SYPDGWNLPGGGVQRGNILNLNGAGDPLTPGYPANEYAYR
HGIAEAVGLPSIPVHPIGYYDAQKLLEKMGGSAPPDSSWR
GSLKVPYNVGPGFTGNFSTQKVKMHIHSTNEVTRIYNVIG
TLRGAVEPDRYVILGGHRDSWVFGGIDPQSGAAVVHEIVR
SFGTLKKEGWRPRRTILFASWDAEEFGLLGSTEWAEENSR
LLQERGVAYINADSSIEGNYTLRVDCTPLMYSLVYNLTKE
LKSPDEGFEGKSLYESWTKKSPSPEFSGMPRISKLGSGND
FEVFFQRLGIASGRARYTKNWETNKFSGYPLYHSVYETYE
LVEKFYDPMFKYHLTVAQVRGGMVFELANSIVLPFDCRDY
AVVLRKYADKIYNISMKHPQEMKTYSVSFDSLFSAVKNFT
EIASKFTERLQDEDKSNPILLRMMNDQLMFLERAFIDPLG
LPDRPFYRHVIYAPSSHNKYAGESFPGIYDALFDIESKVD
PSKAWGDVKRQISVAAFTVQAAAETLSEVA

The amino acid sequence of the Macaca fascicularis (also referred to as cynomolgus monkey, macaque or cyno) PSMA is shown in SEQ ID NO:1417 (EHH56646.1). The extracellular domain spans residues 44-750, the transmembrane domain spans residues 20-43 and the cytoplasmic domain spans residues 1-19 of SEQ ID NO:1417.

(SEQ ID NO: 1417)
MWNLLHETDSAVATARRPRWLCAGALVLAGGFFLLGELFG
WFIKSSSEATNITPKHNMKAFLDELKAENIKKFLHNFTQI
PHLAGTEQNFQLAKQIQSQWKEFGLDSVELTHYDVLLSYP
NKTHPNYISIINEDGNEIFNTSLFEPPPAGYENVSDIVPP
FSAFSPQGMPEGDLVYVNYARTEDFFKLERDMKINCSGKI
VIARYGKVFRGNKVKNAQLAGATGVILYSDPDDYFAPGVK
SYPDGWNLPGGGVQRGNILNLNGAGDPLTPGYPANEYAYR
RGMAEAVGLPSIPVHPIGYYDAQKLLEKMGGSASPDSSWR
GSLKVPYNVGPGFTGNFSTQKVKMHIHSTSEVTRIYNVIG
TLRGAVEPDRYVILGGHRDSWVFGGIDPQSGAAVVHEIVR
SFGMLKKEGWRPRRTILFASWDAEEFGLLGSTEWAEENSR
LLQERGVAYINADSSIEGNYTLRVDCTPLMYSLVYNLTKE
LESPDEGFEGKSLYESWTKKSPSPEFSGMPRISKLGSGND
FEVFFQRLGIASGRARYTKNWETNKFSSYPLYHSVYETYE
LVEKFYDPMFKYHLTVAQVRGGMVFELANSVVLPFDCRDY
AVVLRKYADKIYNISMKHPQEMKTYSVSFDSLFSAVKNFT
EIASKFSERLRDFDKSNPILLRMMNDQLMFLERAFIDPLG
LPDRPFYRHVIYAPSSHNKYAGESFPGIYDALFDIESKVD
PSQAWGEVKRQISIATFTVQAAAETLSEVA

The amino acid sequence of the human PSMA is shown in SEQ ID NO:1418. The extracellular domain spans residues 44-750, the transmembrane domain spans residues 20-43 and the cytoplasmic domain spans residues 1-19 of SEQ ID NO:1418.

(SEQ ID NO: 1418)
MWNLLHETDSAVATARRPRWLCAGALVLAGGFFLLGFLFG
WFIKSSNEATNITPKHNMKAFLDELKAENIKKFLYNFTQI
PHLAGTEQNFQLAKQIQSQWKEFGLDSVELAHYDVLLSYP
NKTHPNYISIINEDGNEIFNTSLFEPPPPGYENVSDIVPP
FSAFSPQGMPEGDLVYVNYARTEDFFKLERDMKINCSGKI
VIARYGKVFRGNKVKNAQLAGAKGVILYSDPADYFAPGVK
SYPDGWNLPGGGVQRGNILNLNGAGDPLTPGYPANEYAYR
RGIAEAVGLPSIPVHPIGYYDAQKLLEKMGGSAPPDSSWR
GSLKVPYNVGPGFTGNFSTQKVKMHIHSTNEVTRIYNVIG
TLRGAVEPDRYVILGGHRDSWVFGGIDPQSGAAVVHEIVR
SFGTLKKEGWRPRRTILFASWDAEEFGLLGSTEWAEENSR
LLQERGVAYINADSSIEGNYTLRVDCTPLMYSLVHNLTKE
LKSPDEGFEGKSLYESWTKKSPSPEFSGMPRISKLGSGND
FEVFFQRLGIASGRARYTKNWETNKFSGYPLYHSVYETYE
LVEKFYDPMFKYHLTVAQVRGGMVFELANSIVLPFDCRDY
AVVLRKYADKIYSISMKHPQEMKTYSVSFDSLFSAVKNFT
EIASKFSERLQDEDKSNPIVLRMMNDQLMFLERAFIDPLG
LPDRPFYRHVIYAPSSHNKYAGESFPGIYDALFDIESKVD
PSKAWGEVKRQIYVAAFTVQAAAETLSEVA

The term “PSMA” includes any PSMA variant, isoform, and species homolog, which is naturally expressed by cells (including prostate cells) or can be expressed on cells transfected with genes or cDNA encoding the polypeptide. In specific embodiments, the PSMA is a human PSMA.

As used herein, the term “CD3” refers to an antigen that is expressed on T cells as part of the multimeric T cell receptor (TCR) complex and which consists of a homodimer or heterodimer formed from the association of two or four receptor chains: CD3 epsilon, CD3 delta, CD3 zeta and CD3 gamma. In some embodiments, CD3 antibodies provided herein bind to the CD3-epsilon polypeptide, which together with CD3-gamma, -delta and -zeta, and the T cell receptor alpha/beta and gamma/delta heterodimers, forms the T cell receptor-CD3 complex. This complex plays an important role in coupling antigen recognition to several intracellular signal-transduction pathways. The CD3 complex mediates signal transduction, resulting in T cell activation and proliferation. CD3 is required for the immune response. The term “CD3” includes any CD3 variant, isoform, and species homolog, which is naturally expressed by cells (including T cells) or can be expressed on cells transfected with genes or cDNA encoding the polypeptide. In specific embodiments, the CD3 is a human CD3. All references to proteins, polypeptides and protein fragments herein are intended to refer to the human version of the respective protein, polypeptide or protein fragment unless explicitly specified as being from a non-human species. Thus, “CD3” means human CD3 unless specified as being from a non-human species, e.g., “mouse CD3” “monkey CD3,” etc.

An exemplary human CD3 epsilon comprises the amino acid sequence of SEQ ID NO:1021.

(SEQ ID NO: 1021)
MQSGTHWRVLGLCLLSVGVWGQDGNEEMGGITQTPYKVSI
SGTTVILTCPQYPGSEILWQHNDKNIGGDEDDKNIGSDED
HLSLKEFSELEQSGYYVCYPRGSKPEDANFYLYLRARVCE
NCMEMDVMSVATIVIVDICITGGLLLLVYYWSKNRKAKAK
PVTRGAGAGGRQRGQNKERPPPVPNPDYEPIRKGQRDLYS
GLNQRRI

An exemplary extracellular domain of a human CD3 epsilon comprises the amino acid sequence of SEQ ID NO:1022.

(SEQ ID NO: 1022)
DGNEEMGGITQTPYKVSISGTTVILTCPQYPGSEILWQHN
DKNIGGDEDDKNIGSDEDHLSLKEFSELEQSGYYVCYPRG
SKPEDANFYLYLRARVCENCMEMD

“Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (KD) of about 1×10⁻⁷ M or less, about 5×10⁻⁸ M or less, about 1×10⁻⁸ M or less, or about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein). The dissociation constant can be measured using standard procedures. Antibodies that specifically bind to the antigen or the epitope within the antigen can, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody specifically binds one antigen or one epitope, a bispecific antibody specifically binds two distinct antigens or two distinct epitopes.

Throughout the specification, “CD3-specific” or “specifically binds CD3” or “anti-CD3 antibody” refers to antibodies that bind specifically to the CD3-epsilon polypeptide (SEQ ID NO:1021), including antibodies that bind specifically to the CD3-epsilon extracellular domain (ECD) (SEQ ID NO:1022). CD3-epsilon, together with CD3-gamma, -delta and -zeta, and the T cell receptor alpha/beta and gamma/delta heterodimers, forms the T cell receptor-CD3 complex. This complex plays an important role in coupling antigen recognition to several intracellular signal-transduction pathways. The CD3 complex mediates signal transduction, resulting in T cell activation and proliferation. CD3 is required for the immune response.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

As used herein, “Tagg” refers to the temperature at which the protein starts to aggregate either through dimerization or oligomerization. The aggregation temperature detects the onset of aggregation, the temperature at which a protein will show a tendency to aggregate. Tagg can be determined by differential scanning calorimetry (DSC), Differential Scanning Fluorimetry (DSF) or by circular dichroism (CD). These techniques can detect small changes in the conformation of the protein and therefore detect the starting point of aggregation. Tagg values can be lower or higher than Tm. In cases where Tagg is lower than Tm, the protein either dimerizes and/or oligomerizes first and then starts unfolding later at higher temperatures than the Tagg. In cases where Tagg is higher than Tm, the protein starts to unfold first and then aggregates at a higher temperature than the Tm. Both events are commonly observed and depend on amino acid composition and protein conformation.

As used herein, “Tm” or “mid-point temperature” “is the temperature midpoint of a thermal unfolding curve. It refers to the temperature where 50% of the amino acid sequence is in its native conformation and the other 50% is denatured. A thermal unfolding curve is typically plotted as a function of temperature. Tm is used to measure protein stability. In general, a higher Tm is an indication of a more stable protein. The Tm can be readily determined using methods well known to those skilled in the art such as Circular Dichroism Spectroscopy, Differential Scanning calorimetry, Differential Scanning Fluorimetry (both intrinsic and extrinsic dye based), UV spectroscopy, FT-IR and Isothermal calorimetry (ITC).

In one aspect, provided herein is an antibody that binds to PSMA. In some embodiments, the antibody comprises a heavy chain variable region and a light chain variable region. In some embodiments, the PSMA antibody is a humanized antibody. In some embodiments, the PSMA antibody is a human antibody.

In certain embodiments, provided herein is a PSMA antibody comprising a VH region, VL region, VH CDR1, VH CDR2, VH CDR3, VL CDR1, VL CDR2, and/or VL CDR3 of any one of the antibodies described herein. In some embodiments, provided herein is a PSMA antibody comprising a VH region of any one of the antibodies described herein. In some embodiments, provided herein is a PSMA antibody comprising a VL region of any one of the antibodies described herein. In some embodiments, provided herein is a PSMA antibody comprising a VH region of any one of the antibodies described herein, and a VL region of any one of the antibodies described herein. In some embodiments, provided herein is a PSMA antibody comprising a VH CDR1, VH CDR2, and VH CDR3 of any one of the antibodies described herein. In some embodiments, provided herein is a PSMA antibody comprising a VL CDR1, VL CDR2, and VL CDR3 of any one of the antibodies described herein. In some embodiments, provided herein is a PSMA antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of PSMA antibodies provided herein are provided in Tables 4-12.

In certain embodiments, provided herein is a PSMA bispecific antibody comprising a binding domain that binds to PSMA having a VH region, VL region, VH CDR1, VH CDR2, VH CDR3, VL CDR1, VL CDR2, and/or VL CDR3 of any one of the antibodies described herein. In some embodiments, provided herein is a PSMA bispecific antibody comprising a binding domain that binds to PSMA having a VH region of any one of the antibodies described herein. In some embodiments, provided herein is a PSMA bispecific antibody comprising a binding domain that binds to PSMA having a VL region of any one of the antibodies described herein. In some embodiments, provided herein is a PSMA bispecific antibody comprising a binding domain that binds to PSMA having a VH region of any one of the antibodies described herein, and a VL region of any one of the antibodies described herein. In some embodiments, provided herein is a PSMA bispecific antibody comprising a binding domain that binds to PSMA having a VH CDR1, VH CDR2, and VH CDR3 of any one of the antibodies described. In some embodiments, provided herein is a PSMA bispecific antibody comprising a binding domain that binds to PSMA having a VL CDR1, VL CDR2, and VL CDR3 of any one of the antibodies described herein. In some embodiments, provided herein is a PSMA bispecific antibody comprising a binding domain that binds to PSMA having a VH CDR1, VH CDR2, and VH CDR3 of any one of the antibodies described herein; and a VL CDR1, VL CDR2, and VL CDR3 of any one of the antibodies described herein. In certain embodiments, the PSMA antibody is a bispecific antibody. In some embodiments, the PSMA bispecific antibody further comprises a second binding domain that binds to CD3 having a VH region, VL region, VH CDR1, VH CDR2, VH CDR3, VL CDR1, VL CDR2, and/or VL CDR3 of a CD3 antibody provided herein. In some embodiments, the PSMA bispecific antibody further comprises a second binding domain that binds to CD3 having a VH region of a CD3 antibody provided herein. In some embodiments, the PSMA bispecific antibody further comprises a second binding domain that binds to CD3 having a VL region of a CD3 antibody provided herein. In some embodiments, the PSMA bispecific antibody further comprises a second binding domain that binds to CD3 having a VH region of a CD3 antibody provided herein, and a VL region of a CD3 antibody provided herein. In some embodiments, the PSMA bispecific antibody further comprises a second binding domain that binds to CD3 having a VH CDR1, VH CDR2, and VH CDR3 of a CD3 antibody provided herein. In some embodiments, the PSMA bispecific antibody further comprises a second binding domain that binds to CD3 having a VL CDR1, VL CDR2, and VL CDR3 of a CD3 antibody provided herein. In some embodiments, the PSMA bispecific antibody further comprises a second binding domain that binds to CD3 having a VH CDR1, VH CDR2, and VH CDR3 of a CD3 antibody provided herein, and a VL CDR1, VL CDR2, and VL CDR3 of a CD3 antibody provided herein.

In some embodiments, the antibody specifically binds PSMA. In some embodiments, the antibody specifically binds to Pan troglodytes (chimpanzee, chimp) PSMA. In other embodiments, the antibody specifically binds to Macaca fascicularis (cynomolgus monkey, macaque, cyno) PSMA. In yet other embodiments, the antibody specifically binds to and/or human PSMA. In some embodiments, the antibody specifically binds to Pan troglodytes, Macaca fascicularis, and human PSMA. In specific embodiments, the antibody specifically binds to both cyno and human PSMA.

In certain embodiments, the PSMA antibodies bind to the chimpanzee target antigen. In one embodiment, the antibodies bind to the human and macaque PSMA target antigens with affinities within 5-fold of each other. In other words, the difference in antibody binding is less than a multiple of 5. In this case, the identical antibody molecule can be used both for preclinical evaluation of safety, activity and/or pharmacokinetic profile of PSMA in primates and as a drug in humans. Put in other words, the same PSMA-specific molecule can be used in preclinical animal studies as well as in clinical studies in humans. This leads to highly comparable results and a much-increased predictive power of the animal studies compared to species-specific surrogate molecules. Since the PSMA domain is cross-species specific, i.e. reactive with the human and macaque antigens, the antibody or fragments thereof can be used both for preclinical evaluation of safety, activity and/or pharmacokinetic profile of these binding domains in primates and—in the identical form—as drug in humans.

In other embodiments, the PSMA is present on the surface of a cell.

In some embodiments, the antibody is a humanized antibody. In some embodiments, the antibody is a human antibody.

In certain embodiments, the antibody is an IgG antibody. In other embodiments, the IgG antibody is an IgG1, IgG2, IgG3, or IgG4 antibody.

In some embodiments, a multispecific PSMAxCD3 antibody provided herein is an IgG1, an IgG2, an IgG3 or an IgG4 isotype. In some embodiments, a multispecific PSMAxCD3 antibody provided herein is an IgG1 isotype. In some embodiments, a multispecific PSMAxCD3 antibody provided herein is an IgG2 isotype. In some embodiments, a multispecific PSMAxCD3 antibody provided herein is an IgG3 isotype. In some embodiments, a multispecific PSMAxCD3 antibody provided herein is an IgG4 isotype.

Immunogenicity of therapeutic antibodies can associated with increased risk of infusion reactions and decreased duration of therapeutic response (Baert et al., (2003) N Engl J Med 348:602-08). The extent to which therapeutic antibodies induce an immune response in the host can be determined in part by the allotype of the antibody (Stickler et al., (2011) Genes and Immunity 12:213-21). Antibody allotype is related to amino acid sequence variations at specific locations in the constant region sequences of the antibody. The table below shows select IgG1, IgG2 and IgG4 allotypes of some embodiments.

| | Amino acid residue at position of diversity (residue | | | | | | | |
|---|---|---|---|---|---|---|---|---|
| | numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n-) | P | V | | | | | | |
| G2m(n)(n-) | T | V | | | | | | |
| GnG4m(a) | | | L | R | | | | |
| F1m(17) | | | | | K | E | M | A |
| G1m(17, 1) | | | | | K | D | L | A |

In some embodiments, a PSMA antibody provided herein is an G2m(n) allotype. In some embodiments, a PSMA antibody provided herein is an G2m(n−) allotype. In some embodiments, a PSMA antibody provided herein is an G2m(n)/(n−) allotype. In some embodiments, a PSMA antibody provided herein is an nG4m(a) allotype. In some embodiments, a PSMA antibody provided herein is an G1m(17) allotype. In some embodiments, a PSMA antibody provided herein is an G1m(17,1) allotype.

In some embodiments, a multispecific PSMAxCD3 antibody provided herein is an G2m(n) allotype. In some embodiments, a multispecific PSMAxCD3 antibody provided herein is an G2m(n−) allotype. In some embodiments, a multispecific PSMAxCD3 antibody provided herein is an G2m(n)/(n−) allotype. In some embodiments, a multispecific PSMAxCD3 antibody provided herein is an nG4m(a) allotype. In some embodiments, a multispecific PSMAxCD3 antibody provided herein is an G1m(17) allotype. In some embodiments, a multispecific PSMAxCD3 antibody provided herein is an G1m(17,1) allotype.

In some embodiments, the antibody is a bispecific antibody. In certain embodiments, the antibody is multivalent. In other embodiments, the antibody is capable of binding at least three antigens. In some embodiments, the antibody is capable of binding at least five antigens.

In certain embodiments, provided is a PSMA antibody that is an intact antibody. In other embodiments, provided is a PSMA antibody is an antigen binding fragment of the PSMA antibody. In some embodiments, the antigen binding fragment of the PSMA antibody is a functional fragment.

In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the PSMA antibody comprises a VH region and a VL region. In some embodiments, the PSMA antibody is a single chain antibody. In some embodiments, the PSMA antibody is a single domain antibody. In some embodiments, the PSMA antibody is a nanobody. In certain embodiments, the PSMA antibody is a VHH antibody. In certain embodiments, the PSMA antibody is a llama antibody. In some embodiments, the PSMA antibody is a multispecific antibody. In other embodiments, the PSMA is a bispecific antibody. In certain embodiments, the multispecific antibody comprises an antigen binding fragment of a PSMA antibody provided herein. In other embodiments, the bispecific antibody comprises an antigen binding fragment of a PSMA antibody provided herein. In some embodiments, the PSMA antibody is an agonistic antibody. In other embodiments, the PSMA antibody is an antagonistic antibody.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to a combination of the numbering systems provided herein. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein, including in the Examples and Tables 4-12 (PSMA antibodies), Tables 16-22 (CD3 antibodies), and Tables 23-28 (PSMAxCD3 antibodies) herein. Other sets of CDRs are contemplated and within the scope of the antibody embodiments provided herein.

In one aspect, provided herein is an antibody that binds PSMA, comprising: (i) a VH comprising a VH CDR1, a VH CDR2, and a VH CDR3 having an amino acid sequence of SEQ ID NOs:1, 2, and 3, respectively, and (ii) a VL comprising a VL CDR1, VL CDR2, and VL CDR3 having an amino acid sequence of SEQ ID NOs:16, 5, and 6, respectively. In one aspect, provided herein is an antibody that binds PSMA, comprising: (i) a VH comprising a VH CDR1, a VH CDR2, and a VH CDR3 having an amino acid sequence of SEQ ID NOs:7, 4, and 9, respectively, and (ii) a VL comprising a VL CDR1, VL CDR2, and VL CDR3 having an amino acid sequence of SEQ ID NOs:10, 11, and 12, respectively. In one aspect, provided herein is an antibody that binds PSMA, comprising: (i) a VH comprising a VH CDR1, a VH CDR2, and a VH CDR3 having an amino acid sequence of SEQ ID NOs:13, 14, and 3, respectively, and (ii) a VL comprising a VL CDR1, VL CDR2, and VL CDR3 having an amino acid sequence of SEQ ID NOs:16, 5, and 6, respectively. In one aspect, provided herein is an antibody that binds PSMA, comprising: (i) a VH comprising a VH CDR1, a VH CDR2, and a VH CDR3 having an amino acid sequence of SEQ ID NOs:19, 20, and 21, respectively, and (ii) a VL comprising a VL CDR1, VL CDR2, and VL CDR3 having an amino acid sequence of SEQ ID NOs:22, 23, and 24, respectively. In one aspect, provided herein is an antibody that binds PSMA, comprising: (i) a VH comprising a VH CDR1, a VH CDR2, and a VH CDR3 having an amino acid sequence of SEQ ID NOs:25, 26, and 27, respectively, and (ii) a VL comprising a VL CDR1, VL CDR2, and VL CDR3 having an amino acid sequence of SEQ ID NOs:28, 11, and 6, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:31; and (ii) a VL comprising a VL CDR1, a VL CDR2, and a VL CDR3 having an amino acid sequence of a VL CDR1, a VL CDR2, and a VL CDR3, respectively, of SEQ ID NO:32. In one aspect, provided herein is an antibody that binds PSMA, comprising a VH having an amino acid sequence of SEQ ID NO:31. In one aspect, provided herein is an antibody that binds PSMA, comprising a VL having an amino acid sequence of SEQ ID NO:32. In one aspect, provided herein is an antibody that binds PSMA, comprising a VH having an amino acid sequence of SEQ ID NO:31, and a VL having an amino acid sequence of SEQ ID NO:32. In one aspect, provided herein is an antibody that binds PSMA, comprising a heavy chain having an amino acid sequence of SEQ ID NO:33. In one aspect, provided herein is an antibody that binds PSMA, comprising a light chain having an amino acid sequence of SEQ ID NO:34. In one aspect, provided herein is an antibody that binds PSMA, comprising a heavy chain having an amino acid sequence of SEQ ID NO:33, and a light chain having an amino acid sequence of SEQ ID NO:34. In one aspect, provided herein is an antibody that binds PSMA, comprising a VH having an amino acid sequence having at least 95% identity to an amino acid sequence of SEQ ID NO:31. In one aspect, provided herein is an antibody that binds PSMA, comprising a VL having an amino acid sequence having at least 95% identity to an amino acid sequence of SEQ ID NO:32. In one aspect, provided herein is an antibody that binds PSMA, comprising a VH having an amino acid sequence having at least 95% identity to an amino acid sequence of SEQ ID NO:31, and a VL having an amino acid sequence having at least 95% identity to an amino acid sequence of SEQ ID NO:32. In one aspect, provided herein is an antibody that binds PSMA, comprising a heavy chain having an amino acid sequence having at least 95% identity to an amino acid sequence of SEQ ID NO:33. In one aspect, provided herein is an antibody that binds PSMA, comprising a light chain having an amino acid sequence having at least 95% identity to an amino acid sequence of SEQ ID NO:34. In one aspect, provided herein is an antibody that binds PSMA, comprising a heavy chain having an amino acid sequence having at least 95% identity to an amino acid sequence of SEQ ID NO:33, and a light chain having an amino acid sequence having at least 95% identity to an amino acid sequence of SEQ ID NO:34.

In one aspect, provided herein is an antibody that binds PSMA, comprising: (i) a VH comprising a VH CDR1, a VH CDR2, and a VH CDR3 having an amino acid sequence of SEQ ID NOs:1, 2, and 3, respectively, and (ii) a VL comprising a VL CDR1, VL CDR2, and VL CDR3 having an amino acid sequence of SEQ ID NOs:38, 39, and 40, respectively. In one aspect, provided herein is an antibody that binds PSMA, comprising: (i) a VH comprising a VH CDR1, a VH CDR2, and a VH CDR3 having an amino acid sequence of SEQ ID NOs:7, 42, and 9, respectively, and (ii) a VL comprising a VL CDR1, VL CDR2, and VL CDR3 having an amino acid sequence of SEQ ID NOs:44, 45, and 46, respectively. In one aspect, provided herein is an antibody that binds PSMA, comprising: (i) a VH comprising a VH CDR1, a VH CDR2, and a VH CDR3 having an amino acid sequence of SEQ ID NOs:13, 14, and 3, respectively, and (ii) a VL comprising a VL CDR1, VL CDR2, and VL CDR3 having an amino acid sequence of SEQ ID NOs:38, 39, and 40, respectively. In one aspect, provided herein is an antibody that binds PSMA, comprising: (i) a VH comprising a VH CDR1, a VH CDR2, and a VH CDR3 having an amino acid sequence of SEQ ID NOs:19, 20, and 21, respectively, and (ii) a VL comprising a VL CDR1, VL CDR2, and VL CDR3 having an amino acid sequence of SEQ ID NOs:56, 57, and 58, respectively. In one aspect, provided herein is an antibody that binds PSMA, comprising: (i) a VH comprising a VH CDR1, a VH CDR2, and a VH CDR3 having an amino acid sequence of SEQ ID NOs:25, 26, and 27, respectively, and (ii) a VL comprising a VL CDR1, VL CDR2, and VL CDR3 having an amino acid sequence of SEQ ID NOs:62, 45, and 40, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:31; and (ii) a VL comprising a VL CDR1, a VL CDR2, and a VL CDR3 having an amino acid sequence of a VL CDR1, a VL CDR2, and a VL CDR3, respectively, of SEQ ID NO:66. In one aspect, provided herein is an antibody that binds PSMA, comprising a VH having an amino acid sequence of SEQ ID NO:31. In one aspect, provided herein is an antibody that binds PSMA, comprising a VL having an amino acid sequence of SEQ ID NO:66. In one aspect, provided herein is an antibody that binds PSMA, comprising a VH having an amino acid sequence of SEQ ID NO:31, and a VL having an amino acid sequence of SEQ ID NO:66. In one aspect, provided herein is an antibody that binds PSMA, comprising a heavy chain having an amino acid sequence of SEQ ID NO:33. In one aspect, provided herein is an antibody that binds PSMA, comprising a light chain having an amino acid sequence of SEQ ID NO:68. In one aspect, provided herein is an antibody that binds PSMA, comprising a heavy chain having an amino acid sequence of SEQ ID NO:33, and a light chain having an amino acid sequence of SEQ ID NO:68. In one aspect, provided herein is an antibody that binds PSMA, comprising a VH having an amino acid sequence having at least 95% identity to an amino acid sequence of SEQ ID NO:31. In one aspect, provided herein is an antibody that binds PSMA, comprising a VL having an amino acid sequence having at least 95% identity to an amino acid sequence of SEQ ID NO:66. In one aspect, provided herein is an antibody that binds PSMA, comprising a VH having an amino acid sequence having at least 95% identity to an amino acid sequence of SEQ ID NO:31, and a VL having an amino acid sequence having at least 95% identity to an amino acid sequence of SEQ ID NO:66. In one aspect, provided herein is an antibody that binds PSMA, comprising a heavy chain having an amino acid sequence having at least 95% identity to an amino acid sequence of SEQ ID NO:33. In one aspect, provided herein is an antibody that binds PSMA, comprising a light chain having an amino acid sequence having at least 95% identity to an amino acid sequence of SEQ ID NO:68. In one aspect, provided herein is an antibody that binds PSMA, comprising a heavy chain having an amino acid sequence having at least 95% identity to an amino acid sequence of SEQ ID NO:33, and a light chain having an amino acid sequence having at least 95% identity to an amino acid sequence of SEQ ID NO:68.

In one aspect, provided herein is an antibody that binds PSMA, comprising: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds PSMA, comprising: (i) a VH comprising a VH CDR1, a VH CDR2, and a VH CDR3 having an amino acid sequence of SEQ ID NOs:75, 76, and 77, respectively, and (ii) a VL comprising a VL CDR1, VL CDR2, and VL CDR3 having an amino acid sequence of SEQ ID NOs:78, 79, and 80, respectively. In one aspect, provided herein is an antibody that binds PSMA, comprising: (i) a VH comprising a VH CDR1, a VH CDR2, and a VH CDR3 having an amino acid sequence of SEQ ID NOs:81, 82, and 71, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds PSMA, comprising: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:90, 91, and 92, respectively. In one aspect, provided herein is an antibody that binds PSMA, comprising: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:96, 79, and 74, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In one aspect, provided herein is an antibody that binds PSMA, comprising a VH having an amino acid sequence of SEQ ID NO:99. In one aspect, provided herein is an antibody that binds PSMA, comprising a VL having an amino acid sequence of SEQ ID NO:100. In one aspect, provided herein is an antibody that binds PSMA, comprising a VH having an amino acid sequence of SEQ ID NO:99, and a VL having an amino acid sequence of SEQ ID NO:100. In one aspect, provided herein is an antibody that binds PSMA, comprising a heavy chain having an amino acid sequence of SEQ ID NO:101. In one aspect, provided herein is an antibody that binds PSMA, comprising a light chain having an amino acid sequence of SEQ ID NO:102. In one aspect, provided herein is an antibody that binds PSMA, comprising a heavy chain having an amino acid sequence of SEQ ID NO:101, and a light chain having an amino acid sequence of SEQ ID NO:102. In one aspect, provided herein is an antibody that binds PSMA, comprising a VH having an amino acid sequence having at least 95% identity to an amino acid sequence of SEQ ID NO:99. In one aspect, provided herein is an antibody that binds PSMA, comprising a VL having an amino acid sequence having at least 95% identity to an amino acid sequence of SEQ ID NO:100. In one aspect, provided herein is an antibody that binds PSMA, comprising a VH having an amino acid sequence having at least 95% identity to an amino acid sequence of SEQ ID NO:99, and a VL having an amino acid sequence having at least 95% identity to an amino acid sequence of SEQ ID NO:100. In one aspect, provided herein is an antibody that binds PSMA, comprising a heavy chain having an amino acid sequence having at least 95% identity to an amino acid sequence of SEQ ID NO:101. In one aspect, provided herein is an antibody that binds PSMA, comprising a light chain having an amino acid sequence having at least 95% identity to an amino acid sequence of SEQ ID NO:102. In one aspect, provided herein is an antibody that binds PSMA, comprising a heavy chain having an amino acid sequence having at least 95% identity to an amino acid sequence of SEQ ID NO:101, and a light chain having an amino acid sequence having at least 95% identity to an amino acid sequence of SEQ ID NO:102.

In one aspect, provided herein is an antibody that binds PSMA, comprising: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:106, 107, and 108, respectively. In one aspect, provided herein is an antibody that binds PSMA, comprising: (i) a VH comprising a VH CDR1, a VH CDR2, and a VH CDR3 having an amino acid sequence of SEQ ID NOs:75, 76, and 77, respectively, and (ii) a VL comprising a VL CDR1, VL CDR2, and VL CDR3 having an amino acid sequence of SEQ ID NOs:112, 113, and 114, respectively. In one aspect, provided herein is an antibody that binds PSMA, comprising: (i) a VH comprising a VH CDR1, a VH CDR2, and a VH CDR3 having an amino acid sequence of SEQ ID NOs:81, 82, and 71, respectively, and (ii) a VL comprising a VL CDR1, VL CDR2, and VL CDR3 having an amino acid sequence of SEQ ID NOs:106, 107, and 108, respectively. In one aspect, provided herein is an antibody that binds PSMA, comprising: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:124, 125, and 126, respectively. In one aspect, provided herein is an antibody that binds PSMA, comprising: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:130, 113, and 108, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:134. In one aspect, provided herein is an antibody that binds PSMA, comprising a VH having an amino acid sequence of SEQ ID NO:99. In one aspect, provided herein is an antibody that binds PSMA, comprising a VL having an amino acid sequence of SEQ ID NO:134. In one aspect, provided herein is an antibody that binds PSMA, comprising a VH having an amino acid sequence of SEQ ID NO:99, and a VL having an amino acid sequence of SEQ ID NO:134. In one aspect, provided herein is an antibody that binds PSMA, comprising a heavy chain having an amino acid sequence of SEQ ID NO:101. In one aspect, provided herein is an antibody that binds PSMA, comprising a light chain having an amino acid sequence of SEQ ID NO:136. In one aspect, provided herein is an antibody that binds PSMA, comprising a heavy chain having an amino acid sequence of SEQ ID NO:101, and a light chain having an amino acid sequence of SEQ ID NO:136. In one aspect, provided herein is an antibody that binds PSMA, comprising a VH having an amino acid sequence having at least 95% identity to an amino acid sequence of SEQ ID NO:99. In one aspect, provided herein is an antibody that binds PSMA, comprising a VL having an amino acid sequence having at least 95% identity to an amino acid sequence of SEQ ID NO:134. In one aspect, provided herein is an antibody that binds PSMA, comprising a VH having an amino acid sequence having at least 95% identity to an amino acid sequence of SEQ ID NO:99, and a VL having an amino acid sequence having at least 95% identity to an amino acid sequence of SEQ ID NO:134. In one aspect, provided herein is an antibody that binds PSMA, comprising a heavy chain having an amino acid sequence having at least 95% identity to an amino acid sequence of SEQ ID NO:101. In one aspect, provided herein is an antibody that binds PSMA, comprising a light chain having an amino acid sequence having at least 95% identity to an amino acid sequence of SEQ ID NO:136. In one aspect, provided herein is an antibody that binds PSMA, comprising a heavy chain having an amino acid sequence having at least 95% identity to an amino acid sequence of SEQ ID NO:101, and a light chain having an amino acid sequence having at least 95% identity to an amino acid sequence of SEQ ID NO:136.

In one aspect, provided herein is an antibody that binds PSMA, comprising: (i) a VH comprising a VH CDR1, a VH CDR2, and a VH CDR3 having an amino acid sequence of SEQ ID NOs:137, 138, and 139, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds PSMA, comprising: (i) a VH comprising a VH CDR1, a VH CDR2, and a VH CDR3 having an amino acid sequence of SEQ ID NOs:143, 144, and 145, respectively, and (ii) a VL comprising a VL CDR1, VL CDR2, and VL CDR3 having an amino acid sequence of SEQ ID NOs:78, 79, and 148, respectively. In one aspect, provided herein is an antibody that binds PSMA, comprising: (i) a VH comprising a VH CDR1, a VH CDR2, and a VH CDR3 having an amino acid sequence of SEQ ID NOs:149, 150, and 139, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds PSMA, comprising: (i) a VH comprising a VH CDR1, a VH CDR2, and a VH CDR3 having an amino acid sequence of SEQ ID NOs:155, 156, and 157, respectively, and (ii) a VL comprising a VL CDR1, VL CDR2, and VL CDR3 having an amino acid sequence of SEQ ID NOs:90, 91, and 92, respectively. In one aspect, provided herein is an antibody that binds PSMA, comprising: (i) a VH comprising a VH CDR1, a VH CDR2, and a VH CDR3 having an amino acid sequence of SEQ ID NOs:161, 162, and 163, respectively, and (ii) a VL comprising a VL CDR1, VL CDR2, and VL CDR3 having an amino acid sequence of SEQ ID NOs:79, 165, and 74, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:167; and (ii) a VL comprising a VL CDR1, a VL CDR2, and a VL CDR3 having an amino acid sequence of a VL CDR1, a VL CDR2, and a VL CDR3, respectively, of SEQ ID NO:100. In one aspect, provided herein is an antibody that binds PSMA, comprising a VH having an amino acid sequence of SEQ ID NO:167. In one aspect, provided herein is an antibody that binds PSMA, comprising a VL having an amino acid sequence of SEQ ID NO:100. In one aspect, provided herein is an antibody that binds PSMA, comprising a VH having an amino acid sequence of SEQ ID NO:167, and a VL having an amino acid sequence of SEQ ID NO:100. In one aspect, provided herein is an antibody that binds PSMA, comprising a heavy chain having an amino acid sequence of SEQ ID NO:169. In one aspect, provided herein is an antibody that binds PSMA, comprising a light chain having an amino acid sequence of SEQ ID NO:102. In one aspect, provided herein is an antibody that binds PSMA, comprising a heavy chain having an amino acid sequence of SEQ ID NO:169, and a light chain having an amino acid sequence of SEQ ID NO:102. In one aspect, provided herein is an antibody that binds PSMA, comprising a VH having an amino acid sequence having at least 95% identity to an amino acid sequence of SEQ ID NO:167. In one aspect, provided herein is an antibody that binds PSMA, comprising a VL having an amino acid sequence having at least 95% identity to an amino acid sequence of SEQ ID NO:100. In one aspect, provided herein is an antibody that binds PSMA, comprising a VH having an amino acid sequence having at least 95% identity to an amino acid sequence of SEQ ID NO:167, and a VL having an amino acid sequence having at least 95% identity to an amino acid sequence of SEQ ID NO:100. In one aspect, provided herein is an antibody that binds PSMA, comprising a heavy chain having an amino acid sequence having at least 95% identity to an amino acid sequence of SEQ ID NO:169. In one aspect, provided herein is an antibody that binds PSMA, comprising a light chain having an amino acid sequence having at least 95% identity to an amino acid sequence of SEQ ID NO:102. In one aspect, provided herein is an antibody that binds PSMA, comprising a heavy chain having an amino acid sequence having at least 95% identity to an amino acid sequence of SEQ ID NO:169, and a light chain having an amino acid sequence having at least 95% identity to an amino acid sequence of SEQ ID NO:102.

In one aspect, provided herein is an antibody that binds PSMA, comprising: (i) a VH comprising a VH CDR1, a VH CDR2, and a VH CDR3 having an amino acid sequence of SEQ ID NOs:137, 138, and 139, respectively, and (ii) a VL comprising a VL CDR1, VL CDR2, and VL CDR3 having an amino acid sequence of SEQ ID NOs:106, 107, and 108, respectively. In one aspect, provided herein is an antibody that binds PSMA, comprising: (i) a VH comprising a VH CDR1, a VH CDR2, and a VH CDR3 having an amino acid sequence of SEQ ID NOs:143, 144, and 145, respectively, and (ii) a VL comprising a VL CDR1, VL CDR2, and VL CDR3 having an amino acid sequence of SEQ ID NOs:112, 113, and 114, respectively. In one aspect, provided herein is an antibody that binds PSMA, comprising: (i) a VH comprising a VH CDR1, a VH CDR2, and a VH CDR3 having an amino acid sequence of SEQ ID NOs:149, 150, and 139, respectively, and (ii) a VL comprising a VL CDR1, VL CDR2, and VL CDR3 having an amino acid sequence of SEQ ID NOs:106, 107, and 108, respectively. In one aspect, provided herein is an antibody that binds PSMA, comprising: (i) a VH comprising a VH CDR1, a VH CDR2, and a VH CDR3 having an amino acid sequence of SEQ ID NOs:155, 156, and 157, respectively, and (ii) a VL comprising a VL CDR1, VL CDR2, and VL CDR3 having an amino acid sequence of SEQ ID NOs:124, 125, and 126, respectively. In one aspect, provided herein is an antibody that binds PSMA, comprising: (i) a VH comprising a VH CDR1, a VH CDR2, and a VH CDR3 having an amino acid sequence of SEQ ID NOs:161, 162, and 163, respectively, and (ii) a VL comprising a VL CDR1, VL CDR2, and VL CDR3 having an amino acid sequence of SEQ ID NOs:130, 113, and 108, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:167; and (ii) a VL comprising a VL CDR1, a VL CDR2, and a VL CDR3 having an amino acid sequence of a VL CDR1, a VL CDR2, and a VL CDR3, respectively, of SEQ ID NO:134. In one aspect, provided herein is an antibody that binds PSMA, comprising a VH having an amino acid sequence of SEQ ID NO:167. In one aspect, provided herein is an antibody that binds PSMA, comprising a VL having an amino acid sequence of SEQ ID NO:134. In one aspect, provided herein is an antibody that binds PSMA, comprising a VH having an amino acid sequence of SEQ ID NO:167, and a VL having an amino acid sequence of SEQ ID NO:134. In one aspect, provided herein is an antibody that binds PSMA, comprising a heavy chain having an amino acid sequence of SEQ ID NO:169. In one aspect, provided herein is an antibody that binds PSMA, comprising a light chain having an amino acid sequence of SEQ ID NO:136. In one aspect, provided herein is an antibody that binds PSMA, comprising a heavy chain having an amino acid sequence of SEQ ID NO:169, and a light chain having an amino acid sequence of SEQ ID NO:136. In one aspect, provided herein is an antibody that binds PSMA, comprising a VH having an amino acid sequence having at least 95% identity to an amino acid sequence of SEQ ID NO:167. In one aspect, provided herein is an antibody that binds PSMA, comprising a VL having an amino acid sequence having at least 95% identity to an amino acid sequence of SEQ ID NO:134. In one aspect, provided herein is an antibody that binds PSMA, comprising a VH having an amino acid sequence having at least 95% identity to an amino acid sequence of SEQ ID NO:167, and a VL having an amino acid sequence having at least 95% identity to an amino acid sequence of SEQ ID NO:134. In one aspect, provided herein is an antibody that binds PSMA, comprising a heavy chain having an amino acid sequence having at least 95% identity to an amino acid sequence of SEQ ID NO:169. In one aspect, provided herein is an antibody that binds PSMA, comprising a light chain having an amino acid sequence having at least 95% identity to an amino acid sequence of SEQ ID NO:136. In one aspect, provided herein is an antibody that binds PSMA, comprising a heavy chain having an amino acid sequence having at least 95% identity to an amino acid sequence of SEQ ID NO:169, and a light chain having an amino acid sequence having at least 95% identity to an amino acid sequence of SEQ ID NO:136.

In one aspect, provided herein is an antibody that binds PSMA, comprising: (i) a VH comprising a VH CDR1, a VH CDR2, and a VH CDR3 having an amino acid sequence of SEQ ID NOs:205, 206, and 207, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds PSMA, comprising: (i) a VH comprising a VH CDR1, a VH CDR2, and a VH CDR3 having an amino acid sequence of SEQ ID NOs:143, 212, and 213, respectively, and (ii) a VL comprising a VL CDR1, VL CDR2, and VL CDR3 having an amino acid sequence of SEQ ID NOs:214, 215, and 216, respectively. In one aspect, provided herein is an antibody that binds PSMA, comprising: (i) a VH comprising a VH CDR1, a VH CDR2, and a VH CDR3 having an amino acid sequence of SEQ ID NOs:217, 218, and 207, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds PSMA, comprising: (i) a VH comprising a VH CDR1, a VH CDR2, and a VH CDR3 having an amino acid sequence of SEQ ID NOs:223, 224, and 225, respectively, and (ii) a VL comprising a VL CDR1, VL CDR2, and VL CDR3 having an amino acid sequence of SEQ ID NOs:226, 227, and 228, respectively. In one aspect, provided herein is an antibody that binds PSMA, comprising: (i) a VH comprising a VH CDR1, a VH CDR2, and a VH CDR3 having an amino acid sequence of SEQ ID NOs:229, 230, and 231, respectively, and (ii) a VL comprising a VL CDR1, VL CDR2, and VL CDR3 having an amino acid sequence of SEQ ID NOs:232, 215, and 210, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:235; and (ii) a VL comprising a VL CDR1, a VL CDR2, and a VL CDR3 having an amino acid sequence of a VL CDR1, a VL CDR2, and a VL CDR3, respectively, of SEQ ID NO:236. In one aspect, provided herein is an antibody that binds PSMA, comprising a VH having an amino acid sequence of SEQ ID NO:235. In one aspect, provided herein is an antibody that binds PSMA, comprising a VL having an amino acid sequence of SEQ ID NO:236. In one aspect, provided herein is an antibody that binds PSMA, comprising a VH having an amino acid sequence of SEQ ID NO:235, and a VL having an amino acid sequence of SEQ ID NO:236. In one aspect, provided herein is an antibody that binds PSMA, comprising a heavy chain having an amino acid sequence of SEQ ID NO:237. In one aspect, provided herein is an antibody that binds PSMA, comprising a light chain having an amino acid sequence of SEQ ID NO:238. In one aspect, provided herein is an antibody that binds PSMA, comprising a heavy chain having an amino acid sequence of SEQ ID NO:237, and a light chain having an amino acid sequence of SEQ ID NO:238. In one aspect, provided herein is an antibody that binds PSMA, comprising a VH having an amino acid sequence having at least 95% identity to an amino acid sequence of SEQ ID NO:235. In one aspect, provided herein is an antibody that binds PSMA, comprising a VL having an amino acid sequence having at least 95% identity to an amino acid sequence of SEQ ID NO:236. In one aspect, provided herein is an antibody that binds PSMA, comprising a VH having an amino acid sequence having at least 95% identity to an amino acid sequence of SEQ ID NO:235, and a VL having an amino acid sequence having at least 95% identity to an amino acid sequence of SEQ ID NO:236. In one aspect, provided herein is an antibody that binds PSMA, comprising a heavy chain having an amino acid sequence having at least 95% identity to an amino acid sequence of SEQ ID NO:237. In one aspect, provided herein is an antibody that binds PSMA, comprising a light chain having an amino acid sequence having at least 95% identity to an amino acid sequence of SEQ ID NO:238. In one aspect, provided herein is an antibody that binds PSMA, comprising a heavy chain having an amino acid sequence having at least 95% identity to an amino acid sequence of SEQ ID NO:237, and a light chain having an amino acid sequence having at least 95% identity to an amino acid sequence of SEQ ID NO:238.

In one aspect, provided herein is an antibody that binds PSMA, comprising: (i) a VH comprising a VH CDR1, a VH CDR2, and a VH CDR3 having an amino acid sequence of SEQ ID NOs:205, 206, and 207, respectively, and (ii) a VL comprising a VL CDR1, VL CDR2, and VL CDR3 having an amino acid sequence of SEQ ID NOs:242, 209, and 244, respectively. In one aspect, provided herein is an antibody that binds PSMA, comprising: (i) a VH comprising a VH CDR1, a VH CDR2, and a VH CDR3 having an amino acid sequence of SEQ ID NOs:143, 212, and 213, respectively, and (ii) a VL comprising a VL CDR1, VL CDR2, and VL CDR3 having an amino acid sequence of SEQ ID NOs:248, 215, and 250, respectively. In one aspect, provided herein is an antibody that binds PSMA, comprising: (i) a VH comprising a VH CDR1, a VH CDR2, and a VH CDR3 having an amino acid sequence of SEQ ID NOs:217, 218, and 207, respectively, and (ii) a VL comprising a VL CDR1, VL CDR2, and VL CDR3 having an amino acid sequence of SEQ ID NOs:242, 209, and 244, respectively. In one aspect, provided herein is an antibody that binds PSMA, comprising: (i) a VH comprising a VH CDR1, a VH CDR2, and a VH CDR3 having an amino acid sequence of SEQ ID NOs:223, 224, and 225, respectively, and (ii) a VL comprising a VL CDR1, VL CDR2, and VL CDR3 having an amino acid sequence of SEQ ID NOs:260, 227, and 262, respectively. In one aspect, provided herein is an antibody that binds PSMA, comprising: (i) a VH comprising a VH CDR1, a VH CDR2, and a VH CDR3 having an amino acid sequence of SEQ ID NOs:229, 230, and 231, respectively, and (ii) a VL comprising a VL CDR1, VL CDR2, and VL CDR3 having an amino acid sequence of SEQ ID NOs:266, 215, and 244, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:235; and (ii) a VL comprising a VL CDR1, a VL CDR2, and a VL CDR3 having an amino acid sequence of a VL CDR1, a VL CDR2, and a VL CDR3, respectively, of SEQ ID NO:270. In one aspect, provided herein is an antibody that binds PSMA, comprising a VH having an amino acid sequence of SEQ ID NO:235. In one aspect, provided herein is an antibody that binds PSMA, comprising a VL having an amino acid sequence of SEQ ID NO:270. In one aspect, provided herein is an antibody that binds PSMA, comprising a VH having an amino acid sequence of SEQ ID NO:235, and a VL having an amino acid sequence of SEQ ID NO:270. In one aspect, provided herein is an antibody that binds PSMA, comprising a heavy chain having an amino acid sequence of SEQ ID NO:237. In one aspect, provided herein is an antibody that binds PSMA, comprising a light chain having an amino acid sequence of SEQ ID NO:272. In one aspect, provided herein is an antibody that binds PSMA, comprising a heavy chain having an amino acid sequence of SEQ ID NO:237, and a light chain having an amino acid sequence of SEQ ID NO:272. In one aspect, provided herein is an antibody that binds PSMA, comprising a VH having an amino acid sequence having at least 95% identity to an amino acid sequence of SEQ ID NO:235. In one aspect, provided herein is an antibody that binds PSMA, comprising a VL having an amino acid sequence having at least 95% identity to an amino acid sequence of SEQ ID NO:270. In one aspect, provided herein is an antibody that binds PSMA, comprising a VH having an amino acid sequence having at least 95% identity to an amino acid sequence of SEQ ID NO:235, and a VL having an amino acid sequence having at least 95% identity to an amino acid sequence of SEQ ID NO:270. In one aspect, provided herein is an antibody that binds PSMA, comprising a heavy chain having an amino acid sequence having at least 95% identity to an amino acid sequence of SEQ ID NO:237. In one aspect, provided herein is an antibody that binds PSMA, comprising a light chain having an amino acid sequence having at least 95% identity to an amino acid sequence of SEQ ID NO:272. In one aspect, provided herein is an antibody that binds PSMA, comprising a heavy chain having an amino acid sequence having at least 95% identity to an amino acid sequence of SEQ ID NO:237, and a light chain having an amino acid sequence having at least 95% identity to an amino acid sequence of SEQ ID NO:272.

In one aspect, provided herein is an antibody that binds PSMA, comprising: (i) a VH comprising a VH CDR1, a VH CDR2, and a VH CDR3 having an amino acid sequence of SEQ ID NOs:205, 274, and 207, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds PSMA, comprising: (i) a VH comprising a VH CDR1, a VH CDR2, and a VH CDR3 having an amino acid sequence of SEQ ID NOs:143, 280, and 213, respectively, and (ii) a VL comprising a VL CDR1, VL CDR2, and VL CDR3 having an amino acid sequence of SEQ ID NOs:214, 215, and 216, respectively. In one aspect, provided herein is an antibody that binds PSMA, comprising: (i) a VH comprising a VH CDR1, a VH CDR2, and a VH CDR3 having an amino acid sequence of SEQ ID NOs:217, 286, and 207, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds PSMA, comprising: (i) a VH comprising a VH CDR1, a VH CDR2, and a VH CDR3 having an amino acid sequence of SEQ ID NOs:223, 292, and 225, respectively, and (ii) a VL comprising a VL CDR1, VL CDR2, and VL CDR3 having an amino acid sequence of SEQ ID NOs:226, 227, and 228, respectively. In one aspect, provided herein is an antibody that binds PSMA, comprising: (i) a VH comprising a VH CDR1, a VH CDR2, and a VH CDR3 having an amino acid sequence of SEQ ID NOs:229, 298, and 231, respectively, and (ii) a VL comprising a VL CDR1, VL CDR2, and VL CDR3 having an amino acid sequence of SEQ ID NOs:232, 215, and 210, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:303; and (ii) a VL comprising a VL CDR1, a VL CDR2, and a VL CDR3 having an amino acid sequence of a VL CDR1, a VL CDR2, and a VL CDR3, respectively, of SEQ ID NO:236. In one aspect, provided herein is an antibody that binds PSMA, comprising a VH having an amino acid sequence of SEQ ID NO:303. In one aspect, provided herein is an antibody that binds PSMA, comprising a VL having an amino acid sequence of SEQ ID NO:236. In one aspect, provided herein is an antibody that binds PSMA, comprising a VH having an amino acid sequence of SEQ ID NO:303, and a VL having an amino acid sequence of SEQ ID NO:236. In one aspect, provided herein is an antibody that binds PSMA, comprising a heavy chain having an amino acid sequence of SEQ ID NO:305. In one aspect, provided herein is an antibody that binds PSMA, comprising a light chain having an amino acid sequence of SEQ ID NO:238. In one aspect, provided herein is an antibody that binds PSMA, comprising a heavy chain having an amino acid sequence of SEQ ID NO:305, and a light chain having an amino acid sequence of SEQ ID NO:238. In one aspect, provided herein is an antibody that binds PSMA, comprising a VH having an amino acid sequence having at least 95% identity to an amino acid sequence of SEQ ID NO:303. In one aspect, provided herein is an antibody that binds PSMA, comprising a VL having an amino acid sequence having at least 95% identity to an amino acid sequence of SEQ ID NO:236. In one aspect, provided herein is an antibody that binds PSMA, comprising a VH having an amino acid sequence having at least 95% identity to an amino acid sequence of SEQ ID NO:303, and a VL having an amino acid sequence having at least 95% identity to an amino acid sequence of SEQ ID NO:236. In one aspect, provided herein is an antibody that binds PSMA, comprising a heavy chain having an amino acid sequence having at least 95% identity to an amino acid sequence of SEQ ID NO:305. In one aspect, provided herein is an antibody that binds PSMA, comprising a light chain having an amino acid sequence having at least 95% identity to an amino acid sequence of SEQ ID NO:238. In one aspect, provided herein is an antibody that binds PSMA, comprising a heavy chain having an amino acid sequence having at least 95% identity to an amino acid sequence of SEQ ID NO:305, and a light chain having an amino acid sequence having at least 95% identity to an amino acid sequence of SEQ ID NO:238.

In one aspect, provided herein is an antibody that binds PSMA, comprising: (i) a VH comprising a VH CDR1, a VH CDR2, and a VH CDR3 having an amino acid sequence of SEQ ID NOs:205, 274, and 207, respectively, and (ii) a VL comprising a VL CDR1, VL CDR2, and VL CDR3 having an amino acid sequence of SEQ ID NOs:242, 209, and 244, respectively. In one aspect, provided herein is an antibody that binds PSMA, comprising: (i) a VH comprising a VH CDR1, a VH CDR2, and a VH CDR3 having an amino acid sequence of SEQ ID NOs:143, 280, and 213, respectively, and (ii) a VL comprising a VL CDR1, VL CDR2, and VL CDR3 having an amino acid sequence of SEQ ID NOs:248, 215, and 250, respectively. In one aspect, provided herein is an antibody that binds PSMA, comprising: (i) a VH comprising a VH CDR1, a VH CDR2, and a VH CDR3 having an amino acid sequence of SEQ ID NOs:217, 286, and 207, respectively, and (ii) a VL comprising a VL CDR1, VL CDR2, and VL CDR3 having an amino acid sequence of SEQ ID NOs:242, 209, and 244, respectively. In one aspect, provided herein is an antibody that binds PSMA, comprising: (i) a VH comprising a VH CDR1, a VH CDR2, and a VH CDR3 having an amino acid sequence of SEQ ID NOs:223, 292, and 225, respectively, and (ii) a VL comprising a VL CDR1, VL CDR2, and VL CDR3 having an amino acid sequence of SEQ ID NOs:260, 227, and 262, respectively. In one aspect, provided herein is an antibody that binds PSMA, comprising: (i) a VH comprising a VH CDR1, a VH CDR2, and a VH CDR3 having an amino acid sequence of SEQ ID NOs:229, 298, and 231, respectively, and (ii) a VL comprising a VL CDR1, VL CDR2, and VL CDR3 having an amino acid sequence of SEQ ID NOs:266, 215, and 244, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:303; and (ii) a VL comprising a VL CDR1, a VL CDR2, and a VL CDR3 having an amino acid sequence of a VL CDR1, a VL CDR2, and a VL CDR3, respectively, of SEQ ID NO:270. In one aspect, provided herein is an antibody that binds PSMA, comprising a VH having an amino acid sequence of SEQ ID NO:303. In one aspect, provided herein is an antibody that binds PSMA, comprising a VL having an amino acid sequence of SEQ ID NO:270. In one aspect, provided herein is an antibody that binds PSMA, comprising a VH having an amino acid sequence of SEQ ID NO:303, and a VL having an amino acid sequence of SEQ ID NO:270. In one aspect, provided herein is an antibody that binds PSMA, comprising a heavy chain having an amino acid sequence of SEQ ID NO:305. In one aspect, provided herein is an antibody that binds PSMA, comprising a light chain having an amino acid sequence of SEQ ID NO:272. In one aspect, provided herein is an antibody that binds PSMA, comprising a heavy chain having an amino acid sequence of SEQ ID NO:305, and a light chain having an amino acid sequence of SEQ ID NO:272. In one aspect, provided herein is an antibody that binds PSMA, comprising a VH having an amino acid sequence having at least 95% identity to an amino acid sequence of SEQ ID NO:303. In one aspect, provided herein is an antibody that binds PSMA, comprising a VL having an amino acid sequence having at least 95% identity to an amino acid sequence of SEQ ID NO:270. In one aspect, provided herein is an antibody that binds PSMA, comprising a VH having an amino acid sequence having at least 95% identity to an amino acid sequence of SEQ ID NO:303, and a VL having an amino acid sequence having at least 95% identity to an amino acid sequence of SEQ ID NO:270. In one aspect, provided herein is an antibody that binds PSMA, comprising a heavy chain having an amino acid sequence having at least 95% identity to an amino acid sequence of SEQ ID NO:305. In one aspect, provided herein is an antibody that binds PSMA, comprising a light chain having an amino acid sequence having at least 95% identity to an amino acid sequence of SEQ ID NO:272. In one aspect, provided herein is an antibody that binds PSMA, comprising a heavy chain having an amino acid sequence having at least 95% identity to an amino acid sequence of SEQ ID NO:305, and a light chain having an amino acid sequence having at least 95% identity to an amino acid sequence of SEQ ID NO:272.

In one aspect, provided herein is an antibody that binds PSMA, comprising: (i) a VH comprising a VH CDR1, a VH CDR2, and a VH CDR3 having an amino acid sequence of SEQ ID NOs:205, 342, and 343, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds PSMA, comprising: (i) a VH comprising a VH CDR1, a VH CDR2, and a VH CDR3 having an amino acid sequence of SEQ ID NOs:347, 348, and 213, respectively, and (ii) a VL comprising a VL CDR1, VL CDR2, and VL CDR3 having an amino acid sequence of SEQ ID NOs:214, 215, and 216, respectively. In one aspect, provided herein is an antibody that binds PSMA, comprising: (i) a VH comprising a VH CDR1, a VH CDR2, and a VH CDR3 having an amino acid sequence of SEQ ID NOs:353, 354, and 343, respectively, and (ii) a VL comprising a VL CDR1, VL CDR2, and VL CDR3 having an amino acid sequence of SEQ ID NOs:208, 209, and 210, respectively. In one aspect, provided herein is an antibody that binds PSMA, comprising: (i) a VH comprising a VH CDR1, a VH CDR2, and a VH CDR3 having an amino acid sequence of SEQ ID NOs:223, 360, and 225, respectively, and (ii) a VL comprising a VL CDR1, VL CDR2, and VL CDR3 having an amino acid sequence of SEQ ID NOs:226, 363, and 228, respectively. In one aspect, provided herein is an antibody that binds PSMA, comprising: (i) a VH comprising a VH CDR1, a VH CDR2, and a VH CDR3 having an amino acid sequence of SEQ ID NOs:365, 366, and 367, respectively, and (ii) a VL comprising a VL CDR1, VL CDR2, and VL CDR3 having an amino acid sequence of SEQ ID NOs:232, 215, and 210, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:371; and (ii) a VL comprising a VL CDR1, a VL CDR2, and a VL CDR3 having an amino acid sequence of a VL CDR1, a VL CDR2, and a VL CDR3, respectively, of SEQ ID NO:372. In one aspect, provided herein is an antibody that binds PSMA, comprising a VH having an amino acid sequence of SEQ ID NO:371. In one aspect, provided herein is an antibody that binds PSMA, comprising a VL having an amino acid sequence of SEQ ID NO:372. In one aspect, provided herein is an antibody that binds PSMA, comprising a VH having an amino acid sequence of SEQ ID NO:371, and a VL having an amino acid sequence of SEQ ID NO:372. In one aspect, provided herein is an antibody that binds PSMA, comprising a heavy chain having an amino acid sequence of SEQ ID NO:373. In one aspect, provided herein is an antibody that binds PSMA, comprising a light chain having an amino acid sequence of SEQ ID NO:374. In one aspect, provided herein is an antibody that binds PSMA, comprising a heavy chain having an amino acid sequence of SEQ ID NO:373, and a light chain having an amino acid sequence of SEQ ID NO:374. In one aspect, provided herein is an antibody that binds PSMA, comprising a VH having an amino acid sequence having at least 95% identity to an amino acid sequence of SEQ ID NO:371. In one aspect, provided herein is an antibody that binds PSMA, comprising a VL having an amino acid sequence having at least 95% identity to an amino acid sequence of SEQ ID NO:372. In one aspect, provided herein is an antibody that binds PSMA, comprising a VH having an amino acid sequence having at least 95% identity to an amino acid sequence of SEQ ID NO:371, and a VL having an amino acid sequence having at least 95% identity to an amino acid sequence of SEQ ID NO:372. In one aspect, provided herein is an antibody that binds PSMA, comprising a heavy chain having an amino acid sequence having at least 95% identity to an amino acid sequence of SEQ ID NO:373. In one aspect, provided herein is an antibody that binds PSMA, comprising a light chain having an amino acid sequence having at least 95% identity to an amino acid sequence of SEQ ID NO:374. In one aspect, provided herein is an antibody that binds PSMA, comprising a heavy chain having an amino acid sequence having at least 95% identity to an amino acid sequence of SEQ ID NO:373, and a light chain having an amino acid sequence having at least 95% identity to an amino acid sequence of SEQ ID NO:374.

In one aspect, provided herein is an antibody that binds PSMA, comprising: (i) a VH comprising a VH CDR1, a VH CDR2, and a VH CDR3 having an amino acid sequence of SEQ ID NOs:375, 376, and 377, respectively, and (ii) a VL comprising a VL CDR1, VL CDR2, and VL CDR3 having an amino acid sequence of SEQ ID NOs:378, 379, and 380, respectively. In one aspect, provided herein is an antibody that binds PSMA, comprising: (i) a VH comprising a VH CDR1, a VH CDR2, and a VH CDR3 having an amino acid sequence of SEQ ID NOs:381, 382, and 383, respectively, and (ii) a VL comprising a VL CDR1, VL CDR2, and VL CDR3 having an amino acid sequence of SEQ ID NOs:384, 385, and 386, respectively. In one aspect, provided herein is an antibody that binds PSMA, comprising: (i) a VH comprising a VH CDR1, a VH CDR2, and a VH CDR3 having an amino acid sequence of SEQ ID NOs:387, 388, and 377, respectively, and (ii) a VL comprising a VL CDR1, VL CDR2, and VL CDR3 having an amino acid sequence of SEQ ID NOs:378, 379, and 380, respectively. In one aspect, provided herein is an antibody that binds PSMA, comprising: (i) a VH comprising a VH CDR1, a VH CDR2, and a VH CDR3 having an amino acid sequence of SEQ ID NOs:393, 394, and 395, respectively, and (ii) a VL comprising a VL CDR1, VL CDR2, and VL CDR3 having an amino acid sequence of SEQ ID NOs:396, 397, and 398, respectively. In one aspect, provided herein is an antibody that binds PSMA, comprising: (i) a VH comprising a VH CDR1, a VH CDR2, and a VH CDR3 having an amino acid sequence of SEQ ID NOs:399, 400, and 401, respectively, and (ii) a VL comprising a VL CDR1, VL CDR2, and VL CDR3 having an amino acid sequence of SEQ ID NOs:402, 385, and 380, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:405; and (ii) a VL comprising a VL CDR1, a VL CDR2, and a VL CDR3 having an amino acid sequence of a VL CDR1, a VL CDR2, and a VL CDR3, respectively, of SEQ ID NO:406. In one aspect, provided herein is an antibody that binds PSMA, comprising a VH having an amino acid sequence of SEQ ID NO:405. In one aspect, provided herein is an antibody that binds PSMA, comprising a VL having an amino acid sequence of SEQ ID NO:406. In one aspect, provided herein is an antibody that binds PSMA, comprising a VH having an amino acid sequence of SEQ ID NO:405, and a VL having an amino acid sequence of SEQ ID NO:406. In one aspect, provided herein is an antibody that binds PSMA, comprising a heavy chain having an amino acid sequence of SEQ ID NO:407. In one aspect, provided herein is an antibody that binds PSMA, comprising a light chain having an amino acid sequence of SEQ ID NO:408. In one aspect, provided herein is an antibody that binds PSMA, comprising a heavy chain having an amino acid sequence of SEQ ID NO:407, and a light chain having an amino acid sequence of SEQ ID NO:408. In one aspect, provided herein is an antibody that binds PSMA, comprising a VH having an amino acid sequence having at least 95% identity to an amino acid sequence of SEQ ID NO:405. In one aspect, provided herein is an antibody that binds PSMA, comprising a VL having an amino acid sequence having at least 95% identity to an amino acid sequence of SEQ ID NO:406. In one aspect, provided herein is an antibody that binds PSMA, comprising a VH having an amino acid sequence having at least 95% identity to an amino acid sequence of SEQ ID NO:405, and a VL having an amino acid sequence having at least 95% identity to an amino acid sequence of SEQ ID NO:406. In one aspect, provided herein is an antibody that binds PSMA, comprising a heavy chain having an amino acid sequence having at least 95% identity to an amino acid sequence of SEQ ID NO:407. In one aspect, provided herein is an antibody that binds PSMA, comprising a light chain having an amino acid sequence having at least 95% identity to an amino acid sequence of SEQ ID NO:408. In one aspect, provided herein is an antibody that binds PSMA, comprising a heavy chain having an amino acid sequence having at least 95% identity to an amino acid sequence of SEQ ID NO:407, and a light chain having an amino acid sequence having at least 95% identity to an amino acid sequence of SEQ ID NO:408.

In one aspect, provided herein is an antibody that binds PSMA, comprising: (i) a VH comprising a VH CDR1, a VH CDR2, and a VH CDR3 having an amino acid sequence of SEQ ID NOs:205, 206, and 411, respectively, and (ii) a VL comprising a VL CDR1, VL CDR2, and VL CDR3 having an amino acid sequence of SEQ ID NOs:242, 209, and 244, respectively. In one aspect, provided herein is an antibody that binds PSMA, comprising: (i) a VH comprising a VH CDR1, a VH CDR2, and a VH CDR3 having an amino acid sequence of SEQ ID NOs:143, 212, and 417, respectively, and (ii) a VL comprising a VL CDR1, VL CDR2, and VL CDR3 having an amino acid sequence of SEQ ID NOs:248, 215, and 250, respectively. In one aspect, provided herein is an antibody that binds PSMA, comprising: (i) a VH comprising a VH CDR1, a VH CDR2, and a VH CDR3 having an amino acid sequence of SEQ ID NOs:217, 218, and 411, respectively, and (ii) a VL comprising a VL CDR1, VL CDR2, and VL CDR3 having an amino acid sequence of SEQ ID NOs:242, 209, and 244, respectively. In one aspect, provided herein is an antibody that binds PSMA, comprising: (i) a VH comprising a VH CDR1, a VH CDR2, and a VH CDR3 having an amino acid sequence of SEQ ID NOs:223, 224, and 429, respectively, and (ii) a VL comprising a VL CDR1, VL CDR2, and VL CDR3 having an amino acid sequence of SEQ ID NOs:260, 227, and 262, respectively. In one aspect, provided herein is an antibody that binds PSMA, comprising: (i) a VH comprising a VH CDR1, a VH CDR2, and a VH CDR3 having an amino acid sequence of SEQ ID NOs:229, 230, and 435, respectively, and (ii) a VL comprising a VL CDR1, VL CDR2, and VL CDR3 having an amino acid sequence of SEQ ID NOs:266, 215, and 244, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:439; and (ii) a VL comprising a VL CDR1, a VL CDR2, and a VL CDR3 having an amino acid sequence of a VL CDR1, a VL CDR2, and a VL CDR3, respectively, of SEQ ID NO:270. In one aspect, provided herein is an antibody that binds PSMA, comprising a VH having an amino acid sequence of SEQ ID NO:439. In one aspect, provided herein is an antibody that binds PSMA, comprising a VL having an amino acid sequence of SEQ ID NO:270. In one aspect, provided herein is an antibody that binds PSMA, comprising a VH having an amino acid sequence of SEQ ID NO:439, and a VL having an amino acid sequence of SEQ ID NO:270. In one aspect, provided herein is an antibody that binds PSMA, comprising a heavy chain having an amino acid sequence of SEQ ID NO:441. In one aspect, provided herein is an antibody that binds PSMA, comprising a light chain having an amino acid sequence of SEQ ID NO:272. In one aspect, provided herein is an antibody that binds PSMA, comprising a heavy chain having an amino acid sequence of SEQ ID NO:441, and a light chain having an amino acid sequence of SEQ ID NO:272. In one aspect, provided herein is an antibody that binds PSMA, comprising a VH having an amino acid sequence having at least 95% identity to an amino acid sequence of SEQ ID NO:439. In one aspect, provided herein is an antibody that binds PSMA, comprising a VL having an amino acid sequence having at least 95% identity to an amino acid sequence of SEQ ID NO:270. In one aspect, provided herein is an antibody that binds PSMA, comprising a VH having an amino acid sequence having at least 95% identity to an amino acid sequence of SEQ ID NO:439, and a VL having an amino acid sequence having at least 95% identity to an amino acid sequence of SEQ ID NO:270. In one aspect, provided herein is an antibody that binds PSMA, comprising a heavy chain having an amino acid sequence having at least 95% identity to an amino acid sequence of SEQ ID NO:441. In one aspect, provided herein is an antibody that binds PSMA, comprising a light chain having an amino acid sequence having at least 95% identity to an amino acid sequence of SEQ ID NO:272. In one aspect, provided herein is an antibody that binds PSMA, comprising a heavy chain having an amino acid sequence having at least 95% identity to an amino acid sequence of SEQ ID NO:441, and a light chain having an amino acid sequence having at least 95% identity to an amino acid sequence of SEQ ID NO:272.

In one aspect, provided herein is an antibody that binds PSMA, comprising: (i) a VH comprising a VH CDR1, a VH CDR2, and a VH CDR3 having an amino acid sequence of SEQ ID NOs:443, 444, and 445, respectively, and (ii) a VL comprising a VL CDR1, VL CDR2, and VL CDR3 having an amino acid sequence of SEQ ID NOs:446, 447, and 448, respectively. In one aspect, provided herein is an antibody that binds PSMA, comprising: (i) a VH comprising a VH CDR1, a VH CDR2, and a VH CDR3 having an amino acid sequence of SEQ ID NOs:143, 450, and 451, respectively, and (ii) a VL comprising a VL CDR1, VL CDR2, and VL CDR3 having an amino acid sequence of SEQ ID NOs:452, 453, and 454, respectively. In one aspect, provided herein is an antibody that binds PSMA, comprising: (i) a VH comprising a VH CDR1, a VH CDR2, and a VH CDR3 having an amino acid sequence of SEQ ID NOs:455, 456, and 445, respectively, and (ii) a VL comprising a VL CDR1, VL CDR2, and VL CDR3 having an amino acid sequence of SEQ ID NOs:446, 447, and 448, respectively. In one aspect, provided herein is an antibody that binds PSMA, comprising: (i) a VH comprising a VH CDR1, a VH CDR2, and a VH CDR3 having an amino acid sequence of SEQ ID NOs:461, 462, and 463, respectively, and (ii) a VL comprising a VL CDR1, VL CDR2, and VL CDR3 having an amino acid sequence of SEQ ID NOs:464, 465, and 466, respectively. In one aspect, provided herein is an antibody that binds PSMA, comprising: (i) a VH comprising a VH CDR1, a VH CDR2, and a VH CDR3 having an amino acid sequence of SEQ ID NOs:467, 468, and 469, respectively, and (ii) a VL comprising a VL CDR1, VL CDR2, and VL CDR3 having an amino acid sequence of SEQ ID NOs:470, 453, and 448, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:473; and (ii) a VL comprising a VL CDR1, a VL CDR2, and a VL CDR3 having an amino acid sequence of a VL CDR1, a VL CDR2, and a VL CDR3, respectively, of SEQ ID NO:474. In one aspect, provided herein is an antibody that binds PSMA, comprising a VH having an amino acid sequence of SEQ ID NO:473. In one aspect, provided herein is an antibody that binds PSMA, comprising a VL having an amino acid sequence of SEQ ID NO:474. In one aspect, provided herein is an antibody that binds PSMA, comprising a VH having an amino acid sequence of SEQ ID NO:473, and a VL having an amino acid sequence of SEQ ID NO:474. In one aspect, provided herein is an antibody that binds PSMA, comprising a heavy chain having an amino acid sequence of SEQ ID NO:475. In one aspect, provided herein is an antibody that binds PSMA, comprising a light chain having an amino acid sequence of SEQ ID NO:476. In one aspect, provided herein is an antibody that binds PSMA, comprising a heavy chain having an amino acid sequence of SEQ ID NO:475, and a light chain having an amino acid sequence of SEQ ID NO:476. In one aspect, provided herein is an antibody that binds PSMA, comprising a VH having an amino acid sequence having at least 95% identity to an amino acid sequence of SEQ ID NO:473. In one aspect, provided herein is an antibody that binds PSMA, comprising a VL having an amino acid sequence having at least 95% identity to an amino acid sequence of SEQ ID NO:474. In one aspect, provided herein is an antibody that binds PSMA, comprising a VH having an amino acid sequence having at least 95% identity to an amino acid sequence of SEQ ID NO:473, and a VL having an amino acid sequence having at least 95% identity to an amino acid sequence of SEQ ID NO:474. In one aspect, provided herein is an antibody that binds PSMA, comprising a heavy chain having an amino acid sequence having at least 95% identity to an amino acid sequence of SEQ ID NO:475. In one aspect, provided herein is an antibody that binds PSMA, comprising a light chain having an amino acid sequence having at least 95% identity to an amino acid sequence of SEQ ID NO:476. In one aspect, provided herein is an antibody that binds PSMA, comprising a heavy chain having an amino acid sequence having at least 95% identity to an amino acid sequence of SEQ ID NO:475, and a light chain having an amino acid sequence having at least 95% identity to an amino acid sequence of SEQ ID NO:476.

In one aspect, provided herein is an antibody that binds PSMA, comprising: (i) a VH comprising a VH CDR1, a VH CDR2, and a VH CDR3 having an amino acid sequence of SEQ ID NOs:477, 478, and 479, respectively, and (ii) a VL comprising a VL CDR1, VL CDR2, and VL CDR3 having an amino acid sequence of SEQ ID NOs:480, 481, and 482, respectively. In one aspect, provided herein is an antibody that binds PSMA, comprising: (i) a VH comprising a VH CDR1, a VH CDR2, and a VH CDR3 having an amino acid sequence of SEQ ID NOs:483, 484, and 485, respectively, and (ii) a VL comprising a VL CDR1, VL CDR2, and VL CDR3 having an amino acid sequence of SEQ ID NOs:486, 487, and 488, respectively. In one aspect, provided herein is an antibody that binds PSMA, comprising: (i) a VH comprising a VH CDR1, a VH CDR2, and a VH CDR3 having an amino acid sequence of SEQ ID NOs:489, 490, and 479, respectively, and (ii) a VL comprising a VL CDR1, VL CDR2, and VL CDR3 having an amino acid sequence of SEQ ID NOs:480, 481, and 482, respectively. In one aspect, provided herein is an antibody that binds PSMA, comprising: (i) a VH comprising a VH CDR1, a VH CDR2, and a VH CDR3 having an amino acid sequence of SEQ ID NOs:495, 496, and 497, respectively, and (ii) a VL comprising a VL CDR1, VL CDR2, and VL CDR3 having an amino acid sequence of SEQ ID NOs:498, 499, and 500, respectively. In one aspect, provided herein is an antibody that binds PSMA, comprising: (i) a VH comprising a VH CDR1, a VH CDR2, and a VH CDR3 having an amino acid sequence of SEQ ID NOs:501, 502, and 503, respectively, and (ii) a VL comprising a VL CDR1, VL CDR2, and VL CDR3 having an amino acid sequence of SEQ ID NOs:504, 487, and 482, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:507; and (ii) a VL comprising a VL CDR1, a VL CDR2, and a VL CDR3 having an amino acid sequence of a VL CDR1, a VL CDR2, and a VL CDR3, respectively, of SEQ ID NO:508. In one aspect, provided herein is an antibody that binds PSMA, comprising a VH having an amino acid sequence of SEQ ID NO:507. In one aspect, provided herein is an antibody that binds PSMA, comprising a VL having an amino acid sequence of SEQ ID NO:508. In one aspect, provided herein is an antibody that binds PSMA, comprising a VH having an amino acid sequence of SEQ ID NO:507, and a VL having an amino acid sequence of SEQ ID NO:508. In one aspect, provided herein is an antibody that binds PSMA, comprising a heavy chain having an amino acid sequence of SEQ ID NO:509. In one aspect, provided herein is an antibody that binds PSMA, comprising a light chain having an amino acid sequence of SEQ ID NO:510. In one aspect, provided herein is an antibody that binds PSMA, comprising a heavy chain having an amino acid sequence of SEQ ID NO:509, and a light chain having an amino acid sequence of SEQ ID NO:510. In one aspect, provided herein is an antibody that binds PSMA, comprising a VH having an amino acid sequence having at least 95% identity to an amino acid sequence of SEQ ID NO:507. In one aspect, provided herein is an antibody that binds PSMA, comprising a VL having an amino acid sequence having at least 95% identity to an amino acid sequence of SEQ ID NO:508. In one aspect, provided herein is an antibody that binds PSMA, comprising a VH having an amino acid sequence having at least 95% identity to an amino acid sequence of SEQ ID NO:507, and a VL having an amino acid sequence having at least 95% identity to an amino acid sequence of SEQ ID NO:508. In one aspect, provided herein is an antibody that binds PSMA, comprising a heavy chain having an amino acid sequence having at least 95% identity to an amino acid sequence of SEQ ID NO:509. In one aspect, provided herein is an antibody that binds PSMA, comprising a light chain having an amino acid sequence having at least 95% identity to an amino acid sequence of SEQ ID NO:510. In one aspect, provided herein is an antibody that binds PSMA, comprising a heavy chain having an amino acid sequence having at least 95% identity to an amino acid sequence of SEQ ID NO:509, and a light chain having an amino acid sequence having at least 95% identity to an amino acid sequence of SEQ ID NO:510.

In one aspect, provided herein is an antibody that binds PSMA, comprising: (i) a VH comprising a VH CDR1, a VH CDR2, and a VH CDR3 having an amino acid sequence of SEQ ID NOs:69, 512, and 513, respectively, and (ii) a VL comprising a VL CDR1, VL CDR2, and VL CDR3 having an amino acid sequence of SEQ ID NOs:514, 515, and 516, respectively. In one aspect, provided herein is an antibody that binds PSMA, comprising: (i) a VH comprising a VH CDR1, a VH CDR2, and a VH CDR3 having an amino acid sequence of SEQ ID NOs:517, 518, and 519, respectively, and (ii) a VL comprising a VL CDR1, VL CDR2, and VL CDR3 having an amino acid sequence of SEQ ID NOs:520, 385, and 522, respectively. In one aspect, provided herein is an antibody that binds PSMA, comprising: (i) a VH comprising a VH CDR1, a VH CDR2, and a VH CDR3 having an amino acid sequence of SEQ ID NOs:523, 524, and 513, respectively, and (ii) a VL comprising a VL CDR1, VL CDR2, and VL CDR3 having an amino acid sequence of SEQ ID NOs:514, 515, and 516, respectively. In one aspect, provided herein is an antibody that binds PSMA, comprising: (i) a VH comprising a VH CDR1, a VH CDR2, and a VH CDR3 having an amino acid sequence of SEQ ID NOs:529, 530, and 531, respectively, and (ii) a VL comprising a VL CDR1, VL CDR2, and VL CDR3 having an amino acid sequence of SEQ ID NOs:532, 533, and 534, respectively. In one aspect, provided herein is an antibody that binds PSMA, comprising: (i) a VH comprising a VH CDR1, a VH CDR2, and a VH CDR3 having an amino acid sequence of SEQ ID NOs:535, 536, and 537, respectively, and (ii) a VL comprising a VL CDR1, VL CDR2, and VL CDR3 having an amino acid sequence of SEQ ID NOs:538, 385, and 516, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:541; and (ii) a VL comprising a VL CDR1, a VL CDR2, and a VL CDR3 having an amino acid sequence of a VL CDR1, a VL CDR2, and a VL CDR3, respectively, of SEQ ID NO:542. In one aspect, provided herein is an antibody that binds PSMA, comprising a VH having an amino acid sequence of SEQ ID NO:541. In one aspect, provided herein is an antibody that binds PSMA, comprising a VL having an amino acid sequence of SEQ ID NO:542. In one aspect, provided herein is an antibody that binds PSMA, comprising a VH having an amino acid sequence of SEQ ID NO:541, and a VL having an amino acid sequence of SEQ ID NO:542. In one aspect, provided herein is an antibody that binds PSMA, comprising a heavy chain having an amino acid sequence of SEQ ID NO:543. In one aspect, provided herein is an antibody that binds PSMA, comprising a light chain having an amino acid sequence of SEQ ID NO:544. In one aspect, provided herein is an antibody that binds PSMA, comprising a heavy chain having an amino acid sequence of SEQ ID NO:543, and a light chain having an amino acid sequence of SEQ ID NO:544. In one aspect, provided herein is an antibody that binds PSMA, comprising a VH having an amino acid sequence having at least 95% identity to an amino acid sequence of SEQ ID NO:541. In one aspect, provided herein is an antibody that binds PSMA, comprising a VL having an amino acid sequence having at least 95% identity to an amino acid sequence of SEQ ID NO:542. In one aspect, provided herein is an antibody that binds PSMA, comprising a VH having an amino acid sequence having at least 95% identity to an amino acid sequence of SEQ ID NO:541, and a VL having an amino acid sequence having at least 95% identity to an amino acid sequence of SEQ ID NO:542. In one aspect, provided herein is an antibody that binds PSMA, comprising a heavy chain having an amino acid sequence having at least 95% identity to an amino acid sequence of SEQ ID NO:543. In one aspect, provided herein is an antibody that binds PSMA, comprising a light chain having an amino acid sequence having at least 95% identity to an amino acid sequence of SEQ ID NO:544. In one aspect, provided herein is an antibody that binds PSMA, comprising a heavy chain having an amino acid sequence having at least 95% identity to an amino acid sequence of SEQ ID NO:543, and a light chain having an amino acid sequence having at least 95% identity to an amino acid sequence of SEQ ID NO:544.

In one aspect, provided herein is an antibody that binds PSMA, comprising: (i) a VH comprising a VH CDR1, a VH CDR2, and a VH CDR3 having an amino acid sequence of SEQ ID NOs:545, 546, and 547, respectively, and (ii) a VL comprising a VL CDR1, VL CDR2, and VL CDR3 having an amino acid sequence of SEQ ID NOs:548,549, and 550, respectively. In one aspect, provided herein is an antibody that binds PSMA, comprising: (i) a VH comprising a VH CDR1, a VH CDR2, and a VH CDR3 having an amino acid sequence of SEQ ID NOs:143, 518, and 553, respectively, and (ii) a VL comprising a VL CDR1, VL CDR2, and VL CDR3 having an amino acid sequence of SEQ ID NOs:554,555, and 556, respectively. In one aspect, provided herein is an antibody that binds PSMA, comprising: (i) a VH comprising a VH CDR1, a VH CDR2, and a VH CDR3 having an amino acid sequence of SEQ ID NOs:557, 558, and 547, respectively, and (ii) a VL comprising a VL CDR1, VL CDR2, and VL CDR3 having an amino acid sequence of SEQ ID NOs:548, 549, and 550, respectively. In one aspect, provided herein is an antibody that binds PSMA, comprising: (i) a VH comprising a VH CDR1, a VH CDR2, and a VH CDR3 having an amino acid sequence of SEQ ID NOs:563, 564, and 565, respectively, and (ii) a VL comprising a VL CDR1, VL CDR2, and VL CDR3 having an amino acid sequence of SEQ ID NOs:566, 567, and 568, respectively. In one aspect, provided herein is an antibody that binds PSMA, comprising: (i) a VH comprising a VH CDR1, a VH CDR2, and a VH CDR3 having an amino acid sequence of SEQ ID NOs:161, 570, and 571, respectively, and (ii) a VL comprising a VL CDR1, VL CDR2, and VL CDR3 having an amino acid sequence of SEQ ID NOs:572, 555, and 550, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:575; and (ii) a VL comprising a VL CDR1, a VL CDR2, and a VL CDR3 having an amino acid sequence of a VL CDR1, a VL CDR2, and a VL CDR3, respectively, of SEQ ID NO:576. In one aspect, provided herein is an antibody that binds PSMA, comprising a VH having an amino acid sequence of SEQ ID NO:575. In one aspect, provided herein is an antibody that binds PSMA, comprising a VL having an amino acid sequence of SEQ ID NO:576. In one aspect, provided herein is an antibody that binds PSMA, comprising a VH having an amino acid sequence of SEQ ID NO:575, and a VL having an amino acid sequence of SEQ ID NO:576. In one aspect, provided herein is an antibody that binds PSMA, comprising a heavy chain having an amino acid sequence of SEQ ID NO:577. In one aspect, provided herein is an antibody that binds PSMA, comprising a light chain having an amino acid sequence of SEQ ID NO:578. In one aspect, provided herein is an antibody that binds PSMA, comprising a heavy chain having an amino acid sequence of SEQ ID NO:577, and a light chain having an amino acid sequence of SEQ ID NO:578. In one aspect, provided herein is an antibody that binds PSMA, comprising a VH having an amino acid sequence having at least 95% identity to an amino acid sequence of SEQ ID NO:575. In one aspect, provided herein is an antibody that binds PSMA, comprising a VL having an amino acid sequence having at least 95% identity to an amino acid sequence of SEQ ID NO:576. In one aspect, provided herein is an antibody that binds PSMA, comprising a VH having an amino acid sequence having at least 95% identity to an amino acid sequence of SEQ ID NO:575, and a VL having an amino acid sequence having at least 95% identity to an amino acid sequence of SEQ ID NO:576. In one aspect, provided herein is an antibody that binds PSMA, comprising a heavy chain having an amino acid sequence having at least 95% identity to an amino acid sequence of SEQ ID NO:577. In one aspect, provided herein is an antibody that binds PSMA, comprising a light chain having an amino acid sequence having at least 95% identity to an amino acid sequence of SEQ ID NO:578. In one aspect, provided herein is an antibody that binds PSMA, comprising a heavy chain having an amino acid sequence having at least 95% identity to an amino acid sequence of SEQ ID NO:577, and a light chain having an amino acid sequence having at least 95% identity to an amino acid sequence of SEQ ID NO:578.

In one aspect, provided herein is an antibody that binds PSMA, comprising: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds PSMA, comprising: (i) a VH comprising a VH CDR1, a VH CDR2, and a VH CDR3 having an amino acid sequence of SEQ ID NOs:75, 76, and 587, respectively, and (ii) a VL comprising a VL CDR1, VL CDR2, and VL CDR3 having an amino acid sequence of SEQ ID NOs:78, 79, and 80, respectively. In one aspect, provided herein is an antibody that binds PSMA, comprising: (i) a VH comprising a VH CDR1, a VH CDR2, and a VH CDR3 having an amino acid sequence of SEQ ID NOs:81, 82, and 71, respectively, and (ii) a VL comprising a VL CDR1, VL CDR2, and VL CDR3 having an amino acid sequence of SEQ ID NOs:72, 73, and 74, respectively. In one aspect, provided herein is an antibody that binds PSMA, comprising: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:90, 91, and 92, respectively. In one aspect, provided herein is an antibody that binds PSMA, comprising: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:96, 79, and 74, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In one aspect, provided herein is an antibody that binds PSMA, comprising a VH having an amino acid sequence of SEQ ID NO:99. In one aspect, provided herein is an antibody that binds PSMA, comprising a VL having an amino acid sequence of SEQ ID NO:100. In one aspect, provided herein is an antibody that binds PSMA, comprising a VH having an amino acid sequence of SEQ ID NO:99, and a VL having an amino acid sequence of SEQ ID NO:100. In one aspect, provided herein is an antibody that binds PSMA, comprising a heavy chain having an amino acid sequence of SEQ ID NO:611. In one aspect, provided herein is an antibody that binds PSMA, comprising a light chain having an amino acid sequence of SEQ ID NO:612. In one aspect, provided herein is an antibody that binds PSMA, comprising a heavy chain having an amino acid sequence of SEQ ID NO:611, and a light chain having an amino acid sequence of SEQ ID NO:612. In one aspect, provided herein is an antibody that binds PSMA, comprising a VH having an amino acid sequence having at least 95% identity to an amino acid sequence of SEQ ID NO:99. In one aspect, provided herein is an antibody that binds PSMA, comprising a VL having an amino acid sequence having at least 95% identity to an amino acid sequence of SEQ ID NO:100. In one aspect, provided herein is an antibody that binds PSMA, comprising a VH having an amino acid sequence having at least 95% identity to an amino acid sequence of SEQ ID NO:99, and a VL having an amino acid sequence having at least 95% identity to an amino acid sequence of SEQ ID NO:100. In one aspect, provided herein is an antibody that binds PSMA, comprising a heavy chain having an amino acid sequence having at least 95% identity to an amino acid sequence of SEQ ID NO:611. In one aspect, provided herein is an antibody that binds PSMA, comprising a light chain having an amino acid sequence having at least 95% identity to an amino acid sequence of SEQ ID NO:612. In one aspect, provided herein is an antibody that binds PSMA, comprising a heavy chain having an amino acid sequence having at least 95% identity to an amino acid sequence of SEQ ID NO:611, and a light chain having an amino acid sequence having at least 95% identity to an amino acid sequence of SEQ ID NO:612.

In one aspect, provided herein is an antibody that binds PSMA, comprising: (i) a VH comprising a VH CDR1, a VH CDR2, and a VH CDR3 having an amino acid sequence of SEQ ID NOs:477, 478, and 615, respectively, and (ii) a VL comprising a VL CDR1, VL CDR2, and VL CDR3 having an amino acid sequence of SEQ ID NOs:480, 481, and 482, respectively. In one aspect, provided herein is an antibody that binds PSMA, comprising: (i) a VH comprising a VH CDR1, a VH CDR2, and a VH CDR3 having an amino acid sequence of SEQ ID NOs:483, 484, and 621, respectively, and (ii) a VL comprising a VL CDR1, VL CDR2, and VL CDR3 having an amino acid sequence of SEQ ID NOs:486, 523, and 488, respectively. In one aspect, provided herein is an antibody that binds PSMA, comprising: (i) a VH comprising a VH CDR1, a VH CDR2, and a VH CDR3 having an amino acid sequence of SEQ ID NOs:489, 490, and 615, respectively, and (ii) a VL comprising a VL CDR1, VL CDR2, and VL CDR3 having an amino acid sequence of SEQ ID NOs:480, 481, and 482, respectively. In one aspect, provided herein is an antibody that binds PSMA, comprising: (i) a VH comprising a VH CDR1, a VH CDR2, and a VH CDR3 having an amino acid sequence of SEQ ID NOs:495, 496, and 633, respectively, and (ii) a VL comprising a VL CDR1, VL CDR2, and VL CDR3 having an amino acid sequence of SEQ ID NOs:498, 499, and 500, respectively. In one aspect, provided herein is an antibody that binds PSMA, comprising: (i) a VH comprising a VH CDR1, a VH CDR2, and a VH CDR3 having an amino acid sequence of SEQ ID NOs:501, 502, and 639, respectively, and (ii) a VL comprising a VL CDR1, VL CDR2, and VL CDR3 having an amino acid sequence of SEQ ID NOs:504, 487, and 482, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:643; and (ii) a VL comprising a VL CDR1, a VL CDR2, and a VL CDR3 having an amino acid sequence of a VL CDR1, a VL CDR2, and a VL CDR3, respectively, of SEQ ID NO:508. In one aspect, provided herein is an antibody that binds PSMA, comprising a VH having an amino acid sequence of SEQ ID NO:643. In one aspect, provided herein is an antibody that binds PSMA, comprising a VL having an amino acid sequence of SEQ ID NO:508. In one aspect, provided herein is an antibody that binds PSMA, comprising a VH having an amino acid sequence of SEQ ID NO:643, and a VL having an amino acid sequence of SEQ ID NO:508. In one aspect, provided herein is an antibody that binds PSMA, comprising a heavy chain having an amino acid sequence of SEQ ID NO:645. In one aspect, provided herein is an antibody that binds PSMA, comprising a light chain having an amino acid sequence of SEQ ID NO:646. In one aspect, provided herein is an antibody that binds PSMA, comprising a heavy chain having an amino acid sequence of SEQ ID NO:645, and a light chain having an amino acid sequence of SEQ ID NO:646. In one aspect, provided herein is an antibody that binds PSMA, comprising a VH having an amino acid sequence having at least 95% identity to an amino acid sequence of SEQ ID NO:643. In one aspect, provided herein is an antibody that binds PSMA, comprising a VL having an amino acid sequence having at least 95% identity to an amino acid sequence of SEQ ID NO:508. In one aspect, provided herein is an antibody that binds PSMA, comprising a VH having an amino acid sequence having at least 95% identity to an amino acid sequence of SEQ ID NO:643, and a VL having an amino acid sequence having at least 95% identity to an amino acid sequence of SEQ ID NO:508. In one aspect, provided herein is an antibody that binds PSMA, comprising a heavy chain having an amino acid sequence having at least 95% identity to an amino acid sequence of SEQ ID NO:645. In one aspect, provided herein is an antibody that binds PSMA, comprising a light chain having an amino acid sequence having at least 95% identity to an amino acid sequence of SEQ ID NO:646. In one aspect, provided herein is an antibody that binds PSMA, comprising a heavy chain having an amino acid sequence having at least 95% identity to an amino acid sequence of SEQ ID NO:645, and a light chain having an amino acid sequence having at least 95% identity to an amino acid sequence of SEQ ID NO:646.

In one aspect, provided herein is an antibody that binds PSMA, comprising: (i) a VH comprising a VH CDR1, a VH CDR2, and a VH CDR3 having an amino acid sequence of SEQ ID NOs:647, 648, and 649, respectively, and (ii) a VL comprising a VL CDR1, VL CDR2, and VL CDR3 having an amino acid sequence of SEQ ID NOs:650, 549, and 652, respectively. In one aspect, provided herein is an antibody that binds PSMA, comprising: (i) a VH comprising a VH CDR1, a VH CDR2, and a VH CDR3 having an amino acid sequence of SEQ ID NOs:653, 518, and 655, respectively, and (ii) a VL comprising a VL CDR1, VL CDR2, and VL CDR3 having an amino acid sequence of SEQ ID NOs:656, 555, and 658, respectively. In one aspect, provided herein is an antibody that binds PSMA, comprising: (i) a VH comprising a VH CDR1, a VH CDR2, and a VH CDR3 having an amino acid sequence of SEQ ID NOs:659, 660, and 649, respectively, and (ii) a VL comprising a VL CDR1, VL CDR2, and VL CDR3 having an amino acid sequence of SEQ ID NOs:650, 549, and 652, respectively. In one aspect, provided herein is an antibody that binds PSMA, comprising: (i) a VH comprising a VH CDR1, a VH CDR2, and a VH CDR3 having an amino acid sequence of SEQ ID NOs:665, 666, and 667, respectively, and (ii) a VL comprising a VL CDR1, VL CDR2, and VL CDR3 having an amino acid sequence of SEQ ID NOs:566, 567, and 670, respectively. In one aspect, provided herein is an antibody that binds PSMA, comprising: (i) a VH comprising a VH CDR1, a VH CDR2, and a VH CDR3 having an amino acid sequence of SEQ ID NOs:671, 672, and 673, respectively, and (ii) a VL comprising a VL CDR1, VL CDR2, and VL CDR3 having an amino acid sequence of SEQ ID NOs:674, 555, and 652, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:677; and (ii) a VL comprising a VL CDR1, a VL CDR2, and a VL CDR3 having an amino acid sequence of a VL CDR1, a VL CDR2, and a VL CDR3, respectively, of SEQ ID NO:678. In one aspect, provided herein is an antibody that binds PSMA, comprising a VH having an amino acid sequence of SEQ ID NO:677. In one aspect, provided herein is an antibody that binds PSMA, comprising a VL having an amino acid sequence of SEQ ID NO:678. In one aspect, provided herein is an antibody that binds PSMA, comprising a VH having an amino acid sequence of SEQ ID NO:677, and a VL having an amino acid sequence of SEQ ID NO:678. In one aspect, provided herein is an antibody that binds PSMA, comprising a heavy chain having an amino acid sequence of SEQ ID NO:679. In one aspect, provided herein is an antibody that binds PSMA, comprising a light chain having an amino acid sequence of SEQ ID NO:680. In one aspect, provided herein is an antibody that binds PSMA, comprising a heavy chain having an amino acid sequence of SEQ ID NO:679, and a light chain having an amino acid sequence of SEQ ID NO:680. In one aspect, provided herein is an antibody that binds PSMA, comprising a VH having an amino acid sequence having at least 95% identity to an amino acid sequence of SEQ ID NO:677. In one aspect, provided herein is an antibody that binds PSMA, comprising a VL having an amino acid sequence having at least 95% identity to an amino acid sequence of SEQ ID NO:678. In one aspect, provided herein is an antibody that binds PSMA, comprising a VH having an amino acid sequence having at least 95% identity to an amino acid sequence of SEQ ID NO:677, and a VL having an amino acid sequence having at least 95% identity to an amino acid sequence of SEQ ID NO:678. In one aspect, provided herein is an antibody that binds PSMA, comprising a heavy chain having an amino acid sequence having at least 95% identity to an amino acid sequence of SEQ ID NO:679. In one aspect, provided herein is an antibody that binds PSMA, comprising a light chain having an amino acid sequence having at least 95% identity to an amino acid sequence of SEQ ID NO:680. In one aspect, provided herein is an antibody that binds PSMA, comprising a heavy chain having an amino acid sequence having at least 95% identity to an amino acid sequence of SEQ ID NO:679, and a light chain having an amino acid sequence having at least 95% identity to an amino acid sequence of SEQ ID NO:680.

In one aspect, provided herein is an antibody that binds PSMA, comprising: (i) a VH comprising a VH CDR1, a VH CDR2, and a VH CDR3 having an amino acid sequence of SEQ ID NOs:443, 682, and 445, respectively, and (ii) a VL comprising a VL CDR1, VL CDR2, and VL CDR3 having an amino acid sequence of SEQ ID NOs:446, 447, and 448, respectively. In one aspect, provided herein is an antibody that binds PSMA, comprising: (i) a VH comprising a VH CDR1, a VH CDR2, and a VH CDR3 having an amino acid sequence of SEQ ID NOs:143, 688, and 451, respectively, and (ii) a VL comprising a VL CDR1, VL CDR2, and VL CDR3 having an amino acid sequence of SEQ ID NOs:452, 453, and 454, respectively. In one aspect, provided herein is an antibody that binds PSMA, comprising: (i) a VH comprising a VH CDR1, a VH CDR2, and a VH CDR3 having an amino acid sequence of SEQ ID NOs:455, 694, and 445, respectively, and (ii) a VL comprising a VL CDR1, VL CDR2, and VL CDR3 having an amino acid sequence of SEQ ID NOs:446, 447, and 448, respectively. In one aspect, provided herein is an antibody that binds PSMA, comprising: (i) a VH comprising a VH CDR1, a VH CDR2, and a VH CDR3 having an amino acid sequence of SEQ ID NOs:461, 700, and 463, respectively, and (ii) a VL comprising a VL CDR1, VL CDR2, and VL CDR3 having an amino acid sequence of SEQ ID NOs:464, 465, and 466, respectively. In one aspect, provided herein is an antibody that binds PSMA, comprising: (i) a VH comprising a VH CDR1, a VH CDR2, and a VH CDR3 having an amino acid sequence of SEQ ID NOs:467, 706, and 469, respectively, and (ii) a VL comprising a VL CDR1, VL CDR2, and VL CDR3 having an amino acid sequence of SEQ ID NOs:470, 453, and 448, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:711; and (ii) a VL comprising a VL CDR1, a VL CDR2, and a VL CDR3 having an amino acid sequence of a VL CDR1, a VL CDR2, and a VL CDR3, respectively, of SEQ ID NO:474. In one aspect, provided herein is an antibody that binds PSMA, comprising a VH having an amino acid sequence of SEQ ID NO:711. In one aspect, provided herein is an antibody that binds PSMA, comprising a VL having an amino acid sequence of SEQ ID NO:474. In one aspect, provided herein is an antibody that binds PSMA, comprising a VH having an amino acid sequence of SEQ ID NO:711, and a VL having an amino acid sequence of SEQ ID NO:474. In one aspect, provided herein is an antibody that binds PSMA, comprising a heavy chain having an amino acid sequence of SEQ ID NO:713. In one aspect, provided herein is an antibody that binds PSMA, comprising a light chain having an amino acid sequence of SEQ ID NO:714. In one aspect, provided herein is an antibody that binds PSMA, comprising a heavy chain having an amino acid sequence of SEQ ID NO:713, and a light chain having an amino acid sequence of SEQ ID NO:714. In one aspect, provided herein is an antibody that binds PSMA, comprising a VH having an amino acid sequence having at least 95% identity to an amino acid sequence of SEQ ID NO:711. In one aspect, provided herein is an antibody that binds PSMA, comprising a VL having an amino acid sequence having at least 95% identity to an amino acid sequence of SEQ ID NO:474. In one aspect, provided herein is an antibody that binds PSMA, comprising a VH having an amino acid sequence having at least 95% identity to an amino acid sequence of SEQ ID NO:711, and a VL having an amino acid sequence having at least 95% identity to an amino acid sequence of SEQ ID NO:474. In one aspect, provided herein is an antibody that binds PSMA, comprising a heavy chain having an amino acid sequence having at least 95% identity to an amino acid sequence of SEQ ID NO:713. In one aspect, provided herein is an antibody that binds PSMA, comprising a light chain having an amino acid sequence having at least 95% identity to an amino acid sequence of SEQ ID NO:714. In one aspect, provided herein is an antibody that binds PSMA, comprising a heavy chain having an amino acid sequence having at least 95% identity to an amino acid sequence of SEQ ID NO:713, and a light chain having an amino acid sequence having at least 95% identity to an amino acid sequence of SEQ ID NO:714.

In one aspect, provided herein is an antibody that binds PSMA, comprising: (i) a VH comprising a VH CDR1, a VH CDR2, and a VH CDR3 having an amino acid sequence of SEQ ID NOs:715, 716, and 717, respectively, and (ii) a VL comprising a VL CDR1, VL CDR2, and VL CDR3 having an amino acid sequence of SEQ ID NOs:72, 719, and 720, respectively. In one aspect, provided herein is an antibody that binds PSMA, comprising: (i) a VH comprising a VH CDR1, a VH CDR2, and a VH CDR3 having an amino acid sequence of SEQ ID NOs:721, 722, and 723, respectively, and (ii) a VL comprising a VL CDR1, VL CDR2, and VL CDR3 having an amino acid sequence of SEQ ID NOs:78, 725, and 726, respectively. In one aspect, provided herein is an antibody that binds PSMA, comprising: (i) a VH comprising a VH CDR1, a VH CDR2, and a VH CDR3 having an amino acid sequence of SEQ ID NOs:727, 728, and 717, respectively, and (ii) a VL comprising a VL CDR1, VL CDR2, and VL CDR3 having an amino acid sequence of SEQ ID NOs:72, 719, and 720, respectively. In one aspect, provided herein is an antibody that binds PSMA, comprising: (i) a VH comprising a VH CDR1, a VH CDR2, and a VH CDR3 having an amino acid sequence of SEQ ID NOs:733, 734, and 735, respectively, and (ii) a VL comprising a VL CDR1, VL CDR2, and VL CDR3 having an amino acid sequence of SEQ ID NOs:736, 737, and 738, respectively. In one aspect, provided herein is an antibody that binds PSMA, comprising: (i) a VH comprising a VH CDR1, a VH CDR2, and a VH CDR3 having an amino acid sequence of SEQ ID NOs:739, 740, and 741, respectively, and (ii) a VL comprising a VL CDR1, VL CDR2, and VL CDR3 having an amino acid sequence of SEQ ID NOs:96, 725, and 720, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:745; and (ii) a VL comprising a VL CDR1, a VL CDR2, and a VL CDR3 having an amino acid sequence of a VL CDR1, a VL CDR2, and a VL CDR3, respectively, of SEQ ID NO:746. In one aspect, provided herein is an antibody that binds PSMA, comprising a VH having an amino acid sequence of SEQ ID NO:745. In one aspect, provided herein is an antibody that binds PSMA, comprising a VL having an amino acid sequence of SEQ ID NO:746. In one aspect, provided herein is an antibody that binds PSMA, comprising a VH having an amino acid sequence of SEQ ID NO:745, and a VL having an amino acid sequence of SEQ ID NO:746. In one aspect, provided herein is an antibody that binds PSMA, comprising a heavy chain having an amino acid sequence of SEQ ID NO:747. In one aspect, provided herein is an antibody that binds PSMA, comprising a light chain having an amino acid sequence of SEQ ID NO:748. In one aspect, provided herein is an antibody that binds PSMA, comprising a heavy chain having an amino acid sequence of SEQ ID NO:747, and a light chain having an amino acid sequence of SEQ ID NO:748. In one aspect, provided herein is an antibody that binds PSMA, comprising a VH having an amino acid sequence having at least 95% identity to an amino acid sequence of SEQ ID NO:745. In one aspect, provided herein is an antibody that binds PSMA, comprising a VL having an amino acid sequence having at least 95% identity to an amino acid sequence of SEQ ID NO:746. In one aspect, provided herein is an antibody that binds PSMA, comprising a VH having an amino acid sequence having at least 95% identity to an amino acid sequence of SEQ ID NO:745, and a VL having an amino acid sequence having at least 95% identity to an amino acid sequence of SEQ ID NO:746. In one aspect, provided herein is an antibody that binds PSMA, comprising a heavy chain having an amino acid sequence having at least 95% identity to an amino acid sequence of SEQ ID NO:747. In one aspect, provided herein is an antibody that binds PSMA, comprising a light chain having an amino acid sequence having at least 95% identity to an amino acid sequence of SEQ ID NO:748. In one aspect, provided herein is an antibody that binds PSMA, comprising a heavy chain having an amino acid sequence having at least 95% identity to an amino acid sequence of SEQ ID NO:747, and a light chain having an amino acid sequence having at least 95% identity to an amino acid sequence of SEQ ID NO:748.

In one aspect, provided herein is an antibody that binds PSMA, comprising: (i) a VH comprising a VH CDR1, a VH CDR2, and a VH CDR3 having an amino acid sequence of SEQ ID NOs:647, 750, and 751, respectively, and (ii) a VL comprising a VL CDR1, VL CDR2, and VL CDR3 having an amino acid sequence of SEQ ID NOs:752, 481, and 754, respectively. In one aspect, provided herein is an antibody that binds PSMA, comprising: (i) a VH comprising a VH CDR1, a VH CDR2, and a VH CDR3 having an amino acid sequence of SEQ ID NOs:653, 518, and 757, respectively, and (ii) a VL comprising a VL CDR1, VL CDR2, and VL CDR3 having an amino acid sequence of SEQ ID NOs:758, 487, and 760, respectively. In one aspect, provided herein is an antibody that binds PSMA, comprising: (i) a VH comprising a VH CDR1, a VH CDR2, and a VH CDR3 having an amino acid sequence of SEQ ID NOs:659, 762, and 751, respectively, and (ii) a VL comprising a VL CDR1, VL CDR2, and VL CDR3 having an amino acid sequence of SEQ ID NOs:752, 481, and 754, respectively. In one aspect, provided herein is an antibody that binds PSMA, comprising: (i) a VH comprising a VH CDR1, a VH CDR2, and a VH CDR3 having an amino acid sequence of SEQ ID NOs:665, 768, and 769, respectively, and (ii) a VL comprising a VL CDR1, VL CDR2, and VL CDR3 having an amino acid sequence of SEQ ID NOs:498, 499, and 772, respectively. In one aspect, provided herein is an antibody that binds PSMA, comprising: (i) a VH comprising a VH CDR1, a VH CDR2, and a VH CDR3 having an amino acid sequence of SEQ ID NOs:671, 536, and 775, respectively, and (ii) a VL comprising a VL CDR1, VL CDR2, and VL CDR3 having an amino acid sequence of SEQ ID NOs:504, 487, and 754, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:779; and (ii) a VL comprising a VL CDR1, a VL CDR2, and a VL CDR3 having an amino acid sequence of a VL CDR1, a VL CDR2, and a VL CDR3, respectively, of SEQ ID NO:780. In one aspect, provided herein is an antibody that binds PSMA, comprising a VH having an amino acid sequence of SEQ ID NO:779. In one aspect, provided herein is an antibody that binds PSMA, comprising a VL having an amino acid sequence of SEQ ID NO:780. In one aspect, provided herein is an antibody that binds PSMA, comprising a VH having an amino acid sequence of SEQ ID NO:779, and a VL having an amino acid sequence of SEQ ID NO:780. In one aspect, provided herein is an antibody that binds PSMA, comprising a heavy chain having an amino acid sequence of SEQ ID NO:781. In one aspect, provided herein is an antibody that binds PSMA, comprising a light chain having an amino acid sequence of SEQ ID NO:782. In one aspect, provided herein is an antibody that binds PSMA, comprising a heavy chain having an amino acid sequence of SEQ ID NO:781, and a light chain having an amino acid sequence of SEQ ID NO:782. In one aspect, provided herein is an antibody that binds PSMA, comprising a VH having an amino acid sequence having at least 95% identity to an amino acid sequence of SEQ ID NO:779. In one aspect, provided herein is an antibody that binds PSMA, comprising a VL having an amino acid sequence having at least 95% identity to an amino acid sequence of SEQ ID NO:780. In one aspect, provided herein is an antibody that binds PSMA, comprising a VH having an amino acid sequence having at least 95% identity to an amino acid sequence of SEQ ID NO:779, and a VL having an amino acid sequence having at least 95% identity to an amino acid sequence of SEQ ID NO:780. In one aspect, provided herein is an antibody that binds PSMA, comprising a heavy chain having an amino acid sequence having at least 95% identity to an amino acid sequence of SEQ ID NO:781. In one aspect, provided herein is an antibody that binds PSMA, comprising a light chain having an amino acid sequence having at least 95% identity to an amino acid sequence of SEQ ID NO:782. In one aspect, provided herein is an antibody that binds PSMA, comprising a heavy chain having an amino acid sequence having at least 95% identity to an amino acid sequence of SEQ ID NO:781, and a light chain having an amino acid sequence having at least 95% identity to an amino acid sequence of SEQ ID NO:782.

In one aspect, provided herein is an antibody that binds PSMA, comprising: (i) a VH comprising a VH CDR1, a VH CDR2, and a VH CDR3 having an amino acid sequence of SEQ ID NOs:783, 784, and 785, respectively, and (ii) a VL comprising a VL CDR1, VL CDR2, and VL CDR3 having an amino acid sequence of SEQ ID NOs:548, 549, and 788, respectively. In one aspect, provided herein is an antibody that binds PSMA, comprising: (i) a VH comprising a VH CDR1, a VH CDR2, and a VH CDR3 having an amino acid sequence of SEQ ID NOs:653, 518, and 791, respectively, and (ii) a VL comprising a VL CDR1, VL CDR2, and VL CDR3 having an amino acid sequence of SEQ ID NOs:554, 555, and 794, respectively. In one aspect, provided herein is an antibody that binds PSMA, comprising: (i) a VH comprising a VH CDR1, a VH CDR2, and a VH CDR3 having an amino acid sequence of SEQ ID NOs:795, 796, and 785, respectively, and (ii) a VL comprising a VL CDR1, VL CDR2, and VL CDR3 having an amino acid sequence of SEQ ID NOs:548, 549, and 788, respectively. In one aspect, provided herein is an antibody that binds PSMA, comprising: (i) a VH comprising a VH CDR1, a VH CDR2, and a VH CDR3 having an amino acid sequence of SEQ ID NOs:801, 802, and 803, respectively, and (ii) a VL comprising a VL CDR1, VL CDR2, and VL CDR3 having an amino acid sequence of SEQ ID NOs:566, 567, and 806, respectively. In one aspect, provided herein is an antibody that binds PSMA, comprising: (i) a VH comprising a VH CDR1, a VH CDR2, and a VH CDR3 having an amino acid sequence of SEQ ID NOs:671, 536, and 809, respectively, and (ii) a VL comprising a VL CDR1, VL CDR2, and VL CDR3 having an amino acid sequence of SEQ ID NOs:572, 555, and 788, respectively. In one aspect, provided herein is an antibody that binds PSMA, comprising: (i) a VH comprising a VH CDR1, a VH CDR2, and a VH CDR3 having an amino acid sequence of a VH CDR1, a VH CDR2, and a VH CDR3, respectively, of SEQ ID NO:813; and (ii) a VL comprising a VL CDR1, a VL CDR2, and a VL CDR3 having an amino acid sequence of a VL CDR1, a VL CDR2, and a VL CDR3, respectively, of SEQ ID NO:814. In one aspect, provided herein is an antibody that binds PSMA, comprising a VH having an amino acid sequence of SEQ ID NO:813. In one aspect, provided herein is an antibody that binds PSMA, comprising a VL having an amino acid sequence of SEQ ID NO:814. In one aspect, provided herein is an antibody that binds PSMA, comprising a VH having an amino acid sequence of SEQ ID NO:813, and a VL having an amino acid sequence of SEQ ID NO:814. In one aspect, provided herein is an antibody that binds PSMA, comprising a heavy chain having an amino acid sequence of SEQ ID NO:815. In one aspect, provided herein is an antibody that binds PSMA, comprising a light chain having an amino acid sequence of SEQ ID NO:816. In one aspect, provided herein is an antibody that binds PSMA, comprising a heavy chain having an amino acid sequence of SEQ ID NO:815, and a light chain having an amino acid sequence of SEQ ID NO:816. In one aspect, provided herein is an antibody that binds PSMA, comprising a VH having an amino acid sequence having at least 95% identity to an amino acid sequence of SEQ ID NO:813. In one aspect, provided herein is an antibody that binds PSMA, comprising a VL having an amino acid sequence having at least 95% identity to an amino acid sequence of SEQ ID NO:814. In one aspect, provided herein is an antibody that binds PSMA, comprising a VH having an amino acid sequence having at least 95% identity to an amino acid sequence of SEQ ID NO:813, and a VL having an amino acid sequence having at least 95% identity to an amino acid sequence of SEQ ID NO:814. In one aspect, provided herein is an antibody that binds PSMA, comprising a heavy chain having an amino acid sequence having at least 95% identity to an amino acid sequence of SEQ ID NO:815. In one aspect, provided herein is an antibody that binds PSMA, comprising a light chain having an amino acid sequence having at least 95% identity to an amino acid sequence of SEQ ID NO:816. In one aspect, provided herein is an antibody that binds PSMA, comprising a heavy chain having an amino acid sequence having at least 95% identity to an amino acid sequence of SEQ ID NO:815, and a light chain having an amino acid sequence having at least 95% identity to an amino acid sequence of SEQ ID NO:816.

In some embodiments, the isolated antibody comprises a VH having an amino acid sequence at least 80% identical to the amino acid sequence of SEQ ID NO:31. In some embodiments, the isolated antibody comprises a VH having an amino acid sequence at least 80% identical to the amino acid sequence of SEQ ID NO:99. In some embodiments, the isolated antibody comprises a VH having an amino acid sequence at least 80% identical to the amino acid sequence of SEQ ID NO:167. In some embodiments, the isolated antibody comprises a VH having an amino acid sequence at least 80% identical to the amino acid sequence of SEQ ID NO:235. In some embodiments, the isolated antibody comprises a VH having an amino acid sequence at least 80% identical to the amino acid sequence of SEQ ID NO:303. In some embodiments, the isolated antibody comprises a VH having an amino acid sequence at least 80% identical to the amino acid sequence of SEQ ID NO:371. In some embodiments, the isolated antibody comprises a VH having an amino acid sequence at least 80% identical to the amino acid sequence of SEQ ID NO:405. In some embodiments, the isolated antibody comprises a VH having an amino acid sequence at least 80% identical to the amino acid sequence of SEQ ID NO:439. In some embodiments, the isolated antibody comprises a VH having an amino acid sequence at least 80% identical to the amino acid sequence of SEQ ID NO:473. In some embodiments, the isolated antibody comprises a VH having an amino acid sequence at least 80% identical to the amino acid sequence of SEQ ID NO:507. In some embodiments, the isolated antibody comprises a VH having an amino acid sequence at least 80% identical to the amino acid sequence of SEQ ID NO:541. In some embodiments, the isolated antibody comprises a VH having an amino acid sequence at least 80% identical to the amino acid sequence of SEQ ID NO:575. In some embodiments, the isolated antibody comprises a VH having an amino acid sequence at least 80% identical to the amino acid sequence of SEQ ID NO:643. In some embodiments, the isolated antibody comprises a VH having an amino acid sequence at least 80% identical to the amino acid sequence of SEQ ID NO:677. In some embodiments, the isolated antibody comprises a VH having an amino acid sequence at least 80% identical to the amino acid sequence of SEQ ID NO:711. In some embodiments, the isolated antibody comprises a VH having an amino acid sequence at least 80% identical to the amino acid sequence of SEQ ID NO:779. In some embodiments, the isolated antibody comprises a VH having an amino acid sequence at least 80% identical to the amino acid sequence of SEQ ID NO:813.

In some embodiments, the isolated antibody comprises a VL having an amino acid sequence at least 80% identical to the amino acid sequence of SEQ ID NO:32. In some embodiments, the isolated antibody comprises a VL having an amino acid sequence at least 80% identical to the amino acid sequence of SEQ ID NO:66. In some embodiments, the isolated antibody comprises a VL having an amino acid sequence at least 80% identical to the amino acid sequence of SEQ ID NO:100. In some embodiments, the isolated antibody comprises a VL having an amino acid sequence at least 80% identical to the amino acid sequence of SEQ ID NO:134. In some embodiments, the isolated antibody comprises a VL having an amino acid sequence at least 80% identical to the amino acid sequence of SEQ ID NO:236. In some embodiments, the isolated antibody comprises a VL having an amino acid sequence at least 80% identical to the amino acid sequence of SEQ ID NO:270. In some embodiments, the isolated antibody comprises a VL having an amino acid sequence at least 80% identical to the amino acid sequence of SEQ ID NO:372. In some embodiments, the isolated antibody comprises a VL having an amino acid sequence at least 80% identical to the amino acid sequence of SEQ ID NO:406. In some embodiments, the isolated antibody comprises a VL having an amino acid sequence at least 80% identical to the amino acid sequence of SEQ ID NO:474. In some embodiments, the isolated antibody comprises a VL having an amino acid sequence at least 80% identical to the amino acid sequence of SEQ ID NO:508. In some embodiments, the isolated antibody comprises a VL having an amino acid sequence at least 80% identical to the amino acid sequence of SEQ ID NO:542. In some embodiments, the isolated antibody comprises a VL having an amino acid sequence at least 80% identical to the amino acid sequence of SEQ ID NO:576. In some embodiments, the isolated antibody comprises a VL having an amino acid sequence at least 80% identical to the amino acid sequence of SEQ ID NO:678. In some embodiments, the isolated antibody comprises a VL having an amino acid sequence at least 80% identical to the amino acid sequence of SEQ ID NO:746. In some embodiments, the isolated antibody comprises a VL having an amino acid sequence at least 80% identical to the amino acid sequence of SEQ ID NO:780. In some embodiments, the isolated antibody comprises a VL having an amino acid sequence at least 80% identical to the amino acid sequence of SEQ ID NO:814.

In some embodiments, the isolated antibody comprises a heavy chain (HC) having an amino acid sequence at least 80% identical to the amino acid sequence of SEQ ID NO:33. In some embodiments, the isolated antibody comprises a HC having an amino acid sequence at least 80% identical to the amino acid sequence of SEQ ID NO:101. In some embodiments, the isolated antibody comprises a HC having an amino acid sequence at least 80% identical to the amino acid sequence of SEQ ID NO:169. In some embodiments, the isolated antibody comprises a HC having an amino acid sequence at least 80% identical to the amino acid sequence of SEQ ID NO:237. In some embodiments, the isolated antibody comprises a HC having an amino acid sequence at least 80% identical to the amino acid sequence of SEQ ID NO:305. In some embodiments, the isolated antibody comprises a HC having an amino acid sequence at least 80% identical to the amino acid sequence of SEQ ID NO:373. In some embodiments, the isolated antibody comprises a HC having an amino acid sequence at least 80% identical to the amino acid sequence of SEQ ID NO:407. In some embodiments, the isolated antibody comprises a HC having an amino acid sequence at least 80% identical to the amino acid sequence of SEQ ID NO:441.

In some embodiments, the isolated antibody comprises a light chain (LC) having an amino acid sequence at least 80% identical to the amino acid sequence of SEQ ID NO:34. In some embodiments, the isolated antibody comprises a LC having an amino acid sequence at least 80% identical to the amino acid sequence of SEQ ID NO:68. In some embodiments, the isolated antibody comprises a LC having an amino acid sequence at least 80% identical to the amino acid sequence of SEQ ID NO:102. In some embodiments, the isolated antibody comprises a LC having an amino acid sequence at least 80% identical to the amino acid sequence of SEQ ID NO:136. In some embodiments, the isolated antibody comprises a LC having an amino acid sequence at least 80% identical to the amino acid sequence of SEQ ID NO:238. In some embodiments, the isolated antibody comprises a LC having an amino acid sequence at least 80% identical to the amino acid sequence of SEQ ID NO:272. In some embodiments, the isolated antibody comprises a LC having an amino acid sequence at least 80% identical to the amino acid sequence of SEQ ID NO:374. In some embodiments, the isolated antibody comprises a LC having an amino acid sequence at least 80% identical to the amino acid sequence of SEQ ID NO:408.

In some embodiments, provided herein is an isolated antibody that binds PSMA comprising (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In some embodiments, provided herein is an isolated antibody that binds PSMA comprising (i) a VH comprising a VH CDR1, a VH CDR2, and a VH CDR3 having an amino acid sequence of SEQ ID NOs:205, 206, and 411, respectively, and (ii) a VL comprising a VL CDR1, VL CDR2, and VL CDR3 having an amino acid sequence of SEQ ID NOs:242, 209, and 244, respectively. In some embodiments, provided herein is an isolated antibody that binds PSMA comprising (i) a VH having an amino acid sequence of SEQ ID NO:439; and (ii) a VL having an amino acid sequence of SEQ ID NO:270. In some embodiments, provided herein is an isolated antibody that binds PSMA comprising (i) a HC having an amino acid sequence of SEQ ID NO:441; and (ii) a LC having an amino acid sequence of SEQ ID NO:272.

In another aspect, provided herein is an antibody that competes for binding to PSMA with any of the PSMA antibodies described herein. In another aspect, provided herein is an antibody that binds to the same epitope as any of the PSMA antibodies described herein. In another aspect, provided is a PSMA antibody that binds an epitope on PSMA that overlaps with the epitope on PSMA bound by a PSMA antibody described herein.

In one aspect, provided is an antibody that competes for binding to PSMA with a PSMA reference antibody. In another aspect, provided is a PSMA antibody that binds to the same PSMA epitope as a PSMA reference antibody. In another aspect, provided is a PSMA antibody that binds an epitope on PSMA that overlaps with the epitope on PSMA bound by a PSMA reference antibody.

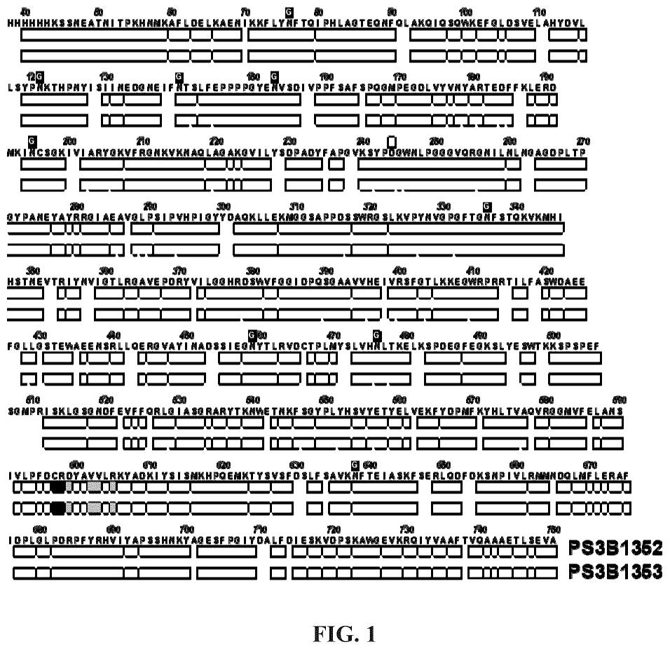

In some embodiments, the epitope is as provided in FIG. 1. In some embodiments, the antibody binds to one or more of amino acid residues 597, 598, 599, 600, 601, 602, 603, 604, 605, 606, 607, 608, 609, 610, 611, or 612 of the amino acid sequences of PSMA as shown in FIG. 1.

In some embodiments, the antibody binds to amino acid residue 597 of the amino acid sequence of PSMA as provided in FIG. 1. In some embodiments, the antibody binds to amino acid residue 598 of the amino acid sequence of PSMA as provided in FIG. 1. In some embodiments, the antibody binds to amino acid residue 599 of the amino acid sequence of PSMA as provided in FIG. 1. In some embodiments, the antibody binds to amino acid residue 600 of the amino acid sequence of PSMA as provided in FIG. 1. In some embodiments, the antibody binds to amino acid residue 601 of the amino acid sequence of PSMA as provided in FIG. 1. In some embodiments, the antibody binds to amino acid residue 602 of the amino acid sequence of PSMA as provided in FIG. 1. In some embodiments, the antibody binds to amino acid residue 603 of the amino acid sequence of PSMA as provided in FIG. 1. In some embodiments, the antibody binds to amino acid residue 604 of the amino acid sequence of PSMA as provided in FIG. 1. In some embodiments, the antibody binds to amino acid residue 605 of the amino acid sequence of PSMA as provided in FIG. 1. In some embodiments, the antibody binds to amino acid residue 606 of the amino acid sequence of PSMA as provided in FIG. 1. In some embodiments, the antibody binds to amino acid residue 607 of the amino acid sequence of PSMA as provided in FIG. 1. In some embodiments, the antibody binds to amino acid residue 608 of the amino acid sequence of PSMA as provided in FIG. 1. In some embodiments, the antibody binds to amino acid residue 609 of the amino acid sequence of PSMA as provided in FIG. 1. In some embodiments, the antibody binds to amino acid residue 610 of the amino acid sequence of PSMA as provided in FIG. 1. In some embodiments, the antibody binds to amino acid residue 611 of the amino acid sequence of PSMA as provided in FIG. 1. In some embodiments, the antibody binds to amino acid residue 612 of the amino acid sequence of PSMA as provided in FIG. 1. In some embodiments, the antibody binds to one or more residues selected from 597-598 (CR) of PSMA as shown in FIG. 1. In some embodiments, the antibody binds to one or more residues selected from 593-594 (LP), 595 (F), 596 (D), 600 (Y), 601 (A), 604 (L), 606-607 (KY), 607-609 (YAD), and 610-612 (MY) of PSMA as shown in FIG. 1.

In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the PSMA reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814.

In certain embodiments, provided are multispecific antibodies that specifically bind to PSMA. According to certain embodiments, a bispecific antibody can be used to engage two different therapeutic targets or perform two distinct functions. Such antibodies can be used for example to recruit an immune effector cell, e.g., T- or NK-cell, towards a particular target cell. Various antibody-fragment based molecules are known and under investigation, for example for cancer therapy. A multispecific PSMA antibody provided herein can be a trispecific antibody for dual targeting of tumor cells—these are trifunctional structures that can be designed to target two different targets/epitopes on the tumor cell and with the third functionality bind with high affinity to either T cells or NK-cells. Trispecific antibodies targeting two distinct tumor epitopes and engaging T- or NK-cells lyse the tumor cells that express both targets. Such molecules can be generated by antibody formats known in the art and are fully described. (WO20151842071, WO2015158636, WO2010136172, WO2013174873). In a trispecific antibody embodiment, the multispecific antibody is specific for PSMA and a second distinct antigen on the same or another tumor cell and additionally specific for an effector cell, in particular a T cell or an NK cell.

Also provided are a PSMA X “effector antigen” bispecific antibody. In one embodiment, the effector antigen of the PSMA X “effector antigen” bispecific antibody is CD3. Certain multispecific PSMAxCD3 antibodies provided can be used in preclinical animal testing, as well as clinical studies and even in therapy in human. This is due to the identification of the PSMAxCD3 bispecific antibody, which, in addition to binding to human PSMA and human CD3, respectively, also binds to the homologs of antigens of chimpanzee and macaques. For example, PSMAxCD3 multispecific antibodies provided herein can be used as a therapeutic agent against various diseases, including, but not limited, to cancer. In view of the above, the need to construct a surrogate target PSMAxCD3 multispecific antibody for testing in a phylogenetically distant (from humans) species disappears. As a result, the identical molecule can be used in animal preclinical testing as is intended to be administered to humans in clinical testing as well as following market approval and therapeutic drug administration. The ability to use the same molecule for preclinical animal testing as in later administration to humans virtually eliminates, or at least greatly reduces, the danger that the data obtained in preclinical animal testing have limited applicability to the human case. In short, obtaining preclinical safety data in animals using the same molecule as will actually be administered to humans does much to ensure the applicability of the data to a human-relevant scenario. In contrast, in conventional approaches using surrogate molecules, said surrogate molecules have to be molecularly adapted to the animal test system used for preclinical safety assessment. Thus, the molecule to be used in human therapy in fact differs in sequence and also likely in structure from the surrogate molecule used in preclinical testing in pharmacokinetic parameters and/or biological activity, with the consequence that data obtained in preclinical animal testing have limited applicability/transferability to the human case. The use of surrogate molecules requires the construction, production, purification and characterization of a completely new construct. This leads to additional development costs and time necessary to obtain that molecule. In sum, surrogates have to be developed separately in addition to the actual drug to be used in human therapy, so that two lines of development for two molecules have to be carried out. Therefore, a major advantage of the PSMAxCD3 multispecific antibodies provided herein exhibiting cross-species specificity described herein is that the identical molecule can be used for therapeutic agents in humans and in preclinical animal testing.

Another major advantage of the antibodies and multispecific antibodies provided herein is the applicability for preclinical testing in various primates. The behavior of a drug candidate in animals should ideally be indicative of the expected behavior of this drug candidate upon administration to humans. As a result, the data obtained from such preclinical testing should therefore generally have a highly predictive power for the human case. However, as learned from the recent Phase I clinical trial on TGN1412 (a CD28 monoclonal antibody), a drug candidate can act differently in a primate species than in humans: Whereas in preclinical testing of the antibody, no or only limited adverse effects have been observed in animal studies performed with cynomolgus monkeys, six human patients developed multiple organ failure upon administration of the antibody (Lancet 368 (2006), 2206-7). The results of these dramatic negative events suggest that it may not be sufficient to limit preclinical testing to only one (non-chimpanzee primate) species. The fact that certain antibodies and multispecific antibodies provided herein specifically bind PSMA of chimpanzee and cynomolgus monkey can help to overcome the problems faced in the case mentioned above. Accordingly, provided are means and methods for minimizing species differences in effects when drugs for human therapy are being developed and tested.

With the antibodies and multispecific antibodies provided herein it is also no longer necessary to adapt the test animal to the drug candidate intended for administration to humans, such as e.g., the creation of transgenic animals. The cross-species specificity of the PSMA antibody or multispecific antibody provided herein allows the antibody to be directly used for preclinical testing in non-chimpanzee primates without any genetic manipulation of the animals. As well known to those skilled in the art, approaches in which the test animal is adapted to the drug candidate always bear the risk that the results obtained in the preclinical safety testing are less representative and predictive for humans due to the modification of the animal. For example, in transgenic animals, the proteins encoded by the transgenes are often highly over-expressed. Thus, data obtained for the biological activity of an antibody against this protein antigen can be limited in their predictive value for humans in which the protein is expressed at much lower, more physiological levels.

A further advantage of the uses of certain antibodies provided herein exhibiting cross-species specificity is the fact that the use of chimpanzees, an endangered species, can be avoided for animal testing. Chimpanzees are the closest relatives to humans and were recently grouped into the family of hominids based on the genome sequencing data (Wildman et al., PNAS 100 (2003), 7181). Therefore, data obtained with chimpanzee is generally considered to be highly predictive for humans. However, due to their status as endangered species, the number of chimpanzees, which can be used for medical experiments, is highly restricted. As stated above, maintenance of chimpanzees for animal testing is therefore both costly and ethically problematic. The uses of the antibodies provided herein avoid both ethical objections and financial burden during preclinical testing without prejudicing the quality, i.e., applicability, of the animal testing data obtained. In light of this, the uses of the antibody or multispecific antibody specifically binding PSMA provide for a reasonable alternative for studies in chimpanzees.

A still further advantage of certain antibodies or multispecific antibodies provided herein that bind to PSMA is the ability of extracting multiple blood samples when using it as part of animal preclinical testing, for example in the course of pharmacokinetic animal studies. Multiple blood extractions can be much more readily obtained with a non-chimpanzee primate than with lower animals, e.g., a mouse. The extraction of multiple blood samples allows continuous testing of blood parameters for the determination of the biological effects induced by the antibody or multispecific antibody specifically binding PSMA provided herein. Furthermore, the extraction of multiple blood samples enables the researcher to evaluate the pharmacokinetic profile of the antibody or multispecific antibody specifically binding PSMA provided herein as defined herein. In addition, potential side effects, which can be induced by said antibody or multispecific antibody specifically binding PSMA provided herein reflected in blood parameters can be measured in different blood samples extracted during the course of the administration of said antibody. This can allow the determination of the potential toxicity profile of the antibodies or multispecific antibodies provided herein.

In some embodiments, the PSMA antibody is PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In some embodiments, the PSMA antibody is PSMB946 (PSMB895 with a C-terminal Lys (K) amino acid residue). In some embodiments, the PSMA antibody is PSMB947 (PSMB896 with a C-terminal Lys (K) amino acid residue). In some embodiments, the PSMA antibody is PSMB948 (PSMB897 with a C-terminal Lys (K) amino acid residue). In some embodiments, the PSMA antibody is PSMB949 (PSMB898 with a C-terminal Lys (K) amino acid residue). In certain embodiments, the PSMA antibody is PSMB889. In other embodiments, the PSMA antibody is PSMB890. In certain embodiments, the PSMA antibody is PSMB891. In certain embodiments, the PSMA antibody is PSMB892. In other embodiments, the PSMA antibody is PSMB893. In certain embodiments, the PSMA antibody is PSMB894. In other embodiments, the PSMA antibody is PSMB895. In certain embodiments, the PSMA antibody is PSMB896. In certain embodiments, the PSMA antibody is PSMB897. In other embodiments, the PSMA antibody is PSMB898. In certain embodiments, the PSMA antibody is PSMB899. In certain embodiments, the PSMA antibody is PSMHB49SC1133_011A11_1. In other embodiments, the PSMA antibody is PSMB896-G100A. In certain embodiments, the PSMA antibody is PSMA_P72_A10-HC-G54E. In certain embodiments, the PSMA antibody is PSMA_P72_D01-HC-D95E. In other embodiments, the PSMA antibody is PSMA_P72_F01. In other embodiments, the PSMA antibody is PSMA_P75_F01. In certain embodiments, the PSMA antibody is PSMA_P72_F07. In certain embodiments, the PSMA antibody is PSMA_P72_E07. In other embodiments, the PSMA antibody is PSMA_P72_D01. In certain embodiments, the PSMA antibody is PSMA_P72_C01. In other embodiments, the PSMA antibody is PSMA_P72_A10. In certain embodiments, the PSMA antibody is PSMA_P72_F02. In certain embodiments, the PSMA antibody is PSMA_P70_F02. In other embodiments, the PSMA antibody is PSMA_P72_G02. In other embodiments, the PSMA antibody is PSMA_P72_A11. Each of these antibodies is further described in the Examples below, including Tables 4-12.

In some embodiments, provided is a PSMA antibody comprising a VH of any of the PSMA antibodies provided in Tables 4-12 or 23-28. In some embodiments, provided is a PSMA antibody comprising a VL of any of the PSMA antibodies provided in Tables 4-12 or 23-28. In some embodiments, provided is a PSMA antibody comprising a VH and VL of any of the PSMA antibodies provided in Tables 4-12 or 23-28. In other embodiments, provided is a PSMA antibody comprising a VH CDR1, VH CDR3 and VH CDR3 of any of the PSMA antibodies provided in Tables 4-12 or 23-28. In other embodiments, provided is a PSMA antibody comprising a VL CDR1, VL CDR3 and VL CDR3 of any of the PSMA antibodies provided in Tables 4-12 or 23-28. In other embodiments, provided is a PSMA antibody comprising a VH CDR1, VH CDR3, VH CDR3, VL CDR1, VL CDR2, and VL CDR3 of any of the PSMA antibodies provided in Tables 4-12 or 23-28. In some embodiments, the VH CDR1, VH CDR2, VH CDR3, VL CDR1, VL CDR2, and VL CDR3 amino acid sequences of the PSMA antibody are according to the Kabat numbering system. In some embodiments, the VH CDR1, VH CDR2, VH CDR3, VL CDR1, VL CDR2, and VL CDR3 amino acid sequences of the PSMA antibody are according to the Chothia numbering system. In some embodiments, the VH CDR1, VH CDR2, VH CDR3, VL CDR1, VL CDR2, and VL CDR3 amino acid sequences of the PSMA antibody are according to the AbM numbering system. In some embodiments, the VH CDR1, VH CDR2, VH CDR3, VL CDR1, VL CDR2, and VL CDR3 amino acid sequences of the PSMA antibody are according to the Contact numbering system. In some embodiments, the VH CDR1, VH CDR2, VH CDR3, VL CDR1, VL CDR2, and VL CDR3 amino acid sequences of the PSMA antibody are according to the IMGT numbering system. In some embodiments, the VH CDR1, VH CDR2, VH CDR3, VL CDR1, VL CDR2, and VL CDR3 sequences of the PSMA antibody are according to a combination of the numbering systems provided herein.

In some embodiments, the PSMA antibody comprises the VH of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72 COL PSMA_P72_A10, PSMA_P72_F02, PSMA_P70_F02, PSMA_P72_G02, or PSMA_P72_A11. In some embodiments, the PSMA antibody comprises the VL of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72 COL PSMA_P72_A10, PSMA_P72_F02, PSMA_P70_F02, PSMA_P72_G02, or PSMA_P72_A11. In some embodiments, the PSMA antibody comprises the both the VH and the VL of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72 COL PSMA_P72_A10, PSMA_P72_F02, PSMA_P70_F02, PSMA_P72_G02, or PSMA_P72_A11. In a specific embodiment, the VH and VL are of a single PSMA antibody clone.

In some embodiments, the PSMA antibody is a multispecific PSMA antibody, wherein the first binding domain that binds PSMA comprises the VH of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72 COL PSMA_P72_A10, PSMA_P72_F02, PSMA_P70_F02, PSMA_P72_G02, or PSMA_P72_A11. In some embodiments, the PSMA antibody is a multispecific PSMA antibody, wherein the first binding domain that binds PSMA comprises the VL of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In some embodiments, the PSMA antibody is a multispecific PSMA antibody, wherein the first binding domain that binds PSMA comprises both the VH and VL of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In a specific embodiment, the VH and VL are of a single PSMA antibody clone. In certain embodiments, the multispecific PSMA antibody is a multispecific PSMAxCD3 antibody. In specific embodiments, the multispecific PSMAxCD3 antibody is a bispecific PSMAxCD3 antibody. In some embodiments, the second binding domain that binds CD3 comprises the VH of CD3B376. In some embodiments, the second binding domain that binds CD3 comprises the VL of CD3B376. In some embodiments, the second binding domain that binds CD3 comprises both the VH and VL of CD3B376. In some embodiments, the second binding domain that binds CD3 comprises the VH of CD3B450. In some embodiments, the second binding domain that binds CD3 comprises the VL of CD3B450. In some embodiments, the second binding domain that binds CD3 comprises the both the VH and VL of CD3B450. In some embodiments, the second binding domain that binds CD3 comprises the VH of CD3W245. In some embodiments, the second binding domain that binds CD3 comprises the VL of CD3W245. In some embodiments, the second binding domain that binds CD3 comprises both the VH and VL of CD3W245. In some embodiments, the second binding domain that binds CD3 comprises the VH of CD3B2030. In some embodiments, the second binding domain that binds CD3 comprises the VL of CD3B2030. In some embodiments, the second binding domain that binds CD3 comprises both the VH and VL of CD3B2030.

In some embodiments, the PSMA antibody comprises the VH CDR1-3 of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In some embodiments, the PSMA antibody comprises the VL CDR1-3 of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In some embodiments, the PSMA antibody comprises both the VH CDR1-3 and the VL CDR1-3 of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In a specific embodiment, the VH CDR1-3 and VL CDR1-3 are of a single PSMA antibody clone.

In some embodiments, the PSMA antibody is a multispecific PSMA antibody, wherein the first binding domain that binds PSMA comprises the VH CDR1-3 of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In some embodiments, the PSMA antibody is a multispecific PSMA antibody, wherein the first binding domain that binds PSMA comprises the VL CDR1-3 of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P72_E07, PSMA_P72_D01, PSMA_P72_C01, PSMA_P72_A10, PSMA_P72_F02, PSMA_P70_F02, PSMA_P72_G02, or PSMA_P72_A11. In some embodiments, the PSMA antibody is a multispecific PSMA antibody, wherein the first binding domain that binds PSMA comprises both the VH CDR1-3 and VL CDR1-3 of PSMB889, PSMB890, PSMB891, PSMB892, PSMB893, PSMB894, PSMB895, PSMB896, PSMB897, PSMB898, PSMB899, PSMHB49SC1133_011A11_1, PSMB896-G100A, PSMA_P72_A10-HC-G54E, PSMA_P72_D01-HC-D95E, PSMA_P72_F01, PSMA_P75_F01, PSMA_P72_F07, PSMA_P70_F02, PSMA_P72_E07, PSMA_P72_D01, PSMA_P72 COL PSMA_P72_A10, PSMA_P72_F02, PSMA_P72_G02, or PSMA_P72_A11. In a specific embodiment, the VH CDR1-3 and VL CDR1-3 are of a single PSMA antibody clone. In certain embodiments, the multispecific PSMA antibody is a multispecific PSMAxCD3 antibody. In specific embodiments, the multispecific PSMAxCD3 antibody is a bispecific PSMAxCD3 antibody. In some embodiments, the second binding domain that binds CD3 comprises the VH CDR1-3 of CD3B376. In some embodiments, the second binding domain that binds CD3 comprises the VL CDR1-3 of CD3B376. In some embodiments, the second binding domain that binds CD3 comprises both the VH CDR1-3 and VL CDR1-3 of CD3B376. In some embodiments, the second binding domain that binds CD3 comprises the VH CDR1-3 of CD3B450. In some embodiments, the second binding domain that binds CD3 comprises the VL CDR1-3 of CD3B450. In some embodiments, the second binding domain that binds CD3 comprises the both the VH CDR1-3 and VL CDR1-3 of CD3B450. In some embodiments, the second binding domain that binds CD3 comprises the VH CDR1-3 of CD3W245. In some embodiments, the second binding domain that binds CD3 comprises the VL CDR1-3 of CD3W245. In some embodiments, the second binding domain that binds CD3 comprises both the VH CDR1-3 and VL CDR1-3 of CD3W245. In some embodiments, the second binding domain that binds CD3 comprises the VH CDR1-3 of CD3B2030. In some embodiments, the second binding domain that binds CD3 comprises the VL CDR1-3 of CD3B2030. In some embodiments, the second binding domain that binds CD3 comprises both the VH CDR1-3 and VL CDR1-3 of CD3B2030.

In another aspect, provided herein is a multispecific antibody that binds PSMA. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific PSMA antibody comprises: (a) a first binding domain that binds PSMA, and (b) a second binding domain that binds to a second target. In one embodiment, the multispecific PSMA antibody comprises: (a) a first binding domain that binds PSMA, and (b) a second binding domain that binds to a second target, and (c) a third binding domain that binds to a third target. In one embodiment, the multispecific PSMA antibody comprises: (a) a first binding domain that binds PSMA, and (b) a second binding domain that binds to a second target, (c) a third binding domain that binds to a third target, and (d) a fourth binding domain that binds to a fourth target.

In another aspect, provided herein is a multispecific antibody comprising: (a) a first binding domain that binds to PSMA, and (b) a second binding domain that binds to a second target that is not PSMA. In another aspect, provided herein is a multispecific antibody comprising: (a) a first binding domain that binds to PSMA, and (b) a second binding domain that binds to CD3. In certain embodiments, the multispecific antibody is a bispecific antibody that comprises: (a) a first binding domain that binds to PSMA, and (b) a second binding domain that binds to CD3. Exemplary first binding domains that bind to PSMA are provided herein. Exemplary second binding domains that bind to CD3 are also provided herein. Also contemplated is any combination of (a) a first binding domain that binds PSMA provided herein, and (b) aa second binding domain that binds CD3 provided herein.

In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814.

In some embodiments, the VH CDR1, VH CDR2, VH CDR3, VL CDR1, VL CDR2, and VL CDR3 amino acid sequences of the first binding domain that binds PSMA are according to the Kabat numbering system. In some embodiments, the VH CDR1, VH CDR2, VH CDR3, VL CDR1, VL CDR2, and VL CDR3 amino acid sequences of the first binding domain that binds PSMA are according to the Chothia numbering system. In some embodiments, the VH CDR1, VH CDR2, VH CDR3, VL CDR1, VL CDR2, and VL CDR3 amino acid sequences of the first binding domain that binds PSMA are according to the AbM numbering system. In some embodiments, the VH CDR1, VH CDR2, VH CDR3, VL CDR1, VL CDR2, and VL CDR3 amino acid sequences of the first binding domain that binds PSMA are according to the Contact numbering system. In some embodiments, the VH CDR1, VH CDR2, VH CDR3, VL CDR1, VL CDR2, and VL CDR3 amino acid sequences of the first binding domain that binds PSMA are according to the IMGT numbering system. In some embodiments, the VH CDR1, VH CDR2, VH CDR3, VL CDR1, VL CDR2, and VL CDR3 amino acid sequences of the first binding domain that binds PSMA are according to a combination of the numbering systems provided herein. Exemplary sets of 6 CDRs (VH CDR1-3 and VL CDR1-3) of certain antibody embodiments are provided herein, including in the Examples and Tables 4-12 (PSMA antibodies), Tables 16-22 (CD3 antibodies), and Tables 23-28 (PSMAxCD3 antibodies) herein. Other sets of CDRs are contemplated and within the scope of the antibody embodiments provided herein.

In some embodiments of the multispecific PSMA antibodies provided herein, the first binding domain binds a PSMA antigen. In some embodiments, the first binding domain binds a PSMA epitope. In some embodiments, the first binding domain specifically binds to PSMA. In some embodiments, the VH CDR1, VH CDR2, VH CDR3, VL CDR1, VL CDR2 and VL CDR3 of the first binding domain form a binding site for an antigen of the PSMA. In some embodiments, the VH CDR1, VH CDR2, VH CDR3, VL CDR1, VL CDR2 and VL CDR3 of the first binding domain form a binding site for an epitope of the PSMA. In some embodiments, the PSMA is present on the surface of a T cell.

In some embodiments of the multispecific PSMA antibodies provided herein, the second target is not a PSMA antigen. In some embodiments of the multispecific PSMA antibodies provided herein, the third target is not a PSMA antigen. In some embodiments of the multispecific PSMA antibodies provided herein, the fourth target is not a PSMA antigen. In some embodiments of the multispecific PSMA antibodies provided herein, the second target is not a PSMA antigen, and the third target is not a PSMA antigen. In some embodiments of the multispecific PSMA antibodies provided herein, the second target is not a PSMA antigen, and the fourth target is not a PSMA antigen. In some embodiments of the multispecific PSMA antibodies provided herein, the third target is not a PSMA antigen, and the fourth target is not a PSMA antigen. In some embodiments of the multispecific PSMA antibodies provided herein, the second target is not a PSMA antigen, the third target is not a PSMA antigen, and the fourth target is not a PSMA antigen. In some embodiments of the multispecific PSMA antibodies provided herein, the second target is not a PSMA epitope. In some embodiments of the multispecific PSMA antibodies provided herein, the third target is not a PSMA epitope. In some embodiments of the multispecific PSMA antibodies provided herein, the fourth target is not a PSMA epitope. In some embodiments of the multispecific PSMA antibodies provided herein, the second target is not a PSMA epitope, and the third target is not a PSMA epitope. In some embodiments of the multispecific PSMA antibodies provided herein, the second target is not a PSMA epitope, and the fourth target is not a PSMA epitope. In some embodiments of the multispecific PSMA antibodies provided herein, the third target is not a PSMA epitope, and the fourth target is not a PSMA epitope. In some embodiments of the multispecific PSMA antibodies provided herein, the second target is not a PSMA epitope, the third target is not a PSMA epitope, and the fourth target is not a PSMA epitope.

In some embodiments of the multispecific PSMA antibodies provided herein, the second target is CD3 and the multispecific PSMA antibody comprises a second binding domain that binds to CD3.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:817, 818, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:823, 824, and 825, respectively, and (ii) a VL comprising a VL CDR1, VL CDR2, and VL CDR3 having an amino acid sequence of SEQ ID NOs:826, 487, and 828, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:829, 830, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:835, 836, and 837, respectively, and (ii) a VL comprising a VL CDR1, VL CDR2, and VL CDR3 having an amino acid sequence of SEQ ID NOs:838, 839, and 840, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:841, 842, and 843, respectively, and (ii) a VL comprising a VL CDR1, VL CDR2, and VL CDR3 having an amino acid sequence of SEQ ID NOs:844, 487, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:847; and (ii) a VL comprising a VL CDR1, a VL CDR2, and a VL CDR3 having an amino acid sequence of a VL CDR1, a VL CDR2, and a VL CDR3, respectively, of SEQ ID NO:848. In one embodiment of the multispecific PSMA antibodies provided herein, the second binding domain that binds CD3 comprises a VH having an amino acid sequence of SEQ ID NO:847. In one embodiment of the multispecific PSMA antibodies provided herein, the second binding domain that binds CD3 comprises a VL having an amino acid sequence of SEQ ID NO:848. In one embodiment of the multispecific PSMA antibodies provided herein, the second binding domain that binds CD3 comprises a VH having an amino acid sequence of SEQ ID NO:847, and a VL having an amino acid sequence of SEQ ID NO:848. In one embodiment of the multispecific PSMA antibodies provided herein, the second binding domain that binds CD3 comprises a heavy chain having an amino acid sequence of SEQ ID NO:849. In one embodiment of the multispecific PSMA antibodies provided herein, the second binding domain that binds CD3 comprises a light chain having an amino acid sequence of SEQ ID NO:850. In one embodiment of the multispecific PSMA antibodies provided herein, the second binding domain that binds CD3 comprises a heavy chain having an amino acid sequence of SEQ ID NO:849, and a light chain having an amino acid sequence of SEQ ID NO:850. In one embodiment of the multispecific PSMA antibodies provided herein, the second binding domain that binds CD3 comprises a VH having an amino acid sequence having at least 95% identity to an amino acid sequence of SEQ ID NO:847. In one embodiment of the multispecific PSMA antibodies provided herein, the second binding domain that binds CD3 comprises a VL having an amino acid sequence having at least 95% identity to an amino acid sequence of SEQ ID NO:848. In one embodiment of the multispecific PSMA antibodies provided herein, the second binding domain that binds CD3 comprises a VH having an amino acid sequence having at least 95% identity to an amino acid sequence of SEQ ID NO:847, and a VL having an amino acid sequence having at least 95% identity to an amino acid sequence of SEQ ID NO:848. In one embodiment of the multispecific PSMA antibodies provided herein, the second binding domain that binds CD3 comprises a heavy chain having an amino acid sequence having at least 95% identity to an amino acid sequence of SEQ ID NO:849. In one embodiment of the multispecific PSMA antibodies provided herein, the second binding domain that binds CD3 comprises a light chain having an amino acid sequence having at least 95% identity to an amino acid sequence of SEQ ID NO:850. In one embodiment of the multispecific PSMA antibodies provided herein, the second binding domain that binds CD3 comprises a heavy chain having an amino acid sequence having at least 95% identity to an amino acid sequence of SEQ ID NO:849, and a light chain having an amino acid sequence having at least 95% identity to an amino acid sequence of SEQ ID NO:850.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:817, 818, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:823, 824, and 825, respectively, and (ii) a VL comprising a VL CDR1, VL CDR2, and VL CDR3 having an amino acid sequence of SEQ ID NOs:826, 487, and 828, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:829, 830, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:835, 836, and 837, respectively, and (ii) a VL comprising a VL CDR1, VL CDR2, and VL CDR3 having an amino acid sequence of SEQ ID NOs:838, 839, and 840, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:841, 842, and 843, respectively, and (ii) a VL comprising a VL CDR1, VL CDR2, and VL CDR3 having an amino acid sequence of SEQ ID NOs:844, 487, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:847; and (ii) a VL comprising a VL CDR1, a VL CDR2, and a VL CDR3 having an amino acid sequence of a VL CDR1, a VL CDR2, and a VL CDR3, respectively, of SEQ ID NO:848. In one embodiment of the multispecific PSMA antibodies provided herein, the second binding domain that binds CD3 comprises a VH having an amino acid sequence of SEQ ID NO:847. In one embodiment of the multispecific PSMA antibodies provided herein, the second binding domain that binds CD3 comprises a VL having an amino acid sequence of SEQ ID NO:848. In one embodiment of the multispecific PSMA antibodies provided herein, the second binding domain that binds CD3 comprises a VH having an amino acid sequence of SEQ ID NO:847, and a VL having an amino acid sequence of SEQ ID NO:848. In one embodiment of the multispecific PSMA antibodies provided herein, the second binding domain that binds CD3 comprises a heavy chain having an amino acid sequence of SEQ ID NO:883. In one embodiment of the multispecific PSMA antibodies provided herein, the second binding domain that binds CD3 comprises a light chain having an amino acid sequence of SEQ ID NO:850. In one embodiment of the multispecific PSMA antibodies provided herein, the second binding domain that binds CD3 comprises a heavy chain having an amino acid sequence of SEQ ID NO:883, and a light chain having an amino acid sequence of SEQ ID NO:850. In one embodiment of the multispecific PSMA antibodies provided herein, the second binding domain that binds CD3 comprises a VH having an amino acid sequence having at least 95% identity to an amino acid sequence of SEQ ID NO:847. In one embodiment of the multispecific PSMA antibodies provided herein, the second binding domain that binds CD3 comprises a VL having an amino acid sequence having at least 95% identity to an amino acid sequence of SEQ ID NO:848. In one embodiment of the multispecific PSMA antibodies provided herein, the second binding domain that binds CD3 comprises a VH having an amino acid sequence having at least 95% identity to an amino acid sequence of SEQ ID NO:847, and a VL having an amino acid sequence having at least 95% identity to an amino acid sequence of SEQ ID NO:848. In one embodiment of the multispecific PSMA antibodies provided herein, the second binding domain that binds CD3 comprises a heavy chain having an amino acid sequence having at least 95% identity to an amino acid sequence of SEQ ID NO:883. In one embodiment of the multispecific PSMA antibodies provided herein, the second binding domain that binds CD3 comprises a light chain having an amino acid sequence having at least 95% identity to an amino acid sequence of SEQ ID NO:850. In one embodiment of the multispecific PSMA antibodies provided herein, the second binding domain that binds CD3 comprises a heavy chain having an amino acid sequence having at least 95% identity to an amino acid sequence of SEQ ID NO:883, and a light chain having an amino acid sequence having at least 95% identity to an amino acid sequence of SEQ ID NO:850.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:817, 818, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:823, 824, and 825, respectively, and (ii) a VL comprising a VL CDR1, VL CDR2, and VL CDR3 having an amino acid sequence of SEQ ID NOs:826, 487, and 828, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:829, 830, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:835, 836, and 837, respectively, and (ii) a VL comprising a VL CDR1, VL CDR2, and VL CDR3 having an amino acid sequence of SEQ ID NOs:838, 907, and 840, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:841, 842, and 843, respectively, and (ii) a VL comprising a VL CDR1, VL CDR2, and VL CDR3 having an amino acid sequence of SEQ ID NOs:844, 487, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:915; and (ii) a VL comprising a VL CDR1, a VL CDR2, and a VL CDR3 having an amino acid sequence of a VL CDR1, a VL CDR2, and a VL CDR3, respectively, of SEQ ID NO:916. In one embodiment of the multispecific PSMA antibodies provided herein, the second binding domain that binds CD3 comprises a VH having an amino acid sequence of SEQ ID NO:915. In one embodiment of the multispecific PSMA antibodies provided herein, the second binding domain that binds CD3 comprises a VL having an amino acid sequence of SEQ ID NO:916. In one embodiment of the multispecific PSMA antibodies provided herein, the second binding domain that binds CD3 comprises a VH having an amino acid sequence of SEQ ID NO:915, and a VL having an amino acid sequence of SEQ ID NO:916. In one embodiment of the multispecific PSMA antibodies provided herein, the second binding domain that binds CD3 comprises a heavy chain having an amino acid sequence of SEQ ID NO:917. In one embodiment of the multispecific PSMA antibodies provided herein, the second binding domain that binds CD3 comprises a light chain having an amino acid sequence of SEQ ID NO:918. In one embodiment of the multispecific PSMA antibodies provided herein, the second binding domain that binds CD3 comprises a heavy chain having an amino acid sequence of SEQ ID NO:917, and a light chain having an amino acid sequence of SEQ ID NO:918. In one embodiment of the multispecific PSMA antibodies provided herein, the second binding domain that binds CD3 comprises a VH having an amino acid sequence having at least 95% identity to an amino acid sequence of SEQ ID NO:915. In one embodiment of the multispecific PSMA antibodies provided herein, the second binding domain that binds CD3 comprises a VL having an amino acid sequence having at least 95% identity to an amino acid sequence of SEQ ID NO:916. In one embodiment of the multispecific PSMA antibodies provided herein, the second binding domain that binds CD3 comprises a VH having an amino acid sequence having at least 95% identity to an amino acid sequence of SEQ ID NO:915, and a VL having an amino acid sequence having at least 95% identity to an amino acid sequence of SEQ ID NO:916. In one embodiment of the multispecific PSMA antibodies provided herein, the second binding domain that binds CD3 comprises a heavy chain having an amino acid sequence having at least 95% identity to an amino acid sequence of SEQ ID NO:917. In one embodiment of the multispecific PSMA antibodies provided herein, the second binding domain that binds CD3 comprises a light chain having an amino acid sequence having at least 95% identity to an amino acid sequence of SEQ ID NO:918. In one embodiment of the multispecific PSMA antibodies provided herein, the second binding domain that binds CD3 comprises a heavy chain having an amino acid sequence having at least 95% identity to an amino acid sequence of SEQ ID NO:917, and a light chain having an amino acid sequence having at least 95% identity to an amino acid sequence of SEQ ID NO:918.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:817, 818, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:823, 824, and 825, respectively, and (ii) a VL comprising a VL CDR1, VL CDR2, and VL CDR3 having an amino acid sequence of SEQ ID NOs:826, 487, and 828, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:829, 830, and 819, respectively, and (ii) a VL comprising a VL CDR1, VL CDR2, and VL CDR3 having an amino acid sequence of SEQ ID NOs:820, 821, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:835, 836, and 837, respectively, and (ii) a VL comprising a VL CDR1, VL CDR2, and VL CDR3 having an amino acid sequence of SEQ ID NOs:838, 907, and 840, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:841, 842, and 843, respectively, and (ii) a VL comprising a VL CDR1, VL CDR2, and VL CDR3 having an amino acid sequence of SEQ ID NOs:844, 487, and 822, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:915; and (ii) a VL comprising a VL CDR1, a VL CDR2, and a VL CDR3 having an amino acid sequence of a VL CDR1, a VL CDR2, and a VL CDR3, respectively, of SEQ ID NO:916. In one embodiment of the multispecific PSMA antibodies provided herein, the second binding domain that binds CD3 comprises a VH having an amino acid sequence of SEQ ID NO:915. In one embodiment of the multispecific PSMA antibodies provided herein, the second binding domain that binds CD3 comprises a VL having an amino acid sequence of SEQ ID NO:916. In one embodiment of the multispecific PSMA antibodies provided herein, the second binding domain that binds CD3 comprises a VH having an amino acid sequence of SEQ ID NO:915, and a VL having an amino acid sequence of SEQ ID NO:916. In one embodiment of the multispecific PSMA antibodies provided herein, the second binding domain that binds CD3 comprises a heavy chain having an amino acid sequence of SEQ ID NO:951. In one embodiment of the multispecific PSMA antibodies provided herein, the second binding domain that binds CD3 comprises a light chain having an amino acid sequence of SEQ ID NO:918. In one embodiment of the multispecific PSMA antibodies provided herein, the second binding domain that binds CD3 comprises a heavy chain having an amino acid sequence of SEQ ID NO:951, and a light chain having an amino acid sequence of SEQ ID NO:918. In one embodiment of the multispecific PSMA antibodies provided herein, the second binding domain that binds CD3 comprises a VH having an amino acid sequence having at least 95% identity to an amino acid sequence of SEQ ID NO:915. In one embodiment of the multispecific PSMA antibodies provided herein, the second binding domain that binds CD3 comprises a VL having an amino acid sequence having at least 95% identity to an amino acid sequence of SEQ ID NO:916. In one embodiment of the multispecific PSMA antibodies provided herein, the second binding domain that binds CD3 comprises a VH having an amino acid sequence having at least 95% identity to an amino acid sequence of SEQ ID NO:915, and a VL having an amino acid sequence having at least 95% identity to an amino acid sequence of SEQ ID NO:916. In one embodiment of the multispecific PSMA antibodies provided herein, the second binding domain that binds CD3 comprises a heavy chain having an amino acid sequence having at least 95% identity to an amino acid sequence of SEQ ID NO:951. In one embodiment of the multispecific PSMA antibodies provided herein, the second binding domain that binds CD3 comprises a light chain having an amino acid sequence having at least 95% identity to an amino acid sequence of SEQ ID NO:918. In one embodiment of the multispecific PSMA antibodies provided herein, the second binding domain that binds CD3 comprises a heavy chain having an amino acid sequence having at least 95% identity to an amino acid sequence of SEQ ID NO:951, and a light chain having an amino acid sequence having at least 95% identity to an amino acid sequence of SEQ ID NO:918.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1, 954, and 955, respectively, and (ii) a VL comprising a VL CDR1, VL CDR2, and VL CDR3 having an amino acid sequence of SEQ ID NOs:956, 957, and 958, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:7, 960, and 961, respectively, and (ii) a VL comprising a VL CDR1, VL CDR2, and VL CDR3 having an amino acid sequence of SEQ ID NOs:962, 963, and 964, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:13, 966, and 955, respectively, and (ii) a VL comprising a VL CDR1, VL CDR2, and VL CDR3 having an amino acid sequence of SEQ ID NOs:956, 957, and 958, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:19, 972, and 973, respectively, and (ii) a VL comprising a VL CDR1, VL CDR2, and VL CDR3 having an amino acid sequence of SEQ ID NOs:974, 975, and 976, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:25, 978, and 979, respectively, and (ii) a VL comprising a VL CDR1, VL CDR2, and VL CDR3 having an amino acid sequence of SEQ ID NOs:980, 963, and 958, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:983; and (ii) a VL comprising a VL CDR1, a VL CDR2, and a VL CDR3 having an amino acid sequence of a VL CDR1, a VL CDR2, and a VL CDR3, respectively, of SEQ ID NO:984. In one embodiment of the multispecific PSMA antibodies provided herein, the second binding domain that binds CD3 comprises a VH having an amino acid sequence of SEQ ID NO:983. In one embodiment of the multispecific PSMA antibodies provided herein, the second binding domain that binds CD3 comprises a VL having an amino acid sequence of SEQ ID NO:984. In one embodiment of the multispecific PSMA antibodies provided herein, the second binding domain that binds CD3 comprises a VH having an amino acid sequence of SEQ ID NO:983, and a VL having an amino acid sequence of SEQ ID NO:984. In one embodiment of the multispecific PSMA antibodies provided herein, the second binding domain that binds CD3 comprises a heavy chain having an amino acid sequence of SEQ ID NO:985. In one embodiment of the multispecific PSMA antibodies provided herein, the second binding domain that binds CD3 comprises a light chain having an amino acid sequence of SEQ ID NO:986. In one embodiment of the multispecific PSMA antibodies provided herein, the second binding domain that binds CD3 comprises a heavy chain having an amino acid sequence of SEQ ID NO:985, and a light chain having an amino acid sequence of SEQ ID NO:986. In one embodiment of the multispecific PSMA antibodies provided herein, the second binding domain that binds CD3 comprises a VH having an amino acid sequence having at least 95% identity to an amino acid sequence of SEQ ID NO:983. In one embodiment of the multispecific PSMA antibodies provided herein, the second binding domain that binds CD3 comprises a VL having an amino acid sequence having at least 95% identity to an amino acid sequence of SEQ ID NO:984. In one embodiment of the multispecific PSMA antibodies provided herein, the second binding domain that binds CD3 comprises a VH having an amino acid sequence having at least 95% identity to an amino acid sequence of SEQ ID NO:983, and a VL having an amino acid sequence having at least 95% identity to an amino acid sequence of SEQ ID NO:984. In one embodiment of the multispecific PSMA antibodies provided herein, the second binding domain that binds CD3 comprises a heavy chain having an amino acid sequence having at least 95% identity to an amino acid sequence of SEQ ID NO:985. In one embodiment of the multispecific PSMA antibodies provided herein, the second binding domain that binds CD3 comprises a light chain having an amino acid sequence having at least 95% identity to an amino acid sequence of SEQ ID NO:986. In one embodiment of the multispecific PSMA antibodies provided herein, the second binding domain that binds CD3 comprises a heavy chain having an amino acid sequence having at least 95% identity to an amino acid sequence of SEQ ID NO:985, and a light chain having an amino acid sequence having at least 95% identity to an amino acid sequence of SEQ ID NO:986.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1, 954, and 955, respectively, and (ii) a VL comprising a VL CDR1, VL CDR2, and VL CDR3 having an amino acid sequence of SEQ ID NOs:956, 957, and 958, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:7, 960, and 961, respectively, and (ii) a VL comprising a VL CDR1, VL CDR2, and VL CDR3 having an amino acid sequence of SEQ ID NOs:962, 963, and 964, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:13, 966, and 955, respectively, and (ii) a VL comprising a VL CDR1, VL CDR2, and VL CDR3 having an amino acid sequence of SEQ ID NOs:956, 957, and 958, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:19, 972, and 973, respectively, and (ii) a VL comprising a VL CDR1, VL CDR2, and VL CDR3 having an amino acid sequence of SEQ ID NOs:974, 975, and 976, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:25, 978, and 979, respectively, and (ii) a VL comprising a VL CDR1, VL CDR2, and VL CDR3 having an amino acid sequence of SEQ ID NOs:980, 963, and 958, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:983; and (ii) a VL comprising a VL CDR1, a VL CDR2, and a VL CDR3 having an amino acid sequence of a VL CDR1, a VL CDR2, and a VL CDR3, respectively, of SEQ ID NO:984. In one embodiment of the multispecific PSMA antibodies provided herein, the second binding domain that binds CD3 comprises a VH having an amino acid sequence of SEQ ID NO:983. In one embodiment of the multispecific PSMA antibodies provided herein, the second binding domain that binds CD3 comprises a VL having an amino acid sequence of SEQ ID NO:984. In one embodiment of the multispecific PSMA antibodies provided herein, the second binding domain that binds CD3 comprises a VH having an amino acid sequence of SEQ ID NO:983, and a VL having an amino acid sequence of SEQ ID NO:984. In one embodiment of the multispecific PSMA antibodies provided herein, the second binding domain that binds CD3 comprises a heavy chain having an amino acid sequence of SEQ ID NO:1019. In one embodiment of the multispecific PSMA antibodies provided herein, the second binding domain that binds CD3 comprises a light chain having an amino acid sequence of SEQ ID NO:986. In one embodiment of the multispecific PSMA antibodies provided herein, the second binding domain that binds CD3 comprises a heavy chain having an amino acid sequence of SEQ ID NO:1019, and a light chain having an amino acid sequence of SEQ ID NO:986. In one embodiment of the multispecific PSMA antibodies provided herein, the second binding domain that binds CD3 comprises a VH having an amino acid sequence having at least 95% identity to an amino acid sequence of SEQ ID NO:983. In one embodiment of the multispecific PSMA antibodies provided herein, the second binding domain that binds CD3 comprises a VL having an amino acid sequence having at least 95% identity to an amino acid sequence of SEQ ID NO:984. In one embodiment of the multispecific PSMA antibodies provided herein, the second binding domain that binds CD3 comprises a VH having an amino acid sequence having at least 95% identity to an amino acid sequence of SEQ ID NO:983, and a VL having an amino acid sequence having at least 95% identity to an amino acid sequence of SEQ ID NO:984. In one embodiment of the multispecific PSMA antibodies provided herein, the second binding domain that binds CD3 comprises a heavy chain having an amino acid sequence having at least 95% identity to an amino acid sequence of SEQ ID NO:1019. In one embodiment of the multispecific PSMA antibodies provided herein, the second binding domain that binds CD3 comprises a light chain having an amino acid sequence having at least 95% identity to an amino acid sequence of SEQ ID NO:986. In one embodiment of the multispecific PSMA antibodies provided herein, the second binding domain that binds CD3 comprises a heavy chain having an amino acid sequence having at least 95% identity to an amino acid sequence of SEQ ID NO:1019, and a light chain having an amino acid sequence having at least 95% identity to an amino acid sequence of SEQ ID NO:986.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1467, 1468, and 1469, respectively, and (ii) a VL comprising a VL CDR1, VL CDR2, and VL CDR3 having an amino acid sequence of SEQ ID NOs:1470, 1471, and 1472, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1473, 1474, and 1475, respectively, and (ii) a VL comprising a VL CDR1, VL CDR2, and VL CDR3 having an amino acid sequence of SEQ ID NOs:1476, 1477, and 1478, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1479, 1480, and 1481, respectively, and (ii) a VL comprising a VL CDR1, VL CDR2, and VL CDR3 having an amino acid sequence of SEQ ID NOs:1482, 1477, and 1472, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1508, 1509, and 1469, respectively, and (ii) a VL comprising a VL CDR1, VL CDR2, and VL CDR3 having an amino acid sequence of SEQ ID NOs:1470, 1471, and 1472, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1510, 1511, and 1512, respectively, and (ii) a VL comprising a VL CDR1, VL CDR2, and VL CDR3 having an amino acid sequence of SEQ ID NOs:1513, 1514, and 1515, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:1463; and (ii) a VL comprising a VL CDR1, a VL CDR2, and a VL CDR3 having an amino acid sequence of a VL CDR1, a VL CDR2, and a VL CDR3, respectively, of SEQ ID NO:1464. In one embodiment of the multispecific PSMA antibodies provided herein, the second binding domain that binds CD3 comprises a VH having an amino acid sequence of SEQ ID NO:1463. In one embodiment of the multispecific PSMA antibodies provided herein, the second binding domain that binds CD3 comprises a VL having an amino acid sequence of SEQ ID NO:1464. In one embodiment of the multispecific PSMA antibodies provided herein, the second binding domain that binds CD3 comprises a VH having an amino acid sequence of SEQ ID NO:1463, and a VL having an amino acid sequence of SEQ ID NO:1464. In one embodiment of the multispecific PSMA antibodies provided herein, the second binding domain that binds CD3 comprises a VH having an amino acid sequence having at least 95% identity to an amino acid sequence of SEQ ID NO:1463. In one embodiment of the multispecific PSMA antibodies provided herein, the second binding domain that binds CD3 comprises a VL having an amino acid sequence having at least 95% identity to an amino acid sequence of SEQ ID NO:1464. In one embodiment of the multispecific PSMA antibodies provided herein, the second binding domain that binds CD3 comprises a VH having an amino acid sequence having at least 95% identity to an amino acid sequence of SEQ ID NO:1463, and a VL having an amino acid sequence having at least 95% identity to an amino acid sequence of SEQ ID NO:1464.

In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1467, 1468, and 1506, respectively, and (ii) a VL comprising a VL CDR1, VL CDR2, and VL CDR3 having an amino acid sequence of SEQ ID NOs:1470, 1471, and 1472, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1473, 1474, and 1507, respectively, and (ii) a VL comprising a VL CDR1, VL CDR2, and VL CDR3 having an amino acid sequence of SEQ ID NOs:1476, 1477, and 1478, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1479, 1480, and 1518, respectively, and (ii) a VL comprising a VL CDR1, VL CDR2, and VL CDR3 having an amino acid sequence of SEQ ID NOs:1482, 1477, and 1472, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1508, 1509, and 1506, respectively, and (ii) a VL comprising a VL CDR1, VL CDR2, and VL CDR3 having an amino acid sequence of SEQ ID NOs:1470, 1471, and 1472, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of SEQ ID NOs:1516, 1511, and 1517, respectively, and (ii) a VL comprising a VL CDR1, VL CDR2, and VL CDR3 having an amino acid sequence of SEQ ID NOs:1513, 1514, and 1515, respectively. In one embodiment of the multispecific PSMA antibodies provided herein, the second binding domain that binds CD3, comprises: (i) a VH comprising a VH CDR1, a VH CDR2, and a VH CDR3 having an amino acid sequence of a VH CDR1, a VH CDR2, and a VH CDR3, respectively, of SEQ ID NO:1505; and (ii) a VL comprising a VL CDR1, a VL CDR2, and a VL CDR3 having an amino acid sequence of a VL CDR1, a VL CDR2, and a VL CDR3, respectively, of SEQ ID NO:1464. In one embodiment of the multispecific PSMA antibodies provided herein, the second binding domain that binds CD3 comprises a VH having an amino acid sequence of SEQ ID NO:1505. In one embodiment of the multispecific PSMA antibodies provided herein, the second binding domain that binds CD3 comprises a VL having an amino acid sequence of SEQ ID NO:1464. In one embodiment of the multispecific PSMA antibodies provided herein, the second binding domain that binds CD3 comprises a VH having an amino acid sequence of SEQ ID NO:1505, and a VL having an amino acid sequence of SEQ ID NO:1464. In one embodiment of the multispecific PSMA antibodies provided herein, the second binding domain that binds CD3 comprises a VH having an amino acid sequence having at least 95% identity to an amino acid sequence of SEQ ID NO:1505. In one embodiment of the multispecific PSMA antibodies provided herein, the second binding domain that binds CD3 comprises a VL having an amino acid sequence having at least 95% identity to an amino acid sequence of SEQ ID NO:1464. In one embodiment of the multispecific PSMA antibodies provided herein, the second binding domain that binds CD3 comprises a VH having an amino acid sequence having at least 95% identity to an amino acid sequence of SEQ ID NO:1505, and a VL having an amino acid sequence having at least 95% identity to an amino acid sequence of SEQ ID NO:1464.

In some embodiments, the second binding domain that binds to CD3 comprises a scFv comprising a VH CDR1, a VH CDR2, a VH CDR3, a VL CDR1, a VL CDR2, a VL CDR3 having an amino acid sequence of a VH CDR1, a VH CDR2, a VH CDR3, a VL CDR1, a VL CDR2, a VL CDR3 respectively, of a scFv having an amino acid sequence of SEQ ID NO:1524. In one embodiment of the multispecific PSMA antibodies provided herein, the second binding domain that binds CD3 comprises an scFv having an amino acid sequence of SEQ ID NO:1524. In one embodiment of the multispecific PSMA antibodies provided herein, the second binding domain that binds CD3 comprises an scFv having an amino acid sequence having at least 95% identity to an amino acid sequence of SEQ ID NO:1524.

In some embodiments of the multispecific PSMAxCD3 antibody provided herein, the first binding domain that binds PSMA comprises a heavy chain (HC) having an amino acid sequence at least 80% identical to the amino acid sequence of SEQ ID NO:33. In some embodiments of the multispecific antibody provided herein, the first binding domain that binds PSMA comprises a HC having an amino acid sequence at least 80% identical to the amino acid sequence of SEQ ID NO:101. In some embodiments of the multispecific antibody provided herein, the first binding domain that binds PSMA comprises a HC having an amino acid sequence at least 80% identical to the amino acid sequence of SEQ ID NO:169. In some embodiments of the multispecific antibody provided herein, the first binding domain that binds PSMA comprises a HC having an amino acid sequence at least 80% identical to the amino acid sequence of SEQ ID NO:237. In some embodiments of the multispecific antibody provided herein, the first binding domain that binds PSMA comprises a HC having an amino acid sequence at least 80% identical to the amino acid sequence of SEQ ID NO:305. In some embodiments of the multispecific antibody provided herein, the first binding domain that binds PSMA comprises a HC having an amino acid sequence at least 80% identical to the amino acid sequence of SEQ ID NO:373. In some embodiments of the multispecific antibody provided herein, the first binding domain that binds PSMA comprises a HC having an amino acid sequence at least 80% identical to the amino acid sequence of SEQ ID NO:407. In some embodiments of the multispecific antibody provided herein, the first binding domain that binds PSMA comprises a HC having an amino acid sequence at least 80% identical to the amino acid sequence of SEQ ID NO:441. In some embodiments of the multispecific antibody provided herein, the first binding domain that binds PSMA comprises a HC having an amino acid sequence at least 80% identical to the amino acid sequence of SEQ ID NO:1242. In some embodiments of the multispecific antibody provided herein, the first binding domain that binds PSMA comprises a HC having an amino acid sequence at least 80% identical to the amino acid sequence of SEQ ID NO:1244. In some embodiments of the multispecific antibody provided herein, the first binding domain that binds PSMA comprises a HC having an amino acid sequence at least 80% identical to the amino acid sequence of SEQ ID NO:1248. In some embodiments of the multispecific antibody provided herein, the first binding domain that binds PSMA comprises a HC having an amino acid sequence at least 80% identical to the amino acid sequence of SEQ ID NO:1250. In some embodiments of the multispecific antibody provided herein, the first binding domain that binds PSMA comprises a HC having an amino acid sequence at least 80% identical to the amino acid sequence of SEQ ID NO:1252. In some embodiments of the multispecific antibody provided herein, the first binding domain that binds PSMA comprises a HC having an amino acid sequence at least 80% identical to the amino acid sequence of SEQ ID NO:1254. In some embodiments of the multispecific antibody provided herein, the first binding domain that binds PSMA comprises a HC having an amino acid sequence at least 80% identical to the amino acid sequence of SEQ ID NO:1256. In some embodiments of the multispecific antibody provided herein, the first binding domain that binds PSMA comprises a HC having an amino acid sequence at least 80% identical to the amino acid sequence of SEQ ID NO:1258. In some embodiments of the multispecific antibody provided herein, the first binding domain that binds PSMA comprises a HC having an amino acid sequence at least 80% identical to the amino acid sequence of SEQ ID NO:1260. In some embodiments of the multispecific antibody provided herein, the first binding domain that binds PSMA comprises a HC having an amino acid sequence at least 80% identical to the amino acid sequence of SEQ ID NO:1262. In some embodiments of the multispecific antibody provided herein, the first binding domain that binds PSMA comprises a HC having an amino acid sequence at least 80% identical to the amino acid sequence of SEQ ID NO:1264. In some embodiments of the multispecific antibody provided herein, the first binding domain that binds PSMA comprises a HC having an amino acid sequence at least 80% identical to the amino acid sequence of SEQ ID NO:1266. In some embodiments of the multispecific antibody provided herein, the first binding domain that binds PSMA comprises a HC having an amino acid sequence at least 80% identical to the amino acid sequence of SEQ ID NO:1268. In some embodiments of the multispecific antibody provided herein, the first binding domain that binds PSMA comprises a HC having an amino acid sequence at least 80% identical to the amino acid sequence of SEQ ID NO:1270.

In some embodiments of the multispecific PSMAxCD3 antibody provided herein, the first binding domain that binds PSMA comprises a light chain (LC) having an amino acid sequence at least 80% identical to the amino acid sequence of SEQ ID NO:34. In some embodiments of the multispecific antibody provided herein, the first binding domain that binds PSMA comprises a LC having an amino acid sequence at least 80% identical to the amino acid sequence of SEQ ID NO:68. In some embodiments of the multispecific antibody provided herein, the first binding domain that binds PSMA comprises a LC having an amino acid sequence at least 80% identical to the amino acid sequence of SEQ ID NO:102. In some embodiments of the multispecific antibody provided herein, the first binding domain that binds PSMA comprises a LC having an amino acid sequence at least 80% identical to the amino acid sequence of SEQ ID NO:136. In some embodiments of the multispecific antibody provided herein, the first binding domain that binds PSMA comprises a LC having an amino acid sequence at least 80% identical to the amino acid sequence of SEQ ID NO:238. In some embodiments of the multispecific antibody provided herein, the first binding domain that binds PSMA comprises a LC having an amino acid sequence at least 80% identical to the amino acid sequence of SEQ ID NO:272. In some embodiments of the multispecific antibody provided herein, the first binding domain that binds PSMA comprises a LC having an amino acid sequence at least 80% identical to the amino acid sequence of SEQ ID NO:374. In some embodiments of the multispecific antibody provided herein, the first binding domain that binds PSMA comprises a LC having an amino acid sequence at least 80% identical to the amino acid sequence of SEQ ID NO:408.

In some embodiments of the multispecific PSMAxCD3 antibody provided herein, the first binding domain that binds PSMA comprises a scFv having an amino acid sequence of SEQ ID NO:1485. In some embodiments of the multispecific antibody provided herein, the first binding domain that binds PSMA comprises a scFv having an amino acid sequence of SEQ ID NO:1486. In some embodiments of the multispecific antibody provided herein, the first binding domain that binds PSMA comprises a scFv having an amino acid sequence of SEQ ID NO:1487. In some embodiments of the multispecific antibody provided herein, the first binding domain that binds PSMA comprises a scFv having an amino acid sequence of SEQ ID NO:1488. In some embodiments of the multispecific antibody provided herein, the first binding domain that binds PSMA comprises a scFv having an amino acid sequence of SEQ ID NO:1489. In some embodiments of the multispecific antibody provided herein, the first binding domain that binds PSMA comprises a scFv having an amino acid sequence of SEQ ID NO:1490. In some embodiments of the multispecific antibody provided herein, the first binding domain that binds PSMA comprises a scFv having an amino acid sequence of SEQ ID NO:1491. In some embodiments of the multispecific antibody provided herein, the first binding domain that binds PSMA comprises a scFv having an amino acid sequence of SEQ ID NO:1492. In some embodiments of the multispecific antibody provided herein, the first binding domain that binds PSMA comprises a scFv having an amino acid sequence of SEQ ID NO:1493. In some embodiments of the multispecific antibody provided herein, the first binding domain that binds PSMA comprises a scFv having an amino acid sequence of SEQ ID NO:1494. In some embodiments of the multispecific antibody provided herein, the first binding domain that binds PSMA comprises a scFv having an amino acid sequence of SEQ ID NO:1495. In some embodiments of the multispecific antibody provided herein, the first binding domain that binds PSMA comprises a scFv having an amino acid sequence of SEQ ID NO:1496. In some embodiments of the multispecific antibody provided herein, the first binding domain that binds PSMA comprises a scFv having an amino acid sequence of SEQ ID NO:1497. In some embodiments of the multispecific antibody provided herein, the first binding domain that binds PSMA comprises a scFv having an amino acid sequence of SEQ ID NO:1498. In some embodiments of the multispecific antibody provided herein, the first binding domain that binds PSMA comprises a scFv having an amino acid sequence of SEQ ID NO:1499. In some embodiments of the multispecific antibody provided herein, the first binding domain that binds PSMA comprises a scFv having an amino acid sequence of SEQ ID NO:1500. In some embodiments of the multispecific antibody provided herein, the first binding domain that binds PSMA comprises a scFv having an amino acid sequence of SEQ ID NO:1526. In some embodiments of the multispecific antibody provided herein, the first binding domain that binds PSMA comprises a scFv having an amino acid sequence of SEQ ID NO:1527. In some embodiments of the multispecific antibody provided herein, the first binding domain that binds PSMA comprises a scFv having an amino acid sequence of SEQ ID NO:1528. In some embodiments of the multispecific antibody provided herein, the first binding domain that binds PSMA comprises a scFv having an amino acid sequence of SEQ ID NO:1529. In some embodiments of the multispecific antibody provided herein, the first binding domain that binds PSMA comprises a scFv having an amino acid sequence of SEQ ID NO:1530. In some embodiments of the multispecific antibody provided herein, the first binding domain that binds PSMA comprises a scFv having an amino acid sequence of SEQ ID NO:1531.

In some embodiments of the multispecific PSMAxCD3 antibody provided herein, the first binding domain that binds PSMA comprises a scFv having an amino acid sequence at least 80% identical to the amino acid sequence of SEQ ID NO:1485. In some embodiments of the multispecific antibody provided herein, the first binding domain that binds PSMA comprises a scFv having an amino acid sequence at least 80% identical to the amino acid sequence of SEQ ID NO:1486. In some embodiments of the multispecific antibody provided herein, the first binding domain that binds PSMA comprises a scFv having an amino acid sequence at least 80% identical to the amino acid sequence of SEQ ID NO:1487. In some embodiments of the multispecific antibody provided herein, the first binding domain that binds PSMA comprises a scFv having an amino acid sequence at least 80% identical to the amino acid sequence of SEQ ID NO:1488. In some embodiments of the multispecific antibody provided herein, the first binding domain that binds PSMA comprises a scFv having an amino acid sequence at least 80% identical to the amino acid sequence of SEQ ID NO:1489. In some embodiments of the multispecific antibody provided herein, the first binding domain that binds PSMA comprises a scFv having an amino acid sequence at least 80% identical to the amino acid sequence of SEQ ID NO:1490. In some embodiments of the multispecific antibody provided herein, the first binding domain that binds PSMA comprises a scFv having an amino acid sequence at least 80% identical to the amino acid sequence of SEQ ID NO:1491. In some embodiments of the multispecific antibody provided herein, the first binding domain that binds PSMA comprises a scFv having an amino acid sequence at least 80% identical to the amino acid sequence of SEQ ID NO:1492. In some embodiments of the multispecific antibody provided herein, the first binding domain that binds PSMA comprises a scFv having an amino acid sequence at least 80% identical to the amino acid sequence of SEQ ID NO:1493. In some embodiments of the multispecific antibody provided herein, the first binding domain that binds PSMA comprises a scFv having an amino acid sequence at least 80% identical to the amino acid sequence of SEQ ID NO:1494. In some embodiments of the multispecific antibody provided herein, the first binding domain that binds PSMA comprises a scFv having an amino acid sequence at least 80% identical to the amino acid sequence of SEQ ID NO:1495. In some embodiments of the multispecific antibody provided herein, the first binding domain that binds PSMA comprises a scFv having an amino acid sequence at least 80% identical to the amino acid sequence of SEQ ID NO:1496. In some embodiments of the multispecific antibody provided herein, the first binding domain that binds PSMA comprises a scFv having an amino acid sequence at least 80% identical to the amino acid sequence of SEQ ID NO:1497. In some embodiments of the multispecific antibody provided herein, the first binding domain that binds PSMA comprises a scFv having an amino acid sequence at least 80% identical to the amino acid sequence of SEQ ID NO:1498. In some embodiments of the multispecific antibody provided herein, the first binding domain that binds PSMA comprises a scFv having an amino acid sequence at least 80% identical to the amino acid sequence of SEQ ID NO:1499. In some embodiments of the multispecific antibody provided herein, the first binding domain that binds PSMA comprises a scFv having an amino acid sequence at least 80% identical to the amino acid sequence of SEQ ID NO:1500. In some embodiments of the multispecific antibody provided herein, the first binding domain that binds PSMA comprises a scFv having an amino acid sequence at least 80% identical to the amino acid sequence of SEQ ID NO:1526. In some embodiments of the multispecific antibody provided herein, the first binding domain that binds PSMA comprises a scFv having an amino acid sequence at least 80% identical to the amino acid sequence of SEQ ID NO:1527. In some embodiments of the multispecific antibody provided herein, the first binding domain that binds PSMA comprises a scFv having an amino acid sequence at least 80% identical to the amino acid sequence of SEQ ID NO:1528. In some embodiments of the multispecific antibody provided herein, the first binding domain that binds PSMA comprises a scFv having an amino acid sequence at least 80% identical to the amino acid sequence of SEQ ID NO:1529. In some embodiments of the multispecific antibody provided herein, the first binding domain that binds PSMA comprises a scFv having an amino acid sequence at least 80% identical to the amino acid sequence of SEQ ID NO:1530. In some embodiments of the multispecific antibody provided herein, the first binding domain that binds PSMA comprises a scFv having an amino acid sequence at least 80% identical to the amino acid sequence of SEQ ID NO:1531.

In some embodiments of the multispecific PSMAxCD3 antibody provided herein, the first binding domain that binds PSMA comprises (i) a HC having an amino acid sequence of SEQ ID NO:441; and (ii) a LC having an amino acid sequence of SEQ ID NO:272.

In some embodiments of the multispecific PSMAxCD3 antibody provided herein, the second binding domain that binds CD3 comprises a heavy chain (HC) having an amino acid sequence at least 80% identical to the amino acid sequence of SEQ ID NO:849. In some embodiments of the multispecific antibody provided herein, the second binding domain that binds CD3 comprises a HC having an amino acid sequence at least 80% identical to the amino acid sequence of SEQ ID NO:883. In some embodiments of the multispecific antibody provided herein, the second binding domain that binds CD3 comprises a HC having an amino acid sequence at least 80% identical to the amino acid sequence of SEQ ID NO:917. In some embodiments of the multispecific antibody provided herein, the second binding domain that binds CD3 comprises a HC having an amino acid sequence at least 80% identical to the amino acid sequence of SEQ ID NO:951. In some embodiments of the multispecific antibody provided herein, the second binding domain that binds CD3 comprises a HC having an amino acid sequence at least 80% identical to the amino acid sequence of SEQ ID NO:985. In some embodiments of the multispecific antibody provided herein, the second binding domain that binds CD3 comprises a HC having an amino acid sequence at least 80% identical to the amino acid sequence of SEQ ID NO:1019. In some embodiments of the multispecific antibody provided herein, the second binding domain that binds CD3 comprises a HC having an amino acid sequence at least 80% identical to the amino acid sequence of SEQ ID NO:1504. In some embodiments of the multispecific antibody provided herein, the second binding domain that binds CD3 comprises a HC having an amino acid sequence at least 80% identical to the amino acid sequence of SEQ ID NO:1455. In some embodiments of the multispecific antibody provided herein, the second binding domain that binds CD3 comprises a HC having an amino acid sequence at least 80% identical to the amino acid sequence of SEQ ID NO:1192. In some embodiments of the multispecific antibody provided herein, the second binding domain that binds CD3 comprises a HC having an amino acid sequence at least 80% identical to the amino acid sequence of SEQ ID NO:1194. In some embodiments of the multispecific antibody provided herein, the second binding domain that binds CD3 comprises a HC having an amino acid sequence at least 80% identical to the amino acid sequence of SEQ ID NO:1167. In some embodiments of the multispecific antibody provided herein, the second binding domain that binds CD3 comprises a HC having an amino acid sequence at least 80% identical to the amino acid sequence of SEQ ID NO:1218. In some embodiments of the multispecific antibody provided herein, the second binding domain that binds CD3 comprises a HC having an amino acid sequence at least 80% identical to the amino acid sequence of SEQ ID NO:1238.

In some embodiments of the multispecific antibody provided herein, the second binding domain that binds CD3 comprises a LC having an amino acid sequence at least 80% identical to the amino acid sequence of SEQ ID NO:850. In some embodiments of the multispecific antibody provided herein, the second binding domain that binds CD3 comprises a LC having an amino acid sequence at least 80% identical to the amino acid sequence of SEQ ID NO:918. In some embodiments of the multispecific antibody provided herein, the second binding domain that binds CD3 comprises a LC having an amino acid sequence at least 80% identical to the amino acid sequence of SEQ ID NO:986. In some embodiments of the multispecific antibody provided herein, the second binding domain that binds CD3 comprises a LC having an amino acid sequence at least 80% identical to the amino acid sequence of SEQ ID NO:1193. In some embodiments of the multispecific antibody provided herein, the second binding domain that binds CD3 comprises a LC having an amino acid sequence at least 80% identical to the amino acid sequence of SEQ ID NO:1195. In some embodiments of the multispecific antibody provided herein, the second binding domain that binds CD3 comprises a LC having an amino acid sequence at least 80% identical to the amino acid sequence of SEQ ID NO:1219.

In some embodiments of the multispecific PSMAxCD3 antibody provided herein, the second binding domain that binds CD3 comprises a scFv having an amino acid sequence of SEQ ID NO:1186. In some embodiments of the multispecific antibody provided herein, the second binding domain that binds CD3 comprises a scFv having an amino acid sequence of SEQ ID NO:1187. In some embodiments of the multispecific antibody provided herein, the second binding domain that binds CD3 comprises a scFv having an amino acid sequence of SEQ ID NO:1523. In some embodiments of the multispecific antibody provided herein, the second binding domain that binds CD3 comprises a scFv having an amino acid sequence of SEQ ID NO:1524. In some embodiments of the multispecific antibody provided herein, the second binding domain that binds CD3 comprises a scFv having an amino acid sequence at least 80% identical to the amino acid sequence of SEQ ID NO:1186. In some embodiments of the multispecific antibody provided herein, the second binding domain that binds CD3 comprises a scFv having an amino acid sequence at least 80% identical to the amino acid sequence of SEQ ID NO:1187. In some embodiments of the multispecific antibody provided herein, the second binding domain that binds CD3 comprises a scFv having an amino acid sequence at least 80% identical to the amino acid sequence of SEQ ID NO:1523. In some embodiments of the multispecific antibody provided herein, the second binding domain that binds CD3 comprises a scFv having an amino acid sequence at least 80% identical to the amino acid sequence of SEQ ID NO:1524.

Provided herein is an isolated bispecific antibody comprising a first binding domain that binds PSMA and a second binding domain that binds CD3, wherein the first binding domain that binds PSMA comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270, and the second binding domain that binds CD3 comprises a scFv comprising a VH CDR1, a VH CDR2, a VH CDR3, a VL CDR1, a VL CDR2, and a VL CDR3 of a scFv having an amino acid sequence of SEQ ID NO:1524. In some embodiments of the multispecific PSMAxCD3 antibody provided herein, the first binding domain that binds PSMA comprises a VH CDR1, VH CDR2, VH CDR3, VL CDR1, VL CDR2, VL CDR3 of SEQ ID NO:205, 206, 411, 242, 209 and 244, respectively, wherein the amino acid sequences are according to the Kabat numbering system; and the second binding domain that binds CD3 comprises a VH CDR1, VH CDR2, VH CDR3, VL CDR1, VL CDR2, VL CDR3 of SEQ ID NO:1467, 1468, 1506, 1470, 1471 and 1472, respectively; wherein the amino acid sequences are according to the Kabat numbering system. In some embodiments of the multispecific PSMAxCD3 antibody provided herein, the first binding domain that binds PSMA comprises (i) a VH having an amino acid sequence of SEQ ID NO:439; and (ii) a VL having an amino acid sequence of SEQ ID NO:270, and the second binding domain that binds CD3 comprises a scFv of SEQ ID NO:1524, respectively. In some embodiments of the multispecific PSMAxCD3 antibody provided herein, the first binding domain that binds PSMA comprises (i) a HC2 having an amino acid sequence of SEQ ID NO:441; and (ii) a LC2 having an amino acid sequence of SEQ ID NO:272, respectively; and the second binding domain that binds CD3 comprises a HC1 of SEQ ID NO:1455.

Provided herein is an isolated bispecific antibody comprising a first binding domain that binds PSMA and a second binding domain that binds CD3, wherein the first binding domain that binds PSMA comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270, and the second binding domain that binds CD3 comprises a scFv comprising a VH CDR1, a VH CDR2, a VH CDR3, a VL CDR1, a VL CDR2, and a VL CDR3 of a scFv having an amino acid sequence of SEQ ID NO:1286. In some embodiments of the multispecific PSMAxCD3 antibody provided herein, the first binding domain that binds PSMA comprises a VH CDR1, VH CDR2, VH CDR3, VL CDR1, VL CDR2, VL CDR3 of SEQ ID NO:205, 206, 411, 242, 209 and 244, respectively, wherein the amino acid sequences are according to the Kabat numbering system; and the second binding domain that binds CD3 comprises a VH CDR1, VH CDR2, VH CDR3, VL CDR1, VL CDR2, VL CDR3 of SEQ ID NO:1, 954, 955, 956, 957 and 958, respectively; wherein the amino acid sequences are according to the Kabat numbering system. In some embodiments of the multispecific PSMAxCD3 antibody provided herein, the first binding domain that binds PSMA comprises (i) a VH having an amino acid sequence of SEQ ID NO:439; and (ii) a VL having an amino acid sequence of SEQ ID NO:270, and the second binding domain that binds CD3 comprises a scFv of SEQ ID NO:1186, respectively. In some embodiments of the multispecific PSMAxCD3 antibody provided herein, the first binding domain that binds PSMA comprises (i) a HC2 having an amino acid sequence of SEQ ID NO:441; and (ii) a LC2 having an amino acid sequence of SEQ ID NO:272, respectively; and the second binding domain that binds CD3 comprises a HC1 of SEQ ID NO:1192.

Provided herein is an isolated bispecific antibody comprising a first binding domain that binds PSMA and a second binding domain that binds CD3, wherein the first binding domain that binds PSMA comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406, and the second binding domain that binds CD3 comprises a scFv comprising a VH CDR1, a VH CDR2, a VH CDR3, a VL CDR1, a VL CDR2, and a VL CDR3 of a scFv having an amino acid sequence of SEQ ID NO:1523. In some embodiments of the multispecific PSMAxCD3 antibody provided herein, the first binding domain that binds PSMA comprises a VH CDR1, VH CDR2, VH CDR3, VL CDR1, VL CDR2, VL CDR3 of SEQ ID NO:375, 376, 377, 378, 379 and 380, respectively, wherein the amino acid sequences are according to the Kabat numbering system; and the second binding domain that binds CD3 comprises a VH CDR1, VH CDR2, VH CDR3, VL CDR1, VL CDR2, VL CDR3 of SEQ ID NO:1467, 1468, 1469, 1470, 1471 and 1472, respectively; wherein the amino acid sequences are according to the Kabat numbering system. In some embodiments of the multispecific PSMAxCD3 antibody provided herein, the first binding domain that binds PSMA comprises (i) a VH having an amino acid sequence of SEQ ID NO:405; and (ii) a VL having an amino acid sequence of SEQ ID NO:406, and the second binding domain that binds CD3 comprises a scFv of SEQ ID NO:1523, respectively. In some embodiments of the multispecific PSMAxCD3 antibody provided herein, the first binding domain that binds PSMA comprises (i) a HC2 having an amino acid sequence of SEQ ID NO:407; and (ii) a LC2 having an amino acid sequence of SEQ ID NO:408, respectively; and the second binding domain that binds CD3 comprises a HC1 of SEQ ID NO:1504.

Provided herein is an isolated bispecific antibody comprising a first binding domain that binds PSMA and a second binding domain that binds CD3, wherein the first binding domain that binds PSMA comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270, and the second binding domain that binds CD3 comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In some embodiments of the multispecific PSMAxCD3 antibody provided herein, the first binding domain that binds PSMA comprises a VH CDR1, VH CDR2, VH CDR3, VL CDR1, VL CDR2, VL CDR3 of SEQ ID NO:205, 206, 411, 242, 209 and 244, respectively, wherein the amino acid sequences are according to the Kabat numbering system; and the second binding domain that binds CD3 comprises a VH CDR1, VH CDR2, VH CDR3, VL CDR1, VL CDR2, VL CDR3 of SEQ ID NO:817, 818, 819, 820, 821 and 822, respectively; wherein the amino acid sequences are according to the Kabat numbering system. In some embodiments of the multispecific PSMAxCD3 antibody provided herein, the first binding domain that binds PSMA comprises (i) a VH having an amino acid sequence of SEQ ID NO:439; and (ii) a VL having an amino acid sequence of SEQ ID NO:270, and the second binding domain that binds CD3 comprises (i) a VH having an amino acid sequence of SEQ ID NO:847; and (ii) a VL having an amino acid sequence of SEQ ID NO:848, respectively. In some embodiments of the multispecific PSMAxCD3 antibody provided herein, the first binding domain that binds PSMA comprises (i) a HC2 having an amino acid sequence of SEQ ID NO:1244; and (ii) a LC2 having an amino acid sequence of SEQ ID NO:272, respectively; and the second binding domain that binds CD3 comprises a (i) HC1 having an amino acid sequence of SEQ ID NO:849; and (ii) LC1 having an amino acid sequence of SEQ ID NO:850. In some embodiments of the multispecific PSMAxCD3 antibody provided herein, the first binding domain that binds PSMA comprises (i) a HC2 having an amino acid sequence of SEQ ID NO:1244; and (ii) a LC2 having an amino acid sequence of SEQ ID NO:272, respectively; and the second binding domain that binds CD3 comprises a (i) HC1 having an amino acid sequence of SEQ ID NO:883; and (ii) LC1 having an amino acid sequence of SEQ ID NO:850.

Exemplary PSMA antibody embodiments are provided herein, including in the Examples and Tables 4-12. Exemplary CD3 antibody embodiments are provided herein, including in the Examples and Tables 16-22. Exemplary PSMAxCD3 antibody embodiments are provided herein, including in the Examples and Tables 23-28.

In certain embodiments, the multispecific PSMA antibody comprises a first binding domain of PSMB889. In other embodiments, the multispecific PSMA antibody comprises a first binding domain of PSMB890. In certain embodiments, the multispecific PSMA antibody comprises a first binding domain of PSMB891. In certain embodiments, the multispecific PSMA antibody comprises a first binding domain of PSMB892. In other embodiments, the multispecific PSMA antibody comprises a first binding domain of PSMB893. In certain embodiments, the multispecific PSMA antibody comprises a first binding domain of PSMB894. In other embodiments, the multispecific PSMA antibody comprises a first binding domain of PSMB895. In certain embodiments, the multispecific PSMA antibody comprises a first binding domain of PSMB896. In certain embodiments, the multispecific PSMA antibody comprises a first binding domain of PSMB897. In other embodiments, the multispecific PSMA antibody comprises a first binding domain of PSMB898. In certain embodiments, the multispecific PSMA antibody comprises a first binding domain of PSMB899. In certain embodiments, the multispecific PSMA antibody comprises a first binding domain of PSMHB49SC1133_011A11_1. In other embodiments, the multispecific PSMA antibody comprises a first binding domain of PSMB896-G100A. In certain embodiments, the multispecific PSMA antibody comprises a first binding domain of PSMA_P72_A10-HC-G54E. In certain embodiments, the multispecific PSMA antibody comprises a first binding domain of PSMA_P72_D01-HC-D95E. In other embodiments, the multispecific PSMA antibody comprises a first binding domain of PSMA_P72_F01. In other embodiments, the multispecific PSMA antibody comprises a first binding domain of PSMA_P75_F01. In certain embodiments, the multispecific PSMA antibody comprises a first binding domain of PSMA_P72_F07. In certain embodiments, the multispecific PSMA antibody comprises a first binding domain of PSMA_P72_E07. In other embodiments, the multispecific PSMA antibody comprises a first binding domain of PSMA_P72_D01. In certain embodiments, the multispecific PSMA antibody comprises a first binding domain of PSMA_P72_C01. In other embodiments, the multispecific PSMA antibody comprises a first binding domain of PSMA_P72_A10. In certain embodiments, the multispecific PSMA antibody comprises a first binding domain of PSMA_P72_F02. In certain embodiments, the multispecific PSMA antibody comprises a first binding domain of PSMA_P70_F02. In other embodiments, the multispecific PSMA antibody comprises a first binding domain of PSMA_P72_G02. In other embodiments, the multispecific PSMA antibody comprises a first binding domain of PSMA_P72_A11. Each of these antibodies is further described in the Examples below. In a specific embodiment, the multispecific PSMA antibody is a multispecific PSMAxCD3 antibody.

In some embodiments, provided is a PSMA multispecific antibody comprising a first binding domain that comprises a VH of any of the PSMA antibodies provided in Tables 4-12 or 23-28. In some embodiments, provided is a PSMA multispecific antibody comprising a first binding domain that comprises a VL of any of the PSMA antibodies provided in Tables 4-12 or 23-28. In some embodiments, provided is a PSMA multispecific antibody comprising a first binding domain that comprises a VH and VL of any of the PSMA antibodies provided in Tables 4-12 or 23-28. In other embodiments, provided is a PSMA multispecific antibody comprising a first binding domain that comprises a VH CDR1, VH CDR3 and VH CDR3 of any of the PSMA antibodies provided in Tables 4-12 or 23-28. In other embodiments, provided is a PSMA multispecific antibody comprising a first binding domain that comprises a VL CDR1, VL CDR3 and VL CDR3 of any of the PSMA antibodies provided Tables 4-12 or 23-28. In other embodiments, provided is a PSMA multispecific antibody comprising a first binding domain that comprises a VH CDR1, VH CDR3, VH CDR3, VL CDR1, VL CDR2, and VL CDR3 of any of the PSMA antibodies provided in Tables 4-12 or 23-28. In some embodiments, the VH CDR1, VH CDR2, VH CDR3, VL CDR1, VL CDR2, and VL CDR3 amino acid sequences of the PSMA antibody are according to the Kabat numbering system. In some embodiments, the VH CDR1, VH CDR2, VH CDR3, VL CDR1, VL CDR2, and VL CDR3 amino acid sequences of the PSMA antibody are according to the Chothia numbering system. In some embodiments, the VH CDR1, VH CDR2, VH CDR3, VL CDR1, VL CDR2, and VL CDR3 amino acid sequences of the PSMA antibody are according to the AbM numbering system. In some embodiments, the VH CDR1, VH CDR2, VH CDR3, VL CDR1, VL CDR2, and VL CDR3 amino acid sequences of the PSMA antibody are according to the Contact numbering system. In some embodiments, the VH CDR1, VH CDR2, VH CDR3, VL CDR1, VL CDR2, and VL CDR3 amino acid sequences of the PSMA antibody are according to the IMGT numbering system. In some embodiments, the VH CDR1, VH CDR2, VH CDR3, VL CDR1, VL CDR2, and VL CDR3 amino acid sequences pf the PSMA antibody are according to a combination of the numbering systems provided herein.

In some embodiments, provided is a multispecific PSMAxCD3 antibody comprising a first binding domain that comprises a VH of any of the PSMA antibodies provided in Tables 4-12 or 23-28. In some embodiments, provided is a multispecific PSMAxCD3 antibody comprising a first binding domain that comprises a VL of any of the PSMA antibodies provided in Tables 4-12 or 23-28. In some embodiments, provided is a multispecific PSMAxCD3 antibody comprising a first binding domain that comprises a VH and VL of any of the PSMA antibodies provided in Tables 4-12 or 23-28. In other embodiments, provided is a multispecific PSMAxCD3 antibody comprising a first binding domain that comprises a VH CDR1, VH CDR3 and VH CDR3 of any of the PSMA antibodies provided in Tables 4-12 or 23-28. In other embodiments, provided is a multispecific PSMAxCD3 antibody comprising a first binding domain that comprises a VL CDR1, VL CDR3 and VL CDR3 of any of the PSMA antibodies provided in Tables 4-12 or 23-28. In other embodiments, provided is a multispecific PSMAxCD3 antibody comprising a first binding domain that comprises a VH CDR1, VH CDR3, VH CDR3, VL CDR1, VL CDR2, and VL CDR3 of any of the PSMA antibodies provided in Tables 4-12 or 23-28.

In some embodiments, provided is a multispecific PSMAxCD3 antibody comprising a second binding domain that comprises a VH of any of the CD3 antibodies provided in Tables 16-22 or 23-28. In some embodiments, provided is a multispecific PSMAxCD3 antibody comprising a second binding domain that comprises a VL of any of the CD3 antibodies provided in Tables 16-22 or 23-28. In some embodiments, provided is a multispecific PSMAxCD3 antibody comprising a second binding domain that comprises a VH and VL of any of the CD3 antibodies provided in Tables 16-22 or 23-28. In other embodiments, provided is a multispecific PSMAxCD3 antibody comprising a second binding domain that comprises a VH CDR1, VH CDR3 and VH CDR3 of any of the CD3 antibodies provided in Tables 16-22 or 23-28. In other embodiments, provided is a multispecific PSMAxCD3 antibody comprising a second binding domain that comprises a VL CDR1, VL CDR3 and VL CDR3 of any of the CD3 antibodies provided in Tables 16-22 or 23-28. In other embodiments, provided is a multispecific PSMAxCD3 antibody comprising a second binding domain that comprises a VH CDR1, VH CDR3, VH CDR3, VL CDR1, VL CDR2, and VL CDR3 of any of the CD3 antibodies provided in Tables 16-22 or 23-28. In some embodiments, the VH CDR1, VH CDR2, VH CDR3, VL CDR1, VL CDR2, and VL CDR3 amino acid sequences are according to the Kabat numbering system. In some embodiments, the VH CDR1, VH CDR2, VH CDR3, VL CDR1, VL CDR2, and VL CDR3 amino acid sequences are according to the Chothia numbering system. In some embodiments, the VH CDR1, VH CDR2, VH CDR3, VL CDR1, VL CDR2, and VL CDR3 amino acid sequences are according to the AbM numbering system. In some embodiments, the VH CDR1, VH CDR2, VH CDR3, VL CDR1, VL CDR2, and VL CDR3 amino acid sequences are according to the Contact numbering system. In some embodiments, the VH CDR1, VH CDR2, VH CDR3, VL CDR1, VL CDR2, and VL CDR3 amino acid sequences are according to the IMGT numbering system. In some embodiments, the VH CDR1, VH CDR2, VH CDR3, VL CDR1, VL CDR2, and VL CDR3 amino acid sequences are according to a combination of the numbering systems provided herein.

In some embodiments, the multispecific PSMAxCD3 antibody is PS3B1353, PS3B1505, PS3B1508, PS3B1917, PS3B1918, PS3B1919, PS3B1920, PS3B1921, PS3B1922, PS3B1923, PS3B1924, PS3B1925, PS3B1926, PS3B1927, PS3B1928, PSMB2908, PSMB2909, PS3B917, PS3B918, PS3B913, PS3B915, PS3B914, PS3B916, PS3B919, PS3B921, PS3B920, PS3B922, PS3B912, PS3B930, PS3B931, PS3B926, PS3B928, PS3B927, PS3B929, PS3B932, PS3B934, PS3B933, PS3B935, PS3B925, PS3B1352, PS3B1353, PS3B1354, PS3B1355, PS3B1356, PS3B1357, PS3B1358, PSMB937, PS3B1391, or PS3B1396. In one embodiment, the multispecific PSMAxCD3 antibody is PS3B1353. In one embodiment, the multispecific PSMAxCD3 antibody is PS3B1505. In one embodiment, the multispecific PSMAxCD3 antibody is PS3B1508. In one embodiment, the multispecific PSMAxCD3 antibody is PS3B1917. In one embodiment, the multispecific PSMAxCD3 antibody is PS3B1918. In one embodiment, the multispecific PSMAxCD3 antibody is PS3B1919. In one embodiment, the multispecific PSMAxCD3 antibody is PS3B1920. In one embodiment, the multispecific PSMAxCD3 antibody is PS3B1921. In one embodiment, the multispecific PSMAxCD3 antibody is PS3B1922. In one embodiment, the multispecific PSMAxCD3 antibody is PS3B1923. In one embodiment, the multispecific PSMAxCD3 antibody is PS3B1924. In one embodiment, the multispecific PSMAxCD3 antibody is PS3B1925. In one embodiment, the multispecific PSMAxCD3 antibody is PS3B1926. In one embodiment, the multispecific PSMAxCD3 antibody is PS3B1927. In one embodiment, the multispecific PSMAxCD3 antibody is PS3B1928. In one embodiment, the multispecific PSMAxCD3 antibody is PSMB2908. In one embodiment, the multispecific PSMAxCD3 antibody is PSMB2909. In one embodiment, the multispecific PSMAxCD3 antibody is PS3B917. In one embodiment, the multispecific PSMAxCD3 antibody is PS3B918. In one embodiment, the multispecific PSMAxCD3 antibody is PS3B913. In one embodiment, the multispecific PSMAxCD3 antibody is PS3B915. In one embodiment, the multispecific PSMAxCD3 antibody is PS3B914. In one embodiment, the multispecific PSMAxCD3 antibody is PS3B916. In one embodiment, the multispecific PSMAxCD3 antibody is PS3B919. In one embodiment, the multispecific PSMAxCD3 antibody is PS3B921. In one embodiment, the multispecific PSMAxCD3 antibody is PS3B920. In one embodiment, the multispecific PSMAxCD3 antibody is PS3B922. In one embodiment, the multispecific PSMAxCD3 antibody is PS3B912. In one embodiment, the multispecific PSMAxCD3 antibody is PS3B930. In one embodiment, the multispecific PSMAxCD3 antibody is PS3B931. In one embodiment, the multispecific PSMAxCD3 antibody is PS3B926. In one embodiment, the multispecific PSMAxCD3 antibody is PS3B928. In one embodiment, the multispecific PSMAxCD3 antibody is PS3B927. In one embodiment, the multispecific PSMAxCD3 antibody is PS3B929. In one embodiment, the multispecific PSMAxCD3 antibody is PS3B932. In one embodiment, the multispecific PSMAxCD3 antibody is PS3B934. In one embodiment, the multispecific PSMAxCD3 antibody is PS3B933. In one embodiment, the multispecific PSMAxCD3 antibody is PS3B935. In one embodiment, the multispecific PSMAxCD3 antibody is PS3B925. In one embodiment, the multispecific PSMAxCD3 antibody is PS3B1352. In one embodiment, the multispecific PSMAxCD3 antibody is PS3B1353. In one embodiment, the multispecific PSMAxCD3 antibody is PS3B1354. In one embodiment, the multispecific PSMAxCD3 antibody is PS3B1355. In one embodiment, the multispecific PSMAxCD3 antibody is PS3B1356. In one embodiment, the multispecific PSMAxCD3 antibody is PS3B1357. In one embodiment, the multispecific PSMAxCD3 antibody is PS3B1358. In one embodiment, the multispecific PSMAxCD3 antibody is PSMB937. In one embodiment, the multispecific PSMAxCD3 antibody is PS3B1391. In one embodiment, the multispecific PSMAxCD3 antibody is PS3B1396.

In some embodiments, the multispecific PSMAxCD3 antibody comprises the amino acid sequence of the HC1 of PS3B1353 that binds to CD3. In some embodiments, the multispecific PSMAxCD3 antibody comprises the amino acid sequence of the LC1 of PS3B1353 that binds to CD3. In some embodiments, the multispecific PSMAxCD3 antibody comprises the amino acid sequence of the HC1 and LC1 of PS3B1353 that binds to CD3. In some embodiments, the multispecific PSMAxCD3 antibody comprises the amino acid sequence of the HC2 of PS3B1353 that binds to PSMA. In some embodiments, the multispecific PSMAxCD3 antibody comprises the amino acid sequence of the LC2 of PS3B1353 that binds to PSMA. In some embodiments, the multispecific PSMAxCD3 antibody comprises the amino acid sequence of the HC2 and LC2 of PS3B1353 that binds to PSMA.

In some embodiments, the multispecific PSMAxCD3 antibody comprises the amino acid sequence of the HC1 of PS3B1505 that binds to CD3. In some embodiments, the multispecific PSMAxCD3 antibody comprises the amino acid sequence of the LC1 of PS3B1505 that binds to CD3. In some embodiments, the multispecific PSMAxCD3 antibody comprises the amino acid sequence of the HC1 and LC1 of PS3B1505 that binds to CD3. In some embodiments, the multispecific PSMAxCD3 antibody comprises the amino acid sequence of the HC2 of PS3B1505 that binds to PSMA. In some embodiments, the multispecific PSMAxCD3 antibody comprises the amino acid sequence of the LC2 of PS3B1505 that binds to PSMA. In some embodiments, the multispecific PSMAxCD3 antibody comprises the amino acid sequence of the HC2 and LC2 of PS3B1505 that binds to PSMA.

In some embodiments, the multispecific PSMAxCD3 antibody comprises the amino acid sequence of the HC1 of PS3B1508 that binds to CD3. In some embodiments, the multispecific PSMAxCD3 antibody comprises the amino acid sequence of the HC2 of PS3B1508 that binds to PSMA. In some embodiments, the multispecific PSMAxCD3 antibody comprises the amino acid sequence of the LC2 of PS3B1508 that binds to PSMA. In some embodiments, the multispecific PSMAxCD3 antibody comprises the amino acid sequence of the HC2 and LC2 of PS3B1508 that binds to PSMA.

In some embodiments, the multispecific PSMAxCD3 antibody comprises the amino acid sequence of the HC1 of PS3B1917 that binds to CD3. In some embodiments, the multispecific PSMAxCD3 antibody comprises the amino acid sequence of the LC1 of PS3B1917 that binds to CD3. In some embodiments, the multispecific PSMAxCD3 antibody comprises the amino acid sequence of the HC1 and LC1 of PS3B1917 that binds to CD3. In some embodiments, the multispecific PSMAxCD3 antibody comprises the amino acid sequence of the HC2 of PS3B1917 that binds to PSMA.

In some embodiments, the multispecific PSMAxCD3 antibody comprises the amino acid sequence of the HC1 of PS3B1918 that binds to CD3. In some embodiments, the multispecific PSMAxCD3 antibody comprises the amino acid sequence of the LC1 of PS3B1918 that binds to CD3. In some embodiments, the multispecific PSMAxCD3 antibody comprises the amino acid sequence of the HC1 and LC1 of PS3B1918 that binds to CD3. In some embodiments, the multispecific PSMAxCD3 antibody comprises the amino acid sequence of the HC2 of PS3B1918 that binds to PSMA.

In some embodiments, the multispecific PSMAxCD3 antibody comprises the amino acid sequence of the HC1 of PS3B1919 that binds to CD3. In some embodiments, the multispecific PSMAxCD3 antibody comprises the amino acid sequence of the LC1 of PS3B1919 that binds to CD3. In some embodiments, the multispecific PSMAxCD3 antibody comprises the amino acid sequence of the HC1 and LC1 of PS3B1919 that binds to CD3. In some embodiments, the multispecific PSMAxCD3 antibody comprises the amino acid sequence of the HC2 of PS3B1919 that binds to PSMA.

In some embodiments, the multispecific PSMAxCD3 antibody comprises the amino acid sequence of the HC1 of PS3B1920 that binds to CD3. In some embodiments, the multispecific PSMAxCD3 antibody comprises the amino acid sequence of the LC1 of PS3B1920 that binds to CD3. In some embodiments, the multispecific PSMAxCD3 antibody comprises the amino acid sequence of the HC1 and LC1 of PS3B1920 that binds to CD3. In some embodiments, the multispecific PSMAxCD3 antibody comprises the amino acid sequence of the HC2 of PS3B1920 that binds to PSMA.

In some embodiments, the multispecific PSMAxCD3 antibody comprises the amino acid sequence of the HC1 of PS3B1921 that binds to CD3. In some embodiments, the multispecific PSMAxCD3 antibody comprises the amino acid sequence of the LC1 of PS3B1921 that binds to CD3. In some embodiments, the multispecific PSMAxCD3 antibody comprises the amino acid sequence of the HC1 and LC1 of PS3B1921 that binds to CD3. In some embodiments, the multispecific PSMAxCD3 antibody comprises the amino acid sequence of the HC2 of PS3B1921 that binds to PSMA.

In some embodiments, the multispecific PSMAxCD3 antibody comprises the amino acid sequence of the HC1 of PS3B1922 that binds to CD3. In some embodiments, the multispecific PSMAxCD3 antibody comprises the amino acid sequence of the LC1 of PS3B1922 that binds to CD3. In some embodiments, the multispecific PSMAxCD3 antibody comprises the amino acid sequence of the HC1 and LC1 of PS3B1922 that binds to CD3. In some embodiments, the multispecific PSMAxCD3 antibody comprises the amino acid sequence of the HC2 of PS3B1922 that binds to PSMA.

In some embodiments, the multispecific PSMAxCD3 antibody comprises the amino acid sequence of the HC1 of PS3B1923 that binds to CD3. In some embodiments, the multispecific PSMAxCD3 antibody comprises the amino acid sequence of the LC1 of PS3B1923 that binds to CD3. In some embodiments, the multispecific PSMAxCD3 antibody comprises the amino acid sequence of the HC1 and LC1 of PS3B1923 that binds to CD3. In some embodiments, the multispecific PSMAxCD3 antibody comprises the amino acid sequence of the HC2 of PS3B1923 that binds to PSMA.

In some embodiments, the multispecific PSMAxCD3 antibody comprises the amino acid sequence of the HC1 of PS3B1924 that binds to CD3. In some embodiments, the multispecific PSMAxCD3 antibody comprises the amino acid sequence of the LC1 of PS3B1924 that binds to CD3. In some embodiments, the multispecific PSMAxCD3 antibody comprises the amino acid sequence of the HC1 and LC1 of PS3B1924 that binds to CD3. In some embodiments, the multispecific PSMAxCD3 antibody comprises the amino acid sequence of the HC2 of PS3B1924 that binds to PSMA.

In some embodiments, the multispecific PSMAxCD3 antibody comprises the amino acid sequence of the HC1 of PS3B1925 that binds to CD3. In some embodiments, the multispecific PSMAxCD3 antibody comprises the amino acid sequence of the LC1 of PS3B1925 that binds to CD3. In some embodiments, the multispecific PSMAxCD3 antibody comprises the amino acid sequence of the HC1 and LC1 of PS3B1925 that binds to CD3. In some embodiments, the multispecific PSMAxCD3 antibody comprises the amino acid sequence of the HC2 of PS3B1925 that binds to PSMA.

In some embodiments, the multispecific PSMAxCD3 antibody comprises the amino acid sequence of the HC1 of PS3B1926 that binds to CD3. In some embodiments, the multispecific PSMAxCD3 antibody comprises the amino acid sequence of the LC1 of PS3B1926 that binds to CD3. In some embodiments, the multispecific PSMAxCD3 antibody comprises the amino acid sequence of the HC1 and LC1 of PS3B1926 that binds to CD3. In some embodiments, the multispecific PSMAxCD3 antibody comprises the amino acid sequence of the HC2 of PS3B1926 that binds to PSMA.

In some embodiments, the multispecific PSMAxCD3 antibody comprises the amino acid sequence of the HC1 of PS3B1927 that binds to CD3. In some embodiments, the multispecific PSMAxCD3 antibody comprises the amino acid sequence of the LC1 of PS3B1927 that binds to CD3. In some embodiments, the multispecific PSMAxCD3 antibody comprises the amino acid sequence of the HC1 and LC1 of PS3B1927 that binds to CD3. In some embodiments, the multispecific PSMAxCD3 antibody comprises the amino acid sequence of the HC2 of PS3B1927 that binds to PSMA.

In some embodiments, the multispecific PSMAxCD3 antibody comprises the amino acid sequence of the HC1 of PS3B1928 that binds to CD3. In some embodiments, the multispecific PSMAxCD3 antibody comprises the amino acid sequence of the LC1 of PS3B1928 that binds to CD3. In some embodiments, the multispecific PSMAxCD3 antibody comprises the amino acid sequence of the HC1 and LC1 of PS3B1928 that binds to CD3. In some embodiments, the multispecific PSMAxCD3 antibody comprises the amino acid sequence of the HC2 of PS3B1928 that binds to PSMA.

In some embodiments, the multispecific PSMAxCD3 antibody comprises the amino acid sequence of the HC1 of PS3B917 that binds to CD3. In some embodiments, the multispecific PSMAxCD3 antibody comprises the amino acid sequence of the LC1 of PS3B917 that binds to CD3. In some embodiments, the multispecific PSMAxCD3 antibody comprises the amino acid sequence of the HC1 and LC1 of PS3B917 that binds to CD3. In some embodiments, the multispecific PSMAxCD3 antibody comprises the amino acid sequence of the HC2 of PS3B917 that binds to PSMA. In some embodiments, the multispecific PSMAxCD3 antibody comprises the amino acid sequence of the LC2 of PS3B917 that binds to PSMA. In some embodiments, the multispecific PSMAxCD3 antibody comprises the amino acid sequence of the HC2 and LC2 of PS3B917 that binds to PSMA.

In some embodiments, the multispecific PSMAxCD3 antibody comprises the amino acid sequence of the HC1 of PS3B918 that binds to CD3. In some embodiments, the multispecific PSMAxCD3 antibody comprises the amino acid sequence of the LC1 of PS3B918 that binds to CD3. In some embodiments, the multispecific PSMAxCD3 antibody comprises the amino acid sequence of the HC1 and LC1 of PS3B918 that binds to CD3. In some embodiments, the multispecific PSMAxCD3 antibody comprises the amino acid sequence of the HC2 of PS3B918 that binds to PSMA. In some embodiments, the multispecific PSMAxCD3 antibody comprises the amino acid sequence of the LC2 of PS3B918 that binds to PSMA. In some embodiments, the multispecific PSMAxCD3 antibody comprises the amino acid sequence of the HC2 and LC2 of PS3B918 that binds to PSMA.

In some embodiments, the multispecific PSMAxCD3 antibody comprises the amino acid sequence of the HC1 of PS3B913 that binds to CD3. In some embodiments, the multispecific PSMAxCD3 antibody comprises the amino acid sequence of the LC1 of PS3B913 that binds to CD3. In some embodiments, the multispecific PSMAxCD3 antibody comprises the amino acid sequence of the HC1 and LC1 of PS3B913 that binds to CD3. In some embodiments, the multispecific PSMAxCD3 antibody comprises the amino acid sequence of the HC2 of PS3B913 that binds to PSMA. In some embodiments, the PS3B913 that binds to PSMA. In some embodiments, the multispecific PSMAxCD3 antibody comprises the amino acid sequence of the HC2 and LC2 of PS3B913 that binds to PSMA.

In some embodiments, the multispecific PSMAxCD3 antibody comprises the amino acid sequence of the HC1 of PS3B915 that binds to CD3. In some embodiments, the multispecific PSMAxCD3 antibody comprises the amino acid sequence of the LC1 of PS3B915 that binds to CD3. In some embodiments, the multispecific PSMAxCD3 antibody comprises the amino acid sequence of the HC1 and LC1 of PS3B915 that binds to CD3. In some embodiments, the multispecific PSMAxCD3 antibody comprises the amino acid sequence of the HC2 of PS3B915 that binds to PSMA. In some embodiments, the multispecific PSMAxCD3 antibody comprises the amino acid sequence of the LC2 of PS3B915 that binds to PSMA. In some embodiments, the multispecific PSMAxCD3 antibody comprises the amino acid sequence of the HC2 and LC2 of PS3B915 that binds to PSMA.

In some embodiments, the multispecific PSMAxCD3 antibody comprises the amino acid sequence of the HC1 of PS3B914 that binds to CD3. In some embodiments, the multispecific PSMAxCD3 antibody comprises the amino acid sequence of the LC1 of PS3B914 that binds to CD3. In some embodiments, the multispecific PSMAxCD3 antibody comprises the amino acid sequence of the HC1 and LC1 of PS3B914 that binds to CD3. In some embodiments, the multispecific PSMAxCD3 antibody comprises the amino acid sequence of the HC2 of PS3B914 that binds to PSMA. In some embodiments, the multispecific PSMAxCD3 antibody comprises the amino acid sequence of the LC2 of PS3B914 that binds to PSMA. In some embodiments, the multispecific PSMAxCD3 antibody comprises the amino acid sequence of the HC2 and LC2 of PS3B914 that binds to PSMA.

In some embodiments, the multispecific PSMAxCD3 antibody comprises the amino acid sequence of the HC1 of PS3B916 that binds to CD3. In some embodiments, the multispecific PSMAxCD3 antibody comprises the amino acid sequence of the LC1 of PS3B916 that binds to CD3. In some embodiments, the multispecific PSMAxCD3 antibody comprises the amino acid sequence of the HC1 and LC1 of PS3B916 that binds to CD3. In some embodiments, the multispecific PSMAxCD3 antibody comprises the amino acid sequence of the HC2 of PS3B916 that binds to PSMA. In some embodiments, the PS3B916 that binds to PSMA. In some embodiments, the multispecific PSMAxCD3 antibody comprises the amino acid sequence of the HC2 and LC2 of PS3B916 that binds to PSMA.

In some embodiments, the multispecific PSMAxCD3 antibody comprises the amino acid sequence of the HC1 of PS3B919 that binds to CD3. In some embodiments, the multispecific PSMAxCD3 antibody comprises the amino acid sequence of the LC1 of PS3B919 that binds to CD3. In some embodiments, the multispecific PSMAxCD3 antibody comprises the amino acid sequence of the HC1 and LC1 of PS3B919 that binds to CD3. In some embodiments, the multispecific PSMAxCD3 antibody comprises the amino acid sequence of the HC2 of PS3B919 that binds to PSMA. In some embodiments, the multispecific PSMAxCD3 antibody comprises the amino acid sequence of the LC2 of PS3B919 that binds to PSMA. In some embodiments, the multispecific PSMAxCD3 antibody comprises the amino acid sequence of the HC2 and LC2 of PS3B919 that binds to PSMA.

In some embodiments, the multispecific PSMAxCD3 antibody comprises the amino acid sequence of the HC1 of PS3B921 that binds to CD3. In some embodiments, the multispecific PSMAxCD3 antibody comprises the amino acid sequence of the LC1 of PS3B921 that binds to CD3. In some embodiments, the multispecific PSMAxCD3 antibody comprises the amino acid sequence of the HC1 and LC1 of PS3B921 that binds to CD3. In some embodiments, the multispecific PSMAxCD3 antibody comprises the amino acid sequence of the HC2 of PS3B921 that binds to PSMA. In some embodiments, the multispecific PSMAxCD3 antibody comprises the amino acid sequence of the LC2 of PS3B921 that binds to PSMA. In some embodiments, the multispecific PSMAxCD3 antibody comprises the amino acid sequence of the HC2 and LC2 of PS3B921 that binds to PSMA.

In some embodiments, the multispecific PSMAxCD3 antibody comprises the amino acid sequence of the HC1 of PS3B920 that binds to CD3. In some embodiments, the multispecific PSMAxCD3 antibody comprises the amino acid sequence of the LC1 of PS3B920 that binds to CD3. In some embodiments, the multispecific PSMAxCD3 antibody comprises the amino acid sequence of the HC1 and LC1 of PS3B920 that binds to CD3. In some embodiments, the multispecific PSMAxCD3 antibody comprises the amino acid sequence of the HC2 of PS3B920 that binds to PSMA. In some embodiments, the PS3B920 that binds to PSMA. In some embodiments, the multispecific PSMAxCD3 antibody comprises the amino acid sequence of the HC2 and LC2 of PS3B920 that binds to PSMA.

In some embodiments, the multispecific PSMAxCD3 antibody comprises the amino acid sequence of the HC1 of PS3B922 that binds to CD3. In some embodiments, the multispecific PSMAxCD3 antibody comprises the amino acid sequence of the LC1 of PS3B922 that binds to CD3. In some embodiments, the multispecific PSMAxCD3 antibody comprises the amino acid sequence of the HC1 and LC1 of PS3B922 that binds to CD3. In some embodiments, the multispecific PSMAxCD3 antibody comprises the amino acid sequence of the HC2 of PS3B922 that binds to PSMA. In some embodiments, the multispecific PSMAxCD3 antibody comprises the amino acid sequence of the LC2 of PS3B922 that binds to PSMA. In some embodiments, the multispecific PSMAxCD3 antibody comprises the amino acid sequence of the HC2 and LC2 of PS3B922 that binds to PSMA.

In some embodiments, the multispecific PSMAxCD3 antibody comprises the amino acid sequence of the HC1 of PS3B912 that binds to CD3. In some embodiments, the multispecific PSMAxCD3 antibody comprises the amino acid sequence of the LC1 of PS3B912 that binds to CD3. In some embodiments, the multispecific PSMAxCD3 antibody comprises the amino acid sequence of the HC1 and LC1 of PS3B912 that binds to CD3. In some embodiments, the multispecific PSMAxCD3 antibody comprises the amino acid sequence of the HC2 of PS3B912 that binds to PSMA. In some embodiments, the multispecific PSMAxCD3 antibody comprises the amino acid sequence of the LC2 of PS3B912 that binds to PSMA. In some embodiments, the multispecific PSMAxCD3 antibody comprises the amino acid sequence of the HC2 and LC2 of PS3B912 that binds to PSMA.

In some embodiments, the multispecific PSMAxCD3 antibody comprises the amino acid sequence of the HC1 of PS3B930 that binds to CD3. In some embodiments, the multispecific PSMAxCD3 antibody comprises the amino acid sequence of the LC1 of PS3B930 that binds to CD3. In some embodiments, the multispecific PSMAxCD3 antibody comprises the amino acid sequence of the HC1 and LC1 of PS3B930 that binds to CD3. In some embodiments, the multispecific PSMAxCD3 antibody comprises the amino acid sequence of the HC2 of PS3B930 that binds to PSMA. In some embodiments, the PS3B930 that binds to PSMA. In some embodiments, the multispecific PSMAxCD3 antibody comprises the amino acid sequence of the HC2 and LC2 of PS3B930 that binds to PSMA.

In some embodiments, the multispecific PSMAxCD3 antibody comprises the amino acid sequence of the HC1 of PS3B931 that binds to CD3. In some embodiments, the multispecific PSMAxCD3 antibody comprises the amino acid sequence of the LC1 of PS3B931 that binds to CD3. In some embodiments, the multispecific PSMAxCD3 antibody comprises the amino acid sequence of the HC1 and LC1 of PS3B931 that binds to CD3. In some embodiments, the multispecific PSMAxCD3 antibody comprises the amino acid sequence of the HC2 of PS3B931 that binds to PSMA. In some embodiments, the multispecific PSMAxCD3 antibody comprises the amino acid sequence of the LC2 of PS3B931 that binds to PSMA. In some embodiments, the multispecific PSMAxCD3 antibody comprises the amino acid sequence of the HC2 and LC2 of PS3B931 that binds to PSMA.

In some embodiments, the multispecific PSMAxCD3 antibody comprises the amino acid sequence of the HC1 of PS3B926 that binds to CD3. In some embodiments, the multispecific PSMAxCD3 antibody comprises the amino acid sequence of the LC1 of PS3B926 that binds to CD3. In some embodiments, the multispecific PSMAxCD3 antibody comprises the amino acid sequence of the HC1 and LC1 of PS3B926 that binds to CD3. In some embodiments, the multispecific PSMAxCD3 antibody comprises the amino acid sequence of the HC2 of PS3B926 that binds to PSMA. In some embodiments, the multispecific PSMAxCD3 antibody comprises the amino acid sequence of the LC2 of PS3B926 that binds to PSMA. In some embodiments, the multispecific PSMAxCD3 antibody comprises the amino acid sequence of the HC2 and LC2 of PS3B926 that binds to PSMA.

In some embodiments, the multispecific PSMAxCD3 antibody comprises the amino acid sequence of the HC1 of PS3B928 that binds to CD3. In some embodiments, the multispecific PSMAxCD3 antibody comprises the amino acid sequence of the LC1 of PS3B928 that binds to CD3. In some embodiments, the multispecific PSMAxCD3 antibody comprises the amino acid sequence of the HC1 and LC1 of PS3B928 that binds to CD3. In some embodiments, the multispecific PSMAxCD3 antibody comprises the amino acid sequence of the HC2 of PS3B928 that binds to PSMA. In some embodiments, the PS3B928 that binds to PSMA. In some embodiments, the multispecific PSMAxCD3 antibody comprises the amino acid sequence of the HC2 and LC2 of PS3B928 that binds to PSMA.

In some embodiments, the multispecific PSMAxCD3 antibody comprises the amino acid sequence of the HC1 of PS3B927 that binds to CD3. In some embodiments, the multispecific PSMAxCD3 antibody comprises the amino acid sequence of the LC1 of PS3B927 that binds to CD3. In some embodiments, the multispecific PSMAxCD3 antibody comprises the amino acid sequence of the HC1 and LC1 of PS3B927 that binds to CD3. In some embodiments, the multispecific PSMAxCD3 antibody comprises the amino acid sequence of the HC2 of PS3B927 that binds to PSMA. In some embodiments, the multispecific PSMAxCD3 antibody comprises the amino acid sequence of the LC2 of PS3B927 that binds to PSMA. In some embodiments, the multispecific PSMAxCD3 antibody comprises the amino acid sequence of the HC2 and LC2 of PS3B927 that binds to PSMA.

In some embodiments, the multispecific PSMAxCD3 antibody comprises the amino acid sequence of the HC1 of PS3B929 that binds to CD3. In some embodiments, the multispecific PSMAxCD3 antibody comprises the amino acid sequence of the LC1 of PS3B929 that binds to CD3. In some embodiments, the multispecific PSMAxCD3 antibody comprises the amino acid sequence of the HC1 and LC1 of PS3B929 that binds to CD3. In some embodiments, the multispecific PSMAxCD3 antibody comprises the amino acid sequence of the HC2 of PS3B929 that binds to PSMA. In some embodiments, the multispecific PSMAxCD3 antibody comprises the amino acid sequence of the LC2 of PS3B929 that binds to PSMA. In some embodiments, the multispecific PSMAxCD3 antibody comprises the amino acid sequence of the HC2 and LC2 of PS3B929 that binds to PSMA.

In some embodiments, the multispecific PSMAxCD3 antibody comprises the amino acid sequence of the HC1 of PS3B932 that binds to CD3. In some embodiments, the multispecific PSMAxCD3 antibody comprises the amino acid sequence of the LC1 of PS3B932 that binds to CD3. In some embodiments, the multispecific PSMAxCD3 antibody comprises the amino acid sequence of the HC1 and LC1 of PS3B932 that binds to CD3. In some embodiments, the multispecific PSMAxCD3 antibody comprises the amino acid sequence of the HC2 of PS3B932 that binds to PSMA. In some embodiments, the PS3B932 that binds to PSMA. In some embodiments, the multispecific PSMAxCD3 antibody comprises the amino acid sequence of the HC2 and LC2 of PS3B932 that binds to PSMA.

In some embodiments, the multispecific PSMAxCD3 antibody comprises the amino acid sequence of the HC1 of PS3B934 that binds to CD3. In some embodiments, the multispecific PSMAxCD3 antibody comprises the amino acid sequence of the LC1 of PS3B934 that binds to CD3. In some embodiments, the multispecific PSMAxCD3 antibody comprises the amino acid sequence of the HC1 and LC1 of PS3B934 that binds to CD3. In some embodiments, the multispecific PSMAxCD3 antibody comprises the amino acid sequence of the HC2 of PS3B934 that binds to PSMA. In some embodiments, the multispecific PSMAxCD3 antibody comprises the amino acid sequence of the LC2 of PS3B934 that binds to PSMA. In some embodiments, the multispecific PSMAxCD3 antibody comprises the amino acid sequence of the HC2 and LC2 of PS3B934 that binds to PSMA.

In some embodiments, the multispecific PSMAxCD3 antibody comprises the amino acid sequence of the HC1 of PS3B933 that binds to CD3. In some embodiments, the multispecific PSMAxCD3 antibody comprises the amino acid sequence of the LC1 of PS3B933 that binds to CD3. In some embodiments, the multispecific PSMAxCD3 antibody comprises the amino acid sequence of the HC1 and LC1 of PS3B933 that binds to CD3. In some embodiments, the multispecific PSMAxCD3 antibody comprises the amino acid sequence of the HC2 of PS3B933 that binds to PSMA. In some embodiments, the multispecific PSMAxCD3 antibody comprises the amino acid sequence of the LC2 of PS3B933 that binds to PSMA. In some embodiments, the multispecific PSMAxCD3 antibody comprises the amino acid sequence of the HC2 and LC2 of PS3B933 that binds to PSMA.

In some embodiments, the multispecific PSMAxCD3 antibody comprises the amino acid sequence of the HC1 of PS3B935 that binds to CD3. In some embodiments, the multispecific PSMAxCD3 antibody comprises the amino acid sequence of the LC1 of PS3B935 that binds to CD3. In some embodiments, the multispecific PSMAxCD3 antibody comprises the amino acid sequence of the HC1 and LC1 of PS3B935 that binds to CD3. In some embodiments, the multispecific PSMAxCD3 antibody comprises the amino acid sequence of the HC2 of PS3B935 that binds to PSMA. In some embodiments, the PS3B935 that binds to PSMA. In some embodiments, the multispecific PSMAxCD3 antibody comprises the amino acid sequence of the HC2 and LC2 of PS3B935 that binds to PSMA.

In some embodiments, the multispecific PSMAxCD3 antibody comprises the amino acid sequence of the HC1 of PS3B925 that binds to CD3. In some embodiments, the multispecific PSMAxCD3 antibody comprises the amino acid sequence of the LC1 of PS3B925 that binds to CD3. In some embodiments, the multispecific PSMAxCD3 antibody comprises the amino acid sequence of the HC1 and LC1 of PS3B925 that binds to CD3. In some embodiments, the multispecific PSMAxCD3 antibody comprises the amino acid sequence of the HC2 of PS3B925 that binds to PSMA. In some embodiments, the multispecific PSMAxCD3 antibody comprises the amino acid sequence of the LC2 of PS3B925 that binds to PSMA. In some embodiments, the multispecific PSMAxCD3 antibody comprises the amino acid sequence of the HC2 and LC2 of PS3B925 that binds to PSMA.

In some embodiments, the multispecific PSMAxCD3 antibody comprises the amino acid sequence of the HC1 of PS3B1352 that binds to CD3. In some embodiments, the multispecific PSMAxCD3 antibody comprises the amino acid sequence of the LC1 of PS3B1352 that binds to CD3. In some embodiments, the multispecific PSMAxCD3 antibody comprises the amino acid sequence of the HC1 and LC1 of PS3B1352 that binds to CD3. In some embodiments, the multispecific PSMAxCD3 antibody comprises the amino acid sequence of the HC2 of PS3B1352 that binds to PSMA. In some embodiments, the multispecific PSMAxCD3 antibody comprises the amino acid sequence of the LC2 of PS3B1352 that binds to PSMA. In some embodiments, the multispecific PSMAxCD3 antibody comprises the amino acid sequence of the HC2 and LC2 of PS3B1352 that binds to PSMA.

In some embodiments, the multispecific PSMAxCD3 antibody comprises the amino acid sequence of the HC1 of PS3B1353 that binds to CD3. In some embodiments, the multispecific PSMAxCD3 antibody comprises the amino acid sequence of the LC1 of PS3B1353 that binds to CD3. In some embodiments, the multispecific PSMAxCD3 antibody comprises the amino acid sequence of the HC1 and LC1 of PS3B1353 that binds to CD3. In some embodiments, the multispecific PSMAxCD3 antibody comprises the amino acid sequence of the HC2 of PS3B1353 that binds to PSMA. In some embodiments, the PS3B1353 that binds to PSMA. In some embodiments, the multispecific PSMAxCD3 antibody comprises the amino acid sequence of the HC2 and LC2 of PS3B1353 that binds to PSMA.

In some embodiments, the multispecific PSMAxCD3 antibody comprises the amino acid sequence of the HC1 of PS3B1354 that binds to CD3. In some embodiments, the multispecific PSMAxCD3 antibody comprises the amino acid sequence of the LC1 of PS3B1354 that binds to CD3. In some embodiments, the multispecific PSMAxCD3 antibody comprises the amino acid sequence of the HC1 and LC1 of PS3B1354 that binds to CD3. In some embodiments, the multispecific PSMAxCD3 antibody comprises the amino acid sequence of the HC2 of PS3B1354 that binds to PSMA. In some embodiments, the multispecific PSMAxCD3 antibody comprises the amino acid sequence of the LC2 of PS3B1354 that binds to PSMA. In some embodiments, the multispecific PSMAxCD3 antibody comprises the amino acid sequence of the HC2 and LC2 of PS3B1354 that binds to PSMA.

In some embodiments, the multispecific PSMAxCD3 antibody comprises the amino acid sequence of the HC1 of PS3B1355 that binds to CD3. In some embodiments, the multispecific PSMAxCD3 antibody comprises the amino acid sequence of the LC1 of PS3B1355 that binds to CD3. In some embodiments, the multispecific PSMAxCD3 antibody comprises the amino acid sequence of the HC1 and LC1 of PS3B1355 that binds to CD3. In some embodiments, the multispecific PSMAxCD3 antibody comprises the amino acid sequence of the HC2 of PS3B1355 that binds to PSMA. In some embodiments, the multispecific PSMAxCD3 antibody comprises the amino acid sequence of the LC2 of PS3B1355 that binds to PSMA. In some embodiments, the multispecific PSMAxCD3 antibody comprises the amino acid sequence of the HC2 and LC2 of PS3B1355 that binds to PSMA.

In some embodiments, the multispecific PSMAxCD3 antibody comprises the amino acid sequence of the HC1 of PS3B1356 that binds to CD3. In some embodiments, the multispecific PSMAxCD3 antibody comprises the amino acid sequence of the LC1 of PS3B1356 that binds to CD3. In some embodiments, the multispecific PSMAxCD3 antibody comprises the amino acid sequence of the HC1 and LC1 of PS3B1356 that binds to CD3. In some embodiments, the multispecific PSMAxCD3 antibody comprises the amino acid sequence of the HC2 of PS3B1356 that binds to PSMA. In some embodiments, the PS3B1356 that binds to PSMA. In some embodiments, the multispecific PSMAxCD3 antibody comprises the amino acid sequence of the HC2 and LC2 of PS3B1356 that binds to PSMA.

In some embodiments, the multispecific PSMAxCD3 antibody comprises the amino acid sequence of the HC1 of PS3B1357 that binds to CD3. In some embodiments, the multispecific PSMAxCD3 antibody comprises the amino acid sequence of the LC1 of PS3B1357 that binds to CD3. In some embodiments, the multispecific PSMAxCD3 antibody comprises the amino acid sequence of the HC1 and LC1 of PS3B1357 that binds to CD3. In some embodiments, the multispecific PSMAxCD3 antibody comprises the amino acid sequence of the HC2 of PS3B1357 that binds to PSMA. In some embodiments, the multispecific PSMAxCD3 antibody comprises the amino acid sequence of the LC2 of PS3B1357 that binds to PSMA. In some embodiments, the multispecific PSMAxCD3 antibody comprises the amino acid sequence of the HC2 and LC2 of PS3B1357 that binds to PSMA.

In some embodiments, the multispecific PSMAxCD3 antibody comprises the amino acid sequence of the HC1 of PS3B1358 that binds to CD3. In some embodiments, the multispecific PSMAxCD3 antibody comprises the amino acid sequence of the LC1 of PS3B1358 that binds to CD3. In some embodiments, the multispecific PSMAxCD3 antibody comprises the amino acid sequence of the HC1 and LC1 of PS3B1358 that binds to CD3. In some embodiments, the multispecific PSMAxCD3 antibody comprises the amino acid sequence of the HC2 of PS3B1358 that binds to PSMA. In some embodiments, the multispecific PSMAxCD3 antibody comprises the amino acid sequence of the LC2 of PS3B1358 that binds to PSMA. In some embodiments, the multispecific PSMAxCD3 antibody comprises the amino acid sequence of the HC2 and LC2 of PS3B1358 that binds to PSMA.

In some embodiments, the multispecific PSMAxCD3 antibody comprises the amino acid sequence of the HC1 of PS3B937 that binds to CD3. In some embodiments, the multispecific PSMAxCD3 antibody comprises the amino acid sequence of the LC1 of PS3B937 that binds to CD3. In some embodiments, the multispecific PSMAxCD3 antibody comprises the amino acid sequence of the HC1 and LC1 of PS3B937 that binds to CD3. In some embodiments, the multispecific PSMAxCD3 antibody comprises the amino acid sequence of the HC2 of PS3B937 that binds to PSMA. In some embodiments, the PS3B937 that binds to PSMA. In some embodiments, the multispecific PSMAxCD3 antibody comprises the amino acid sequence of the HC2 and LC2 of PS3B937 that binds to PSMA.

In some embodiments, the multispecific PSMAxCD3 antibody comprises the amino acid sequence of the HC1 of PS3B1391 that binds to CD3. In some embodiments, the multispecific PSMAxCD3 antibody comprises the amino acid sequence of the LC1 of PS3B1391 that binds to CD3. In some embodiments, the multispecific PSMAxCD3 antibody comprises the amino acid sequence of the HC1 and LC1 of PS3B1391 that binds to CD3. In some embodiments, the multispecific PSMAxCD3 antibody comprises the amino acid sequence of the HC2 of PS3B1391 that binds to PSMA. In some embodiments, the multispecific PSMAxCD3 antibody comprises the amino acid sequence of the LC2 of PS3B1391 that binds to PSMA. In some embodiments, the multispecific PSMAxCD3 antibody comprises the amino acid sequence of the HC2 and LC2 of PS3B1391 that binds to PSMA.

In some embodiments, the multispecific PSMAxCD3 antibody comprises the amino acid sequence of the HC1 of PS3B1396 that binds to CD3. In some embodiments, the multispecific PSMAxCD3 antibody comprises the amino acid sequence of the LC1 of PS3B1396 that binds to CD3. In some embodiments, the multispecific PSMAxCD3 antibody comprises the amino acid sequence of the HC1 and LC1 of PS3B1396 that binds to CD3. In some embodiments, the multispecific PSMAxCD3 antibody comprises the amino acid sequence of the HC2 of PS3B1396 that binds to PSMA. In some embodiments, the multispecific PSMAxCD3 antibody comprises the amino acid sequence of the LC2 of PS3B1396 that binds to PSMA. In some embodiments, the multispecific PSMAxCD3 antibody comprises the amino acid sequence of the HC2 and LC2 of PS3B1396 that binds to PSMA.

In specific embodiments, provided is a multispecific antibody comprising a PSMA antibody provided herein in a knob-in-hole format. In specific embodiments, provided is a bispecific antibody comprising a PSMA antibody provided herein in a knob-in-hole format. In specific embodiments, provided is a trispecific antibody comprising a PSMA antibody provided herein in a knob-in-hole format. In specific embodiments, provided is a quadraspecific antibody comprising a PSMA antibody provided herein in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, a PSMA antibody provided herein is comprised in a bispecific antibody. In some embodiments, a PSMA antibody provided herein is comprised in a trispecific antibody. In some embodiments, a PSMA antibody provided herein is comprised in a quadraspecific antibody. In some embodiments, a PSMA bispecific antibody provided herein is comprised in a multispecific antibody.

In certain embodiments, a multispecific antibody provided herein comprises a first binding domain comprising a PSMA antibody provided herein that binds to a first PSMA epitope, and a second binding domain that binds to a second epitope, wherein the first PSMA epitope and the second epitope are not the same. In certain embodiments, a bispecific antibody provided herein comprises a first binding domain comprising a PSMA antibody provided herein that binds to a first PSMA epitope, and a second binding domain that binds to a second epitope, wherein the first PSMA epitope and the second epitope are not the same. In certain embodiments, a trispecific antibody provided herein comprises a first binding domain comprising a PSMA antibody provided herein that binds to a first PSMA epitope, a second binding domain that binds to a second epitope, and a third binding domain that binds to a third epitope, wherein the first PSMA epitope, the second epitope, and the third epitope are not the same. In certain embodiments, a quadraspecific antibody provided herein comprises a first binding domain comprising a PSMA antibody provided herein that binds to a first PSMA epitope, a second binding domain that binds to a second epitope, a third binding domain that binds to a third epitope, and a fourth binding domain that binds to a fourth epitope, wherein the first PSMA epitope, the second epitope, the third epitope, and the fourth epitope are not the same. In certain embodiments, a multispecific antibody provided herein comprises a first binding domain comprising a PSMA antibody provided herein that binds to a first PSMA antigen, and a second binding domain that binds to a second antigen, wherein the first PSMA antigen and the second antigen are not the same. In certain embodiments, a bispecific antibody provided herein comprises a first binding domain comprising a PSMA antibody provided herein that binds to a first PSMA antigen, and a second binding domain that binds to a second antigen, wherein the first PSMA antigen and the second antigen are not the same. In certain embodiments, a trispecific antibody provided herein comprises a first binding domain comprising a PSMA antibody provided herein that binds to a first PSMA antigen, a second binding domain that binds to a second antigen, and a third binding domain that binds to a third antigen, wherein the first PSMA antigen, the second antigen, and the third antigen are not the same. In certain embodiments, a quadraspecific antibody provided herein comprises a first binding domain comprising a PSMA antibody provided herein that binds to a first PSMA antigen, a second binding domain that binds to a second antigen, a third binding domain that binds to a third antigen, and a fourth binding domain that binds to a fourth antigen, wherein the first PSMA antigen, the second antigen, the third antigen, and the fourth antigen are not the same. In a specific embodiment, a PSMA antibody, or antigen binding fragment thereof, provided herein specifically binds to PSMA.

In some embodiments, the multispecific antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the PSMA multispecific antibodies or antigen binding fragments thereof bind to a first epitope located on PSMA and a second epitope of a second target antigen. In some embodiments, provided herein is a multispecific antibody comprising: (a) a first binding domain that binds to a PSMA antigen, and (b) a second binding domain that binds to a second target antigen. In some embodiments, provided herein is a multispecific antibody comprising: (a) a first binding domain that specifically binds to a PSMA antigen, and (b) a second binding domain that specifically binds to a second target antigen. In some embodiments, provided herein is a multispecific antibody comprising: (a) a first binding domain that binds to a first epitope on a PSMA antigen, and (b) a second binding domain that binds to a second epitope on a second target antigen. In some embodiments, provided herein is a multispecific antibody comprising: (a) a first binding domain that specifically binds to a first epitope on a PSMA antigen, and (b) a second binding domain that specifically binds to a second epitope on a second target antigen.

In specific embodiments, the PSMA antigen is on the surface of a T cell. In certain embodiments, the second target antigen is not PSMA. The binding of the PSMA multispecific antibody to PSMA present on the surface of the T cell, and the binding of the second target antigen present on the surface of the second target cell can, for example, result in the killing of the second target cell. In other embodiment, the binding of the PSMA multispecific antibody to PSMA present on the surface of the T cell, and the binding of a second target antigen can, for example, result in the activation of the T cell.

In another aspect, provided herein is a multispecific antibody that comprises a first binding domain that binds to PSMA and a second binding domain that binds to CD3 (“multispecific PSMAxCD3 antibody”). In some embodiments, the multispecific PSMAxCD3 antibody is a bispecific antibody. In some embodiments, the multispecific PSMAxCD3 antibody is a trispecific antibody. In some embodiments, the multispecific PSMAxCD3 antibody is a quadraspecific antibody.

In one embodiment, the multispecific PSMAxCD3 antibody comprises: (a) a first binding domain that binds PSMA, and (b) a second binding domain that binds to CD3. In one embodiment, the multispecific PSMAxCD3 antibody comprises: (a) a first binding domain that binds PSMA, and (b) a second binding domain that binds to CD3, and (c) a third binding domain that binds to a third target. In one embodiment, the multispecific PSMAxCD3 antibody comprises: (a) a first binding domain that binds PSMA, and (b) a second binding domain that binds to CD3, (c) a third binding domain that binds to a third target, and (d) a fourth binding domain that binds to a fourth target.

In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment of the multispecific PSMAxCD3 antibodies provided herein, the first binding domain that binds PSMA comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814.

In some embodiments of the multispecific PSMAxCD3 antibodies provided herein, the VH CDR1, VH CDR2, VH CDR3, VL CDR1, VL CDR2, and VL CDR3 amino acid sequences of the first binding domain that binds PSMA are according to the Kabat numbering system. In some embodiments of the multispecific PSMAxCD3 antibodies provided herein, the VH CDR1, VH CDR2, VH CDR3, VL CDR1, VL CDR2, and VL CDR3 amino acid sequences of the first binding domain that binds PSMA are according to the Chothia numbering system. In some embodiments of the multispecific PSMAxCD3 antibodies provided herein, the VH CDR1, VH CDR2, VH CDR3, VL CDR1, VL CDR2, and VL CDR3 amino acid sequences of the first binding domain that binds PSMA are according to the AbM numbering system. In some embodiments of the multispecific PSMAxCD3 antibodies provided herein, the VH CDR1, VH CDR2, VH CDR3, VL CDR1, VL CDR2, and VL CDR3 amino acid sequences of the first binding domain that binds PSMA are according to the Contact numbering system. In some embodiments of the multispecific PSMAxCD3 antibodies provided herein, the VH CDR1, VH CDR2, VH CDR3, VL CDR1, VL CDR2, and VL CDR3 amino acid sequences of the first binding domain that binds PSMA are according to the IMGT numbering system. In some embodiments, the VH CDR1, VH CDR2, VH CDR3, VL CDR1, VL CDR2, and VL CDR3 amino acid sequences of the first binding domain that binds PSMA are according to a combination of the numbering systems provided herein.

In some embodiments of the multispecific PSMAxCD3 antibodies provided herein, the first binding domain binds a PSMA antigen. In some embodiments of the multispecific PSMAxCD3 antibodies provided herein, the first binding domain binds a PSMA epitope. In some embodiments of the multispecific PSMAxCD3 antibodies provided herein, the first binding domain specifically binds to PSMA. In some embodiments of the multispecific PSMAxCD3 antibodies provided herein, the VH CDR1, VH CDR2, VH CDR3, VL CDR1, VL CDR2 and VL CDR3 of the first binding domain form a binding site for an antigen of the PSMA. In some embodiments of the multispecific PSMAxCD3 antibodies provided herein, the VH CDR1, VH CDR2, VH CDR3, VL CDR1, VL CDR2 and VL CDR3 of the first binding domain form a binding site for an epitope of the PSMA. In some embodiments of the multispecific PSMAxCD3 antibodies provided herein, the PSMA is present on the surface of a T cell.

In some embodiments of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:847; and (ii) a VL comprising a VL CDR1, a VL CDR2, and a VL CDR3 having an amino acid sequence of a VL CDR1, a VL CDR2, and a VL CDR3, respectively, of SEQ ID NO:848. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:847; and (ii) a VL comprising a VL CDR1, a VL CDR2, and a VL CDR3 having an amino acid sequence of a VL CDR1, a VL CDR2, and a VL CDR3, respectively, of SEQ ID NO:848. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:915; and (ii) a VL comprising a VL CDR1, a VL CDR2, and a VL CDR3 having an amino acid sequence of a VL CDR1, a VL CDR2, and a VL CDR3, respectively, of SEQ ID NO:916. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:915; and (ii) a VL comprising a VL CDR1, a VL CDR2, and a VL CDR3 having an amino acid sequence of a VL CDR1, a VL CDR2, and a VL CDR3, respectively, of SEQ ID NO:916. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:983; and (ii) a VL comprising a VL CDR1, a VL CDR2, and a VL CDR3 having an amino acid sequence of a VL CDR1, a VL CDR2, and a VL CDR3, respectively, of SEQ ID NO:984. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:983; and (ii) a VL comprising a VL CDR1, a VL CDR2, and a VL CDR3 having an amino acid sequence of a VL CDR1, a VL CDR2, and a VL CDR3, respectively, of SEQ ID NO:984. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:1463; and (ii) a VL comprising a VL CDR1, a VL CDR2, and a VL CDR3 having an amino acid sequence of a VL CDR1, a VL CDR2, and a VL CDR3, respectively, of SEQ ID NO:1464. In one embodiment of the multispecific PSMAxCD3 antibodies provided herein, the second binding domain that binds CD3 comprises: (i) a VH comprising a VH CDR1, a VH CDR2, and a VH CDR3 having an amino acid sequence of a VH CDR1, a VH CDR2, and a VH CDR3, respectively, of SEQ ID NO:1505; and (ii) a VL comprising a VL CDR1, a VL CDR2, and a VL CDR3 having an amino acid sequence of a VL CDR1, a VL CDR2, and a VL CDR3, respectively, of SEQ ID NO:1464.

In some embodiments of the multispecific PSMAxCD3 antibodies provided herein, the VH CDR1, VH CDR2, VH CDR3, VL CDR1, VL CDR2, and VL CDR3 amino acid sequences of the second binding domain that binds CD3 are according to the Kabat numbering system. In some embodiments of the multispecific PSMAxCD3 antibodies provided herein, the VH CDR1, VH CDR2, VH CDR3, VL CDR1, VL CDR2, and VL CDR3 amino acid sequences of the second binding domain that binds CD3 are according to the Chothia numbering system. In some embodiments of the multispecific PSMAxCD3 antibodies provided herein, the VH CDR1, VH CDR2, VH CDR3, VL CDR1, VL CDR2, and VL CDR3 amino acid sequences of the second binding domain that binds CD3 are according to the AbM numbering system. In some embodiments of the multispecific PSMAxCD3 antibodies provided herein, the VH CDR1, VH CDR2, VH CDR3, VL CDR1, VL CDR2, and VL CDR3 amino acid sequences of the second binding domain that binds CD3 are according to the Contact numbering system. In some embodiments of the multispecific PSMAxCD3 antibodies provided herein, the VH CDR1, VH CDR2, VH CDR3, VL CDR1, VL CDR2, and VL CDR3 amino acid sequences of the second binding domain that binds CD3 are according to the IMGT numbering system. In some embodiments, the VH CDR1, VH CDR2, VH CDR3, VL CDR1, VL CDR2, and VL CDR3 amino acid sequences of the second binding domain that binds CD3 are according to a combination of the numbering systems provided herein.

In some embodiments of the multispecific PSMAxCD3 antibodies provided herein, the second binding domain binds a CD3 antigen. In some embodiments of the multispecific PSMAxCD3 antibodies provided herein, the second binding domain binds a CD3 epitope. In some embodiments of the multispecific PSMAxCD3 antibodies provided herein, the second binding domain specifically binds to CD3. In some embodiments of the multispecific PSMAxCD3 antibodies provided herein, the VH CDR1, VH CDR2, VH CDR3, VL CDR1, VL CDR2 and VL CDR3 of the second binding domain form a binding site for an antigen of the CD3. In some embodiments, the VH CDR1, VH CDR2, VH CDR3, VL CDR1, VL CDR2 and VL CDR3 of the second binding domain form a binding site for an epitope of the CD3. In some embodiments, the CD3 is present on the surface of a T cell.

In some embodiments of the multispecific PSMAxCD3 antibodies provided herein, the third target is not a PSMA antigen. In some embodiments of the multispecific PSMAxCD3 antibodies provided herein, the fourth target is not a PSMA antigen. In some embodiments of the multispecific PSMAxCD3 antibodies provided herein, the third target is not a PSMA antigen, and the fourth target is not a PSMA antigen. In some embodiments of the multispecific PSMAxCD3 antibodies provided herein, the third target is not a CD3 antigen. In some embodiments of the multispecific PSMAxCD3 antibodies provided herein, the fourth target is not a CD3 antigen. In some embodiments of the multispecific PSMAxCD3 antibodies provided herein, the third target is not a CD3 antigen, and the fourth target is not a CD3 antigen. In some embodiments of the multispecific PSMAxCD3 antibodies provided herein, the third target is not a PSMA epitope. In some embodiments of the multispecific PSMAxCD3 antibodies provided herein, the fourth target is not a PSMA epitope. In some embodiments of the multispecific PSMAxCD3 antibodies provided herein, the third target is not a PSMA epitope, and the fourth target is not a PSMA epitope. In some embodiments of the multispecific PSMAxCD3 antibodies provided herein, the third target is not a CD3 epitope. In some embodiments of the multispecific PSMAxCD3 antibodies provided herein, the fourth target is not a CD3 epitope. In some embodiments of the multispecific PSMAxCD3 antibodies provided herein, the third target is not a CD3 epitope, and the fourth target is not a CD3 epitope.

In a specific embodiment, the target is from a mammal. In a specific embodiment, the target is from a rat. In a specific embodiment, the target is from a mouse. In a specific embodiment, the target is from a primate. In a specific embodiment, the target is from a human.

In specific embodiments, provided is a multispecific PSMAxCD3 antibody in a knob-in-hole format. In specific embodiments, provided is a bispecific PSMAxCD3 antibody in a knob-in-hole format. In specific embodiments, provided is a trispecific antibody in a knob-in-hole format. In specific embodiments, provided is a quadraspecific antibody in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, a PSMAxCD3 antibody provided herein is comprised in a bispecific antibody. In some embodiments, a PSMAxCD3 antibody provided herein is comprised in a trispecific antibody. In some embodiments, a PSMAxCD3 antibody provided herein is comprised in a quadraspecific antibody. In some embodiments, a PSMAxCD3 bispecific antibody provided herein is comprised in a multispecific antibody.

In certain embodiments, a trispecific PSMAxCD3 antibody provided herein comprises a first binding domain comprising a PSMA antibody provided herein that binds to a PSMA epitope, a second binding domain comprising a CD3 antibody provided herein that that binds to a CD3 epitope, and a third binding domain that binds to a third epitope, wherein the PSMA epitope, the CD3 epitope, and the third epitope are not the same. In certain embodiments, a quadraspecific antibody provided herein comprises a first binding domain comprising a PSMA antibody provided herein that binds to a PSMA epitope, a second binding domain comprising a CD3 antibody provided herein that that binds to a CD3 epitope, a third binding domain that binds to a third epitope, and a fourth binding domain that binds to a fourth epitope, wherein the PSMA epitope, the CD3 epitope, the third epitope, and the fourth epitope are not the same. In certain embodiments, a trispecific antibody provided herein comprises a first binding domain comprising a PSMA antibody provided herein that binds to a PSMA antigen, a second binding domain comprising a CD3 antibody provided herein that that binds to a CD3 antigen, and a third binding domain that binds to a third antigen, wherein the PSMA antigen, the CD3 antigen, and the third antigen are not the same. In certain embodiments, a quadraspecific antibody provided herein that binds to a PSMA antigen, a second binding domain comprising a CD3 antibody provided herein that that binds to a CD3 antigen, a third binding domain that binds to a third antigen, and a fourth binding domain that binds to a fourth antigen, wherein the PSMA antigen, the CD3 antigen, the third antigen, and the fourth antigen are not the same. In certain embodiments of a multispecific PSMAxCD3 antibody provided herein, the first binding domain that binds to PSMA specifically binds to the PSMA. In other embodiments of a multispecific PSMAxCD3 antibody provided herein, the second binding domain that binds to CD3 specifically binds to the CD3. In yet other embodiments of a multispecific PSMAxCD3 antibody provided herein, the first binding domain that binds to PSMA specifically binds to the PSMA, and the second binding domain that binds to CD3 specifically binds to the CD3.

In some embodiments, the multispecific PSMAxCD3 antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the PSMA antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the PSMAxCD3 multispecific antibodies or antigen binding fragments thereof bind to a first epitope located on PSMA and a second epitope of located on CD3. In some embodiments, provided herein is a multispecific PSMAxCD3 antibody comprising: (a) a first binding domain that binds to a PSMA antigen, and (b) a second binding domain that binds to a CD3 antigen. In some embodiments, provided herein is a multispecific PSMAxCD3 antibody comprising: (a) a first binding domain that specifically binds to a PSMA antigen, and (b) a second binding domain that specifically binds to a CD3 antigen. In some embodiments, provided herein is a multispecific PSMAxCD3 antibody comprising: (a) a first binding domain that binds to a first epitope on a PSMA antigen, and (b) a second binding domain that binds to a second epitope on a CD3 antigen. In some embodiments, provided herein is a multispecific antibody comprising: (a) a first binding domain that specifically binds to a first epitope on a PSMA antigen, and (b) a second binding domain that specifically binds to a second epitope on a CD3 antigen.

In specific embodiments, the PSMA antigen is on the surface of a T cell. In specific embodiments, the CD3 antigen is on the surface of a T cell. The binding of the PSMAxCD3 multispecific antibody to PSMA and CD3 present on the surface of T cells can, for example, result in the killing of the cell. In other embodiments, the binding of the PSMAxCD3 multispecific antibody to PSMA and CD3 present on the surface of T cells can, for example, result in the activation of the T cell.

In some embodiments, the PSMA antibody comprises a single chain antibody. In some embodiments, the PSMA antibody comprises a single domain antibody. In certain embodiments, the PSMA antibody comprises a nanobody. In certain embodiments, the PSMA antibody comprises a VHH antibody. In certain embodiments, the PSMA antibody comprises a llama antibody.

In some embodiments, the PSMA multispecific antibody comprises a single chain antibody. In some embodiments, the PSMA multispecific antibody comprises a single domain antibody. In certain embodiments, the PSMA multispecific antibody comprises a nanobody. In certain embodiments, the PSMA multispecific antibody comprises a VHH antibody. In certain embodiments, the PSMA multispecific antibody comprises a llama antibody. In some embodiments, the PSMA multispecific antibody does not comprise a single chain antibody. In some embodiments, the PSMA multispecific antibody does not comprise a single domain antibody. In certain embodiments, the PSMA multispecific antibody does not comprise a nanobody. In certain embodiments, the PSMA multispecific antibody does not comprise a VHH antibody. In certain embodiments, the PSMA multispecific antibody does not comprise a llama antibody.

According to another particular aspect, provided herein is a PSMA antibody or antigen-binding fragment thereof that induces antibody-dependent cell-mediated cytotoxicity (ADCC). The antibody or antigen-binding fragment thereof can, for example, induce ADCC in vitro.

In certain embodiments, the antibody or antigen-binding fragment thereof induces T cell dependent cytotoxicity of a second cell in vitro with an EC₅₀ of less than about 160 pM, when assessed in vitro at an effector to target cell ratio of 1:1.

In some embodiments, CD3 is present on the surface of a T cell. In some embodiments, the CD3 is present on the surface of a T cell, and the second target antigen is PSMA on the surface of a second cell. In some embodiments, the second cell is killed when the multispecific antibody binds to the CD3 on the surface of the T cell and the PSMA target antigen on the surface of the second cell. In some embodiments, the second cell is a prostate cell. In some embodiments, the second cell is a prostate cancer cell. In some embodiments, the second cell is a renal cell. In some embodiments, the second cell is a renal cancer cell.

In some embodiments, the multispecific antibody induces T cell dependent cytotoxicity of the second cell in vitro with an EC₅₀ of less than about 500 pM. In some embodiments, the multispecific antibody induces T cell dependent cytotoxicity of the second cell in vitro with an EC₅₀ of less than about 300 pM. In some embodiments, the multispecific antibody induces γδ T cell dependent cytotoxicity of the second cell in vitro with an EC₅₀ of less than about 160 pM. In some embodiments, the EC₅₀ is assessed with a mixture of γδ T effector cells and target cells expressing the second target antigen. In some embodiments, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In certain embodiments, the EC₅₀ is less than about 1000 pM, less than about 900 pM, less than about 800 pM, less than about 700 pM, less than about 600 pM, less than about 500 pM, less than about 400 pM, less than about 300 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 90 pM, less than about 80 pM, less than about 70 pM, less than about 60 pM, less than about 50 pM, less than about 40 pM, less than about 30 pM, less than about 20 pM, or less than about 10 pM.

In certain embodiments, the effector to target cell ratio can, for example, be 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1, 7:1, 8:1, 9:1, or 10:1. In certain embodiments, the concentration of the multispecific antibody or antigen-binding fragment thereof is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL.

In another aspect, provided herein is an antibody that competes for binding to PSMA with any of the PSMA antibodies described herein. In another aspect, provided herein is an antibody that binds to the same epitope as any of the PSMA antibodies described herein. In another aspect, provided is a PSMA antibody that binds an epitope on PSMA that overlaps with the epitope on PSMA bound by a PSMA antibody described herein. In some embodiments, the PSMA antibody comprises a VH CDR1, VH CDR2, and VH CDR3 of a PSMA antibody provided herein. In some embodiments, the PSMA antibody comprises a VL CDR1, VL CDR2, and VL CDR3 of a PSMA antibody provided herein. In some embodiments, the PSMA antibody comprises a VH CDR1, VH CDR2, VH CDR3, a VL CDR1, VL CDR2, and VL CDR3 of a PSMA antibody provided herein. In some embodiments, the PSMA antibody comprises a VH of a PSMA antibody provided herein. In some embodiments, the PSMA antibody comprises a VL of a PSMA antibody provided herein. In some embodiments, the PSMA antibody comprises a VH and a VL of a PSMA antibody provided herein. In some embodiments, the PSMA antibody comprises a VH CDR1, VH CDR2, VH CDR3, a VL CDR1, VL CDR2, and VL CDR3 of a PSMA antibody provided herein. In some embodiments, the VH CDR1, VH CDR2, VH CDR3, VL CDR1, VL CDR2, and VL CDR3 amino acid sequences of the PSMA antibody are according to the Kabat numbering system. In some embodiments, the VH CDR1, VH CDR2, VH CDR3, VL CDR1, VL CDR2, and VL CDR3 amino acid sequences of the PSMA antibody are according to the Chothia numbering system. In some embodiments, the VH CDR1, VH CDR2, VH CDR3, VL CDR1, VL CDR2, and VL CDR3 amino acid sequences of the PSMA antibody are according to the AbM numbering system. In some embodiments, the VH CDR1, VH CDR2, VH CDR3, VL CDR1, VL CDR2, and VL CDR3 amino acid sequences of the PSMA antibody are according to the Contact numbering system. In some embodiments, the VH CDR1, VH CDR2, VH CDR3, VL CDR1, VL CDR2, and VL CDR3 amino acid sequences of the PSMA antibody are according to the IMGT numbering system. In some embodiments, the VH CDR1, VH CDR2, VH CDR3, VL CDR1, VL CDR2, and VL CDR3 amino acid sequences of the PSMA antibody are according to a combination of the numbering systems provided herein. In certain embodiments, the PSMA antibody is a multispecific antibody. In some embodiments, the PSMA antibody is a bispecific antibody.

In another aspect, provided is an antibody that competes for binding to PSMA with a PSMA reference antibody. In another aspect, provided is a PSMA antibody that binds to the same PSMA epitope as a PSMA reference antibody. In another aspect, provided is a PSMA antibody that binds an epitope on PSMA that overlaps with the epitope on PSMA bound by a PSMA reference antibody. In some embodiments, the PSMA reference antibody comprises a VH CDR1, VH CDR2, and VH CDR3 of a PSMA reference antibody provided herein. In some embodiments, the PSMA reference antibody comprises a VL CDR1, VL CDR2, and VL CDR3 of a PSMA reference antibody provided herein. In some embodiments, the PSMA reference antibody comprises a VH CDR1, VH CDR2, VH CDR3, a VL CDR1, VL CDR2, and VL CDR3 of a PSMA reference antibody provided herein. In some embodiments, the PSMA reference antibody comprises a VH of a PSMA reference antibody provided herein. In some embodiments, the PSMA reference antibody comprises a VL of a PSMA reference antibody provided herein. In some embodiments, the PSMA reference antibody comprises a VH and a VL of a PSMA reference antibody provided herein. In some embodiments, the PSMA reference antibody comprises a VH CDR1, VH CDR2, VH CDR3, a VL CDR1, VL CDR2, and VL CDR3 of a PSMA reference antibody provided herein. In some embodiments, the VH CDR1, VH CDR2, VH CDR3, VL CDR1, VL CDR2, and VL CDR3 amino acid sequences of the PSMA reference antibody are according to the Kabat numbering system. In some embodiments, the VH CDR1, VH CDR2, VH CDR3, VL CDR1, VL CDR2, and VL CDR3 amino acid sequences of the PSMA reference antibody are according to the Chothia numbering system. In some embodiments, the VH CDR1, VH CDR2, VH CDR3, VL CDR1, VL CDR2, and VL CDR3 amino acid sequences of the PSMA reference antibody are according to the AbM numbering system. In some embodiments, the VH CDR1, VH CDR2, VH CDR3, VL CDR1, VL CDR2, and VL CDR3 amino acid sequences of the PSMA reference antibody are according to the Contact numbering system. In some embodiments, the VH CDR1, VH CDR2, VH CDR3, VL CDR1, VL CDR2, and VL CDR3 amino acid sequences of the PSMA reference antibody are according to the IMGT numbering system. In some embodiments, the VH CDR1, VH CDR2, VH CDR3, VL CDR1, VL CDR2, and VL CDR3 amino acid sequences of the PSMA reference antibody are according to a combination of the numbering systems provided herein. In certain embodiments, the antibody is a multispecific antibody. In some embodiments, the antibody is a bispecific antibody. In certain embodiments, the PSMA reference antibody is a multispecific antibody. In some embodiments, the PSMA reference antibody is a bispecific antibody.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds PSMA, the second antigen binding domain specifically binds a second target, and the third antigen binding domain specifically binds a third target. In certain embodiments, the second target is CD3.

In some embodiments, the multispecific PSMAxCD3 antibody activates CD3+ T cells. In some embodiments, the multispecific PSMAxCD3 antibody binds to PSMA on prostate cells and binds to CD3 on T cells. In certain embodiments, the multispecific PSMAxCD3 antibody recruits CD3+ T cells to PSMA-expressing cells. In certain embodiments, the multispecific PSMAxCD3 antibody induces T cell proliferation. In certain embodiments, the multispecific PSMAxCD3 antibody induces T cell-meditated killing of the PSMA-expressing cells. In some embodiments, the multispecific PSMAxCD3 antibody specifically binds PSMA and the CD3 with an affinity that results in activation or recruitment of CD3+ T cells only upon co-engagement of the CD3 and PSMA. In specific embodiments, the PSMA-expressing cells are prostate cells. In some embodiments, the PSMA-expressing cells are prostate cancer cells. In some embodiments, the PSMA-expressing cells are renal cells. In some embodiments, the PSMA-expressing cells are renal cancer cells.

In some embodiments described herein, immune effector properties of the antibodies provided herein can be enhanced or silenced through Fc modifications by techniques known to those skilled in the art. For example, Fc effector functions such as Clq binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. can be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such Abs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng 88:901-908, 2004), or coexpression of β-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the antibodies provided herein can also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcgRs) on the surface of immune effector cells such as natural killers and macrophages, leading to the phagocytosis or lysis of the targeted cells. In CDC, the antibodies kill the targeted cells by triggering the complement cascade at the cell surface. The antibodies described herein include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcgRs or the complement factors. The binding of IgG to the activating (FcgRI, FcgRIIa, FcgRIIIa and FcgRIIIb) and inhibitory (FcgRIIb) FcgRs or the first component of complement (Clq) depends on residues located in the hinge region and the CH2 domain. Mutations have been introduced in IgG1, IgG2 and IgG4 to reduce or silence Fc functionalities. The antibodies described herein can include these modifications.

In one embodiment, the antibody comprises an Fc region with one or more of the following properties: (a) reduced effector function when compared to the parent Fc; (b) reduced affinity to Fcg RI, Fcg RIIa, Fcg RIIb, Fcg RIIIb and/or Fcg RIIIa, (c) reduced affinity to FcgRI (d) reduced affinity to FcgRIIa (e) reduced affinity to FcgRIIb, (f) reduced affinity to Fcg RIIIb or (g) reduced affinity to FcgRIIIa.

In some embodiments, the antibodies or antigen-binding fragments are IgG, or derivatives thereof, e.g., IgG1, IgG2, IgG3, and IgG4 isotypes. In some embodiments wherein the antibody has an IgG4 isotype, the antibody contains S228P, L234A, and L235A substitutions in its Fc region. In some embodiments wherein the antibody has an IgG1 isotype, the antibody contains S228P, L234A, and L235A substitutions in its Fc region. The antibodies described herein can include these modifications. In some embodiments, the antibody has an IgG1 isotype.

In some embodiments, the antibody is of IgG4 isotype, optionally comprising a heavy chain substitution S228P when compared to the wild type IgG4.

In some embodiments, the antibody is of IgG1 isotype, optionally comprising heavy chain substitutions L234A, G237A, P238S, H268A, V309L, A330S and P331S when compared to the wild type IgG1.

In some embodiments, a PSMA antibody provided herein is chimeric. In some embodiments, a PSMA antibody provided herein is human. In some embodiments, a PSMA antibody provided herein is humanized. In certain embodiments, a PSMA antibody provided herein is an isolated PSMA antibody. In some embodiments, a PSMA antigen binding fragment provided herein is chimeric. In some embodiments, a PSMA antigen binding fragment provided herein is human. In some embodiments, a PSMA antigen binding fragment provided herein is humanized. In certain embodiments, a PSMA antigen binding fragment provided herein is an isolated PSMA antigen binding fragment. In some embodiments, a PSMA antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, a PSMA antibody provided herein is multivalent. In some embodiments, the PSMA antibody is capable of binding at least three antigens. In some embodiments, the PSMA antibody is capable of binding at least four antigens. In some embodiments, the PSMA antibody is capable of binding at least five antigens.

In some embodiments, a PSMA multispecific antibody provided herein is chimeric. In some embodiments, a PSMA multispecific antibody provided herein is human. In some embodiments, a PSMA multispecific antibody provided herein is humanized. In certain embodiments, a PSMA multispecific antibody provided herein is an isolated PSMA multispecific antibody. In some embodiments, a PSMA multispecific antibody comprising a PSMA antigen binding fragment provided herein is chimeric. In some embodiments, a PSMA multispecific antibody comprising a PSMA antigen binding fragment provided herein is human. In some embodiments, a PSMA multispecific antibody comprising a PSMA antigen binding fragment provided herein is humanized. In certain embodiments, a PSMA multispecific antibody comprising a PSMA antigen binding fragment provided herein is an isolated PSMA multispecific antibody. In certain embodiments, the PSMA multispecific antibody is a multispecific PSMAxCD3 antibody.

In some embodiments of the PSMA multispecific antibodies provided herein, the first binding domain is human. In some embodiments, the second binding domain is human. In some embodiments of the PSMA multispecific antibodies provided herein, both the first binding domain and the second binding domain are human. In some embodiments of the PSMA multispecific antibodies provided herein, the first binding domain is humanized. In some embodiments of the PSMA multispecific antibodies provided herein, the second binding domain is humanized. In some embodiments of the PSMA multispecific antibodies provided herein, both the first binding domain and the second binding domain are humanized. In some embodiments of the PSMA multispecific antibodies provided herein, both the first binding domain is human and the second binding domain is humanized. In some embodiments of the PSMA multispecific antibodies provided herein, both the first binding domain is humanized and the second binding domain is human. In certain embodiments, the PSMA multispecific antibody is a multispecific PSMAxCD3 antibody.

In some embodiments, a PSMA multispecific antibody provided herein is multivalent. In some embodiments, the multispecific antibody is capable of binding at least three antigens. In some embodiments, the multispecific antibody is capable of binding at least five antigens. In certain embodiments, the multispecific antibody is a multispecific antibody. In some embodiments, a PSMA multispecific antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In certain embodiments, the PSMA multispecific antibody is a multispecific PSMAxCD3 antibody.

In certain embodiments, the antibodies provided herein are part of a multispecific antibody. In some embodiments, the multispecific antibody comprises a first binding domain that binds to a PSMA antigen. In some embodiments, the multispecific antibody comprises a first binding domain that binds to a PSMA antigen and comprises a second binding domain that binds to a second target antigen, as provided herein. In certain embodiments, the multispecific antibody binds to a PSMA antigen, a second target antigen, and one or more additional antigens. In some embodiments of the various antibodies provided herein, the antibody binds to an epitope of a given antigen. In certain embodiments, the multispecific PSMA antibody is a multispecific PSMAxCD3 antibody, wherein the second target is CD3.

Variants of the antibodies specifically binding PSMA described herein are also contemplated as embodiments. In some embodiments, antibodies provided herein have altered amino acid sequences when compared to the parental antibodies can be generated using standard cloning and expression technologies. For example, site-directed mutagenesis or PCR-mediated mutagenesis can be performed to introduce the mutation(s) and the effect on antibody binding or other property of interest can be evaluated using well known methods and the methods described herein and in the Examples. For example, variants can comprise one, two, three, four, five, six, seven, eight, nine, ten, eleven, twelve, thirteen, fourteen or fifteen amino acid substitutions in the VH and/or the VL as long as the homologous antibodies retain or have improved functional properties when compared to the parental antibodies. In one embodiment, a variant of a PSMA antibody provided herein comprises a VH with one amino acid substitution. In one embodiment, a variant of a PSMA antibody provided herein comprises a VH with two amino acid substitutions. In another embodiment, a variant of a PSMA antibody provided herein comprises a VH with three amino acid substitutions. In another embodiment, a variant of a PSMA antibody provided herein comprises a VH with four amino acid substitutions. In one embodiment, a variant of a PSMA antibody provided herein comprises a VH with five amino acid substitutions. In another embodiment, a variant of a PSMA antibody provided herein comprises a VH with six amino acid substitutions. In another embodiment, a variant of a PSMA antibody provided herein comprises a VH with seven amino acid substitutions. In one embodiment, a variant of a PSMA antibody provided herein comprises a VH with eight amino acid substitutions. In another embodiment, a variant of a PSMA antibody provided herein comprises a VH with nine amino acid substitutions. In another embodiment, a variant of a PSMA antibody provided herein comprises a VH with ten amino acid substitutions. In one embodiment, a variant of a PSMA antibody provided herein comprises a VH with eleven amino acid substitutions. In another embodiment, a variant of a PSMA antibody provided herein comprises a VH with twelve amino acid substitutions. In another embodiment, a variant of a PSMA antibody provided herein comprises a VH with thirteen amino acid substitutions. In one embodiment, a variant of a PSMA antibody provided herein comprises a VH with fourteen amino acid substitutions. In one embodiment, a variant of a PSMA antibody provided herein comprises a VH with fifteen amino acid substitutions. In another embodiment, a variant of a PSMA antibody provided herein comprises a VL with one amino acid substitution. In one embodiment, a variant of a PSMA antibody provided herein comprises a VL with two amino acid substitutions. In another embodiment, a variant of a PSMA antibody provided herein comprises a VL with three amino acid substitutions. In another embodiment, a variant of a PSMA antibody provided herein comprises a VL with four amino acid substitutions. In one embodiment, a variant of a PSMA antibody provided herein comprises a VL with five amino acid substitutions. In another embodiment, a variant of a PSMA antibody provided herein comprises a VL with six amino acid substitutions. In another embodiment, a variant of a PSMA antibody provided herein comprises a VL with seven amino acid substitutions. In one embodiment, a variant of a PSMA antibody provided herein comprises a VL with eight amino acid substitutions. In another embodiment, a variant of a PSMA antibody provided herein comprises a VL with nine amino acid substitutions. In another embodiment, a variant of a PSMA antibody provided herein comprises a VL with ten amino acid substitutions. In one embodiment, a variant of a PSMA antibody provided herein comprises a VL with eleven amino acid substitutions. In another embodiment, a variant of a PSMA antibody provided herein comprises a VL with twelve amino acid substitutions. In another embodiment, a variant of a PSMA antibody provided herein comprises a VL with thirteen amino acid substitutions. In one embodiment, a variant of a PSMA antibody provided herein comprises a VL with fourteen amino acid substitutions. In one embodiment, a variant of a PSMA antibody provided herein comprises a VL with fifteen amino acid substitutions. In specific embodiments, the variation of the variant compared to the parental antibody is not within the CDRs of the variant. In certain embodiments, the amino acid substitution is a conservative modification.

In some embodiments, the sequence identity can be about 90%, 91%, 92%, 93%, 94%, 95%, 96%, 97%, 98% or 99% to a VH or the VL amino acid sequences provided herein. In some embodiments, a variant of a PSMA antibody provided herein comprises a VH having about 90% sequence identity. In some embodiments, a variant of a PSMA antibody provided herein comprises a VH having about 91% sequence identity. In some embodiments, a variant of a PSMA antibody provided herein comprises a VH having about 92% sequence identity. In some embodiments, a variant of a PSMA antibody provided herein comprises a VH having about 93% sequence identity. In some embodiments, a variant of a PSMA antibody provided herein comprises a VH having about 94% sequence identity. In some embodiments, a variant of a PSMA antibody provided herein comprises a VH having about 95% sequence identity. In some embodiments, a variant of a PSMA antibody provided herein comprises a VH having about 96% sequence identity. In some embodiments, a variant of a PSMA antibody provided herein comprises a VH having about 97% sequence identity. In some embodiments, a variant of a PSMA antibody provided herein comprises a VH having about 98% sequence identity. In some embodiments, a variant of a PSMA antibody provided herein comprises a VH having about 99% sequence identity. In some embodiments, a variant of a PSMA antibody provided herein comprises a VL having about 90% sequence identity. In some embodiments, a variant of a PSMA antibody provided herein comprises a VL having about 91% sequence identity. In some embodiments, a variant of a PSMA antibody provided herein comprises a VL having about 92% sequence identity. In some embodiments, a variant of a PSMA antibody provided herein comprises a VL having about 93% sequence identity. In some embodiments, a variant of a PSMA antibody provided herein comprises a VL having about 94% sequence identity. In some embodiments, a variant of a PSMA antibody provided herein comprises a VL having about 95% sequence identity. In some embodiments, a variant of a PSMA antibody provided herein comprises a VL having about 96% sequence identity. In some embodiments, a variant of a PSMA antibody provided herein comprises a VL having about 97% sequence identity. In some embodiments, a variant of a PSMA antibody provided herein comprises a VL having about 98% sequence identity. In some embodiments, a variant of a PSMA antibody provided herein comprises a VL having about 99% sequence identity. In specific embodiments, the variation of the variant compared to the parental antibody is not within the CDRs of the variant.

In some embodiments, a multispecific antibody provided herein is a diabody, a cross-body, or a multispecific antibody obtained via a controlled Fab arm exchange as those described herein.

### Generation of Monospecific Antibodies

In some embodiments the PSMA antibodies are human. In some embodiments the PSMA antibodies are humanized.

Monospecific antibodies described herein (e.g., antibodies specifically binding PSMA) can be generated using various technologies. For example, the hybridoma method of Kohler and Milstein, Nature 256:495, 1975 can be used to generate monoclonal antibodies. In the hybridoma method, a mouse or other host animal, such as a hamster, rat or monkey, is immunized with human chimpanzee or macaque PSMA or CD3 or fragments of PSMA or CD3, such as the extracellular domain of PSMA or CD3, followed by fusion of spleen cells from immunized animals with myeloma cells using standard methods to form hybridoma cells (Goding, Monoclonal Antibodies: Principles and Practice, pp. 59-103 (Academic Press, 1986)). Colonies arising from single immortalized hybridoma cells are screened for production of antibodies with desired properties, such as specificity of binding, cross-reactivity or lack thereof, and affinity for the antigen.

Various host animals can be used to produce the PSMA antibodies described herein. For example, Balb/c mice can be used to generate mouse anti-human PSMA antibodies. The antibodies made in Balb/c mice and other non-human animals can be humanized using various technologies to generate more human-like sequences.

Exemplary humanization techniques including selection of human acceptor frameworks are known and include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs of parental antibodies are transferred onto human frameworks that can be selected based on their overall homology to the parental frameworks, based on similarity in CDR length, or canonical structure identity, or a combination thereof.

Humanized antibodies can be further optimized to improve their selectivity or affinity to a desired antigen by incorporating altered framework support residues to preserve binding affinity (back mutations) by techniques such as those described in Int. Patent Publ. Nos. WO1090/007861 and WO1992/22653, or by introducing variation at any of the CDRs for example to improve affinity of the antibody.

The framework sequences of the parental and engineered antibodies can further be modified, for example by back mutations to restore and/or improve binding of the generated antibodies to the antigen as described for example in U.S. Pat. No. 6,180,370. The framework sequences of the parental or engineered antibodies can further be modified by mutating one or more residues within the framework region (or alternatively within one or more CDR regions) to remove T-cell epitopes to thereby reduce the potential immunogenicity of the antibody. This approach is also referred to as “deimmunization” and described in further detail in U.S. Patent Publ. No. US20070014796.

The CDR residues of the antibodies provided herein can be mutated to modulate affinity of the antibodies to PSMA and/or CD3.

The CDR residues of the antibodies provided herein can be mutated to minimize risk of post-translational modifications. Amino acid residues of putative motifs for deamination (NS), acid-catalyzed hydrolysis (DP), isomerization (DS), or oxidation (W) can be substituted with any of the naturally occurring amino acids to mutagenize the motifs, and the resulting antibodies can be tested for their functionality and stability using methods described herein.

Antibodies provided herein modified to improve stability, selectivity, cross-reactivity, affinity, immunogenicity or other desirable biological or biophysical property are contemplated. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn et al., (2001) J Mol Biol 305:989-1010). Potential structure destabilizing residues can be identified based upon the crystal structure of the antibody or by molecular modeling in certain cases, and the effect of the residues on antibody stability can be tested by generating and evaluating variants harboring mutations in the identified residues. One of the ways to increase antibody stability is to raise the thermal transition midpoint (T m) as measured by differential scanning calorimetry (DSC). In general, the protein Tm is correlated with its stability and inversely correlated with its susceptibility to unfolding and denaturation in solution and the degradation processes that depend on the tendency of the protein to unfold (Remmele et al., (2000) Biopharm 13:36-46). A number of studies have found correlation between the ranking of the physical stability of formulations measured as thermal stability by DSC and physical stability measured by other methods (Gupta et al., (2003) AAPS PharmSci 5E8; Zhang et al., (2004) J Pharm Sci 93:3076-89; Maa et al., (1996) Int J Pharm 140:155-68; Bedu-Addo et al., (2004) Pharm Res 21:1353-61; Remmele et al., (1997) Pharm Res 15:200-8). Formulation studies suggest that a Fab Tm has implication for long-term physical stability of a corresponding mAb.

C-terminal lysine (CTL) can be removed from injected antibodies by endogenous circulating carboxypeptidases in the blood stream (Cai et al., (2011) Biotechnol Bioeng 108:404-412). During manufacturing, CTL removal can be controlled to less than the maximum level by control of concentration of extracellular Zn²⁺, EDTA or EDTA-Fe³⁺ as described in U.S. Patent Publ. No. US20140273092. CTL content in antibodies can be measured using known methods.

Fc substitutions can be made to the antibodies provided herein to modulate antibody effector functions and/or pharmacokinetic properties. In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All these interactions are initiated through the binding of the Fc domain of antibodies or immune complexes to specialized cell surface receptors on cells. The diversity of cellular responses triggered by antibodies and immune complexes results from the heterogeneity of the Fc receptors: FcγRI (CD64), FcγRIIa (CD32A), and FcγRIII (CD16) are activating Fcγ receptors (i e., immune system enhancing) whereas FcγRIIb (CD32B) is an inhibitory Fcγ receptor (i.e., immune system dampening). Binding to the FcRn receptor modulates antibody half-life.

In some embodiments, the anti-PSMA antibodies or the bispecific anti-PSMA/anti-CD3 antibodies provided herein comprise at least one substitution in an Fc region.

In some embodiments, the anti-PSMA antibodies or the bispecific anti-PSMA/anti-CD3 antibodies provided herein comprise one, two, three, four, five, six, seven, eight, nine, ten, eleven, twelve, thirteen, fourteen or fifteen substitutions in the Fc region. As described elsewhere herein, Fc positions that can be substituted to modulate antibody half-life. Exemplary singular or combination substitutions that can be made to increase the half-life of the antibody are substitutions M428L/N434S, M252Y/S254T/T256E, T250Q/M428L, N434A and T307A/E380A/N434A. Exemplary singular or combination substitutions that can be made to reduce the half-life of the antibody are substitutions H435A, P2571/N434H, D376V/N434H, M252Y/S254T/T256E/H433K/N434F, T308P/N434A and H435R.

In some embodiments, the anti-PSMA antibodies or the bispecific anti-PSMA/anti-CD3 antibodies provided herein comprise at least one substitution in the Fc region selected from the group consisting of M428L/N434S, M252Y/S254T/T256E, T250Q/M428L, N434A, T307A/E380A/N434A, H435A, P257I/N434H, D376V/N434H, M252Y/S254T/T256E/H433K/N434F, T308P/N434A and H435R.

In some embodiments, the anti-PSMA antibodies or the bispecific anti-PSMA/anti-CD3 antibodies provided herein comprise at least one substitution in the Fc region that reduces binding of the antibody to an activating Fc-gamma receptor (FcγR) and/or reduces Fc effector functions such as Clq binding, complement dependent cytotoxicity (CDC), antibody-dependent cell-mediated cytotoxicity (ADCC) or phagocytosis (ADCP).

Fc positions that can be substituted to reduce binding of the antibody to the activating FcGR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4.

An S228P substitution can also be made in IgG4 antibodies to enhance IgG4 stability. In some embodiments, the anti-PSMA antibodies or the bispecific anti-PSMA/anti-CD3 antibodies provided herein comprises a S228P substitution, wherein residue numbering is according to the EU Index. In some embodiments, the anti-PSMA antibodies or the bispecific anti-PSMA/anti-CD3 antibodies provided herein comprise a F234A, a L235A or a F234A/L235A substitution, wherein residue numbering is according to the EU Index. In some embodiments, the anti-PSMA antibodies or the bispecific anti-PSMA/anti-CD3 antibodies provided herein comprise a S228P, a F234A and a L235A substitution, wherein residue numbering is according to the EU Index.

In other embodiments, transgenic animals, such as mice or rat carrying human immunoglobulin (Ig) loci in their genome can be used to generate human antibodies against a target protein, and are described in for example U.S. Pat. No. 6,150,584, Int. Patent Publ. No. WO99/45962, Int. Patent Publ. Nos. WO2002/066630, WO2002/43478, WO2002/043478 and WO1990/04036, Lonberg et al (1994) Nature 368:856-9; Green et al (1994) Nature Genet. 7:13-21; Green & Jakobovits (1998) Exp. Med. 188:483-95; Lonberg and Huszar (1995) Int Rev Immunol 13:65-93; Bruggemann et al., (1991) Eur J Immunol 21:1323-1326; Fishwild et al., (1996) Nat Biotechnol 14:845-851; Mendez et al., (1997) Nat Genet 15:146-156; Green (1999) J Immunol Methods 231:11-23; Yang et al., (1999) Cancer Res 59:1236-1243; Brtiggemann and Taussig (1997) Curr Opin Biotechnol 8:455-458. The endogenous immunoglobulin loci in such animal can be disrupted or deleted, and at least one complete or partial human immunoglobulin locus can be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc_net), KyMab (http://_www_kymab_com), Trianni (http://_www.trianni_com) and Ablexis (http://_www_ablexis_com) can be engaged to provide human antibodies directed against a selected antigen using technologies as described above.

Human antibodies can be selected from a phage display library, where the phage is engineered to express human immunoglobulins or portions thereof such as Fabs, single chain antibodies (scFv), or unpaired or paired antibody variable regions (Knappik et al., (2000) J Mol Biol 296:57-86; Krebs et al., (2001) J Immunol Meth 254:67-84; Vaughan et al., (1996) Nature Biotechnology 14:309-314; Sheets et al., (1998) PITAS (USA) 95:6157-6162; Hoogenboom and Winter (1991) J Mol Biol 227:381; Marks et al., (1991) J Mol Biol 222:581). The antibodies provided herein can be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries can be screened for phage binding to human and/or cyno PSMA or CD3 and the obtained positive clones can be further characterized, the Fabs isolated from the clone lysates, and expressed as full length IgGs. Such phage display methods for isolating human antibodies are described in for example: U.S. Pat. Nos. 5,223,409, 5,403,484, 5,571,698, 5,427,908, 5,580,717, 5,969,108, 6,172,197, 5,885,793; 6,521,404; 6,544,731; 6,555,313; 6,582,915 and 6,593,081.

Preparation of immunogenic antigens and monoclonal antibody production can be performed using any suitable technique, such as recombinant protein production. The immunogenic antigens can be administered to an animal in the form of purified protein, or protein mixtures including whole cells or cell or tissue extracts, or the antigen can be formed de novo in the animal's body from nucleic acids encoding said antigen or a portion thereof.

Generation of Multispecific PSMAxCD3 Antibodies

Multispecific PSMA antibodies, such as PSMAxCD3 bispecific antibodies provided herein can be generated by combining PSMA binding VH/VL domains with CD3 binding VH/VL domains isolated and characterized herein. Alternatively, the bispecific PSMAxCD3 antibodies can be engineered using VH/VL domains from publicly available monospecific anti-PSMA and anti-CD3 antibodies, and/or by mix-matching the PSMA or CD3 binding VH/VL domains identified herein with publicly available PSMA or CD3 binding VH/VL domains.

Exemplary anti-PSMA antibodies that can be used to engineer bispecific PSMAxCD3 molecules are for example those herein and in Tables 4-12 or 23-28. For example, the VH/VL domains of the PSMA antibodies provided herein can be incorporated into bispecific antibodies comprising CD3 binding VH/VL domains described herein and in Tables 16-22 or 23-28. For example, the VH/VL domains of the CD3 antibodies CD3B376, CD3B450, CD3B2030 and CD3W245 described herein (see, e.g., Tables 16-22) can be used to generate bispecific PSMAxCD3 antibodies.

Similarly, exemplary anti-CD3 antibodies that can be used to engineer bispecific PSMAxCD3 molecules are for example those described in Int. Patent Publ. Nos. WO2005/048935, WO2004/106380 and WO2015095392. These CD3 VH/VL domains can be incorporated into bispecific antibodies comprising PSMA binding VH/VL domains described herein and in Table 4-12 and 23-28. For example, the VH/VL domains of a PSMA antibody provided herein can be used to generate a PSMAxCD3 bispecific antibody. In certain embodiments, the PSMA antibody is PSMB889. In other embodiments, the PSMA antibody is PSMB890. In certain embodiments, the PSMA antibody is PSMB891. In certain embodiments, the PSMA antibody is PSMB892. In other embodiments, the PSMA antibody is PSMB893. In certain embodiments, the PSMA antibody is PSMB894. In other embodiments, the PSMA antibody is PSMB895. In certain embodiments, the PSMA antibody is PSMB896. In certain embodiments, the PSMA antibody is PSMB897. In other embodiments, the PSMA antibody is PSMB898. In certain embodiments, the PSMA antibody is PSMB899. In certain embodiments, the PSMA antibody is PSMHB49SC1133_011A11_1. In other embodiments, the PSMA antibody is PSMB896-G100A. In certain embodiments, the PSMA antibody is PSMA_P72_A10-HC-G54E. In certain embodiments, the PSMA antibody is PSMA_P72_D01-HC-D95E. In other embodiments, the PSMA antibody is PSMA_P72_F01. In other embodiments, the PSMA antibody is PSMA_P75_F01. In certain embodiments, the PSMA antibody is PSMA_P72_F07. In certain embodiments, the PSMA antibody is PSMA_P72_E07. In other embodiments, the PSMA antibody is PSMA_P72_D01. In certain embodiments, the PSMA antibody is PSMA_P72_C01. In other embodiments, the PSMA antibody is PSMA_P72_A10. In certain embodiments, the PSMA antibody is PSMA_P72_F02. In certain embodiments, the PSMA antibody is PSMA_P70_F02. In other embodiments, the PSMA antibody is PSMA_P72_G02. In other embodiments, the PSMA antibody is PSMA_P72_A11. In some embodiments, the PSMA antibody is PSMB946 (PSMB895 with a C-terminal Lys (K) amino acid residue). In some embodiments, the PSMA antibody is PSMB947 (PSMB896 with a C-terminal Lys (K) amino acid residue). In some embodiments, the PSMA antibody is PSMB948 (PSMB897 with a C-terminal Lys (K) amino acid residue). In some embodiments, the PSMA antibody is PSMB949 (PSMB898 with a C-terminal Lys (K) amino acid residue).

The generated bispecific PSMAxCD3 antibodies can be tested for their binding to PSMA and CD3, and for their desired functional characteristics, such as T cell mediated killing of PSMA-expressing cells (e.g., LNCaP).

Bispecific antibodies provided herein can comprise antibodies having a full length antibody structure. “Full length antibody” refers to an antibody having two full length antibody heavy chains and two full length antibody light chains. A full length antibody heavy chain (HC) consists of well-known heavy chain variable and constant domains VH, CH1, hinge, CH2, and CH3. A full length antibody light chain (LC) consists of well-known light chain variable and constant domains VL and CL. The full length antibody can be lacking the C-terminal lysine (K) in either one or both heavy chains. “Fab-arm” or “half molecule” refers to one heavy chain-light chain pair that specifically binds an antigen.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e. an epitope on PSMA and an epitope on CD3. Other methods of making multispecific antibodies are known and contemplated.

“Homodimerization” refers to an interaction of two heavy chains having identical CH3 amino acid sequences. “Homodimer” refers to an antibody having two heavy chains with identical CH3 amino acid sequences. “Heterodimerization” refers to an interaction of two heavy chains having non-identical CH3 amino acid sequences. “Heterodimer” refers to an antibody having two heavy chains with non-identical CH3 amino acid sequences.

As mentioned elsewhere, in some embodiments, the bispecific antibodies include designs such as the Triomab/Quadroma (Trion Pharma/Fresenius Biotech), Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody) (EMD Serono), the Biclonic (Merus) and the DuoBody (Genmab A/S).

In some embodiments, a PSMA multispecific antibody provided herein is in the knob-and-hole format. In some embodiments, a PSMA multispecific antibody provided herein is in a DuoBody format.

The Triomab quadroma technology can be used to generate full length bispecific antibodies provided herein. Triomab technology promotes Fab arm exchange between two parental chimeric antibodies, one parental mAb having IgG2a and the second parental mAb having rat IgG2b constant regions, yielding chimeric bispecific antibodies.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology can be used to generate full length bispecific antibodies provided herein. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies can be used to generate full length bispecific antibodies provided herein by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V_K409F_Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849.

LUZ-Y technology can be utilized to generate bispecific antibodies provided herein. In this technology, a leucine zipper is added into the C terminus of the CH3 domains to drive the heterodimer assembly from parental mAbs that is removed post-purification as described in Wranik et al., (2012) J Biol Chem 287(52): 42221-9.

SEEDbody technology can be utilized to generate bispecific antibodies provided herein. SEEDbodies have, in their constant domains, select IgG residues substituted with IgA residues to promote heterodimerszation as described in U.S. Patent No. US20070287170.

In some embodiments, also provided is a multispecific, multifunctional antibody that specifically binds to PSMA. Such a multispecific, multifunctional antibody that specifically binds to PSMA can be a trispecific antibody for dual targeting of tumor cells (e.g., trifunctional structures that can be designed to target two different targets/epitopes on the tumor cell and with the third functionality bind with high affinity to either T cells or NK-cells). Trispecific antibodies targeting two distinct tumor epitopes and engaging T- or NK-cells lyse the tumor cells that express both targets. Such molecules can be generated by antibody formats known in the art and are fully described. (WO2015/1842071, WO2015/158636, WO2010/136172, WO2013/174873). In a trispecific embodiment provided herein the antigen-binding polypeptide is bispecific for PSMA and a second distinct antigen on a tumor cell and additionally specific for an effector cell, in particular a T cell or a NK cell.

In addition to methods described above, bispecific antibodies provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, such as a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In some embodiments described herein, the bispecific antibody comprising a first domain specifically binding PSMA and a second domain specifically binding CD3 comprises at least one substitution in an antibody CH3 constant domain. In some embodiments, the at least one substitution in the antibody CH3 constant domain is 409R, F405L or F405L and R409K substitution, wherein residue numbering is according to the EU Index. Antibody domains and numbering are well known. “Asymmetrical” refers to non-identical substitutions in the two CH3 domains in two separate heavy chains in an antibody. An IgG1 CH3 region typically consists of residues 341-446 on IgG1 (residue numbering according to the EU index). In some embodiments described herein, the bispecific PSMAxCD3 antibody comprises a F405L substitution in an antibody first heavy chain (HC1) and a 409R substitution in an antibody second heavy chain (HC2). In some embodiments, the bispecific PSMAxCD3 antibody comprises a S228P substitution in the HC1 and S228P, F405L and R409K substitutions in the HC2, wherein the antibody is of IgG4 isotype. In some embodiments, the HC1 contains the first domain specifically binding PSMA and the HC2 contains the second domain specifically binding CD3. In some embodiments, the bispecific antibody comprises at least one, two, three, four, five, six, seven or eight asymmetrical substitutions in the HC1 and the HC2 at residue positions 350, 366, 368, 370, 399, 405, 407 or 409, when residue numbering is according to the EU index. In some embodiments, the bispecific antibody comprises at least one, two, three or four asymmetrical substitutions in the HC1 and the HC2 at residue positions 350, 370, 405 or 409, when residue numbering is according to the EU index. In some embodiments, the bispecific antibody comprises at least one asymmetrical substitution in the HC1 and the HC2 at residue positions 405 or 409, when residue numbering is according to the EU index. In some embodiments, the bispecific antibody comprises a 409R or a F405L substitution in the HC1 and a 409R or a F405L substitution in the HC2, wherein residue numbering is according to the EU index. In some embodiments, the bispecific antibody comprises the F405L substitution in the HC1 and the 409R substitution in the HC2. In some embodiments, the bispecific antibody comprises at least one asymmetrical substitution in the HC1 and the HC2 at residue positions 366, 368, 370, 399, 405, 407 or 409, wherein residue numbering is according to the EU index. In some embodiments described herein, the HC1 position 409 has an amino acid substitution other than Lys, Leu or Met and the HC2 position 405 has an amino acid substitution other than Phe. In some embodiments described herein, the HC1 position 405 has an amino acid substitution other than Phe and the HC2 position 409 has an amino acid substitution other than Lys, Leu or Met. In some embodiments described herein, the HC1 position 409 has an amino acid substitution other than Lys, Leu or Met and the HC2 position 405 has an amino acid substitution other than Phe, Arg or Gly. In some embodiments described herein, the HC1 position 405 has an amino acid substitution other than Phe, Arg or Gly and the HC2 CH3 position 409 has an amino acid substitution other than Lys, Leu or Met. In some embodiments described herein, the HC1 CH3 has Phe at position 405 and an amino acid other than Lys, Leu or Met at position 409 and the HC2 has an amino acid other than Phe at position 405 and a Lys at position 409. In some embodiments described herein, the HC1 has an amino acid other than Phe at position 405 and Lys at position 409 and the HC2 has Phe at position 405 and an amino acid other than Lys, Leu or Met at position 409. In some embodiments described herein, the HC1 has Phe at position 405 and an amino acid other than Lys, Leu or Met at position 409 and the HC2 has a substitution other than Phe, Arg or Gly at position 405 and Lys at position 409. In some embodiments described herein, the HC1 has a substitution other than Phe, Arg or Gly at position 405 and Lys at position 409 and the HC2 has Phe at position 405 and an amino acid other than Lys, Leu or Met at position 409. In some embodiments described herein, the HC1 has Phe at position 405 and an amino acid other than Lys, Leu or Met at position 409 and the HC2 has Leu at position 405 and Lys at position 409. In some embodiments described herein, the HC1 has Leu at position 405 and Lys at position 409 and the HC2 has Phe at position 405 and an amino acid other than Lys, Leu or Met at position 409. In some embodiments described herein, the HC1 has Phe at position 405 and Arg at position 409 and the HC2 has an amino acid other than Phe, Arg or Gly at position 405 and Lys at position 409. In some embodiments described herein, the HC1 has an amino acid other than Phe, Arg or Gly at position 405 and Lys at position 409 and the HC2 has Phe at position 405 and Arg at position 409. In some embodiments described herein, the HC1 has Phe at position 405 and Arg at position 409 and the HC2 has Leu at position 405 and Lys at position 409. In some embodiments described herein, the HC1 has Leu at position 405 and Lys at position 409 and the HC2 has Phe at position 405 and Arg at position 409. In some embodiments described herein, the HC1 has Phe at position 405 and Lys at position 409 and the HC2 has Leu at position 405 and Arg at position 409. In some embodiments described herein, the HC1 has Leu at position 405 and Arg at position 409 and the HC2 has Phe at position 405 and Lys at position 409. In some embodiments described herein, the HC1 has an amino acid other than Lys, Leu or Met at position 409 and the HC2 has Lys at position 409, Thr at position 370 and Leu at position 405. In some embodiments described herein, the HC1 has Lys at position 409, Thr at position 370 and Leu at position 405 and the HC2 has an amino acid other than Lys, Leu or Met at position 409. In some embodiments described herein, the HC1 has Arg at position 409 and the HC2 has Lys at position 409, Thr at position 370 and Leu at position 405. In some embodiments described herein, the HC1 has Lys at position 409, Thr at position 370 and Leu at position 405 and the HC2 has Arg at position 409. In some embodiments described herein, the HC1 has Lys at position 370, Phe at position 405 and Arg at position 409 and the HC2 has Lys at position 409, Thr at position 370 and Leu at position 405. In some embodiments described herein, the HC1 has Lys at position 409, Thr at position 370 and Leu at position 405 and the HC2 has Lys at position 370, Phe at position 405 and Arg at position 409. In some embodiments described herein, the HC1 has an amino acid other than Lys, Leu or Met at position 409 and the HC2 has an amino acid other than Tyr, Asp, Glu, Phe, Lys, Gln, Arg, Ser or Thr at position 407. In some embodiments described herein, the HC1 has an amino acid other than Tyr, Asp, Glu, Phe, Lys, Gln, Arg, Ser or Thr at position 407 and the HC2 has an amino acid other than Lys, Leu or Met at position 409. In some embodiments described herein, the HC1 has an amino acid other than Lys, Leu or Met at position 409 and the HC2 has Ala, Gly, His, Ile, Leu, Met, Asn, Val or Trp at position 407. In some embodiments described herein, the HC1 has Ala, Gly, His, Ile, Leu, Met, Asn, Val or Trp at position 407 and the HC2 has an amino acid other than Lys, Leu or Met at position 409. In some embodiments described herein, the HC1 has an amino acid other than Lys, Leu or Met at position 409 and the HC2 has Gly, Leu, Met, Asn or Trp at position 407. In some embodiments described herein, the HC1 has Gly, Leu, Met, Asn or Trp at position 407 and the HC2 has an amino acid other than Lys, Leu or Met at position 409. In some embodiments described herein, the HC1 has Tyr at position 407 and an amino acid other than Lys, Leu or Met at position 409 and the HC2 has an amino acid other than Tyr, Asp, Glu, Phe, Lys, Gln, Arg, Ser or Thr at position 407 and Lys at position 409. In some embodiments described herein, the HC1 has an amino acid other than Tyr, Asp, Glu, Phe, Lys, Gln, Arg, Ser or Thr at position 407 and Lys at position 409 and the HC2 has Tyr at position 407 and an amino acid other than Lys, Leu or Met at position 409. In some embodiments described herein, the HC1 has Tyr at position 407 and an amino acid other than Lys, Leu or Met at position 409 and the HC2 has Ala, Gly, His, Ile, Leu, Met, Asn, Val or Trp at position 407 and Lys at position 409. In some embodiments described herein, the HC1 has Ala, Gly, His, Ile, Leu, Met, Asn, Val or Trp at position 407 and Lys at position 409 and the HC2 CH3 has Tyr at position 407 and an amino acid other than Lys, Leu or Met at position 409. In some embodiments described herein, the HC1 CH3 has Tyr at position 407 and an amino acid other than Lys, Leu or Met at position 409 and the HC2 has Gly, Leu, Met, Asn or Trp at position 407 and Lys at position 409. In some embodiments described herein, the HC1 has Gly, Leu, Met, Asn or Trp at position 407 and Lys at position 409 and the HC2 has Tyr at position 407 and an amino acid other than Lys, Leu or Met at position 409. In some embodiments described herein, the HC1 has Tyr at position 407 and Arg at position 409 and the HC2 has an amino acid other than Tyr, Asp, Glu, Phe, Lys, Gln, Arg, Ser or Thr at position 407 and Lys at position 409. In some embodiments described herein, the HC1 has an amino acid other than Tyr, Asp, Glu, Phe, Lys, Gln, Arg, Ser or Thr at position 407 and Lys at position 409 and the HC2 has Tyr at position 407 and Arg at position 409. In some embodiments described herein, the HC1 has Tyr at position 407 and Arg at position 409 and the HC2 has Ala, Gly, His, Ile, Leu, Met, Asn, Val or Trp at position 407 and Lys at position 409. In some embodiments described herein, the HC1 has Ala, Gly, His, Ile, Leu, Met, Asn, Val or Trp at position 407 and Lys at position 409 and the HC2 has Tyr at position 407 and Arg at position 409. In some embodiments described herein, the HC1 CH3 has Tyr at position 407 and Arg at position 409 and the HC2 CH3 has Gly, Leu, Met, Asn or Trp at position 407 and Lys at position 409. In some embodiments described herein, the HC1 has Gly, Leu, Met, Asn or Trp at position 407 and Lys at position 409 and the HC2 has Tyr at position 407 and Arg at position 409. In some embodiments described herein, the HC1 has an amino acid other than Lys, Leu or Met at position 409, and the HC2 has (i) an amino acid other than Phe, Leu and Met at position 368, or (ii) a Trp at position 370, or (iii) an amino acid other than Asp, Cys, Pro, Glu or Gln at position 399. In some embodiments described herein, the HC1 has (i) an amino acid other than Phe, Leu and Met at position 368, or (ii) a Trp at position 370, or (iii) an amino acid other than Asp, Cys, Pro, Glu or Gln at position 399 and the HC2 has an amino acid other than Lys, Leu or Met at position 409. In some embodiments described herein, the HC1 has Arg, Ala, His or Gly at position 409, and the HC2 has (i) Lys, Gln, Ala, Asp, Glu, Gly, His, Ile, Asn, Arg, Ser, Thr, Val, or Trp at position 368, or (ii) Trp at position 370, or (iii) Ala, Gly, Ile, Leu, Met, Asn, Ser, Thr, Trp, Phe, His, Lys, Arg or Tyr at position 399. In some embodiments described herein, the HC1 has (i) Lys, Gln, Ala, Asp, Glu, Gly, His, Ile, Asn, Arg, Ser, Thr, Val, or Trp at position 368, or (ii) Trp at position 370, or (iii) Ala, Gly, Ile, Leu, Met, Asn, Ser, Thr, Trp, Phe, His, Lys, Arg or Tyr at position 399 and the HC2 has Arg, Ala, His or Gly at position 409. In some embodiments described herein, the HC1 has Arg at position 409, and the HC2 has (i) Asp, Glu, Gly, Asn, Arg, Ser, Thr, Val, or Trp at position 368, or (ii) Trp at position 370, or (iii) Phe, His, Lys, Arg or Tyr at position 399. In some embodiments described herein, the HC1 has (i) Asp, Glu, Gly, Asn, Arg, Ser, Thr, Val, or Trp at position 368, or (ii) Trp at position 370, or (iii) Phe, His, Lys, Arg or Tyr at position 399 and the HC2 has Arg at position 409. In some embodiments described herein, the HC1 comprises a 409R substitution or a F405L substitution and the HC2 comprises a 409R substitution or a F405L substitution, wherein residue numbering is according to the EU index. In some embodiments described herein, the HC1 comprises the F405L substitution and the HC2 comprises the 409R substitution.

Substitutions are typically made at the DNA level to a molecule such as the constant domain of the antibody using standard methods.

The antibodies provided herein can be engineered into various well-known antibody forms.

In some embodiments, the bispecific antibody is a diabody or a cross-body.

In some embodiments, the multispecific antibodies include IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12: 276 and in Nunez-Prado et al., (2015) Drug Discovery Today 20(5):588-594.

Any of the VH and the VL domains identified herein (e.g., those that bind PSMA) can be engineered into scFv format. In some embodiments, the scFv format is in the VH-linker-VL orientation. In other embodiments, the scFv format is in the VL-linker-VH orientation. Any of the VH and the VL domains identified herein can also be used to generate sc(Fv)₂ structures. In some embodiments, the sc(Fv)₂ structure is VH-linker-VL-linker-VL-linker-VH. In some embodiments, the sc(Fv)₂ structure is VH-linker-VL-linker-VH-linker-VL. In some embodiments, the sc(Fv)₂ structure is VH-linker-VH-linker-VL-linker-VL. In some embodiments, the sc(Fv)₂ structure is VL-linker-VH-linker-VH-linker-VL. In some embodiments, the sc(Fv)₂ structure is VL-linker-VH-linker-VL-linker-VH. In some embodiments, the sc(Fv)₂ structure is VL-linker-VL-linker-VH-linker-VH.

In specific embodiments, the linker is a peptide linker. In some embodiments, the liker comprises a naturally occurring amino acid. Exemplary amino acids that can be included into the linker are Gly, Ser Pro, Thr, Glu, Lys, Arg, Ile, Leu, His and The. In some embodiments, the linker has a length that is adequate to link the VH and the VL in such a way that they form the correct conformation relative to one another so that they retain the desired activity, such as binding to the target (e.g., PSMA).

In certain embodiments, the linker is about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that can be used are Gly rich linkers, Gly and Ser containing linkers, Gly and Ala containing linkers, Ala and Ser containing linkers, and other flexible linkers.

Exemplary linkers that can be used include any one of SEQ ID NOs. 1419-1452. Exemplary linkers are shown in Table 2. Additional linkers are described for example in Int. Pat. Publ. No. WO2019/060695. In some embodiments, the linker comprises or consists of the amino acid sequence of SEQ ID NO:1419. In some embodiments, the linker comprises or consists of the amino acid sequence of SEQ ID NO:1420. In some embodiments, the linker comprises or consists of the amino acid sequence of SEQ ID NO:1421. In some embodiments, the linker comprises or consists of the amino acid sequence of SEQ ID NO:1422. In some embodiments, the linker comprises or consists of the amino acid sequence of SEQ ID NO:1423. In some embodiments, the linker comprises or consists of the amino acid sequence of SEQ ID NO:1424. In some embodiments, the linker comprises or consists of the amino acid sequence of SEQ ID NO:1425. In some embodiments, the linker comprises or consists of the amino acid sequence of SEQ ID NO:1426. In some embodiments, the linker comprises or consists of the amino acid sequence of SEQ ID NO:1427. In some embodiments, the linker comprises or consists of the amino acid sequence of SEQ ID NO:1428. In some embodiments, the linker comprises or consists of the amino acid sequence of SEQ ID NO:1429. In some embodiments, the linker comprises or consists of the amino acid sequence of SEQ ID NO:1430. In some embodiments, the linker comprises or consists of the amino acid sequence of SEQ ID NO:1431. In some embodiments, the linker comprises or consists of the amino acid sequence of SEQ ID NO:1432. In some embodiments, the linker comprises or consists of the amino acid sequence of SEQ ID NO:1433. In some embodiments, the linker comprises or consists of the amino acid sequence of SEQ ID NO:1434. In some embodiments, the linker comprises or consists of the amino acid sequence of SEQ ID NO:1435. In some embodiments, the linker comprises or consists of the amino acid sequence of SEQ ID NO:1436. In some embodiments, the linker comprises or consists of the amino acid sequence of SEQ ID NO:1437. In some embodiments, the linker comprises or consists of the amino acid sequence of SEQ ID NO:1438. In some embodiments, the linker comprises or consists of the amino acid sequence of SEQ ID NO:1439. In some embodiments, the linker comprises or consists of the amino acid sequence of SEQ ID NO:1440. In some embodiments, the linker comprises or consists of the amino acid sequence of SEQ ID NO:1441. In some embodiments, the linker comprises or consists of the amino acid sequence of SEQ ID NO:1442. In some embodiments, the linker comprises or consists of the amino acid sequence of SEQ ID NO:1427. In some embodiments, the linker comprises or consists of the amino acid sequence of SEQ ID NO:1444. In some embodiments, the linker comprises or consists of the amino acid sequence of SEQ ID NO:1445. In some embodiments, the linker comprises or consists of the amino acid sequence of SEQ ID NO:1446. In some embodiments, the linker comprises or consists of the amino acid sequence of SEQ ID NO:1435. In some embodiments, the linker comprises or consists of the amino acid sequence of SEQ ID NO:1448. In some embodiments, the linker comprises or consists of the amino acid sequence of SEQ ID NO:1449. In some embodiments, the linker comprises or consists of the amino acid sequence of SEQ ID NO:1450. In some embodiments, the linker comprises or consists of the amino acid sequence of SEQ ID NO:1451. In some embodiments, the linker comprises or consists of the amino acid sequence of SEQ ID NO:1452.

TABLE 2
Exemplary Linkers

| | | SEQ |
|---|---|---|
| Linker | | ID |
| name | Amino acid sequence | NO: |
| Linker 1 | GGSEGKSSGSGSESKSTGGS | 1419 |
| Linker 2 | GGGSGGGS | 1420 |
| Linker 3 | GGGSGGGSGGGS | 1421 |
| Linker 4 | GGGSGGGSGGGSGGGS | 1422 |
| Linker 5 | GGGSGGGSGGGSGGGSGGGS | 1423 |
| Linker 6 | GGGGSGGGGSGGGGS | 1424 |
| Linker 7 | GGGGSGGGGSGGGGSGGGGS | 1425 |
| Linker 8 | GGGGSGGGGSGGGGSGGGGSGGGGS | 1426 |
| Linker 9 | GSTSGSGKPGSGEGSTKG | 1427 |
| Linker 10 | IRPRAIGGSKPRVA | 1428 |
| Linker 11 | GKGGSGKGGSGKGGS | 1429 |
| Linker 12 | GGKGSGGKGSGGKGS | 1430 |
| Linker 13 | GGGKSGGGKSGGGKS | 1431 |
| Linker 14 | GKGKSGKGKSGKGKS | 1432 |
| Linker 15 | GGGKSGGKGSGKGGS | 1433 |
| Linker 16 | GKPGSGKPGSGKPGS | 1434 |
| Linker 17 | GKPGSGKPGSGKPGSGKPGS | 1435 |
| Linker 18 | GKGKSGKGKSGKGKSGKGKS | 1436 |
| Linker 19 | STAGDTHLGGEDFD | 1437 |
| Linker 20 | GEGGSGEGGSGEGGS | 1438 |
| Linker 21 | GGEGSGGEGSGGEGS | 1439 |
| Linker 22 | GEGESGEGESGEGES | 1440 |
| Linker 23 | GGGESGGEGSGEGGS | 1441 |
| Linker 24 | GEGESGEGESGEGESGEGES | 1442 |
| Linker 25 | GSTSGSGKPGSGEGSTKG | 1427 |
| Linker 26 | PRGASKSGSASQTGSAPGS | 1444 |
| Linker 27 | GTAAAGAGAAGGAAAGAAG | 1445 |
| Linker 28 | GTSGSSGSGSGGSGSGGGG | 1446 |
| Linker 29 | GKPGSGKPGSGKPGSGKPGS | 1435 |
| Linker 30 | GSGS | 1448 |
| Linker 31 | APAPAPAPAP | 1449 |
| Linker 32 | APAPAPAPAPAPAPAPAPAP | 1450 |
| Linker 33 | AEAAAKEAAAKEAAAAKEAAA | 1451 |
| | AKEAAAAKAAA | |
| Linker 34 | GTEGKSSGSGSESKST | 1452 |

In some embodiments, the scFv comprises, from the N- to C-terminus, a VH, a first linker (L1) and a VL (VH-L1-VL). In other embodiments, the scFv comprises, from the N- to C-terminus, the VL, the L1 and the VH (VL-L1-VH). In another embodiment, the L1 comprises the amino acid sequence of SEQ ID NO:1419. In one embodiment, the L1 comprises the amino acid sequence of SEQ ID NO:1420. In other embodiments, the L1 comprises the amino acid sequence of SEQ ID NO:1421. In some embodiments, the L1 comprises the amino acid sequence of SEQ ID NO:1422. In one embodiment, the L1 comprises the amino acid sequence of SEQ ID NO:1423. In some embodiments, the L1 comprises the amino acid sequence of SEQ ID NO:1424. In other embodiments, the L1 comprises the amino acid sequence of SEQ ID NO:1425. In one embodiment, the L1 comprises the amino acid sequence of SEQ ID NO:1426. In some embodiments, the L1 comprises the amino acid sequence of SEQ ID NO:1427. In another embodiment, the L1 comprises the amino acid sequence of SEQ ID NO:1428. In one embodiment, the L1 comprises the amino acid sequence of SEQ ID NO:1429. In other embodiments, the L1 comprises the amino acid sequence of SEQ ID NO:1430. In some embodiments, the L1 comprises the amino acid sequence of SEQ ID NO:1431. In some embodiments, the L1 comprises the amino acid sequence of SEQ ID NO:1432. In one embodiment, the L1 comprises the amino acid sequence of SEQ ID NO:1433. In other embodiments, the L1 comprises the amino acid sequence of SEQ ID NO:1434. In one embodiment, the L1 comprises the amino acid sequence of SEQ ID NO:1435. In another embodiment, the L1 comprises the amino acid sequence of SEQ ID NO:1436. In some embodiments, the L1 comprises the amino acid sequence of SEQ ID NO:1437. In one embodiment, the L1 comprises the amino acid sequence of SEQ ID NO:1438. In other embodiments, the L1 comprises the amino acid sequence of SEQ ID NO:1439. In some embodiments, the L1 comprises the amino acid sequence of SEQ ID NO:1440. In another embodiment, the L1 comprises the amino acid sequence of SEQ ID NO:1441. In one embodiment, the L1 comprises the amino acid sequence of SEQ ID NO:1442. In other embodiments, the L1 comprises the amino acid sequence of SEQ ID NO:1427. In one embodiment, the L1 comprises the amino acid sequence of SEQ ID NO:1444. In some embodiments, the L1 comprises the amino acid sequence of SEQ ID NO:1445. In other embodiments, the L1 comprises the amino acid sequence of SEQ ID NO:1446. In one embodiment, the L1 comprises the amino acid sequence of SEQ ID NO:1435. In another embodiment, the L1 comprises the amino acid sequence of SEQ ID NO:1448. In one embodiment, the L1 comprises the amino acid sequence of SEQ ID NO:1449. In other embodiments, the L1 comprises the amino acid sequence of SEQ ID NO:1450. In another embodiment, the L1 comprises the amino acid sequence of SEQ ID NO:1451. In one embodiment, the L1 comprises the amino acid sequence of SEQ ID NO:1452.

In certain embodiments, antibodies, including provided herein comprise two linkers. In other embodiments antibodies provided herein comprise three linkers. In yet other embodiments, antibodies provided herein comprise four or more linkers. In certain embodiments, the antibody is an antigen binding fragment thereof.

### Polynucleotides, Vectors and Host Cells

Also provided is a nucleic acid encoding an antibody provided herein. In another general aspect, provide is a vector comprising an isolated nucleic acid encoding an antibody provided herein. In another general aspect, provided is a vector comprising an isolated nucleic acid encoding an antibody provided herein. Also provided is a vector comprising a nucleic acid encoding an antibody provided herein. Also provided is a host cell comprising a vector comprising a nucleic acid encoding an antibody provided herein. Also provided is a kit comprising the vector comprising a nucleic acid encoding an antibody provided herein, and packaging for the same. In another general aspect, provided herein is an isolated nucleic acid encoding a monoclonal antibody or antigen-binding fragment thereof provided herein. In certain embodiments, the antibody is a PSMA antibody. In certain embodiments, the antibody is a multispecific PSMA antibody. In some embodiments, the antibody is a multispecific PSMAxCD3 antibody. Also provided is a nucleic acid encoding a bispecific antibody comprising: (a) a first binding domain that binds to PSMA, and (b) a second binding domain that binds to a second target that is not PSMA, as provided herein.

Also provided is a nucleic acid encoding a multispecific PSMAxCD3 antibody provided herein. In another general aspect, provide is a vector comprising an isolated nucleic acid encoding a multispecific PSMAxCD3 antibody provided herein. In another general aspect, provided is a vector comprising an isolated nucleic acid encoding a multispecific PSMAxCD3 antibody provided herein. Also provided is a vector comprising a nucleic acid encoding a multispecific PSMAxCD3 antibody provided herein. Also provided is a host cell comprising a vector comprising a nucleic acid encoding a multispecific PSMAxCD3 antibody provided herein. Also provided is a kit comprising the vector comprising a nucleic acid encoding a multispecific PSMAxCD3 antibody provided herein, and packaging for the same. Also provided is an isolated nucleic acid encoding a monoclonal antibody provided herein. Also provided is an isolated nucleic acid encoding an antigen binding fragment provided herein. Also provided is a nucleic acid encoding a multispecific antibody comprising: (a) a first binding domain that binds to PSMA, and (b) a second binding domain that binds to a CD3, as provided herein. In certain embodiments, the multispecific PSMAxCD3 antibody is a trispecific antibody. In certain embodiments, the multispecific PSMAxCD3 antibody is a quadraspecific antibody.

Also disclosed are isolated polynucleotides that encode the antibodies or antigen-binding fragments that immunospecifically bind to PSMA. The isolated polynucleotides capable of encoding the variable domain segments provided herein can be included on the same, or different, vectors to produce antibodies or antigen-binding fragments.

Polynucleotides encoding recombinant antigen-binding proteins also are within the scope of the disclosure. In some embodiments, the polynucleotides described (and the peptides they encode) include a leader sequence. Any leader sequence known in the art can be employed. The leader sequence can include, but is not limited to, a restriction site or a translation start site.

The PSMA-specific antibodies or antigen-binding fragments described herein include variants having single or multiple amino acid substitutions, deletions, or additions that retain the biological properties (e.g., binding affinity or immune effector activity) of the described PSMA-specific antibodies or antigen-binding fragments. In certain embodiments, the following notations are, unless otherwise indicated, used to describe a mutation; i) substitution of an amino acid in a given position is written as e.g. K409R which means a substitution of a Lysine in position 409 with an Arginine; and ii) for specific variants the specific three or one letter codes are used, including the codes Xaa and X to indicate any amino acid residue. Thus, the substitution of Arginine for Lysine in position 409 is designated as: K409R, or the substitution of any amino acid residue for Lysine in position 409 is designated as K409X. In case of deletion of Lysine in position 409 it is indicated by K409*. Where a particular amino acid residue may vary among peptide isotypes or variants, and a substitution affects that residue in each isotype or variant or any of the isotypes or variants, the substitution is designated as, e.g., 409R, meaning the amino acid corresponding to position 409 is substituted with Arginine. The skilled person can produce variants having single or multiple amino acid substitutions, deletions, or additions.

In some embodiments, these variants are: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that can confer useful properties to the polypeptide, such as, for example, an epitope for an antibody, a polyhistidine sequence, a biotin moiety and the like. Antibodies or antigen-binding fragments described herein can include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (deletions, mutations, etc.), chemical, and enzymatic techniques, are known to persons having ordinary skill in the art.

The PSMA-specific antibodies or antigen-binding fragments described herein can embody several antibody isotypes, such as IgM, IgD, IgG, IgA and IgE. In some embodiments the antibody isotype is IgG1, IgG2, IgG3, or IgG4 isotype, such as IgG1 or IgG4 isotype. Antibody or antigen-binding fragment thereof specificity is largely determined by the amino acid sequence, and arrangement, of the CDRs. Therefore, the CDRs of one isotype can be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antibodies provided herein can be altered without changing the amino acid sequences of the proteins.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to certain embodiments. Such techniques are well known to those skilled in the art in view of the present disclosure.

Also provided, are vectors comprising the polynucleotides described herein. The vectors can be expression vectors. Recombinant expression vectors containing a sequence encoding a polypeptide of interest are thus contemplated as within the scope of this disclosure. The expression vector can contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

Recombinant expression vectors within the scope of the description include synthetic, genomic, or cDNA-derived nucleic acid fragments that encode at least one recombinant protein which can be operably linked to suitable regulatory elements. Such regulatory elements can include a transcriptional promoter, sequences encoding suitable mRNA ribosomal binding sites, and sequences that control the termination of transcription and translation. Expression vectors, especially mammalian expression vectors, can also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host can also be incorporated.

The transcriptional and translational control sequences in expression vectors to be used in transforming vertebrate cells can be provided by viral sources. Exemplary vectors can be constructed as described by Okayama and Berg, 3 Mol. Cell. Biol. 280 (1983).

In some embodiments, the antibody- or antigen-binding fragment-coding sequence is placed under control of a powerful constitutive promoter, such as the promoters for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the PSMA-specific antibody or antigen-binding fragment thereof coding sequence is placed under control of an inducible promoter such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like.

Vectors described herein can contain one or more Internal Ribosome Entry Site(s) (IRES). Inclusion of an IRES sequence into fusion vectors can be beneficial for enhancing expression of some proteins. In some embodiments the vector system will include one or more polyadenylation sites (e.g., SV40), which can be upstream or downstream of any of the aforementioned nucleic acid sequences. Vector components can be contiguously linked, or arranged in a manner that provides optimal spacing for expressing the gene products (i.e., by the introduction of “spacer” nucleotides between the ORFs), or positioned in another way. Regulatory elements, such as the IRES motif, can also be arranged to provide optimal spacing for expression.

The vectors can comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site can be upstream or downstream of a nucleic acid sequence encoding a polypeptide of interest or cloning site.

The vectors described herein can be used to transform various cells with the genes encoding the described antibodies or antigen-binding fragments. For example, the vectors can be used to generate PSMA-specific antibody or antigen-binding fragment-producing cells. Thus, another aspect features host cells transformed with vectors comprising a nucleic acid sequence encoding an antibody or antigen-binding fragment thereof that specifically binds PSMA, such as the antibodies or antigen-binding fragments described and exemplified herein.

Numerous techniques are known in the art for the introduction of foreign genes into cells and can be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which can be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells can also be used to transform cells.

Also provided is a host cell comprising an isolated nucleic acid encoding an antibody provided herein. Also provided is a host cell comprising an isolated nucleic acid encoding an antigen binding fragment provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). Other cells suitable for use in the expression of the PSMA-specific antibodies or antigen-binding fragments described herein can be eukaryotic cells, such as cells of plant, rodent, or human origin, for example but not limited to NSO, CHO, CHOK1, perC.6, Tk-ts13, BHK, HEK293 cells, COS-7, T98G, CV-1/EBNA, L cells, C127, 3T3, HeLa, NS1, Sp2/0 myeloma cells, and BHK cell lines, among others. In addition, expression of antibodies can be accomplished using hybridoma cells. Methods for producing hybridomas are well established in the art.

According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Cells transformed with expression vectors described herein can be selected or screened for recombinant expression of the antibodies or antigen-binding fragments described herein. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes can be due to inherent properties of a given subclone or to mutation. Mutations can be effected through the use of chemicals, UV-wavelength light, radiation, viruses, insertional mutagens, inhibition of DNA mismatch repair, or a combination of such methods.

Also provided are methods of producing an antibody disclosed herein. The methods comprise culturing a cell comprising a nucleic acid encoding the antibody under conditions to produce an antibody and recovering the antibody from the cell or cell culture (e.g., from the supernatant). Expressed antibodies can be harvested from the cells and purified according to conventional techniques known in the art and as described herein.

Also provided is a method of producing a multispecific antibody disclosed herein. The methods comprise culturing a cell comprising a nucleic acid encoding the multispecific antibody thereof under conditions to produce the multispecific antibody and recovering the multispecific antibody from the cell or cell culture (e.g., from the supernatant). Expressed multispecific antibodies thereof can be harvested from the cells and purified according to conventional techniques known in the art and as described herein. Other methods are described elsewhere herein.

### Pharmaceutical Compositions

In another general aspect, provided is a pharmaceutical composition comprising a PSMA antibody provided herein and a pharmaceutically acceptable carrier. In certain embodiments, the antibody is isolated. Also provided is a method of producing the pharmaceutical composition, comprising combining the antibody with a pharmaceutically acceptable carrier to obtain the pharmaceutical composition.

In another general aspect, provided is a pharmaceutical composition comprising a PSMA multispecific antibody provided herein and a pharmaceutically acceptable carrier. In certain embodiments, the multispecific antibody is isolated. Also provided is a method of producing the pharmaceutical composition, comprising combining the multispecific antibody with a pharmaceutically acceptable carrier to obtain the pharmaceutical composition. In another aspect, provided herein is a pharmaceutical composition comprising a comprising: (a) a first binding domain that binds to PSMA, and (b) a second binding domain that binds to a second target, and a pharmaceutically acceptable carrier. Any of the multispecific antibodies provided herein are contemplated in the pharmaceutical compositions. In certain embodiments, the second binding domain binds to CD3. Any of the antibodies provided herein are contemplated in the pharmaceutical compositions.

In another general aspect, provided is a pharmaceutical composition comprising a multispecific PSMAxCD3 antibody provided herein and a pharmaceutically acceptable carrier. In certain embodiments, the multispecific PSMAxCD3 antibody is isolated. Also provided is a method of producing the pharmaceutical composition, comprising combining the multispecific antibody with a pharmaceutically acceptable carrier to obtain the pharmaceutical composition. In another aspect, provided herein is a pharmaceutical composition comprising a comprising: (a) a first binding domain that binds to PSMA, and (b) a second binding domain that binds to CD3, and a pharmaceutically acceptable carrier. Any of the multispecific antibodies provided herein are contemplated in the pharmaceutical compositions.

The term “pharmaceutical composition” as used herein means a product comprising an antibody provided herein together with a pharmaceutically acceptable carrier. Antibodies of provided herein and compositions comprising them are also useful in the manufacture of a medicament for therapeutic applications.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition provided herein the biological activity of a composition provided herein. According to particular embodiments, in view of the present disclosure, any pharmaceutically acceptable carrier suitable for use in an antibody pharmaceutical composition can be used herein. Carrier can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They can be sterilized by conventional, well-known sterilization techniques (e.g., filtration). The compositions can contain pharmaceutically acceptable auxiliary substances as required to approximate physiological conditions such as pH adjusting and buffering agents, stabilizing, thickening, lubricating and coloring agents, etc. The concentration of the antibodies provided herein in such pharmaceutical formulation can vary, from less than about 0.5%, usually to at least about 1% to as much as 15 or 20% by weight and can be selected primarily based on required dose, fluid volumes, viscosities, etc., according to the particular mode of administration selected. Suitable vehicles and formulations, inclusive of other human proteins, e.g., human serum albumin. The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions provided herein.

In one embodiment, the pharmaceutical composition is a liquid formulation. An example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% wxw water, or at least 60%, 70%, 75%, 80%, 85%, 90%, or at least 95% wxw of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment, the pH of the formulation is from about 7.0 to about 9.5. In another embodiment, the pH of the formulation is from about 3.0 to about 7.0.

In another embodiment, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mgxml to about 50 mgxml, for example from about 0.1 mgxml to about 20 mgxml. Pharmaceutical compositions comprising each one of these specific buffers constitute alternative embodiments.

In another embodiment, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mgxml to about 50 mgxml, for example from about 0.1 mgxml to about 20 mgxml. Pharmaceutical compositions comprising each one of these specific preservatives constitute alternative embodiments.

In another embodiment, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can include mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethyl-cellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mgxml to about 50 mgxml, for example from about 0.1 mgxml to about 20 mgxml. Pharmaceutical compositions comprising each one of these specific isotonic agents constitute alternative embodiments.

In another embodiment, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mgxml to about 50 mgxml, for example from about 0.1 mgxml to about 20 mgxml. Pharmaceutical compositions comprising each one of these specific chelating agents constitute alternative embodiments.

In another embodiment, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment, the pharmaceutical composition comprises a stabilizer, wherein said stabilizer is carboxy-xhydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mgxml to about 50 mgxml, for example from about 0.1 mgxml to about 20 mgxml. Pharmaceutical compositions comprising each one of these specific stabilizers constitute alternative embodiments.

In further embodiments, the pharmaceutical composition comprises one or more surfactants, such as a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mgxml to about 20 mgxml. Pharmaceutical compositions comprising each one of these specific surfactants constitute alternative embodiments.

In a further embodiment, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mgxml to about 20 mgxml. Pharmaceutical compositions comprising each one of these specific protease inhibitors constitute alternative embodiments.

In another general aspect, provided herein is a method of producing a pharmaceutical composition comprising an antibody or antigen-binding fragment thereof provided herein, comprising combining an antibody or antigen-binding fragment thereof with a pharmaceutically acceptable carrier to obtain the pharmaceutical composition.

### Methods of Administration

The mode of administration for therapeutic use of the antibodies provided herein can be any suitable route that delivers the antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration can be achieved by for example intratumoral, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

The antibodies provided herein can be administered to a subject by any suitable route, for example parentally by intravenous (i.v.) infusion or bolus injection, intramuscularly or subcutaneously or intraperitoneally. i.v. infusion can be given over for example 15, 30, 60, 90, 120, 180, or 240 minutes, or from 1, 2, 3, 4, 5, 6, 7, 8, 9, 10, 11 or 12 hours.

The dose given to a subject is sufficient to alleviate or at least partially arrest the disease being treated (“therapeutically effective amount”) and can be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but can even higher, for example about 15, 16, 17, 18, 19, 20, 21, 22, 23, 24, 25, 30, 40, 50, 60, 70, 80, 90 or 100 mg/kg.

A fixed unit dose can also be given, for example, 50, 100, 200, 500 or 1000 mg, or the dose can be based on the patient's surface area, e.g., 500, 400, 300, 250, 200, or 100 mg/m2. Usually between 1 and 8 doses, (e.g., 1, 2, 3, 4, 5, 6, 7 or 8) can be administered to treat the patient, but 9, 10, 11, 12, 13, 14, 15, 16, 17, 18, 19, 20 or more doses can be given.

The administration of the antibodies of provided herein, and in some embodiments of each and every one of the numbered embodiments listed below, can be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration can be at the same dose or at a different dose. For example, the antibodies described herein can be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

For example, the antibodies in the methods described herein, and in some embodiments of each and every one of the numbered embodiments listed below, can be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The antibodies in the methods described herein, and in some embodiments of each and every one of the numbered embodiments listed below, can also be administered prophylactically in order to reduce the risk of developing cancer, delay the onset of the occurrence of an event in cancer progression, and/or reduce the risk of recurrence when a cancer is in remission.

The antibodies provided herein can be lyophilized for storage and reconstituted in a suitable carrier prior to use. This technique has been shown to be effective with conventional protein preparations and well known lyophilization and reconstitution techniques can be employed.

### Methods of Use

The functional activity of antibodies provided herein can be characterized by methods known in the art and as described herein. Methods for characterizing antibodies and antigen-binding fragments thereof include, but are not limited to, affinity and specificity assays including Biacore, ELISA, and OCTETRED analysis; binding assays to detect the binding of antibodies to target cells by FACS; binding assays to detect the binding of antibodies to the target antigen on cells. According to particular embodiments, the methods for characterizing antibodies and antigen-binding fragments thereof include those described below. In certain embodiments, the antibody is a PSMA antibody. In some embodiments, the PSMA antibody is a PSMA multispecific antibody provided herein.

Also provided is a method of activating a T cell, comprising contacting the T cell with a multispecific PSMA antibody provided herein, wherein the second binding domain binds the T cell. In another general aspect, provided is a method of inactivating a T cell, comprising contacting the T cell with a multispecific PSMA antibody provided herein, wherein the second binding domain binds the T cell. In another general aspect, provided is a method of blocking activation of a T cell, comprising contacting the T cell with a multispecific PSMA antibody provided herein, wherein the second binding domain binds the T cell. In another general aspect, provided is a method of modulating the activation of a T cell, comprising contacting the T cell with a multispecific antibody provided herein, wherein the second binding domain binds the T cell. In some embodiments the T cell expresses CD3. In some embodiments, the second binding domain binds CD3. In some embodiments, the multispecific PSMA antibody is a multispecific PSMAxCD3 antibody.

In another aspect, provided herein is a method of directing a T cell to a target cell, the method comprising contacting the T cell with a multispecific PSMA antibody provided herein. In another aspect, provided herein is a method of directing a T cell to a target cell, the method comprising contacting the T cell with a pharmaceutical composition comprising a multispecific PSMA antibody provided herein. In some embodiments, the contacting directs the T cell to the target cell. In some embodiments the target cell expresses PSMA. In some embodiments, the target cell is a prostate cell. In some embodiments, the target cell is a prostate cancer cell. In some embodiments, the target cell is a renal cell. In some embodiments, the target cell is a renal cancer cell. In some embodiments the T cell expresses CD3. In some embodiments, the multispecific PSMA antibody is a multispecific PSMAxCD3 antibody.

Also provided is a method of targeting an antigen on the surface of a target cell, the method comprising exposing the target cell to a PSMA antibody provided herein. Also provided is a method of targeting an antigen on the surface of a target cell, the method comprising exposing the target cell to a pharmaceutical composition comprising a PSMA antibody provided herein. In some embodiments the target cell expresses PSMA. In some embodiments, the target cell is a prostate cell. In some embodiments, the target cell is a prostate cancer cell. In some embodiments, the target cell is a renal cell. In some embodiments, the target cell is a renal cancer cell.

In another general aspect, provided is a method of targeting an antigen on the surface of a target cell, the method comprising exposing the target cell to a PSMA multispecific antibody provided herein. In another general aspect, provided is a method of targeting an antigen on the surface of a target cell, the method comprising exposing the target cell to a pharmaceutical composition comprising a PSMA multispecific antibody provided herein. In some embodiments the target cell expresses CD3. In some embodiments, the multispecific PSMA antibody is a multispecific PSMAxCD3 antibody.

PSMA is highly expressed in prostatic intraepithelial neoplasia (PIN), a condition in which some prostate cells have begun to look and behave abnormally, and in primary and metastatic prostate cancers (Bostwick et al. Cancer 1998; 82 (11):2256-2261); and the neovasculature of other solid tumors (e.g., breast, lung, bladder, kidney). Expression of PSMA in cancer tissues correlates with the stage of disease and Gleason score (Kawakami et al. Cancer Res 1997; 57(12):2321-2324). PSMA expression is also higher in prostate cancer cells from hormone-refractory patients (Wright et al. Urology 1996; 48(2):326-334). Increased PSMA expression has been shown to be an independent marker of disease recurrence (Mitsiades et al. Clin Exp Metastasis 2004; 21(6):495-505). High-level PSMA expression is correlated with early prostate-specific antigen (PSA) recurrence in surgically treated prostate cancer. PSMA expression levels correlate with the aggressiveness of the disease. PSMA expression is increased in metastatic disease, hormone refractory cases, and higher-grade lesions, and it is further upregulated in androgen-insensitive tumors. Thus, this target is useful for prostate cancer characterization and subsequent therapy.

Blockade of PSMA can inhibit or decrease the growth of PSMA-expressing cancerous cells and tumors in a subject. It can also have antiangiogenic activity owing to expression of PSMA in tumor neovasculature (Milowsky, et al. 2007). PSMA is highly expressed in prostatic intraepithelial neoplasia, the most established precursor of prostatic carcinoma, and therefore blockade of PSMA can modulate progression of PIN to prostate cancer

In one aspect, provided is a method of inhibiting growth of tumor cells in a subject, comprising administering to the subject a therapeutically effective amount of a PSMA antibody provided herein. In some embodiments, the antibody is a multispecific PSMA antibody. In some embodiments, the multispecific PSMA antibody is a bispecific antibody. In certain embodiments, the multispecific PSMA antibody is a PSMAxCD3 antibody provided herein.

In one aspect, provided is a method of inhibiting the formation or growth of neovasculature of a tumor in a subject, comprising administering to the subject a therapeutically effective amount of a PSMA antibody provided herein. In some embodiments, the antibody is a multispecific PSMA antibody. In some embodiments, the multispecific PSMA antibody is a bispecific antibody. In certain embodiments, the multispecific PSMA antibody is a PSMAxCD3 antibody provided herein.

In one aspect, provided is a method of inhibiting progression of a precancerous state in a subject, comprising administering to the subject a therapeutically effective amount of a PSMA antibody provided herein. In some embodiments, the antibody is a multispecific PSMA antibody. In some embodiments, the multispecific PSMA antibody is a bispecific antibody. In certain embodiments, the multispecific PSMA antibody is a PSMAxCD3 antibody provided herein.

In one aspect, provided is a method of treating a cancer by administering to the subject a PSMA antibody provided herein. In some embodiments, the antibody is a multispecific PSMA antibody. In some embodiments, the multispecific PSMA antibody is a bispecific antibody. In certain embodiments, the multispecific PSMA antibody is a PSMAxCD3 antibody provided herein.

Exemplary antibodies that can be used in the methods provided herein are antibodies specifically binding PSMA and bispecific PSMAxCD3 antibodies as described herein. Exemplary PSMA antibodies can be monospecific. Other exemplary PSMA antibodies can be part of a CD3 bispecific antibody, as provided herein. In certain embodiments, the PSMA antibody is PSMB889. In other embodiments, the PSMA antibody is PSMB890. In certain embodiments, the PSMA antibody is PSMB891. In certain embodiments, the PSMA antibody is PSMB892. In other embodiments, the PSMA antibody is PSMB893. In certain embodiments, the PSMA antibody is PSMB894. In other embodiments, the PSMA antibody is PSMB895. In certain embodiments, the PSMA antibody is PSMB896. In certain embodiments, the PSMA antibody is PSMB897. In other embodiments, the PSMA antibody is PSMB898. In certain embodiments, the PSMA antibody is PSMB899. In certain embodiments, the PSMA antibody is PSMHB49SC1133_011A11_1. In other embodiments, the PSMA antibody is PSMB896-G100A. In certain embodiments, the PSMA antibody is PSMA_P72_A10-HC-G54E. In certain embodiments, the PSMA antibody is PSMA_P72_D01-HC-D95E. In other embodiments, the PSMA antibody is PSMA_P72_F01. In other embodiments, the PSMA antibody is PSMA_P75_F01. In certain embodiments, the PSMA antibody is PSMA_P72_F07. In certain embodiments, the PSMA antibody is PSMA_P72_E07. In other embodiments, the PSMA antibody is PSMA_P72_D01. In certain embodiments, the PSMA antibody is PSMA_P72_C01. In other embodiments, the PSMA antibody is PSMA_P72_A10. In certain embodiments, the PSMA antibody is PSMA_P72_F02. In certain embodiments, the PSMA antibody is PSMA_P70_F02. In other embodiments, the PSMA antibody is PSMA_P72_G02. In other embodiments, the PSMA antibody is PSMA_P72_A11. Each of these antibodies is further described and characterized in the Examples below, including Tables 4-12 or 23-28. In some embodiments, the antibody is a PSMA antibody comprising a VH of any of the PSMA antibodies provided in Tables 4-12 or 23-28. In some embodiments, the antibody is a PSMA antibody comprising a VL of any of the PSMA antibodies provided in Tables 4-12 or 23-28. In some embodiments, the antibody is a PSMA antibody comprising a VH and VL of any of the PSMA antibodies provided in Tables 4-12 or 23-28. In other embodiments, the antibody is a PSMA antibody comprising a VH CDR1, VH CDR3 and VH CDR3 of any of the PSMA antibodies provided in Tables 4-12 or 23-28. In other embodiments, the antibody is a PSMA antibody comprising a VL CDR1, VL CDR3 and VL CDR3 of any of the PSMA antibodies provided in Tables 4-12 or 23-28. In other embodiments, the antibody is a PSMA antibody comprising a VH CDR1, VH CDR3, VH CDR3, VL CDR1, VL CDR2, and VL CDR3 of any of the PSMA antibodies provided in Tables 4-12 or 23-28. In some embodiments, the VH CDR1, VH CDR2, VH CDR3, VL CDR1, VL CDR2, and VL CDR3 amino acid sequences of the PSMA antibody are according to the Kabat numbering system. In some embodiments, the VH CDR1, VH CDR2, VH CDR3, VL CDR1, VL CDR2, and VL CDR3 amino acid sequences of the PSMA antibody are according to the Chothia numbering system. In some embodiments, the VH CDR1, VH CDR2, VH CDR3, VL CDR1, VL CDR2, and VL CDR3 amino acid sequences of the PSMA antibody are according to the AbM numbering system. In some embodiments, the VH CDR1, VH CDR2, VH CDR3, VL CDR1, VL CDR2, and VL CDR3 amino acid sequences of the PSMA antibody are according to the Contact numbering system. In some embodiments, the VH CDR1, VH CDR2, VH CDR3, VL CDR1, VL CDR2, and VL CDR3 amino acid sequences of the PSMA antibody are according to the IMGT numbering system. In some embodiments, the VH CDR1, VH CDR2, VH CDR3, VL CDR1, VL CDR2, and VL CDR3 amino acid sequences of the PSMA antibody are according to a combination of the numbering systems provided herein.

Also provided herein is a method of treating a disease or disorder in a subject, comprising administering to the subject a PSMA antibody provided herein. Also provided herein is a method of treating a disease or disorder in a subject, comprising administering to the subject a PSMA antigen binding fragment provided herein. Also provided herein is a method of treating a disease or disorder in a subject, comprising administering to the subject a pharmaceutical composition comprising a PSMA antibody provided herein. Also provided herein is a method of treating a disease or disorder in a subject, comprising administering to the subject a pharmaceutical composition comprising a PSMA antigen binding fragment provided herein.

In some embodiments, the disease or disorder is a disease or disorder of the prostate. In some embodiments, the disease or disorder is a prostate-related disease or disorder. In some embodiments, the disease or disorder is prostate cancer. In some embodiments, the prostate cancer is a primary prostate cancer. In some embodiments, the prostate cancer is a metastatic prostate cancer. In another embodiment, the prostate cancer is a castration-resistant cancer. In one embodiment, the disease or disorder of the prostate is a prostate intraepithelial neoplasia. In some embodiments, the disease or disorder of the prostate is a prostate tumor. In some embodiments, the prostate tumor is a solid tumor.

In certain embodiments, the disease or disorder is a colorectal cancer. In certain embodiments, the disease or disorder is a clear cell renal carcinoma. In other embodiments, In certain embodiments, the disease or disorder is a renal cell carcinoma (RCC). In some embodiments, the RCC is a kidney clear cell carcinoma. In some embodiments, the RCC is a kidney papillary cell carcinoma. In certain embodiments, the disease or disorder is a metastatic lesion of a RCC. In certain embodiments, the disease or disorder is a bladder cancer. In certain embodiments, the disease or disorder is a breast cancer. In certain embodiments, the disease or disorder is a kidney cancer. In certain embodiments, the disease or disorder is a neovascular disorder. In certain embodiments, the disease or disorder is a cancer characterized by solid tumor growth. In some embodiments, the neovascular disorder is a clear cell renal carcinoma. In certain embodiments, the disease or disorder is a CCRCC colorectal cancer. In certain embodiments, the disease or disorder is a breast cancer. In certain embodiments, the disease or disorder is a bladder cancer. In certain embodiments, the disease or disorder is a lung cancer. In certain embodiments, the disease or disorder is a pancreatic cancer. In certain embodiments, the disease or disorder is a non-prostate cancers. In certain embodiments, the disease or disorder is a renal cancer. In certain embodiments, the disease or disorder is a urothelial cancer. In certain embodiments, the disease or disorder is a lung cancer. In certain embodiments, the disease or disorder is a colon cancer. In certain embodiments, the disease or disorder is a breast cancer. In certain embodiments, the disease or disorder is a liver adenocarcinaoma.

The cancer can be a hyperproliferative condition or disorder, a solid tumor, a neovasculature, a soft tissue tumor, or a metastatic lesion. “Cancer” is meant to include all types of cancerous growths or oncogenic processes, metastatic tissues or malignantly transformed cells, tissues, or organs, irrespective of histopathology type or stage of invasiveness. Examples of cancers include solid tumors, hematological malignancies, soft tissue tumors, and metastatic lesions. Exemplary solid tumors include malignancies, e.g., sarcomas, and carcinomas (including adenocarcinomas and squamous cell carcinomas) of the various organ systems, such as those affecting prostate, liver, lung, breast, lymphoid, gastrointestinal (e.g., colon), genitourinary tract (e.g., renal, urothelial cells), prostate and pharynx. Adenocarcinomas include malignancies such as most colon cancers, a rectal cancer, a renal-cell carcinoma, a liver cancer, a non-small cell carcinoma of the lung, a cancer of the small intestine and a cancer of the esophagus. Squamous cell carcinomas include malignancies, e.g., in the lung, esophagus, skin, head and neck region, oral cavity, anus, and cervix.

In one specific embodiment, the cancer is a prostate cancer.

Metastatic lesions of the aforementioned cancers can also be treated or prevented using the methods and antibodies described herein.

Exemplary cancers whose growth can be inhibited or reduced using the antibodies described herein include cancers that can overexpress PSMA. Exemplary such cancers include a prostate cancer or a prostatic intraepithelial neoplasia, a colorectal cancer, a gastric cancer, a clear cell renal carcinoma, a bladder cancer, a lung cancer, a squamous cell carcinoma, a glioma, a breast cancer, a kidney cancer, a neovascular disorder, a clear cell renal carcinoma (CCRCC), and a pancreatic cancer and various other non-prostate cancers, including but not limited to a renal cancer, a urothelial cancer and an adenocarcinaoma to the liver. Refractory or recurrent malignancies can be treated using the antibodies described herein.

Exemplary other cancers that can be treated with the antibodies described herein are anal cancer, a basal cell carcinoma, a biliary tract cancer, a bladder cancer, a bone cancer, brain and CNS cancers, a carcinoma of the fallopian tubes, carcinoma of the vagina, a carcinoma of the vulva, a cutaneous or intraocular malignant melanoma, a astro-esophageal cancer, a testicular cancer, an ovarian cancer, a pancreatic cancer, a rectal cancer, an uterine cancer, a primary CNS lymphoma; a neoplasm of the central nervous system (CNS), a cervical cancer, a choriocarcinoma, a rectum cancer, a connective tissue cancer, a cancer of the digestive system, an endometrial cancer, an eye cancer; an intra-epithelial neoplasm, a kidney cancer, a larynx cancer, a liver cancer; a small cell lung cancer, a neuroblastoma, an oral cavity cancer (e.g., lip, tongue, mouth, and pharynx), a nasopharyngeal cancer, a retinoblastoma, a rhabdomyosarcoma, a cancer of the respiratory system, a sarcoma, a thyroid cancer, a cancer of the urinary system, a hepatocarcinoma, a cancer of the anal region, a carcinoma of the fallopian tubes, a carcinoma of the vagina, a carcinoma of the vulva, a cancer of the small intestine, a cancer of the endocrine system, a cancer of the parathyroid gland, a cancer of the adrenal gland, a sarcoma of soft tissue, a cancer of the urethra, a cancer of the penis, solid tumors of childhood, a tumor angiogenesis, a spinal axis tumor, a brain stem glioma, a pituitary adenoma, Kaposi's sarcoma, Merkel cell cancer, an epidermoid cancer, a squamous cell cancer, an environmentally induced cancers including those induced by asbestos, as well as other carcinomas and sarcomas, and combinations of said cancers.

In one embodiment, the cancer is a renal cancer. Renal cancer is one of the 10 most common cancers, affecting about 1 in every 63 people over a lifetime, mostly adults aged between 50 and 80 years. Worldwide, North America has the highest rate of renal cancer, but in developing countries, the incidence has been steadily increasing over the last three decades. Metastatic renal cell carcinoma (mRCC) is a disease that portends poor prognosis despite an increasing number of novel systemic treatment options including new targeted therapies and immunotherapy. PSMA is a transmembrane glycoprotein comprised of 750 amino acids and 3 protein domains; a small intracellular domain, a single-pass transmembrane domain, and a large extracellular domain. PSMA has been reported to be expressed within the neovasculature of other solid tumors including lung, bladder, and renal cancer (Chang S S, et al. Cancer Res. 1999; 59(13):3192-3198). In a recent study examining PSMA expression in renal cell carcinomas (RCC), immunohistochemistry results revealed that endothelial PSMA protein was detected in 80% of clear cell renal carcinomas, 14% of papillary carcinomas, and 72% of chromophobe carcinomas (Spatz S, Tolkach et al. J Urol. 2018; 199(2):370-377). Further analysis from the same study demonstrated that in both clear cell and papillary renal carcinomas, PSMA expression was significantly associated with lower overall survival rates in patients. In another clinical study, a PSMA-based radiotracer using 68Ga was able to detect PSMA in metastatic lesions found in patients with clear cell carcinoma (Sawicki L M, et al. Eur J Nucl Med Mol Imaging. 2017; 44(1):102-107). Thus, in addition to prostate cancer, PSMAxCD3 approaches can also have therapeutic benefit in patients with histologies such as clear cell renal cell carcinoma.

PSMA is highly expressed in prostate cancer and has also been reported to be expressed within the neovasculature of other solid tumors including lung, bladder, and renal cancer. In a recent study examining PSMA expression in renal cell carcinomas (RCC), immunohistochemistry results revealed that endothelial PSMA protein was detected in 80% of clear cell renal carcinomas, 14% of papillary carcinomas, and 72% of chromophobe carcinomas. Further analysis from the same study demonstrated that in both clear cell and papillary renal carcinomas, PSMA expression was significantly associated with lower overall survival rates in patients. In another clinical study, a PSMA-based radiotracer using ⁶⁸Ga was able to detect PSMA in metastatic lesions found in patients with clear cell carcinoma. Thus, PSMA antibodies provided herein can have therapeutic benefit in patients with histologies, such as clear cell renal cell carcinoma.

In one specific embodiment, the cancer is a renal cancer. In another specific embodiment, the cancer is a renal cell carcinoma. In yet another specific embodiment, the cancer is a metastatic renal cell carcinoma. In other specific embodiments, the cancer is a clear cell renal cell carcinoma. In other specific embodiments, the cancer comprises PSMA-expressing cells.

Patients having cancer including metastatic cancer that express PSMA can be treated with the antibodies described herein. The cancer can be a prostate cancer or a prostatic intraepithelial neoplasia, a colorectal cancer, a gastric cancer, a clear cell renal carcinoma, a bladder cancer, a lung cancer, a squamous cell carcinoma, a glioma, a breast cancer, a kidney cancer, a neovascular disorder, a clear cell renal carcinoma (CCRCC), and a pancreatic cancer and various other non-prostate cancers, including but not limited to a renal cancer, a urothelial cancer and an adenocarcinaoma to the liver.

In some embodiments, the subject has a solid tumor. In some embodiments, the subject has a prostate tumor. In some embodiments, the solid tumor is a colorectal cancer. In some embodiments, the solid tumor is a gastric cancer. In some embodiments, the solid tumor is a lung cancer. In some embodiments, the solid tumor is a bladder cancer. In some embodiments, the solid tumor is a squamous cell carcinoma. In some embodiments, the cancer is CCRCC. In some embodiments, the solid tumor is a breast cancer. In some embodiments, the solid tumor is glioma. In some embodiments, the solid tumor is a prostate cancer or castration-resistant prostate cancer. In some embodiments, the solid tumor is a kidney cancer. In some embodiments, the solid tumor is a pancreatic cancer. In some embodiments, the solid tumor is an adenocarcinoma to the liver. In some embodiments, the cancer is a neovascular. In some embodiments, the cancer is renal cancer. In some embodiments, the solid tumor is a urothelial cancer. In some embodiments, the solid tumor is a brain cancer.

In some embodiments, the subject has a tumor that expresses PSMA. In some embodiments, the subject has tumor-infiltrating T lymphocytes (TILs) in the tumor tissue.

“Increased number” refers to statistically significant increase in a subject when compared to a control. “Increased number” for example refers to statistically significant increase in the number of TILs in a subject (e.g. patient) pre- and post-treatment with a PSMA antibody or other therapeutic.

An exemplary PSMA positive cancer is a prostate cancer. Another exemplary PSMA positive cancer is a renal cancer. In some embodiments, prostate cancer is adenocarcinoma. In some embodiments, prostate cancer is a metastatic prostate cancer. In some embodiments, prostate cancer has metastasized to rectum, lymph node or bone, or any combination thereof. In some embodiments, prostate cancer is relapsed or refractory prostate cancer. In some embodiments, prostate cancer is castration resistant prostate cancer. In some embodiments, prostate cancer is sensitive to androgen deprivation therapy. In some embodiments, prostate cancer is insensitive to androgen deprivation therapy.

In some embodiments described herein, the subject has increased expression or activity of interferon-gamma (IFN-γ).

In some embodiments described herein the subject has been treated with an anti-PSMA antibody. In some embodiments described herein, the subject is refractory to treatment with the anti-PSMA antibody.

Any of the PSMA or bispecific PSMAxCD3 antibodies described herein can be used in the methods provided herein.

Also provided is a method of directing CD3-expressing T cells to a second target. The methods can comprise contacting the CD3-expressing T cell with a multispecific PSMAxCD3 antibody or antigen binding fragment thereof provided herein, wherein the multispecific PSMAxCD3 antibody or antigen binding fragment thereof directs the CD3-expressing T cell to the second target. Also provided is a method of directing a T cell expressing CD3 to a second target, the method comprising contacting the T cell with a multispecific PSMAxCD3 antibody provided herein, wherein the contacting directs the T cell to the second target. In certain embodiments, the antibody is a multispecific PSMAxCD3 antibody provided herein. In some embodiments, the second target is PSMA. In some embodiments, the second target is a cell expressing PSMA. In some embodiments, the second target is a prostate cell. In some embodiments, the second target is a prostate cell expressing PSMA. In some embodiments, the second target is a prostate cancer cell. In some embodiments, the second target is a prostate cancer cell expressing PSMA. In some embodiments, the second target is a renal cell. In some embodiments, the second target is a renal cancer cell. In certain embodiments, the second target is a renal cell expressing PSMA.

Also provided is a method for inhibiting growth or proliferation of target cells. The methods can comprise contacting the CD3-expressing T cells with a multispecific PSMAxCD3 antibody or antigen binding fragment thereof provided herein, wherein contacting the target cells with the multispecific PSMAxCD3 antibody or antigen binding fragment thereof composition inhibits the growth or proliferation of the target cells. In some embodiments, the target cell is a cell expressing PSMA. In some embodiments, the target cell is a prostate cell. In some embodiments, the target cell is a prostate cell expressing PSMA. In some embodiments, the target cell is a prostate cancer cell. In some embodiments, the target cell is a prostate cancer cell expressing PSMA. In some embodiments, the target cell is a renal cell. In some embodiments, the target cell is a renal cancer cell. In certain embodiments, the target cell is a renal cell expressing PSMA.

Also provided is a method of inhibiting growth or proliferation of target cells expressing a second target antigen on the cell surface, the method comprising contacting the target cells with a multispecific PSMA antibody provided herein, wherein contacting the target cells with the pharmaceutical composition inhibits growth or proliferation of the target cells. In some embodiments, the target cells are in the presence of a T cell expressing CD3 while in contact with the multispecific antibody. In certain embodiments, the antibody is a multispecific PSMAxCD3 antibody provided herein. In some embodiments, the target cell is a cell expressing PSMA. In some embodiments, the target cell is a prostate cell. In some embodiments, the target cell is a prostate cell expressing PSMA. In some embodiments, the target cell is a prostate cancer cell. In some embodiments, the target cell is a prostate cancer cell expressing PSMA. In some embodiments, the target cell is a renal cell. In some embodiments, the target cell is a renal cancer cell. In certain embodiments, the target cell is a renal cell expressing PSMA.

Also provided herein is a method of treating a disease or disorder in a subject, comprising administering to the subject a PSMA multispecific antibody provided herein. Also provided herein is a method of treating a disease or disorder in a subject, comprising administering to the subject a multispecific antibody comprising a PSMA antigen binding fragment provided herein. Also provided herein is a method of treating a disease or disorder in a subject, comprising administering to the subject a pharmaceutical composition comprising a PSMA multispecific antibody provided herein. Also provided herein is a method of treating a disease or disorder in a subject, comprising administering to the subject a pharmaceutical composition comprising a multispecific antibody comprising a PSMA antigen binding fragment provided herein. In certain embodiments, the antibody is a multispecific PSMAxCD3 antibody provided herein. In some embodiments, the disease or disorder is a disease or disorder of the prostate. In some embodiments, the disease or disorder is a prostate-related disease or disorder. In some embodiments, the disease or disorder is prostate cancer. In some embodiments, the disease or disorder is a renal disease or disorder. In some embodiments, the disease or disorder is a renal cancer. In some embodiments, the disease or disorder is a renal cell carcinoma. In some embodiments, the renal cell carcinoma is a metastatic renal cell carcinoma.

In certain embodiments of the methods provided herein, the disease or disorder is mediated by a PSMA-expressing cell.

In another general aspect, provided herein is a method of treating a disease or disorder in a subject, comprising administering to the subject an isolated multispecific antibody or antigen binding fragment thereof that specifically binds PSMA and a second target antigen presented on the surface of a T cell, or a pharmaceutical composition disclosed herein. In some embodiments, the second target antigen is CD3. In certain embodiments, the antibody is a multispecific PSMAxCD3 antibody provided herein. In some embodiments, the disease or disorder is a disease or disorder of the prostate. In some embodiments, the disease or disorder is a prostate-related disease or disorder. In some embodiments, the disease or disorder is prostate cancer. In some embodiments, provided is a method for eliminating target cells expressing PSMA or treating a disease caused all or in part by target cells expressing the PSMA in a subject, comprising administering an effective amount of a multispecific PSMA antibody provided herein to the subject. In certain embodiments, the antibody is a multispecific PSMAxCD3 antibody.

In some embodiments, the subject is a subject in need thereof. In some embodiments, the subject is a human. In specific embodiments, the subject is administered an effective amount. In some embodiments, the subject has a prostate cancer. In some embodiments, the subject has a primary prostate cancer. In one embodiment, the subject has a metastatic prostate cancer. In certain embodiments, the subject is hormone-refractory. PSMA can be used as a biomarker for disease, disease progression, or disease reoccurrence in a subject. In some embodiments, the subject has detectable levels of PSMA in the blood. In some embodiments, the subject has detectable levels of PSMA in the bone marrow. Methods of detection of PSMA are known in the art. In some embodiments, the PSMA is detected using a PSMA antibody provided herein. In some embodiments, the subject has detectable levels of prostate-specific antigen (PSA) in the blood. In other embodiments, the subject has detectable levels of PSA in the bone marrow. Methods of detection of PSA are known in the art.

As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a disease or disorder (e.g., a cancer), which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In some embodiments, a PSMA antibody provided herein is used in combination with a supplemental therapy. In some embodiments, a multispecific PSMAxCD3 antibody provided herein is used in combination with a supplemental therapy. In some embodiments, the supplemental therapy is surgery. In some embodiments, the supplemental therapy is radiation. In some embodiments, the supplemental therapy is hormone therapy. In certain embodiments, the hormone therapy is abiraterone. In certain embodiments, the hormone therapy is enzalutamide. In some embodiments, the supplemental therapy is chemotherapy. In certain embodiments, the chemotherapy is docetaxel. In certain embodiments, the chemotherapy is cabazitaxel. In some embodiments, the supplemental therapy is a cancer vaccine. In some embodiments, the supplemental therapy is a radiopharmaceutical agent (e.g., radium-223 chloride).

As used herein, the term “in combination,” in the context of the administration of two or more therapies to a subject, refers to the use of more than one therapy. The use of the term “in combination” does not restrict the order in which therapies are administered to a subject. “In combination with” can include two or more therapeutics are administered to a subject together in a mixture, concurrently as single agents or sequentially as single agents in any order. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

The antibodies described herein can also be administered in combination therapy, i.e., combined with other therapeutic agents relevant for the disease or condition to be treated. Accordingly, in one embodiment, the antibody-containing medicament is for combination with one or more further therapeutic agent, such as a chemotherapeutic agent. In some embodiments, the other therapeutic agent is a radiopharmaceutical agent (such as radium-223 chloride), secondary hormone therapies (such as abiraterone or enzalutamide), and/or chemotherapies (docetaxel and cabazitaxel). Such combined administration can be simultaneous, separate or sequential, in any order. For simultaneous administration the agents can be administered as one composition or as separate compositions, as appropriate. a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin, ARN-509), serine or tyrosine kinase inhibitor (e.g., PI3 kinase inhibitor SF1126) (e.g., lapatanib), multikinase inhibitor (e.g., sorafenib, sunitinib), VEGF inhibitor (e.g., bevacizumab), TAK-700, cancer vaccine (e.g., BPX-101, PEP223), Listeria-based vaccine, lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

In one embodiment, a method for treating a disorder involving cells expressing PSMA in a subject, which method comprises administration of a therapeutically effective amount of a bispecific antibody or fragment, such as a PSMA×CD3 bispecific antibody described herein, and radiotherapy to a subject in need thereof is provided. In one embodiment is provided a method for treating or preventing cancer, which method comprises administration of a therapeutically effective amount of a bispecific antibody or fragment, such as a PSMA×CD3 antibody described herein, and radiotherapy to a subject in need thereof. Radiotherapy can comprise radiation or associated administration of radiopharmaceuticals to a patient is provided. The source of radiation can be either external or internal to the patient being treated (radiation treatment can, for example, be in the form of external beam radiation therapy (EBRT) or brachytherapy (BT)). Radioactive elements that can be used in practicing such methods include, e.g., radium, cesium-137, iridium-192, americium-241, gold-198, cobalt-57, copper-67, technetium-99, iodide-123, iodide-131, and indium-111.

Antibodies described herein can be administered in combination with a vaccine. Exemplary vaccines are immunogenic agents, such as cancerous cells, purified tumor antigens (including recombinant proteins, antigen epitopes, peptides and carbohydrate molecules), tumor antigens delivered to a patient via gene therapy, cells, and cells transfected with genes encoding immune stimulating cytokines. Exemplary vaccines that can be used include peptides of melanoma antigens, such as peptides of gp100, MAGE antigens, Trp-2, MARTI_ and/or tyrosinase, or tumor cells transfected to express the cytokine GM-CSF, DNA-based vaccines, RNA-based vaccines, Listeria-based vaccines and viral transduction-based vaccines, peptides or prostate antigens (e.g., PSMA, STEAP1, PSCA), the cancer vaccine sipuleucel-T or peptides of lung cancer antigens. The cancer vaccine can be prophylactic or therapeutic.

Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al., (1993) Proc Natl Acad Sci U.S.A. 90: 3539-43).

The antibodies described herein can be administered in combination with one or a collection of recombinant proteins and/or peptides expressed in or on a tumor in order to generate an immune response to these proteins. These proteins are normally viewed by the immune system as self-antigens and are therefore tolerant to them. The tumor antigen can also include the protein telomerase, which is required for the synthesis of telomeres of chromosomes and which is expressed in more than 85% of human cancers and in only a limited number of somatic tissues (Kim et al., (1994) Science 266: 2011-2013). Tumor antigens can also be “neo-antigens” expressed in or on cancer cells as a result of somatic mutations that alter protein sequence or create fusion proteins between two unrelated sequences (e.g., bcr-abl in the Philadelphia chromosome), or idiotype from B cell tumors. The tumor antigens can be antigen epitopes of prostate specific antigen (PSA), mesothelin, prostate-specific membrane antigen (PSMA), synovial sarcoma X2 (SSX2), NKX3.1, prostatic acidic phosphatase (PAP), or epidermal growth factor receptors, or peptides specific for variants of EGFR such as the well-known EGFRvIII overexpressed on tumor cells.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV), and Epstein-Barr virus (EBV). Another form of tumor specific antigens which can be used in combination with the antibodies described herein is purified heat shock proteins (HSP) isolated from the tumor tissue itself. HSP contain fragments of proteins from the tumor cells and are highly efficient at delivery to antigen presenting cells for eliciting tumor immunity (Suot and Srivastava (1995) Science 269:1585-1588; Tamura et al., (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al., (1998) Nature Medicine 4: 328-332). DCs can also be transduced by genetic means to express these tumor antigens. DCs have also been fused directly to tumor cells for the purposes of immunization (Kugler et al., (2000) Nature Medicine 6:332-336). As a method of vaccination, DC immunization can be effectively combined with the antibodies described herein to activate more potent anti-tumor responses.

In some embodiments described herein, the antibodies specifically binding PSMA provided herein or the bispecific PSMAxCD3 antibodies provided herein are administered in combination with a tumor vaccine comprising a peptide fragment of a prostate specific antigen, or a vector encoding the peptide fragment of a prostate specific antigen.

The antibodies described herein can be administered in combination with a standard of care cancer treatment.

The antibodies described herein can be administered in combination with a standard of care cancer chemotherapeutic regimes. In these instances, it can be possible to reduce the dose of chemotherapeutic reagent administered (Mokyr et al., (1998) Cancer Research 58: 5301-5304).

In some embodiments described herein, the antibodies provided herein can be administered in combination with one or more of other antibody molecules, chemotherapy, other anti-cancer therapy (e.g., targeted anti-cancer therapies, or oncolytic drugs), cytotoxic agents, cytokines, surgical and/or radiation procedures.

Exemplary cytotoxic agents that can be administered in combination with the antibodies described herein include hormone inhibitors, antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation).

Standard of care therapeutics include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), Ibrutinib, idelalisib, and brentuximab vedotin.

Exemplary alkylating agents include, nitrogen mustards, ethylenimine derivatives, alkyl sulfonates, nitrosoureas and triazenes: uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines include, e.g., doxorubicin (Adriamycin® and Rubex®); bleomycin (Lenoxane®); daunorubicin (daunorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids that can be used in combination with the antibodies provided herein include vinorelbine tartrate (Navelbine®), Vincristine (Oncovin®), and Vindesine (Eldisine®); vinblastine (also known as vinblastine sulfate, vincaleukoblastine and VLB, Alkaban-AQ® and Velban®); and vinorelbine (Navelbine®).

Exemplary proteosome inhibitors that can be used in combination with the antibodies provided herein, alone or in combination with another immunomodulator are bortezomib (Velcade®); carfilzomib (PX-171-007, (S)-4-Methyl-N—((S)-1-4(S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In some embodiments described herein, the antibodies provided herein are administered in combination with a serine or tyrosine kinase inhibitor (e.g., a receptor tyrosine kinase (RTK) inhibitor). Exemplary tyrosine kinase inhibitor include an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor), a platelet derived growth factor (PDGF) pathway inhibitor (e.g., a platelet derived growth factor receptor (PDGFR) inhibitor (e.g., a PDGFR-β inhibitor), a RAF-1 inhibitor, a KIT inhibitor and a RET inhibitor. In some embodiments, the second therapeutic is axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In some embodiments, the EGFR inhibitor is a bispecific EGFRc-Met antibody (EM-1 mAb) comprising the heavy and the light chains of SEQ ID NOs 249, 250, 217 and 218 (US2014/0141000).

In some embodiments, the antibodies provided herein are administered in combination with Vascular Endothelial Growth Factor (VEGF) receptor inhibitors, including Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxy phenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary VEGF inhibitors include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al., (1997) Cancer Res 57:4593-4599. In one embodiment, the anti-VEGF antibody is Bevacizumab (BV), also known as rhuMAb VEGF or AVASTIN®. It comprises mutated human IgG1 framework regions and antigen-binding complementarity-determining regions from the murine anti-hVEGF monoclonal antibody A.4.6.1 that blocks binding of human VEGF to its receptors. Bevacizumab and other humanized anti-VEGF antibodies are further described in U.S. Pat. No. 6,884,879. Additional anti-VEGF antibodies include the G6 or B20 series antibodies (e.g., G6-31, B20-4.1), as described in Int. Patent Publication Nos. WO2005/012359 and WO2005/044853, For additional antibodies see U.S. Pat. Nos. 7,060,269, 6,582,959, 6,703,020, 6,054,297, WO98/45332, WO 96/30046, WO94/10202, EP 0666868B1, U.S. Patent Application Publication Nos. US2006009360, US20050186208, US20030206899, US20030190317, US20030203409, and US20050112126; and Popkov et al., (2004) Journal of Immunological Methods 288: 149-164. Other antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183 and Q89.

In some embodiments described herein, the antibodies provided herein are administered in combination with a PI3K inhibitor. In one embodiment, the PI3K inhibitor is an inhibitor of delta and gamma isoforms of PI3K. In another embodiment, the PI3K inhibitor is an inhibitor of beat isoforms of PI3K. Exemplary PI3K inhibitors that can be used are described in, e.g., WO 2010/036380, WO 2010/006086, WO 09/114870, WO 05/113556, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235).

In some embodiments described herein, the antibodies provided herein are administered in combination with a mTOR inhibitor, e.g., one or more mTOR inhibitors chosen from one or more of rapamycin, temsirolimus (TORISEL®), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587. ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-12,4-Bis[(3 S)-3-methylmorpholin-4-yl] pyrido[2,3-d]pyrimidin-7-yl1-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO:237), inner salt (SF1126, CAS 936487-67-1), and XL765.

In some embodiments described herein, the antibodies provided herein are administered in combination with a BRAF inhibitor, e.g., GSK2118436, RG7204, PLX4032, GDC-0879, PLX4720, and sorafenib tosylate (Bay 43-9006).

In some embodiments described herein, the antibodies provided herein are administered in combination with an immunomodulatory agent. Targeting immune checkpoints such as programmed cell death protein 1 (PD1), programmed cell death 1 ligand 1 (PDL1) and cytotoxic T lymphocyte antigen 4 (CTLA4) has achieved noteworthy benefit in multiple cancers by blocking immunoinhibitory signals and enabling patients to produce an effective antitumour response. In some embodiments, the antibodies provided herein are administered in combination with an anti-PD1 (eg. nivolumab), anti-PDL (eg. MDX-1105) or anti-CTLA4 (eg. Ipilimumab). The ability of agonistic CD40 antibodies (referred to as αCD40) or CD40 ligand to stimulate immune responses and target tumors suggests such reagents have promise as cancer immunotherapeutics. In some embodiments, the antibodies provided herein are administered in combination with an anti-CD40 (eg. SGN-40, CP-870,893) or anti-CD40L (eg. BG9588).

In some embodiments described herein, the antibodies provided herein are administered in combination with a MEK inhibitor.

In some embodiments described herein, the antibodies provided herein administered in combination with the MEK inhibitor are used in the treatment of a prostate cancer, a melanoma, a colorectal cancer, a non-small cell lung cancer, an ovarian cancer, a breast cancer, a prostate cancer, a pancreatic cancer, a hematological malignancy or a renal cell carcinoma. In certain embodiments, the tumor tissue or cancer cell has a BRAF mutation (e.g., a BRAF V600E mutation), a BRAF wildtype, a KRAS wildtype or an activating KRAS mutation. The cancer can be at an early, intermediate or late stage. Any MEK inhibitor can be used in combination including, ARRY-142886, G02442104 (also known as GSK1120212), RDEA436, RDEA119/BAY 869766, AS703026, G00039805 (also known as AZD-6244 or selumetinib), BIX 02188, BIX 02189, CI-1040 (PD-184352), PD0325901, PD98059, U0126, GDC-0973 (Methanone, [3,4-difluoro-2-[(2-fluoro-4-iodophenyl)amino]phenyl][3-hydroxy-3-(25)-2-piperidinyl-1-azetidinyl]-), G-38963, G02443714 (also known as AS703206), or a pharmaceutically acceptable salt or solvate thereof. Additional examples of MEK inhibitors are disclosed in WO 2013/019906, WO 03/077914, WO 2005/121142, WO 2007/04415, WO 2008/024725 and WO 2009/085983.

In some embodiments described herein, the antibodies provided herein are administered in combination with a JAK2 inhibitor, e.g., CEP-701, INCB18424, CP-690550 (tasocitinib).

In some embodiments described herein, the antibodies provided herein are administered in combination with paclitaxel or a paclitaxel agent, e.g., TAXOL®, protein-bound paclitaxel (e.g., ABRAXANE®). Exemplary paclitaxel agents include nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., (2007) Bioorganic & Medicinal Chemistry Letters 17:617-620).

In some embodiments described herein, the antibodies provided herein are administered in combination with a cellular immunotherapy (e.g., Provenge (e.g., Sipuleucel)), and optionally in combination with cyclophosphamide.

Exemplary therapeutic agents that can be used in combination with the antibodies provided herein for treatment of a cancer include a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, ARN-509, radiation therapy, surgery, or a combination thereof.

Exemplary therapeutic agents that can be used in combination with the antibodies provided herein for treatment of a cancer include a chemotherapeutic agent, e.g., paclitaxel or a paclitaxel agent (e.g., a paclitaxel formulation such as TAXOL, an albumin-stabilized nanoparticle paclitaxel formulation (e.g., ABRAXANE) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus); IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); Raf/MEK dual kinase inhibitor (e.g., RO5126766); Notch signaling inhibitor (e.g., MK0752); monoclonal antibody-antibody fusion protein (e.g., L19IL2); curcumin; HSP90 inhibitor (e.g., tanespimycin, STA-9090); rIL-2; denileukin diftitox; topoisomerase 1 inhibitor (e.g., irinotecan, PEP02); statin (e.g., simvastatin); Factor VIIa inhibitor (e.g., PCI-27483); AKT inhibitor (e.g., RX-0201); hypoxia-activated prodrug (e.g., TH-302); metformin hydrochloride, gamma-secretase inhibitor (e.g., R04929097); ribonucleotide reductase inhibitor (e.g., 3-AP); immunotoxin (e.g., HuC242-DM4); PARP inhibitor (e.g., KU-0059436, veliparib); CTLA-4 inhbitor (e.g., CP-675,206, ipilimumab); AdV-tk therapy; proteasome inhibitor (e.g., bortezomib (Velcade), NPI-0052); thiazolidinedione (e.g., pioglitazone); NPC-1C; Aurora kinase inhibitor (e.g., R763/AS703569), CTGF inhibitor (e.g., FG-3019); siG12D LODER; and radiation therapy (e.g., tomotherapy, stereotactic radiation, proton therapy), surgery, and a combination thereof. In certain embodiments, a combination of paclitaxel or a paclitaxel agent, and gemcitabine can be used with the antibodies provided herein.

Exemplary therapeutic agents that can be used in combination with the antibodies provided herein for treatment of small cell lung cancer include a chemotherapeutic agent, e.g., etoposide, carboplatin, cisplatin, oxaliplatin, irinotecan, topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., tanespimycin, STA-9090); mTOR inhibitor (e.g., everolimus); Ep-CAM/CD3-bispecific antibody (e.g., MT110); CK-2 inhibitor (e.g., CX-4945); HDAC inhibitor (e.g., belinostat); SMO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof.

Exemplary therapeutic agents that can be used in combination with the antibodies provided herein for treatment of non-small cell lung cancer include a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (e.g., EGFR inhibitor (e.g., erlotinib, gefitinib, cetuximab, panitumumab, necitumumab, PF-00299804, nimotuzumab, R05083945), MET inhibitor (e.g., PF-02341066, ARQ 197), PI3K kinase inhibitor (e.g., XL147, GDC-0941), Raf/MEK dual kinase inhibitor (e.g., R05126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine, GVAX, recombinant DNA and adenovirus expressing L523S protein), Bcl-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., tanespimycin, STA-9090, AUY922, XL888), mTOR inhibitor (e.g., everolimus, temsirolimus, ridaforolimus), Ep-CAM/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide (SEQ ID NO:225)-TNF alpha conjugate, dichloroacetate (DCA), HGF inhibitor (e.g., SCH 900105), SAR240550, PPAR-gamma agonist (e.g., CS-7017), gamma-secretase inhibitor (e.g., R04929097), epigenetic therapy (e.g., 5-azacitidine), nitroglycerin, MEK inhibitor (e.g., AZD6244), cyclin-dependent kinase inhibitor (e.g., UCN-01), cholesterol-Fus1, antitubulin agent (e.g., E7389), farnesyl-OH-transferase inhibitor (e.g., lonafarnib), immunotoxin (e.g., BB-10901, SSI (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), EGFR/c-Met bispecific antibody EM-1 as described in US2014/0141000A1, radiation therapy, surgery, and combinations thereof.

Exemplary therapeutic agents that can be used in combination with the antibodies provided herein for treatment of ovarian cancer include a chemotherapeutic agent (e.g., paclitaxel or a paclitaxel agent; docetaxel; carboplatin; gemcitabine; doxorubicin; topotecan; cisplatin; irinotecan, TLK286, ifosfamide, olaparib, oxaliplatin, melphalan, pemetrexed disodium, SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agent (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., R04929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy, and combinations thereof.

Exemplary therapeutics agents that can be used in combination with the antibodies provided herein for treatment of a renal cancer, e.g., a renal cell carcinoma (RCC) or metastatic RCC include an immune-based strategy (e.g., interleukin-2 or interferon-α), a targeted agent (e.g., a VEGF inhibitor such as a monoclonal antibody to VEGF, e.g., bevacizumab (Rini et al., (2010) J Clin Oncol 28(13):2137-2143)); a VEGF tyrosine kinase inhibitor such as sunitinib, sorafenib, axitinib and pazopanib (reviewed in Pal et al., (2014) Clin Advances in Hematology & Oncology 12(2):90-99); an RNAi inhibitor, or an inhibitor of a downstream mediator of VEGF signaling, e.g., an inhibitor of the mammalian target of rapamycin (mTOR), e.g., everolimus and temsirolimus (Hudes et al., (2007) N Engl J Med 356(22):2271-2281, Motzer et al., (2008) Lancet 372: 449-456).

An “immunoconjugate” refers to the antibody provided herein conjugated to one or more heterologous molecule(s). In some embodiments, a PSMA antibody provided herein is an immunoconjugate. In some embodiments, a multispecific PSMA antibody provided herein is an immunoconjugate. In certain embodiments, the multispecific PSMA antibody is a multispecific PSMAxCD3 antibody provided herein.

In some embodiments, the antibody is conjugated to one or more cytotoxic agents. Exemplary such cytotoxic agents include chemotherapeutic agents or drugs, growth inhibitory agents, toxins (e.g., protein toxins, enzymatically active toxins of bacterial, fungal, plant, or animal origin, or fragments thereof), and radioactive isotopes.

In some embodiments, an immunoconjugate is an antibody-drug conjugate (ADC) in which the antibody provided herein is conjugated to one or more drugs. In other embodiments, a PSMA antibody provided herein is an ADC. In other embodiments, a multispecific PSMA antibody provided herein is an ADC. In certain embodiments, the multispecific PSMA antibody is a multispecific PSMAxCD3 antibody provided herein. Exemplary drugs include a maytansinoid (see, e.g., U.S. Pat. Nos. 5,208,020, 5,416,06)); an auristatin such as monomethylauristatin drug moieties DE and DF (MMAE and MMAF) (see, e.g., U.S. Pat. Nos. 5,635,483 and 5,780,588, and 7,498,298), a dolastatin, a calicheamicin or derivative thereof (see, e.g., U.S. Pat. Nos. 5,712,374, 5,714,586, 5,739, 116, 5,767,285, 5,770,701, 5,770,710, 5,773,001, and 5,877,296; Hinman et al., (1993) Cancer Res 53:3336-3342; and Lode et al., (1998) Cancer Res 58:2925-2928); an anthracycline such as daunomycin or doxorubicin (see, e.g., Kratz et al., (2006) Current Med. Chem 13:477-523; Jeffrey et al., (2006) Bioorganic & Med Chem Letters 16:358-362; Torgov et al., (2005) Bioconj Chem 16:717-721; Nagy et al., (2000) Proc Natl Acad Sci USA 97:829-834; Dubowchik et al, Bioorg. & Med. Chem. Letters 12: 1529-1532 (2002); King et al., (2002) J Med Chem 45:4336-4343; and U.S. Pat. No. 6,630,579), methotrexate, vindesine, a taxane such as docetaxel, paclitaxel, larotaxel, tesetaxel, and ortataxel.

In some embodiments, the immunoconjugate comprises the antibody described herein conjugated to an enzymatically active toxin or fragment thereof, such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments described herein, the antibody is conjugated to a radioactive atom to form a radioconjugate. A variety of radioactive isotopes are available for the production of radioconjugates. Examples include At211, 1131, 1125, Y90, Re186, Re188, Sm153, Bi212, P32, Pb212 and radioactive isotopes of Lu. When the radioconjugate is used for detection, it can comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-I 1, fluorine-19, carbon-13, nitrogen-15, oxygen-17, gadolinium, manganese or iron.

Conjugates of the antibodies described herein and the cytotoxic agent can be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HQ), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., (1987) Science 238: 1098. Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., (1992) Cancer Res 52: 127-131; U.S. Pat. No. 5,208,020) can be used.

The immunoconjugates or ADCs can be prepared with cross-linker reagents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In one embodiment, provided is an immunoconjugate comprising a PSMA antibody provided herein linked to a therapeutic agent or an imaging agent. In another embodiment, provided is an is an immunoconjugate comprising a CD3 antibody provided herein to a therapeutic agent or an imaging agent. In one embodiment, provided is an immunoconjugate comprising a bispecific PSMA/CD3 antibody provided herein linked to a therapeutic agent or an imaging agent.

PSMA antibodies provided herein can also be used as agents to detect PSMA-expressing cells. Thus, in another methods, provided is a method of detecting a cell expressing PSMA, comprising contacting a cell with a PSMA antibody provided herein. In some embodiments, the antibody is a multispecific PSMA antibody. In some embodiments, the PSMA antibody is a multispecific PSMAxCD3 antibody. In certain embodiments, the detecting is by ELISA. In some embodiments, the detecting is by FACS analysis. Also provided are kits comprising a PSMA antibody provided herein, and instructions for use.

### Enrichment and Detection Methods

In one aspect, the PSMA antibodies provided herein are used as agents to detect PSMA-expressing cells. Thus, in other methods, provided is a method of detecting a cell expressing PSMA, comprising contacting a cell with a PSMA antibody provided herein. In certain embodiments, the detecting is by ELISA. In some embodiments, the detecting is by FACS analysis.

In some embodiments, PSMA antibodies provided herein are used in a method to diagnose PSMA-expressing cancer progression. In some embodiments, PSMA antibodies provided herein are used in a method to diagnose PSMA-expressing cancer progression. In some embodiments, PSMA antibodies provided herein are used in a method to diagnose PSMA-expressing cancer regression. In some embodiments, PSMA antibodies provided herein are used in a method to diagnose PSMA-expressing cancer stability.

In some embodiments, PSMA antibodies provided herein are used in a method to monitor PSMA-expressing cancer progression. In some embodiments, PSMA antibodies provided herein are used in a method to monitor PSMA-expressing cancer progression. In some embodiments, PSMA antibodies provided herein are used in a method to monitor PSMA-expressing cancer regression. In some embodiments, PSMA antibodies provided herein are used in a method to monitor PSMA-expressing cancer stability.

In other embodiments, PSMA antibodies provided herein are used in a method to determine whether a patient should be treated for cancer. In some embodiments, PSMA antibodies provided herein are used in a method to determine whether a subject is afflicted with PSMA-expressing cancer. In some embodiments, PSMA antibodies provided herein are used in a method to determine whether a subject is amenable to treatment with a PSMA-specific anti-cancer therapeutic. In certain embodiments, the PSMA-specific anti-cancer therapeutic is a PSMA antibody provided herein. In other embodiments, the PSMA-specific anti-cancer therapeutic multispecific PSMA antibody provided herein. In specific embodiments, the PSMA-specific anti-cancer therapeutic is a multispecific PSMAxCD3 antibody provided herein.

In one aspect, provided is a method of detecting PSMA in a sample, comprising obtaining the sample, contacting the sample with a PSMA antibody provided herein, and detecting the antibody bound to PSMA in the sample.

In another aspect, provided is a method of detecting PSMA and CD3 in a sample, comprising obtaining the sample, contacting the sample with the bispecific antibody comprising a first domain specifically binding PSMA and a second domain specifically binding CD3 provided herein, and detecting the antibody bound to PSMA and CD3 in the sample.

In some embodiments, the sample can be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like.

The antibodies described herein bound to PSMA or PSMA and CD3 can be detected using known methods. Exemplary methods include direct labeling of the antibodies using fluorescent or chemiluminescent labels, or radiolabels, or attaching to the antibodies provided herein a moiety, which is readily detectable, such as biotin, enzymes or epitope tags. Exemplary labels and moieties are ruthenium, 1111n-DOTA, 111In-diethylenetriaminepentaacetic acid (DTPA), horseradish peroxidase, alkaline phosphatase and beta-galactosidase, poly-histidine (HIS tag), acridine dyes, cyanine dyes, fluorine dyes, oxazin dyes, phenanthridine dyes, rhodamine dyes and Alexafluor® dyes.

The antibodies provided herein can be used in a variety of assays to detect PSMA or PSMA and CD3 in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

The PSMA antibodies or multispecific PSMA antibodies provided herein can be used to selectively enrich, isolate, separate, purify, sort, select, capture or detect PSMA-expressing cells. The PSMA antibodies or multispecific PSMA antibodies provided herein can be utilized in a bispecific format, e.g. containing a first antigen binding domain that specifically binds PSMA and a second antigen binding domain that specifically binds a second target. In some embodiments, the second target is CD3. In other embodiments, the multispecific PSMA antibodies provided herein can be utilized in a format that further incorporates a third antigen binding domain that specifically binds a third antigen (e.g., at a trispecific antibody). In other embodiments, the multispecific PSMA antibodies provided herein can be utilized in a format that further incorporates a fourth antigen binding domain that specifically binds a fourth antigen. (e.g., as a quadraspecific antibody).

In one aspect, provided herein is a method of enriching a PSMA-expressing cell comprising: providing a sample comprising the PSMA-expressing cell; contacting the sample with a PSMA antibody provided herein; and enriching the PSMA-expressing cell bound to the PSMA antibody. In one aspect, provided herein is a method of isolating a PSMA-expressing cell comprising: providing a sample comprising the PSMA-expressing cell; contacting the sample with a PSMA antibody provided herein; and isolating the PSMA-expressing cell bound to the PSMA antibody. In one aspect, provided herein is a method of separating a PSMA-expressing cell comprising: providing a sample comprising the PSMA-expressing cell; contacting the sample with a PSMA antibody provided herein; and separating the PSMA-expressing cell bound to the PSMA antibody. In one aspect, provided herein is a method of purifying a PSMA-expressing cell comprising: providing a sample comprising the PSMA-expressing cell; contacting the sample with a PSMA antibody provided herein; and purifying the PSMA-expressing cell bound to the PSMA antibody. In one aspect, provided herein is a method of sorting a PSMA-expressing cell comprising: providing a sample comprising the PSMA-expressing cell; contacting the sample with a PSMA antibody provided herein; and sorting the PSMA-expressing cell bound to the PSMA antibody. In one aspect, provided herein is a method of selecting a PSMA-expressing cell comprising: providing a sample comprising the PSMA-expressing cell; contacting the sample with a PSMA antibody provided herein; and selecting the PSMA-expressing cell bound to the PSMA antibody. In one aspect, provided herein is a method of capturing a PSMA-expressing cell comprising: providing a sample comprising the PSMA-expressing cell; contacting the sample with a PSMA antibody provided herein; and capturing the PSMA-expressing cell bound to the PSMA antibody. In one aspect, provided herein is a method of detecting a PSMA-expressing cell comprising: providing a sample comprising the PSMA-expressing cell; contacting the sample with a PSMA antibody provided herein; and detecting the PSMA-expressing cell bound to the PSMA antibody.

In one aspect, provided herein is a method of enriching a PSMA-expressing cell comprising: contacting a PSMA-expressing cell with a PSMA antibody provided herein; and enriching the PSMA-expressing cell bound to the PSMA antibody. In one aspect, provided herein is a method of isolating a PSMA-expressing cell comprising: contacting a PSMA-expressing cell with a PSMA antibody provided herein; and isolating the PSMA-expressing cell bound to the PSMA antibody. In one aspect, provided herein is a method of separating a PSMA-expressing cell comprising: contacting a PSMA-expressing cell with a PSMA antibody provided herein; and separating the PSMA-expressing cell bound to the PSMA antibody. In one aspect, provided herein is a method of purifying a PSMA-expressing cell comprising: contacting a PSMA-expressing cell with a PSMA antibody provided herein; and purifying the PSMA-expressing cell bound to the PSMA antibody. In one aspect, provided herein is a method of sorting a PSMA-expressing cell comprising: contacting a PSMA-expressing cell with a PSMA antibody provided herein; and sorting the PSMA-expressing cell bound to the PSMA antibody. In one aspect, provided herein is a method of selecting a PSMA-expressing cell comprising: contacting a PSMA-expressing cell with a PSMA antibody provided herein; and selecting the PSMA-expressing cell bound to the PSMA antibody. In one aspect, provided herein is a method of capturing a PSMA-expressing cell comprising: contacting a PSMA-expressing cell with a PSMA antibody provided herein; and capturing the PSMA-expressing cell bound to the PSMA antibody. In one aspect, provided herein is a method of detecting a PSMA-expressing cell comprising: contacting a PSMA-expressing cell with a PSMA antibody provided herein; and detecting the PSMA-expressing cell bound to the PSMA antibody.

In one aspect, provided herein is a method of enriching a PSMA-expressing cell comprising: contacting a PSMA-expressing cell with a PSMA antibody provided herein; and enriching the PSMA-expressing cell based on binding of the PSMA-expressing cell to the PSMA antibody. In one aspect, provided herein is a method of isolating a PSMA-expressing cell comprising: contacting a PSMA-expressing cell with a PSMA antibody provided herein; and isolating the PSMA-expressing cell based on binding of the PSMA-expressing cell to the PSMA antibody. In one aspect, provided herein is a method of separating a PSMA-expressing cell comprising: contacting a PSMA-expressing cell with a PSMA antibody provided herein; and separating the PSMA-expressing cell based on binding of the PSMA-expressing cell to the PSMA antibody. In one aspect, provided herein is a method of purifying a PSMA-expressing cell comprising: contacting a PSMA-expressing cell with a PSMA antibody provided herein; and purifying the PSMA-expressing cell based on binding of the PSMA-expressing cell to the PSMA antibody. In one aspect, provided herein is a method of sorting a PSMA-expressing cell comprising: contacting a PSMA-expressing cell with a PSMA antibody provided herein; and sorting the PSMA-expressing cell based on binding of the PSMA-expressing cell to the PSMA antibody. In one aspect, provided herein is a method of selecting a PSMA-expressing cell comprising: contacting a PSMA-expressing cell with a PSMA antibody provided herein; and selecting the PSMA-expressing cell based on binding of the PSMA-expressing cell to the PSMA antibody. In one aspect, provided herein is a method of capturing a PSMA-expressing cell comprising: contacting a PSMA-expressing cell with a PSMA antibody provided herein; and capturing the PSMA-expressing cell based on binding of the PSMA-expressing cell to the PSMA antibody. In one aspect, provided herein is a method of detecting a PSMA-expressing cell comprising: contacting a PSMA-expressing cell with a PSMA antibody provided herein; and detecting the PSMA-expressing cell based on binding of the PSMA-expressing cell to the PSMA antibody.

In certain embodiments of the methods, the PSMA-expressing cell is a prostate cell. In some embodiments of the methods, the PSMA-expressing cell is in a population of cells. In some embodiments of the methods, the PSMA-expressing cell is in a population of prostate cells. In some embodiments of the methods, the PSMA-expressing cell is in a population of cancer cells. In some embodiments of the methods, the PSMA-expressing cell is provided as a population of cells. In some embodiments of the methods, the PSMA-expressing cell is provided as a population of prostate cells. In some embodiments of the methods, the PSMA-expressing cell is provided as a population of cancer cells. In some embodiments of the methods, the PSMA-expressing cell is provided as a sample comprising a population of cells. In some embodiments of the methods, the PSMA-expressing cell is provided as a sample comprising a population of prostate cells. In some embodiments of the methods, the PSMA-expressing cell is provided as a sample comprising a population of cancer cells. In some embodiments of the methods, the sample is a blood sample. In some embodiments of the methods, the sample is a tissue sample. In some embodiments of the methods, the sample is a tissue culture sample.

Enrichment, isolation, separation, purification, sorting, selecting, capturing or detecting, or any combination thereof can be done using known technologies such as bead, microfluidics, solid support, columns, and the like. For example, PSMA cells can be separated or visualized using known methods when bound to the PSMA antibodies provided herein.

In some embodiments of the methods, the PSMA antibody is a multispecific PSMA antibody provided herein. In some embodiments of the methods, the PSMA antibody is a bispecific PSMA antibody provided herein. In some embodiments of the methods, the PSMA antibody is a trispecific PSMA antibody provided herein. In some embodiments of the methods, the PSMA antibody is a quadraspecific PSMA antibody provided herein. In certain embodiments, the PSMA antibody specifically binds to PSMA. In one embodiment, the multispecific PSMA antibody comprises: (a) a first binding domain that binds PSMA, and (b) a second binding domain that binds to a second target. In one embodiment, the multispecific PSMA antibody comprises: (a) a first binding domain that binds PSMA, and (b) a second binding domain that binds to a second target, and (c) a third binding domain that binds to a third target. In one embodiment, the multispecific PSMA antibody comprises: (a) a first binding domain that binds PSMA, and (b) a second binding domain that binds to a second target, (c) a third binding domain that binds to a third target, and (d) a fourth binding domain that binds to a fourth target. In one embodiment, the multispecific PSMA antibody comprises: (a) a first binding domain that specifically binds PSMA, and (b) a second binding domain that specifically binds to a second target. In one embodiment, the multispecific PSMA antibody comprises: (a) a first binding domain that specifically binds PSMA, and (b) a second binding domain that specifically binds to a second target, and (c) a third binding domain that specifically binds to a third target. In one embodiment, the multispecific PSMA antibody comprises: (a) a first binding domain that specifically binds PSMA, and (b) a second binding domain that specifically binds to a second target, (c) a third binding domain that specifically binds to a third target, and (d) a fourth binding domain that specifically binds to a fourth target.

In some embodiments of the methods, the PSMA antibody is a multispecific PSMA antibody, wherein the second target is CD3.

In some embodiments, the first binding domain of the multispecific PSMA antibody specifically binds PSMA. In some embodiments, the first binding domain of the multispecific PSMA antibody specifically binds to Pan troglodytes (chimpanzee, chimp) PSMA. In other embodiments, the first binding domain of the multispecific PSMA antibody specifically binds to Macaca fascicularis (cynomolgus monkey, macaque, cyno) PSMA. In yet other embodiments, the first binding domain of the multispecific PSMA antibody specifically binds to and/or human PSMA. In some embodiments, the first binding domain of the multispecific PSMA antibody specifically binds to Pan troglodytes, Macaca fascicularis, and human PSMA. In specific embodiments, the first binding domain of the multispecific PSMA antibody specifically binds to both cyno and human PSMA.

In specific embodiments of the methods provided herein, the method uses multi-marker detection. In some embodiments, the multi-marker detection uses a multispecific PSMA antibody provided herein. In some embodiments, the multi-marker detection uses a bispecific PSMA antibody provided herein. In some embodiments, the multi-marker detection uses a trispecific PSMA antibody provided herein. In some embodiments, the multi-marker detection uses a quadraspecific PSMA antibody provided herein.

In certain embodiments of the methods provided herein, the methods are included as steps in a diagnostic method. In certain embodiments of the methods provided herein, the methods are included as steps in a method to quantify the PSMA-expressing T cells.

In certain embodiments of the methods provided herein, the method further comprises expanding the enriched, isolated, separated, purified, sorted, selected, captured or detected PSMA-expressing cells. In certain embodiments, the expanding is in vitro. In certain embodiments, the expanding is in vivo. In certain embodiments of the methods provided herein, the method further comprises growing the enriched, isolated, separated, purified, sorted, selected, captured or detected PSMA-expressing cells. In certain embodiments, the growing is in vitro. In certain embodiments, the growing is in vivo. In certain embodiments of the methods provided herein, the method further comprises quantifying the enriched, isolated, separated, purified, sorted, selected, captured or detected PSMA-expressing cells.

Kits

Also provided are kits comprising a PSMA antibody provided herein, and instructions for use.

The described kits can be used to carry out the methods of using the PSMA-specific antibodies or antigen-binding fragments provided herein, or other methods known to those skilled in the art. In some embodiments, the described kits can include the antibodies or antigen-binding fragments described herein and reagents for use in detecting the presence of PSMA in a biological sample. Accordingly, the described kits can include one or more of the antibodies, or an antigen-binding fragment(s) thereof, described herein and a vessel for containing the antibody or fragment when not in use, instructions for use of the antibody or fragment, the antibody or fragment affixed to a solid support, and/or detectably labeled forms of the antibody or fragment, as described herein.

In one embodiment, provides is a kit comprising a PSMA antibody provided herein.

In another embodiment, provided is a kit comprising the bispecific PSMAxCD3 antibody comprising a first domain specifically binding PSMA and a second domain specifically binding CD3 provided herein.

The kit can be used for therapeutic uses and as diagnostic kits. The kit can also be used to detect the presence of PSMA, CD3 or PSMA and CD3 in a biological sample.

In some embodiments, the kit comprises an antibody described herein and reagents for detecting the antibody. The kit can further include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antibody provided herein in a container and instructions for use of the kit.

In some embodiments, the antibody in the kit is labeled.

### Embodiments

This invention provides the following non-limiting embodiments:
1. An isolated antibody that binds to PSMA comprising:
   (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32;
   (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66;
   (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
   (4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
   (5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
   (6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
   (7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
   (8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
   (9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
   (10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
   (11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372;
   (12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406;
   (13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
   (14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
   (15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
   (16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542;
   (17) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576;
   (18) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
   (19) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
   (20) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678;
   (21) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
   (22) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746;
   (23) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780; or
   (24) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814.
2. The isolated antibody of embodiment 1, wherein the VH CDR1, VH CDR2, VH CDR3, VL CDR1, VL CDR2, and VL CDR3 amino acid sequences are according to the
   (i) Kabat numbering system; (ii) Chothia numbering system;
   (iii) AbM numbering system;
   (iv) Contact numbering system;
   (v) IMGT numbering system or a combination thereof
3. The isolated antibody of embodiment 1 or embodiment 2, further comprising a VH having an amino acid sequence at least 80% identical to the amino acid sequence of SEQ ID NOs:31, 99, 167, 235, 303, 371, 405, 439, 473, 507, 541, 575, 643, 677, 711, 779 or 813 and a VL having an amino acid sequence at least 80% identical to the amino acid sequence of SEQ ID NOs:32, 66, 100, 134, 236, 270, 372, 406, 474, 508, 542, 576, 678, 746, 780 or 814.
4. The isolated antibody of any one of embodiments 1 to 3, further comprising a heavy chain (HC) having an amino acid sequence at least 80% identical to the amino acid sequence of SEQ ID NOs:33, 101, 169, 237, 305, 373, 407 or 441 and a light chain (LC) having an amino acid sequence at least 80% identical to the amino acid sequence of SEQ ID NOs:34, 68, 102, 136, 238, 272, 374 or 408.
5. An isolated antibody that binds PSMA comprising
   a. (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
   b. (i) a VH comprising a VH CDR1, a VH CDR2, and a VH CDR3 having an amino acid sequence of SEQ ID NOs:205, 206, and 411, respectively, and (ii) a VL comprising a VL CDR1, VL CDR2, and VL CDR3 having an amino acid sequence of SEQ ID NOs:242, 209, and 244, respectively;
   c. (i) a VH having an amino acid sequence of SEQ ID NO:439; and (ii) a VL having an amino acid sequence of SEQ ID NO:270; or
   d. (i) a HC having an amino acid sequence of SEQ ID NO:441; and (ii) a LC having an amino acid sequence of SEQ ID NO:272.
6. The isolated antibody of any one of embodiments 1 to 5, wherein the antibody is a humanized antibody.
7. The isolated antibody of any one of embodiments 1 to 6, wherein the antibody is a human antibody.
8. The isolated antibody of any one of embodiments 1 to 7, wherein the binding domain that binds to PSMA is a scFv, an scFv dimer, a Fv, a Fab, a Fab, a F(ab′)₂, a dsFv, a sdAb, a VHH or a single chain antibody.
9. The isolated antibody of any one of embodiments 1 to 8, wherein the antibody is an IgG antibody.
10. The isolated antibody of embodiment 9, wherein the IgG antibody is an IgG1, IgG2, IgG3, or IgG4 antibody.
11. The isolated antibody of any one of embodiments 1 to 10, wherein the antibody comprises a kappa light chain.
12. The isolated antibody of any one of embodiments 1 to 10, wherein the antibody comprises a lambda light chain.
13. The isolated antibody of any one of embodiments 1 to 12, wherein the antibody is a monoclonal antibody.
14. The isolated antibody of any one of embodiments 1 to 13, wherein the antibody binds a PSMA antigen.
15. The isolated antibody of any one of embodiments 1 to 14, wherein the antibody binds a PSMA epitope.
16. The isolated antibody of any one of embodiments 1 to 15, wherein the antibody specifically binds to PSMA.
17. The isolated antibody of any one of embodiments 1 to 16, wherein the VH CDR1, VH CDR2, VH CDR3, VL CDR1, VL CDR2 and VL CDR3 form a binding site for an antigen of the PSMA.
18. The isolated antibody of any one of embodiments 1 to 16, wherein the VH CDR1, VH CDR2, VH CDR3, VL CDR1, VL CDR2 and VL CDR3 form a binding site for an epitope of the PSMA.
19. The isolated antibody of any one of embodiments 1 to 18, wherein the PSMA is present on the surface of a cell.
20. The isolated antibody of embodiment 19, wherein the cell is a prostate cell.
21. The isolated antibody of embodiment 19, wherein the cell is a prostate cancer cell.
22. The isolated antibody of embodiment 19, wherein the cell is a renal cell.
23. The isolated antibody of embodiment 19, wherein the cell is a renal cancer cell.
24. The isolated antibody of embodiment 23, wherein the renal cancer is a metastatic renal cell carcinoma.
25. The isolated antibody of any one of embodiments 1 to 24, wherein the antibody is multivalent.
26. The isolated antibody of embodiment 25, wherein the antibody is capable of binding at least three antigens.
27. The isolated antibody of embodiment 25, wherein the antibody is capable of binding at least four antigens.
28. The isolated antibody of embodiment 25, wherein the antibody is capable of binding at least five antigens.
29. The isolated antibody of any one of embodiments 1 to 28, wherein the antibody is a multispecific antibody.
30. The isolated antibody of embodiment 29, wherein the antibody is a bispecific antibody.
31. The isolated antibody of embodiment 29, wherein the antibody is a trispecific antibody.
32. The isolated antibody of embodiment 29, wherein the antibody is a quadraspecific antibody.
33. A nucleic acid encoding the antibody of any one of embodiments 1 to 32.
34. A vector comprising the nucleic acid of embodiment 33.
35. A host cell comprising the vector of embodiment 34.
36. A kit comprising the vector of embodiment 34 and packaging for the same.
37. A kit comprising the antibody of any one of embodiments 1 to 32 and packaging for the same.
38. A pharmaceutical composition comprising the antibody of any one of embodiments 1 to 32, and a pharmaceutically acceptable carrier.
39. A method of producing the pharmaceutical composition of embodiment 38, comprising combining the antibody with a pharmaceutically acceptable carrier to obtain the pharmaceutical composition.
40. An isolated multispecific antibody, comprising:
   (a) a first binding domain that binds to PSMA, wherein the first binding domain comprises:
      (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32;
      (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66;
      (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
      (4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
      (5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
      (6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
      (7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
      (8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
      (9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
      (10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
      (11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372;
      (12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406;
      (13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
      (14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
      (15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
      (16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542;
      (17) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576;
      (18) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
      (19) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
      (20) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678;
      (21) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
      (22) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746;
      (23) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780; or
      (24) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814; and
   (b) a second binding domain that binds to CD3, wherein the second binding domain comprises:
      (1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1505;
         and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1464;
      (2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916;
      (3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984;
      (4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1463;
         and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1464;
      (5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848; or
      (6) a scFv comprising a VH CDR1, a VH CDR2, a VH CDR3, a VL CDR1, a VL CDR2, a VL CDR3 having an amino acid sequence of a VH CDR1, a VH CDR2, a VH CDR3, a VL CDR1, a VL CDR2, a VL CDR3 respectively, of a scFv having an amino acid sequence of SEQ ID NO:1524.
41. The isolated multispecific antibody of embodiment 40, wherein the VH CDR1, VH CDR2, VH CDR3, VL CDR1, VL CDR2, and VL CDR3 amino acid sequences are according to the
   (i) Kabat numbering system; (ii) Chothia numbering system;
   (iii) AbM numbering system;
   (iv) Contact numbering system;
   (v) IMGT numbering system or a combination thereof
42. The isolated multispecific antibody of embodiment 40 or embodiment 41, wherein the first binding domain that binds PSMA comprises a heavy chain (HC) having an amino acid sequence at least 80% identical to the amino acid sequence of SEQ ID NOs:33, 101, 169, 237, 305, 373, 407, 441, 1242, 1244, 1248, 1250, 1252, 1254, 1256, 1258, 1260, 1262, 1264, 1266, 1268 or 1270 and/or a light chain (LC) having an amino acid sequence at least 80% identical to the amino acid sequence of SEQ ID NOs:34, 68, 102, 136, 238, 272, 374 or 408.
43. The isolated multispecific antibody of any one of embodiments 40 to 42, wherein the first binding domain that binds PSMA comprises a scFv having an amino acid sequence at least 80% identical to the amino acid sequence of any one of SEQ ID NOs:1485-1500 or SEQ ID NOs:1526-1531.
44. The isolated multispecific antibody of any one of embodiments 40 to 43, wherein the first binding domain that binds PSMA comprises (i) a HC having an amino acid sequence of SEQ ID NO:441; and (ii) a LC having an amino acid sequence of SEQ ID NO:272.
45. The isolated multispecific antibody of any one of embodiments 40 to 44, wherein the second binding domain that binds CD3 comprises a heavy chain (HC) having an amino acid sequence at least 80% identical to the amino acid sequence of SEQ ID NOs:849, 883, 917, 951, 985, 1019, 1504, 1455, 1192, 1194, 1167, 1218 or 1238 and/or a light chain (LC) having an amino acid sequence at least 80% identical to the amino acid sequence of SEQ ID NOs:850, 918, 986, 1193, 1195 or 1219.
46. The isolated multispecific antibody of any one of embodiments 40 to 45, wherein the second binding domain that binds CD3 comprises a scFv having an amino acid sequence at least 80% identical to the amino acid sequence of SEQ ID NOs:1186, 1187, 1523 or 1524.
47. An isolated bispecific antibody comprising a first binding domain that binds PSMA and a second binding domain that binds CD3, wherein
   a. the first binding domain that binds PSMA comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270, and the second binding domain that binds CD3 comprises a scFv comprising a VH CDR1, a VH CDR2, a VH CDR3, a VL CDR1, a VL CDR2, and a VL CDR3 of a scFv having an amino acid sequence of SEQ ID NO:1524;
   b. the first binding domain that binds PSMA comprises a VH CDR1, VH CDR2, VH CDR3, VL CDR1, VL CDR2, VL CDR3 of SEQ ID NO:205, 206, 411, 242, 209 and 244, respectively, wherein the amino acid sequences are according to the Kabat numbering system; and the second binding domain that binds CD3 comprises a VH CDR1, VH CDR2, VH CDR3, VL CDR1, VL CDR2, VL CDR3 of SEQ ID NO:1467, 1468, 1506, 1470, 1471 and 1472, respectively; wherein the amino acid sequences are according to the Kabat numbering system;
   c. the first binding domain that binds PSMA comprises (i) a VH having an amino acid sequence of SEQ ID NO:439; and (ii) a VL having an amino acid sequence of SEQ ID NO:270, and the second binding domain that binds CD3 comprises a scFv of SEQ ID NO:1524, respectively; and/or
   d. the first binding domain that binds PSMA comprises (i) a HC2 having an amino acid sequence of SEQ ID NO:441; and (ii) a LC2 having an amino acid sequence of SEQ ID NO:272, respectively; and the second binding domain that binds CD3 comprises a HC1 of SEQ ID NO:1455.
48. The isolated multispecific antibody of any one of embodiments 40 to 47, wherein the antibody is a humanized antibody.
49. The isolated multispecific antibody of any one of embodiments 40 to 48, wherein the antibody is a human antibody.
50. The isolated multispecific antibody of any one of embodiments 40 to 49, wherein the first binding domain, the second binding domain and/or the first and second is a scFv, an scFv dimer, a Fv, a Fab, a Fab, a F(ab)₂, a dsFv, a sdAb, a VHH or a single chain antibody.
51. The isolated multispecific antibody of any one of embodiments 40 to 50, wherein the antibody is an IgG antibody.
52. The isolated multispecific antibody of embodiment 51, wherein the IgG antibody is an IgG1, IgG2, IgG3, or IgG4 antibody.
53. The isolated multispecific antibody of any one of embodiments 40 to 52, wherein the antibody comprises a kappa light chain.
54. The isolated multispecific antibody of any one of embodiments 40 to 52, wherein the antibody comprises a lambda light chain.
55. The isolated multispecific antibody of any one of embodiments 40 to 54, wherein the antibody is a monoclonal antibody.
56. The isolated multispecific antibody of any one of embodiments 40 to 55, wherein the first binding domain binds a PSMA antigen.
57. The isolated multispecific antibody of any one of embodiments 40 to 55, wherein first binding domain binds a PSMA epitope.
58. The isolated multispecific antibody of any one of embodiments 40 to 57, wherein the first binding domain specifically binds to PSMA.
59. The isolated multispecific antibody of any one of embodiments 40 to 58, wherein the VH CDR1, VH CDR2, VH CDR3, VL CDR1, VL CDR2 and VL CDR3 of the first binding domain form a binding site for an antigen of the PSMA.
60. The isolated multispecific antibody of any one of embodiments 40 to 58, wherein the VH CDR1, VH CDR2, VH CDR3, VL CDR1, VL CDR2 and VL CDR3 of the first binding domain form a binding site for an epitope of the PSMA.
61. The isolated multispecific antibody of any one of embodiments 40 to 60, wherein the second binding domain binds a CD3 antigen.
62. The isolated multispecific antibody of any one of embodiments 40 to 61, wherein the second binding domain binds a CD3 epitope.
63. The isolated multispecific antibody of any one of embodiments 40 to 62, wherein the second binding domain specifically binds to CD3.
64. The isolated multispecific antibody of any one of embodiments 40 to 63, wherein the VH CDR1, VH CDR2, VH CDR3, VL CDR1, VL CDR2 and VL CDR3 of the second binding domain form a binding site for an antigen of the CD3.
65. The isolated multispecific antibody of any one of embodiments 40 to 63, wherein the VH CDR1, VH CDR2, VH CDR3, VL CDR1, VL CDR2 and VL CDR3 of the second binding domain form a binding site for an epitope of the CD3.
66. The isolated multispecific antibody of any one of embodiments 40 to 65, wherein the PSMA is present on the surface of a cell.
67. The isolated multispecific antibody of embodiment 66, wherein the cell is a prostate cell.
68. The isolated multispecific antibody of embodiment 66, wherein the cell is a prostate cancer cell.
69. The isolated multispecific antibody of embodiment 66, wherein the cell is a renal cell.
70. The isolated multispecific antibody of embodiment 66, wherein the cell is a renal cancer cell.
71. The isolated multispecific antibody of embodiment 66, wherein the renal cancer is a metastatic renal cell carcinoma.
72. The isolated multispecific antibody of any one of embodiments 40 to 71, wherein the antibody is a bispecific antibody.
73. The isolated multispecific antibody of any one of embodiments 40 to 71, wherein the antibody is a trispecific antibody.
74. The isolated multispecific antibody of any one of embodiments 40 to 71, wherein the antibody is a quadraspecific antibody.
75. A nucleic acid encoding the multispecific antibody of any one of embodiments 40 to 74.
76. A vector comprising the nucleic acid of embodiment 75.
77. A host cell comprising the vector of embodiment 76.
78. A kit comprising the vector of embodiment 76 and packaging for the same.
79. A kit comprising the multispecific antibody of any one of embodiments 40 to 74 and packaging for the same.
80. A pharmaceutical composition comprising the multispecific antibody of any one of embodiments 40 to 74, and a pharmaceutically acceptable carrier.
81. A method of producing the pharmaceutical composition of embodiment 80, comprising combining the multispecific antibody with a pharmaceutically acceptable carrier to obtain the pharmaceutical composition.
82. A method of directing a CD3-expressing T cell to a PSMA-expressing target cell, comprising contacting the T cell with the multispecific antibody of any one of embodiments 40 to 74.
83. The method of embodiment 82, wherein the contacting directs the T cell to the target cell.
84. A method of inhibiting the growth or proliferation of a PSMA-expressing target cell, comprising contacting the target cell with the multispecific antibody of any one of embodiments 40 to 74.
85. The method of embodiment 84, wherein the contacting is in the presence of CD3-expressing T cells.
86. A method of eliminating a PSMA-expressing target cell in a subject, comprising administering to the subject an effective amount of the multispecific antibody of any one of embodiments 40 to 74.
87. The method of embodiment 86, wherein the subject has a disease or disorder of the prostate.
88. The method of embodiment 86, wherein the subject has prostate inflammation.
89. The method of embodiment 86, wherein the subject has Benign prostatic hyperplasia.
90. The method of embodiment 86, wherein the subject has a prostate cancer.
91. The method of embodiment 90, wherein the subject has metastatic castration-resistant prostate cancer (mCRPC).
92. The method of any one of embodiments 86 to 91, wherein the target cell expresses PSMA on the cell surface.
93. The method of any one of embodiments 86 to 91, wherein the target cell is a prostate cell.
94. The method of any one of embodiments 86 to 91, wherein the target cell is a prostate cancer cell.
95. The method of embodiment 86, wherein the subject has a renal disease or disorder.
96. The method of embodiment 86, wherein the subject has a renal cancer.
97. The method of embodiment 86, wherein the subject has a metastatic renal cell carcinoma.
98. The method of any one of embodiments 86 or 95 to 97, wherein the target cell expresses PSMA on the cell surface.
99. The method of any one of embodiments 86 or 95 to 97, wherein the target cell is a renal cell.
100. The method of any one of embodiments 86 or 95 to 97, wherein the target cell is a renal cancer cell.
101. The method of any one of embodiments 86 or 95 to 97, wherein the target cell is a metastatic renal cell carcinoma cell.
102. The method of any one of embodiments 86 to 101, wherein the subject is a subject in need thereof
103. A method of treating a disease or disorder in a subject, comprising administering to the subject an effective amount of the multispecific antibody of any one of embodiments 40 to 74.
104. The method of embodiment 103, wherein the disease or disorder is a disease or disorder of the prostate.
105. The method of embodiment 103, wherein the disease or disorder of the prostate is prostate inflammation.
106. The method of embodiment 103, wherein the disease or disorder of the prostate is Benign prostatic hyperplasia.
107. The method of embodiment 103, wherein the disease or disorder of the prostate is a prostate cancer.
108. The method of embodiment 107, wherein the disease or disorder is metastatic castration-resistant prostate cancer (mCRPC).
109. The method of embodiment 103, wherein the disease or disorder is a renal disease or disorder.
110. The method of embodiment 103, wherein the renal disease or disorder is a renal cancer.
111. The method of embodiment 103, wherein the renal disease or disorder is a metastatic renal cell carcinoma.
112. The method of any one of embodiments 103 to 111, wherein the subject is a subject in need thereof.

Provided in the Examples herein are exemplary antibodies that bind to PSMA. Also provided in the Examples herein are exemplary bispecific antibodies that bind to PSMA and CD3.

In some embodiments, provided herein is a bispecific antibody comprising: (a) a first binding domain that binds to a PSMA antigen, and (b) a second binding domain that binds to a second target antigen. In some embodiments, provided herein is a bispecific antibody comprising: (a) a first binding domain that specifically binds to a PSMA antigen, and (b) a second binding domain that specifically binds to a second target antigen. In some embodiments, provided herein is a bispecific antibody comprising: (a) a first binding domain that binds to a first epitope on a PSMA antigen, and (b) a second binding domain that binds to a second epitope on a second target antigen. In some embodiments, provided herein is a bispecific antibody comprising: (a) a first binding domain that specifically binds to a first epitope on a PSMA antigen, and (b) a second binding domain that specifically binds to a second epitope on a second target antigen. In certain embodiments, the second target antigen is CD3.

Exemplary binding agents that bind to PSMA are provided herein, for example in the Examples, as well as Tables 4-12.

Exemplary binding agents (e.g., multispecific PSMA antibodies) can comprise a second binding domain that binds to CD3, such as those provided in Tables 16-22.

In addition, exemplary binding agents that bind to PSMA and CD3 are provided elsewhere herein, for example in the Examples, as well as Tables 23-28.

Particular embodiments of this invention are described herein. Upon reading the foregoing description, variations of the disclosed embodiments can become apparent to individuals working in the art, and it is expected that those skilled artisans can employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications can be made without departing from the spirit and scope of the invention. Accordingly, the descriptions in the Examples section are intended to illustrate but not limit the scope of invention described in the claims.

### EXAMPLES

The following examples are based on the premise that bispecific anti-PSMA/anti-CD3 antibodies, or antigen-binding fragments thereof, have potent anti-tumor functions. Prostate tumor cells express prostate specific membrane antigen (PSMA), a type II membrane protein that is highly expressed in prostatic intraepithelial neoplasia (PIN), and aggressive prostate cancers. CD3 is expressed on T cells and is involved in T cell activation and differentiation into an effector cell. This ability of CD3 to activate T cells is harnessed using bispecific antibodies constructed such that one arm binds to the PSMA, and the other arm binds to CD3 on the T cells. Thus, the bispecific antibody bridges together effector and target cells, resulting in tumor killing.

### Example 1: Antigen Generation

Human PSMA extracellular domain (ECD) was produced based on Uniprot Accession #Q04609 sequence. The ECD construct was designed with a 6×His-tag sequence (SEQ ID NO:1484) at the N-terminus (construct PSMW39; SEQ ID NO:1023). The PSMA ECD from Macaca fascicularis was produced based on NCBI Accession #EHH56646.1, appending the sequence at the N-terminus with an Avi-tag fused to a 6× polyhistidine tag (SEQ ID NO:1484) (construct PSMW1; SEQ ID NO:1024). The extracellular domain (ECD) of PSMA from Mus musculus was produced based on Uniprot Accession #035409, appending the ECD sequence at the N-terminus with an Avi-tag fused to a 6× polyhistidine tag (SEQ ID NO:1484) for purification (construct PSMW29; SEQ ID NO:1025). The cynomolgus and murine ECD expression constructs were used to transiently transfect HEK293-6E. Cells were incubated for six days at 37° C. with 5% CO₂ prior to harvest. The human ECD expression construct was used to stably transfect Expi-CHO using EXPIFECTAMINE-CHO. Stable transfectants were selected with G418 (Thermo Fisher Scientific; Cat #10131027). Monoclones expressing the highest levels of human PSMA ECD protein were identified by ELISA. High PSMW39-expressing monoclones were cultured in DYNAMIS media supplemented with 5% Cell Boost 5 and incubated at 37° C., 125 rpm and 5% CO₂ until the cell viability dropped below 80%. On days 2, 4, 7, 9 and 11, 6 mL of 20% D-Glucose (2 g/L final conc.) and 6 mL of 200 mM L-Glutamine (2 mM final conc.) was added as feed to each of the flasks. For the purification of all recombinant PSMA proteins, the expressed cells were removed by centrifugation and the soluble PSMA proteins with his-tags were purified from the media using immobilized metal affinity chromatography using His 60 Ni SUPERFLOW Resin, Clonetech, Cat #635662 followed by SUPERDEX 200 preparative size exclusion chromatography (SEC) (GE Healthcare) and formulation into 1×DPBS, pH 7.2 containing 1 mM CaCl₂, 0.5 mM MgCl₂, and 0.5 mM ZnCl₂. The isolation of the homodimeric species was confirmed via analytical size exclusion chromatography. The amino acid sequences of the recombinant antigens are shown in Table 3.

TABLE 3
The amino acid sequences of the
recombinant PSMA antigens.

| Protein | | |
|---|---|---|
| AA ID | Description | Amino Acid Sequence |
| PSMW39 | Human | HHHHHHKSSNEATNITPKHN |
| | PSMA ECD | MKAFLDELKAENIKKFLYNF |
| | (Lys44- | TQIPHLAGTEQNFQLAKQIQ |
| | Ala750), | SQWKEFGLDSVELAHYDVLL |
| | N-6xHis | SYPNKTHPNYISIINEDGNE |
| | | IFNTSLFEPPPPGYENVSDI |
| | | VPPFSAFSPQGMPEGDLVYV |
| | | NYARTEDFFKLERDMKINCS |
| | | GKIVIARYGKVFRGNKVKNA |
| | | QLAGAKGVILYSDPADYFAP |
| | | GVKSYPDGWNLPGGGVQRGN |
| | | ILNLNGAGDPLTPGYPANEY |
| | | AYRRGIAEAVGLPSIPVHPI |
| | | GYYDAQKLLEKMGGSAPPDS |
| | | SWRGSLKVPYNVGPGFTGNF |
| | | STQKVKMHIHSTNEVTRIYN |
| | | VIGTLRGAVEPDRYVILGGH |
| | | RDSWVEGGIDPQSGAAVVHE |
| | | IVRSFGTLKKEGWRPRRTIL |
| | | FASWDAEEFGLLGSTEWAEE |
| | | NSRLLQERGVAYINADSSIE |
| | | GNYTLRVDCTPLMYSLVHNL |
| | | TKELKSPDEGFEGKSLYESW |
| | | TKKSPSPEFSGMPRISKLGS |
| | | GNDFEVEFQRLGIASGRARY |
| | | TKNWETNKFSGYPLYHSVYE |
| | | TYELVEKFYDPMFKYHLTVA |
| | | QVRGGMVFELANSIVLPFDC |
| | | RDYAVVLRKYADKIYSISMK |
| | | HPQEMKTYSVSFDSLESAVK |
| | | NFTEIASKFSERLQDFDKSN |
| | | PIVLRMMNDQLMFLERAFID |
| | | PLGLPDRPFYRHVIYAPSSH |
| | | NKYAGESFPGIYDALFDIES |
| | | KVDPSKAWGEVKRQIYVAAF |
| | | TVQAAAETLSEVA |
| | | (SEQ ID NO: 1023) |
| PSMW1 | Avi-6xHis- | GLNDIFEAQKIEWHEHHHHH |
| | GS-cyno | HGSKSSSEATNITPKHNMKA |
| | PSMA ECD | FLDELKAENIKKFLHNFTQI |
| | | PHLAGTEQNFQLAKQIQSQW |
| | | KEFGLDSVELTHYDVLLSYP |
| | | NKTHPNYISIINEDGNEIFN |
| | | TSLFEPPPAGYENVSDIVPP |
| | | FSAFSPQGMPEGDLVYVNYA |
| | | RTEDFFKLERDMKINCSGKI |
| | | VIARYGKVERGNKVKNAQLA |
| | | GATGVILYSDPDDYFAPGVK |
| | | SYPDGWNLPGGGVQRGNILN |
| | | LNGAGDPLTPGYPANEYAYR |
| | | RGMAEAVGLPSIPVHPIGYY |
| | | DAQKLLEKMGGSASPDSSWR |
| | | GSLKVPYNVGPGFTGNFSTQ |
| | | KVKMHIHSTSEVTRIYNVIG |
| | | TLRGAVEPDRYVILGGHRDS |
| | | WVEGGIDPQSGAAVVHEIVR |
| | | SFGMLKKEGWRPRRTILFAS |
| | | WDAEEFGLLGSTEWAEENSR |
| | | LLQERGVAYINADSSIEGNY |
| | | TLRVDCTPLMYSLVYNLTKE |
| | | LESPDEGFEGKSLYESWTKK |
| | | SPSPEFSGMPRISKLGSGND |
| | | FEVFFQRLGIASGRARYTKN |
| | | WETNKFSSYPLYHSVYETYE |
| | | LVEKFYDPMEKYHLTVAQVR |
| | | GGMVFELANSVVLPFDCRDY |
| | | AVVLRKYADKIYNISMKHPQ |
| | | EMKTYSVSFDSLFSAVKNFT |
| | | EIASKFSERLRDEDKSNPIL |
| | | LRMMNDQLMELERAFIDPLG |
| | | LPDRPFYRHVIYAPSSHNKY |
| | | AGESFPGIYDALFDIESKVD |
| | | PSQAWGEVKRQISIATFTVQ |
| | | AAAETLSEVA |
| | | (SEQ ID NO: 1024) |
| PSMW29 | Avi-6xHis- | GLNDIFEAQKIEWHEHHHHH |
| | GS-mouse | HGSKPSNEATGNVSHSGMKK |
| | PSMA (45- | EFLHELKAENIKKFLYNFTR |
| | 752) | TPHLAGTQNNFELAKQIHDQ |
| | | WKEFGLDLVELSHYDVLLSY |
| | | PNKTHPNYISIINEDGNEIF |
| | | KTSLSEQPPPGYENISDVVP |
| | | PYSAFSPQGTPEGDLVYVNY |
| | | ARTEDFFKLEREMKISCSGK |
| | | IVIARYGKVFRGNMVKNAQL |
| | | AGAKGMILYSDPADYFVPAV |
| | | KSYPDGWNLPGGGVQRGNVL |
| | | NLNGAGDPLTPGYPANEHAY |
| | | RHELTNAVGLPSIPVHPIGY |
| | | DDAQKLLEHMGGPAPPDSSW |
| | | KGGLKVPYNVGPGFAGNFST |
| | | QKVKMHIHSYTKVTRIYNVI |
| | | GTLKGALEPDRYVILGGHRD |
| | | AWVFGGIDPQSGAAVVHEIV |
| | | RSFGTLKKKGRRPRRTILFA |
| | | SWDAEEFGLLGSTEWAEEHS |
| | | RLLQERGVAYINADSSIEGN |
| | | YTLRVDCTPLMYSLVYNLTK |
| | | ELQSPDEGFEGKSLYDSWKE |
| | | KSPSPEFIGMPRISKLGSGN |
| | | DFEVFFQRLGIASGRARYTK |
| | | NWKTNKVSSYPLYHSVYETY |
| | | ELVVKFYDPTFKYHLTVAQV |
| | | RGAMVFELANSIVLPFDCQS |
| | | YAVALKKYADTIYNISMKHP |
| | | QEMKAYMISFDSLFSAVNNF |
| | | TDVASKFNQRLQELDKSNPI |
| | | LLRIMNDQLMYLERAFIDPL |
| | | GLPGRPFYRHIIYAPSSHNK |
| | | YAGESFPGIYDALFDISSKV |
| | | NASKAWNEVKRQISIATFTV |
| | | QAAAETLREVA |
| | | (SEQ ID NO: 1025) |

### Example 2—Generation of Anti-PSMA Antibodies

### Example 2.1: Antibody Generation Using Transgenic Ablexis® Mice

Ablexis® kappa, lambda, and kappa/lambda hybrid mice were immunized with recombinant human PSMA antigen (PSMW39.002) and cynomolgus PSMA antigen (PSMW1.009) in combination with CL413 adjuvant (InvivoGen, VAC-C413-5).

Ablexis® mice generate antibodies having human variable domains linked to human CH1 and CL domains, chimeric human/mouse hinge region, and mouse Fc regions. Ablexis® Kappa Mouse and Lambda Mouse strains are distinguished by which of their heavy chains are human or mouse as noted below. Antibodies produced by the Kappa Mouse lack sequence derived from mouse VH, DH and JH exons and mouse Vκ, Jκ and Cκ exons. The endogenous mouse Igλ is active in the Kappa Mouse. The human Igκ chains comprise approximately 90-95% of the naïve repertoire and mouse Igλ chains comprise approximately 5-10% of the naïve repertoire in this strain. Antibodies produced by the Lambda Mouse lack sequence derived from mouse VH, DH and JH exons and mouse Vλ, Jλ and Cλ exons. The endogenous mouse Igκ is active in the Lambda Mouse. The human Igλ chains comprise approximately 40% of the naïve repertoire and mouse Igκ chains comprise approximately 60% of the naïve repertoire. The preparation and use of Ablexis®, and the genomic modifications carried by such mice, is described in WO11/123708.

Mice were boosted at Days 0, 7, 14, 21, and 28 before being bled on Day 35 for serological analysis. Serology was performed on human PSMA (+) cells C4-2B (AG000002300) and a human PSMA knockout cell line (AG000002521). In total, 8 mice were selected for hybridoma fusion and final boosted on Day 56 with PSMW39.002 or an equimolar mixture of PSMW39.002 and PSMW1.009. This preparation also included recombinant anti-mouse CD40 mAb (R&D Systems, MAB440) to stimulate B cell expansion. On Day 60, spleen and draining lymph nodes were harvested from these mice, pooled and homogenized into a single cell suspension. Stable hybridomas were generated by PEG-mediated fusion of mouse myeloma cell line FO with the pooled mouse homogenate, followed by HAT selection.

Supernatants from these hybridomas were screened against C4-2B cells by MSD. From this primary screen, 440 positive samples were identified and re-arrayed for confirmatory screening. Confirmatory screening was performed by ELISA as well as Fluorescence-activated cell sorting (FACS) to validate binding to PSMA protein and PSMA (+) C4-2B cells, respectively. To ensure specificity, samples were also screened against an irrelevant negative control sample TfRW2. Based on the screening results, 214 samples were advanced past confirmatory screening from the 440 samples identified and isotyped for kappa or lambda light chain expression.

### Example 2.2: V Region Cloning

RNA from hybridoma were purified using RNEASY Plus Mini Kit (Qiagen) and used for cDNA synthesis using the SMARTER cDNA synthesis kit (Clontech, Mount View, CA). To facilitate cDNA synthesis, oligodT was used to prime reverse transcription of all messenger RNAs followed by “5′ capping” with a Smarter IIA oligonucleotide. Subsequent amplification of the VH and VL fragments was performed using a 2-step PCR amplification using 5′ primers targeting the Smarter IIA cap and 3′ primers targeting consensus regions in CH1. Briefly, each 50 μl PCR reaction consists of 20 μM of forward and reverse primer mixes, 25 μl of PRIMESTAR Max DNA polymerase premix (Clontech), 2 μl of unpurified cDNA, and 21 μl of double-distilled H₂O. The cycling program starts at 94° C. for 3 min, followed by 35 cycles (94° C. for 30 Sec, 55° C. for 1 min, 68° C. for 1 min), and ends at 72° C. for 7 min. The second round PCR was performed with VL and VH 2nd round primers containing 15 bp complementary extensions that “overlap” respective regions in their respective Lonza mother vector (VH and VL). Second round PCR was performed with the following program: 94° C. for 3 min; 35 cycles (94° C. for 30 Sec, 55° C. for 1 min, 68° C. for 1 min), and ends at 72° C. for 7 min. In-Fusion® HD Cloning Kit (Clonetech, U.S.A.) was used for directional cloning of VL gene into Lonza huIgK or Lambda vector and VH gene into Lonza huIgG1 vector. To facilitate In-Fusion® HD Cloning, PCR products were treated with Cloning Enhancer before In-Fusion HD Cloning. The resulting PCR fragments were sequenced. The amino-acid sequences of the recovered-regions were codon optimized and cloned into an expression vector carrying the IgG1 constant region with L234A, L235A and D265S mutations for Fc silencing (IgG1 AAS isotype), and the K409R mutation for heterodimerization or an expression vector carrying the IgG4 constant region with S228P, F234A and L235A mutations (IgG4PAA isotype). D365E mutation was also introduced. Cloning and transformation were performed according to manufacturer's protocol (Clonetech, U.S.A.). Mini-prep DNAs were subjected to Sanger sequencing to confirm that complete V-gene fragments were obtained.

### Example 2.3: scFv and scFv-Fc Formatting

Following cloning of the variable regions, the VH and VL sequences were used to generate antibodies. Antibodies were expressed in a Fab format, a mAb format, a scFv format in the VH-linker-VL orientation or a scFv format in VL-linker-VH orientation and were further analyzed as described below. The linker sequence GGSEGKSSGSGSESKSTGGS (SEQ ID NO:1419) was used to conjugate the VH/VL regions. A synthetic DNA fragment coding for the scFv moiety was designed using codons optimized for Cricetulus griseus (Chinese hamster) and was cloned between the Hindlll and PsiI sites within a mammalian expression vector containing the constant region.

### Example 2.4: ExpiCHO Transfection and Purification

PSMB890 identified from the immunization campaigns was cloned and expressed as IgG4PAA and purified. PSMB891, PSMB892, PSMB893, PSMB894, PSMB895, PSMB896, PSMB897, PSMB898 and PSMB899 were cloned and expressed as IgG1AAS and purified. ExpiCHO-S™ cells (ThermoFisher Scientific) were seeded at 1.25×10⁵-2.25×10⁵ viable cells/mL density in ExpiCHO™ Expression Medium and cultured in a 37° C., 7% CO₂ shaker incubator. For routine cell growth in 125 mL-2 L shake flasks; the shake speed was set to 130 rpm. Cells were sub-cultured when density reached log phase growth at 4×10⁶-6×10⁶ viable cells/mL with a 98-99% viability. Two days before transfection, ExpiCHO-S™ cells were seeded at 1.5×10⁶ viable cells/mL density. On day of transfection, the viable cell density and percent viability is determined. Cells were transfected at a density of 6×10⁶ viable cells/mL. For optimal transfection, sterile Heavy and Light Chain plasmid DNA at ≥1 mg/mL concentration in TE buffer (10 mM Tris-HCl, 1 mM EDTA, pH 8.0) were used. ExpiCHO-S™ cells were transfected following manufacturer's Max Titer Transfection protocol. All amounts and volumes shown below are per mL of final transfected culture volume. Briefly, plasmid DNA were diluted with 0.04 mL cold OptiPRO™ medium at the following ratio: 0.125 pg Heavy Chain DNA: 0.375 pg Light Chain DNA: 0.5 μg pAdvantage. 6.4 μL of ExpiFectamine™ CHO Transfection Reagent was diluted and mixed gently with 0.04 mL cold OptiPRO™ medium and incubated for 1 min. The diluted ExpiFectamine™ CHO Reagent was added to the diluted DNA, mixed gently and the ExpiFectamine™ CHO/plasmid DNA complexes were incubated at room temperature for 5 minutes. Post-incubation, the complexes were added to the ExpiCHO-S™ cells in a shaker flask and incubated overnight in a 37° C., 7% CO₂ shaker incubator. On Day 1 post-transfection, 6 μL ExpiFectamine™ CHO Enhancer and 160 μL ExpiCHO™ Feed were added and the flask was transferred to a 32° C., 7% CO₂ shaker incubator. Culture was harvested on Day 7 post-transfection, centrifuged at 850×g for 15 mins and clarified through a 0.2 μm Acropak 1500 filter capsule (Pall).

### Example 2.5: Purification of Anti-PSMA Antibodies

Antibodies were purified from the clarified supernatants using MABSELECT SURE Protein A columns equilibrated with 1×D-PBS, pH 7.2 prior. Unbound proteins were removed by washing extensively with 1×D-PBS, pH 7.2. Bound protein was eluted with 0.1 M Na-acetate, pH 3.5. Peak fractions were neutralized with 2.5 M Tris pH 7.2 and pooled. The neutralized fraction pools were either dialyzed into 1×dPBS for assays and biophysical characterization or utilized for bispecific DuoBody assembly. The protein concentration for each elution pool was determined by measuring absorbance at OD280 nm and calculated using absorbance extinction coefficient based on the amino acid sequence.

Sequences of representative PSMA antibodies are provided in Tables 4-12. Table 4 provides VH amino acid sequences, VL sequences, heavy chain sequences, and light chain sequences of various PSMA antibodies. VH CDR and VL CDR sequences are provided in Table 5 (Kabat), Table 6 (Chothia), Table 7 (AbM), Table 8 (Contact), and Table 9 (IMGT). Numbers beneath each sequence in the Tables denote corresponding SEQ ID NOs.

C-terminal Lysine 477 (CTL) can be added to the antibody heavy chain for optimized expression. The anti-PSMA antibodies PSMB895, PSMB896, PSMB897, and PSMB898 are identical to PSMB946, PSMB947, PSMB948, and PSMB949, respectively with the exception of the addition of a C-terminal lysine (CTL) in the antibody heavy chain (K477).

That is, PSMB895 and PSMB946 have the same VH amino acid sequences, the same VL amino acid sequences, the same VH CDR1-3 amino acid sequences, and the same VL CDR1-3 amino acid sequences. PSMB895 and PSMB946 also have the same light chain amino acid sequences, but their heavy chain amino acid sequences differ in that the heavy chain amino acid sequence of PSMB946 further comprises an additional Lys (K) amino acid residue at the C-terminus of the PSMB895 heavy chain amino acid sequence. PSMB896 and PSMB947 have the same VH amino acid sequences, the same VL amino acid sequences, the same VH CDR1-3 amino acid sequences, and the same VL CDR1-3 amino acid sequences. PSMB896 and PSMB947 also have the same light chain amino acid sequences, but their heavy chain amino acid sequences differ in that the heavy chain amino acid sequence of PSMB947 further comprises an additional Lys (K) amino acid residue at the C-terminus of the PSMB896 heavy chain amino acid sequence. PSMB897 and PSMB948 have the same VH amino acid sequences, the same VL amino acid sequences, the same VH CDR1-3 amino acid sequences, and the same VL CDR1-3 amino acid sequences. PSMB897 and PSMB948 also have the same light chain amino acid sequences, but their heavy chain amino acid sequences differ in that the heavy chain amino acid sequence of PSMB948 further comprises an additional Lys (K) amino acid residue at the C-terminus of the PSMB897 heavy chain amino acid sequence. PSMB898 and PSMB949 have the same VH amino acid sequences, the same VL amino acid sequences, the same VH CDR1-3 amino acid sequences, and the same VL CDR1-3 amino acid sequences. PSMB898 and PSMB949 also have the same light chain amino acid sequences, but their heavy chain amino acid sequences differ in that the heavy chain amino acid sequence of PSMB949 further comprises an additional Lys (K) amino acid residue at the C-terminus of the PSMB898 heavy chain amino acid sequence. The presence of the C-terminal Lysine can improve expression and manufacturing. C-terminal lysine (CTL) can be removed from the Ig constant region by endogenous circulating carboxypeptidases in the blood stream (Cai et al., (2011) Biotechnol Bioeng 108:404-412). During manufacturing, CTL removal can be controlled to less than the maximum level by control of concentration of extracellular Zn²⁺, EDTA or EDTA-Fe³⁺ as described in U.S. Patent Publ. No. US20140273092.

TABLE 4
PSMA Antibody VH, VL, HC and LC Amino Acid Sequences

| | Protein | | | | | | Light Chain |
|---|---|---|---|---|---|---|---|
| | Name | | | VH AA | VL AA | Heavy Chain | AA |
| # | (Target) | VH Name | VL Name | sequence | sequence | AA sequence | sequence |
| 1 | PSMB889 | PBD000101312 | PBD000101313 | EVQLVESGGG | QSVLTQPPSV | EVQLVESGGG | QSVLTQPPSV |
| | (PSMA) | | | LVKPGGSLRL | SGAPGQRVTI | LVKPGGSLRL | SGAPGQRVTI |
| | | | | SCAASGFTES | SCTGSSFNLG | SCAASGFTFS | SCTGSSFNLG |
| | | | | RYNMNWVRQA | AGYDVHWYQQ | RYNMNWVRQA | AGYDVHWYQQ |
| | | | | PGKGLEWVSS | VPGTVPKLLI | PGKGLEWVSS | VPGTVPKLLI |
| | | | | INSNSRYIYY | YDNSNRPSGV | INSNSRYIYY | YDNSNRPSGV |
| | | | | ADSVKGRFTI | PDRFSGSKSG | ADSVKGRFTI | PDRESGSKSG |
| | | | | SRDSAKNSLY | TSASLAITGL | SRDSAKNSLY | TSASLAITGL |
| | | | | LQMNSLRAED | QAEDETVYYC | LQMNSLRAED | QAEDETVYYC |
| | | | | TAVYYCAKTM | QSYDSSLSGV | TAVYYCAKTM | QSYDSSLSGV |
| | | | | GDYYYYYGMD | VFGGGTKLTV | GDYYYYYGMD | VFGGGTKLTV |
| | | | | VWGQGTTVTV | L | VWGQGTTVTV | LGQPKAAPSV |
| | | | | SS | | SSASTKGPSV | TLFPPSSEEL |
| | | | | | | FPLAPSSKST | QANKATLVCL |
| | | | | | | SGGTAALGCL | ISDFYPGAVT |
| | | | | | | VKDYFPEPVT | VAWKADSSPV |
| | | | | | | VSWNSGALTS | KAGVETTTPS |
| | | | | | | GVHTFPAVLQ | KQSNNKYAAS |
| | | | | | | SSGLYSLSSV | SYLSLTPEQW |
| | | | | | | VTVPSSSLGT | KSHRSYSCQV |
| | | | | | | QTYICNVNHK | THEGSTVEKT |
| | | | | | | PSNTKVDKKV | VAPTECS |
| | | | | | | EPKSCDKTHT | |
| | | | | | | CPPCPAPEAA | |
| | | | | | | GGPSVFLFPP | |
| | | | | | | KPKDTLMISR | |
| | | | | | | TPEVTCVVVS | |
| | | | | | | VSHEDPEVKF | |
| | | | | | | NWYVDGVEVH | |
| | | | | | | NAKTKPREEQ | |
| | | | | | | YNSTYRVVSV | |
| | | | | | | LTVLHQDWLN | |
| | | | | | | GKEYKCKVSN | |
| | | | | | | KALPAPIEKT | |
| | | | | | | ISKAKGQPRE | |
| | | | | | | PQVYTLPPSR | |
| | | | | | | EEMTKNQVSL | |
| | | | | | | TCLVKGFYPS | |
| | | | | | | DIAVEWESNG | |
| | | | | | | QPENNYKTTP | |
| | | | | | | PVLDSDGSFF | |
| | | | | | | LYSRLTVDKS | |
| | | | | | | RWQQGNVFSC | |
| | | | | | | SVMHEALHNH | |
| | | | | | | YTQKSLSLSP | |
| | | | | | | G | |
| | | | | 31 | 32 | 33 | 34 |
| 2 | PSMB890 | PBD000101312 | PBD000101314 | EVQLVESGGG | SSELTQPPSV | EVQLVESGGG | SSELTQPPSV |
| | (PSMA) | | | LVKPGGSLRL | SGAPGQRVTI | LVKPGGSLRL | SGAPGQRVTI |
| | | | | SCAASGFTFS | SCAGSLSNIG | SCAASGFTES | SCAGSLSNIG |
| | | | | RYNMNWVRQA | AGYDVHWYQQ | RYNMNWVRQA | AGYDVHWYQQ |
| | | | | PGKGLEWVSS | LPGTAPKLLI | PGKGLEWVSS | LPGTAPKLLI |
| | | | | INSNSRYIYY | YGNINRLSGV | INSNSRYIYY | YGNINRLSGV |
| | | | | ADSVKGRFTI | PERFSGSKSG | ADSVKGRFTI | PERFSGSKSG |
| | | | | SRDSAKNSLY | TSASLAITGL | SRDSAKNSLY | TSASLAITGL |
| | | | | LQMNSLRAED | QAEDGADYYC | LQMNSLRAED | QAEDGADYYC |
| | | | | TAVYYCAKTM | QSYDSSLSSY | TAVYYCAKTM | QSYDSSLSSY |
| | | | | GDYYYYYGMD | VFGTGTKVTV | GDYYYYYGMD | VFGTGTKVTV |
| | | | | VWGQGTTVTV | L | VWGQGTTVTV | LGQPKAAPSV |
| | | | | SS | | SSASTKGPSV | TLFPPSSEEL |
| | | | | | | FPLAPSSKST | QANKATLVCL |
| | | | | | | SGGTAALGCL | ISDFYPGAVT |
| | | | | | | VKDYFPEPVT | VAWKADSSPV |
| | | | | | | VSWNSGALTS | KAGVETTTPS |
| | | | | | | GVHTFPAVLQ | KQSNNKYAAS |
| | | | | | | SSGLYSLSSV | SYLSLTPEQW |
| | | | | | | VTVPSSSLGT | KSHRSYSCQV |
| | | | | | | QTYICNVNHK | THEGSTVEKT |
| | | | | | | PSNTKVDKKV | VAPTECS |
| | | | | | | EPKSCDKTHT | |
| | | | | | | CPPCPAPEAA | |
| | | | | | | GGPSVFLFPP | |
| | | | | | | KPKDTLMISR | |
| | | | | | | TPEVTCVVVS | |
| | | | | | | VSHEDPEVKF | |
| | | | | | | NWYVDGVEVH | |
| | | | | | | NAKTKPREEQ | |
| | | | | | | YNSTYRVVSV | |
| | | | | | | LTVLHQDWLN | |
| | | | | | | GKEYKCKVSN | |
| | | | | | | KALPAPIEKT | |
| | | | | | | ISKAKGQPRE | |
| | | | | | | PQVYTLPPSR | |
| | | | | | | EEMTKNQVSL | |
| | | | | | | TCLVKGFYPS | |
| | | | | | | DIAVEWESNG | |
| | | | | | | QPENNYKTTP | |
| | | | | | | PVLDSDGSFF | |
| | | | | | | LYSRLTVDKS | |
| | | | | | | RWQQGNVFSC | |
| | | | | | | SVMHEALHNH | |
| | | | | | | YTQKSLSLSP | |
| | | | | | | G | |
| | | | | 31 | 66 | 33 | 68 |
| 3 | PSMB891 | PBD000101316 | PBD000101315 | EVQLVESGGG | QSVLTQPPSA | EVQLVESGGG | QSVLTQPPSA |
| | (PSMA) | | | VVQPGRSLRL | SGTPGQGVTI | VVQPGRSLRL | SGTPGQGVTI |
| | | | | SCAASGFTFI | SCSGSSSNIG | SCAASGFTFI | SCSGSSSNIG |
| | | | | TYGMHWVRQA | SNTVNWFQQL | TYGMHWVRQA | SNTVNWFQQL |
| | | | | PGKGLEWVAV | PGTAPKLLIY | PGKGLEWVAV | PGTAPKLLIY |
| | | | | VSFDESNKYY | SDNQRPSGVP | VSFDESNKYY | SDNQRPSGVP |
| | | | | ADSVKGRFTI | DRESGSKSGT | ADSVKGRFTI | DRESGSKSGT |
| | | | | SRDNSKNTLY | SASLAISGLQ | SRDNSKNTLY | SASLAISGLQ |
| | | | | LQMNSLRAED | SEDEADYYCA | LQMNSLRAED | SEDEADYYCA |
| | | | | TAVYYCARAL | AWDDSLNGYV | TAVYYCARAL | AWDDSLNGYV |
| | | | | RDGNNWDYEN | FGTGTKVTVL | RDGNNWDYFN | FGTGTKVTVL |
| | | | | GMDVWGQGTT | | GMDVWGQGTT | GQPKAAPSVT |
| | | | | VTVSS | | VTVSSASTKG | LFPPSSEELQ |
| | | | | | | PSVFPLAPSS | ANKATLVCLI |
| | | | | | | KSTSGGTAAL | SDFYPGAVTV |
| | | | | | | GCLVKDYFPE | AWKADSSPVK |
| | | | | | | PVTVSWNSGA | AGVETTTPSK |
| | | | | | | LTSGVHTFPA | QSNNKYAASS |
| | | | | | | VLQSSGLYSL | YLSLTPEQWK |
| | | | | | | SSVVTVPSSS | SHRSYSCQVT |
| | | | | | | LGTQTYICNV | HEGSTVEKTV |
| | | | | | | NHKPSNTKVD | APTECS |
| | | | | | | KKVEPKSCDK | |
| | | | | | | THTCPPCPAP | |
| | | | | | | EAAGGPSVFL | |
| | | | | | | FPPKPKDTLM | |
| | | | | | | ISRTPEVTCV | |
| | | | | | | VVSVSHEDPE | |
| | | | | | | VKENWYVDGV | |
| | | | | | | EVHNAKTKPR | |
| | | | | | | EEQYNSTYRV | |
| | | | | | | VSVLTVLHQD | |
| | | | | | | WLNGKEYKCK | |
| | | | | | | VSNKALPAPI | |
| | | | | | | EKTISKAKGQ | |
| | | | | | | PREPQVYTLP | |
| | | | | | | PSREEMTKNQ | |
| | | | | | | VSLTCLVKGF | |
| | | | | | | YPSDIAVEWE | |
| | | | | | | SNGQPENNYK | |
| | | | | | | TTPPVLDSDG | |
| | | | | | | SFFLYSRLTV | |
| | | | | | | DKSRWQQGNV | |
| | | | | | | FSCSVMHEAL | |
| | | | | | | HNHYTQKSLS | |
| | | | | | | LSPG | |
| | | | | 99 | 100 | 101 | 102 |
| 4 | PSMB892 | PBD000101316 | PBD000101317 | EVQLVESGGG | QSVLTQPPSV | EVQLVESGGG | QSVLTQPPSV |
| | (PSMA) | | | VVQPGRSLRL | SGAPGQRVTI | VVQPGRSLRL | SGAPGQRVTI |
| | | | | SCAASGFTFI | SCTGSSSNIG | SCAASGFTFI | SCTGSSSNIG |
| | | | | TYGMHWVRQA | ADYDVHWYQH | TYGMHWVRQA | ADYDVHWYQH |
| | | | | PGKGLEWVAV | LPGTAPKLLI | PGKGLEWVAV | LPGTAPKLLI |
| | | | | VSEDESNKYY | YGNSNRPSGV | VSFDESNKYY | YGNSNRPSGV |
| | | | | ADSVKGRFTI | PDRFSGSKSG | ADSVKGRFTI | PDRESGSKSG |
| | | | | SRDNSKNTLY | TSASLAITGL | SRDNSKNTLY | TSASLAITGL |
| | | | | LQMNSLRAED | QAEDETDYYC | LQMNSLRAED | QAEDETDYYC |
| | | | | TAVYYCARAL | QSYDSSLSGW | TAVYYCARAL | QSYDSSLSGW |
| | | | | RDGNNWDYEN | VFGGGTKLTV | RDGNNWDYFN | VFGGGTKLTV |
| | | | | GMDVWGQGTT | L | GMDVWGQGTT | LGQPKAAPSV |
| | | | | VTVSS | | VTVSSASTKG | TLFPPSSEEL |
| | | | | | | PSVFPLAPSS | QANKATLVCL |
| | | | | | | KSTSGGTAAL | ISDFYPGAVT |
| | | | | | | GCLVKDYFPE | VAWKADSSPV |
| | | | | | | PVTVSWNSGA | KAGVETTTPS |
| | | | | | | LTSGVHTFPA | KQSNNKYAAS |
| | | | | | | VLQSSGLYSL | SYLSLTPEQW |
| | | | | | | SSVVTVPSSS | KSHRSYSCQV |
| | | | | | | LGTQTYICNV | THEGSTVEKT |
| | | | | | | NHKPSNTKVD | VAPTECS |
| | | | | | | KKVEPKSCDK | |
| | | | | | | THTCPPCPAP | |
| | | | | | | EAAGGPSVFL | |
| | | | | | | FPPKPKDTLM | |
| | | | | | | ISRTPEVTCV | |
| | | | | | | VVSVSHEDPE | |
| | | | | | | VKFNWYVDGV | |
| | | | | | | EVHNAKTKPR | |
| | | | | | | EEQYNSTYRV | |
| | | | | | | VSVLTVLHQD | |
| | | | | | | WLNGKEYKCK | |
| | | | | | | VSNKALPAPI | |
| | | | | | | EKTISKAKGQ | |
| | | | | | | PREPQVYTLP | |
| | | | | | | PSREEMTKNQ | |
| | | | | | | VSLTCLVKGF | |
| | | | | | | YPSDIAVEWE | |
| | | | | | | SNGQPENNYK | |
| | | | | | | TTPPVLDSDG | |
| | | | | | | SFFLYSRLTV | |
| | | | | | | DKSRWQQGNV | |
| | | | | | | FSCSVMHEAL | |
| | | | | | | HNHYTQKSLS | |
| | | | | | | LSPG | |
| | | | | 99 | 134 | 101 | 136 |
| 5 | PSMB893 | PBD000101318 | PBD000101315 | QVQLVESGGG | QSVLTQPPSA | QVQLVESGGG | QSVLTQPPSA |
| | (PSMA) | | | VVQPGRSLRL | SGTPGQGVTI | VVQPGRSLRL | SGTPGQGVTI |
| | | | | SCVASGFTFS | SCSGSSSNIG | SCVASGFTES | SCSGSSSNIG |
| | | | | SYGIHWVRQA | SNTVNWFQQL | SYGIHWVRQA | SNTVNWFQQL |
| | | | | PGKGLEWVAV | PGTAPKLLIY | PGKGLEWVAV | PGTAPKLLIY |
| | | | | IWYDGSNKYY | SDNQRPSGVP | IWYDGSNKYY | SDNQRPSGVP |
| | | | | ADSVKGRFTI | DRFSGSKSGT | ADSVKGRFTI | DRESGSKSGT |
| | | | | SRDNSKNTLY | SASLAISGLQ | SRDNSKNTLY | SASLAISGLQ |
| | | | | LQMNSLRAED | SEDEADYYCA | LQMNSLRAED | SEDEADYYCA |
| | | | | TAVYYSVRGV | AWDDSLNGYV | TAVYYSVRGV | AWDDSLNGYV |
| | | | | GPTSYYYNYG | FGTGTKVTVL | GPTSYYYNYG | FGTGTKVTVL |
| | | | | MDVWGQGTTV | | MDVWGQGTTV | GQPKAAPSVT |
| | | | | TVSS | | TVSSASTKGP | LFPPSSEELQ |
| | | | | | | SVFPLAPSSK | ANKATLVCLI |
| | | | | | | STSGGTAALG | SDFYPGAVTV |
| | | | | | | CLVKDYFPEP | AWKADSSPVK |
| | | | | | | VTVSWNSGAL | AGVETTTPSK |
| | | | | | | TSGVHTFPAV | QSNNKYAASS |
| | | | | | | LQSSGLYSLS | YLSLTPEQWK |
| | | | | | | SVVTVPSSSL | SHRSYSCQVT |
| | | | | | | GTQTYICNVN | HEGSTVEKTV |
| | | | | | | HKPSNTKVDK | APTECS |
| | | | | | | KVEPKSCDKT | |
| | | | | | | HTCPPCPAPE | |
| | | | | | | AAGGPSVFLF | |
| | | | | | | PPKPKDTLMI | |
| | | | | | | SRTPEVTCVV | |
| | | | | | | VSVSHEDPEV | |
| | | | | | | KFNWYVDGVE | |
| | | | | | | VHNAKTKPRE | |
| | | | | | | EQYNSTYRVV | |
| | | | | | | SVLTVLHQDW | |
| | | | | | | LNGKEYKCKV | |
| | | | | | | SNKALPAPIE | |
| | | | | | | KTISKAKGQP | |
| | | | | | | REPQVYTLPP | |
| | | | | | | SREEMTKNQV | |
| | | | | | | SLTCLVKGFY | |
| | | | | | | PSDIAVEWES | |
| | | | | | | NGQPENNYKT | |
| | | | | | | TPPVLDSDGS | |
| | | | | | | FFLYSRLTVD | |
| | | | | | | KSRWQQGNVF | |
| | | | | | | SCSVMHEALH | |
| | | | | | | NHYTQKSLSL | |
| | | | | | | SPG | |
| | | | | 167 | 100 | 169 | 102 |
| 6 | PSMB894 | PBD000101318 | PBD000101317 | QVQLVESGGG | QSVLTQPPSV | QVQLVESGGG | QSVLTQPPSV |
| | (PSMA) | | | VVQPGRSLRL | SGAPGQRVTI | VVQPGRSLRL | SGAPGQRVTI |
| | | | | SCVASGFTFS | SCTGSSSNIG | SCVASGFTES | SCTGSSSNIG |
| | | | | SYGIHWVRQA | ADYDVHWYQH | SYGIHWVRQA | ADYDVHWYQH |
| | | | | PGKGLEWVAV | LPGTAPKLLI | PGKGLEWVAV | LPGTAPKLLI |
| | | | | IWYDGSNKYY | YGNSNRPSGV | IWYDGSNKYY | YGNSNRPSGV |
| | | | | ADSVKGRFTI | PDRFSGSKSG | ADSVKGRFTI | PDRESGSKSG |
| | | | | SRDNSKNTLY | TSASLAITGL | SRDNSKNTLY | TSASLAITGL |
| | | | | LQMNSLRAED | QAEDETDYYC | LQMNSLRAED | QAEDETDYYC |
| | | | | TAVYYSVRGV | QSYDSSLSGW | TAVYYSVRGV | QSYDSSLSGW |
| | | | | GPTSYYYNYG | VFGGGTKLTV | GPTSYYYNYG | VFGGGTKLTV |
| | | | | MDVWGQGTTV | L | MDVWGQGTTV | LGQPKAAPSV |
| | | | | TVSS | | TVSSASTKGP | TLFPPSSEEL |
| | | | | | | SVFPLAPSSK | QANKATLVCL |
| | | | | | | STSGGTAALG | ISDFYPGAVT |
| | | | | | | CLVKDYFPEP | VAWKADSSPV |
| | | | | | | VTVSWNSGAL | KAGVETTTPS |
| | | | | | | TSGVHTFPAV | KQSNNKYAAS |
| | | | | | | LQSSGLYSLS | SYLSLTPEQW |
| | | | | | | SVVTVPSSSL | KSHRSYSCQV |
| | | | | | | GTQTYICNVN | THEGSTVEKT |
| | | | | | | HKPSNTKVDK | VAPTECS |
| | | | | | | KVEPKSCDKT | |
| | | | | | | HTCPPCPAPE | |
| | | | | | | AAGGPSVFLF | |
| | | | | | | PPKPKDTLMI | |
| | | | | | | SRTPEVTCVV | |
| | | | | | | VSVSHEDPEV | |
| | | | | | | KFNWYVDGVE | |
| | | | | | | VHNAKTKPRE | |
| | | | | | | EQYNSTYRVV | |
| | | | | | | SVLTVLHQDW | |
| | | | | | | LNGKEYKCKV | |
| | | | | | | SNKALPAPIE | |
| | | | | | | KTISKAKGQP | |
| | | | | | | REPQVYTLPP | |
| | | | | | | SREEMTKNQV | |
| | | | | | | SLTCLVKGFY | |
| | | | | | | PSDIAVEWES | |
| | | | | | | NGQPENNYKT | |
| | | | | | | TPPVLDSDGS | |
| | | | | | | FFLYSRLTVD | |
| | | | | | | KSRWQQGNVF | |
| | | | | | | SCSVMHEALH | |
| | | | | | | NHYTQKSLSL | |
| | | | | | | SPG | |
| | | | | 167 | 134 | 169 | 136 |
| 7 | PSMB895 | PBD000101320 | PBD000101319 | EVQLVESGGG | QSVLTQPPSV | EVQLVESGGG | QSVLTQPPSV |
| | (PSMA) | | | LVQPGGSLRL | SAAPGQKVTI | LVQPGGSLRL | SAAPGQKVTI |
| | | | | SCAASGFTFS | SCSGSSSNIG | SCAASGFTFS | SCSGSSSNIG |
| | | | | SYAMSWVRQA | NNYVSWYQQL | SYAMSWVRQA | NNYVSWYQQL |
| | | | | PGKGLEWVSA | PGTAPKLLIY | PGKGLEWVSA | PGTAPKLLIY |
| | | | | ISGGIGSTYY | DNNKRPSGIP | ISGGIGSTYY | DNNKRPSGIP |
| | | | | ADSVKGRFTI | DRFSGSKSGT | ADSVKGRFTI | DRESGSKSGT |
| | | | | SRDNSKNTLW | SATLGITGLQ | SRDNSKNTLW | SATLGITGLQ |
| | | | | LQMNSLRAED | TGDEADYYCG | LQMNSLRAED | TGDEADYYCG |
| | | | | TAVYYCAKD | TWDSSLSAYV | TAVYYCAKDG | TWDSSLSAYV |
| | | | | GVGATPYYED | FGTGTKVTVL | VGATPYYFDY | FGTGTKVTVL |
| | | | | YWGQGTLVTV | | WGQGTLVTVS | GQPKAAPSVT |
| | | | | SS | | SASTKGPSVF | LFPPSSEELQ |
| | | | | | | PLAPSSKSTS | ANKATLVCLI |
| | | | | | | GGTAALGCLV | SDFYPGAVTV |
| | | | | | | KDYFPEPVTV | AWK |
| | | | | | | SWNSGALTSG | ADSSPVKAGV |
| | | | | | | VHTFPAVLQS | ETTTPSKQSN |
| | | | | | | SGLYSLSSVV | NKYAASSYLS |
| | | | | | | TVPSSSLGTQ | LTPEQWKSHR |
| | | | | | | TYICNVNHKP | SYSCQVTHEG |
| | | | | | | SNTKVDKKVE | STVEKTVAPT |
| | | | | | | PKSCDKTHTC | ECS |
| | | | | | | PPCPAPEAAG | |
| | | | | | | GPSVFLFPPK | |
| | | | | | | PKDTLMISRT | |
| | | | | | | PEVTCVVVSV | |
| | | | | | | SHEDPEVKFN | |
| | | | | | | WYVDGVEVHN | |
| | | | | | | AKTKPREEQY | |
| | | | | | | NSTYRVVSVL | |
| | | | | | | TVLHQDWLNG | |
| | | | | | | KEYKCKVSNK | |
| | | | | | | ALPAPIEKTI | |
| | | | | | | SKAKGQPREP | |
| | | | | | | QVYTLPPSRE | |
| | | | | | | EMTKNQVSLT | |
| | | | | | | CLVKGFYPSD | |
| | | | | | | IAVEWESNGQ | |
| | | | | | | PENNYKTTPP | |
| | | | | | | VLDSDGSFFL | |
| | | | | | | YSRLTVDKSR | |
| | | | | | | WQQGNVFSCS | |
| | | | | | | VMHEALHNHY | |
| | | | | | | TQKSLSLSPG | |
| | | | | 235 | 236 | 237 | 238 |
| 8 | PSMB896 | PBD000101320 | PBD000101321 | EVQLVESGGG | QSVLTQPPSV | EVQLVESGGG | QSVLTQPPSV |
| | (PSMA) | | | LVQPGGSLRL | SAAPGQKVTI | LVQPGGSLRL | SAAPGQKVTI |
| | | | | SCAASGFTES | SCSGSSSNIG | SCAASGFTES | SCSGSSSNIG |
| | | | | SYAMSWVRQA | INYVSWYQQL | SYAMSWVRQA | INYVSWYQQL |
| | | | | PGKGLEWVSA | PGTAPKLLIY | PGKGLEWVSA | PGTAPKLLIY |
| | | | | ISGGIGSTYY | DNNKRPSGIP | ISGGIGSTYY | DNNKRPSGIP |
| | | | | ADSVKGRFTI | DRFSGSKSGT | ADSVKGRFTI | DRESGSKSGT |
| | | | | SRDNSKNTLW | SATLGITGLQ | SRDNSKNTLW | SATLGITGLQ |
| | | | | LQMNSLRAED | TGDEADYYCG | LQMNSLRAED | TGDEADYYCG |
| | | | | TAVYYCAKDG | TWDSSLSAVV | TAVYYCAKDG | TWDSSLSAVV |
| | | | | VGATPYYFDY | FGGGTKLTVL | VGATPYYFDY | FGGGTKLTVL |
| | | | | WGQGTLVTVS | | WGQGTLVTVS | GQPKAAPSVT |
| | | | | S | | SASTKGPSVF | LFPPSSEELQ |
| | | | | | | PLAPSSKSTS | ANKATLVCLI |
| | | | | | | GGTAALGCLV | SDFYPGAVTV |
| | | | | | | KDYFPEPVTV | AWKADSSPVK |
| | | | | | | SWNSGALTSG | AGVETTTPSK |
| | | | | | | VHTFPAVLQS | QSNNKYAASS |
| | | | | | | SGLYSLSSVV | YLSLTPEQWK |
| | | | | | | TVPSSSLGTQ | SHRSYSCQVT |
| | | | | | | TYICNVNHKP | HEGSTVEKTV |
| | | | | | | SNTKVDKKVE | APTECS |
| | | | | | | PKSCDKTHTC | |
| | | | | | | PPCPAPEAAG | |
| | | | | | | GPSVFLFPPK | |
| | | | | | | PKDTLMISRT | |
| | | | | | | PEVTCVVVSV | |
| | | | | | | SHEDPEVKFN | |
| | | | | | | WYVDGVEVHN | |
| | | | | | | AKTKPREEQY | |
| | | | | | | NSTYRVVSVL | |
| | | | | | | TVLHQDWLNG | |
| | | | | | | KEYKCKVSNK | |
| | | | | | | ALPAPIEKTI | |
| | | | | | | SKAKGQPREP | |
| | | | | | | QVYTLPPSRE | |
| | | | | | | EMTKNQVSLT | |
| | | | | | | CLVKGFYPSD | |
| | | | | | | IAVEWESNGQ | |
| | | | | | | PENNYKTTPP | |
| | | | | | | VLDSDGSFFL | |
| | | | | | | YSRLTVDKSR | |
| | | | | | | WQQGNVFSCS | |
| | | | | | | VMHEALHNHY | |
| | | | | | | TQKSLSLSPG | |
| | | | | 235 | 270 | 237 | 272 |
| 9 | PSMB897 | PBD000101322 | PBD000101319 | EVQLVESGGG | QSVLTQPPSV | EVQLVESGGG | QSVLTQPPSV |
| | (PSMA) | | | LVQPGGSLRL | SAAPGQKVTI | LVQPGGSLRL | SAAPGQKVTI |
| | | | | SCAASGFTES | SCSGSSSNIG | SCAASGFTFS | SCSGSSSNIG |
| | | | | SYAMSWVRQA | NNYVSWYQQL | SYAMSWVRQA | NNYVSWYQQL |
| | | | | PGKGLEWVSA | PGTAPKLLIY | PGKGLEWVSA | PGTAPKLLIY |
| | | | | ISGGSGSTYY | DNNKRPSGIP | ISGGSGSTYY | DNNKRPSGIP |
| | | | | ADSVKGRFTI | DRESGSKSGT | ADSVKGRFTI | DRESGSKSGT |
| | | | | SRDNSKNTLY | SATLGITGLQ | SRDNSKNTLY | SATLGITGLQ |
| | | | | LQMNSLRAED | TGDEADYYCG | LQMNSLRAED | TGDEADYYCG |
| | | | | SAVYYCAKDG | TWDSSLSAYV | SAVYYCAKDG | TWDSSLSAYV |
| | | | | VGATPYYFDY | FGTGTKVTVL | VGATPYYFDY | FGTGTKVTVL |
| | | | | WGQGTLVTVS | | WGQGTLVTVS | GQPKAAPSVT |
| | | | | S | | SASTKGPSVF | LFPPSSEELQ |
| | | | | | | PLAPSSKSTS | ANKATLVCLI |
| | | | | | | GGTAALGCLV | SDFYPGAVTV |
| | | | | | | KDYFPEPVTV | AWKADSSPVK |
| | | | | | | SWNSGALTSG | AGVETTTPSK |
| | | | | | | VHTFPAVLQS | QSNNKYAASS |
| | | | | | | SGLYSLSSVV | YLSLTPEQWK |
| | | | | | | TVPSSSLGTQ | SHRSYSCQVT |
| | | | | | | TYICNVNHKP | HEGSTVEKTV |
| | | | | | | SNTKVDKKVE | APTECS |
| | | | | | | PKSCDKTHTC | |
| | | | | | | PPCPAPEAAG | |
| | | | | | | GPSVFLFPPK | |
| | | | | | | PKDTLMISRT | |
| | | | | | | PEVTCVVVSV | |
| | | | | | | SHEDPEVKEN | |
| | | | | | | WYVDGVEVHN | |
| | | | | | | AKTKPREEQY | |
| | | | | | | NSTYRVVSVL | |
| | | | | | | TVLHQDWLNG | |
| | | | | | | KEYKCKVSNK | |
| | | | | | | ALPAPIEKTI | |
| | | | | | | SKAKGQPREP | |
| | | | | | | QVYTLPPSRE | |
| | | | | | | EMTKNQVSLT | |
| | | | | | | CLVKGFYPSD | |
| | | | | | | IAVEWESNGQ | |
| | | | | | | PENNYKTTPP | |
| | | | | | | VLDSDGSFFL | |
| | | | | | | YSRLTVDKSR | |
| | | | | | | WQQGNVFSCS | |
| | | | | | | VMHEALHNHY | |
| | | | | | | TQKSLSLSPG | |
| | | | | 303 | 236 | 305 | 238 |
| 10 | PSMB898 | PBD000101322 | PBD000101321 | EVQLVESGGG | QSVLTQPPSV | EVQLVESGGG | QSVLTQPPSV |
| | (PSMA) | | | LVQPGGSLRL | SAAPGQKVTI | LVQPGGSLRL | SAAPGQKVTI |
| | | | | SCAASGFTES | SCSGSSSNIG | SCAASGFTES | SCSGSSSNIG |
| | | | | SYAMSWVRQA | INYVSWYQQL | SYAMSWVRQA | INYVSWYQQL |
| | | | | PGKGLEWVSA | PGTAPKLLIY | PGKGLEWVSA | PGTAPKLLIY |
| | | | | ISGGSGSTYY | DNNKRPSGIP | ISGGSGSTYY | DNNKRPSGIP |
| | | | | ADSVKGRFTI | DRESGSKSGT | ADSVKGRFTI | DRESGSKSGT |
| | | | | SRDNSKNTLY | SATLGITGLQ | SRDNSKNTLY | SATLGITGLQ |
| | | | | LQMNSLRAED | TGDEADYYCG | LQMNSLRAED | TGDEADYYCG |
| | | | | SAVYYCAKDG | TWDSSLSAVV | SAVYYCAKDG | TWDSSLSAVV |
| | | | | VGATPYYFDY | FGGGTKLTVL | VGATPYYFDY | FGGGTKLTVL |
| | | | | WGQGTLVTVS | | WGQGTLVTVS | GQPKAAPSVT |
| | | | | S | | SASTKGPSVF | LFPPSSEELQ |
| | | | | | | PLAPSSKSTS | ANKATLVCLI |
| | | | | | | GGTAALGCLV | SDFYPGAVTV |
| | | | | | | KDYFPEPVTV | AWKADSSPVK |
| | | | | | | SWNSGALTSG | AGVETTTPSK |
| | | | | | | VHTFPAVLQS | QSNNKYAASS |
| | | | | | | SGLYSLSSVV | YLSLTPEQWK |
| | | | | | | TVPSSSLGTQ | SHRSYSCQVT |
| | | | | | | TYICNVNHKP | HEGSTVEKTV |
| | | | | | | SNTKVDKKVE | APTECS |
| | | | | | | PKSCDKTHTC | |
| | | | | | | PPCPAPEAAG | |
| | | | | | | GPSVFLFPPK | |
| | | | | | | PKDTLMISRT | |
| | | | | | | PEVTCVVVSV | |
| | | | | | | SHEDPEVKFN | |
| | | | | | | WYVDGVEVHN | |
| | | | | | | AKTKPREEQY | |
| | | | | | | NSTYRVVSVL | |
| | | | | | | TVLHQDWLNG | |
| | | | | | | KEYKCKVSNK | |
| | | | | | | ALPAPIEKTI | |
| | | | | | | SKAKGQPREP | |
| | | | | | | QVYTLPPSRE | |
| | | | | | | EMTKNQVSLT | |
| | | | | | | CLVKGFYPSD | |
| | | | | | | IAVEWESNGQ | |
| | | | | | | PENNYKTTPP | |
| | | | | | | VLDSDGSFFL | |
| | | | | | | YSRLTVDKSR | |
| | | | | | | WQQGNVFSCS | |
| | | | | | | VMHEALHNHY | |
| | | | | | | TQKSLSLSPG | |
| | | | | 303 | 270 | 305 | 272 |
| 11 | PSMB899 | PBD000101324 | PBD000101323 | EVQLVESGGG | QSVLTQPPSV | EVQLVESGGG | QSVLTQPPSV |
| | (PSMA) | | | LVQPGGSLRL | SAAPGQKVTI | LVQPGGSLRL | SAAPGQKVTI |
| | | | | SCTASGFIFS | SCSGSSSNIG | SCTASGFIES | SCSGSSSNIG |
| | | | | SYAMSWVRQA | NNYVSWYQQL | SYAMSWVRQA | NNYVSWYQQL |
| | | | | PGKGLEWVSA | PGTAPKLLIF | PGKGLEWVSA | PGTAPKLLIE |
| | | | | ISGGYGAPYY | DNNKRPSGIP | ISGGYGAPYY | DNNKRPSGIP |
| | | | | ADTVKGRFTI | DRESGSKSGT | ADTVKGRFTI | DRESGSKSGT |
| | | | | SRDNSKNTLY | SATLGITGLQ | SRDNSKNTLY | SATLGITGLQ |
| | | | | LQMNSLRAED | TGDEADYYCG | LQMNSLRAED | TGDEADYYCG |
| | | | | TAVYYCAKDG | TWDSSLSAYV | TAVYYCAKDG | TWDSSLSAYV |
| | | | | VGATPYYFDD | FGTGTKVTVL | VGATPYYFDD | FGTGTKVTVL |
| | | | | WGQGILVTVS | | WGQGILVTVS | GQPKAAPSVT |
| | | | | S | | SASTKGPSVF | LFPPSSEELQ |
| | | | | | | PLAPSSKSTS | ANKATLVCLI |
| | | | | | | GGTAALGCLV | SDFYPGAVTV |
| | | | | | | KDYFPEPVTV | AWKADSSPVK |
| | | | | | | SWNSGALTSG | AGVETTTPSK |
| | | | | | | VHTFPAVLQS | QSNNKYAASS |
| | | | | | | SGLYSLSSVV | YLSLTPEQWK |
| | | | | | | TVPSSSLGTQ | SHRSYSCQVT |
| | | | | | | TYICNVNHKP | HEGSTVEKTV |
| | | | | | | SNTKVDKKVE | APTECS |
| | | | | | | PKSCDKTHTC | |
| | | | | | | PPCPAPEAAG | |
| | | | | | | GPSVFLFPPK | |
| | | | | | | PKDTLMISRT | |
| | | | | | | PEVTCVVVSV | |
| | | | | | | SHEDPEVKFN | |
| | | | | | | WYVDGVEVHN | |
| | | | | | | AKTKPREEQY | |
| | | | | | | NSTYRVVSVL | |
| | | | | | | TVLHQDWLNG | |
| | | | | | | KEYKCKVSNK | |
| | | | | | | ALPAPIEKTI | |
| | | | | | | SKAKGQPREP | |
| | | | | | | QVYTLPPSRE | |
| | | | | | | EMTKNQVSLT | |
| | | | | | | CLVKGFYPSD | |
| | | | | | | IAVEWESNGQ | |
| | | | | | | PENNYKTTPP | |
| | | | | | | VLDSDGSFFL | |
| | | | | | | YSRLTVDKSR | |
| | | | | | | WQQGNVFSCS | |
| | | | | | | VMHEALHNHY | |
| | | | | | | TQKSLSLSPG | |
| | | | | 371 | 372 | 373 | 374 |
| 12 | PSMHB49 | NA | NA | EVQLVESGGG | EIVMTQSPGT | EVQLVESGGG | EIVMTQSPGT |
| | SC1133 | | | LVKPGGSLRL | LSLSPGERAT | LVKPGGSLRL | LSLSPGERAT |
| | 011A111 | | | SCVASGFTFS | LSCRASQSVS | SCVASGFTFS | LSCRASQSVS |
| | (PSMA) | | | FYSMNWVRQA | SSFLAWYQQK | FYSMNWVRQA | SSFLAWYQQK |
| | | | | PGKGLDWVSS | PGQAPRLLIS | PGKGLDWVSS | PGQAPRLLIS |
| | | | | ISSSGNYIYY | GASSRATGIP | ISSSGNYIYY | GASSRATGIP |
| | | | | ADSVKGRFTI | DRESVSGSGT | ADSVKGRFTI | DRFSVSGSGT |
| | | | | SRDNAKNSLH | DFTLTISRLE | SRDNAKNSLH | DFTLTISRLE |
| | | | | LHMNSLK | PEDFAVYYCQ | LHMNSLKAED | PEDFAVYYCQ |
| | | | | AEDTAMYFCA | QYGVSPWTFG | TAMYFCARSY | QYGVSPWTFG |
| | | | | RSYSGSYDAF | QGTKVEIK | SGSYDAFDFW | QGTKVEIKRT |
| | | | | DFWGQGTMVT | | GQGTMVTVSS | VAAPSVFIF |
| | | | | VSS | | ASTKGPSVFP | PPSDEQLKSG |
| | | | | | | LAPSSKSTSG | TASVVCLLNN |
| | | | | | | GTAALGCLVK | FYPREAKVQW |
| | | | | | | DYFPEPVTVS | KVDNALQSGN |
| | | | | | | WNSGALTSGV | SQESVTEQDS |
| | | | | | | HTFPAVLQSS | KDSTYSLSST |
| | | | | | | GLYSLSSVVT | LTLSKADYEK |
| | | | | | | VPSSSLGTQT | HKVYACEVTH |
| | | | | | | YICNVNHKPS | QGLSSPVTKS |
| | | | | | | NTKVDKKVEP | FNRGEC |
| | | | | | | KSCDKTHTCP | |
| | | | | | | PCPAPEAAGG | |
| | | | | | | PSVFLFPPKP | |
| | | | | | | KDTLMISRTP | |
| | | | | | | EVTCVVVSVS | |
| | | | | | | HEDPEVKFNW | |
| | | | | | | YVDGVEVHNA | |
| | | | | | | KTKPREEQYN | |
| | | | | | | STYRVVSVLT | |
| | | | | | | VLHQDWLNGK | |
| | | | | | | EYKCKVSNKA | |
| | | | | | | LPAPIEKTIS | |
| | | | | | | KAKGQPREPQ | |
| | | | | | | VYTLPPSREE | |
| | | | | | | MTKNQVSLSC | |
| | | | | | | AVKGFYPSDI | |
| | | | | | | AVEWESNGQP | |
| | | | | | | ENNYKTTPPV | |
| | | | | | | LDSDGSFFLV | |
| | | | | | | SKLTVDKSRW | |
| | | | | | | QQGNVFSCSV | |
| | | | | | | MHEALHNRFT | |
| | | | | | | QKSLSLSPGK | |
| | | | | 405 | 406 | 407 | 408 |
| 13 | PSMB896 | NA | NA | EVQLVESGGG | QSVLTQPPSV | EVQLVESGGG | QSVLTQPPSV |
| | -G100A | | | LVQPGGSLRL | SAAPGQKVTI | LVQPGGSLRL | SAAPGQKVTI |
| | (PSMA) | | | SCAASGFTES | SCSGSSSNIG | SCAASGFTES | SCSGSSSNIG |
| | | | | SYAMSWVRQA | INYVSWYQQL | SYAMSWVRQA | INYVSWYQQL |
| | | | | PGKGLEWVSA | PGTAPKLLIY | PGKGLEWVSA | PGTAPKLLIY |
| | | | | ISGGIGSTYY | DNNKRPSGIP | ISGGIGSTYY | DNNKRPSGIP |
| | | | | ADSVKGRFTI | DRESGSKSGT | ADSVKGRFTI | DRESGSKSGT |
| | | | | SRDNSKNTLW | SATLGITGLQ | SRDNSKNTLW | SATLGITGLQ |
| | | | | LQMNSLRAED | TGDEADYYCG | LQMNSLRAED | TGDEADYYCG |
| | | | | TAVYYCAKDA | TWDSSLSAVV | TAVYYCAKDA | TWDSSLSAVV |
| | | | | VGATPYYFDY | FGGGTKLTVL | VGATPYYFDY | FGGGTKLTVL |
| | | | | WGQGTLVTVS | | WGQGTLVTVS | GQPKAAPSVT |
| | | | | S | | SASTKGPSVF | LFPPSSEELQ |
| | | | | | | PLAPSSKSTS | ANKATLVCLI |
| | | | | | | GGTAALGCLV | SDFYPGAVTV |
| | | | | | | KDYFPEPVTV | AWKADSSPVK |
| | | | | | | SWNSGALTSG | AGVETTTPSK |
| | | | | | | VHTFPAVLQS | QSNNKYAASS |
| | | | | | | SGLYSLSSVV | YLSLTPEQWK |
| | | | | | | TVPSSSLGTQ | SHRSYSCQVT |
| | | | | | | TYICNVNHKP | HEGSTVEKTV |
| | | | | | | SNTKVDKKVE | APTECS |
| | | | | | | PKSCDKTHTC | |
| | | | | | | PPCPAPEAAG | |
| | | | | | | GPSVFLFPPK | |
| | | | | | | PKDTLMISRT | |
| | | | | | | PEVTCVVVSV | |
| | | | | | | SHEDPEVKFN | |
| | | | | | | WYVDGVEVHN | |
| | | | | | | AKTKPREEQY | |
| | | | | | | NSTYRVVSVL | |
| | | | | | | TVLHQDWLNG | |
| | | | | | | KEYKCKVSNK | |
| | | | | | | ALPAPIEKTI | |
| | | | | | | SKAKGQPREP | |
| | | | | | | QVYTLPPSRE | |
| | | | | | | EMTKNQVSLS | |
| | | | | | | CAVKGFYPSD | |
| | | | | | | IAVEWESNGQ | |
| | | | | | | PENNYKTTPP | |
| | | | | | | VLDSDGSFFL | |
| | | | | | | VSKLTVDKSR | |
| | | | | | | WQQGNVFSCS | |
| | | | | | | VMHEALHNRF | |
| | | | | | | TQKSLSLSPG | |
| | | | | | | K | |
| | | | | 439 | 270 | 441 | 272 |
| 14 | PSMA_P7 | NA | NA | QVQLVESGGG | SYELMQPPSV | NA | NA |
| | 2_A10- | | | VVQPGRSLRL | SVSPGQTARI | | |
| | HC-G54E | | | SCAASGFTFS | TCSGDALPKQ | | |
| | (PSMA) | | | SYNMNWVRQA | YAYWYQQKPG | | |
| | | | | PGKGLEWVAI | QAPVLVIYKD | | |
| | | | | IYYDESNKYY | SERPSGIPVR | | |
| | | | | ADSVKGRFTI | FSGSSSGTTV | | |
| | | | | SRDISKNTLY | TLTITGVQAE | | |
| | | | | LQMNSLRAED | DEADYYCQSA | | |
| | | | | TAVYYCARER | DSSGTYVFGT | | |
| | | | | GRDYYGMDVW | GTKVTVL | | |
| | | | | GQGTTVTVSS | | | |
| | | | | 473 | 474 | 475 | 476 |
| 15 | PSMA_P7 | NA | NA | EVQLVESGGD | QSVLTQPASV | NA | NA |
| | 2_D01- | | | LVQPGGSLRL | SGSPGQSITI | | |
| | HC-D95E | | | SCAASGFTEN | SCTGTSSDVG | | |
| | (PSMA) | | | NYNMNWVRQA | GYNYVSWYQQ | | |
| | | | | PGKGLEWVSH | HPGKAPKLMI | | |
| | | | | ISTSSSNKYY | YEVSNRPSGV | | |
| | | | | ADSVKGRESI | SNRFSGSKSG | | |
| | | | | SRDIAKNSMY | NTASLTISGL | | |
| | | | | LQMNSLRDED | QAEDEADYYC | | |
| | | | | TAVYYCAREG | SSYTSSYTYV | | |
| | | | | VGADYGDYYY | FGTGTKLTVL | | |
| | | | | YGMDVWGQGT | | | |
| | | | | TVTVSS | | | |
| | | | | 507 | 508 | 509 | 510 |
| 16 | PSMA_P7 | NA | NA | QVQLQESGGG | EIVLTQSPGT | NA | NA |
| | 2_F01 | | | VVQPGRSLRL | LSVSPGERAT | | |
| | and | | | SCAASGFTFS | LSCRASQSVR | | |
| | PSMA_P7 | | | TYGMHWVRQA | SNLAWYQQKP | | |
| | 5 F01 | | | PGKGLEWVAF | GQAPRLLIYG | | |
| | (PSMA) | | | ISYDGSNKYY | ASTRATGIPA | | |
| | | | | ADSVKGRFTI | RFSGSGSGTE | | |
| | | | | SRDNSKHTLY | FTLTISSLQS | | |
| | | | | LQMNSLRAED | EDFAVYYCHQ | | |
| | | | | TAVYYCAGRD | YNDWPPYTFG | | |
| | | | | NLRFLEWFMD | QGTKLEIK | | |
| | | | | VWGQGTTVTV | | | |
| | | | | SS | | | |
| | | | | 541 | 542 | 543 | 544 |
| 17 | PSMA_P7 | NA | NA | EVQLVESGGG | SYELTQPPSV | NA | NA |
| | 2_F07 | | | VVQPGRSLRL | SVAPGQTARI | | |
| | (PSMA) | | | SCAASGFTFS | TCGGNNIGSK | | |
| | | | | SYGMNWVRQA | SVHWYQQKPG | | |
| | | | | PGKGLEWVAV | QAPVLVVYDD | | |
| | | | | TSYDGSNKYY | SDRPSGIPER | | |
| | | | | ADSVKGRFTI | FSGSNSGNTA | | |
| | | | | SRDISKNTLY | TLTISRVEAG | | |
| | | | | LQMSSLRAED | DEADYYCQVW | | |
| | | | | TAVYYCARDP | DSSTDHVVFG | | |
| | | | | YSSSWNGAFD | GGTKLTVL | | |
| | | | | IWGPGTMVTV | | | |
| | | | | SS | | | |
| | | | | 575 | 576 | 577 | 578 |
| 18 | PSMA_P7 | NA | NA | EVQLVESGGG | QSVLTQPPSA | NA | NA |
| | 2_E07 | | | VVQPGRSLRL | SGTPGQGVTI | | |
| | (PSMA) | | | SCAASGFTFI | SCSGSSSNIG | | |
| | | | | TYGMHWVRQA | SNTVNWFQQL | | |
| | | | | PGKGLEWVAV | PGTAPKLLIY | | |
| | | | | VSFDESNKYY | SDNQRPSGVP | | |
| | | | | ADSVKGRFTI | DRESGSKSGT | | |
| | | | | SRDNSKNTLY | SASLAISGLQ | | |
| | | | | LQMNSLRAED | SEDEADYYCA | | |
| | | | | TAVYYCARAL | AWDDSLNGYV | | |
| | | | | RDGNNWDYFN | FGTGTKVTVL | | |
| | | | | GMDVWGQGTT | | | |
| | | | | VTVSS | | | |
| | | | | 99 | 100 | 611 | 612 |
| 19 | PSMA_P7 | NA | NA | EVQLVESGGD | QSVLTQPASV | NA | NA |
| | 2_D01 | | | LVQPGGSLRL | SGSPGQSITI | | |
| | (PSMA) | | | SCAASGFTEN | SCTGTSSDVG | | |
| | | | | NYNMNWVRQA | GYNYVSWYQQ | | |
| | | | | PGKGLEWVSH | HPGKAPKLMI | | |
| | | | | ISTSSSNKYY | YEVSNRPSGV | | |
| | | | | ADSVKGRFSI | SNRFSGSKSG | | |
| | | | | SRDIAKNSMY | NTASLTISGL | | |
| | | | | LQMNSLRDED | QAEDEADYYC | | |
| | | | | TAVYYCARDG | SSYTSSYTYV | | |
| | | | | VGADYGDYYY | FGTGTKLTVL | | |
| | | | | YGMDVWGQGT | | | |
| | | | | TVTVSS | | | |
| | | | | 643 | 508 | 645 | 646 |
| 20 | PSMA_P7 | NA | NA | QVQLVESGGG | QSVLTQPPSV | NA | NA |
| | 2_C01 | | | EVQPGRSLRL | SVAPGQTARI | | |
| | (PSMA) | | | SCAASGFSFS | TCGGNNSGSK | | |
| | | | | GYGMHWVRQA | SVHWYQQKPG | | |
| | | | | PGKGLEWVAV | QAPVLVVYDD | | |
| | | | | MSYDGSNRFY | SDRPSGIPER | | |
| | | | | VDSVRGRFSI | FSGSNSGNTA | | |
| | | | | SRDNSKNTLY | TLTISRVEAG | | |
| | | | | LQMNSLRPED | DEADYYCQVW | | |
| | | | | TAVYYCARDT | DSSSDHGVFG | | |
| | | | | VWGSHPDAFD | GGTKLTVL | | |
| | | | | IWGQGTVVTV | | | |
| | | | | SS | | | |
| | | | | 677 | 678 | 679 | 680 |
| 21 | PSMA_P7 | NA | NA | QVQLVESGGG | SYELMQPPSV | NA | NA |
| | 2_A10 | | | VVQPGRSLRL | SVSPGQTARI | | |
| | (PSMA) | | | SCAASGFTES | TCSGDALPKQ | | |
| | | | | SYNMNWVRQA | YAYWYQQKPG | | |
| | | | | PGKGLEWVAI | QAPVLVIYKD | | |
| | | | | IYYDGSNKYY | SERPSGIPVR | | |
| | | | | ADSVKGRFTI | FSGSSSGTTV | | |
| | | | | SRDISKNTLY | TLTITGVQAE | | |
| | | | | LQMNSLRAED | DEADYYCQSA | | |
| | | | | TAVYYCARER | DSSGTYVFGT | | |
| | | | | GRDYYGMDVW | GTKVTVL | | |
| | | | | GQGTTVTVSS | | | |
| | | | | 711 | 474 | 713 | 714 |
| 22 | PSMA_ | NA | NA | EVOLLESGPG | QSVLTQPPSA | NA | NA |
| | P72_ | | | LVKPSETLSL | SGTPGQRVTI | | |
| | F02 | | | TCTVSGGSII | SCSGSSSNIG | | |
| | and | | | SYYWSWIRQP | SNTVNWYQQL | | |
| | PSMA_ | | | AGKGLEWIGR | PGTAPKLLIY | | |
| | P70_ | | | IYSSGSTNYN | SSNQRPSGVP | | |
| | F02 | | | PSLKSRVTMS | DRFSGSKSGT | | |
| | (PSMA) | | | VDTSKNQFSL | SASLAISGLQ | | |
| | | | | KLSSVTAADT | SEDEADYYCA | | |
| | | | | AVYYCAKVGV | AWDDSLNGVV | | |
| | | | | WPGAFDIWGQ | FGGGTKLTVL | | |
| | | | | GTMVTVSS | | | |
| | | | | 745 | 746 | 747 | 748 |
| 23 | PSMA_ | NA | NA | EVOLVESGGG | QSALTQPASV | NA | NA |
| | P72_ | | | VVQPGRSLRL | SGSPGQSITI | | |
| | G02 | | | SCAASGFSFS | SCTGASSDVG | | |
| | (PSMA) | | | GYGMHWVRQA | GYNYVSWYQQ | | |
| | | | | PGKGLEWVAV | HPGKAPKLMI | | |
| | | | | ISYDGSNKYY | YEVSNRPSGV | | |
| | | | | ADSVKGRFTI | SNRFSGSKSG | | |
| | | | | SRDNSKNTLY | NTASLTISGL | | |
| | | | | LQMNSLRVED | QAEDEADYYC | | |
| | | | | TAVYYCARDR | SSYTITSTLV | | |
| | | | | IWGSRGYYYG | FGGGTKLTVL | | |
| | | | | MDVWGQGTTV | | | |
| | | | | TVSS | | | |
| | | | | 779 | 780 | 781 | 782 |
| 24 | PSMA_ | NA | NA | QVQLQESGGD | SYELTQPPSV | NA | NA |
| | P72_ | | | VVQPGRSLRL | SVAPGQTARI | | |
| | A11 | | | SC | TCGGNNIGSK | | |
| | (PSMA) | | | AASGFSFSGY | SVHWYQQKPG | | |
| | | | | GLHWVRQAPG | QAPVLVVYDD | | |
| | | | | RGLEWVTLIS | SDRPSGIPER | | |
| | | | | YDGSNKYYAD | FSGTNSGNTA | | |
| | | | | SVKGRFTISR | TLTISRAEAG | | |
| | | | | DNSKNTLYLQ | DEADYYCQVW | | |
| | | | | MNSLRAEDTA | DSSSDHVVFG | | |
| | | | | VYYCAKTTVS | GGTKLTVL | | |
| | | | | DPYYYGMDVW | | | |
| | | | | GQGTTVTVSS | | | |
| | | | | 813 | 814 | 815 | 816 |

TABLE 5
PSMA Antibody Kabat CDR Amino Acid Sequences

| | Protein | HC Kabat | HC Kabat | HC Kabat | LC Kabat | LC Kabat | LC Kabat |
|---|---|---|---|---|---|---|---|
| # | Name | CDR1 | CDR2 | CDR3 | CDR1 | CDR2 | CDR3 |
| 1 | PSMB889 | RYNMN | SINSN | TMGDY | TGSSF | DNSNR | QSYDS |
| | | | SRYIY | YYYYG | NLGAG | PS | SLSGV |
| | | | YADSV | MDV | YDVH | | V |
| | | | KG | | | | |
| | | 1 | 2 | 3 | 16 | 5 | 6 |
| 2 | PSMB890 | RYNMN | SINSN | TMGDY | AGSLS | GNINR | QSYDS |
| | | | SRYIY | YYYYG | NIGAG | LS | SLSSY |
| | | | YADSV | MDV | YDVH | | V |
| | | | KG | | | | |
| | | 1 | 2 | 3 | 38 | 39 | 40 |
| 3 | PSMB891 | TYGMH | VVSFD | ALRDG | SGSSS | SDNQR | AAWDD |
| | | | ESNKY | NNWDY | NIGSN | PS | SLNGY |
| | | | YADSV | FNGMD | TVN | | V |
| | | | KG | V | | | |
| | | 69 | 70 | 71 | 72 | 73 | 74 |
| 4 | PSMB892 | TYGMH | VVS FDESN | ALRDG | TGSSS | GNSNR | QSYDS |
| | | | KYYAD | NNWDY | NIGAD | PS | SLSGW |
| | | | SVKG | FNGMD | YDVH | | V |
| | | | | V | | | |
| | | 69 | 70 | 71 | 106 | 107 | 108 |
| 5 | PSMB893 | SYGIH | VIWYD | GVGPT | SGSSS | SDNQR | AAWDD |
| | | | GSNKY | SYYYN | NIGSN | PS | SLNGY |
| | | | YADSV | YGMDV | TVN | | V |
| | | | KG | | | | |
| | | 137 | 138 | 139 | 72 | 73 | 74 |
| 6 | PSMB894 | SYGIH | VIWYD | GVGPT | TGSSS | GNSNR | QSYDS |
| | | | GSNKY | SYYYN | NIGAD | PS | SLSGW |
| | | | YADSV | YGMDV | YDVH | | V |
| | | | KG | | | | |
| | | 137 | 138 | 139 | 106 | 107 | 108 |
| 7 | PSMB895 | SYAMS | AISGG | DGVGA | SGSSS | DNNKR | GTWDS |
| | | | IGSTY | TPYYF | NIGNN | PS | SLSAY |
| | | | YADSV | DY | YVS | | V |
| | | | KG | | | | |
| | | 205 | 206 | 207 | 208 | 209 | 210 |
| 8 | PSMB896 | SYAMS | AISGG | DGVGA | SGSSS | DNNKR | GTWDS |
| | | | IGSTY | TPYYF | NIGIN | PS | SLSAV |
| | | | YADSV | DY | YVS | | V |
| | | | KG | | | | |
| | | 205 | 206 | 207 | 242 | 209 | 244 |
| 9 | PSMB897 | SYAMS | AISGG | DGVGA | SGSSS | DNNKR | GTWDS |
| | | | SGSTY | TPYYF | NIGNN | PS | SLSAY |
| | | | YADSV | DY | YVS | | V |
| | | | KG | | | | |
| | | 205 | 274 | 207 | 208 | 209 | 210 |
| 10 | PSMB898 | SYAMS | AISGG | DGVGA | SGSSS | DNNKR | GTWDS |
| | | | SGSTY | TPYYF | NIGIN | PS | SLSAV |
| | | | YADSV | DY | YVS | | V |
| | | | KG | | | | |
| | | 205 | 274 | 207 | 242 | 209 | 244 |
| 11 | PSMB899 | SYAMS | AISGG | DGVGA | SGSSS | DNNKR | GTWDS |
| | | | YGAPY | TPYYF | NIGNN | PS | SLSAY |
| | | | YADTV | DD | YVS | | V |
| | | | KG | | | | |
| | | 205 | 342 | 343 | 208 | 209 | 210 |
| 12 | PSMHB49SC1133 | FYSMN | SISSS | SYSGS | RASQS | GASSR | QQYGV |
| | _011A11_1 | | GNYIY | YDAFD | VSSSF | AT | SPWT |
| | | | YADSV | E | LA | | |
| | | | KG | | | | |
| | | 375 | 376 | 377 | 378 | 379 | 380 |
| 13 | PSMB896-G100A | SYAMS | AISGG | DAVGA | SGSSS | DNNKR | GTWDS |
| | | | IGSTY | TPYYF | NIGIN | PS | SLSAV |
| | | | YADSV | DY | YVS | | V |
| | | | KG | | | | |
| | | 205 | 206 | 411 | 242 | 209 | 244 |
| 14 | PSMA_P72_ | SYNMN | IIYYD | ERGRD | SGDAL | KDSER | QSADS |
| | A10- | | ESNKY | YYGMD | PKQYA | PS | SGTYV |
| | HC-G54E | | YADSV | V | Y | | |
| | | | KG | | | | |
| | | 443 | 444 | 445 | 446 | 447 | 448 |
| 15 | PSMA_P72_D01- | NYNMN | HISTS | EGVGA | TGTSS | EVSNR | SSYTS |
| | HC-D95E | | SSNKY | DYGDY | DVGGY | PS | SYTYV |
| | | | YADSV | YYYGM | NYVS | | |
| | | | KG | DV | | | |
| | | 477 | 478 | 479 | 480 | 481 | 482 |
| 16 | PSMA_P72_F01 | TYGMH | FISYD | RDNLR | RASQS | GASTR | HQYND |
| | and | | GSNKY | FLEWE | VRSNL | AT | WPPYT |
| | PSMA_P75_F01 | | YADSV | MDV | A | | |
| | | | KG | | | | |
| | | 69 | 512 | 513 | 514 | 515 | 516 |
| 17 | PSMA_P72_F07 | SYGMN | VTSYD | DPYSS | GGNNI | DDSDR | QVWDS |
| | | | GSNKY | SWNGA | GSKSV | PS | STDHV |
| | | | YADSV | FDI | H | | V |
| | | | KG | | | | |
| | | 545 | 546 | 547 | 548 | 549 | 550 |
| 18 | PSMA_P72_E07 | TYGMH | VVSFD | ALRDG | SGSSS | SDNOR | AAWDD |
| | | | ESNKY | NNWDY | NIGSN | PS | SLNGY |
| | | | YADSV | FNGMD | TVN | | V |
| | | | KG | V | | | |
| | | 69 | 70 | 71 | 72 | 73 | 74 |
| 19 | PSMA_P72_D01 | NYNMN | HISTS | DGVGA | TGTSS | EVSNR | SSYTS |
| | | | SSNKY | DYGDY | DVGGY | PS | SYTYV |
| | | | YADSV | YYYGM | NYVS | | |
| | | | KG | DV | | | |
| | | 477 | 478 | 615 | 480 | 481 | 482 |
| 20 | PSMA_P72_C01 | GYGMH | VMSYD | DTVWG | GGNNS | DDSDR | QVWDS |
| | | | GSNRF | SHPDA | GSKSV | PS | SSDHG |
| | | | YVDSV | FDI | H | | V |
| | | | RG | | | | |
| | | 647 | 648 | 649 | 650 | 549 | 652 |
| 21 | PSMA_P72_A10 | SYNMN | IIYYD | ERGRD | SGDAL | KDSER | QSADS |
| | | | GSNKY | YYGMD | PKOYA | PS | SGTYV |
| | | | YADSV | V | Y | | |
| | | | KG | | | | |
| | | 443 | 682 | 445 | 446 | 447 | 448 |
| 22 | PSMA_P72_F02 | SYYWS | RIYSS | VGVWP | SGSSS | SSNQR | AAWDD |
| | and | | GSTNY | GAFDI | NIGSN | PS | SLNGV |
| | PSMA_P70_F02 | | NPSLK | | TVN | | V |
| | | | S | | | | |
| | | 715 | 716 | 717 | 72 | 719 | 720 |
| 23 | PSMA_P72_G02 | GYGMH | VISYD | DRIWG | TGASS | EVSNR | SSYTI |
| | | | GSNKY | SRGYY | DVGGY | PS | TSTLV |
| | | | YADSV | YGMDV | NYVS | | |
| | | | KG | | | | |
| | | 647 | 750 | 751 | 752 | 481 | 754 |
| 24 | PSMA_P72_A11 | GYGLH | LISYD | TTVSD | GGNNI | DDSDR | QVWDS |
| | | | GSNKY | PYYYG | GSKSV | PS | SSDHV |
| | | | YADSV | MDV | H | | V |
| | | | KG | | | | |
| | | 783 | 784 | 785 | 548 | 549 | 788 |

TABLE 6
PSMA Antibody Chothia CDR Amino Acid Sequences

| | | HC | HC | HC | LC | LC | LC |
|---|---|---|---|---|---|---|---|
| | | Chothia | Chothia | Chothia | Chothia | Chothia | Chothia |
| # | Protein Name | CDR1 | CDR2 | CDR3 | CDR1 | CDR2 | CDR3 |
| 1 | PSMB889 | GFTFSRY | NSNSRY | MGDYYYYYGMD | SSFNLGAGYD | DNS | YDSSLSGV |
| | | 7 | 4 | 9 | 10 | 11 | 12 |
| 2 | PSMB890 | GFTFSRY | NSNSRY | MGDYYYYYGMD | SLSNIGAGYD | GNI | YDSSLSSY |
| | | 7 | 4 | 9 | 44 | 45 | 46 |
| 3 | PSMB891 | GFTFITY | SFDESN | LRDGNNWDYFNGMD | SSSNIGSNT | SDN | WDDSLNGY |
| | | 75 | 76 | 77 | 78 | 79 | 80 |
| 4 | PSMB892 | GFTFITY | SFDESN | LRDGNNWDYENGMD | SSSNIGADYD | GNS | YDSSLSGW |
| | | 75 | 76 | 77 | 112 | 113 | 114 |
| 5 | PSMB893 | GFTESSY | WYDGSN | VGPTSYYYNYGMD | SSSNIGSNT | SDN | WDDSLNGY |
| | | 143 | 144 | 145 | 78 | 79 | 80 |
| 6 | PSMB894 | GFTESSY | WYDGSN | VGPTSYYYNYGMD | SSSNIGADYD | GNS | YDSSLSGW |
| | | 143 | 144 | 145 | 112 | 113 | 114 |
| 7 | PSMB895 | GFTESSY | SGGIGS | GVGATPYYED | SSSNIGNNY | DNN | WDSSLSAY |
| | | 143 | 212 | 213 | 214 | 215 | 216 |
| 8 | PSMB896 | GFTFSSY | SGGIGS | GVGATPYYFD | SSSNIGINY | DNN | WDSSLSAV |
| | | 143 | 212 | 213 | 248 | 215 | 250 |
| 9 | PSMB897 | GFTFSSY | SGGSGS | GVGATPYYED | SSSNIGNNY | DNN | WDSSLSAY |
| | | 143 | 280 | 213 | 214 | 215 | 216 |
| 10 | PSMB898 | GFTESSY | SGGSGS | GVGATPYYFD | SSSNIGINY | DNN | WDSSLSAV |
| | | 143 | 280 | 213 | 248 | 215 | 250 |
| 11 | PSMB899 | GFIFSSY | SGGYGA | GVGATPYYFD | SSSNIGNNY | DNN | WDSSLSAY |
| | | 347 | 348 | 213 | 214 | 215 | 216 |
| 12 | PSMHB49SC1133 | GFTFSFY | SSSGNY | SYSGSYDAFD | SQSVSSSF | GAS | YGVSPW |
| | _011A11_1 | 381 | 382 | 383 | 384 | 385 | 386 |
| 13 | PSMB896- | GFTESSY | SGGIGS | DAVGATPYYED | SSSNIGINY | DNN | WDSSLSAV |
| | G100A | 143 | 212 | 417 | 248 | 215 | 250 |
| 14 | PSMA_P72_A10- | GFTESSY | YYDESN | ERGRDYYGMD | DALPKQY | KDS | ADSSGTY |
| | HC-G54E | 143 | 450 | 451 | 452 | 453 | 454 |
| 15 | PSMA_P72_D01- | GFTENNY | STSSSN | EGVGADYGDYYYYGMD | TSSDVGGYNY | EVS | YTSSYTY |
| | HC-D95E | 483 | 484 | 485 | 486 | 487 | 488 |
| 16 | PSMA_P72_F01 | GFTESTY | SYDGSN | RDNLRFLEWEMD | SQSVRSN | GAS | YNDWPPY |
| | and | | | | | | |
| | PSMA_P75_F01 | | | | | | |
| | | 517 | 518 | 519 | 520 | 385 | 522 |
| 17 | PSMA_P72_F07 | GFTESSY | SYDGSN | DPYSSSWNGAFD | NNIGSKS | DDS | WDSSTDHV |
| | | 143 | 518 | 553 | 554 | 555 | 556 |
| 18 | PSMA_P72_E07 | GFTFITY | SFDESN | ALRDGNNWDYFNGMD | SSSNIGSNT | SDN | WDDSLNGY |
| | | 75 | 76 | 587 | 78 | 79 | 80 |
| 19 | PSMA_P72_D01 | GFTENNY | STSSSN | DGVGADYGDYYYYGMD | TSSDVGGYNY | EVS | YTSSYTY |
| | | 483 | 484 | 621 | 486 | 487 | 488 |
| 20 | PSMA_P72_C01 | GFSFSGY | SYDGSN | DTVWGSHPDAFD | NNSGSKS | DDS | WDSSSDHG |
| | | 653 | 518 | 655 | 656 | 555 | 658 |
| 21 | PSMA_P72_A10 | GFTESSY | YYDGSN | ERGRDYYGMD | DALPKQY | KDS | ADSSGTY |
| | | 143 | 688 | 451 | 452 | 453 | 454 |
| 22 | PSMA_P72_F02 | GGSIISY | YSSGS | VGVWPGAFD | SSSNIGSNT | SSN | WDDSLNGV |
| | and | | | | | | |
| | PSMA_P70_F02 | | | | | | |
| | | 721 | 722 | 723 | 78 | 725 | 726 |
| 23 | PSMA_P72_G02 | GFSFSGY | SYDGSN | DRIWGSRGYYYGMD | ASSDVGGYNY | EVS | YTITSTL |
| | | 653 | 518 | 757 | 758 | 487 | 760 |
| 24 | PSMA_P72_A11 | GFSFSGY | SYDGSN | TTVSDPYYYGMD | NNIGSKS | DDS | WDSSSDHV |
| | | 653 | 518 | 791 | 554 | 555 | 794 |

TABLE 7
PSMA Antibody AbM CDR Amino Acid Sequences

| | | HC AbM | HC AbM | | | LC AbM | |
|---|---|---|---|---|---|---|---|
| # | Protein Name | CDR1 | CDR2 | HC AbM CDR3 | LC AbM CDR1 | CDR2 | LC AbM CDR3 |
| 1 | PSMB889 | GFTFSRYNMN | SINSNSRYIY | TMGDYYYYYGMDV | TGSSFNLGAGYDVH | DNSNRPS | QSYDSSLSGVV |
| | | 13 | 14 | 3 | 16 | 5 | 6 |
| 2 | PSMB890 | GFTFSRYNMN | SINSNSRYIY | TMGDYYYYYGMDV | AGSLSNIGAGYDVH | GNINRLS | QSYDSSLSSYV |
| | | 13 | 14 | 3 | 38 | 39 | 40 |
| 3 | PSMB891 | GFTFITYGMH | VVSFDESNKY | ALRDGNNWDYFNGMDV | SGSSSNIGSNTVN | SDNORPS | AAWDDSLNGYV |
| | | 81 | 82 | 71 | 72 | 73 | 74 |
| 4 | PSMB892 | GFTFITYGMH | VVSFDESNKY | ALRDGNNWDYFNGMDV | TGSSSNIGADYDVH | GNSNRPS | QSYDSSLSGWV |
| | | 81 | 82 | 71 | 106 | 107 | 108 |
| 5 | PSMB893 | GFTFSSYGIH | VIWYDGSNKY | GVGPTSYYYNYGMDV | SGSSSNIGSNTVN | SDNQRPS | AAWDDSLNGYV |
| | | 149 | 150 | 139 | 72 | 73 | 74 |
| 6 | PSMB894 | GFTFSSYGIH | VIWYDGSNKY | GVGPTSYYYNYGMDV | TGSSSNIGADYDVH | GNSNRPS | QSYDSSLSGWV |
| | | 149 | 150 | 139 | 106 | 107 | 108 |
| 7 | PSMB895 | GFTFSSYAMS | AISGGIGSTY | DGVGATPYYFDY | SGSSSNIGNNYVS | DNNKRPS | GTWDSSLSAYV |
| | | 217 | 218 | 207 | 208 | 209 | 210 |
| 8 | PSMB896 | GFTFSSYAMS | AISGGIGSTY | DGVGATPYYFDY | SGSSSNIGINYVS | DNNKRPS | GTWDSSLSAVV |
| | | 217 | 218 | 207 | 242 | 209 | 244 |
| 9 | PSMB897 | GFTFSSYAMS | AISGGSGSTY | DGVGATPYYFDY | SGSSSNIGNNYVS | DNNKRPS | GTWDSSLSAYV |
| | | 217 | 286 | 207 | 208 | 209 | 210 |
| 10 | PSMB898 | GFTESSYAMS | AISGGSGSTY | DGVGATPYYFDY | SGSSSNIGINYVS | DNNKRPS | GTWDSSLSAVV |
| | | 217 | 286 | 207 | 242 | 209 | 244 |
| 11 | PSMB899 | GFIFSSYAMS | AISGGYGAPY | DGVGATPYYFDD | SGSSSNIGNNYVS | DNNKRPS | GTWDSSLSAYV |
| | | 353 | 354 | 343 | 208 | 209 | 210 |
| 12 | PSMHB49SC113 | GFTFSFYSMN | SISSSGNYIY | SYSGSYDAFDE | RASQSVSSSFLA | GASSRAT | QQYGVSPWT |
| | 3_011A11_1 | | | | | | |
| | | 387 | 388 | 377 | 378 | 379 | 380 |
| 13 | PSMB896- | GFTFSSYAMS | AISGGIGSTY | DAVGATPYYFDY | SGSSSNIGINYVS | DNNKRPS | GTWDSSLSAVV |
| | G100A | | | | | | |
| | | 217 | 218 | 411 | 242 | 209 | 244 |
| 14 | PSMA_P72_A10 | GFTFSSYNMN | IIYYDESNKY | ERGRDYYGMDV | SGDALPKQYAY | KDSERPS | QSADSSGTYV |
| | -HC-G54E | | | | | | |
| | | 455 | 456 | 445 | 446 | 447 | 448 |
| 15 | PSMA_P72_D01 | GFTENNYNMN | HISTSSSNKY | EGVGADYGDYYYYGMDV | TGTSSDVGGYNYVS | EVSNRPS | SSYTSSYTYV |
| | -HC-D95E | | | | | | |
| | | 489 | 490 | 479 | 480 | 481 | 482 |
| 16 | PSMA_P72_F01 | GFTFSTYGMH | FISYDGSNKY | RDNLRFLEWFMDV | RASQSVRSNLA | GASTRAT | HQYNDWPPYT |
| | and | | | | | | |
| | PSMA_P75_F01 | | | | | | |
| | | 523 | 524 | 513 | 514 | 515 | 516 |
| 17 | PSMA_P72_F07 | GFTFSSYGMN | VTSYDGSNKY | DPYSSSWNGAFDI | GGNNIGSKSVH | DDSDRPS | QVWDSSTDHVV |
| | | 557 | 558 | 547 | 548 | 549 | 550 |
| 18 | PSMA_P72_E07 | GFTFITYGMH | VVSFDESNKY | ALRDGNNWDYFNGMDV | SGSSSNIGSNTVN | SDNORPS | AAWDDSLNGYV |
| | | 81 | 82 | 71 | 72 | 73 | 74 |
| 19 | PSMA_P72_D01 | GFTENNYNMN | HISTSSSNKY | DGVGADYGDYYYYGMDV | TGTSSDVGGYNYVS | EVSNRPS | SSYTSSYTYV |
| | | 489 | 490 | 615 | 480 | 481 | 482 |
| 20 | PSMA_P72_C01 | GFSFSGYGMH | VMSYDGSNRF | DTVWGSHPDAFDI | GGNNSGSKSVH | DDSDRPS | QVWDSSSDHGV |
| | | 659 | 660 | 649 | 650 | 549 | 652 |
| 21 | PSMA_P72_A10 | GFTESSYNMN | IIYYDGSNKY | ERGRDYYGMDV | SGDALPKQYAY | KDSERPS | QSADSSGTYV |
| | | 455 | 694 | 445 | 446 | 447 | 448 |
| 22 | PSMA_P72_F02 | GGSIISYYWS | RIYSSGSTN | VGVWPGAFDI | SGSSSNIGSNTVN | SSNQRPS | AAWDDSLNGVV |
| | and | | | | | | |
| | PSMA_P70_F02 | | | | | | |
| | | 727 | 728 | 717 | 72 | 719 | 720 |
| 23 | PSMA_P72_G02 | GFSFSGYGMH | VISYDGSNKY | DRIWGSRGYYYGMDV | TGASSDVGGYNYVS | EVSNRPS | SSYTITSTLV |
| | | 659 | 762 | 751 | 752 | 481 | 754 |
| 24 | PSMA_P72_A11 | GFSFSGYGLH | LISYDGSNKY | TTVSDPYYYGMDV | GGNNIGSKSVH | DDSDRPS | QVWDSSSDHVV |
| | | 795 | 796 | 785 | 548 | 549 | 788 |

TABLE 8
PSMA Antibody Contact CDR Amino Acid Sequences

| # | Protein | HC Contact | HC Contact | HC Contact | LC Contact | LC Contact | LC Contact |
|---|---|---|---|---|---|---|---|
| | Name | CDR1 | CDR2 | CDR3 | CDR1 | CDR2 | CDR3 |
| 1 | PSMB889 | SRYNMN | WVSSINSNSRYIY | AKTMGDYYYYYGMD | LGAGYDVHWY | LLIYDNSNRP | QSYDSSLSGV |
| | | 19 | 20 | 21 | 22 | 23 | 24 |
| 2 | PSMB890 | SRYNMN | WVSSINSNSRYIY | AKTMGDYYYYYGMD | IGAGYDVHWY | LLIYGNINRL | QSYDSSLSSY |
| | | 19 | 20 | 21 | 56 | 57 | 58 |
| 3 | PSMB891 | ITYGMH | WVAVVS FDESNKY | ARALRDGNNWDYENGMD | IGSNTVNWF | LLIYSDNQRP | AAWDDSLNGY |
| | | 87 | 88 | 89 | 90 | 91 | 92 |
| 4 | PSMB892 | ITYGMH | WVAVVSFDESNKY | ARALRDGNNWDYENGMD | IGADYDVHWY | LLIYGNSNRP | QSYDSSLSGW |
| | | 87 | 88 | 89 | 124 | 125 | 126 |
| 5 | PSMB893 | SSYGIH | WVAVIWYDGSNKY | VRGVGPTSYYYNYGMD | IGSNTVNWF | LLIYSDNQRP | AAWDDSLNGY |
| | | 155 | 156 | 157 | 90 | 91 | 92 |
| 6 | PSMB894 | SSYGIH | WVAVIWYDGSNKY | VRGVGPTSYYYNYGMD | IGADYDVHWY | LLIYGNSNRP | QSYDSSLSGW |
| | | 155 | 156 | 157 | 124 | 125 | 126 |
| 7 | PSMB895 | SSYAMS | WVSAISGGIGSTY | AKDGVGATPYYED | IGNNYVSWY | LLIYDNNKRP | GTWDSSLSAY |
| | | 223 | 224 | 225 | 226 | 227 | 228 |
| 8 | PSMB896 | SSYAMS | WVSAISGGIGSTY | AKDGVGATPYYED | IGINYVSWY | LLIYDNNKRP | GTWDSSLSAV |
| | | 223 | 224 | 225 | 260 | 227 | 262 |
| 9 | PSMB897 | SSYAMS | WVSAISGGSGSTY | AKDGVGATPYYED | IGNNYVSWY | LLIYDNNKRP | GTWDSSLSAY |
| | | 223 | 292 | 225 | 226 | 227 | 228 |
| 10 | PSMB898 | SSYAMS | WVSAISGGSGSTY | AKDGVGATPYYFD | IGINYVSWY | LLIYDNNKRP | GTWDSSLSAV |
| | | 223 | 292 | 225 | 260 | 227 | 262 |
| 11 | PSMB899 | SSYAMS | WVSAISGGYGAPY | AKDGVGATPYYFD | IGNNYVSWY | LLIFDNNKRP | GTWDSSLSAY |
| | | 223 | 360 | 225 | 226 | 363 | 228 |
| 12 | PSMHB49SC1133_ | SFYSMN | WVSSISSSGNYIY | ARSYSGSYDAFD | SSSFLAWY | LLISGASSRA | QQYGVS PW |
| | 011A11_1 | | | | | | |
| | | 393 | 394 | 395 | 396 | 397 | 398 |
| 13 | PSMB896-G100A | SSYAMS | WVSAISGGIGSTY | AKDAVGATPYYED | IGINYVSWY | LLIYDNNKRP | GTWDSSLSAV |
| | | 223 | 224 | 429 | 260 | 227 | 262 |
| 14 | PSMA_P72_A10- | SSYNMN | WVAIIYYDESNKY | ARERGRDYYGMD | KQYAYWY | LVIYKDSERP | QSADSSGTY |
| | HC-G54E | | | | | | |
| | | 461 | 462 | 463 | 464 | 465 | 466 |
| 15 | PSMA_P72_D01- | NNYNMN | WVSHISTSSSNKY | AREGVGADYGDYYYYGMD | VGGYNYVSWY | LMIYEVSNRP | SSYTSSYTY |
| | HC-D95E | | | | | | |
| | | 495 | 496 | 497 | 498 | 499 | 500 |
| 16 | PSMA_P72_F01 | STYGMH | WVAFISYDGSNKY | AGRDNLRFLEWFMD | RSNLAWY | LLIYGASTRA | HQYNDWPPY |
| | and | | | | | | |
| | PSMA_P75_F01 | | | | | | |
| | | 529 | 530 | 531 | 32 | 533 | 534 |
| 17 | PSMA_P72_F07 | SSYGMN | WVAVTSYDGSNKY | ARDPYSSSWNGAFD | SKSVHWY | LVVYDDSDRP | QVWDSSTDHV |
| | | 563 | 564 | 565 | 566 | 567 | 568 |
| 18 | PSMA_P72_E07 | ITYGMH | WVAVVS FDESNKY | ARALRDGNNWDYFNGMD | IGSNTVNWF | LLIYSDNQRP | AAWDDSLNGY |
| | | 87 | 88 | 89 | 90 | 91 | 92 |
| 19 | PSMA_P72_D01 | NNYNMN | WVSHISTSSSNKY | ARDGVGADYGDYYYYGMD | VGGYNYVSWY | LMIYEVSNRP | SSYTSSYTY |
| | | 495 | 496 | 633 | 498 | 499 | 500 |
| 20 | PSMA_P72_C01 | SGYGMH | WVAVMSYDGSNRF | ARDTVWGSHPDAFD | SKSVHWY | LVVYDDSDRP | QVWDSSSDHG |
| | | 665 | 666 | 667 | 566 | 567 | 670 |
| 21 | PSMA_P72_A10 | SSYNMN | WVAIIYYDGSNKY | ARERGRDYYGMD | KOYAYWY | LVIYKDSERP | QSADSSGTY |
| | | 461 | 700 | 463 | 464 | 465 | 466 |
| 22 | PSMA_P72_F02 | ISYYWS | WIGRIYSSGSTN | AKVGVWPGAFD | IGSNTVNWY | LLIYSSNQRP | AAWDDSLNGV |
| | and | | | | | | |
| | PSMA_P70_F02 | | | | | | |
| | | 733 | 734 | 735 | 736 | 737 | 738 |
| 23 | PSMA_P72_G02 | SGYGMH | WVAVISYDGSNKY | ARDRIWGSRGYYYGMD | VGGYNYVSWY | LMIYEVSNRP | SSYTITSTL |
| | | 665 | 768 | 769 | 498 | 499 | 772 |
| 24 | PSMA_P72_A11 | SGYGLH | WVTLISYDGSNKY | AKTTVSDPYYYGMD | SKSVHWY | LVVYDDSDRP | QVWDSSSDHV |
| | | 801 | 802 | 803 | 566 | 567 | 806 |

TABLE 9
PSMA Antibody IMGT CDR Amino Acid Sequences

| | | | | | | LC | |
|---|---|---|---|---|---|---|---|
| | Protein | HC IMGT | HC IMGT | HC IMGT | LC IMGT | IMGT | LC IMGT |
| # | Name | CDR1 | CDR2 | CDR3 | CDR1 | CDR2 | CDR3 |
| 1 | PSMB889 | GFTFSRYN | INSNSRYI | AKTMGDYYYYYGMDV | SFNLGAGYD | DNS | QSYDSSLSGVV |
| | | 25 | 26 | 27 | 28 | 11 | 6 |
| 2 | PSMB890 | GFTFSRYN | INSNSRYI | AKTMGDYYYYYGMDV | LSNIGAGYD | GNI | QSYDSSLSSYV |
| | | 25 | 26 | 27 | 62 | 45 | 40 |
| 3 | PSMB891 | GFTFITYG | VSFDESNK | ARALRDGNNWDYFNGMDV | SSNIGSNT | SDN | AAWDDSLNGYV |
| | | 93 | 94 | 95 | 96 | 79 | 74 |
| 4 | PSMB892 | GFTFITYG | VSFDESNK | ARALRDGNNWDYFNGMDV | SSNIGADYD | GNS | QSYDSSLSGWV |
| | | 93 | 94 | 95 | 130 | 113 | 108 |
| 5 | PSMB893 | GFTFSSYG | IWYDGSNK | VRGVGPTSYYYNYGMDV | SSNIGSNT | SDN | AAWDDSLNGYV |
| | | 161 | 162 | 163 | 96 | 79 | 74 |
| 6 | PSMB894 | GFTFSSYG | IWYDGSNK | VRGVGPTSYYYNYGMDV | SSNIGADYD | GNS | QSYDSSLSGWV |
| | | 161 | 162 | 163 | 130 | 113 | 108 |
| 7 | PSMB895 | GFTFSSYA | ISGGIGST | AKDGVGATPYYFDY | SSNIGNNY | DNN | GTWDSSLSAYV |
| | | 229 | 230 | 231 | 232 | 215 | 210 |
| 8 | PSMB896 | GFTESSYA | ISGGIGST | AKDGVGATPYYFDY | SSNIGINY | DNN | GTWDSSLSAVV |
| | | 229 | 230 | 231 | 266 | 215 | 244 |
| 9 | PSMB897 | GFTFSSYA | ISGGSGST | AKDGVGATPYYFDY | SSNIGNNY | DNN | GTWDSSLSAYV |
| | | 229 | 298 | 231 | 232 | 215 | 210 |
| 10 | PSMB898 | GFTESSYA | ISGGSGST | AKDGVGATPYYFDY | SSNIGINY | DNN | GTWDSSLSAVV |
| | | 229 | 298 | 231 | 266 | 215 | 244 |
| 11 | PSMB899 | GFIFSSYA | ISGGYGAP | AKDGVGATPYYFDD | SSNIGNNY | DNN | GTWDSSLSAYV |
| | | 365 | 366 | 367 | 232 | 215 | 210 |
| 12 | PSMHB49SC1133_ | GFTFSFYS | ISSSGNYI | ARSYSGSYDAFDF | QSVSSSF | GAS | QQYGVSPWT |
| | 011A11_1 | | | | | | |
| | | 399 | 400 | 401 | 402 | 385 | 380 |
| 13 | PSMB896-G100A | GFTFSSYA | ISGGIGST | AKDAVGATPYYEDY | SSNIGINY | DNN | GTWDSSLSAVV |
| | | 229 | 230 | 435 | 266 | 215 | 244 |
| 14 | PSMA_P72_A10- | GFTFSSYN | IYYDESNK | ARERGRDYYGMDV | ALPKQY | KDS | QSADSSGTYV |
| | HC-G54E | | | | | | |
| | | 467 | 468 | 469 | 470 | 453 | 448 |
| 15 | PSMA_P72_D01- | GFTENNYN | ISTSSSNK | AREGVGADYGDYYYYGMDV | SSDVGGYNY | EVS | SSYTSSYTYV |
| | HC-D95E | | | | | | |
| | | 501 | 502 | 503 | 504 | 487 | 482 |
| 16 | PSMA_P72_F01 | GFTFSTYG | ISYDGSNK | AGRDNLRFLEWFMDV | QSVRSN | GAS | HQYNDWPPYT |
| | and | | | | | | |
| | PSMA_P75_F01 | | | | | | |
| | | 535 | 536 | 537 | 538 | 385 | 516 |
| 17 | PSMA_P72_F07 | GFTFSSYG | TSYDGSNK | ARDPYSSSWNGAFDI | NIGSKS | DDS | QVWDSSTDHVV |
| | | 161 | 570 | 571 | 572 | 555 | 550 |
| 18 | PSMA_P72_E07 | GFTFITYG | VSFDESNK | ARALRDGNNWDYFNGMDV | SSNIGSNT | SDN | AAWDDSLNGYV |
| | | 93 | 94 | 95 | 96 | 79 | 74 |
| 19 | PSMA_P72_D01 | GFTENNYN | ISTSSSNK | ARDGVGADYGDYYYYGMDV | SSDVGGYNY | EVS | SSYTSSYTYV |
| | | 501 | 502 | 639 | 504 | 487 | 482 |
| 20 | PSMA_P72_C01 | GFSFSGYG | MSYDGSNR | ARDTVWGSHPDAFDI | NSGSKS | DDS | QVWDSSSDHGV |
| | | 671 | 672 | 673 | 674 | 555 | 652 |
| 21 | PSMA_P72_A10 | GFTFSSYN | IYYDGSNK | ARERGRDYYGMDV | ALPKQY | KDS | QSADSSGTYV |
| | | 467 | 706 | 469 | 470 | 453 | 448 |
| 22 | PSMA_P72_F02 | GGSIISYY | IYSSGST | AKVGVWPGAFDI | SSNIGSNT | SSN | AAWDDSLNGVV |
| | and | | | | | | |
| | PSMA_P70_F02 | | | | | | |
| | | 739 | 740 | 741 | 96 | 725 | 720 |
| 23 | PSMA_P72_G02 | GFSFSGYG | ISYDGSNK | ARDRIWGSRGYYYGMDV | SSDVGGYNY | EVS | SSYTITSTLV |
| | | 671 | 536 | 775 | 504 | 487 | 754 |
| 24 | PSMA_P72_A11 | GFSFSGYG | ISYDGSNK | AKTTVSDPYYYGMDV | NIGSKS | DDS | QVWDSSSDHVV |
| | | 671 | 536 | 809 | 572 | 555 | 788 |

Exemplary PSMA binders in scFv format LH (VL-Linker-VH) or HL (VH-Linker-VL) are provided below.

TABLE 10
PSMA binders in scFv format LH (VL-Linker-VH) or HL (VH-Linker-VL)
using the Linker of SEQ ID NO: 1419

| | PSMA binders in scFv format LH (VL-Linker-VH) |
|---|---|
| | or HL (VH-Linker-VL) |
| PSMB895 scFv | QSVLTQPPSVSAAPGQKVTISCSGSSSNIGNNYVSWYQQLPGTAPKLLIYDNNKRPSGI |
| LH | PDRFSGSKSGTSATLGITGLQTGDEADYYCGTWDSSLSAYVFGTGTKVTVLGGSEGKSS |
| | GSGSESKSTGGSEVQLVESGGGLVQPGGSLRLSCAASGFTFSSYAMSWVRQAPGKGLEW |
| | VSAISGGIGSTYYADSVKGRFTISRDNSKNTLWLQMNSLRAEDTAVYYCAKDGVGATPY |
| | YFDYWGQGTLVTVSS |
| | (SEQ ID NO: 1485) |
| PSMB896 scFv | QSVLTQPPSVSAAPGQKVTISCSGSSSNIGINYVSWYQQLPGTAPKLLIYDNNKRPSGI |
| LH | PDRFSGSKSGTSATLGITGLQTGDEADYYCGTWDSSLSAVVFGGGTKLTVLGGSEGKSS |
| | GSGSESKSTGGSEVQLVESGGGLVQPGGSLRLSCAASGFTFSSYAMSWVRQAPGKGLEW |
| | VSAISGGIGSTYYADSVKGRFTISRDNSKNTLWLQMNSLRAEDTAVYYCAKDGVGATPY |
| | YFDYWGQGTLVTVSS |
| | (SEQ ID NO: 1486) |
| PSMB897 scFv | QSVLTQPPSVSAAPGQKVTISCSGSSSNIGNNYVSWYQQLPGTAPKLLIYDNNKRPSGI |
| LH | PDRFSGSKSGTSATLGITGLQTGDEADYYCGTWDSSLSAYVFGTGTKVTVLGGSEGKSS |
| | GSGSESKSTGGSEVQLVESGGGLVQPGGSLRLSCAASGFTFSSYAMSWVRQAPGKGLEW |
| | VSAISGGSGSTYYADSVKGRFTISRDNSKNTLYLQMNSLRAEDSAVYYCAKDGVGATPY |
| | YFDYWGQGTLVTVSS |
| | (SEQ ID NO: 1487) |
| PSMB898 scFb | QSVLTQPPSVSAAPGQKVTISCSGSSSNIGINYVSWYQQLPGTAPKLLIYDNNKRPSGI |
| LH | PDRFSGSKSGTSATLGITGLQTGDEADYYCGTWDSSLSAVVFGGGTKLTVLGGSEGKSS |
| | GSGSESKSTGGSEVQLVESGGGLVQPGGSLRLSCAASGFTESSYAMSWVRQAPGKGLEW |
| | VSAISGGSGSTYYADSVKGRFTISRDNSKNTLYLQMNSLRAEDSAVYYCAKDGVGATPY |
| | YFDYWGQGTLVTVSS |
| | (SEQ ID NO: 1488) |
| PSMA_P75_F01 | EIVLTQSPGTLSVSPGERATLSCRASQSVRSNLAWYQQKPGQAPRLLIYGASTRATGIP |
| scFv LH | ARFSGSGSGTEFTLTISSLQSEDFAVYYCHQYNDWPPYTFGQGTKLEIKGGSEGKSSGS |
| | GSESKSTGGSQVQLQESGGGVVQPGRSLRLSCAASGFTFSTYGMHWVRQAPGKGLEWVA |
| | FISYDGSNKYYADSVKGRFTISRDNSKHTLYLQMNSLRAEDTAVYYCAGRDNLRFLEWF |
| | MDVWGQGTTVTVSS |
| | (SEQ ID NO: 1489) |
| PSMA P70 F02 | QSVLTQPPSASGTPGQRVTISCSGSSSNIGSNTVNWYQQLPGTAPKLLIYSSNQRPSGV |
| scFv LH | PDRFSGSKSGTSASLAISGLQSEDEADYYCAAWDDSLNGVVFGGGTKLTVLGGSEGKSS |
| | GSGSESKSTGGSEVQLLESGPGLVKPSETLSLTCTVSGGSIISYYWSWIRQPAGKGLEW |
| | IGRIYSSGSTNYNPSLKSRVTMSVDTSKNQFSLKLSSVTAADTAVYYCAKVGVWPGAFD |
| | IWGQGTMVTVSS |
| | (SEQ ID NO: 1490) |
| PSMA_P72_A10- | SYELMQPPSVSVSPGQTARITCSGDALPKQYAYWYQQKPGQAPVLVIYKDSERPSGIPV |
| HC-G54E scFv | RFSGSSSGTTVTLTITGVQAEDEADYYCQSADSSGTYVFGTGTKVTVLGGSEGKSSGSG |
| LH | SESKSTGGSQVQLVESGGGVVQPGRSLRLSCAASGFTFSSYNMNWVRQAPGKGLEWVAI |
| | IYYDESNKYYADSVKGRFTISRDISKNTLYLQMNSLRAEDTAVYYCARERGRDYYGMDV |
| | WGQGTTVTVSS |
| | (SEQ ID NO: 1491) |
| PSMA_P72_D01- | QSVLTQPASVSGSPGQSITISCTGTSSDVGGYNYVSWYQQHPGKAPKLMIYEVSNRPSG |
| HC-D95E scFv | VSNRFSGSKSGNTASLTISGLQAEDEADYYCSSYTSSYTYVFGTGTKLTVLGGSEGKSS |
| LH | GSGSESKSTGGSEVQLVESGGDLVQPGGSLRLSCAASGFTENNYNMNWVRQAPGKGLEW |
| | VSHISTSSSNKYYADSVKGRESISRDIAKNSMYLQMNSLRDEDTAVYYCAREGVGADYG |
| | DYYYYGMDVWGQGTTVTVSS |
| | (SEQ ID NO: 1492) |
| PSMA_P72_F07 | SYELTQPPSVSVAPGQTARITCGGNNIGSKSVHWYQQKPGQAPVLVVYDDSDRPSGIPE |
| scFv LH | RFSGSNSGNTATLTISRVEAGDEADYYCQVWDSSTDHVVFGGGTKLTVLGGSEGKSSGS |
| | GSESKSTGGSEVQLVESGGGVVQPGRSLRLSCAASGFTFSSYGMNWVRQAPGKGLEWVA |
| | VTSYDGSNKYYADSVKGRFTISRDISKNTLYLQMSSLRAEDTAVYYCARDPYSSSWNGA |
| | FDIWGPGTMVTVSS |
| | (SEQ ID NO: 1493) |
| PSMA_P72_E07 | QSVLTQPPSASGTPGQGVTISCSGSSSNIGSNTVNWFQQLPGTAPKLLIYSDNQRPSGV |
| scFv LH | PDRESGSKSGTSASLAISGLQSEDEADYYCAAWDDSLNGYVFGTGTKVTVLGGSEGKSS |
| | GSGSESKSTGGSEVQLVESGGGVVQPGRSLRLSCAASGFTFITYGMHWVRQAPGKGLEW |
| | VAVVSFDESNKYYADSVKGRFTISRDNSKNTLYLQMNSLRAEDTAVYYCARALRDGNNW |
| | DYFNGMDVWGQGTTVTVSS |
| | (SEQ ID NO: 1494) |
| PSMA_P72_D01 | QSVLTQPASVSGSPGQSITISCTGTSSDVGGYNYVSWYQQHPGKAPKLMIYEVSNRPSG |
| scFv LH | VSNRFSGSKSGNTASLTISGLQAEDEADYYCSSYTSSYTYVFGTGTKLTVLGGSEGKSS |
| | GSGSESKSTGGSEVQLVESGGDLVQPGGSLRLSCAASGFTENNYNMNWVRQAPGKGLEW |
| | VSHISTSSSNKYYADSVKGRESISRDIAKNSMYLQMNSLRDEDTAVYYCARDGVGADYG |
| | DYYYYGMDVWGQGTTVTVSS |
| | (SEQ ID NO: 1495) |
| PSMA_P72_C01 | QSVLTQPPSVSVAPGQTARITCGGNNSGSKSVHWYQQKPGQAPVLVVYDDSDRPSGIPE |
| scFv LH | RESGSNSGNTATLTISRVEAGDEADYYCQVWDSSSDHGVFGGGTKLTVLGGSEGKSSGS |
| | GSESKSTGGSQVQLVESGGGEVQPGRSLRLSCAASGFSFSGYGMHWVRQAPGKGLEWVA |
| | VMSYDGSNRFYVDSVRGRESISRDNSKNTLYLQMNSLRPEDTAVYYCARDTVWGSHPDA |
| | FDIWGQGTVVTVSS |
| | (SEQ ID NO: 1496) |
| PSMA_P72_A10 | SYELMQPPSVSVSPGQTARITCSGDALPKQYAYWYQQKPGQAPVLVIYKDSERPSGIPV |
| scFv LH | RFSGSSSGTTVTLTITGVQAEDEADYYCQSADSSGTYVFGTGTKVTVLGGSEGKSSGSG |
| | SESKSTGGSQVQLVESGGGVVQPGRSLRLSCAASGFTFSSYNMNWVRQAPGKGLEWVAI |
| | IYYDGSNKYYADSVKGRFTISRDISKNTLYLQMNSLRAEDTAVYYCARERGRDYYGMDV |
| | WGQGTTVTVSS |
| | (SEQ ID NO: 1497) |
| PSMA_P70_F02 | QSVLTQPPSASGTPGQRVTISCSGSSSNIGSNTVNWYQQLPGTAPKLLIYSSNQRPSGV |
| scFv LH | PDRFSGSKSGTSASLAISGLQSEDEADYYCAAWDDSLNGVVFGGGTKLTVLGGSEGKSS |
| | GSGSESKSTGGSEVQLLESGPGLVKPSETLSLTCTVSGGSIISYYWSWIRQPAGKGLEW |
| | IGRIYSSGSTNYNPSLKSRVTMSVDTSKNQFSLKLSSVTAADTAVYYCAKVGVWPGAFD |
| | IWGQGTMVTVSS |
| | (SEQ ID NO: 1498) |
| PSMA_P72_G02 | EVQLVESGGGVVQPGRSLRLSCAASGFSFSGYGMHWVRQAPGKGLEWVAVISYDGSNKY |
| scFv HL | YADSVKGRFTISRDNSKNTLYLQMNSLRVEDTAVYYCARDRIWGSRGYYYGMDVWGQGT |
| | TVTVSSGGSEGKSSGSGSESKSTGGSQSALTQPASVSGSPGQSITISCTGASSDVGGYN |
| | YVSWYQQHPGKAPKLMIYEVSNRPSGVSNRFSGSKSGNTASLTISGLQAEDEADYYCSS |
| | YTITSTLVFGGGTKLTVL |
| | (SEQ ID NO: 1499) |
| PSMA_P72_C01 | QVQLVESGGGEVQPGRSLRLSCAASGFSFSGYGMHWVRQAPGKGLEWVAVMSYDGSNRF |
| scFv HL | YVDSVRGRESISRDNSKNTLYLQMNSLRPEDTAVYYCARDTVWGSHPDAFDIWGQGTVV |
| | TVSSGGSEGKSSGSGSESKSTGGSQSVLTQPPSVSVAPGQTARITCGGNNSGSKSVHWY |
| | QQKPGQAPVLVVYDDSDRPSGIPERFSGSNSGNTATLTISRVEAGDEADYYCQVWDSSS |
| | DHGVFGGGTKLTVL |
| | (SEQ ID NO: 1500) |
| PSMA_P72_A11 | QVQLQESGGDVVQPGRSLRLSCAASGESFSGYGLHWVRQAPGRGLEWVTLISYDGSNKY |
| scFv HL | YADSVKGRFTISRDNSKNTLYLQMNSLRAEDTAVYYCAKTTVSDPYYYGMDVWGQGTTV |
| | TVSSGGSEGKSSGSGSESKSTGGSSYELTQPPSVSVAPGQTARITCGGNNIGSKSVHWY |
| | QQKPGQAPVLVVYDDSDRPSGIPERFSGTNSGNTATLTISRAEAGDEADYYCQVWDSSS |
| | DHVVFGGGTKLTVL |
| | (SEQ ID NO: 1526) |
| PSMA_P72_A10 | SYELMQPPSVSVSPGQTARITCSGDALPKQYAYWYQQKPGQAPVLVIYKDSERPSGIPV |
| scFv LH | RFSGSSSGTTVTLTITGVQAEDEADYYCQSADSSGTYVFGTGTKVTVLGGSEGKSSGSG |
| | SESKSTGGSQVQLVESGGGVVQPGRSLRLSCAASGFTFSSYNMNWVRQAPGKGLEWVAI |
| | IYYDGSNKYYADSVKGRFTISRDISKNTLYLQMNSLRAEDTAVYYCARERGRDYYGMDV |
| | WGQGTTVTVSS |
| | (SEQ ID NO: 1527) |
| PSMA_P72_C01 | QSVLTQPPSVSVAPGQTARITCGGNNSGSKSVHWYQQKPGQAPVLVVYDDSDRPSGIPE |
| scFv LH | RFSGSNSGNTATLTISRVEAGDEADYYCQVWDSSSDHGVFGGGTKLTVLGGSEGKSSGS |
| | GSESKSTGGSQVQLVESGGGEVQPGRSLRLSCAASGFSFSGYGMHWVRQAPGKGLEWVA |
| | VMSYDGSNRFYVDSVRGRFSISRDNSKNTLYLQMNSLRPEDTAVYYCARDTVWGSHPDA |
| | FDIWGQGTVVTVSS |
| | (SEQ ID NO: 1528) |
| PSMA_P72_D01 | QSVLTQPASVSGSPGQSITISCTGTSSDVGGYNYVSWYQQHPGKAPKLMIYEVSNRPSG |
| scFv LH | VSNRFSGSKSGNTASLTISGLQAEDEADYYCSSYTSSYTYVFGTGTKLTVLGGSEGKSS |
| | GSGSESKSTGGSEVQLVESGGDLVQPGGSLRLSCAASGFTENNYNMNWVRQAPGKGLEW |
| | VSHISTSSSNKYYADSVKGRESISRDIAKNSMYLQMNSLRDEDTAVYYCARDGVGADYG |
| | DYYYYGMDVWGQGTTVTVSS |
| | (SEQ ID NO: 1529) |
| PSMA_P72_E07 | QSVLTQPPSASGTPGQGVTISCSGSSSNIGSNTVNWFQQLPGTAPKLLIYSDNQRPSGV |
| scFv LH | PDRFSGSKSGTSASLAISGLQSEDEADYYCAAWDDSLNGYVFGTGTKVTVLGGSEGKSS |
| | GSGSESKSTGGSEVQLVESGGGVVQPGRSLRLSCAASGFTFITYGMHWVRQAPGKGLEW |
| | VAVVSFDESNKYYADSVKGRFTISRDNSKNTLYLQMNSLRAEDTAVYYCARALRDGNNW |
| | DYFNGMDVWGQGTTVTVSS |
| | (SEQ ID NO: 1530) |
| PSMA_P72_A11 | QVQLQESGGDVVQPGRSLRLSCAASGESFSGYGLHWVRQAPGRGLEWVTLISYDGSNKY |
| scFv HL | YADSVKGRFTISRDNSKNTLYLQMNSLRAEDTAVYYCAKTTVSDPYYYGMDVWGQGTTV |
| | TVSSGGSEGKSSGSGSESKSTGGSSYELTQPPSVSVAPGQTARITCGGNNIGSKSVHWY |
| | QQKPGQAPVLVVYDDSDRPSGIPERFSGTNSGNTATLTISRAEAGDEADYYCQVWDSSS |
| | DHVVFGGGTKLTVL |
| | (SEQ ID NO: 1531) |

Exemplary PSMA cDNAs sequences are provided below.

(PSMB889 and PSMB890 VH cDNA)
SEQ ID NO: 1026
GAGGTGCAACTGGTCGAATCTGGAGGGGGACTGGTTAAGCCTGGGGGTAGCTTGCGTCTTAGCTGTG
CCGCTAGCGGCTTCACCTTCTCACGTTACAATATGAATTGGGTTCGACAGGCCCCTGGCAAAGGTCT
TGAGTGGGTTTCATCCATTAACTCAAATTCACGATATATCTACTACGCAGATTCTGTTAAGGGTCGA
TTCACTATATCTCGAGACAGTGCCAAGAATTCCTTGTACTTGCAGATGAATAGCTTGAGGGCCGAAG
ATACTGCCGTGTACTACTGTGCAAAGACAATGGGAGATTATTATTACTATTACGGCATGGACGTGTG
GGGGCAGGGGACAACAGTCACCGTAAGCAGT
(PSMB891 and PSMB892 VH cDNA)
SEQ ID NO: 1028
GAGGTGCAATTGGTTGAGAGTGGCGGGGGGGTCGTTCAACCAGGTCGCAGCTTGCGACTCTCTTGTG
CCGCCAGCGGTTTCACATTCATAACTTACGGGATGCACTGGGTGCGACAGGCACCCGGAAAGGGACT
GGAGTGGGTTGCTGTGGTGTCTTTCGATGAGTCTAATAAGTACTATGCAGACTCTGTTAAAGGCCGA
TTTACTATTAGCCGAGACAACTCTAAAAATACTCTGTACCTCCAGATGAATAGTTTGCGAGCAGAAG
ATACAGCAGTTTATTATTGTGCCAGGGCTCTTAGGGACGGTAATAATTGGGATTACTTTAACGGCAT
GGACGTATGGGGGCAAGGCACTACCGTTACTGTATCATCC
(PSMB893 and PSMB894 VH cDNA)
SEQ ID NO: 1030
CAAGTGCAGTTGGTTGAGTCAGGTGGAGGAGTGGTCCAGCCTGGTCGGAGCCTCAGATTGTCCTGCG
TGGCAAGCGGATTCACCTTTTCTTCTTACGGTATTCACTGGGTACGGCAAGCACCAGGGAAAGGGTT
GGAGTGGGTCGCTGTGATTTGGTACGACGGGTCAAATAAGTACTACGCCGATTCCGTGAAGGGTCGC
TTTACAATCTCTAGGGATAACTCAAAAAACACACTGTACCTGCAAATGAATAGTCTCCGTGCAGAAG
ACACAGCAGTATATTATTCTGTGCGCGGAGTAGGGCCCACTTCTTATTATTACAACTACGGTATGGA
TGTATGGGGCCAGGGCACTACCGTTACTGTATCTTCC
(PSMB895, PSMB946, PSMB896 and PSMB947 VH cDNA)
SEQ ID NO: 1032
GAGGTACAACTTGTGGAAAGTGGAGGCGGTCTTGTCCAACCTGGAGGATCTCTCCGATTGAGTTGCG
CAGCCAGCGGGTTTACTTTTTCTTCATACGCCATGTCCTGGGTGCGGCAAGCACCAGGTAAAGGACT
TGAGTGGGTATCTGCTATTTCAGGGGGGATAGGCTCAACATACTATGCTGATAGCGTGAAAGGTAGG
TTCACCATTTCCCGTGACAATAGTAAAAACACATTGTGGCTCCAAATGAACAGCCTTAGGGCTGAAG
ACACCGCTGTTTACTACTGCGCTAAAGACGGTGTAGGGGCAACTCCCTATTACTTCGATTATTGGGG
ACAAGGAACCTTGGTAACAGTTTCAAGC
(PSMB897, PSMB948, PSMB898 and PSMB949 VH cDNA)
SEQ ID NO: 1034
GAAGTCCAGTTGGTAGAATCTGGAGGCGGGCTGGTACAGCCTGGCGGTTCCTTGCGCCTCTCATGTG
CCGCAAGCGGGTTTACCTTCAGCTCTTACGCAATGTCATGGGTGCGTCAGGCCCCTGGAAAAGGTCT
CGAGTGGGTCAGTGCCATTTCTGGGGGCTCCGGCTCCACCTACTACGCAGATTCAGTTAAAGGGAGA
TTTACAATCTCAAGAGATAACAGTAAAAACACCCTCTACCTCCAGATGAACTCACTTCGAGCTGAGG
ATTCAGCAGTATATTACTGTGCTAAAGACGGTGTAGGTGCAACTCCCTACTATTTCGACTATTGGGG
CCAAGGGACTTTGGTGACAGTAAGTAGT
(PSMB899 VH cDNA)
SEQ ID NO: 1036
GAGGTGCAGCTTGTAGAATCAGGGGGAGGACTGGTGCAGCCAGGCGGATCATTGAGACTGAGTTGTA
CTGCCAGCGGATTCATATTCTCCAGTTATGCCATGAGCTGGGTGCGGCAGGCACCTGGAAAAGGGCT
TGAGTGGGTCTCTGCCATAAGCGGTGGATACGGTGCTCCATACTATGCTGACACCGTAAAAGGGCGG
TTCACCATCTCCCGAGACAATAGTAAGAACACCCTGTATCTCCAGATGAACTCCCTCCGCGCTGAAG
ATACAGCCGTTTATTACTGCGCCAAAGATGGAGTCGGGGCCACTCCATACTACTTCGATGATTGGGG
ACAAGGGATCCTCGTCACTGTTAGCTCA
(PSMB896-G100A VH cDNA)
SEQ ID NO: 1532
GAGGTGCAGCTGGTTGAATCTGGCGGAGGATTGGTTCAGCCTGGCGGTTCTCTGAGACTGTCTTGTG
CCGCTTCCGGCTTCACCTTCTCCAGCTACGCTATGTCCTGGGTCCGACAGGCTCCTGGCAAAGGACT
GGAATGGGTGTCCGCTATCTCTGGCGGCATCGGCTCTACCTACTACGCCGACTCTGTGAAGGGCAGA
TTCACCATCAGCCGGGACAACTCCAAGAACACCCTGTGGCTGCAGATGAACTCCCTGAGAGCCGAGG
ATACCGCCGTGTACTACTGTGCCAAGGATGCCGTGGGCGCTACCCCTTACTACTTCGATTATTGGGG
CCAGGGCACCCTGGTCACCGTTTCTTCT
(PSMB889 VL cDNA)
SEQ ID NO: 1037
CAATCCGTGCTTACACAACCACCATCAGTAAGCGGGGCTCCAGGCCAAAGAGTAACCATCTCATGCA
CTGGTTCCAGCTTCAACCTTGGTGCCGGATATGATGTCCATTGGTATCAACAAGTCCCTGGAACCGT
CCCAAAACTCCTGATCTACGATAATTCAAATCGACCTTCCGGAGTGCCAGATCGGTTCAGTGGATCT
AAAAGCGGCACCAGTGCCTCTCTTGCCATTACTGGATTGCAAGCTGAAGATGAGACAGTTTATTACT
GTCAGTCCTACGATTCTAGTTTGTCCGGAGTGGTTTTCGGTGGTGGTACTAAGCTCACCGTGCTG
(PSMB890 VL cDNA)
SEQ ID NO: 1038
AGCAGCGAGCTGACACAACCTCCCTCCGTTTCTGGAGCCCCAGGTCAAAGAGTGACTATTAGTTGTG
CAGGCTCCCTTTCAAATATAGGAGCAGGTTATGATGTTCATTGGTATCAACAACTTCCAGGCACAGC
ACCAAAACTCCTTATATACGGAAACATAAATCGCTTGAGCGGTGTACCCGAACGCTTCAGTGGCAGC
AAGTCTGGCACCAGCGCTTCCTTGGCAATAACCGGACTCCAGGCTGAGGATGGCGCTGATTACTATT
GCCAGTCCTATGATAGCTCCCTCTCTTCATATGTCTTCGGAACTGGTACAAAAGTGACCGTGCTT
(PSMB891 and PSMB893 VL cDNA)
SEQ ID NO: 1039
CAGTCCGTGTTGACACAACCTCCAAGCGCCTCAGGGACCCCAGGACAGGGTGTGACAATATCCTGTT
CCGGCAGTAGCAGTAACATTGGGTCTAATACCGTTAACTGGTTCCAGCAACTGCCCGGCACAGCTCC
CAAACTTTTGATCTACAGTGACAACCAACGACCCTCTGGCGTACCCGACCGTTTCTCAGGTTCCAAA
AGCGGGACCTCAGCATCTCTCGCTATCTCCGGTCTGCAAAGTGAGGACGAAGCCGATTACTACTGTG
CAGCCTGGGACGATTCACTGAATGGGTATGTTTTCGGAACTGGTACCAAAGTGACTGTGTTG
(PSMB892 and PSMB894 VL cDNA)
SEQ ID NO: 1040
CAGTCCGTTCTCACACAGCCACCATCAGTAAGTGGCGCTCCCGGTCAAAGAGTAACTATTTCATGTA
CTGGTAGTTCAAGCAATATAGGTGCTGACTATGACGTCCATTGGTATCAGCACTTGCCCGGCACTGC
ACCTAAACTTCTGATATACGGAAATAGTAACAGACCTAGTGGGGTTCCAGACCGTTTCTCCGGGAGT
AAAAGTGGAACAAGCGCTTCACTTGCAATTACTGGCCTCCAGGCTGAAGATGAAACCGACTATTACT
GCCAAAGCTACGATTCTAGCCTGTCTGGGTGGGTTTTCGGCGGGGGAACTAAATTGACCGTCTTG
(PSMB895, PSMB946, PSMB897 and PSMB948 VL CDNA)
SEQ ID NO: 1043
CAATCTGTCCTGACTCAACCTCCCTCAGTCTCAGCCGCACCAGGACAGAAGGTGACAATTAGCTGTT
CAGGTTCTTCAAGTAACATCGGTAACAACTACGTCTCATGGTATCAACAGCTTCCCGGAACAGCACC
CAAACTGCTGATATATGATAACAACAAACGGCCATCTGGAATACCAGACCGGTTCTCAGGCTCCAAG
AGCGGTACTAGCGCAACTTTGGGAATCACCGGTTTGCAGACTGGGGATGAGGCAGACTATTACTGCG
GCACCTGGGATTCCAGTCTGTCTGCTTATGTTTTTGGGACCGGGACAAAGGTGACTGTCCTT
(PSMB896, PSMB947, PSMB898 and PSMB949 VL cDNA)
SEQ ID NO: 1044
CAGAGTGTCCTTACTCAGCCTCCTAGCGTTAGCGCCGCCCCTGGACAGAAGGTTACTATCTCCTGCT
CAGGGAGTTCCAGTAATATTGGAATCAATTATGTGAGTTGGTATCAGCAGTTGCCCGGCACCGCTCC
TAAATTGCTTATCTATGACAACAATAAACGACCTAGTGGTATCCCTGATCGTTTTTCTGGATCAAAA
TCTGGTACTAGCGCAACCCTCGGTATCACCGGACTGCAAACAGGTGATGAAGCAGACTATTATTGCG
GCACCTGGGACTCATCACTCTCCGCCGTCGTTTTCGGGGGCGGAACCAAACTTACAGTATTG
(PSMB899 VL cDNA)
SEQ ID NO: 1047
CAAAGCGTGCTGACACAGCCTCCATCAGTCAGCGCAGCCCCAGGACAGAAAGTCACTATTTCTTGCA
GCGGTAGCAGCTCTAATATCGGCAATAATTACGTCTCCTGGTATCAGCAGCTCCCCGGCACTGCACC
TAAGTTGCTCATCTTCGATAATAACAAACGTCCTTCAGGCATTCCCGACCGATTCTCAGGTTCAAAA
TCAGGAACCAGTGCTACTTTGGGAATAACAGGTCTGCAAACTGGTGATGAAGCTGATTACTACTGCG
GTACCTGGGATAGCTCTCTCAGTGCCTATGTATTCGGGACTGGCACAAAGGTTACTGTTCTG
(PSMB896-G100A VL cDNA)
SEQ ID NO: 1533
CAGTCTGTGCTGACCCAGCCTCCTTCTGTGTCTGCTGCTCCTGGCCAGAAAGTGACCATCTCCTGCT
CCGGCTCCTCCTCCAACATCGGCATCAACTACGTGTCCTGGTATCAGCAGCTGCCCGGCACAGCTCC
CAAACTGCTGATCTACGACAACAACAAGCGGCCCAGCGGCATCCCTGACAGATTCTCCGGATCTAAG
TCCGGCACCTCTGCTACCCTGGGCATCACAGGATTGCAGACAGGCGACGAGGCCGACTACTATTGCG
GCACCTGGGACTCTTCTCTGTCCGCCGTTGTTTTTGGCGGTGGCACCAAGCTGACAGTGCTG
(PSMB889 and PSMB890 HC cDNA)
SEQ ID NO: 1048
GAGGTGCAACTGGTCGAATCTGGAGGGGGACTGGTTAAGCCTGGGGGTAGCTTGCGTCTTAGCTGTG
CCGCTAGCGGCTTCACCTTCTCACGTTACAATATGAATTGGGTTCGACAGGCCCCTGGCAAAGGTCT
TGAGTGGGTTTCATCCATTAACTCAAATTCACGATATATCTACTACGCAGATTCTGTTAAGGGTCGA
TTCACTATATCTCGAGACAGTGCCAAGAATTCCTTGTACTTGCAGATGAATAGCTTGAGGGCCGAAG
ATACTGCCGTGTACTACTGTGCAAAGACAATGGGAGATTATTATTACTATTACGGCATGGACGTGTG
GGGGCAGGGGACAACAGTCACCGTAAGCAGTGCTTCCACCAAGGGCCCATCCGTCTTCCCCCTGGCA
CCCTCCTCCAAGAGCACCTCTGGGGGCACAGCGGCCCTGGGCTGCCTGGTCAAGGACTACTTCCCCG
AACCGGTGACGGTGTCGTGGAACTCAGGCGCCCTGACCAGCGGCGTGCACACCTTCCCGGCTGTCCT
ACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGTTGAGCCCAAAT
CTTGTGACAAAACTCACACATGTCCACCGTGCCCAGCACCTGAAGCAGCAGGGGGACCGTCAGTCTT
CCTCTTCCCCCCAAAACCCAAGGACACCCTCATGATCTCCCGGACCCCTGAGGTCACATGCGTGGTG
GTGAGCGTGAGCCACGAAGACCCTGAGGTCAAGTTCAACTGGTACGTGGACGGCGTGGAGGTGCATA
ATGCCAAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCAACAAAGCCCTCCCAGCC
CCCATCGAGAAAACCATCTCCAAAGCCAAAGGGCAGCCCCGAGAACCACAGGTGTACACCCTGCCCC
CATCCCGGGAGGAGATGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCGTG
CTGGACTCCGACGGCTCCTTCTTCCTCTACAGCCGGCTCACCGTGGACAAGTCTAGATGGCAGCAGG
GGAACGTCTTCTCATGCTCCGTGATGCATGAGGCTCTGCACAACCACTACACGCAGAAGAGCCTCTC
CCTGTCTCCGGGT
(PSMB891 and PSMB892 HC cDNA)
SEQ ID NO: 1050
GAGGTGCAATTGGTTGAGAGTGGCGGGGGGGTCGTTCAACCAGGTCGCAGCTTGCGACTCTCTTGTG
CCGCCAGCGGTTTCACATTCATAACTTACGGGATGCACTGGGTGCGACAGGCACCCGGAAAGGGACT
GGAGTGGGTTGCTGTGGTGTCTTTCGATGAGTCTAATAAGTACTATGCAGACTCTGTTAAAGGCCGA
TTTACTATTAGCCGAGACAACTCTAAAAATACTCTGTACCTCCAGATGAATAGTTTGCGAGCAGAAG
ATACAGCAGTTTATTATTGTGCCAGGGCTCTTAGGGACGGTAATAATTGGGATTACTTTAACGGCAT
GGACGTATGGGGGCAAGGCACTACCGTTACTGTATCATCCGCTTCCACCAAGGGCCCATCCGTCTTC
CCCCTGGCACCCTCCTCCAAGAGCACCTCTGGGGGCACAGCGGCCCTGGGCTGCCTGGTCAAGGACT
ACTTCCCCGAACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAAAGTTG
AGCCCAAATCTTGTGACAAAACTCACACATGTCCACCGTGCCCAGCACCTGAAGCAGCAGGGGGACC
GTCAGTCTTCCTCTTCCCCCCAAAACCCAAGGACACCCTCATGATCTCCCGGACCCCTGAGGTCACA
TGCGTGGTGGTGAGCGTGAGCCACGAAGACCCTGAGGTCAAGTTCAACTGGTACGTGGACGGCGTGG
AGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACCACAGGTGTACA
CCCTGCCCCCATCCCGGGAGGAGATGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCCGGCTCACCGTGGACAAGTCTAGAT
GGCAGCAGGGGAACGTCTTCTCATGCTCCGTGATGCATGAGGCTCTGCACAACCACTACACGCAGAA
GAGCCTCTCCCTGTCTCCGGGT
(PSMB893 and PSMB894 HC cDNA)
SEQ ID NO: 1052
CAAGTGCAGTTGGTTGAGTCAGGTGGAGGAGTGGTCCAGCCTGGTCGGAGCCTCAGATTGTCCTGCG
TGGCAAGCGGATTCACCTTTTCTTCTTACGGTATTCACTGGGTACGGCAAGCACCAGGGAAAGGGTT
GGAGTGGGTCGCTGTGATTTGGTACGACGGGTCAAATAAGTACTACGCCGATTCCGTGAAGGGTCGC
TTTACAATCTCTAGGGATAACTCAAAAAACACACTGTACCTGCAAATGAATAGTCTCCGTGCAGAAG
ACACAGCAGTATATTATTCTGTGCGCGGAGTAGGGCCCACTTCTTATTATTACAACTACGGTATGGA
TGTATGGGGCCAGGGCACTACCGTTACTGTATCTTCCGCTTCCACCAAGGGCCCATCCGTCTTCCCC
CTGGCACCCTCCTCCAAGAGCACCTCTGGGGGCACAGCGGCCCTGGGCTGCCTGGTCAAGGACTACT
TCCCCGAACCGGTGACGGTGTCGTGGAACTCAGGCGCCCTGACCAGCGGCGTGCACACCTTCCCGGC
TGTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAAAGTTGAGC
CCAAATCTTGTGACAAAACTCACACATGTCCACCGTGCCCAGCACCTGAAGCAGCAGGGGGACCGTC
AGTCTTCCTCTTCCCCCCAAAACCCAAGGACACCCTCATGATCTCCCGGACCCCTGAGGTCACATGC
GTGGTGGTGAGCGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCAAGGTCTCCAACAAAGCCCTC
CCAGCCCCCATCGAGAAAACCATCTCCAAAGCCAAAGGGCAGCCCCGAGAACCACAGGTGTACACCC
TGCCCCCATCCCGGGAGGAGATGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCT
CCCGTGCTGGACTCCGACGGCTCCTTCTTCCTCTACAGCCGGCTCACCGTGGACAAGTCTAGATGGC
AGCAGGGGAACGTCTTCTCATGCTCCGTGATGCATGAGGCTCTGCACAACCACTACACGCAGAAGAG
CCTCTCCCTGTCTCCGGGT
(PSMB895 and PSMB896 HC cDNA)
SEQ ID NO: 1054
GAGGTACAACTTGTGGAAAGTGGAGGCGGTCTTGTCCAACCTGGAGGATCTCTCCGATTGAGTTGCG
CAGCCAGCGGGTTTACTTTTTCTTCATACGCCATGTCCTGGGTGCGGCAAGCACCAGGTAAAGGACT
TGAGTGGGTATCTGCTATTTCAGGGGGGATAGGCTCAACATACTATGCTGATAGCGTGAAAGGTAGG
TTCACCATTTCCCGTGACAATAGTAAAAACACATTGTGGCTCCAAATGAACAGCCTTAGGGCTGAAG
ACACCGCTGTTTACTACTGCGCTAAAGACGGTGTAGGGGCAACTCCCTATTACTTCGATTATTGGGG
ACAAGGAACCTTGGTAACAGTTTCAAGCGCTTCCACCAAGGGCCCATCCGTCTTCCCCCTGGCACCC
TCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCTT
GTGACAAAACTCACACATGTCCACCGTGCCCAGCACCTGAAGCAGCAGGGGGACCGTCAGTCTTCCT
CTTCCCCCCAAAACCCAAGGACACCCTCATGATCTCCCGGACCCCTGAGGTCACATGCGTGGTGGTG
AGCGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCTACAGCCGGCTCACCGTGGACAAGTCTAGATGGCAGCAGGGGA
ACGTCTTCTCATGCTCCGTGATGCATGAGGCTCTGCACAACCACTACACGCAGAAGAGCCTCTCCCT
GTCTCCGGGT
(PSMB896-G100A HC cDNA)
SEQ ID NO: 1501
GAGGTGCAGCTGGTTGAATCTGGCGGAGGATTGGTTCAGCCTGGCGGTTCTCTGAGACTGTCTTGTG
CCGCTTCCGGCTTCACCTTCTCCAGCTACGCTATGTCCTGGGTCCGACAGGCTCCTGGCAAAGGACT
GGAATGGGTGTCCGCTATCTCTGGCGGCATCGGCTCTACCTACTACGCCGACTCTGTGAAGGGCAGA
TTCACCATCAGCCGGGACAACTCCAAGAACACCCTGTGGCTGCAGATGAACTCCCTGAGAGCCGAGG
ATACCGCCGTGTACTACTGTGCCAAGGATGCCGTGGGCGCTACCCCTTACTACTTCGATTATTGGGG
CCAGGGCACCCTGGTCACCGTTTCTTCTGCCTCCACCAAGGGCCCATCGGTCTTCCCCCTGGCACCC
TCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCTT
GTGACAAAACTCACACATGCCCACCGTGCCCAGCACCTGAAGCCGCCGGGGGACCGTCAGTCTTCCT
CTTCCCCCCAAAACCCAAGGACACCCTCATGATCTCCCGGACCCCTGAGGTCACATGCGTGGTGGTG
AGCGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTGTCGAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGTCCTGCGCCGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCGTGAGCAAGCTCACCGTGGACAAGAGCAGATGGCAGCAGGGGA
ACGTCTTCTCATGCTCCGTGATGCATGAGGCTCTGCACAACCGGTTCACGCAGAAGTCTCTCTCCCT
GTCTCCGGGAAAA
(PSMB897 and PSMB898 HC cDNA)
SEQ ID NO: 1056
GAAGTCCAGTTGGTAGAATCTGGAGGCGGGCTGGTACAGCCTGGCGGTTCCTTGCGCCTCTCATGTG
CCGCAAGCGGGTTTACCTTCAGCTCTTACGCAATGTCATGGGTGCGTCAGGCCCCTGGAAAAGGTCT
CGAGTGGGTCAGTGCCATTTCTGGGGGCTCCGGCTCCACCTACTACGCAGATTCAGTTAAAGGGAGA
TTTACAATCTCAAGAGATAACAGTAAAAACACCCTCTACCTCCAGATGAACTCACTTCGAGCTGAGG
ATTCAGCAGTATATTACTGTGCTAAAGACGGTGTAGGTGCAACTCCCTACTATTTCGACTATTGGGG
CCAAGGGACTTTGGTGACAGTAAGTAGTGCTTCCACCAAGGGCCCATCCGTCTTCCCCCTGGCACCC
TCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCTT
GTGACAAAACTCACACATGTCCACCGTGCCCAGCACCTGAAGCAGCAGGGGGACCGTCAGTCTTCCT
CTTCCCCCCAAAACCCAAGGACACCCTCATGATCTCCCGGACCCCTGAGGTCACATGCGTGGTGGTG
AGCGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCTACAGCCGGCTCACCGTGGACAAGTCTAGATGGCAGCAGGGGA
ACGTCTTCTCATGCTCCGTGATGCATGAGGCTCTGCACAACCACTACACGCAGAAGAGCCTCTCCCT
GTCTCCGGGT
(PSMB899 HC cDNA)
SEQ ID NO: 1058
GAGGTGCAGCTTGTAGAATCAGGGGGAGGACTGGTGCAGCCAGGCGGATCATTGAGACTGAGTTGTA
CTGCCAGCGGATTCATATTCTCCAGTTATGCCATGAGCTGGGTGCGGCAGGCACCTGGAAAAGGGCT
TGAGTGGGTCTCTGCCATAAGCGGTGGATACGGTGCTCCATACTATGCTGACACCGTAAAAGGGCGG
TTCACCATCTCCCGAGACAATAGTAAGAACACCCTGTATCTCCAGATGAACTCCCTCCGCGCTGAAG
ATACAGCCGTTTATTACTGCGCCAAAGATGGAGTCGGGGCCACTCCATACTACTTCGATGATTGGGG
ACAAGGGATCCTCGTCACTGTTAGCTCAGCTTCCACCAAGGGCCCATCCGTCTTCCCCCTGGCACCC
TCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCTT
GTGACAAAACTCACACATGTCCACCGTGCCCAGCACCTGAAGCAGCAGGGGGACCGTCAGTCTTCCT
CTTCCCCCCAAAACCCAAGGACACCCTCATGATCTCCCGGACCCCTGAGGTCACATGCGTGGTGGTG
AGCGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCTACAGCCGGCTCACCGTGGACAAGTCTAGATGGCAGCAGGGGA
ACGTCTTCTCATGCTCCGTGATGCATGAGGCTCTGCACAACCACTACACGCAGAAGAGCCTCTCCCT
GTCTCCGGGT
(PSMB946 and PSMB947 HC cDNA)
SEQ ID NO: 1059
GAGGTGCAGCTGGTTGAATCTGGCGGAGGATTGGTTCAGCCTGGCGGTTCTCTGAGACTGTCTTGTG
CCGCTTCCGGCTTCACCTTCTCCAGCTACGCTATGTCCTGGGTCCGACAGGCTCCTGGCAAAGGACT
GGAATGGGTGTCCGCTATCTCTGGCGGCATCGGCTCTACCTACTACGCCGACTCTGTGAAGGGCAGA
TTCACCATCAGCCGGGACAACTCCAAGAACACCCTGTGGCTGCAGATGAACTCCCTGAGAGCCGAGG
ATACCGCCGTGTACTACTGTGCCAAAGATGGCGTGGGCGCTACCCCTTACTACTTCGATTATTGGGG
CCAGGGCACCCTGGTCACCGTTTCTTCTGCCTCCACCAAGGGTCCATCGGTCTTCCCCCTGGCACCC
TCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCTT
GTGACAAAACTCACACTTGTCCACCGTGCCCAGCACCTGAAGCAGCAGGGGGACCGTCAGTCTTCCT
CTTCCCCCCAAAACCCAAGGACACCCTCATGATCTCCCGGACCCCTGAGGTCACATGCGTGGTGGTG
AGCGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTGTCCAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCTACAGCCGGCTCACCGTGGACAAGTCCAGATGGCAGCAGGGGA
ACGTCTTCTCATGCTCCGTGATGCATGAGGCTCTGCACAACCACTACACGCAGAAGTCTCTCTCCCT
GTCTCCGGGAAAA
(PSMB948 and PSMB949 HC cDNA)
SEQ ID NO: 1061
GAGGTGCAGCTGGTTGAATCTGGCGGAGGATTGGTTCAGCCTGGCGGTTCTCTGAGACTGTCTTGTG
CCGCTTCCGGCTTCACCTTCTCCAGCTACGCTATGTCCTGGGTCCGACAGGCTCCTGGCAAAGGACT
GGAATGGGTGTCCGCTATCTCTGGCGGATCCGGCTCTACCTACTACGCCGACTCTGTGAAGGGCAGA
TTCACCATCAGCCGGGACAACTCCAAGAACACCCTGTACCTGCAGATGAACTCCCTGAGAGCCGAGG
ACTCCGCCGTGTACTACTGTGCTAAAGATGGCGTGGGCGCTACCCCTTACTACTTCGATTATTGGGG
CCAGGGCACCCTGGTCACCGTTTCTTCTGCCTCCACCAAGGGTCCATCGGTCTTCCCCCTGGCACCC
TCCTCCAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGAGCCCAAATCTT
GTGACAAAACTCACACTTGTCCACCGTGCCCAGCACCTGAAGCAGCAGGGGGACCGTCAGTCTTCCT
CTTCCCCCCAAAACCCAAGGACACCCTCATGATCTCCCGGACCCCTGAGGTCACATGCGTGGTGGTG
AGCGTGAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACCGTCCT
GCACCAGGACTGGCTGAATGGCAAGGAGTACAAGTGCAAGGTGTCCAACAAAGCCCTCCCAGCCCCC
ATCGAGAAAACCATCTCCAAAGCCAAAGGGCAGCCCCGAGAACCACAGGTGTACACCCTGCCCCCAT
CCCGGGAGGAGATGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTG
GACTCCGACGGCTCCTTCTTCCTCTACAGCCGGCTCACCGTGGACAAGTCCAGATGGCAGCAGGGGA
ACGTCTTCTCATGCTCCGTGATGCATGAGGCTCTGCACAACCACTACACGCAGAAGTCTCTCTCCCT
GTCTCCGGGAAAA
(PSMB889 LC cDNA)
SEQ ID NO: 1063
CAATCCGTGCTTACACAACCACCATCAGTAAGCGGGGCTCCAGGCCAAAGAGTAACCATCTCATGCA
CTGGTTCCAGCTTCAACCTTGGTGCCGGATATGATGTCCATTGGTATCAACAAGTCCCTGGAACCGT
CCCAAAACTCCTGATCTACGATAATTCAAATCGACCTTCCGGAGTGCCAGATCGGTTCAGTGGATCT
AAAAGCGGCACCAGTGCCTCTCTTGCCATTACTGGATTGCAAGCTGAAGATGAGACAGTTTATTACT
GTCAGTCCTACGATTCTAGTTTGTCCGGAGTGGTTTTCGGTGGTGGTACTAAGCTCACCGTGCTGGG
TCAGCCCAAGGCTGCACCCAGTGTCACTCTGTTCCCGCCCTCCTCTGAGGAGCTTCAAGCCAACAAG
GCCACACTGGTGTGTCTCATAAGTGACTTCTACCCGGGAGCCGTGACAGTGGCCTGGAAGGCCGATA
GCAGCCCCGTCAAGGCGGGAGTGGAGACCACCACACCCTCCAAACAAAGCAACAACAAGTACGCGGC
CAGCAGCTATCTGAGCCTGACGCCTGAGCAGTGGAAGTCCCACAGAAGCTACAGCTGCCAGGTCACG
CATGAAGGGAGCACCGTGGAGAAGACAGTGGCCCCTACAGAATGTTCA
(PSMB890 LC cDNA)
SEQ ID NO: 1064
AGCAGCGAGCTGACACAACCTCCCTCCGTTTCTGGAGCCCCAGGTCAAAGAGTGACTATTAGTTGTG
CAGGCTCCCTTTCAAATATAGGAGCAGGTTATGATGTTCATTGGTATCAACAACTTCCAGGCACAGC
ACCAAAACTCCTTATATACGGAAACATAAATCGCTTGAGCGGTGTACCCGAACGCTTCAGTGGCAGC
AAGTCTGGCACCAGCGCTTCCTTGGCAATAACCGGACTCCAGGCTGAGGATGGCGCTGATTACTATT
GCCAGTCCTATGATAGCTCCCTCTCTTCATATGTCTTCGGAACTGGTACAAAAGTGACCGTGCTTGG
TCAGCCCAAGGCTGCACCCAGTGTCACTCTGTTCCCGCCCTCCTCTGAGGAGCTTCAAGCCAACAAG
GCCACACTGGTGTGTCTCATAAGTGACTTCTACCCGGGAGCCGTGACAGTGGCCTGGAAGGCCGATA
GCAGCCCCGTCAAGGCGGGAGTGGAGACCACCACACCCTCCAAACAAAGCAACAACAAGTACGCGGC
CAGCAGCTATCTGAGCCTGACGCCTGAGCAGTGGAAGTCCCACAGAAGCTACAGCTGCCAGGTCACG
CATGAAGGGAGCACCGTGGAGAAGACAGTGGCCCCTACAGAATGTTCA
(PSMB891 and PSMB893 LC cDNA)
SEQ ID NO: 1065
CAGTCCGTGTTGACACAACCTCCAAGCGCCTCAGGGACCCCAGGACAGGGTGTGACAATATCCTGTT
CCGGCAGTAGCAGTAACATTGGGTCTAATACCGTTAACTGGTTCCAGCAACTGCCCGGCACAGCTCC
CAAACTTTTGATCTACAGTGACAACCAACGACCCTCTGGCGTACCCGACCGTTTCTCAGGTTCCAAA
AGCGGGACCTCAGCATCTCTCGCTATCTCCGGTCTGCAAAGTGAGGACGAAGCCGATTACTACTGTG
CAGCCTGGGACGATTCACTGAATGGGTATGTTTTCGGAACTGGTACCAAAGTGACTGTGTTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
(PSMB892 and PSMB894 LC cDNA)
SEQ ID NO: 1066
CAGTCCGTTCTCACACAGCCACCATCAGTAAGTGGCGCTCCCGGTCAAAGAGTAACTATTTCATGTA
CTGGTAGTTCAAGCAATATAGGTGCTGACTATGACGTCCATTGGTATCAGCACTTGCCCGGCACTGC
ACCTAAACTTCTGATATACGGAAATAGTAACAGACCTAGTGGGGTTCCAGACCGTTTCTCCGGGAGT
AAAAGTGGAACAAGCGCTTCACTTGCAATTACTGGCCTCCAGGCTGAAGATGAAACCGACTATTACT
GCCAAAGCTACGATTCTAGCCTGTCTGGGTGGGTTTTCGGCGGGGGAACTAAATTGACCGTCTTGGG
TCAGCCCAAGGCTGCACCCAGTGTCACTCTGTTCCCGCCCTCCTCTGAGGAGCTTCAAGCCAACAAG
GCCACACTGGTGTGTCTCATAAGTGACTTCTACCCGGGAGCCGTGACAGTGGCCTGGAAGGCCGATA
GCAGCCCCGTCAAGGCGGGAGTGGAGACCACCACACCCTCCAAACAAAGCAACAACAAGTACGCGGC
CAGCAGCTATCTGAGCCTGACGCCTGAGCAGTGGAAGTCCCACAGAAGCTACAGCTGCCAGGTCACG
CATGAAGGGAGCACCGTGGAGAAGACAGTGGCCCCTACAGAATGTTCA
(PSMB895, PSMB846, PSMB897 and PSMB848 LC cDNA)
SEQ ID NO: 1069
CAATCTGTCCTGACTCAACCTCCCTCAGTCTCAGCCGCACCAGGACAGAAGGTGACAATTAGCTGTT
CAGGTTCTTCAAGTAACATCGGTAACAACTACGTCTCATGGTATCAACAGCTTCCCGGAACAGCACC
CAAACTGCTGATATATGATAACAACAAACGGCCATCTGGAATACCAGACCGGTTCTCAGGCTCCAAG
AGCGGTACTAGCGCAACTTTGGGAATCACCGGTTTGCAGACTGGGGATGAGGCAGACTATTACTGCG
GCACCTGGGATTCCAGTCTGTCTGCTTATGTTTTTGGGACCGGGACAAAGGTGACTGTCCTTGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
(PSMB896, PSMB847, PSMB898 and PSMB849 LC cDNA)
SEQ ID NO: 1070
CAGAGTGTCCTTACTCAGCCTCCTAGCGTTAGCGCCGCCCCTGGACAGAAGGTTACTATCTCCTGCT
CAGGGAGTTCCAGTAATATTGGAATCAATTATGTGAGTTGGTATCAGCAGTTGCCCGGCACCGCTCC
TAAATTGCTTATCTATGACAACAATAAACGACCTAGTGGTATCCCTGATCGTTTTTCTGGATCAAAA
TCTGGTACTAGCGCAACCCTCGGTATCACCGGACTGCAAACAGGTGATGAAGCAGACTATTATTGCG
GCACCTGGGACTCATCACTCTCCGCCGTCGTTTTCGGGGGCGGAACCAAACTTACAGTATTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
(PSMB899 LC cDNA)
SEQ ID NO: 1073
CAAAGCGTGCTGACACAGCCTCCATCAGTCAGCGCAGCCCCAGGACAGAAAGTCACTATTTCTTGCA
GCGGTAGCAGCTCTAATATCGGCAATAATTACGTCTCCTGGTATCAGCAGCTCCCCGGCACTGCACC
TAAGTTGCTCATCTTCGATAATAACAAACGTCCTTCAGGCATTCCCGACCGATTCTCAGGTTCAAAA
TCAGGAACCAGTGCTACTTTGGGAATAACAGGTCTGCAAACTGGTGATGAAGCTGATTACTACTGCG
GTACCTGGGATAGCTCTCTCAGTGCCTATGTATTCGGGACTGGCACAAAGGTTACTGTTCTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
(PSMB896-G100A LC cDNA)
SEQ ID NO: 1534
CAGTCTGTGCTGACCCAGCCTCCTTCTGTGTCTGCTGCTCCTGGCCAGAAAGTGACCATCTCCTGCT
CCGGCTCCTCCTCCAACATCGGCATCAACTACGTGTCCTGGTATCAGCAGCTGCCCGGCACAGCTCC
CAAACTGCTGATCTACGACAACAACAAGCGGCCCAGCGGCATCCCTGACAGATTCTCCGGATCTAAG
TCCGGCACCTCTGCTACCCTGGGCATCACAGGATTGCAGACAGGCGACGAGGCCGACTACTATTGCG
GCACCTGGGACTCTTCTCTGTCCGCCGTTGTTTTTGGCGGTGGCACCAAGCTGACAGTGCTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTCGAAACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA

Exemplary amino acid and nucleic acid sequence of HC and LC of certain PSMA antibodies in DuoBody format or in ScFv LH-hinge CH2-CH3 format are provided below.

TABLE 11
PSMA Antibodies (DuoBody)

| | HC | LC | HC | LC |
|---|---|---|---|---|
| | AA | AA | DNA | DNA |
| PSMB889 | SEQ ID NO: 33 | SEQ ID NO: 4 | SEQ ID NO: 1048 | SEQ ID NO:1063 |
| PSMB890 | SEQ ID NO: 33 | SEQ ID NO: 68 | SEQ ID NO: 1048 | SEQ ID NO: 1064 |
| PSMB891 | SEQ ID NO: 101 | SEQ ID NO: 102 | SEQ ID NO: 1050 | SEQ ID NO: 1065 |
| PSMB892 | SEQ ID NO: 101 | SEQ ID NO: 136 | SEQ ID NO: 1050 | SEQ ID NO: 1066 |
| PSMB893 | SEQ ID NO: 169 | SEQ ID NO: 102 | SEQ ID NO: 1052 | SEQ ID NO: 1065 |
| PSMB894 | SEQ ID NO: 169 | SEQ ID NO: 136 | SEQ ID NO: 1052 | SEQ ID NO: 1066 |
| PSMB895 | SEQ ID NO: 237 | SEQ ID NO: 238 | SEQ ID NO: 1054 | SEQ ID NO: 1069 |
| PSMB896 | SEQ ID NO: 237 | SEQ ID NO: 272 | SEQ ID NO: 1054 | SEQ ID NO: 1070 |
| PSMB896- | SEQ ID NO: 441 | SEQ ID NO: 272 | SEQ ID NO: 1501 | SEQ ID NO: 1534 |
| G100A | | | | |
| PSMB897 | SEQ ID NO: 305 | SEQ ID NO: 238 | SEQ ID NO: 1056 | SEQ ID NO: 1069 |
| PSMB898 | SEQ ID NO: 305 | SEQ ID NO: 272 | SEQ ID NO: 1056 | SEQ ID NO: 1070 |
| PSMB899 | SEQ ID NO: 373 | SEQ ID NO: 374 | SEQ ID NO: 1058 | SEQ ID NO: 1073 |

TABLE 12
PSMA Antibodies (ScFv_LH-hinge CH2-CH3)

| | ScFv_LH-hinge-CH2-CH3 | ScFv_LH-hinge CH2-CH3 |
|---|---|---|
| | AA | DNA |
| PSMB- | QSVLTQPPSVSAAPGQKVTISCSGSS | CAGTCTGTGCTGACCCAGCCTCCTTCTGTGTCTGCTGCTCCTGGCCAGAAAGTGACCATCTCCTGCTCCGGCT |
| 895 | SNIGNNYVSWYQQLPGTAPKLLIYDN | CCTCCTCCAACATCGGCAACAACTACGTGTCCTGGTATCAGCAGCTGCCCGGCACAGCTCCCAAACTGCTGAT |
| | NKRPSGIPDRESGSKSGTSATLGITG | CTACGACAACAACAAGCGGCCCAGCGGCATCCCTGACAGATTCTCCGGATCTAAGTCCGGCACCTCTGCTACC |
| | LQTGDEADYYCGTWDSSLSAYVFGTG | CTGGGCATCACAGGATTGCAGACAGGCGACGAGGCCGACTACTATTGCGGCACCTGGGACTCTTCCCTGTCCG |
| | TKVTVLGGSEGKSSGSGSESKSTGGS | CTTATGTGTTTGGCACCGGCACCAAAGTGACCGTGTTGGGCGGCTCCGAGGGCAAGAGCAGCGGCAGCGGCAG |
| | EVQLVESGGGLVQPGGSLRLSCAASG | CGAGAGCAAGAGCACCGGCGGCAGCGAGGTGCAGCTGGTTGAATCTGGCGGAGGATTGGTTCAGCCTGGCGGT |
| | FTFSSYAMSWVRQAPGKGLEWVSAIS | TCTCTGAGACTGTCTTGTGCCGCTTCCGGCTTCACCTTCTCCAGCTACGCTATGTCCTGGGTCCGACAGGCTC |
| | GGIGSTYYADSVKGRFTISRDNSKNT | CTGGCAAAGGACTGGAATGGGTGTCCGCTATCTCTGGCGGCATCGGCTCTACCTACTACGCCGACTCTGTGAA |
| | LWLQMNSLRAEDTAVYYCAKDGVGAT | GGGCAGATTCACCATCAGCCGGGACAACTCCAAGAACACCCTGTGGCTGCAGATGAACTCCCTGAGAGCCGAG |
| | PYYFDYWGQGTLVTVSSEPKSSDKTH | GATACCGCCGTGTACTACTGTGCCAAAGATGGCGTGGGCGCTACCCCTTACTACTTCGATTATTGGGGCCAGG |
| | TCPPCPAPEAAGGPSVFLFPPKPKDT | GCACCCTGGTCACCGTTTCTTCTGAGCCCAAATCTAGCGACAAAACTCACACTTGTCCACCGTGCCCAGCACC |
| | LMISRTPEVTCVVVSVSHEDPEVKEN | TGAAGCAGCAGGGGGACCGTCAGTCTTCCTCTTCCCCCCAAAACCCAAGGACACCCTCATGATCTCCCGGACC |
| | WYVDGVEVHNAKTKPREEQYNSTYRV | CCTGAGGTCACATGCGTGGTGGTGAGCGTGAGCCACGAAGACCCTGAGGTCAAGTTCAACTGGTACGTGGACG |
| | VSVLTVLHQDWLNGKEYKCKVSNKAL | GCGTGGAGGTGCATAATGCCAAGACAAAGCCGCGGGAGGAGCAGTACAACAGCACGTACCGTGTGGTCAGCGT |
| | PAPIEKTISKAKGQPREPQVYVLPPS | CCTCACCGTCCTGCACCAGGACTGGCTGAATGGCAAGGAGTACAAGTGCAAGGTGTCCAACAAAGCCCTCCCA |
| | REEMTKNQVSLLCLVKGFYPSDIAVE | GCCCCCATCGAGAAAACCATCTCCAAAGCCAAAGGGCAGCCCCGAGAACCACAGGTGTACGTGCTGCCCCCAT |
| | WESNGQPENNYLTWPPVLDSDGSFFL | CCCGGGAGGAGATGACCAAGAACCAGGTCAGCCTGCTGTGCCTGGTCAAAGGCTTCTATCCCAGCGACATCGC |
| | YSKLTVDKSRWQQGNVFSCSVMHEAL | CGTGGAGTGGGAGAGCAATGGGCAGCCGGAGAACAACTACCTCACCTGGCCTCCCGTGCTGGACTCCGACGGC |
| | HNHYTQKSLSLSPGK | TCCTTCTTCCTCTACAGCAAGCTCACCGTGGACAAGTCCAGATGGCAGCAGGGGAACGTCTTCTCATGCTCCG |
| | (SEQ ID NO: 1134) | TGATGCATGAGGCTCTGCACAACCACTACACGCAGAAGTCTCTCTCCCTGTCTCCGGGAAAA |
| | | (SEQ ID NO: 1135) |
| PSMB- | QSVLTQPPSVSAAPGQKVTISCSGSS | CAGTCTGTGCTGACCCAGCCTCCTTCTGTGTCTGCTGCTCCTGGCCAGAAAGTGACCATCTCCTGCTCCGGCT |
| 896 | SNIGINYVSWYQQLPGTAPKLLIYDN | CCTCCTCCAACATCGGCATCAACTACGTGTCCTGGTATCAGCAGCTGCCCGGCACAGCTCCCAAACTGCTGAT |
| | NKRPSGIPDRESGSKSGTSATLGITG | CTACGACAACAACAAGCGGCCCAGCGGCATCCCTGACAGATTCTCCGGATCTAAGTCCGGCACCTCTGCTACC |
| | LQTGDEADYYCGTWDSSLSAVVFGGG | CTGGGCATCACAGGATTGCAGACAGGCGACGAGGCCGACTACTATTGCGGCACCTGGGACTCTTCTCTGTCCG |
| | TKLTVLGGSEGKSSGSGSESKSTGGS | CCGTTGTTTTTGGCGGTGGCACCAAGCTGACAGTGCTCGGCGGCTCCGAGGGCAAGAGCAGCGGCAGCGGCAG |
| | EVQLVESGGGLVQPGGSLRLSCAASG | CGAGAGCAAGAGCACCGGCGGCAGCGAGGTGCAGCTGGTTGAATCTGGCGGAGGATTGGTTCAGCCTGGCGGT |
| | FTFSSYAMSWVRQAPGKGLEWVSAIS | TCTCTGAGACTGTCTTGTGCCGCTTCCGGCTTCACCTTCTCCAGCTACGCTATGTCCTGGGTCCGACAGGCTC |
| | GGIGSTYYADSVKGRFTISRDNSKNT | CTGGCAAAGGACTGGAATGGGTGTCCGCTATCTCTGGCGGCATCGGCTCTACCTACTACGCCGACTCTGTGAA |
| | LWLQMNSLRAEDTAVYYCAKDGVGAT | GGGCAGATTCACCATCAGCCGGGACAACTCCAAGAACACCCTGTGGCTGCAGATGAACTCCCTGAGAGCCGAG |
| | PYYFDYWGQGTLVTVSSEPKSSDKTH | GATACCGCCGTGTACTACTGTGCCAAAGATGGCGTGGGCGCTACCCCTTACTACTTCGATTATTGGGGCCAGG |
| | TCPPCPAPEAAGGPSVFLFPPKPKDT | GCACCCTGGTCACCGTTTCTTCTGAGCCCAAATCTAGCGACAAAACTCACACTTGTCCACCGTGCCCAGCACC |
| | LMISRTPEVTCVVVSVSHEDPEVKEN | TGAAGCAGCAGGGGGACCGTCAGTCTTCCTCTTCCCCCCAAAACCCAAGGACACCCTCATGATCTCCCGGACC |
| | WYVDGVEVHNAKTKPREEQYNSTYRV | CCTGAGGTCACATGCGTGGTGGTGAGCGTGAGCCACGAAGACCCTGAGGTCAAGTTCAACTGGTACGTGGACG |
| | VSVLTVLHQDWLNGKEYKCKVSNKAL | GCGTGGAGGTGCATAATGCCAAGACAAAGCCGCGGGAGGAGCAGTACAACAGCACGTACCGTGTGGTCAGCGT |
| | PAPIEKTISKAKGQPREPQVYVLPPS | CCTCACCGTCCTGCACCAGGACTGGCTGAATGGCAAGGAGTACAAGTGCAAGGTGTCCAACAAAGCCCTCCCA |
| | REEMTKNQVSLLCLVKGFYPSDIAVE | GCCCCCATCGAGAAAACCATCTCCAAAGCCAAAGGGCAGCCCCGAGAACCACAGGTGTACGTGCTGCCCCCAT |
| | WESNGQPENNYLTWPPVLDSDGSFFL | CCCGGGAGGAGATGACCAAGAACCAGGTCAGCCTGCTGTGCCTGGTCAAAGGCTTCTATCCCAGCGACATCGC |
| | YSKLTVDKSRWQQGNVFSCSVMHEAL | CGTGGAGTGGGAGAGCAATGGGCAGCCGGAGAACAACTACCTCACCTGGCCTCCCGTGCTGGACTCCGACGGC |
| | HNHYTQKSLSLSPGK | TCCTTCTTCCTCTACAGCAAGCTCACCGTGGACAAGTCCAGATGGCAGCAGGGGAACGTCTTCTCATGCTCCG |
| | (SEQ ID NO: 1136) | TGATGCATGAGGCTCTGCACAACCACTACACGCAGAAGTCTCTCTCCCTGTCTCCGGGAAAA |
| | | (SEQ ID NO: 1137) |
| PSMB- | QSVLTQPPSVSAAPGQKVTISCSGSS | CAGTCTGTGCTGACCCAGCCTCCTTCTGTGTCTGCTGCTCCTGGCCAGAAAGTGACCATCTCCTGCTCCGGCT |
| 897 | SNIGNNYVSWYQQLPGTAPKLLIYDN | CCTCCTCCAACATCGGCAACAACTACGTGTCCTGGTATCAGCAGCTGCCCGGCACAGCTCCCAAACTGCTGAT |
| | NKRPSGIPDRESGSKSGTSATLGITG | CTACGACAACAACAAGCGGCCCAGCGGCATCCCTGACAGATTCTCCGGATCTAAGTCCGGCACCTCTGCTACC |
| | LQTGDEADYYCGTWDSSLSAYVFGTG | CTGGGCATCACAGGATTGCAGACAGGCGACGAGGCCGACTACTATTGCGGCACCTGGGACTCTTCCCTGTCCG |
| | TKVTVLGGSEGKSSGSGSESKSTGGS | CTTATGTGTTTGGCACCGGCACCAAAGTGACCGTGTTGGGCGGCTCCGAGGGCAAGAGCAGCGGCAGCGGCAG |
| | EVQLVESGGGLVQPGGSLRLSCAASG | CGAGAGCAAGAGCACCGGCGGCAGCGAGGTGCAGCTGGTTGAATCTGGCGGAGGATTGGTTCAGCCTGGCGGT |
| | FTFSSYAMSWVRQAPGKGLEWVSAIS | TCTCTGAGACTGTCTTGTGCCGCTTCCGGCTTCACCTTCTCCAGCTACGCTATGTCCTGGGTCCGACAGGCTC |
| | GGSGSTYYADSVKGRFTISRDNSKNT | CTGGCAAAGGACTGGAATGGGTGTCCGCTATCTCTGGCGGATCCGGCTCTACCTACTACGCCGACTCTGTGAA |
| | LYLQMNSLRAEDSAVYYCAKDGVGAT | GGGCAGATTCACCATCAGCCGGGACAACTCCAAGAACACCCTGTACCTGCAGATGAACTCCCTGAGAGCCGAG |
| | PYYFDYWGQGTLVTVSSEPKSSDKTH | GACTCCGCCGTGTACTACTGTGCTAAAGATGGCGTGGGCGCTACCCCTTACTACTTCGATTATTGGGGCCAGG |
| | TCPPCPAPEAAGGPSVFLFPPKPKDT | GCACCCTGGTCACCGTTTCTTCTGAGCCCAAATCTAGCGACAAAACTCACACTTGTCCACCGTGCCCAGCACC |
| | LMISRTPEVTCVVVSVSHEDPEVKEN | TGAAGCAGCAGGGGGACCGTCAGTCTTCCTCTTCCCCCCAAAACCCAAGGACACCCTCATGATCTCCCGGACC |
| | WYVDGVEVHNAKTKPREEQYNSTYRV | CCTGAGGTCACATGCGTGGTGGTGAGCGTGAGCCACGAAGACCCTGAGGTCAAGTTCAACTGGTACGTGGACG |
| | VSVLTVLHQDWLNGKEYKCKVSNKAL | GCGTGGAGGTGCATAATGCCAAGACAAAGCCGCGGGAGGAGCAGTACAACAGCACGTACCGTGTGGTCAGCGT |
| | PAPIEKTISKAKGQPREPQVYVLPPS | CCTCACCGTCCTGCACCAGGACTGGCTGAATGGCAAGGAGTACAAGTGCAAGGTGTCCAACAAAGCCCTCCCA |
| | REEMTKNQVSLLCLVKGFYPSDIAVE | GCCCCCATCGAGAAAACCATCTCCAAAGCCAAAGGGCAGCCCCGAGAACCACAGGTGTACGTGCTGCCCCCAT |
| | WESNGQPENNYLTWPPVLDSDGSFFL | CCCGGGAGGAGATGACCAAGAACCAGGTCAGCCTGCTGTGCCTGGTCAAAGGCTTCTATCCCAGCGACATCGC |
| | YSKLTVDKSRWQQGNVFSCSVMHEAL | CGTGGAGTGGGAGAGCAATGGGCAGCCGGAGAACAACTACCTCACCTGGCCTCCCGTGCTGGACTCCGACGGC |
| | HNHYTQKSLSLSPGK | TCCTTCTTCCTCTACAGCAAGCTCACCGTGGACAAGTCCAGATGGCAGCAGGGGAACGTCTTCTCATGCTCCG |
| | (SEQ ID NO: 1138) | TGATGCATGAGGCTCTGCACAACCACTACACGCAGAAGTCTCTCTCCCTGTCTCCGGGAAAA |
| | | (SEQ ID NO: 1139) |
| PSMB- | QSVLTQPPSVSAAPGQKVTISCSGSS | CAGTCTGTGCTGACCCAGCCTCCTTCTGTGTCTGCTGCTCCTGGCCAGAAAGTGACCATCTCCTGCTCCGGCT |
| 898 | SNIGINYVSWYQQLPGTAPKLLIYDN | CCTCCTCCAACATCGGCATCAACTACGTGTCCTGGTATCAGCAGCTGCCCGGCACAGCTCCCAAACTGCTGAT |
| | NKRPSGIPDRESGSKSGTSATLGITG | CTACGACAACAACAAGCGGCCCAGCGGCATCCCTGACAGATTCTCCGGATCTAAGTCCGGCACCTCTGCTACC |
| | LQTGDEADYYCGTWDSSLSAVVFGGG | CTGGGCATCACAGGATTGCAGACAGGCGACGAGGCCGACTACTATTGCGGCACCTGGGACTCTTCTCTGTCCG |
| | TKLTVLGGSEGKSSGSGSESKSTGGS | CCGTTGTTTTTGGTGGAGGCACCAAGCTGACAGTGCTCGGCGGCTCCGAGGGCAAGAGCAGCGGCAGCGGCAG |
| | EVQLVESGGGLVQPGGSLRLSCAASG | CGAGAGCAAGAGCACCGGCGGCAGCGAGGTGCAGCTGGTTGAATCTGGCGGAGGATTGGTTCAGCCTGGCGGT |
| | FTFSSYAMSWVRQAPGKGLEWVSAIS | TCTCTGAGACTGTCTTGTGCCGCTTCCGGCTTCACCTTCTCCAGCTACGCTATGTCCTGGGTCCGACAGGCTC |
| | GGSGSTYYADSVKGRFTISRDNSKNT | CTGGCAAAGGACTGGAATGGGTGTCCGCTATCTCTGGCGGATCCGGCTCTACCTACTACGCCGACTCTGTGAA |
| | LYLQMNSLRAEDSAVYYCAKDGVGAT | GGGCAGATTCACCATCAGCCGGGACAACTCCAAGAACACCCTGTACCTGCAGATGAACTCCCTGAGAGCCGAG |
| | PYYFDYWGQGTLVTVSSEPKSSDKTH | GACTCCGCCGTGTACTACTGTGCTAAAGATGGCGTGGGCGCTACCCCTTACTACTTCGATTATTGGGGCCAGG |
| | TCPPCPAPEAAGGPSVFLFPPKPKDT | GCACCCTGGTCACCGTTTCTTCTGAGCCCAAATCTAGCGACAAAACTCACACTTGTCCACCGTGCCCAGCACC |
| | LMISRTPEVTCVVVSVSHEDPEVKEN | TGAAGCAGCAGGGGGACCGTCAGTCTTCCTCTTCCCCCCAAAACCCAAGGACACCCTCATGATCTCCCGGACC |
| | WYVDGVEVHNAKTKPREEQYNSTYRV | CCTGAGGTCACATGCGTGGTGGTGAGCGTGAGCCACGAAGACCCTGAGGTCAAGTTCAACTGGTACGTGGACG |
| | VSVLTVLHQDWLNGKEYKCKVSNKAL | GCGTGGAGGTGCATAATGCCAAGACAAAGCCGCGGGAGGAGCAGTACAACAGCACGTACCGTGTGGTCAGCGT |
| | PAPIEKTISKAKGQPREPQVYVLPPS | CCTCACCGTCCTGCACCAGGACTGGCTGAATGGCAAGGAGTACAAGTGCAAGGTGTCCAACAAAGCCCTCCCA |
| | REEMTKNQVSLLCLVKGFYPSDIAVE | GCCCCCATCGAGAAAACCATCTCCAAAGCCAAAGGGCAGCCCCGAGAACCACAGGTGTACGTGCTGCCCCCAT |
| | WESNGQPENNYLTWPPVLDSDGSFFL | CCCGGGAGGAGATGACCAAGAACCAGGTCAGCCTGCTGTGCCTGGTCAAAGGCTTCTATCCCAGCGACATCGC |
| | YSKLTVDKSRWQQGNVFSCSVMHEAL | CGTGGAGTGGGAGAGCAATGGGCAGCCGGAGAACAACTACCTCACCTGGCCTCCCGTGCTGGACTCCGACGGC |
| | HNHYTQKSLSLSPGK | TCCTTCTTCCTCTACAGCAAGCTCACCGTGGACAAGTCCAGATGGCAGCAGGGGAACGTCTTCTCATGCTCCG |
| | (SEQ ID NO: 1140) | TGATGCATGAGGCTCTGCACAACCACTACACGCAGAAGTCTCTCTCCCTGTCTCCGGGAAAA |
| | | (SEQ ID NO: 1141) |
| PSMA_ | EIVLTQSPGTLSVSPGERATLSCRAS | GAGATCGTGCTGACCCAGTCTCCTGGAACTCTGTCTGTGTCTCCTGGCGAGAGAGCTACCCTGTCTTGCAGAG |
| P75_ | QSVRSNLAWYQQKPGQAPRLLIYGAS | CCTCTCAGTCCGTGCGGTCTAACCTGGCCTGGTATCAGCAGAAACCTGGACAGGCCCCTAGACTGCTGATCTA |
| F01 | TRATGIPARFSGSGSGTEFTLTISSL | CGGCGCTTCTACCAGAGCCACAGGCATCCCTGCCAGATTTTCTGGCTCTGGCTCCGGCACCGAGTTCACCCTG |
| | QSEDFAVYYCHQYNDWPPYTFGQGTK | ACAATCAGTTCCCTGCAGTCCGAGGACTTCGCCGTGTACTACTGCCACCAGTACAACGACTGGCCTCCTTACA |
| | LEIKGGSEGKSSGSGSESKSTGGSQV | CCTTTGGCCAGGGCACCAAGCTGGAAATCAAAGGCGGCTCCGAGGGCAAGAGCAGCGGCAGCGGCAGCGAGAG |
| | QLQESGGGVVQPGRSLRLSCAASGFT | CAAGAGCACCGGCGGCAGCCAAGTTCAGTTGCAAGAATCTGGTGGCGGCGTGGTGCAGCCTGGAAGATCTCTG |
| | FSTYGMHWVRQAPGKGLEWVAFISYD | AGACTGTCTTGTGCCGCCTCCGGCTTCACCTTCTCCACCTATGGAATGCATTGGGTCCGACAGGCTCCCGGCA |
| | GSNKYYADSVKGRFTISRDNSKHTLY | AAGGACTGGAATGGGTCGCCTTCATCTCCTACGACGGCTCCAACAAGTACTACGCCGACTCCGTGAAGGGCAG |
| | LQMNSLRAEDTAVYYCAGRDNLRFLE | ATTCACCATCTCTCGGGACAACTCCAAGCACACCCTGTACCTGCAGATGAACTCCCTGAGAGCCGAGGATACC |
| | WFMDVWGQGTTVTVSSEPKSSDKTHT | GCTGTGTACTATTGCGCCGGCAGAGACAACCTGCGGTTCCTGGAATGGTTCATGGATGTGTGGGGCCAGGGAA |
| | CPPCPAPEAAGGPSVFLFPPKPKDTL | CCACCGTGACAGTTAGTTCTGAGCCCAAATCTAGCGACAAAACTCACACATGTCCACCGTGCCCAGCACCTGA |
| | MISRTPEVTCVVVSVSHEDPEVKENW | AGCAGCAGGGGGACCGTCAGTCTTCCTCTTCCCCCCAAAACCCAAGGACACCCTCATGATCTCCCGGACCCCT |
| | YVDGVEVHNAKTKPREEQYNSTYRVV | GAGGTCACATGCGTGGTGGTGAGCGTGAGCCACGAAGACCCTGAGGTCAAGTTCAACTGGTACGTGGACGGCG |
| | SVLTVLHQDWLNGKEYKCKVSNKALP | TGGAGGTGCATAATGCCAAGACAAAGCCGCGGGAGGAGCAGTACAACAGCACGTACCGTGTGGTCAGCGTCCT |
| | APIEKTISKAKGQPREPQVYVLPPSR | CACCGTCCTGCACCAGGACTGGCTGAATGGCAAGGAGTACAAGTGCAAGGTCTCCAACAAAGCCCTCCCAGCC |
| | EEMTKNQVSLLCLVKGFYPSDIAVEW | CCCATCGAGAAAACCATCTCCAAAGCCAAAGGGCAGCCCCGAGAACCACAGGTGTACGTGCTGCCCCCATCCC |
| | ESNGQPENNYLTWPPVLDSDGSFFLY | GGGAGGAGATGACCAAGAACCAGGTCAGCCTGCTGTGCCTGGTCAAAGGCTTCTATCCCAGCGACATCGCCGT |
| | SKLTVDKSRWQQGNVFSCSVMHEALH | GGAGTGGGAGAGCAATGGGCAGCCGGAGAACAACTACCTCACCTGGCCTCCCGTGCTGGACTCCGACGGCTCC |
| | NHYTQKSLSLSPG | TTCTTCCTCTACAGCAAGCTCACCGTGGACAAGTCTAGATGGCAGCAGGGGAACGTCTTCTCATGCTCCGTGA |
| | (SEQ ID NO: 1252) | TGCATGAGGCTCTGCACAACCACTACACGCAGAAGAGCCTCTCCCTGTCTCCGGGT |
| | | (SEQ ID NO: 1502) |
| PSMA_ | QSVLTQPPSASGTPGQRVTISCSGSS | CAGTCCGTGCTGACCCAGCCTCCTTCTGCTTCTGGAACACCTGGCCAGAGAGTGACCATCTCCTGCTCCGGCT |
| P70_ | SNIGSNTVNWYQQLPGTAPKLLIYSS | CCTCCTCCAACATCGGCTCTAACACCGTGAACTGGTATCAGCAGCTGCCCGGCACAGCCCCTAAACTGCTGAT |
| F02 | NQRPSGVPDRESGSKSGTSASLAISG | CTACTCTTCCAACCAGCGGCCTTCTGGCGTGCCCGATAGATTCTCTGGCTCCAAGTCTGGCACCTCCGCTAGC |
| | LQSEDEADYYCAAWDDSLNGVVFGGG | CTGGCTATTTCTGGCCTGCAGTCTGAGGACGAGGCCGATTACTACTGTGCCGCCTGGGATGATTCTCTGAACG |
| | TKLTVLGGSEGKSSGSGSESKSTGGS | GCGTTGTGTTTGGCGGAGGCACCAAATTGACAGTTCTTGGCGGCTCCGAGGGCAAGAGCAGCGGCAGCGGCAG |
| | EVQLLESGPGLVKPSETLSLTCTVSG | CGAGAGCAAGAGCACCGGCGGCAGCGAAGTTCAGCTGTTGGAATCTGGACCTGGCCTGGTCAAGCCTTCCGAG |
| | GSIISYYWSWIRQPAGKGLEWIGRIY | ACACTGTCTCTGACCTGTACCGTGTCCGGCGGCTCCATCATCTCCTACTACTGGTCCTGGATCAGACAGCCTG |
| | SSGSTNYNPSLKSRVTMSVDTSKNQF | CCGGCAAAGGACTGGAATGGATCGGCAGAATCTACTCCTCCGGCAGCACCAACTACAACCCCAGCCTGAAGTC |
| | SLKLSSVTAADTAVYYCAKVGVWPGA | CCGCGTGACCATGTCTGTGGACACCTCCAAGAACCAGTTCTCCCTGAAGCTGTCCTCTGTGACCGCCGCTGAT |
| | FDIWGQGTMVTVSSEPKSSDKTHTCP | ACCGCTGTGTACTACTGCGCTAAAGTCGGAGTGTGGCCTGGCGCCTTTGATATCTGGGGACAGGGCACAATGG |
| | PCPAPEAAGGPSVFLFPPKPKDTLMI | TCACCGTGTCCTCT |
| | SRTPEVTCVVVSVSHEDPEVKENWYV | (SEQ ID NO: 1503) |
| | DGVEVHNAKTKPREEQYNSTYRVVSV | |
| | LTVLHQDWLNGKEYKCKVSNKALPAP | |
| | IEKTISKAKGQPREPQVYVLPPSREE | |
| | MTKNQVSLLCLVKGFYPSDIAVEWES | |
| | NGQPENNYLTWPPVLDSDGSFFLYSK | |
| | LTVDKSRWQQGNVFSCSVMHEALHNH | |
| | YTQKSLSLSPG | |
| | (SEQ ID NO: 1264) | |

### Example 3: Characterization of Anti-PSMA Antibodies

### Example 3.1: Analytical Characterization of Purified Anti-PSMA Antibodies

The protein concentration for each purified mAb was determined by measuring the absorbance at 280 nm on a NANODROP1000 spectrophotometer or TRINEAN DROPSENSE96 multichannel spectrophotometer and calculated using the extinction coefficient based on the amino acid sequence. SE HPLC of the purified antibodies was performed by running samples on a TOSOH TSKgel BioAssist G3SWxl column, in 0.2 M Na Phosphate pH 6.8 at 1 mL/min on a Waters Alliance HPLC for 20 min. The column effluent was monitored by absorbance at 280 nm. Bispecific antibodies (bsAbs) were analyzed by Intact Mass Analysis to determine appropriate formation of heterodimers.

### Example 3.2: Anti-PSMA Antibodies Bind PSMA with High Affinity

The binding affinity of selected anti-PSMA antibodies to the recombinant human, cynomolgus or mouse PSMA ECD (PSMW39, PSMW1 and PSMW29, respectively) was determined by surface plasmon resonance (SPR) using a BIACORE 8K instrument (ELN PSMA-00702). The antibodies were captured on a goat anti-Fc antibody-modified C1 chip and titrated with 3-fold serial dilutions of PSMA antigen spanning concentrations of 1 nM to 11.1 nM. The association and dissociation were monitored for 3 and 15 minutes, respectively, using a flow rate of 50 μL/min. Raw binding data was referenced by subtracting the analyte binding signals from blanks and analyzed using a 1:1 Langmuir binding model using the Biacore Insight evaluation software to obtain the kinetics, which were used to calculate the binding affinity. The kinetic parameter of binding of selected antibodies are shown in Table 13. The anti-PSMA were captured using an anti-human Fc antibody and the antigens were injected in solution. The anti-PSMA antibodies were found to bind both human PSMA and cyno PSMA with picomolar to nanomolar affinities.

TABLE 13
Affinities (KD) for the interaction of anti-PSMA antibodies with Human,
cyno or mouse PSMA as obtained by the BIACORE (SPR) method.

| Antibody | Human, K_{D} (M) | Cyno, K_{D} (M) | Mouse, K_{D} (M) |
|---|---|---|---|
| PSMB889 | ≤2.27E−11 | Poor fit | Low/no binding |
| PSMB890 | poor fit | Low/No binding | Low/No binding |
| PSMB891 | ≤2.53E−10 | ≤2.10E−10 | Low/No binding |
| PSMB892 | 3.22E−09 | 5.67E−09 | 4.04E−10 |
| PSMB893 | Low/No binding | 2.60E−07 | Low/No binding |
| PSMB894 | No capture | No capture | No capture |
| PSMB895 | 7.04E−10 | 2.83E−09 | Low/No binding |
| PSMB896 | ≤8.76E−11 | 6.58E−10 | 1.36E−09 |
| PSMB897 | 1.28E−09 | 3.93E−09 | Low/No binding |
| PSMB898 | 3.33E−10 | 1.19E−09 | Low/No binding |
| PSMB899 | 1.32E−10 | 1.47E−09 | Low/No binding |

### Example 3.3: Thermal Stability of Anti-PSMA Antibodies

The thermal stability (conformational stability) of the anti-PSMA antibodies was determined by nanoDSF method using a Prometheus instrument. Measurements were made by loading sample into 24 well capillary from a 384 well sample plate. Duplicate runs were performed. The thermal scans span from 20° C. to 95° C. at a rate of 1.0° C./minute. The data was processed to obtain integrated data and first derivation analysis for 330 nm, 350 nm, Ratio 330/350, and scatter data from which thermal transitions, onset of unfolding, Tm and Tagg were obtained. Thermal unfolding parameters, Tm or midpoint temperature of thermal unfolding and Tagg or aggregation temperature of selected anti-PSMA antibodies are shown in Table 14. PSMB889, PSMB890, PSMB891, PSMB892, PSMB895, PSMB896, PSMB897 and PSMB898 showed similar temperatures of unfolding between 68 and 69.9° C. PSMB895, PSMB896, PSMB897 and PSMB898 showed the highest temperature of unfolding ranging from 80.3° C. to 94.7° C.

TABLE 14
Thermal stability data for anti-PSMA antibodies
as obtained using a nanoDSF instrument.

| Antibody | T_{agg} | Tₘ1 | Tₘ2 |
|---|---|---|---|
| PSMB889 | 68.6° C. | 69.1° C. | |
| PSMB890 | 68.6° C | 68.5° C. | |
| PSMB891 | 75.2° C. | 69.2° C. | 76.0° C. |
| PSMB892 | 69.0° C. | 68.0° C. | |
| PSMB893 | 68.3º C. | 60.7° C. | 67.8° C. |
| PSMB894 | 68.5° C. | 53.8° C. | 66.2° C. |
| PSMB895 | 84.7º C. | 69.3º C. | 84.4º C. |
| PSMB896 | 80.3º C. | 69.9º C. | 81.0° C. |
| PSMB897 | 85.2° C. | 68.6° C. | 86.1º C. |
| PSMB898 | 83.2° C . | 69.1° C. | 83.1° C. |
| PSMB899 | 78.0° C. | 69.0° C. | 79.1º C. |

### Example 3.4: Binding of Anti-PSMA Antibodies on PSMA+ Cells

Selected anti-PSMA antibodies were evaluated for binding to commercial prostate cancer cell lines. C4-2B cells were originally developed at MD Anderson and are derived from LNCaP FGC grown in vivo and metastasize to bone marrow (Thalmann, et al 1994, Cancer Research 54, 2577-81). 22Rv1 cells are a human prostate carcinoma epithelial cell line derived from a xenograft that was serially propagated in mice after castration-induced regression and relapse of the parental, androgen-dependent CWR22 xenograft. 22RV1 and C4-2B cells were plated at 50,000 cells per well in 50 μl of assay medium (RPMI, 10% HI FBS) in V bottom plates. Serial dilutions of antibodies were prepared in assay medium with 50 μl of antibody dilutions added to the plates containing cells. The plates were incubated for 60 min at 37° C. at which time 100 μl of staining buffer (Becton Dickinson Cat #554657) was added to all wells of each plate. The plates were centrifuged at 300×G for 5 minutes and the medium was removed from the wells. 200 μl of staining buffer was added to all wells of each plate. The plates were centrifuged at 300×G for 5 minutes and the medium was removed from the wells. 50 μl of 2 μg/ml AlexaFluor647-labeled goat anti-human Fc was added to all wells of the plates and the plates were incubated for 30 minutes at 4° C. 150 μl of staining buffer was added to all wells of each plate. The plates were centrifuged at 300×G for 5 minutes and the medium was removed from the wells. 200 μl of running buffer (Staining buffer plus 1 mM EDTA, 0.1% pluronic acid was added to all wells of the plates. The plates were centrifuged at 300×G for 5 minutes and the medium was removed from the wells. 30 μl running buffer was added to all wells with cells and the plates were analyzed on the IQUE PLUS instrument (Sartorius). Briefly, cells were gated on a FCS vs. SSC gate to eliminate cellular debris, then the cell populations were gated on singlet cells. Antibody binding was assessed in the red laser channel. Signal (Mab plus secondary antibody) to background (secondary antibody only) ratios were calculated for each plate and the resultant data was plotted vs. MAB concentration in GeneData Screener using 4 parameter curve fitting to generate EC50 values. Table 15 shows EC50 values of anti-PSMA antibodies binding to PSMA+ cell lines.

TABLE 15
EC50 values of anti-PSMA antibodies binding to PSMA-expressing
cell lines 22RV1 and C4-2B measured by flow cytometry assays.

| Antibody | 22RV1 EC50 [M] | C4-2B EC50 [M] |
|---|---|---|
| PSMB889 | 9.80E−11 | 4.42E−10 |
| PSMB890 | 1.71E−09 | 9.41E−10 |
| PSMB891 | 5.54E−10 | 1.99E−09 |
| PSMB892 | 6.00E−08 | 2.24E−08 |
| PSMB893 | 6.00E−08 | 6.00E−08 |
| PSMB894 | 6.75E−09 | 5.35E−08 |
| PSMB895 | 1.71E−09 | 5.09E−09 |
| PSMB896 | 1.24E−09 | 2.58E−08 |
| PSMB897 | 2.26E−09 | 4.76E−09 |
| PSMB898 | 2.07E−09 | 6.53E−09 |
| PSMB899 | 1.02E−09 | 5.58E−09 |

### Example 4: Generation of Anti-CD3 Antibodies

### Example 4.1: Generation of CD3B376 and CD3B450

Ablexis AlivaMab or OMT transgenic mice can be used for the production of antibodies. The generation of anti-CD3 antibody CD3B376 and CD3B450 has been described in US Publ. No. 20200048349, which is incorporated by reference in its entirety. CD3B376 and CD3B450 have the same CDR sequences as shown in Tables 16-21 below.

### Example 4.2: Generation of CD3W245

Anti-CD3 CD3W245 was generated using the Ablexis Transgenic mice technology. Ablexis mice were immunized with TRCWS (SEQ ID NO:1142). TRCWS is comprised of the extracellular region of CD36 fused by a 26 amino acid linker to the extracellular region of CD3E. This polypeptide has at its C-terminus a human IgG1 Fc domain with a C-terminal Avi-tag used for site-specific biotinylation).

TRCW5
SEQ ID NO: 1142
FKIPIEELEDRVFVNCNTSITWVEGTVGTLLSDITRLDLGKRILDPRGIY
RCNGTDIYKDKESTVQVHYRMGSADDAKKDAAKKDDAKKDDAKKDGSDGN
EEMGGITQTPYKVSISGTTVILTCPQYPGSEILWQHNDKNIGGDEDDKNI
GSDEDHLSLKEFSELEQSGYYVCYPRGSKPEDANFYLYLRARVSPPSPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGKGGGLNDIFEAQKIEWHE,

Mice were immunized twice weekly for the duration of 7 weeks. On day 42, mice were boosted for hybridoma fusion by administration of 50 μg TRCWS and 50 μg CD40 mAb spread over 8 sites, including 6 subcutaneous and 2 intradermal injections. For a final boost, mice received 20 μL injections of Jurkat cells, a T cell line which endogenously expresses the T cell receptor complex, including CD3E (Schneider et al (1977) Int. J. Cancer, 19 (5): 621-6), at 4.74×10⁷ cells/mL.

Mice hhybridoma were assayed by ELISA for binding to TRCWS using TRCWS either non-specifically immobilized on the plate (ELISA, Thermo cat. #34022) or by streptavidin conjugation to biotinylated-TRCWS (SPARCL ELISA, Lumigen). The two assay formats resulted in 426 hits (264 hits from ELISA, 194 from SPARCL ELISA, 70 hits were identified in both assays). Of these 426 initial hits, 49 ELISA and 32 SPARCL ELISA hits were confirmed. The hybridoma fusions corresponding to the positive binders were refed and tested for their abilities to bind Jurkat cells by flow cytometry. Three antibodies, clone 003_F12, clone 036_E10 and clone 065 D03, showed significant binding to Jurkat cells, endogenously expressing CD3. Next, these three clones were screened for their abilities to bind primary human and cyno T cells.

Clone 065_D03 was selected for further development. The variable region of the Clone 065_D03 was cloned into an IgG1 backbone, resulting in the antibody termed CD3B815.

CD3B815 Heavy Chain
SEQ ID NO: 1143
EVOLVESGGGLVKPGGSLRLSCAASGFTFSRYNMNWVRQAPGKGLEWVSS
ISTSSNYIYYADSVKGRFTFSRDNAKNSLDLQMSGLRAEDTAIYYCTRGW
GPFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK,
CD3B815 Light Chain
SEQ ID NO: 1144
DILLTQSPGILSVSPGERVSFSCRARQSIGTAIHWYQQRINGSPRLLIKY
ASESISGIPSRFSGSGSGTDFTLTINSVESEDIADYYCQQSNSWPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC,

The light chain (LC) of the v-region of CD3B815 was humanized in scFv format. Briefly, the LC from CD3B815 was grafted onto the human IGKV1-39*01-IGKJ2*01 germline and two positions (Y49K and L78V) were identified for human to mouse back mutations. This resulted in variants, having either Y49K, L78V, or both Y49K and L78V. The LC also presented a risk of deamination because of a NS motif at positions 92-93. These modifications resulted into an antibody termed CD3W245 scFv generated by grafting the CDRs of CD3B815 onto IGKV1D-39*01 and introducing the mutations Y49K and N92G into the VL.

### Example 4.3: Generation of CD3B2030 and CD3B2030-N106A

The publicly available mouse Cris7 antibody, specific to human CD3ε (Alberola-Ila, J. et al., J Immunol 146, 1085-1092 (1991)) was used to generate additional exemplary CD3 binding domain provided herein. Murine Cris-7 was humanized in the single-chain fragment variable-domain (scFv) format. Two human heavy variable-domain (Hv) germline sequences and two human light variable-domain (Lv) germline sequences were selected for the antibody humanization: IGHV1-69*02-IGHJ1-01 and IGHV5-10-1*01-IIGHJ1-01 for Hv and IGKV3-11*02-IGKJ4-01 or the IGKV1-39*01-IGKJ4-01 germline for LvThe CDR-grafted sequences were generated by grafting murine Cris7 CDRs onto these sequences while limiting the number of back mutations to enhance stability. The CDR-grafted v-regions were then expressed in E. coli in scFv format in both the heavy chain-linker-light chain (HL) and in the light chain-linker-heavy chain (LH) orientations. A matrix of Hv and Lv pairings was evaluated in scFv-format in both orientations of Lv followed by Hv or Hv followed by Lv and with a flexible linker between the variable domains. The IGHV1-69*02-IGHJ1-01 heavy chain germline with IGKV3-11*02-IGKJ4-01 light chain germline grafted construct in the heavy-light orientation exhibited the best expression, binding profile, and potential differentiation, and was chosen for human framework optimization.

Several sites in the VH, which may influence stability of the molecule were identified and thus selected for library-based mouse back-mutagenesis. In one VH library, 4 sites (M48I, A60N, V67A, and I69L—Kabat numbering) were mutated in binary libraries and R94 (Kabat numbering) was mutated to S, V, L, K, T, R, I, or Y generating a total of 128 variants. In a second library, 9 sites (K12A, V20M, R38K, M48I, A60N, R66, V67A, I69L, and R94S—Kabat numbering) were mutated in a binary library generating a total of 512 variants. Analogously, two libraries were generated for the VL sequence, by identifying sites which may influence stability of the molecule. In library 1, no changes were made to the LC. In library 2, 11 sites were selected for mouse back-mutagenesis in binary fashion (L11M, L13A, A19V, L21M, Q42T, A43S, L46R, L47W, I58V, F71Y, and L78M) for a total of 2048 variants.

Clones exhibiting binding to CD36E greater than the murine parent were selected for sequencing and exposed to additional assay including titration, thermal stress and cell binding. Cell binding was performed against Pan T-cells and Jurkat-CD3-negative cells to look for increase in non-specific binding when thermally stressed. Molecules exhibiting an increase in binding to the negative cell line when thermally stressed were not chosen for additional characterization. Four humanized matrixed clones retained binding after 65° C. heat shock and were selected. These clones include CD3B2030, whose CDR, VH and VL sequences are included in the tables below.

CD3B2030 contains an NG motif within the Heavy Variable CDR3 (HCDR3). To mitigate this Post Translational Modification (PTM) risk, which could negatively impact potency of the T cell re-directing function, the N106A mutation was introduced using standard molecular biology techniques, generating the CD3B2030-N106A variant.

Sequences of representative CD3 antibodies are provided in Tables 16-21. Table 16 provides VH amino acid sequences, VL sequences, heavy chain sequences, and light chain sequences of various CD3 antibodies. VH CDR and VL CDR sequences are provided in Table 17 (Kabat), Table 18 (Chothia), Table 19 (AbM), Table 20 (Contact), and Table 21 (IMGT). Numbers beneath each sequence in the Tables denote corresponding SEQ ID NOs.

TABLE 16
CD3 Antibody VH, VL, HC and LC Amino Acid Sequences

| | Protein | | | | |
|---|---|---|---|---|---|
| | Name | VH AA | | | Light Chain AA |
| # | (Target) | sequence | VL AA sequence | Heavy Chain AA sequence | sequence |
| 25 | CD3B376+ | QVQLQQSGPRLVR | QSALTQPASVSGSPGQ | QVQLQQSGPRLVRPSQTLSLTCAISGDSVENNNAAWSWIRQ | QSALTQPASVSGSPGQ |
| | K477 | PSQTLSLTCAISG | SITISCTGTSSNIGTY | SPSRGLEWLGRTYYRSKWLYDYAVSVKSRITVNPDTSRNQF | SITISCTGTSSNIGTY |
| | (CD3) | DSVENNNAAWSWI | KFVSWYQQHPDKAPKV | TLQLNSVTPEDTALYYCARGYSSSFDYWGQGTLVTVSSAST | KFVSWYQQHPDKAPKV |
| | AKA | RQSPSRGLEWLGR | LLYEVSKRPSGVSSRF | KGPSVFPLAPSSKSTSGGTAALGCLVKDYFPEPVTVSWNSG | LLYEVSKRPSGVSSRF |
| | CD3B2197 | TYYRSKWLYDYAV | SGSKSGNTASLTISGL | ALTSGVHTFPAVLQSSGLYSLSSVVTVPSSSLGTQTYICNV | SGSKSGNTASLTISGL |
| | | SVKSRITVNPDTS | QAEDQADYHCVSYAGS | NHKPSNTKVDKKVEPKSCDKTHTCPPCPAPEAAGGPSVELF | QAEDQADYHCVSYAGS |
| | | RNQFTLQLNSVTP | GTLLFGGGTKLTVL | PPKPKDTLMISRTPEVTCVVVSVSHEDPEVKFNWYVDGVEV | GTLLFGGGTKLTVLGQ |
| | | EDTALYYCARGYS | | HNAKTKPREEQYNSTYRVVSVLTVLHQDWLNGKEYKCKVSN | PKAAPSVTLFPPSSEE |
| | | SSFDYWGQGTLVT | | KALPAPIEKTISKAKGQPREPQVYTLPPSREEMTKNQVSLT | LQANKATLVCLISDFY |
| | | VSS | | CLVKGFYPSDIAVEWESNGQPENNYKTTPPVLDSDGSFLLY | PGAVTVAWKADSSPVK |
| | | | | SKLTVDKSRWQQGNVFSCSVMHEALHNHYTQKSLSLSPGK | AGVETTTPSKQSNNKY |
| | | | | | AASSYLSLTPEQWKSH |
| | | | | | RSYSCQVTHEGSTVEK |
| | | | | | TVAPTECS |
| | | 847 | 848 | 849 | 850 |
| 26 | CD3B376- | QVQLQQSGPRLVR | QSALTQPASVSGSPGQ | QVQLQQSGPRLVRPSQTLSLTCAISGDSVENNNAAWSWIRQ | (see above) |
| | K477 | PSQTLSLTCAISG | SITISCTGTSSNIGTY | SPSRGLEWLGRTYYRSKWLYDYAVSVKSRITVNPDTSRNQF | |
| | (CD3) | DSVENNNAAWSWI | KFVSWYQQHPDKAPKV | TLQLNSVTPEDTALYYCARGYSSSFDYWGQGTLVTVSSAST | |
| | AKA | RQSPSRGLEWLGR | LLYEVSKRPSGVSSRF | KGPSVFPLAPSSKSTSGGTAALGCLVKDYFPEPVTVSWNSG | |
| | CD3B891 | TYYRSKWLYDYAV | SGSKSGNTASLTISGL | ALTSGVHTFPAVLQSSGLYSLSSVVTVPSSSLGTQTYICNV | |
| | | SVKSRITVNPDTS | QAEDQADYHCVSYAGS | NHKPSNTKVDKKVEPKSCDKTHTCPPCPAPEAAGGPSVFLE | |
| | | RNQFTLQLNSVTP | GTLLFGGGTKLTVL | PPKPKDTLMISRTPEVTCVVVSVSHEDPEVKENWYVDGVEV | |
| | | EDTALYYCARGYS | | HNAKTKPREEQYNSTYRVVSVLTVLHQDWLNGKEYKCKVSN | |
| | | SSFDYWGQGTLVT | | KALPAPIEKTISKAKGQPREPQVYTLPPSREEMTKNQVSLT | |
| | | VSS | | CLVKGFYPSDIAVEWESNGQPENNYKTTPPVLDSDGSFLLY | |
| | | | | SKLTVDKSRWQQGNVFSCSVMHEALHNHYTQKSLSLSPG | |
| | | 847 | 848 | 883 | 850 |
| 27 | CD3B450+ | QVQLQQSGPGLVK | QSALTQPASVSGSPGQ | QVQLQQSGPGLVKPSQTLSLTCAISGDSVENNNAAWSWIRQ | QSALTQPASVSGSPGQ |
| | K477 | PSQTLSLTCAISG | SITISCTGTSSNIGTY | SPSRGLEWLGRTYYRSKWLYDYAVSVKSRITINPDTSKNQF | SITISCTGTSSNIGTY |
| | (CD3) | DSVENNNAAWSWI | KFVSWYQQHPGKAPKV | SLQLNSVTPEDTAVYYCARGYSSSFDYWGQGTLVTVSSAST | KFVSWYQQHPGKAPKV |
| | AKA | RQSPSRGLEWLGR | MIYEVSKRPSGVSNRF | KGPSVFPLAPSSKSTSGGTAALGCLVKDYFPEPVTVSWNSG | MIYEVSKRPSGVSNRF |
| | CD3B2200 | TYYRSKWLYDYAV | SGSKSGNTASLTISGL | ALTSGVHTFPAVLQSSGLYSLSSVVTVPSSSLGTQTYICNV | SGSKSGNTASLTISGL |
| | | SVKSRITINPDTS | QAEDEADYYCVSYAGS | NHKPSNTKVDKKVEPKSCDKTHTCPPCPAPEAAGGPSVELF | QAEDEADYYCVSYAGS |
| | | KNQFSLQLNSVTP | GTLLFGGGTKLTVL | PPKPKDTLMISRTPEVTCVVVSVSHEDPEVKENWYVDGVEV | GTLLFGGGTKLTVLGQ |
| | | EDTAVYYCARGYS | | HNAKTKPREEQYNSTYRVVSVLTVLHQDWLNGKEYKCKVSN | PKAAPSVTLEPPSSEE |
| | | SSFDYWGQGTLVT | | KALPAPIEKTISKAKGQPREPQVYTLPPSREEMTKNQVSLT | LQANKATLVCLISDFY |
| | | VSS | | CLVKGFYPSDIAVEWESNGQPENNYKTTPPVLDSDGSFLLY | PGAVTVAWKADSSPVK |
| | | | | SKLTVDKSRWQQGNVFSCSVMHEALHNHYTQKSLSLSPGK | AGVETTTPSKQSNNKY |
| | | | | | AASSYLSLTPEQWKSH |
| | | | | | RSYSCQVTHEGSTVEK |
| | | | | | TVAPTECS |
| | | 915 | 916 | 917 | 918 |
| 28 | CD3B450- | QVQLQQSGPGLVK | QSALTQPASVSGSPGQ | QVQLQQSGPGLVKPSQTLSLTCAISGDSVENNNAAWSWIRQ | (see above) |
| | K477 | PSQTLSLTCAISG | SITISCTGTSSNIGTY | SPSRGLEWLGRTYYRSKWLYDYAVSVKSRITINPDTSKNQF | |
| | (CD3) | DSVENNNAAWSWI | KFVSWYQQHPGKAPKV | SLQLNSVTPEDTAVYYCARGYSSSFDYWGQGTLVTVSSAST | |
| | AKA | RQSPSRGLEWLGR | MIYEVSKRPSGVSNRF | KGPSVFPLAPSSKSTSGGTAALGCLVKDYFPEPVTVSWNSG | |
| | CD3B2186 | TYYRSKWLYDYAV | SGSKSGNTASLTISGL | ALTSGVHTFPAVLQSSGLYSLSSVVTVPSSSLGTQTYICNV | |
| | | SVKSRITINPDTS | QAEDEADYYCVSYAGS | NHKPSNTKVDKKVEPKSCDKTHTCPPCPAPEAAGGPSVELF | |
| | | KNQFSLQLNSVTP | GTLLFGGGTKLTVL | PPKPKDTLMISRTPEVTCVVVSVSHEDPEVKENWYVDGVEV | |
| | | EDTAVYYCARGYS | | HNAKTKPREEQYNSTYRVVSVLTVLHQDWLNGKEYKCKVSN | |
| | | SSFDYWGQGTLVT | | KALPAPIEKTISKAKGQPREPQVYTLPPSREEMTKNQVSLT | |
| | | VSS | | CLVKGFYPSDIAVEWESNGQPENNYKTTPPVLDSDGSFLLY | |
| | | | | SKLTVDKSRWQQGNVFSCSVMHEALHNHYTQKSLSLSPG | |
| | | 915 | 916 | 951 | 918 |
| 29 | CD3W245 | EVQLVESGGGLVK | DIQMTQSPSSLSASVG | EVQLVESGGGLVKPGGSLRLSCAASGFTFSRYNMNWVRQAP | DIQMTQSPSSLSASVG |
| | (-K477) | PGGSLRLSCAASG | DRVTITCRARQSIGTA | GKGLEWVSSISTSSNYIYYADSVKGRFTFSRDNAKNSLDLQ | DRVTITCRARQSIGTA |
| | (CD3) | FTFSRYNMNWVRQ | IHWYQQKPGKAPKLLI | MSGLRAEDTAIYYCTRGWGPFDYWGQGTLVTVSSASTKGPS | IHWYQQKPGKAPKLLI |
| | AKA | APGKGLEWVSSIS | KYASESISGVPSRESG | VFPLAPSSKSTSGGTAALGCLVKDYFPEPVTVSWNSGALTS | KYASESISGVPSRESG |
| | CD3W245 | TSSNYIYYADSVK | SGSGTDFTLTISSLQP | GVHTFPAVLQSSGLYSLSSVVTVPSSSLGTQTYICNVNHKP | SGSGTDFTLTISSLQP |
| | or | GRFTFSRDNAKNS | EDFATYYCQQSGSWPY | SNTKVDKKVEPKSCDKTHTCPPCPAPEAAGGPSVFLFPPKP | EDFATYYCQQSGSWPY |
| | CD3B2183 | LDLQMSGLRAEDT | TFGQGTKLEIK | KDTLMISRTPEVTCVVVSVSHEDPEVKFNWYVDGVEVHNAK | TFGQGTKLEIKRTVAA |
| | | AIYYCTRGWGPFD | | TKPREEQYNSTYRVVSVLTVLHQDWLNGKEYKCKVSNKALP | PSVFIFPPSDEQLKSG |
| | | YWGQGTLVTVSS | | APIEKTISKAKGQPREPQVYTLPPSREEMTKNQVSLTCLVK | TASVVCLLNNFYPREA |
| | | | | GFYPSDIAVEWESNGQPENNYKTTPPVLDSDGSFLLYSKLT | KVQWKVDNALQSGNSQ |
| | | | | VDKSRWQQGNVFSCSVMHEALHNHYTQKSLSLSPG | ESVTEQDSKDSTYSLS |
| | | | | | STLTLSKADYEKHKVY |
| | | | | | ACEVTHQGLSSPVTKS |
| | | | | | ENRGEC |
| | | 983 | 984 | 985 | 986 |
| 30 | CD3W245+ | EVQLVESGGGLVK | DIQMTQSPSSLSASVG | EVQLVESGGGLVKPGGSLRLSCAASGFTFSRYNMNWVRQAP | (see above) |
| | K477 | PGGSLRLSCAASG | DRVTITCRARQSIGTA | GKGLEWVSSISTSSNYIYYADSVKGRFTESRDNAKNSLDLQ | |
| | (CD3) | FTFSRYNMNWVRQ | IHWYQQKPGKAPKLLI | MSGLRAEDTAIYYCTRGWGPFDYWGQGTLVTVSSASTKGPS | |
| | AKA | APGKGLEWVSSIS | KYASESISGVPSRESG | VFPLAPSSKSTSGGTAALGCLVKDYFPEPVTVSWNSGALTS | |
| | N/A | TSSNYIYYADSVK | SGSGTDFTLTISSLQP | GVHTFPAVLQSSGLYSLSSVVTVPSSSLGTQTYICNVNHKP | |
| | | GRFTFSRDNAKNS | EDFATYYCQQSGSWPY | SNTKVDKKVEPKSCDKTHTCPPCPAPEAAGGPSVFLFPPKP | |
| | | LDLQMSGLRAEDT | TFGQGTKLEIK | KDTLMISRTPEVTCVVVSVSHEDPEVKFNWYVDGVEVHNAK | |
| | | AIYYCTRGWGPFD | | TKPREEQYNSTYRVVSVLTVLHQDWLNGKEYKCKVSNKALP | |
| | | YWGQGTLVTVSS | | APIEKTISKAKGQPREPQVYTLPPSREEMTKNQVSLTCLVK | |
| | | | | GFYPSDIAVEWESNGQPENNYKTTPPVLDSDGSFLLYSKLT | |
| | | | | VDKSRWQQGNVFSCSVMHEALHNHYTQKSLSLSPGK | |
| | | 983 | 984 | 1019 | 986 |
| 31 | CD3B2030 | QVQLVQSGAEVKK | EIVLTQSPATLSASPG | EIVLTQSPATLSASPGERVTLSCSASSSVSYMNWYQQKPGQ | NA |
| | (CD3) | PGSSVKVSCKASG | ERVTLSCSASSSVSYM | APRRWIYDSSKLASGVPARFSGSGSGRDYTLTISSLEPEDF | |
| | | YTFTRSTMHWVKQ | NWYQQKPGQAPRRWIY | AVYYCQQWSRNPPTFGGGTKVEIKGGSEGKSSGSGSESKST | |
| | | APGQGLEWIGYIN | DSSKLASGVPARFSGS | GGSQVQLVQSGAEVKKPGSSVKVSCKASGYTFTRSTMHWVK | |
| | | PSSAYTNYNQKFQ | GSGRDYTLTISSLEPE | QAPGQGLEWIGYINPSSAYTNYNQKFQGRVTLTADKSTSTA | |
| | | GRVTLTADKSTST | DFAVYYCQQWSRNPPT | YMELSSLRSEDTAVYYCASPQVHYDYNGFPYWGQGTLVTVS | |
| | | AYMELSSLRSEDT | FGGGTKVEIK | SEPKSSDKTHTCPPCPAPEAAGGPSVFLFPPKPKDTLMISR | |
| | | AVYYCASPQVHYD | | TPEVTCVVVSVSHEDPEVKFNWYVDGVEVHNAKTKPREEQY | |
| | | YNGFPYWGQGTLV | | NSTYRVVSVLTVLHQDWLNGKEYKCKVSNKALPAPIEKTIS | |
| | | TVSS | | KAKGQPREPQVYTLPPSREEMTKNQVSLWCLVKGFYPSDIA | |
| | | | | VEWESNGQPENNYKTTPPVLDSDGSFFLYSKLTVDKSRWQQ | |
| | | | | GNVFSCSVMHEALHNHYTQKSLSLSPGK | |
| | | 1463 | 1464 | 1504 | 1466 |
| 32 | CD3B2030- | QVQLVQSGAEVKK | (see above) | EIVLTQSPATLSASPGERVTLSCSASSSVSYMNWYQQKPGQ | NA |
| | N106A | PGSSVKVSCKASG | | APRRWIYDSSKLASGVPARFSGSGSGRDYTLTISSLEPEDF | |
| | (CD3) | YTFTRSTMHWVKQ | | AVYYCQQWSRNPPTFGGGTKVEIKGGSEGKSSGSGSESKST | |
| | | APGQGLEWIGYIN | | GGSQVQLVQSGAEVKKPGSSVKVSCKASGYTFTRSTMHWVK | |
| | | PSSAYTNYNQKFQ | | QAPGQGLEWIGYINPSSAYTNYNQKFQGRVTLTADKSTSTA | |
| | | GRVTLTADKSTST | | YMELSSLRSEDTAVYYCASPQVHYDYAGFPYWGQGTLVTVS | |
| | | AYMELSSLRSEDT | | SEPKSSDKTHTCPPCPAPEAAGGPSVFLFPPKPKDTLMISR | |
| | | AVYYCASPQVHYD | | TPEVTCVVVSVSHEDPEVKFNWYVDGVEVHNAKTKPREEQY | |
| | | YAGFPYWGQGTLV | | NSTYRVVSVLTVLHQDWLNGKEYKCKVSNKALPAPIEKTIS | |
| | | TVSS | | KAKGQPREPQVYTLPPSREEMTKNQVSLWCLVKGFYPSDIA | |
| | | | | VEWESNGQPENNYKTTPPVLDSDGSFFLYSKLTVDKSRWQQ | |
| | | | | GNVFSCSVMHEALHNHYTQKSLSLSPGK | |
| | | 1505 | 1464 | 1455 | |

TABLE 17
CD3 Antibody Kabat CDR Amino Acid Sequences

| | Protein | HC Kabat | HC Kabat | HC Kabat | LC Kabat | LC Kabat | LC Kabat |
|---|---|---|---|---|---|---|---|
| # | Name | CDR1 | CDR2 | CDR3 | CDR1 | CDR2 | CDR3 |
| 25 | CD3B376+ | NNNAAWS | RTYYRSKWLYDYAVSVK | GYSSSFDY | TGTSSNIGTYKFVS | EVSKRPS | VSYAGSGTLL |
| | K477 | | S | | | | |
| | | 817 | 818 | 819 | 820 | 821 | 822 |
| 26 | CD3B376- | NNNAAWS | RTYYRSKWLYDYAVSVK | GYSSSFDY | TGTSSNIGTYKFVS | EVSKRPS | VSYAGSGTLL |
| | K477 | | S | | | | |
| | | 817 | 818 | 819 | 820 | 821 | 822 |
| 27 | CD3B450+ | NNNAAWS | RTYYRSKWLYDYAVSVK | GYSSSFDY | TGTSSNIGTYKFVS | EVSKRPS | VSYAGSGTLL |
| | K477 | | S | | | | |
| | | 817 | 818 | 819 | 820 | 821 | 822 |
| 28 | CD3B450- | NNNAAWS | RTYYRSKWLYDYAVSVK | GYSSSFDY | TGTSSNIGTYKFVS | EVSKRPS | VSYAGSGTLL |
| | K477 | | S | | | | |
| | | 817 | 818 | 819 | 820 | 821 | 822 |
| 29 | CD3W245+ | RYNMN | SISTSSNYIYYADSVKG | GWGPFDY | RARQSIGTAIH | YASESIS | QQSGSWPYT |
| | K477 | | | | | | |
| | | 1 | 954 | 955 | 956 | 957 | 958 |
| 30 | CD3W245- | RYNMN | SISTSSNYIYYADSVKG | GWGPFDY | RARQSIGTAIH | YASESIS | QQSGSWPYT |
| | K477 | | | | | | |
| | | 1 | 954 | 955 | 956 | 957 | 958 |
| 31 | CD3B2030 | RSTMH | YINPSSAYTNYNQKFQG | PQVHYDYNGFPY | SASSSVSYMN | DSSKLAS | QQWSRNPPT |
| | | 1467 | 1468 | 1469 | 1470 | 1471 | 1472 |
| 32 | CD3B2030- | RSTMH | YINPSSAYTNYNQKFQG | PQVHYDYAGFPY | SASSSVSYMN | DSSKLAS | QQWSRNPPT |
| | N106A | | | | | | |
| | | 1467 | 1468 | 1506 | 1470 | 1471 | 1472 |

TABLE 18
CD3 Antibody Chothia CDR Amino Acid Sequences

| | | HC | | HC | | LC | |
|---|---|---|---|---|---|---|---|
| | Pro- | Cho- | HC | Cho- | LC | Cho- | LC |
| | tein | thia | Chothia | thia | Chothia | thia | Chothia |
| # | Name | CDR1 | CDR2 | CDR3 | CDR1 | CDR2 | CDR3 |
| 25 | CD3B | GDSVF | YYRSKWL | YSSSFD | TSSNIGT | EVS | YAGSGTL |
| | 376 + | NNNA | | | YKF | | |
| | K477 | | | | | | |
| | | 823 | 824 | 825 | 826 | 487 | 828 |
| 26 | CD3B | GDSVF | YYRSKWL | YSSSFD | TSSNIGT | EVS | YAGSGTL |
| | 376 − | NNNA | | | YKF | | |
| | K477 | | | | | | |
| | | 823 | 824 | 825 | 826 | 487 | 828 |
| 27 | CD3B | GDSVF | YYRSKWL | YSSSFD | TSSNIGT | EVS | YAGSGTL |
| | 450 + | NNNA | | | YKF | | |
| | K477 | | | | | | |
| | | 823 | 824 | 825 | 826 | 487 | 828 |
| 28 | CD3B | GDSVF | YYRSKWL | YSSSFD | TSSNIGT | EVS | YAGSGTL |
| | 450 − | NNNA | | | YKF | | |
| | K477 | | | | | | |
| | | 823 | 824 | 825 | 826 | 487 | 828 |
| 29 | CD3W | GFTFS | STSSNY | GWGPFD | RQSIGTA | YAS | SGSWPY |
| | 245 + | RY | | | | | |
| | K477 | | | | | | |
| | | 7 | 960 | 961 | 962 | 963 | 964 |
| 30 | CD3W | GFTFS | STSSNY | GWGPFD | RQSIGTA | YAS | SGSWPY |
| | 245 − | RY | | | | | |
| | K477 | | | | | | |
| | | 7 | 960 | 961 | 962 | 963 | 964 |
| 31 | CD3B | GYTFT | NPSSAY | PQVHYD | SSSVSY | DSS | WSRNPP |
| | 2030 | RS | | YNGFP | | | |
| | | 1473 | 1474 | 1475 | 1476 | 1477 | 1478 |
| 32 | CD3B | GYTFT | NPSSAY | PQVHYD | SSSVSY | DSS | WSRNPP |
| | 2030 − | RS | | YAGFP | | | |
| | N106A | | | | | | |
| | | 1473 | 1474 | 1507 | 1476 | 1477 | 1478 |

TABLE 19
CD3 Antibody AbM CDR Amino Acid Sequences

| | | HC | HC | HC | LC | LC | LC |
|---|---|---|---|---|---|---|---|
| | Protein | AbM | AbM | AbM | AbM | AbM | AbM |
| # | Name | CDR1 | CDR2 | CDR3 | CDR1 | CDR2 | CDR3 |
| 25 | CD3B376 + | GDSV | RTYY | GYSS | TGTSS | EVSKRPS | VSYA |
| | K477 | FNNN | RSKW | SFDY | NIGTY | | GSGT |
| | | AAWS | LYD | | KFVS | | LL |
| | | 829 | 830 | 819 | 820 | 821 | 822 |
| 26 | CD3B376 − | GDSV | RTYY | GYSS | TGTSS | EVSKRPS | VSYA |
| | K477 | FNNN | RSKW | SFDY | NIGTY | | GSGT |
| | | AAWS | LYD | | KFVS | | LL |
| | | 829 | 830 | 819 | 820 | 821 | 822 |
| 27 | CD3B450 + | GDSV | RTYY | GYSS | TGTSS | EVSKRPS | VSYA |
| | K477 | FNNN | RSKW | SFDY | NIGTY | | GSGT |
| | | AAWS | LYD | | KFVS | | LL |
| | | 829 | 830 | 819 | 820 | 821 | 822 |
| 28 | CD3B450 − | GDSV | RTYY | GYSS | TGTSS | EVSKRPS | VSYA |
| | K477 | FNNN | RSKW | SFDY | NIGTY | | GSGT |
| | | AAWS | LYD | | KFVS | | LL |
| | | 829 | 830 | 819 | 820 | 821 | 822 |
| 29 | CD3W245 + | GFTF | SIST | GWGP | RARQS | YASESIS | QQSG |
| | K477 | SRYN | SSNY | FDY | IGTAI | | SWPY |
| | | MN | IY | | H | | T |
| | | 13 | 966 | 955 | 956 | 957 | 958 |
| 30 | CD3W245 − | GFTF | SIST | GWGP | RARQS | YASESIS | QQSG |
| | K477 | SRYN | SSNY | FDY | IGTAI | | SWPY |
| | | MN | IY | | H | | T |
| | | 13 | 966 | 955 | 956 | 957 | 958 |
| 31 | CD3B2030 | GYTF | YINP | PQVH | SASS | DSSKLAS | QQWS |
| | | TRST | SSAY | YDYN | SVSY | | RNPP |
| | | MH | TN | GFPY | MN | | T |
| | | 1508 | 1509 | 1469 | 1470 | 1471 | 1472 |
| 32 | CD3B2030 − | GYTF | YINP | PQVH | SASS | DSSKLAS | QQWS |
| | N106A | TRST | SSAY | YDYA | SVSY | | RNPP |
| | | MH | TN | GFPY | MN | | T |
| | | 1508 | 1509 | 1506 | 1470 | 1471 | 1472 |

TABLE 20
CD3 Antibody Contact CDR Amino Acid Sequences

| | | HC | HC | HC | LC | LC | LC |
|---|---|---|---|---|---|---|---|
| | | Con- | Con- | Con- | Con- | Con- | Con- |
| | Protein | tact | tact | tact | tact | tact | tact |
| # | Name | CDR1 | CDR2 | CDR3 | CDR1 | CDR2 | CDR3 |
| 25 | CD3B376 + | FNNN | WLGRT | ARGY | IGTY | VLLY | VSYA |
| | K477 | AAWS | YYRSK | SSSF | KFVS | EVSK | GSGT |
| | | | WLYD | D | WY | RP | L |
| | | 835 | 836 | 837 | 838 | 839 | 840 |
| 26 | CD3B376 − | FNNN | WLGRT | ARGY | IGTY | VLLY | VSYA |
| | K477 | AAWS | YYRSK | SSSF | KFVS | EVSK | GSGT |
| | | | WLYD | D | WY | RP | L |
| | | 835 | 836 | 837 | 838 | 839 | 840 |
| 27 | CD3B450 + | FNNN | WLGRT | ARGY | IGTY | VMIY | VSYA |
| | K477 | AAWS | YYRSK | SSSF | KFVS | EVSK | GSGT |
| | | | WLYD | D | WY | RP | L |
| | | 835 | 836 | 837 | 838 | 907 | 840 |
| 28 | CD3B450 − | FNNN | WLGRT | ARGY | IGTY | VMIY | VSYA |
| | K477 | AAWS | YYRSK | SSSF | KFVS | EVSK | GSGT |
| | | | WLYD | D | WY | RP | L |
| | | 835 | 836 | 837 | 838 | 907 | 840 |
| 29 | CD3W245 + | SRYN | WVSSI | TRGW | GTAI | LLIK | QQSG |
| | K477 | MN | STSSN | GPFD | HWY | YASE | SWPY |
| | | | YIY | | | SI | |
| | | 19 | 972 | 973 | 974 | 975 | 976 |
| 30 | CD3W245 − | SRYN | WVSSI | TRGW | GTAI | LLIK | QQSG |
| | K477 | MN | STSSN | GPFD | HWY | YASE | SWPY |
| | | | YIY | | | SI | |
| | | 19 | 972 | 973 | 974 | 975 | 976 |
| 31 | CD3B2030 | TRST | WIGYI | ASPQV | SYMN | RWIY | QQWS |
| | | MH | NPSSA | HYDYN | WY | DSSK | RNPP |
| | | | YTN | GFP | | LA | |
| | | 1510 | 1511 | 1512 | 1513 | 1514 | 1515 |
| 32 | CD3B2030 − | TRST | WIGYI | ASPQV | SYMN | RWIY | QQWS |
| | N106A | MH | NPSSA | HYDYA | WY | DSSK | RNPP |
| | | | YTN | GFP | | LA | |
| | | 1516 | 1511 | 1517 | 1513 | 1514 | 1515 |

TABLE 21
CD3 Antibody IMGT CDR Amino Acid Sequences

| | | HC | HC | HC | LC | LC | LC |
|---|---|---|---|---|---|---|---|
| | Protein | IMGT | IMGT | IMGT | IMGT | IMGT | IMGT |
| # | Name | CDR1 | CDR2 | CDR3 | CDR1 | CDR2 | CDR3 |
| 25 | CD3B376 + | GDSV | TYYR | ARGYS | SSNIG | EVS | VSYAG |
| | K477 | FNNN | SKWL | SSFDY | TYKF | | SGTLL |
| | | AA | Y | | | | |
| | | 841 | 842 | 843 | 844 | 487 | 822 |
| 26 | CD3B376 − | GDSV | TYYR | ARGYS | SSNIG | EVS | VSYAG |
| | K477 | FNNN | SKWL | SSFDY | TYKF | | SGTLL |
| | | AA | Y | | | | |
| | | 841 | 842 | 843 | 844 | 487 | 822 |
| 27 | CD3B450 + | GDSV | TYYR | ARGYS | SSNIG | EVS | VSYAG |
| | K477 | FNNN | SKWL | SSFDY | TYKF | | SGTLL |
| | | AA | Y | | | | |
| | | 841 | 842 | 843 | 844 | 487 | 822 |
| 28 | CD3B450 − | GDSV | TYYR | ARGYS | SSNIG | EVS | VSYAG |
| | K477 | FNNN | SKWL | SSFDY | TYKF | | SGTLL |
| | | AA | Y | | | | |
| | | 841 | 842 | 843 | 844 | 487 | 822 |
| 29 | CD3W245 + | GFTF | ISTS | TRGWG | QSIGT | YAS | QQSGS |
| | K477 | SRYN | SNYI | PFDY | A | | WPYT |
| | | 25 | 978 | 979 | 980 | 963 | 958 |
| 30 | CD3W245 − | GFTF | ISTS | TRGWG | QSIGT | YAS | QQSGS |
| | K477 | SRYN | SNYI | PFDY | A | | WPYT |
| | | 25 | 978 | 979 | 980 | 963 | 958 |
| 31 | CD3B2030 | GYTF | INPS | ASPQV | SSVSY | DSS | QQWSR |
| | | TRST | SAYT | HYDYN | | | NPPT |
| | | | | GFPY | | | |
| | | 1479 | 1480 | 1481 | 1482 | 1477 | 1472 |
| 32 | CD3B2030 − | GYTF | INPS | ASPQV | SSVSY | DSS | QQWSR |
| | N106A | TRST | SAYT | HYDYA | | | NPPT |
| | | | | GFPY | | | |
| | | 1479 | 1480 | 1518 | 1482 | 1477 | 1472 |

Exemplary CD3 VH and VL nucleic acid sequences are provided below.

VH nucleic acid sequence of CD3B2197 (CD3B376 + K477)
SEQ ID NO: 1453,
CAGGTGCAGCTGCAGCAGTCTGGCCCTAGACTCGTGCGGCCTTCCCAGACCCTGTCTCTGACCTGTG
CCATCTCCGGCGACTCCGTGTTCAACAACAACGCCGCCTGGTCCTGGATCCGGCAGTCTCCATCTCG
CGGTCTGGAGTGGCTCGGTCGCACCTACTACCGCTCTAAATGGCTGTACGACTACGCCGTGTCCGTG
AAGTCCCGGATCACCGTGAACCCTGACACCTCCCGGAACCAGTTCACCCTGCAGCTGAACTCCGTGA
CCCCTGAGGACACCGCCCTGTACTACTGCGCCAGAGGCTACTCCTCCTCCTTCGACTATTGGGGCCA
AGGCACCCTCGTGACCGTGTCCTCT
VL nucleic acid sequence of CD3B2197 (CD3B376 + K477)
SEQ ID NO: 1170,
CAGTCTGCTCTGACCCAGCCTGCCTCCGTGTCTGGCTCTCCCGGCCAGTCCATCACCATCAGCTGTA
CCGGCACCTCCTCCAACATCGGCACCTACAAGTTCGTGTCCTGGTATCAGCAACACCCCGACAAGGC
CCCCAAAGTGCTGCTGTACGAGGTGTCCAAGCGGCCCTCTGGCGTGTCCTCCAGATTCTCCGGCTCC
AAGTCTGGCAACACCGCCTCCCTGACCATCAGCGGACTGCAGGCTGAGGACCAGGCCGACTACCACT
GTGTGTCCTACGCTGGCTCTGGCACCCTGCTGTTTGGCGGAGGCACCAAGCTGACTGTCCTG
VH nucleic acid sequence of CD3B891 (CD3B376-K477)
SEQ ID NO: 1151,
CAGGTGCAGCTGCAGCAGTCTGGCCCTAGACTCGTGCGGCCTTCCCAGACCCTGTCTCTGACCTGTG
CCATCTCCGGCGACTCCGTGTTCAACAACAACGCCGCCTGGTCCTGGATCCGGCAGAGCCCTTCTAG
AGGCCTGGAATGGCTGGGCCGGACCTACTACCGGTCCAAGTGGCTGTACGACTACGCCGTGTCCGTG
AAGTCCCGGATCACCGTGAACCCTGACACCTCCCGGAACCAGTTCACCCTGCAGCTGAACTCCGTGA
CCCCTGAGGACACCGCCCTGTACTACTGCGCCAGAGGCTACTCCTCCTCCTTCGACTATTGGGGCCA
GGGCACCCTCGTGACCGTGTCCTCT
VL nucleic acid sequence of CD3B891 (CD3B376-K477)
SEQ ID NO: 1152,
CAGTCTGCTCTGACCCAGCCTGCCTCCGTGTCTGGCTCTCCCGGCCAGTCCATCACCATCAGCTGTA
CCGGCACCTCCTCCAACATCGGCACCTACAAGTTCGTGTCCTGGTATCAGCAGCACCCCGACAAGGC
CCCCAAAGTGCTGCTGTACGAGGTGTCCAAGCGGCCCTCTGGCGTGTCCTCCAGATTCTCCGGCTCC
AAGTCTGGCAACACCGCCTCCCTGACCATCAGCGGACTGCAGGCTGAGGACCAGGCCGACTACCACT
GTGTGTCCTACGCTGGCTCTGGCACCCTGCTGTTTGGCGGAGGCACCAAGCTGACCGTGCTG
VH nucleic acid sequence of CD3B2200 (CD3B450 + K477)
SEQ ID NO: 1153,
CAGGTTCAGCTGCAGCAGTCTGGCCCTGGACTGGTCAAGCCCTCTCAGACCCTGTCTCTGACCTGTG
CCATCTCCGGCGACTCCGTGTTCAACAACAATGCCGCCTGGTCCTGGATTCGGCAGTCTCCTAGTAG
AGGCCTGGAATGGCTGGGCAGAACCTACTACCGGTCCAAGTGGCTGTACGACTACGCCGTGTCCGTG
AAGTCCCGGATCACCATCAATCCCGACACCTCCAAGAACCAGTTCTCCCTGCAGCTCAACAGCGTGA
CCCCTGAGGATACCGCCGTGTACTACTGTGCCAGAGGCTACTCCTCCTCCTTCGACTATTGGGGCCA
GGGCACACTGGTCACCGTTTCTTCT
VL nucleic acid sequence of CD3B2200 (CD3B450 + K477)
SEQ ID NO: 1154,
CAGTCTGCTCTGACCCAGCCTGCTTCTGTGTCTGGCTCTCCCGGCCAGTCCATCACCATCTCTTGTA
CCGGCACCTCCTCCAACATCGGCACCTACAAGTTCGTGTCCTGGTATCAGCAGCACCCCGGCAAGGC
CCCTAAAGTGATGATCTACGAGGTGTCCAAGCGGCCCTCTGGCGTGTCCAACAGATTCTCCGGCTCC
AAGTCCGGCAATACCGCCTCTCTGACAATCAGCGGACTGCAGGCTGAGGACGAGGCCGACTACTACT
GTGTGTCTTACGCTGGCTCTGGCACCCTGTTGTTTGGCGGCGGAACAAAGCTGACTGTGCTG
VH nucleic acid sequence of CD3B2186 (CD3B450-K477)
SEQ ID NO: 1155,
CAGGTGCAGCTGCAGCAGAGCGGCCCCGGCCTGGTCAAGCCCAGCCAGACCCTGAGCCTGACCTGCG
CCATCAGCGGCGACAGCGTGTTCAACAACAACGCCGCCTGGTCCTGGATCCGCCAGAGCCCCAGCCG
CGGCCTGGAGTGGCTGGGCCGCACCTACTACCGCAGCAAGTGGCTGTACGACTACGCCGTGTCCGTG
AAGTCCCGCATCACCATCAACCCCGACACCAGCAAGAACCAGTTCTCCCTGCAGCTGAACAGCGTGA
CCCCCGAGGACACCGCCGTGTACTACTGCGCCCGCGGCTACAGCAGCAGCTTCGACTACTGGGGCCA
GGGCACCCTGGTCACCGTGTCCAGC
VL nucleic acid sequence of CD3B2186 (CD3B450-K477)
SEQ ID NO: 1156,
CAGTCTGCTCTGACCCAGCCTGCCTCCGTGTCTGGCTCTCCCGGCCAGTCCATCACCATCAGCTGTA
CCGGCACCTCCTCCAACATCGGCACCTACAAGTTCGTGTCCTGGTATCAGCAGCACCCCGGCAAGGC
CCCCAAAGTGATGATCTACGAGGTGTCCAAGCGGCCCTCCGGCGTGTCCAACAGATTCTCCGGCTCC
AAGTCCGGCAACACCGCCTCCCTGACAATCAGCGGACTGCAGGCCGAGGACGAGGCCGACTACTACT
GTGTGTCCTACGCCGGCTCTGGCACCCTGCTGTTTGGCGGCGGAACAAAGCTGACCGTGCTG
VH nucleic acid sequence of CD3B2183 (CD3W245)
SEQ ID NO: 1157,
GAGGTGCAACTGGTGGAGTCTGGGGGAGGCCTGGTCAAGCCTGGGGGGTCCCTGAGACTCTCCTGTG
CAGCCTCTGGATTCACCTTCAGTAGATATAACATGAACTGGGTCCGCCAGGCTCCAGGGAAGGGGCT
GGAGTGGGTCTCATCCATTAGTACTAGTAGTAATTACATATACTACGCAGACTCAGTGAAGGGCCGA
TTCACCTTCTCCAGAGACAACGCCAAGAACTCACTGGATCTGCAAATGAGCGGCCTGAGAGCCGAGG
ACACGGCTATTTATTACTGTACGAGAGGCTGGGGGCCTTTTGACTACTGGGGCCAGGGAACCCTGGT
CACCGTCTCCTCA
VL nucleic acid sequence of CD3B2183 (CD3W245)
SEQ ID NO: 1158,
GACATACAAATGACACAATCACCCTCTTCTCTTTCTGCAAGCGTTGGCGACCGTGTCACTATCACTT
GTCGAGCCCGCCAGTCCATAGGTACTGCCATTCACTGGTATCAACAGAAGCCTGGCAAGGCTCCCAA
ACTCCTGATTAAGTATGCCAGCGAGAGCATTTCCGGCGTACCTTCAAGATTTTCCGGCTCCGGTAGT
GGGACAGATTTCACTCTCACTATATCTAGCCTCCAACCAGAAGATTTCGCCACTTACTACTGTCAAC
AATCAGGTTCATGGCCTTACACTTTCGGCCAGGGGACAAAATTGGAGATCAAG
VH nucleic acid sequence of CD3B2030
SEQ ID NO: 1519,
CAGGTTCAGCTGGTTCAGTCTGGCGCCGAAGTGAAGAAACCTGGCTCCTCCGTCAAGGTGTCCTGCA
AGGCTTCCGGCTACACCTTTACCAGATCCACCATGCACTGGGTCAAACAGGCTCCAGGACAAGGCTT
GGAGTGGATCGGCTACATCAACCCCAGCTCCGCCTACACCAACTACAACCAGAAATTCCAGGGCAGA
GTCACCCTCACCGCCGACAAGTCTACCTCCACCGCCTACATGGAACTGTCCAGCCTGAGATCTGAGG
ACACCGCCGTGTACTACTGCGCCAGCCCTCAGGTGCACTACGACTACAACGGCTTCCCTTATTGGGG
CCAGGGCACCCTGGTTACCGTTTCTTCT
VL nucleic acid sequence of CD3B2030
SEQ ID NO: 1520,
GAGATCGTGCTGACCCAGTCTCCTGCCACACTGTCAGCCTCTCCAGGCGAGAGAGTCACCCTGTCCT
GCTCCGCTTCCTCCTCCGTGTCCTACATGAACTGGTATCAGCAGAAGCCCGGCCAGGCTCCTAGACG
GTGGATCTACGACTCCTCCAAGCTGGCCTCTGGCGTCCCTGCCCGCTTTTCCGGCTCTGGCTCTGGC
AGAGACTATACCCTGACCATCTCCAGCCTGGAACCTGAGGACTTCGCCGTGTACTACTGCCAGCAGT
GGTCTAGAAACCCTCCTACCTTTGGCGGAGGCACCAAGGTGGAAATCAAG
VH nucleic acid sequence of CD3B2030-N106A
SEQ ID NO: 1521,
CAGGTTCAACTGGTTCAGTCTGGCGCCGAAGTGAAGAAACCTGGCTCCTCCGTCAAGGTGTCCTGCA
AGGCTTCCGGCTACACCTTTACCAGATCCACCATGCACTGGGTCAAGCAGGCCCCTGGACAAGGCTT
GGAGTGGATCGGCTACATCAACCCCAGCTCCGCCTACACCAACTACAACCAGAAATTCCAGGGCAGA
GTGACCCTGACCGCCGACAAGTCTACCTCCACCGCCTACATGGAACTGTCCAGCCTGAGATCTGAGG
ACACCGCCGTGTACTACTGCGCCTCTCCTCAGGTCCACTACGACTACGCCGGCTTTCCTTATTGGGG
CCAGGGCACACTGGTCACCGTTTCTTCT
VL nucleic acid sequence of CD3B2030-N106A
SEQ ID NO: 1522,
GAGATCGTGCTGACCCAGTCTCCTGCCACACTGTCAGCCTCTCCAGGCGAGAGAGTCACCCTGTCCT
GCTCCGCTTCCTCCTCCGTGTCCTACATGAACTGGTATCAGCAGAAGCCCGGCCAGGCTCCTAGACG
GTGGATCTACGACTCCTCCAAGCTGGCCTCTGGCGTCCCTGCCCGCTTTTCCGGCTCTGGCTCTGGC
AGAGACTATACCCTGACCATCTCCAGCCTGGAACCTGAGGACTTCGCCGTGTACTACTGCCAGCAGT
GGTCTAGAAACCCTCCTACCTTTGGCGGAGGCACCAAGGTGGAAATCAAG

CD3 VH/VL regions were also engineered as scFvs in either VH-Linker-VL (also termed “HL”) or VL-linker-VH (also termed “LH”) orientations using the linker of SEQ ID NO:1419. The VH-Linker-VL or VL-linker-VH scFv molecules binding CD3 were further engineered into a scFv-hinge-CH2-CH3 (also termed scFv-Fc) format comprising Fc silencing mutation (L234A/L235A/D265S) and mutations designed to promote selective heterodimerization.

Exemplary scFv amino acid sequences of CD3W245 are provided below (LH or HL orientation). In some embodiments, PSMAxCD3 multispecific antibodies provided herein comprise an amino acid sequence of CD3W245-LH. In some embodiments, PSMAxCD3 multispecific antibodies provided herein comprise an amino acid sequence of CD3W245-HL.

CD3W245-LH
SEQ ID NO: 1186
DIQMTQSPSSLSASVGDRVTITCRARQSIGTAIHWYQQKPGKAPKLLIKY
ASESISGVPSRFSGSGSGTDFTLTISSLQPEDFATYYCQQSGSWPYTFGQ
GTKLEIKGGSEGKSSGSGSESKSTGGSEVQLVESGGGLVKPGGSLRLSCA
ASGFTFSRYNMNWVRQAPGKGLEWVSSISTSSNYIYYADSVKGRFTFSRD
NAKNSLDLQMSGLRAEDTAIYYCTRGWGPFDYWGQGTLVTVSS
CD3W245-HL
SEQ ID NO: 1187
EVQLVESGGGLVKPGGSLRLSCAASGFTFSRYNMNWVRQAPGKGLEWVSS
ISTSSNYIYYADSVKGRFTFSRDNAKNSLDLQMSGLRAEDTAIYYCTRGW
GPFDYWGQGTLVTVSSGGSEGKSSGSGSESKSTGGSDIQMTQSPSSLSAS
VGDRVTITCRARQSIGTAIHWYQQKPGKAPKLLIKYASESISGVPSRFSG
SGSGTDFTLTISSLQPEDFATYYCQQSGSWPYTFGQGTKLEIK

Exemplary scFv amino acid sequences of CD3B2030 and CD3B2030-N106A are provided below in the LH orientation. In some embodiments, PSMAxCD3 multispecific antibodies provided herein comprise an amino acid sequence of CD3B2030-LH. In some embodiments, PSMAxCD3 multispecific antibodies provided herein comprise an amino acid sequence of CD3B2030-N106A-LH.

CD3B2030-LH scFv amino acid sequence
SEQ ID NO: 1523,
EIVLTQSPATLSASPGERVTLSCSASSSVSYMNWYQQKPGQAPRRWIYDS
SKLASGVPARFSGSGSGRDYTLTISSLEPEDFAVYYCQQWSRNPPTFGGG
TKVEIKGGSEGKSSGSGSESKSTGGSQVQLVQSGAEVKKPGSSVKVSCKA
SGYTFTRSTMHWVKQAPGQGLEWIGYINPSSAYTNYNQKFQGRVTLTADK
STSTAYMELSSLRSEDTAVYYCASPQVHYDYNGFPYWGQGTLVTVSS
CD3B2030-N106A-LH scFv amino acid sequence
SEQ ID NO: 1524,
EIVLTQSPATLSASPGERVTLSCSASSSVSYMNWYQQKPGQAPRRWIYDS
SKLASGVPARFSGSGSGRDYTLTISSLEPEDFAVYYCQQWSRNPPTFGGG
TKVEIKGGSEGKSSGSGSESKSTGGSQVQLVQSGAEVKKPGSSVKVSCKA
SGYTFTRSTMHWVKQAPGQGLEWIGYINPSSAYTNYNQKFQGRVTLTADK
STSTAYMELSSLRSEDTAVYYCASPQVHYDYQGFPYWGQGTLVTVSS
CD3B2030_N106A-LH scFv nucleotide sequence
SEQ ID NO: 1525,
GAAATTGTTTTGACCCAATCACCTGCCACTCTCTCTGCCTCTCCTGGTGA
GCGAGTTACTTTGTCATGTAGCGCATCATCAAGTGTATCTTACATGAACT
GGTACCAACAAAAACCCGGACAGGCACCACGTCGTTGGATTTATGACAGT
AGCAAGCTCGCCTCCGGGGTACCCGCAAGATTTTCCGGGTCAGGGTCTGG
CAGGGACTATACCCTGACAATCAGCAGTCTGGAACCTGAGGACTTTGCTG
TGTATTACTGCCAACAGTGGTCTCGCAACCCCCCTACTTTCGGGGGAGGT
ACAAAGGTAGAAATTAAGGGCGGCTCCGAGGGCAAGAGCAGCGGCAGCGG
CAGCGAGAGCAAGAGCACCGGCGGCAGCCAAGTGCAACTCGTGCAAAGCG
GGGCTGAAGTGAAGAAGCCTGGATCAAGCGTGAAGGTCAGTTGCAAAGCC
TCTGGATATACCTTCACTCGATCAACCATGCACTGGGTCAAGCAGGCCCC
AGGGCAAGGGCTCGAATGGATAGGATATATTAACCCAAGTTCTGCCTACA
CTAACTATAATCAGAAGTTTCAAGGCCGGGTAACACTTACAGCCGATAAG
AGTACCTCAACAGCATACATGGAACTTAGTTCTTTGCGGAGCGAGGATAC
CGCTGTGTATTACTGCGCTTCACCTCAGGTTCACTACGACTACGCTGGAT
TTCCCTATTGGGGTCAGGGTACACTGGTTACAGTTTCCTCTG

In some embodiments, PSMAxCD3 multispecific antibodies provided herein comprise an amino acid sequence of SEQ ID NO:1186. In some embodiments, PSMAxCD3 multispecific antibodies provided herein comprise an amino acid sequence of SEQ ID NO:1187. In some embodiments, PSMAxCD3 multispecific antibodies provided herein comprise an amino acid sequence of SEQ ID NO:1523. In some embodiments, PSMAxCD3 multispecific antibodies provided herein comprise an amino acid sequence of SEQ ID NO:1524.

Other exemplary CD3 DuoBody HC and LC amino acid sequences are provided below.

HC amino acid sequence (DuoBody) of CD3B2183
(CD3W245)
SEQ ID NO: 1167,
EVQLVESGGGLVKPGGSLRLSCAASGFTFSRYNMNWVRQAPGKGLEWVSS
ISTSSNYIYYADSVKGRFTFSRDNAKNSLDLQMSGLRAEDTAIYYCTRGW
GPFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPEAAGGPSVFLFPPKPKDTL
MISRTPEVTCVVVSVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFLLYSKLTVDKSRWQQGNVFSCSVMHEALHNHYTQKSLSLSP

Exemplary CD3 DuoBody HC and LC nucleic acid sequences are provided below.

HC nucleic acid sequence (DuoBody) of CD3B2197
(CD3B376 + K477)
SEQ ID NO: 1149,
CAGGTGCAGCTGCAGCAGTCTGGCCCTAGACTCGTGCGGCCTTCCCAGACCCTGTCTCTGACCTGTG
CCATCTCCGGCGACTCCGTGTTCAACAACAACGCCGCCTGGTCCTGGATCCGGCAGTCTCCATCTCG
CGGTCTGGAGTGGCTCGGTCGCACCTACTACCGCTCTAAATGGCTGTACGACTACGCCGTGTCCGTG
AAGTCCCGGATCACCGTGAACCCTGACACCTCCCGGAACCAGTTCACCCTGCAGCTGAACTCCGTGA
CCCCTGAGGACACCGCCCTGTACTACTGCGCCAGAGGCTACTCCTCCTCCTTCGACTATTGGGGCCA
AGGCACCCTCGTGACCGTGTCCTCTGCCTCCACCAAGGGTCCATCGGTCTTCCCCCTGGCACCCTCC
TCCAAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGTG
ACAAAACTCACACTTGTCCACCGTGCCCAGCACCTGAAGCAGCAGGGGGACCGTCAGTCTTCCTCTT
CCCCCCAAAACCCAAGGACACCCTCATGATCTCCCGGACCCCTGAGGTCACATGCGTGGTGGTGAGC
GTGAGCCACGAAGACCCTGAGGTCAAGTTCAACTGGTACGTGGACGGCGTGGAGGTGCATAATGCCA
AGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTGTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGAC
TCCGACGGCTCCTTCCTGCTCTACAGCAAGCTCACCGTGGACAAGTCCAGATGGCAGCAGGGGAACG
TCTTCTCATGCTCCGTGATGCATGAGGCTCTGCACAACCACTACACGCAGAAGTCTCTCTCCCTGTC
TCCGGGAAAA
LC nucleic acid sequence (DuoBody) of CD3B2197
(CD3B376 + K477)
SEQ ID NO: 1150,
CAGTCTGCTCTGACCCAGCCTGCCTCCGTGTCTGGCTCTCCCGGCCAGTCCATCACCATCAGCTGTA
CCGGCACCTCCTCCAACATCGGCACCTACAAGTTCGTGTCCTGGTATCAGCAACACCCCGACAAGGC
CCCCAAAGTGCTGCTGTACGAGGTGTCCAAGCGGCCCTCTGGCGTGTCCTCCAGATTCTCCGGCTCC
AAGTCTGGCAACACCGCCTCCCTGACCATCAGCGGACTGCAGGCTGAGGACCAGGCCGACTACCACT
GTGTGTCCTACGCTGGCTCTGGCACCCTGCTGTTTGGCGGAGGCACCAAGCTGACTGTCCTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTCGAAACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
HC nucleic acid sequence (DuoBody) of CD3B891
(CD3B376-K477)
SEQ ID NO: 1171,
CAGGTGCAGCTGCAGCAGTCTGGCCCTAGACTCGTGCGGCCTTCCCAGACCCTGTCTCTGACCTGTG
CCATCTCCGGCGACTCCGTGTTCAACAACAACGCCGCCTGGTCCTGGATCCGGCAGAGCCCTTCTAG
AGGCCTGGAATGGCTGGGCCGGACCTACTACCGGTCCAAGTGGCTGTACGACTACGCCGTGTCCGTG
AAGTCCCGGATCACCGTGAACCCTGACACCTCCCGGAACCAGTTCACCCTGCAGCTGAACTCCGTGA
CCCCTGAGGACACCGCCCTGTACTACTGCGCCAGAGGCTACTCCTCCTCCTTCGACTATTGGGGCCA
GGGCACCCTCGTGACCGTGTCCTCTGCTTCCACCAAGGGCCCATCCGTCTTCCCCCTGGCACCCTCC
TCCAAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGTG
ACAAAACTCACACATGTCCACCGTGCCCAGCACCTGAAGCAGCAGGGGGACCGTCAGTCTTCCTCTT
CCCCCCAAAACCCAAGGACACCCTCATGATCTCCCGGACCCCTGAGGTCACATGCGTGGTGGTGAGC
GTGAGCCACGAAGACCCTGAGGTCAAGTTCAACTGGTACGTGGACGGCGTGGAGGTGCATAATGCCA
AGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGAC
TCCGACGGCTCCTTCCTGCTCTACAGCAAGCTCACCGTGGACAAGTCTAGATGGCAGCAGGGGAACG
TCTTCTCATGCTCCGTGATGCATGAGGCTCTGCACAACCACTACACGCAGAAGAGCCTCTCCCTGTC
TCCGGGT
LC nucleic acid sequence (DuoBody) of CD3B891
(CD3B376-K477)
SEQ ID NO: 1172,
CAGTCTGCTCTGACCCAGCCTGCCTCCGTGTCTGGCTCTCCCGGCCAGTCCATCACCATCAGCTGTA
CCGGCACCTCCTCCAACATCGGCACCTACAAGTTCGTGTCCTGGTATCAGCAGCACCCCGACAAGGC
CCCCAAAGTGCTGCTGTACGAGGTGTCCAAGCGGCCCTCTGGCGTGTCCTCCAGATTCTCCGGCTCC
AAGTCTGGCAACACCGCCTCCCTGACCATCAGCGGACTGCAGGCTGAGGACCAGGCCGACTACCACT
GTGTGTCCTACGCTGGCTCTGGCACCCTGCTGTTTGGCGGAGGCACCAAGCTGACCGTGCTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
HC nucleic acid sequence (DuoBody) of CD3B2200
(CD3B450 + K477)
SEQ ID NO: 1173,
CAGGTTCAGCTGCAGCAGTCTGGCCCTGGACTGGTCAAGCCCTCTCAGACCCTGTCTCTGACCTGTG
CCATCTCCGGCGACTCCGTGTTCAACAACAATGCCGCCTGGTCCTGGATTCGGCAGTCTCCTAGTAG
AGGCCTGGAATGGCTGGGCAGAACCTACTACCGGTCCAAGTGGCTGTACGACTACGCCGTGTCCGTG
AAGTCCCGGATCACCATCAATCCCGACACCTCCAAGAACCAGTTCTCCCTGCAGCTCAACAGCGTGA
CCCCTGAGGATACCGCCGTGTACTACTGTGCCAGAGGCTACTCCTCCTCCTTCGACTATTGGGGCCA
GGGCACACTGGTCACCGTTTCTTCTGCCTCCACCAAGGGTCCATCGGTCTTCCCCCTGGCACCCTCC
TCCAAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGTG
ACAAAACTCACACTTGTCCACCGTGCCCAGCACCTGAAGCAGCAGGGGGACCGTCAGTCTTCCTCTT
CCCCCCAAAACCCAAGGACACCCTCATGATCTCCCGGACCCCTGAGGTCACATGCGTGGTGGTGAGC
GTGAGCCACGAAGACCCTGAGGTCAAGTTCAACTGGTACGTGGACGGCGTGGAGGTGCATAATGCCA
AGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTGTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGAC
TCCGACGGCTCCTTCCTGCTCTACAGCAAGCTCACCGTGGACAAGTCCAGATGGCAGCAGGGGAACG
TCTTCTCATGCTCCGTGATGCATGAGGCTCTGCACAACCACTACACGCAGAAGTCTCTCTCCCTGTC
TCCGGGAAAA
LC nucleic acid sequence (DuoBody) of CD3B2200
(CD3B450 + K477)
SEQ ID NO: 1174,
CAGTCTGCTCTGACCCAGCCTGCTTCTGTGTCTGGCTCTCCCGGCCAGTCCATCACCATCTCTTGTA
CCGGCACCTCCTCCAACATCGGCACCTACAAGTTCGTGTCCTGGTATCAGCAGCACCCCGGCAAGGC
CCCTAAAGTGATGATCTACGAGGTGTCCAAGCGGCCCTCTGGCGTGTCCAACAGATTCTCCGGCTCC
AAGTCCGGCAATACCGCCTCTCTGACAATCAGCGGACTGCAGGCTGAGGACGAGGCCGACTACTACT
GTGTGTCTTACGCTGGCTCTGGCACCCTGTTGTTTGGCGGCGGAACAAAGCTGACTGTGCTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTCGAAACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
HC nucleic acid sequence (DuoBody) of CD3B2186
(CD3B450-K477)
SEQ ID NO: 1175,
CAGGTGCAGCTGCAGCAGAGCGGCCCCGGCCTGGTCAAGCCCAGCCAGACCCTGAGCCTGACCTGCG
CCATCAGCGGCGACAGCGTGTTCAACAACAACGCCGCCTGGTCCTGGATCCGCCAGAGCCCCAGCCG
CGGCCTGGAGTGGCTGGGCCGCACCTACTACCGCAGCAAGTGGCTGTACGACTACGCCGTGTCCGTG
AAGTCCCGCATCACCATCAACCCCGACACCAGCAAGAACCAGTTCTCCCTGCAGCTGAACAGCGTGA
CCCCCGAGGACACCGCCGTGTACTACTGCGCCCGCGGCTACAGCAGCAGCTTCGACTACTGGGGCCA
GGGCACCCTGGTCACCGTGTCCAGCGCTTCCACCAAGGGCCCATCCGTCTTCCCCCTGGCACCCTCC
TCCAAGAGCACCTCTGGGGGCACAGCGGCCCTGGGCTGCCTGGTCAAGGACTACTTCCCCGAACCGG
TGACGGTGTCGTGGAACTCAGGCGCCCTGACCAGCGGCGTGCACACCTTCCCGGCTGTCCTACAGTC
CTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGTG
ACAAAACTCACACATGTCCACCGTGCCCAGCACCTGAAGCAGCAGGGGGACCGTCAGTCTTCCTCTT
CCCCCCAAAACCCAAGGACACCCTCATGATCTCCCGGACCCCTGAGGTCACATGCGTGGTGGTGAGC
GTGAGCCACGAAGACCCTGAGGTCAAGTTCAACTGGTACGTGGACGGCGTGGAGGTGCATAATGCCA
AGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTACACCCTGCCCCCATCCC
GGGAGGAGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGAC
TCCGACGGCTCCTTCCTGCTCTACAGCAAGCTCACCGTGGACAAGTCTAGATGGCAGCAGGGGAACG
TCTTCTCATGCTCCGTGATGCATGAGGCTCTGCACAACCACTACACGCAGAAGAGCCTCTCCCTGTC
TCCGGGT
LC nucleic acid sequence (DuoBody) of CD3B2186
(CD3B450-K477)
SEQ ID NO: 1176,
CAGTCTGCTCTGACCCAGCCTGCCTCCGTGTCTGGCTCTCCCGGCCAGTCCATCACCATCAGCTGTA
CCGGCACCTCCTCCAACATCGGCACCTACAAGTTCGTGTCCTGGTATCAGCAGCACCCCGGCAAGGC
CCCCAAAGTGATGATCTACGAGGTGTCCAAGCGGCCCTCCGGCGTGTCCAACAGATTCTCCGGCTCC
AAGTCCGGCAACACCGCCTCCCTGACAATCAGCGGACTGCAGGCCGAGGACGAGGCCGACTACTACT
GTGTGTCCTACGCCGGCTCTGGCACCCTGCTGTTTGGCGGCGGAACAAAGCTGACCGTGCTGGGTCA
GCCCAAGGCTGCACCCAGTGTCACTCTGTTCCCGCCCTCCTCTGAGGAGCTTCAAGCCAACAAGGCC
ACACTGGTGTGTCTCATAAGTGACTTCTACCCGGGAGCCGTGACAGTGGCCTGGAAGGCCGATAGCA
GCCCCGTCAAGGCGGGAGTGGAGACCACCACACCCTCCAAACAAAGCAACAACAAGTACGCGGCCAG
CAGCTATCTGAGCCTGACGCCTGAGCAGTGGAAGTCCCACAGAAGCTACAGCTGCCAGGTCACGCAT
GAAGGGAGCACCGTGGAGAAGACAGTGGCCCCTACAGAATGTTCA
HC nucleic acid sequence (DuoBody) of CD3B2183
(CD3W245)
SEQ ID NO: 1177,
GAGGTGCAACTGGTGGAGTCTGGGGGAGGCCTGGTCAAGCCTGGGGGGTCCCTGAGACTCTCCTGTG
CAGCCTCTGGATTCACCTTCAGTAGATATAACATGAACTGGGTCCGCCAGGCTCCAGGGAAGGGGCT
GGAGTGGGTCTCATCCATTAGTACTAGTAGTAATTACATATACTACGCAGACTCAGTGAAGGGCCGA
TTCACCTTCTCCAGAGACAACGCCAAGAACTCACTGGATCTGCAAATGAGCGGCCTGAGAGCCGAGG
ACACGGCTATTTATTACTGTACGAGAGGCTGGGGGCCTTTTGACTACTGGGGCCAGGGAACCCTGGT
CACCGTCTCCTCAGCTTCCACCAAGGGCCCATCCGTCTTCCCCCTGGCACCCTCCTCCAAGAGCACC
TCTGGGGGCACAGCGGCCCTGGGCTGCCTGGTCAAGGACTACTTCCCCGAACCGGTGACGGTGTCGT
GGAACTCAGGCGCCCTGACCAGCGGCGTGCACACCTTCCCGGCTGTCCTACAGTCCTCAGGACTCTA
CTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAAAGTTGAGCCCAAATCTTGTGACAAAACTCACA
CATGTCCACCGTGCCCAGCACCTGAAGCAGCAGGGGGACCGTCAGTCTTCCTCTTCCCCCCAAAACC
CAAGGACACCCTCATGATCTCCCGGACCCCTGAGGTCACATGCGTGGTGGTGAGCGTGAGCCACGAA
GACCCTGAGGTCAAGTTCAACTGGTACGTGGACGGCGTGGAGGTGCATAATGCCAAGACAAAGCCGC
GGGAGGAGCAGTACAACAGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCCATCCCGGGAGGAGATGA
CCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCC
TTCCTGCTCTACAGCAAGCTCACCGTGGACAAGTCTAGATGGCAGCAGGGGAACGTCTTCTCATGCT
CCGTGATGCATGAGGCTCTGCACAACCACTACACGCAGAAGAGCCTCTCCCTGTCTCCGGGT
LC nucleic acid sequence (DuoBody) of CD3B2183
(CD3W245)
SEQ ID NO: 1178,
GACATACAAATGACACAATCACCCTCTTCTCTTTCTGCAAGCGTTGGCGACCGTGTCACTATCACTT
GTCGAGCCCGCCAGTCCATAGGTACTGCCATTCACTGGTATCAACAGAAGCCTGGCAAGGCTCCCAA
ACTCCTGATTAAGTATGCCAGCGAGAGCATTTCCGGCGTACCTTCAAGATTTTCCGGCTCCGGTAGT
GGGACAGATTTCACTCTCACTATATCTAGCCTCCAACCAGAAGATTTCGCCACTTACTACTGTCAAC
AATCAGGTTCATGGCCTTACACTTTCGGCCAGGGGACAAAATTGGAGATCAAGCGGACAGTGGCCGC
TCCTTCCGTGTTCATCTTCCCACCTTCCGACGAGCAGCTGAAGTCCGGCACAGCTTCTGTCGTGTGC
CTGCTGAACAACTTCTACCCTCGGGAAGCCAAGGTGCAGTGGAAGGTGGACAATGCCCTGCAGTCCG
GCAACTCCCAAGAGTCTGTGACCGAGCAGGACTCCAAGGACAGCACCTACAGCCTGTCCTCCACACT
GACCCTGTCCAAGGCCGACTACGAGAAGCACAAGGTGTACGCCTGCGAAGTGACCCATCAGGGCCTG
TCTAGCCCTGTGACCAAGTCTTTCAACCGGGGCGAGTGT

### Example 5: Generation of Bispecific PSMAxCD3 Antibodies

### Example 5.1: Fab-Arm Exchange of Anti-PSMA and Anti-CD3 Antibodies

The formation of bispecific antibodies requires two parental monoclonal antibodies (mAbs), one specific for the targeting arm (e.g. PSMA) and one specific for the effector arm (e.g. CD3). Selected monospecific anti-PSMA and anti-CD3 antibodies were expressed as IgG1/κ engineered to have L234A, L235A and D265S substitutions for cF silencing, (EU numbering). Selected monospecific anti-PSMA and anti-CD3 antibodies are also expressed as IgG4 antibodies. Mutations designed to promote selective heterodimerization of the Fc domain were also engineered in the Fc domain. In some cases, the anti-CD3 and anti-PSMA binders were formatted as scFv or Fab to test their stability in both formats. In other cases, scFv-based binders were tested in a VH-Linker-VL (HL) orientation or in a VL-Linker-VH orientation (LH).

The monospecific antibodies were expressed in CHO cell lines under CMV promoters as described above). The parental PSMA and CD3 antibodies were purified using a protein A column with an elution buffer of 100 mM NaAc pH 3.5 and a neutralization puffer of 2 M Tris pH 7.5 and 150 mM NaCl. The anti-PSMA and anti-CD3 monoclonal antibodies were dialyzed into D-PBS, pH 7.2 buffer.

For DuoBody antibodies, post purification of parental monospecific antibodies, bispecific PSMAxCD3 antibodies were generated by mixing the parental PSMA antibodies with the desired parental CD3 antibody under reducing conditions in 75 mM cysteamine-HCl and incubated overnight at room temperature for in vitro Fab arm exchange as described in Int. Patent Publ. No. WO2011/131746. The recombination reactions were based on molar ratios, where a set amount of PSMA antibody (e.g., 10 mg, or −74.6 nanomoles) was combined with CD3 antibody (e.g., −70.87 nanomoles), where the PSMA antibody was added in a 5% excess of the CD3 antibody. The concentrations of the PSMA antibody stocks varied from 0.8 to 6 mg/mL, and the volumes the recombination reactions varied for each pairing. The recombinations were subsequently dialyzed overnight against PBS to remove the reductant. The PSMAxCD3 bispecific antibody reactions were performed with an excess of the PSMA antibody (ratio) to minimize the amount of unreacted CD3 parental antibody remaining after recombination.

Other bispecifics were generated via co-transfection of HC1:HC2:LC2, typically at a DNA ratio of 1:1:3. Purification was performed by protein A chromatography and CH1 affinity capture, followed by an ion exchange-based chromatography.

Exemplary PSMAxCD3 multispecific antibodies are provided in Tables 22-27.

TABLE 22
PSMA × CD3 Bispecific Antibodies: Clone Descriptions

| Name | Bispecific Description |
|---|---|
| PS3B1353 | HC1 (F405L): CD3B376 x HC2 (K409R): PSMB947 |
| PS3B1505 | HC1 CD3B376-Fab x HC2 PSMB896-G100A IgGI DuoBody |
| PS3B1508 | HC1 (Knob3): CD3W245-LH-scFv; HC2 (Hole3-RF) PSMB896-G100A-Fab-RF: IgG1 AAS |
| PS3B1917 | HC1 (ZWA w/o K447_RF): CD3B376-Fab, HC2 (ZWB w/o K447): PSMA_P72_A10-HC-G54E-cFv LH |
| PS3B1918 | HC1 (ZWA w/o K447_RF): CD3B376-Fab, HC2 (ZWB w/o K447): PSMA_P72_D01-HC-D95E-scFv LH |
| PS3B1919 | HC1 (ZWA w/o K447_RF): CD3B376-Fab; HC2 (ZWB w/o K447): PSMA_P75_F01, LH |
| PS3B1920 | HC1 (ZWA w/o K447_RF): CD3B376-Fab; HC2 (ZWB w/o K447): PSMA_P72_F07, LH |
| PS3B1921 | HC1 (ZWA w/o K447_RF): CD3B376-Fab; HC2 (ZWB w/o K447): PSMA_P72_E07, LH |
| PS3B1922 | HC1 (ZWA w/o K447_RF): CD3B376-Fab; HC2 (ZWB w/o K447): PSMA_P72_D01, LH |
| PS3B1923 | HC1 (ZWA w/o K447_RF): CD3B376-Fab; HC2 (ZWB w/o K447): PSMA_P72_C01, LH |
| PS3B1924 | HC1 (ZWA w/o K447_RF): CD3B376-Fab; HC2 (ZWB w/o K447): PSMA_P72_A10, LH |
| PS3B1925 | HC1 (ZWA w/o K447_RF): CD3B376-Fab; HC2 (ZWB w/o K447): PSMA_P70_F02, LH |
| PS3B1926 | HC1 (ZWA w/o K447_RF): CD3B376-Fab; HC2 (ZWB w/o K447): PSMA_P72_G02, HL |
| PS3B1927 | HC1 (ZWA w/o K447_RF): CD3B376-Fab; HC2 (ZWB w/o K447): PSMA_P72_C01, HL |
| PS3B1928 | HC1 (ZWA w/o K447_RF): CD3B376-Fab; HC2 (ZWB w/o K447): PSMA_P72_A11, HL |
| PSMB1041 | HC1 (ZWA): B23B62-Fab; HC2 (ZWB): PSMA_P70_F02, LH |
| PSMB1045 | HC1 (ZWA): B23B62-Fab; HC2 (ZWB): PSMA_P72_A10, LH |
| PSMB1047 | HC1 (ZWA): B23B62-Fab; HC2 (ZWB): PSMA_P72_C01, LH |
| PSMB1049 | HC1 (ZWA): B23B62-Fab; HC2 (ZWB): PSMA_P72_D01, LH |
| PSMB1051 | HC1 (ZWA): B23B62-Fab; HC2 (ZWB): PSMA_P72_E07, LH |
| PSMB1052 | HC1 (ZWA): B23B62-Fab; HC2 (ZWB): PSMA_P72_F07, LH |
| PSMB1060 | HC1 (ZWA): B23B62-Fab; HC2 (ZWB): PSMA_P75_F01, LH |
| PSMB1068 | HC1 (ZWA): B23B62-Fab; HC2 (ZWB): PSMA_P72_A11, HL |
| PSMB1069 | HC1 (ZWA): B23B62-Fab; HC2 (ZWB): PSMA_P72_C01, HL |
| PSMB1075 | HC1 (ZWA): B23B62-Fab; HC2 (ZWB): PSMA_P72_G02, HL |
| PSMB2908 | HC1 (ZWA w/o K447): B23B62-Fab, HC2 (ZWB w/o K447): PSMA_P72_D01-HC-D95E-scFv LH |
| PSMB2909 | HC1 (ZWA w/o K447): B23B62-Fab, HC2 (ZWB w/o K447)): PSMA_P72_A10-HC-G54E-scFv LH |
| PS3B1391 | HC1: N-term scFv LH CD3B2030 N106A LH-scFv (MA), HC2: N-term_Fab_PSMHB49SC1133_011A11_1 |
| PS3B1396 | HC1 (Knob3): CD3B2030-N106A-scFv LH, HC2 (Hole3): PSMB896-G100A-Fab |

TABLE 23
PSMA x CD3 Bispecific Antibodies: CD3 Arm Descriptions

| | | SEQ | | SEQ | CD3 Arm |
|---|---|---|---|---|---|
| | | ID | | ID | Descrip- |
| Name | HC1 | NO. | LC1 | NO. | tion |
| PS3B1353 | (see above) | 849 | (see above) | 850 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1505 | (see above) | 849 | (see above) | 850 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1508 | DIQMTQSPSSLSASVGDRVTITCRARQSIGTAIHWYQQKPGKAPK | 1192 | NA | 1193 | with |
| | LLIKYASESISGVPSRFSGSGSGTDFTLTISSLQPEDFATYYCQQ | | | | CD3W245 |
| | SGSWPYTFGQGTKLEIKGGSEGKSSGSGSESKSTGGSEVQLVESG | | | | arm |
| | GGLVKPGGSLRLSCAASGFTFSRYNMNWVRQAPGKGLEWVSSIST | | | | (CD3B2183 |
| | SSNYIYYADSVKGRFTFSRDNAKNSLDLQMSGLRAEDTAIYYCTR | | | | without |
| | GWGPFDYWGQGTLVTVSSEPKSSDKTHTCPPCPAPEAAGGPSVFL | | | | K477) |
| | FPPKPKDTLMISRTPEVTCVVVSVSHEDPEVKFNWYVDGVEVHNA | | | | |
| | KTKPREEQYNSTYRVVSVLTVLHQDWLNGKEYKCKVSNKALPAPI | | | | |
| | EKTISKAKGQPREPQVYTLPPSREEMTKNQVSLWCLVKGFYPSDI | | | | |
| | AVEWESNGQPENNYKTTPPVLDSDGSFFLYSKLTVDKSRWQQGNV | | | | |
| | FSCSVMHEALHNHYTQKSLSLSPGK | | | | |
| PS3B1917 | QVQLQQSGPRLVRPSQTLSLTCAISGDSVFNNNAAWSWIRQSPSR | 1194 | QSALTQPASVSGSPGQSIT | 1195 | with |
| | GLEWLGRTYYRSKWLYDYAVSVKSRITVNPDTSRNQFTLQLNSVT | | ISCTGTSSNIGTYKFVSWY | | CD3B376 |
| | PEDTALYYCARGYSSSFDYWGQGTLVTVSSASTKGPSVFPLAPSS | | QQHPDKAPKVLLYEVSKRP | | arm |
| | KSTSGGTAALGCLVKDYFPEPVTVSWNSGALTSGVHTFPAVLQSS | | SGVSSRFSGSKSGNTASLT | | (CD3B891 |
| | GLYSLSSVVTVPSSSLGTQTYICNVNHKPSNTKVDKKVEPKSCDK | | ISGLQAEDQADYHCVSYAG | | without |
| | THTCPPCPAPEAAGGPSVFLFPPKPKDTLMISRTPEVTCVVVSVS | | SGTLLFGGGTKLTVLRTVA | | K477 or |
| | HEDPEVKFNWYVDGVEVHNAKTKPREEQYNSTYRVVSVLTVLHQD | | APSVFIFPPSDEQLKSGTA | | CD3B2197 |
| | WLNGKEYKCKVSNKALPAPIEKTISKAKGQPREPQVYVYPPSREE | | SVVCLLNNFYPREAKVQWK | | with |
| | MTKNQVSLTCLVKGFYPSDIAVEWESNGQPENNYKTTPPVLDSDG | | VDNALQSGNSQESVTEQDS | | K477 |
| | SFALVSKLTVDKSRWQQGNVFSCSVMHEALHNRFTQKSLSLSPG | | KDSTYSLSSTLTLSKADYE | | in HC1) |
| | | | KHKVYACEVTHQGLSSPVT | | |
| | | | KSFNRGEC | | |
| PS3B1918 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1919 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1920 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1921 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1922 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1923 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1924 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1925 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1926 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1927 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PS3B1928 | (see above) | 1194 | (see above) | 1195 | with |
| | | | | | CD3B376 |
| | | | | | arm |
| | | | | | (CD3B891 |
| | | | | | without |
| | | | | | K477 or |
| | | | | | CD3B2197 |
| | | | | | with |
| | | | | | K477 |
| | | | | | in HC1) |
| PSMB1041 | QITLKESGPTLVKPTQTLTLTCTFSGFSLSTSGMGVSWIRQPPGK | 1218 | DIVMTQSPDSLAVSLGERA | 1219 | Null CD3 |
| | ALEWLAHIYWDDDKRYNPSLKSRLTITKDTSKNQVVLTMTNMDPV | | TINCRASQSVDYNGISYMH | | arm |
| | DTATYYCARLYGFTYGFAYWGQGTLVTVSSASTKGPSVFPLAPSS | | WYQQKPGQPPKLLIYAASN | | |
| | KSTSGGTAALGCLVKDYFPEPVTVSWNSGALTSGVHTFPAVLQSS | | PESGVPDRFSGSGSGTDFT | | |
| | GLYSLSSVVTVPSSSLGTQTYICNVNHKPSNTKVDKKVEPKSCDK | | LTISSLQAEDVAVYYCQQI | | |
| | THTCPPCPAPELLGGPSVFLFPPKPKDTLMISRTPEVTCVVVDVS | | IEDPWTFGQGTKVEIKRTV | | |
| | HEDPEVKFNWYVDGVEVHNAKTKPREEQYNSTYRVVSVLTVLHQD | | AAPSVFIFPPSDEQLKSGT | | |
| | WLNGKEYKCKVSNKALPAPIEKTISKAKGQPREPQVYVYPPSREE | | ASVVCLLNNFYPREAKVQW | | |
| | MTKNQVSLTCLVKGFYPSDIAVEWESNGQPENNYKTTPPVLDSDG | | KVDNALQSGNSQESVTEQD | | |
| | SFALVSKLTVDKSRWQQGNVFSCSVMHEALHNRFTQKSLSLSPG | | SKDSTYSLSSTLTLSKADY | | |
| | | | EKHKVYACEVTHQGLSSPV | | |
| | | | TKSFNRGEC | | |
| PSMB1045 | (see above) | 1218 | (see above) | 1219 | Null CD3 |
| | | | | | arm |
| PSMB1047 | (see above) | 1218 | (see above) | 1219 | Null CD3 |
| | | | | | arm |
| PSMB1049 | (see above) | 1218 | (see above) | 1219 | Null CD3 |
| | | | | | arm |
| PSMB1051 | (see above) | 1218 | (see above) | 1219 | Null CD3 |
| | | | | | arm |
| PSMB1052 | (see above) | 1218 | (see above) | 1219 | Null CD3 |
| | | | | | arm |
| PSMB1060 | (see above) | 1218 | (see above) | 1219 | Null CD3 |
| | | | | | arm |
| PSMB1068 | (see above) | 1218 | (see above) | 1219 | Null CD3 |
| | | | | | arm |
| PSMB1069 | (see above) | 1218 | (see above) | 1219 | Null CD3 |
| | | | | | arm |
| PSMB1075 | (see above) | 1218 | (see above) | 1219 | Null CD3 |
| | | | | | arm |
| PSMB2908 | QITLKESGPTLVKPTQTLTLTCTFSGFSLSTSGMGVSWIRQPPGK | 1238 | (see above) | 1219 | Null CD3 |
| | ALEWLAHIYWDDDKRYNPSLKSRLTITKDTSKNQVVLTMTNMDPV | | | | arm |
| | DTATYYCARLYGFTYGFAYWGQGTLVTVSSASTKGPSVFPLAPSS | | | | |
| | KSTSGGTAALGCLVKDYFPEPVTVSWNSGALTSGVHTFPAVLQSS | | | | |
| | GLYSLSSVVTVPSSSLGTQTYICNVNHKPSNTKVDKKVEPKSCDK | | | | |
| | THTCPPCPAPEAAGGPSVFLFPPKPKDTLMISRTPEVTCVVVSVS | | | | |
| | HEDPEVKFNWYVDGVEVHNAKTKPREEQYNSTYRVVSVLTVLHQD | | | | |
| | WLNGKEYKCKVSNKALPAPIEKTISKAKGQPREPQVYVYPPSREE | | | | |
| | MTKNQVSLTCLVKGFYPSDIAVEWESNGQPENNYKTTPPVLDSDG | | | | |
| | SFALVSKLTVDKSRWQQGNVFSCSVMHEALHNHYTQKSLSLSPG | | | | |
| PSMB2909 | (see above) | 1238 | (see above) | 1219 | Null CD3 |
| | | | | | arm |
| PS3B1391 | EIVLTQSPATLSASPGERVTLSCSASSSVSYMNWYQQKPGQAPRR | 1504 | NA | 1456 | CD3B2030 |
| | WIYDSSKLASGVPARFSGSGSGRDYTLTISSLEPEDFAVYYCQQW | | | | |
| | SRNPPTFGGGTKVEIKGGSEGKSSGSGSESKSTGGSQVQLVQSGA | | | | |
| | EVKKPGSSVKVSCKASGYTFTRSTMHWVKQAPGQGLEWIGYINPS | | | | |
| | SAYTNYNQKFQGRVTLTADKSTSTAYMELSSLRSEDTAVYYCASP | | | | |
| | QVHYDYAGFPYWGQGTLVTVSSEPKSSDKTHTCPPCPAPEAAGGP | | | | |
| | SVFLFPPKPKDTLMISRTPEVTCVVVSVSHEDPEVKFNWYVDGVE | | | | |
| | VHNAKTKPREEQYNSTYRVVSVLTVLHQDWLNGKEYKCKVSNKAL | | | | |
| | PAPIEKTISKAKGQPREPQVYTLPPSREEMTKNQVSLWCLVKGFY | | | | |
| | PSDIAVEWESNGQPENNYKTTPPVLDSDGSFFLYSKLTVDKSRWQ | | | | |
| | QGNVFSCSVMHEALHNHYTQKSLSLSPGK | | | | |
| PS3B1396 | (see above) | 1455 | NA | 1458 | CD3B2030- |
| | | | | | N106A- |
| | | | | | scFv-LH- |
| | | | | | CH2 |

TABLE 24
PSMA x CD3 Bispecific Antibodies: PSMA Arm Descriptions

| | | SEQ | | SEQ | PSMA Arm |
|---|---|---|---|---|---|
| | | ID | | ID | Descrip- |
| Name | HC2 | NO. | LC2 | NO. | tion |
| PS3B1353 | EVQLVESGGGLVQPGGSLRLSCAASGFTFSSYAMSWVRQAPGKGLE | 1242 | QSVLTQPPSV | 272 | PSMB947 |
| | WVSAISGGIGSTYYADSVKGRFTISRDNSKNTLWLQMNSLRAEDTA | | SAAPGQKVTI | | |
| | VYYCAKDGVGATPYYFDYWGQGTLVTVSSASTKGPSVFPLAPSSKS | | SCSGSSSNIG | | |
| | TSGGTAALGCLVKDYFPEPVTVSWNSGALTSGVHTFPAVLQSSGLY | | INYVSWYQQL | | |
| | SLSSVVTVPSSSLGTQTYICNVNHKPSNTKVDKKVEPKSCDKTHTC | | PGTAPKLLIY | | |
| | PPCPAPEAAGGPSVFLFPPKPKDTLMISRTPEVTCVVVSVSHEDPE | | DNNKRPSGIP | | |
| | VKFNWYVDGVEVHNAKTKPREEQYNSTYRVVSVLTVLHQDWLNGKE | | DRFSGSKSGT | | |
| | YKCKVSNKALPAPIEKTISKAKGQPREPQVYTLPPSREEMTKNQVS | | SATLGITGLQ | | |
| | LTCLVKGFYPSDIAVEWESNGQPENNYKTTPPVLDSDGSFFLYSRL | | TGDEADYYCG | | |
| | TVDKSRWQQGNVFSCSVMHEALHNHYTQKSLSLSPGK | | TWDSSLSAVV | | |
| | | | FGGGTKLTVL | | |
| | | | GQPKAAPSVT | | |
| | | | LFPPSSEELQ | | |
| | | | ANKATLVCLI | | |
| | | | SDFYPGAVTV | | |
| | | | AWKADSSPVK | | |
| | | | AGVETTTPSK | | |
| | | | QSNNKYAASS | | |
| | | | YLSLTPEQWK | | |
| | | | SHRSYSCQVT | | |
| | | | HEGSTVEKTV | | |
| | | | APTECS | | |
| PS3B1505 | EVQLVESGGGLVQPGGSLRLSCAASGFTFSSYAMSWVRQAPGKGLE | 1244 | (see | 272 | PSMB896- |
| | WVSAISGGIGSTYYADSVKGRFTISRDNSKNTLWLQMNSLRAEDTA | | above) | | G100A |
| | VYYCAKDAVGATPYYFDYWGQGTLVTVSSASTKGPSVFPLAPSSKS | | | | IgG1 |
| | TSGGTAALGCLVKDYFPEPVTVSWNSGALTSGVHTFPAVLQSSGLY | | | | DuoBody |
| | SLSSVVTVPSSSLGTQTYICNVNHKPSNTKVDKKVEPKSCDKTHTC | | | | |
| | PPCPAPEAAGGPSVFLFPPKPKDTLMISRTPEVTCVVVSVSHEDPE | | | | |
| | VKFNWYVDGVEVHNAKTKPREEQYNSTYRVVSVLTVLHQDWLNGKE | | | | |
| | YKCKVSNKALPAPIEKTISKAKGQPREPQVYTLPPSREEMTKNQVS | | | | |
| | LTCLVKGFYPSDIAVEWESNGQPENNYKTTPPVLDSDGSFFLYSRL | | | | |
| | TVDKSRWQQGNVFSCSVMHEALHNHYTQKSLSLSPGK | | | | |
| PS3B1508 | EVQLVESGGGLVQPGGSLRLSCAASGFTFSSYAMSWVRQAPGKGLE | 441 | (see | 272 | PSMB896- |
| | WVSAISGGIGSTYYADSVKGRFTISRDNSKNTLWLQMNSLRAEDTA | | above) | | G100A- |
| | VYYCAKDAVGATPYYFDYWGQGTLVTVSSASTKGPSVFPLAPSSKS | | | | Fab- |
| | TSGGTAALGCLVKDYFPEPVTVSWNSGALTSGVHTFPAVLQSSGLY | | | | RF: IgG1 |
| | SLSSVVTVPSSSLGTQTYICNVNHKPSNTKVDKKVEPKSCDKTHTC | | | | AAS |
| | PPCPAPEAAGGPSVFLFPPKPKDTLMISRTPEVTCVVVSVSHEDPE | | | | |
| | VKFNWYVDGVEVHNAKTKPREEQYNSTYRVVSVLTVLHQDWLNGKE | | | | |
| | YKCKVSNKALPAPIEKTISKAKGQPREPQVYTLPPSREEMTKNQVS | | | | |
| | LSCAVKGFYPSDIAVEWESNGQPENNYKTTPPVLDSDGSFFLVSKL | | | | |
| | TVDKSRWQQGNVFSCSVMHEALHNRFTQKSLSLSPGK | | | | |
| PS3B1917 | SYELMQPPSVSVSPGQTARITCSGDALPKQYAYWYQQKPGQAPVLV | 1248 | NA | 1249 | PSMA_ |
| | IYKDSERPSGIPVRFSGSSSGTTVTLTITGVQAEDEADYYCQSADS | | | | P72_ |
| | SGTYVFGTGTKVTVLGGSEGKSSGSGSESKSTGGSQVQLVESGGGV | | | | A10-HC- |
| | VQPGRSLRLSCAASGFTFSSYNMNWVRQAPGKGLEWVAIIYYDESN | | | | G54E |
| | KYYADSVKGRFTISRDISKNTLYLQMNSLRAEDTAVYYCARERGRD | | | | |
| | YYGMDVWGQGTTVTVSSEPKSSDKTHTCPPCPAPEAAGGPSVFLFP | | | | |
| | PKPKDTLMISRTPEVTCVVVSVSHEDPEVKFNWYVDGVEVHNAKTK | | | | |
| | PREEQYNSTYRVVSVLTVLHQDWLNGKEYKCKVSNKALPAPIEKTI | | | | |
| | SKAKGQPREPQVYVLPPSREEMTKNQVSLLCLVKGFYPSDIAVEWE | | | | |
| | SNGQPENNYLTWPPVLDSDGSFFLYSKLTVDKSRWQQGNVFSCSVM | | | | |
| | HEALHNHYTQKSLSLSPG | | | | |
| PS3B1918 | QSVLTQPASVSGSPGQSITISCTGTSSDVGGYNYVSWYQQHPGKAP | 1250 | NA | 1251 | PSMA_ |
| | KLMIYEVSNRPSGVSNRFSGSKSGNTASLTISGLQAEDEADYYCSS | | | | P72_ |
| | YTSSYTYVFGTGTKLTVLGGSEGKSSGSGSESKSTGGSEVQLVESG | | | | D01-HC- |
| | GDLVQPGGSLRLSCAASGFTFNNYNMNWVRQAPGKGLEWVSHISTS | | | | D95E |
| | SSNKYYADSVKGRFSISRDIAKNSMYLQMNSLRDEDTAVYYCAREG | | | | |
| | VGADYGDYYYYGMDVWGQGTTVTVSSEPKSSDKTHTCPPCPAPEAA | | | | |
| | GGPSVFLFPPKPKDTLMISRTPEVTCVVVSVSHEDPEVKFNWYVDG | | | | |
| | VEVHNAKTKPREEQYNSTYRVVSVLTVLHQDWLNGKEYKCKVSNKA | | | | |
| | LPAPIEKTISKAKGQPREPQVYVLPPSREEMTKNQVSLLCLVKGFY | | | | |
| | PSDIAVEWESNGQPENNYLTWPPVLDSDGSFFLYSKLTVDKSRWQQ | | | | |
| | GNVFSCSVMHEALHNHYTQKSLSLSPG | | | | |
| PS3B1919 | EIVLTQSPGTLSVSPGERATLSCRASQSVRSNLAWYQQKPGQAPRL | 1252 | NA | 1253 | PSMA_ |
| | LIYGASTRATGIPARFSGSGSGTEFTLTISSLQSEDFAVYYCHQYN | | | | P75_F |
| | DWPPYTFGQGTKLEIKGGSEGKSSGSGSESKSTGGSQVQLQESGGG | | | | 01 |
| | VVQPGRSLRLSCAASGFTFSTYGMHWVRQAPGKGLEWVAFISYDGS | | | | |
| | NKYYADSVKGRFTISRDNSKHTLYLQMNSLRAEDTAVYYCAGRDNL | | | | |
| | RFLEWFMDVWGQGTTVTVSSEPKSSDKTHTCPPCPAPEAAGGPSVF | | | | |
| | LFPPKPKDTLMISRTPEVTCVVVSVSHEDPEVKFNWYVDGVEVHNA | | | | |
| | KTKPREEQYNSTYRVVSVLTVLHQDWLNGKEYKCKVSNKALPAPIE | | | | |
| | KTISKAKGQPREPQVYVLPPSREEMTKNQVSLLCLVKGFYPSDIAV | | | | |
| | EWESNGQPENNYLTWPPVLDSDGSFFLYSKLTVDKSRWQQGNVFSC | | | | |
| | SVMHEALHNHYTQKSLSLSPG | | | | |
| PS3B1920 | SYELTQPPSVSVAPGQTARITCGGNNIGSKSVHWYQQKPGQAPVLV | 1254 | NA | 1255 | PSMA_ |
| | VYDDSDRPSGIPERFSGSNSGNTATLTISRVEAGDEADYYCQVWDS | | | | P72_F |
| | STDHVVFGGGTKLTVLGGSEGKSSGSGSESKSTGGSEVQLVESGGG | | | | 07 |
| | VVQPGRSLRLSCAASGFTFSSYGMNWVRQAPGKGLEWVAVTSYDGS | | | | |
| | NKYYADSVKGRFTISRDISKNTLYLQMSSLRAEDTAVYYCARDPYS | | | | |
| | SSWNGAFDIWGPGTMVTVSSEPKSSDKTHTCPPCPAPEAAGGPSVF | | | | |
| | LFPPKPKDTLMISRTPEVTCVVVSVSHEDPEVKFNWYVDGVEVHNA | | | | |
| | KTKPREEQYNSTYRVVSVLTVLHQDWLNGKEYKCKVSNKALPAPIE | | | | |
| | KTISKAKGQPREPQVYVLPPSREEMTKNQVSLLCLVKGFYPSDIAV | | | | |
| | EWESNGQPENNYLTWPPVLDSDGSFFLYSKLTVDKSRWQQGNVFSC | | | | |
| | SVMHEALHNHYTQKSLSLSPG | | | | |
| PS3B1921 | QSVLTQPPSASGTPGQGVTISCSGSSSNIGSNTVNWFQQLPGTAPK | 1256 | NA | 1257 | PSMA_ |
| | LLIYSDNQRPSGVPDRFSGSKSGTSASLAISGLQSEDEADYYCAAW | | | | P72_E |
| | DDSLNGYVFGTGTKVTVLGGSEGKSSGSGSESKSTGGSEVQLVESG | | | | 07 |
| | GGVVQPGRSLRLSCAASGFTFITYGMHWVRQAPGKGLEWVAVVSFD | | | | |
| | ESNKYYADSVKGRFTISRDNSKNTLYLQMNSLRAEDTAVYYCARAL | | | | |
| | RDGNNWDYFNGMDVWGQGTTVTVSSEPKSSDKTHTCPPCPAPEAAG | | | | |
| | GPSVFLFPPKPKDTLMISRTPEVTCVVVSVSHEDPEVKFNWYVDGV | | | | |
| | EVHNAKTKPREEQYNSTYRVVSVLTVLHQDWLNGKEYKCKVSNKAL | | | | |
| | PAPIEKTISKAKGQPREPQVYVLPPSREEMTKNQVSLLCLVKGFYP | | | | |
| | SDIAVEWESNGQPENNYLTWPPVLDSDGSFFLYSKLTVDKSRWQQG | | | | |
| | NVFSCSVMHEALHNHYTQKSLSLSPG | | | | |
| PS3B1922 | QSVLTQPASVSGSPGQSITISCTGTSSDVGGYNYVSWYQQHPGKAP | 1258 | NA | 1259 | PSMA_ |
| | KLMIYEVSNRPSGVSNRFSGSKSGNTASLTISGLQAEDEADYYCSS | | | | P72_ |
| | YTSSYTYVFGTGTKLTVLGGSEGKSSGSGSESKSTGGSEVQLVESG | | | | D01 |
| | GDLVQPGGSLRLSCAASGFTFNNYNMNWVRQAPGKGLEWVSHISTS | | | | |
| | SSNKYYADSVKGRFSISRDIAKNSMYLQMNSLRDEDTAVYYCARDG | | | | |
| | VGADYGDYYYYGMDVWGQGTTVTVSSEPKSSDKTHTCPPCPAPEAA | | | | |
| | GGPSVFLFPPKPKDTLMISRTPEVTCVVVSVSHEDPEVKFNWYVDG | | | | |
| | VEVHNAKTKPREEQYNSTYRVVSVLTVLHQDWLNGKEYKCKVSNKA | | | | |
| | LPAPIEKTISKAKGQPREPQVYVLPPSREEMTKNQVSLLCLVKGFY | | | | |
| | PSDIAVEWESNGQPENNYLTWPPVLDSDGSFFLYSKLTVDKSRWQQ | | | | |
| | GNVFSCSVMHEALHNHYTQKSLSLSPG | | | | |
| PS3B1923 | QSVLTQPPSVSVAPGQTARITCGGNNSGSKSVHWYQQKPGQAPVLV | 1260 | NA | 1261 | PSMA_ |
| | VYDDSDRPSGIPERFSGSNSGNTATLTISRVEAGDEADYYCQVWDS | | | | P72_C |
| | SSDHGVFGGGTKLTVLGGSEGKSSGSGSESKSTGGSQVQLVESGGG | | | | 01 |
| | EVQPGRSLRLSCAASGFSFSGYGMHWVRQAPGKGLEWVAVMSYDGS | | | | |
| | NRFYVDSVRGRFSISRDNSKNTLYLQMNSLRPEDTAVYYCARDTVW | | | | |
| | GSHPDAFDIWGQGTVVTVSSEPKSSDKTHTCPPCPAPEAAGGPSVF | | | | |
| | LFPPKPKDTLMISRTPEVTCVVVSVSHEDPEVKFNWYVDGVEVHNA | | | | |
| | KTKPREEQYNSTYRVVSVLTVLHQDWLNGKEYKCKVSNKALPAPIE | | | | |
| | KTISKAKGQPREPQVYVLPPSREEMTKNQVSLLCLVKGFYPSDIAV | | | | |
| | EWESNGQPENNYLTWPPVLDSDGSFFLYSKLTVDKSRWQQGNVFSC | | | | |
| | SVMHEALHNHYTQKSLSLSPG | | | | |
| PS3B1924 | SYELMQPPSVSVSPGQTARITCSGDALPKQYAYWYQQKPGQAPVLV | 1262 | NA | 1263 | PSMA_ |
| | IYKDSERPSGIPVRFSGSSSGTTVTLTITGVQAEDEADYYCQSADS | | | | P72_ |
| | SGTYVFGTGTKVTVLGGSEGKSSGSGSESKSTGGSQVQLVESGGGV | | | | A10 |
| | VQPGRSLRLSCAASGFTFSSYNMNWVRQAPGKGLEWVAIIYYDGSN | | | | |
| | KYYADSVKGRFTISRDISKNTLYLQMNSLRAEDTAVYYCARERGRD | | | | |
| | YYGMDVWGQGTTVTVSSEPKSSDKTHTCPPCPAPEAAGGPSVFLFP | | | | |
| | PKPKDTLMISRTPEVTCVVVSVSHEDPEVKFNWYVDGVEVHNAKTK | | | | |
| | PREEQYNSTYRVVSVLTVLHQDWLNGKEYKCKVSNKALPAPIEKTI | | | | |
| | SKAKGQPREPQVYVLPPSREEMTKNQVSLLCLVKGFYPSDIAVEWE | | | | |
| | SNGQPENNYLTWPPVLDSDGSFFLYSKLTVDKSRWQQGNVFSCSVM | | | | |
| | HEALHNHYTQKSLSLSPG | | | | |
| PS3B1925 | QSVLTQPPSASGTPGQRVTISCSGSSSNIGSNTVNWYQQLPGTAPK | 1264 | NA | 1265 | PSMA_ |
| | LLIYSSNQRPSGVPDRFSGSKSGTSASLAISGLQSEDEADYYCAAW | | | | P70_F |
| | DDSLNGVVFGGGTKLTVLGGSEGKSSGSGSESKSTGGSEVQLLESG | | | | 02 |
| | PGLVKPSETLSLTCTVSGGSIISYYWSWIRQPAGKGLEWIGRIYSS | | | | |
| | GSTNYNPSLKSRVTMSVDTSKNQFSLKLSSVTAADTAVYYCAKVGV | | | | |
| | WPGAFDIWGQGTMVTVSSEPKSSDKTHTCPPCPAPEAAGGPSVFLF | | | | |
| | PPKPKDTLMISRTPEVTCVVVSVSHEDPEVKFNWYVDGVEVHNAKT | | | | |
| | KPREEQYNSTYRVVSVLTVLHQDWLNGKEYKCKVSNKALPAPIEKT | | | | |
| | ISKAKGQPREPQVYVLPPSREEMTKNQVSLLCLVKGFYPSDIAVEW | | | | |
| | ESNGQPENNYLTWPPVLDSDGSFFLYSKLTVDKSRWQQGNVFSCSV | | | | |
| | MHEALHNHYTQKSLSLSPG | | | | |
| PS3B1926 | EVQLVESGGGVVQPGRSLRLSCAASGFSFSGYGMHWVRQAPGKGLE | 1266 | NA | 1267 | PSMA_ |
| | WVAVISYDGSNKYYADSVKGRFTISRDNSKNTLYLQMNSLRVEDTA | | | | P72_ |
| | VYYCARDRIWGSRGYYYGMDVWGQGTTVTVSSGGSEGKSSGSGSES | | | | G02 |
| | KSTGGSQSALTQPASVSGSPGQSITISCTGASSDVGGYNYVSWYQQ | | | | |
| | HPGKAPKLMIYEVSNRPSGVSNRFSGSKSGNTASLTISGLQAEDEA | | | | |
| | DYYCSSYTITSTLVFGGGTKLTVLEPKSSDKTHTCPPCPAPEAAGG | | | | |
| | PSVFLFPPKPKDTLMISRTPEVTCVVVSVSHEDPEVKFNWYVDGVE | | | | |
| | VHNAKTKPREEQYNSTYRVVSVLTVLHQDWLNGKEYKCKVSNKALP | | | | |
| | APIEKTISKAKGQPREPQVYVLPPSREEMTKNQVSLLCLVKGFYPS | | | | |
| | DIAVEWESNGQPENNYLTWPPVLDSDGSFFLYSKLTVDKSRWQQGN | | | | |
| | VFSCSVMHEALHNHYTQKSLSLSPG | | | | |
| PS3B1927 | QVQLVESGGGEVQPGRSLRLSCAASGFSFSGYGMHWVRQAPGKGLE | 1268 | NA | 1269 | PSMA_ |
| | WVAVMSYDGSNRFYVDSVRGRFSISRDNSKNTLYLQMNSLRPEDTA | | | | P72_C |
| | VYYCARDTVWGSHPDAFDIWGQGTVVTVSSGGSEGKSSGSGSESKS | | | | 01 |
| | TGGSQSVLTQPPSVSVAPGQTARITCGGNNSGSKSVHWYQQKPGQA | | | | |
| | PVLVVYDDSDRPSGIPERFSGSNSGNTATLTISRVEAGDEADYYCQ | | | | |
| | VWDSSSDHGVFGGGTKLTVLEPKSSDKTHTCPPCPAPEAAGGPSVF | | | | |
| | LFPPKPKDTLMISRTPEVTCVVVSVSHEDPEVKFNWYVDGVEVHNA | | | | |
| | KTKPREEQYNSTYRVVSVLTVLHQDWLNGKEYKCKVSNKALPAPIE | | | | |
| | KTISKAKGQPREPQVYVLPPSREEMTKNQVSLLCLVKGFYPSDIAV | | | | |
| | EWESNGQPENNYLTWPPVLDSDGSFFLYSKLTVDKSRWQQGNVFSC | | | | |
| | SVMHEALHNHYTQKSLSLSPG | | | | |
| PS3B1928 | QVQLQESGGDVVQPGRSLRLSCAASGFSFSGYGLHWVRQAPGRGLE | 1270 | NA | 1271 | PSMA_ |
| | WVTLISYDGSNKYYADSVKGRFTISRDNSKNTLYLQMNSLRAEDTA | | | | P72_ |
| | VYYCAKTTVSDPYYYGMDVWGQGTTVTVSSGGSEGKSSGSGSESKS | | | | A11 |
| | TGGSSYELTQPPSVSVAPGQTARITCGGNNIGSKSVHWYQQKPGQA | | | | |
| | PVLVVYDDSDRPSGIPERFSGTNSGNTATLTISRAEAGDEADYYCQ | | | | |
| | VWDSSSDHVVFGGGTKLTVLEPKSSDKTHTCPPCPAPEAAGGPSVF | | | | |
| | LFPPKPKDTLMISRTPEVTCVVVSVSHEDPEVKFNWYVDGVEVHNA | | | | |
| | KTKPREEQYNSTYRVVSVLTVLHQDWLNGKEYKCKVSNKALPAPIE | | | | |
| | KTISKAKGQPREPQVYVLPPSREEMTKNQVSLLCLVKGFYPSDIAV | | | | |
| | EWESNGQPENNYLTWPPVLDSDGSFFLYSKLTVDKSRWQQGNVFSC | | | | |
| | SVMHEALHNHYTQKSLSLSPG | | | | |
| PSMB1041 | (see above) | 1264 | NA | 1273 | PSMA_ |
| | | | | | P70_F |
| | | | | | 02 |
| PSMB1045 | (see above) | 1262 | NA | 1275 | PSMA_ |
| | | | | | P72_ |
| | | | | | A10 |
| PSMB1047 | (see above) | 1260 | NA | 1277 | PSMA_ |
| | | | | | P72_C |
| | | | | | 01 |
| PSMB1049 | (see above) | 1258 | NA | 1279 | PSMA_ |
| | | | | | P72_ |
| | | | | | D01 |
| PSMB1051 | (see above) | 1256 | NA | 1281 | PSMA_ |
| | | | | | P72_E |
| | | | | | 07 |
| PSMB1052 | (see above) | 1254 | NA | 1283 | PSMA_ |
| | | | | | P72_F |
| | | | | | 07 |
| PSMB1060 | (see above) | 1252 | NA | 1285 | PSMA_ |
| | | | | | P75_F |
| | | | | | 01 |
| PSMB1068 | (see above) | 1270 | NA | 1287 | PSMA_ |
| | | | | | P72_ |
| | | | | | A11 |
| PSMB1069 | (see above) | 1268 | NA | 1289 | PSMA_ |
| | | | | | P72_C |
| | | | | | 01 |
| PSMB1075 | (see above) | 1266 | NA | 1291 | PSMA_ |
| | | | | | P72_ |
| | | | | | G02 |
| PSMB2908 | (see above) | 1250 | NA | 1293 | PSMA_ |
| | | | | | P72_ |
| | | | | | D01-HC- |
| | | | | | D95E |
| PSMB2909 | (see above) | 1248 | NA | 1295 | PSMA_ |
| | | | | | P72_ |
| | | | | | A10-HC- |
| | | | | | G54E |
| PS3B1391 | EVQLVESGGGLVKPGGSLRLSCVASGFTFSFYSMNWVRQAPGKGLD | 407 | EIVMTQSPGT | 408 | PSMHB |
| | WVSSISSSGNYIYYADSVKGRFTISRDNAKNSLHLHMNSLKAEDTA | | LSLSPGERAT | | 49SC |
| | MYFCARSYSGSYDAFDFWGQGTMVTVSSASTKGPSVFPLAPSSKST | | LSCRASQSVS | | 1133_ |
| | SGGTAALGCLVKDYFPEPVTVSWNSGALTSGVHTFPAVLQSSGLYS | | SSFLAWYQQK | | 011A11 |
| | LSSVVTVPSSSLGTQTYICNVNHKPSNTKVDKKVEPKSCDKTHTCP | | PGQAPRLLIS | | _1 |
| | PCPAPEAAGGPSVFLFPPKPKDTLMISRTPEVTCVVVSVSHEDPEV | | GASSRATGIP | | |
| | KFNWYVDGVEVHNAKTKPREEQYNSTYRVVSVLTVLHQDWLNGKEY | | DRFSVSGSGT | | |
| | KCKVSNKALPAPIEKTISKAKGQPREPQVYTLPPSREEMTKNQVSL | | DFTLTISRLE | | |
| | SCAVKGFYPSDIAVEWESNGQPENNYKTTPPVLDSDGSFFLVSKLT | | PEDFAVYYCQ | | |
| | VDKSRWQQGNVFSCSVMHEALHNRFTQKSLSLSPGK | | QYGVSPWTFG | | |
| | | | QGTKVEIKRT | | |
| | | | VAAPSVFIFP | | |
| | | | PSDEQLKSGT | | |
| | | | ASVVCLLNNF | | |
| | | | YPREAKVQWK | | |
| | | | VDNALQSGNS | | |
| | | | QESVTEQDSK | | |
| | | | DSTYSLSSTL | | |
| | | | TLSKADYEKH | | |
| | | | KVYACEVTHQ | | |
| | | | GLSSPVTKSF | | |
| | | | NRGEC | | |
| PS3B1396 | (see above) | 441 | (see | 272 | PSMB |
| | | | above) | | 896- |
| | | | | | G100A- |
| | | | | | Fab |

TABLE 25
PSMA × CD3 Bispecific Antibodies: Clone Descriptions

| Name | Bispecific Description |
|---|---|
| PS3B917 | FAB-A: CD3B891 (CD3B376 − K477); |
| | FAB-B: PSMB889 |
| PS3B918 | FAB-A: CD3B891 (CD3B376 − K477); |
| | FAB-B: PSMB890 |
| PS3B913 | FAB-A: CD3B891 (CD3B376 − K477); |
| | FAB-B: PSMB891 |
| PS3B915 | FAB-A: CD3B891 (CD3B376 − K477); |
| | FAB-B: PSMB892 |
| PS3B914 | FAB-A: CD3B891 (CD3B376 − K477); |
| | FAB-B: PSMB893 |
| PS3B916 | FAB-A: CD3B891 (CD3B376 − K477); |
| | FAB-B: PSMB894 |
| PS3B919 | FAB-A: CD3B891 (CD3B376 − K477); |
| | FAB-B: PSMB895 |
| PS3B921 | FAB-A: CD3B891 (CD3B376 − K477); |
| | FAB-B: PSMB896 |
| PS3B920 | FAB-A: CD3B891 (CD3B376 − K477); |
| | FAB-B: PSMB897 |
| PS3B922 | FAB-A: CD3B891 (CD3B376 − K477); |
| | FAB-B: PSMB898 |
| PS3B912 | FAB-A: CD3B891 (CD3B376 − K477); |
| | FAB-B: PSMB899 |
| PS3B930 | FAB-A: CD3B2183 (CD3W245); |
| | FAB-B: PSMB889 |
| PS3B931 | FAB-A: CD3B2183 (CD3W245); |
| | FAB-B: PSMB890 |
| PS3B926 | FAB-A: CD3B2183 (CD3W245); |
| | FAB-B: PSMB891 |
| PS3B928 | FAB-A: CD3B2183 (CD3W245); |
| | FAB-B: PSMB892 |
| PS3B927 | FAB-A: CD3B2183 (CD3W245); |
| | FAB-B: PSMB893 |
| PS3B929 | FAB-A: CD3B2183 (CD3W245); |
| | FAB-B: PSMB894 |
| PS3B932 | FAB-A: CD3B2183 (CD3W245); |
| | FAB-B: PSMB895 |
| PS3B934 | FAB-A: CD3B2183 (CD3W245); |
| | FAB-B: PSMB896 |
| PS3B933 | FAB-A: CD3B2183 (CD3W245); |
| | FAB-B: PSMB897 |
| PS3B935 | FAB-A: CD3B2183 (CD3W245); |
| | FAB-B: PSMB898 |
| PS3B925 | FAB-A: CD3B2183 (CD3W245); |
| | FAB-B: PSMB899 |
| PS3B1352 | FAB-A: CD3B2197 (CD3B376 + K477); |
| | FAB-B: PSMB946 |
| PS3B1353 | FAB-A: CD3B2197 (CD3B376 + K477); |
| | FAB-B: PSMB947 |
| PS3B1354 | FAB-A: CD3B2197 (CD3B376 + K477); |
| | FAB-B: PSMB948 |
| PS3B1355 | FAB-A: CD3B2197 (CD3B376 + K477); |
| | FAB-B: PSMB949 |
| PS3B1356 | FAB-A: CD3B2200 (CD3B450 + K477); |
| | FAB-B: PSMB946 |
| PS3B1357 | FAB-A: CD3B2200 (CD3B450 + K477); |
| | FAB-B: PSMB947 |
| PS3B1358 | FAB-A: CD3B2200 (CD3B450 + K477); |
| | FAB-B: PSMB949 |
| PSMB937 | FAB-A: CD3B2186 (CD3B450 − K477); |
| | FAB-B: PSMB897 (PSMB948) |

TABLE 26
PSMA × CD3 Bispecific Antibodies: CD3 Arm Descriptions

| Name | HC1 | LC1 | CD3 Arm Description |
|---|---|---|---|
| PS3B917, PS3B918, | SEQ ID | SEQ ID | with CD3B376 arm (CD3B891 without K477 or CD3B2197 with K477 in HC1) |
| PS3B913, PS3B915, | NO: 883 | NO: 850 | HC1 Construct ID: PBD000100300 |
| PS3B914, PS3B916, | | | LC1 Contruct ID: PBD000044707 |
| PS3B919, PS3B921, | | | |
| PS3B920, PS3B922, | | | |
| PS3B912 | | | |
| PS3B930, PS3B931, | SEQ ID | SEQ ID | with CD3W245 arm (CD3B2183 without K477) |
| PS3B926, PS3B928, | NO:1167 | NO: 986 | HC1 Construct ID: PBD000100302 |
| PS3B927, PS3B929, | | | LC1 Contruct ID: PBD000084982 |
| PS3B932, PS3B934, | | | |
| PS3B933, PS3B935, | | | |
| PS3B925 | | | |
| PS3B1352, PS3B1354, | SEQ ID | SEQ ID | with CD3B376 arm (CD3B891 without K477 or CD3B2197 with K477 in HC1) |
| PS3B1355, PS3B1356, | NO: 849 | NO: 850 | HC1 Construct ID: PBD000108469 |
| PS3B1357, PS3B1358 | | | LC1 Contruct ID: PBD000108469 |
| PSMB937 | SEQ ID | SEQ ID | with CD3B450 arm (CD3B2186 without K477 or CD3B2200 with K477 in HC1) |
| | NO: 951 | NO: 918 | HC1 Construct ID: PBD000100305 |
| | | | LC1 Contruct ID: PBD000045576 |

TABLE 27
PSMA x CD3 Bispecific Antibodies: PSMA Arm Descriptions

| | | SEQ | | SEQ | PSMA Arm |
|---|---|---|---|---|---|
| | | ID | | ID | Descrip- |
| Name | HC2 | NO. | LC2 | NO. | tion |
| PS3B | EVQLVESGGGLVKPGGSLRLSCAASGFTFSRYNMNWVRQAPG | 33 | QSVLTQPP | 34 | PSMB889 |
| 917 | KGLEWVSSINSNSRYIYYADSVKGRFTISRDSAKNSLYLQMN | | SVSGAPGQ | | HC2 |
| | SLRAEDTAVYYCAKTMGDYYYYYGMDVWGQGTTVTVSSASTK | | RVTISCTG | | Construct |
| | GPSVFPLAPSSKSTSGGTAALGCLVKDYFPEPVTVSWNSGAL | | SSFNLGAG | | ID: |
| | TSGVHTFPAVLQSSGLYSLSSVVTVPSSSLGTQTYICNVNHK | | YDVHWYQQ | | PBD000101312 |
| | PSNTKVDKKVEPKSCDKTHTCPPCPAPEAAGGPSVFLFPPKP | | VPGTVPKL | | |
| | KDTLMISRTPEVTCVVVSVSHEDPEVKFNWYVDGVEVHNAKT | | LIYDNSNR | | |
| | KPREEQYNSTYRVVSVLTVLHQDWLNGKEYKCKVSNKALPAP | | PSGVPDRF | | |
| | IEKTISKAKGQPREPQVYTLPPSREEMTKNQVSLTCLVKGFY | | SGSKSGTS | | |
| | PSDIAVEWESNGQPENNYKTTPPVLDSDGSFFLYSRLTVDKS | | ASLAITGL | | |
| | RWQQGNVFSCSVMHEALHNHYTQKSLSLSPG | | QAEDETVY | | |
| | | | YCQSYDSS | | |
| | | | LSGVVFGG | | |
| | | | GTKLTVLG | | |
| | | | QPKAAPSV | | |
| | | | TLFPPSSE | | |
| | | | ELQANKAT | | |
| | | | LVCLISDF | | |
| | | | YPGAVTVA | | |
| | | | WKADSSPV | | |
| | | | KAGVETTT | | |
| | | | PSKQSNNK | | |
| | | | YAASSYLS | | |
| | | | LTPEQWKS | | |
| | | | HRSYSCQV | | |
| | | | THEGSTVE | | |
| | | | KTVAPTEC | | |
| | | | S | | |
| PS3B | (see above) | 33 | SSELTQPP | 68 | PSMB890 |
| 918 | | | SVSGAPGQ | | HC2 |
| | | | RVTISCAG | | |
| | | | SLSNIGAG | | |
| | | | YDVHWYQQ | | |
| | | | LPGTAPKL | | |
| | | | LIYGNINR | | |
| | | | LSGVPERF | | |
| | | | SGSKSGTS | | |
| | | | ASLAITGL | | |
| | | | QAEDGADY | | |
| | | | YCQSYDSS | | Construct |
| | | | LSSYVFGT | | ID: |
| | | | GTKVTVLG | | PBD000101312 |
| | | | QPKAAPSV | | |
| | | | TLFPPSSE | | |
| | | | ELQANKAT | | |
| | | | LVCLISDF | | |
| | | | YPGAVTVA | | |
| | | | WKADSSPV | | |
| | | | KAGVETTT | | |
| | | | PSKQSNNK | | |
| | | | YAASSYLS | | |
| | | | LTPEQWKS | | |
| | | | HRSYSCQV | | |
| | | | THEGSTVE | | |
| | | | KTVAPTEC | | |
| | | | S | | |
| PS3B | EVQLVESGGGVVQPGRSLRLSCAASGFTFITYGMHWVRQAPG | 101 | QSVLTQPP | 102 | PSMB891 |
| 913 | KGLEWVAVVSFDESNKYYADSVKGRFTISRDNSKNTLYLQMN | | SASGTPGQ | | HC2 |
| | SLRAEDTAVYYCARALRDGNNWDYFNGMDVWGQGTTVTVSSA | | GVTISCSG | | Construct |
| | STKGPSVFPLAPSSKSTSGGTAALGCLVKDYFPEPVTVSWNS | | SSSNIGSN | | ID: |
| | GALTSGVHTFPAVLQSSGLYSLSSVVTVPSSSLGTQTYICNV | | TVNWFQQL | | PBD000101316 |
| | NHKPSNTKVDKKVEPKSCDKTHTCPPCPAPEAAGGPSVFLFP | | PGTAPKLL | | |
| | PKPKDTLMISRTPEVTCVVVSVSHEDPEVKFNWYVDGVEVHN | | IYSDNQRP | | |
| | AKTKPREEQYNSTYRVVSVLTVLHQDWLNGKEYKCKVSNKAL | | SGVPDRFS | | |
| | PAPIEKTISKAKGQPREPQVYTLPPSREEMTKNQVSLTCLVK | | GSKSGTSA | | |
| | GFYPSDIAVEWESNGQPENNYKTTPPVLDSDGSFFLYSRLTV | | SLAISGLQ | | |
| | DKSRWQQGNVFSCSVMHEALHNHYTQKSLSLSPG | | SEDEADYY | | |
| | | | CAAWDDSL | | |
| | | | NGYVFGTG | | |
| | | | TKVTVLGQ | | |
| | | | PKAAPSVT | | |
| | | | LFPPSSEE | | |
| | | | LQANKATL | | |
| | | | VCLISDFY | | |
| | | | PGAVTVAW | | |
| | | | KADSSPVK | | |
| | | | AGVETTTP | | |
| | | | SKQSNNKY | | |
| | | | AASSYLSL | | |
| | | | TPEQWKSH | | |
| | | | RSYSCQVT | | |
| | | | HEGSTVEK | | |
| | | | TVAPTECS | | |
| PS3B | (see above) | 101 | QSVLTQPP | 136 | PSMB892 |
| 915 | | | SVSGAPGQ | | HC2 |
| | | | RVTISCTG | | Construct |
| | | | SSSNIGAD | | ID: |
| | | | YDVHWYQH | | PBD000101316 |
| | | | LPGTAPKL | | |
| | | | LIYGNSNR | | |
| | | | PSGVPDRF | | |
| | | | SGSKSGTS | | |
| | | | ASLAITGL | | |
| | | | QAEDETDY | | |
| | | | YCQSYDSS | | |
| | | | LSGWVFGG | | |
| | | | GTKLTVLG | | |
| | | | QPKAAPSV | | |
| | | | TLFPPSSE | | |
| | | | ELQANKAT | | |
| | | | LVCLISDF | | |
| | | | YPGAVTVA | | |
| | | | WKADSSPV | | |
| | | | KAGVETTT | | |
| | | | PSKQSNNK | | |
| | | | YAASSYLS | | |
| | | | LTPEQWKS | | |
| | | | HRSYSCQV | | |
| | | | THEGSTVE | | |
| | | | KTVAPTEC | | |
| | | | S | | |
| PS3B | QVQLVESGGGVVQPGRSLRLSCVASGFTFSSYGIHWVRQAPG | 169 | (see | 102 | PSMB893 |
| 914 | KGLEWVAVIWYDGSNKYYADSVKGRFTISRDNSKNTLYLQMN | | above) | | HC2 |
| | SLRAEDTAVYYSVRGVGPTSYYYNYGMDVWGQGTTVTVSSAS | | | | Construct |
| | TKGPSVFPLAPSSKSTSGGTAALGCLVKDYFPEPVTVSWNSG | | | | ID: |
| | ALTSGVHTFPAVLQSSGLYSLSSVVTVPSSSLGTQTYICNVN | | | | PBD000101318 |
| | HKPSNTKVDKKVEPKSCDKTHTCPPCPAPEAAGGPSVFLFPP | | | | |
| | KPKDTLMISRTPEVTCVVVSVSHEDPEVKFNWYVDGVEVHNA | | | | |
| | KTKPREEQYNSTYRVVSVLTVLHQDWLNGKEYKCKVSNKALP | | | | |
| | APIEKTISKAKGQPREPQVYTLPPSREEMTKNQVSLTCLVKG | | | | |
| | FYPSDIAVEWESNGQPENNYKTTPPVLDSDGSFFLYSRLTVD | | | | |
| | KSRWQQGNVFSCSVMHEALHNHYTQKSLSLSPG | | | | |
| PS3B | (see above) | 169 | (see | 136 | PSMB894 |
| 916 | | | above) | | HC2 |
| | | | | | Construct |
| | | | | | ID: |
| | | | | | PBD000101318 |
| PS3B | EVQLVESGGGLVQPGGSLRLSCAASGFTFSSYAMSWVRQAPG | 237 | QSVLTQPP | 238 | PSMB895 |
| 919 | KGLEWVSAISGGIGSTYYADSVKGRFTISRDNSKNTLWLQMN | | SVSAAPGQ | | HC2 |
| | SLRAEDTAVYYCAKDGVGATPYYFDYWGQGTLVTVSSASTKG | | KVTISCSG | | Construct |
| | PSVFPLAPSSKSTSGGTAALGCLVKDYFPEPVTVSWNSGALT | | SSSNIGNN | | ID: |
| | SGVHTFPAVLQSSGLYSLSSVVTVPSSSLGTQTYICNVNHKP | | YVSWYQQL | | PBD000101320 |
| | SNTKVDKKVEPKSCDKTHTCPPCPAPEAAGGPSVFLFPPKPK | | PGTAPKLL | | |
| | DTLMISRTPEVTCVVVSVSHEDPEVKFNWYVDGVEVHNAKTK | | IYDNNKRP | | |
| | PREEQYNSTYRVVSVLTVLHQDWLNGKEYKCKVSNKALPAPI | | SGIPDRFS | | |
| | EKTISKAKGQPREPQVYTLPPSREEMTKNQVSLTCLVKGFYP | | GSKSGTSA | | |
| | SDIAVEWESNGQPENNYKTTPPVLDSDGSFFLYSRLTVDKSR | | TLGITGLQ | | |
| | WQQGNVFSCSVMHEALHNHYTQKSLSLSPG | | TGDEADYY | | |
| | | | CGTWDSSL | | |
| | | | SAYVFGTG | | |
| | | | TKVTVLGQ | | |
| | | | PKAAPSVT | | |
| | | | LFPPSSEE | | |
| | | | LQANKATL | | |
| | | | VCLISDFY | | |
| | | | PGAVTVAW | | |
| | | | KADSSPVK | | |
| | | | AGVETTTP | | |
| | | | SKQSNNKY | | |
| | | | AASSYLSL | | |
| | | | TPEQWKSH | | |
| | | | RSYSCQVT | | |
| | | | HEGSTVEK | | |
| | | | TVAPTECS | | |
| PS3B | (see above) | 237 | QSVLTQPP | 272 | PSMB896 |
| 921 | | | SVSAAPGQ | | HC2 |
| | | | KVTISCSG | | Construct |
| | | | SSSNIGIN | | ID: |
| | | | YVSWYQQL | | PBD000101320 |
| | | | PGTAPKLL | | |
| | | | IYDNNKRP | | |
| | | | SGIPDRFS | | |
| | | | GSKSGTSA | | |
| | | | TLGITGLQ | | |
| | | | TGDEADYY | | |
| | | | CGTWDSSL | | |
| | | | SAVVFGGG | | |
| | | | TKLTVLGQ | | |
| | | | PKAAPSVT | | |
| | | | LFPPSSEE | | |
| | | | LQANKATL | | |
| | | | VCLISDFY | | |
| | | | PGAVTVAW | | |
| | | | KADSSPVK | | |
| | | | AGVETTTP | | |
| | | | SKQSNNKY | | |
| | | | AASSYLSL | | |
| | | | TPEQWKSH | | |
| | | | RSYSCQVT | | |
| | | | HEGSTVEK | | |
| | | | TVAPTECS | | |
| PS3B | EVQLVESGGGLVQPGGSLRLSCAASGFTFSSYAMSWVRQAPG | 305 | (see | 238 | PSMB897 |
| 920 | KGLEWVSAISGGSGSTYYADSVKGRFTISRDNSKNTLYLQMN | | above) | | HC2 |
| | SLRAEDSAVYYCAKDGVGATPYYFDYWGQGTLVTVSSASTKG | | | | Construct |
| | PSVFPLAPSSKSTSGGTAALGCLVKDYFPEPVTVSWNSGALT | | | | ID: |
| | SGVHTFPAVLQSSGLYSLSSVVTVPSSSLGTQTYICNVNHKP | | | | PBD000101322 |
| | SNTKVDKKVEPKSCDKTHTCPPCPAPEAAGGPSVFLFPPKPK | | | | |
| | DTLMISRTPEVTCVVVSVSHEDPEVKFNWYVDGVEVHNAKTK | | | | |
| | PREEQYNSTYRVVSVLTVLHQDWLNGKEYKCKVSNKALPAPI | | | | |
| | EKTISKAKGQPREPQVYTLPPSREEMTKNQVSLTCLVKGFYP | | | | |
| | SDIAVEWESNGQPENNYKTTPPVLDSDGSFFLYSRLTVDKSR | | | | |
| | WQQGNVFSCSVMHEALHNHYTQKSLSLSPG | | | | |
| PS3B | (see above) | 305 | (see | 272 | PSMB898 |
| 922 | | | above) | | HC2 |
| | | | | | Construct |
| | | | | | ID: |
| | | | | | PBD000101322 |
| PS3B | EVQLVESGGGLVQPGGSLRLSCTASGFIFSSYAMSWVRQAPG | 373 | QSVLTQPP | 374 | PSMB899 |
| 912 | KGLEWVSAISGGYGAPYYADTVKGRFTISRDNSKNTLYLQMN | | SVSAAPGQ | | HC2 |
| | SLRAEDTAVYYCAKDGVGATPYYFDDWGQGILVTVSSASTKG | | KVTISCSG | | Construct |
| | PSVFPLAPSSKSTSGGTAALGCLVKDYFPEPVTVSWNSGALT | | SSSNIGNN | | ID: |
| | SGVHTFPAVLQSSGLYSLSSVVTVPSSSLGTQTYICNVNHKP | | YVSWYQQL | | PBD000101324 |
| | SNTKVDKKVEPKSCDKTHTCPPCPAPEAAGGPSVFLFPPKPK | | PGTAPKLL | | |
| | DTLMISRTPEVTCVVVSVSHEDPEVKFNWYVDGVEVHNAKTK | | IFDNNKRP | | |
| | PREEQYNSTYRVVSVLTVLHQDWLNGKEYKCKVSNKALPAPI | | SGIPDRFS | | |
| | EKTISKAKGQPREPQVYTLPPSREEMTKNQVSLTCLVKGFYP | | GSKSGTSA | | |
| | SDIAVEWESNGQPENNYKTTPPVLDSDGSFFLYSRLTVDKSR | | TLGITGLQ | | |
| | WQQGNVFSCSVMHEALHNHYTQKSLSLSPG | | TGDEADYY | | |
| | | | CGTWDSSL | | |
| | | | SAYVFGTG | | |
| | | | TKVTVLGQ | | |
| | | | PKAAPSVT | | |
| | | | LFPPSSEE | | |
| | | | LQANKATL | | |
| | | | VCLISDFY | | |
| | | | PGAVTVAW | | |
| | | | KADSSPVK | | |
| | | | AGVETTTP | | |
| | | | SKQSNNKY | | |
| | | | AASSYLSL | | |
| | | | TPEQWKSH | | |
| | | | RSYSCQVT | | |
| | | | HEGSTVEK | | |
| | | | TVAPTECS | | |
| PS3B | (see above) | 33 | (see | 34 | PSMB889 |
| 930 | | | above) | | HC2 |
| | | | | | Construct |
| | | | | | ID: |
| | | | | | PBD000101312 |
| PS3B | (see above) | 33 | (see | 68 | PSMB890 |
| 931 | | | above) | | HC2 |
| | | | | | Construct |
| | | | | | ID: |
| | | | | | PBD000101312 |
| PS3B | (see above) | 101 | (see | 102 | PSMB891 |
| 926 | | | above) | | HC2 |
| | | | | | Construct |
| | | | | | ID: |
| | | | | | PBD000101316 |
| PS3B | (see above) | 101 | (see | 136 | PSMB892 |
| 928 | | | above) | | HC2 |
| | | | | | Construct |
| | | | | | ID: |
| | | | | | PBD000101316 |
| PS3B | (see above) | 169 | (see | 102 | PSMB893 |
| 927 | | | above) | | HC2 |
| | | | | | Construct |
| | | | | | ID: |
| | | | | | PBD000101318 |
| PS3B | (see above) | 169 | (see | 136 | PSMB894 |
| 929 | | | above) | | HC2 |
| | | | | | Construct |
| | | | | | ID: |
| | | | | | PBD000101318 |
| PS3B | (see above) | 237 | (see | 238 | PSMB895 |
| 932 | | | above) | | HC2 |
| | | | | | Construct |
| | | | | | ID: |
| | | | | | PBD000101320 |
| PS3B | (see above) | 237 | (see | 272 | PSMB896 |
| 934 | | | above) | | HC2 |
| | | | | | Construct |
| | | | | | ID: |
| | | | | | PBD000101320 |
| PS3B | (see above) | 305 | (see | 238 | PSMB897 |
| 933 | | | above) | | HC2 |
| | | | | | Construct |
| | | | | | ID: |
| | | | | | PBD000101322 |
| PS3B | (see above) | 305 | (see | 272 | PSMB898 |
| 935 | | | above) | | HC2 |
| | | | | | Construct |
| | | | | | ID: |
| | | | | | PBD000101322 |
| PS3B | (see above) | 373 | (see | 374 | PSMB899 |
| 925 | | | above) | | HC2 |
| | | | | | Construct |
| | | | | | ID: |
| | | | | | PBD000101324 |
| PS3B | EVQLVESGGGLVQPGGSLRLSCAASGFTFSSYAMSWVRQAPG | 1242 | (see | 238 | PSMB946 |
| 1352 | KGLEWVSAISGGIGSTYYADSVKGRFTISRDNSKNTLWLQMN | | above) | | HC2 |
| | SLRAEDTAVYYCAKDGVGATPYYFDYWGQGTLVTVSSASTKG | | | | Construct |
| | PSVFPLAPSSKSTSGGTAALGCLVKDYFPEPVTVSWNSGALT | | | | ID: |
| | SGVHTFPAVLQSSGLYSLSSVVTVPSSSLGTQTYICNVNHKP | | | | PBD000108502 |
| | SNTKVDKKVEPKSCDKTHTCPPCPAPEAAGGPSVFLFPPKPK | | | | |
| | DTLMISRTPEVTCVVVSVSHEDPEVKFNWYVDGVEVHNAKTK | | | | |
| | PREEQYNSTYRVVSVLTVLHQDWLNGKEYKCKVSNKALPAPI | | | | |
| | EKTISKAKGQPREPQVYTLPPSREEMTKNQVSLTCLVKGFYP | | | | |
| | SDIAVEWESNGQPENNYKTTPPVLDSDGSFFLYSRLTVDKSR | | | | |
| | WQQGNVFSCSVMHEALHNHYTQKSLSLSPGK | | | | |
| PS3B | (see above) | 1242 | (see | 272 | PSMB947 |
| 1353 | | | above) | | HC2 |
| | | | | | Construct |
| | | | | | ID: |
| | | | | | PBD000108503 |
| PS3B | EVQLVESGGGLVQPGGSLRLSCAASGFTFSSYAMSWVRQAPG | 1404 | (see | 238 | PSMB848 |
| 1354 | KGLEWVSAISGGSGSTYYADSVKGRFTISRDNSKNTLYLQMN | | above) | | HC2 |
| | SLRAEDSAVYYCAKDGVGATPYYFDYWGQGTLVTVSSASTKG | | | | Construct |
| | PSVFPLAPSSKSTSGGTAALGCLVKDYFPEPVTVSWNSGALT | | | | ID: |
| | SGVHTFPAVLQSSGLYSLSSVVTVPSSSLGTQTYICNVNHKP | | | | PBD000108504 |
| | SNTKVDKKVEPKSCDKTHTCPPCPAPEAAGGPSVFLFPPKPK | | | | |
| | DTLMISRTPEVTCVVVSVSHEDPEVKFNWYVDGVEVHNAKTK | | | | |
| | PREEQYNSTYRVVSVLTVLHQDWLNGKEYKCKVSNKALPAPI | | | | |
| | EKTISKAKGQPREPQVYTLPPSREEMTKNQVSLTCLVKGFYP | | | | |
| | SDIAVEWESNGQPENNYKTTPPVLDSDGSFFLYSRLTVDKSR | | | | |
| | WQQGNVFSCSVMHEALHNHYTQKSLSLSPGK | | | | |
| PS3B | (see above) | 1404 | (see | 272 | PSMB849 |
| 1355 | | | above) | | HC2 |
| | | | | | Construct |
| | | | | | ID: |
| | | | | | PBD000108505 |
| PS3B | (see above) | 1242 | (see | 238 | PSMB946 |
| 1356 | | | above) | | HC2 |
| | | | | | Construct |
| | | | | | ID: |
| | | | | | PBD000108502 |
| PS3B | (see above) | 1242 | (see | 272 | PSMB847 |
| 1357 | | | above) | | HC2 |
| | | | | | Construct |
| | | | | | ID: |
| | | | | | PBD000108503 |
| PS3B | (see above) | 1404 | (see | 272 | PSMB949 |
| 1358 | | | above) | | HC2 |
| | | | | | Construct |
| | | | | | ID: |
| | | | | | PBD000108505 |
| PSMB | (see above) | 305 | (see | 238 | PSMB948 |
| 937 | | | above) | | HC2 |
| | | | | | Construct |
| | | | | | ID: |
| | | | | | PBD000101322 |

### Example 5.2: Analytical Characterization of Bispecific Anti-PSMAxCD3 Antibodies

The protein concentration for each purified bispecific Ab was determined by measuring the absorbance at 280 nm on a NANODROP1000 spectrophotometer or Trinean DROPSENSE96 multichannel spectrophotometer and calculated using the extinction coefficient based on the amino acid sequence. SE HPLC of the purified antibodies was performed by running samples on a TOSOH TSKgel BioAssist G3SWxl column, in 0.2 M Na Phosphate pH 6.8 at 1 mL/min on a Waters Alliance HPLC for 20 min. The column effluent was monitored by absorbance at 280 nm. Anti-PSMA-CD3 bispecific antibodies were further analyzed by Intact Mass Analysis to determine appropriate formation of heterodimers.

### Example 6: Epitope Mapping of Anti-PSMAxCD3 Antibodies

### Example 6.1: HDX-MS Epitope Mapping

The epitope of two anti-PSMA/CD3 bispecific antibodies PS3B1352 and PS3B1353 were determined by hydrogen-deuterium exchange mass spectrometry (HDX-MS). Human PSMA antigen was used for epitope mapping experiment.

On-Exchange Experiment for HDX-MS. Briefly, purified PSMA antigen was incubated with and without PSMA/CD3 bispecific antibody PS3B1352 or PS3B1353 in deuterium oxide labeled buffer. On-exchange reaction was initiated by mixing 10 μL, of 6.0 μM human PSMA with or without 7.3 μM antibody and 30 μL of H₂O or a deuterated buffer (20 mM MES, pH 6.4, 150 mM NaCl in 95% D₂O or 20 mM Tris, pH 8.4, 150 mM NaCl in 95% D20). The reaction mixture was incubated for 15, 50, 150, 500, or 1,500 s at 23° C. and quenched at the different time points described by the addition of 8 M urea, 1 M TCEP, pH 3.0. The quenched solutions were analyzed immediately.

General Procedure for HDX-MS Data Acquisition. HDX-MS sample preparation was performed using an automated HDx system (LEAP Technologies, Morrisville, NC). The samples were loaded through aprotease type XIII (protease from Aspergillus saitoi, type XIII)/pepsin column (NovaBioAssays Inc) at 600 μL/min in 1% acetonitrile, 0.1% formic acid. Peptide fragments were loaded on the trap column ACQUITY UPLC BEH C18 VanGuard Pre-column (Waters, Milford, MA), with a linear gradient of 8% to 28% acetonitrile in 0.1% formic acid at 100 μL/min over 20 min and analyzed by mass spectrometry. The analytical column used was a Accucore C18 (Thermo Fisher Scientific, Waltham, MA

MS Data Acquisition. Mass spectrometric analyses were carried out using an LTQ™ Orbitrap Fusion Lumos mass spectrometer (Thermo Fisher Scientific) with the capillary temperature at 275° C., resolution 150,000, and mass range (m/z) 300-2,000.

HDX-MS Data Extraction. BioPharma Finder 2.0 (Thermo Fisher Scientific) was used for the peptide identification of non-deuterated samples prior to the HDX experiments. HDExaminer version 2.4 (Sierra Analytics, Modesto, CA) was used to extract centroid values from the MS raw data files for the HDX experiments.

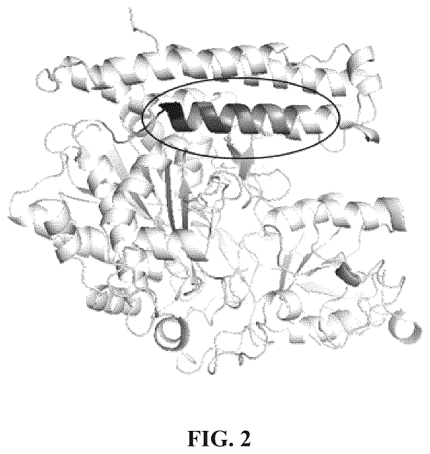

Incubation of PS3B1352 and PS3B1353 antibodies with soluble PSMA protein resulted in identical patterns of hydrogen exchange and overall protection (FIG. 1). Residues 597-598 (CR) were identified as epitopes because they were significantly protected upon binding (average differences in deuteration levels>10%). Residues 599 (D), 602-603 (VV) and 605 (R) were also identified as epitopes because they were marginally protected upon binding (average differences in deuteration levels 5%-10%). They may show bigger protections under longer time points. The binding epitopes could be larger than the four segments described herein. Residues located around the four segments describes above, such as 593-594 (LP), 595 (F), 596 (D), 600 (Y), 601 (A), 604 (L), 606-607 (KY), 607-609 (YAD), and 610-612, (MY), did not exchange at all in the time window employed, but could be protected if longer time points were to be monitored. The protected segments were mapped onto the three-dimensional structure of PSMA to visualize the binding epitopes (FIG. 2)

### Example 7: Characterization of Bispecific Anti-PSMAxCD3 Antibodies

### Example 7.1: Binding Affinity of Bispecific Anti-PSMAxCD3 Antibodies to Human PSMA

The binding affinity of anti-PSMA to the recombinant human, cynomolgus or mouse PSMA was determined by surface plasmon resonance (SPR) using a Biacore 8K instrument\. The bispecific antibodies were captured on a goat anti-Fc antibody-modified C1 chip and titrated with 3-fold serial dilutions of PSMA antigen spanning concentrations from 1 nM to 100 nM or 11.1 nM to 100 nM. The association and dissociation were monitored for 3 and 15 minutes, respectively, using a flow rate of 50 μL/min. Raw binding data was referenced by subtracting the analyte binding signals from blanks and analyzed using a 1:1 Langmuir binding model using the Biacore Insight evaluation software to obtain the kinetics, which were used to calculate the binding affinity. Kd data are summarized in Table 28. The anti-PSMA were captured using an anti-human Fc antibody and the antigens were injected in solution.

TABLE 28
Affinities (KD) for the interaction of anti-PSMA × CD3 bispecific
antibodies or anti-PSMA antibodies with human PSMA as
obtained by the BIACORE (SPR) method.

| Anti-PSMAxCD3 | | Anti-PSMA | | | |
|---|---|---|---|---|---|
| Name | KD (M) | Name | ka (1/Ms) | kd (1/s) | KD (M) |
| PS3B917 | ≤2.33E−11 | PSMB1045 | 2.09E+05 | 6.98E−05 | 3.33E−10 |
| PS3B918 | poor fit | PSMB1049 | 1.07E+05 | 7.02E−05 | 6.57E−10 |
| PS3B913 | ≤2.40E−10 | PSMB1051 | 7.71E+04 | 7.75E−05 | 1.01E−09 |
| PS3B915 | 1.89E−09 | PSMB1041 | 2.07E+05 | 4.35E−04 | 2.11E−09 |
| PS3B914 | Low/ | PSMB1068 | 1.51E+05 | 1.68E−04 | 1.11E−09 |
| | No binding | | | | |
| PS3B916 | 1.75E−09 | PSMB1052 | 1.96E+05 | 3.15E−04 | 1.61E−09 |
| PS3B919 | 8.23E−10 | PSMB1069 | 1.39E+05 | 1.97E−04 | 1.42E−09 |
| PS3B921 | ≤8.64E−11 | PSMB1047 | 6.94E+05 | 1.75E−03 | 2.52E−09 |
| PS3B920 | 1.35E−09 | PSMB1075 | 5.26E+05 | 4.92E−04 | 9.36E−10 |
| PS3B922 | 4.10E−10 | PSMB1060 | 1.17E+05 | 1.19E−04 | 1.02E−09 |
| PS3B912 | 1.74E−10 | | | | |
| PS3B930 | <2.26E−11 | | | | |
| PS3B931 | poor fit | | | | |
| PS3B926 | ≤2.53E−10 | | | | |
| PS3B928 | 2.37E−09 | | | | |
| PS3B927 | 1.11E−09 | | | | |
| PS3B929 | 1.83E−09 | | | | |
| PS3B932 | 7.87E−10 | | | | |
| PS3B934 | ≤8.73E−11 | | | | |
| PS3B933 | 1.44E−09 | | | | |
| PS3B935 | 4.54E−10 | | | | |
| PS3B925 | 1.69E−10 | | | | |

In addition, the binding affinity of selected anti-PSMA-CD3 bispecific antibodies to recombinant human PSMA was compared to the binding affinity to cynomolgus PSMA by SPR. The antibodies were captured on a goat anti-Fc antibody-modified C1 chip and titrated with 3-fold serial dilutions of PSMA antigen spanning concentrations of 100 nM to 3.7 nM (human PSMA) or 100 nM to 3.7 nM, or 22.2-600 nM for cyno PSMA. The association and dissociation were monitored for 3 and 60 minutes, respectively, using a flow rate of 50 μL/min. Raw binding data was referenced by subtracting the analyte binding signals from blanks and analyzed using a 1:1 Langmuir binding model by the Biacore Insight evaluation software to obtain the kinetics, which were used to calculate the binding affinity. The kinetic parameter of binding of selected antibodies are shown in Table 29. The anti-PSMA were captured using an anti-human Fc antibody and the antigens were injected in solution.

TABLE 29
Affinities (K_{D})* for the interaction of anti-PSMA antibodies with Human,
cyno or Mouse PSMA as obtained by the Biacore (SPR) method.

| | | Avg. ka | | Avg. kd | | Avg. KD | |
|---|---|---|---|---|---|---|---|
| Protein name | Antigen | (1/Ms) | 95% CI | (1/s) | 95% CI | (M) | 95% CI |
| PS3B1391 | cy PSMA | 1.57E+05 | 5.77E+04 | 1.96E−05 | 6.59E-06 | 1.26E−10 | 3.72E−11 |
| PS3B1391 | hu PSMA | 4.80E+05 | | 4.80E−05 | | 1.00E−10 | |
| PS3B1508 | cy PSMA | 1.46E+06 | | 3.23E−03 | | 2.20E−09 | |
| PS3B1508 | hu PSMA | 2.32E+05 | | 8.89E−05 | | 3.82E−10 | |
| PS3B1396 | cy PSMA | 5.87E+05 | | 1.90E−03 | | 5.07E−09 | |
| PS3B1396 | hu PSMA | 2.24E+05 | | 8.82E−05 | | 3.95E−10 | |
| PS3B1505 | cy PSMA | 4.29E+05 | | 1.24E−03 | | 5.01E−09 | |
| PS3B1505 | hu PSMA | 2.32E+05 | | 9.10E−05 | | 3.93E−10 | |

*Data in row 1 is an average of 3. Data in other rows an average of 2.

### Example 7.2: Thermal Stability of Bispecific Anti-PSMAxCD3 Antibodies

The thermal stability (conformational stability information including, Tm and Tagg) of selected anti-PSMAxCD3 antibodies was determined by nanoDSF method using a Prometheus instrument. Briefly, measurements were made by loading sample into 24 well capillary from a 384 well sample plate. Duplicate runs were performed. The thermal scans span from 20° C. to 95° C. at a rate of 1.0° C./minute. The data was processed to obtain integrated data and first derivation analysis for 330 nm, 350 nm, Ratio 330/350, and scatter data from which thermal transitions, onset of unfolding, Tm and Tagg were obtained and summarized in Table 30.

TABLE 30
Thermal stability data for bispecific anti-PSMA × CD3
antibodies as obtainedusing a nano DSF instrument.

| Name | T_{agg} | Tₘ1 | Tₘ2 | Tₘ3 |
|---|---|---|---|---|
| PS3B917 | 68.2° C. | 63.5° C. | 68.8º C. | |
| PS3B918 | 67.8º C. | 63.4° C. | 68.2° C. | |
| PS3B913 | 75.1° C. | 63.4° C. | 76.3º C. | |
| PS3B915 | 69.2º C. | 63.5° C. | | |
| PS3B914 | 64.5° C. | 61.9º C. | 67.0° C. | 75.8° C. |
| PS3B916 | 59.1° C. | 59.9º C. | | |
| PS3B919 | 81.0° C. | 63.5° C. | 68.3º C. | 85.7° C. |
| PS3B921 | 78.9º C. | 63.5° C. | 80.8° C. | |
| PS3B920 | 82.6° C. | 63.4º C. | 87.5° C. | |
| PS3B922 | 80.5° C. | 63.3° C. | 83.4° C. | |
| PS3B912 | 77.1° C. | 63.6° C. | 78.4º C. | |
| PS3B930 | 72.2° C. | 70.1º C. | | |
| PS3B931 | 72.2° C. | 69.5° C. | | |
| PS3B926 | 75.6° C. | 70.2° C. | 75.6° C. | |
| PS3B928 | 72.2° C. | 69.1° C. | | |
| PS3B927 | 69.1° C. | 66.5° C. | 69.4º C | |
| PS3B929 | 69.1º C. | 58.0° C. | 68.0° C. | |
| PS3B932 | 82.7° C. | 70.4° C. | 85.8° C. | |
| PS3B934 | 79.3º C. | 70.4° C. | 80.9º C. | |
| PS3B933 | 83.7º C. | 70.0° C. | 87.8° C. | |
| PS3B935 | 81.5° C. | 70.2° C. | 83.7º C. | |
| PS3B925 | 77.4° C. | 70.3º C. | 77.9º C. | |

### Example 7.3: Binding of Bispecific PSMAxCD3 Antibodies on PSMA+ Cells

Selected bispecific PSMAxCD3 antibodies were assessed for their ability to bind prostate cancer cell lines expressing PSMA.

22RV1 and C4-2B cells were plated at 50,000 cells per well in 50 μl of assay medium (RPMI, 10% HI FBS) in V bottom plates. Serial dilutions of antibodies were prepared in assay medium with 50 μl of antibody dilutions added to the plates containing cells. The plates were incubated for 60 min at 37° C. at which time 100 μl of staining buffer (Becton Dickinson Cat #554657) was added to all wells of each plate. The plates were centrifuged at 300×G for 5 minutes and the medium was removed from the wells. 200 μl of staining buffer was added to all wells of each plate. The plates were centrifuged at 300×G for 5 minutes and the medium was removed from the wells. 50 μl of 2 μg/ml AlexaFluor647-labeled goat anti-human Fc was added to all wells of the plates and the plates were incubated for 30 minutes at 4° C. 150 μl of staining buffer was added to all wells of each plate. The plates were centrifuged at 300×G for 5 minutes and the medium was removed from the wells. Two hundred microliters of running buffer (Staining buffer plus 1 mM EDTA, 0.1% pluronic acid was added to all wells of the plates. The plates were centrifuged at 300×G for 5 minutes and the medium was removed from the wells. Thirty microliters running buffer was added to all wells with cells and the plates were analyzed on the IQUE Plus instrument (Sartorius). Briefly, cells were gated on a FCS vs. SSC gate to eliminate cellular debris, and then the cell populations were gated on singlet cells. Antibody binding was assessed in the red laser channel. Signal (Mab plus secondary antibody) to background (secondary antibody only) ratios were calculated for each plate and the resultant data was plotted vs. bispecific antibody concentration in GeneData Screener using 4 parameter curve fitting to generate EC50 values, summarized in Table 31.

TABLE 31
EC50 values of bispecific PSMA × CD3 antibodies binding
to PSMA-expressing cell lines in flow cytometry assays.

| | 22RV1 binding | C4-2B binding |
|---|---|---|
| Name | EC50 [M] | EC50 [M] |
| PS3B917 | 7.50E−10 | 1.90E−09 |
| PS3B918 | 6.00E−08 | 6.00E−08 |
| PS3B913 | 3.42E−09 | 4.56E−09 |
| PS3B915 | 6.00E−08 | 6.00E−08 |
| PS3B914 | 6.00E−08 | 6.00E−08 |
| PS3B916 | 6.00E−08 | 6.00E−08 |
| PS3B919 | 2.49E−08 | 2.33E−08 |
| PS3B921 | 4.03E−09 | 6.44E−09 |
| PS3B920 | 6.00E−08 | 6.00E−08 |
| PS3B922 | 3.47E−08 | 4.41E−08 |
| PS3B912 | 5.40E−08 | 3.68E−08 |
| PS3B930 | 1.06E−09 | 3.01E−09 |
| PS3B931 | 6.00E−08 | 6.00E−08 |
| PS3B926 | 2.90E−09 | 4.55E−09 |
| PS3B928 | 6.00E−08 | 6.00E−08 |
| PS3B927 | 6.00E−08 | 6.00E−08 |
| PS3B929 | 5.48E−08 | 6.00E−08 |
| PS3B932 | 4.87E−08 | 6.00E−08 |
| PS3B934 | 4.96E−09 | 1.10E−08 |
| PS3B933 | 6.00E−08 | 6.00E−08 |
| PS3B935 | 3.95E−08 | 8.22E−09 |
| PS3B925 | 2.12E−08 | 1.17E−08 |

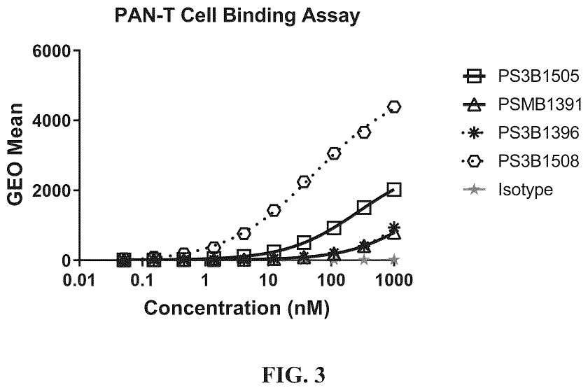

Binding of anti-PSMA/CD3 bispecific on PAN-T cells via Flow. Human PAN-T Cells (Biological Specialty Corporation, Colmar, PA) were thawed and transferred to a 15 mL conical with DPBS. The cells were centrifuged 1300 rpm for 5 minutes. DPBS was aspirated and cells were re-suspended in DPBS. The cells were counted using the Vi-cell XR cell viability analyzer and were plated at 100K/well in 100 uL DPBS. The plate was centrifuged 1200 rpm for 3 minutes and washed 2× with DPBS. Cells were stained with Violet Live/Dead stain (Thermo-Fisher) and incubated at RT in the dark for 25 min. The cells were centrifuged and washed 2× with FACS staining buffer (BD Pharmingen). Test antibodies were diluted to a final starting concentration of 1 μM in FACS staining buffer and 3-fold serial dilutions were prepared from the starting concentration for a total of 10 dilution points. The serially diluted test antibodies (100 μL/well) were added to the cells and incubated for 30 min at 37° C. Cells were washed 2× with FACS staining buffer and AlexaFluor 647-conjugated Donkey anti-human secondary antibody (Jackson Immunoresearch) was added and allowed to incubate with the cells for 30 min at 4° C. Cells were washed 2× with FACS staining buffer and re-suspended in 100 μL FACS Buffer. Cells were run on BD Celesta using FACS Diva software and analyzed using FLOWJO. FIG. 3 shows that the PSMA/CD3 bispecific antibodies display differential CD3 cell binding profiles detected by flow cytometry.

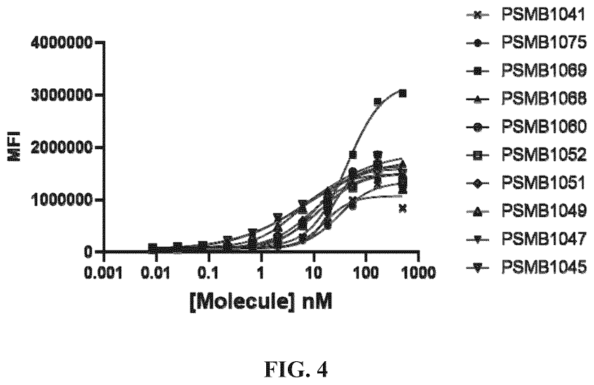

Binding curves demonstrated in Table 32 below and FIG. 4 were generated against the prostate cell line C4-2B at 37° C. in RPMI media plus 10% fetal bovine serum after 1 hr incubation. Molecule concentrations ranged from 500-0 nM over 12-points at 3-fold dilution. Selective binding to PSMA was validated using an isotype control. Values reported in the Table 32 were generated by fitting the data to a four-parameter function for ligand binding generating values for y-min, y-max, EC50, and Hill. EC90 were calculated using the equation EC90=(90−(100−90))\hat{ }(1/Hill)*EC50. All curves exhibited a similar Y-min with an average of 7.1+/−1.3E+4 for all curves. None of the Y-min values deviated significantly from the average value. The average fitted Y-max value was 1.7+/−0.6E+6. Molecule PSMB1069 exhibited a 2-fold higher binding signal from the average. None of the other molecules exhibited a significant difference from the average. These molecules exhibited an average EC50=17+/−12 nM.

TABLE 32
EC50 values of anti-PSMA antibodies binding
to PSMA-expressing cell line C4-2B
measured by flow cytometry assays.

| | | | EC50 | EC90 |
|---|---|---|---|---|
| Molecule | Y-min | Y-max | (nM) | (nM) |
| PSMB1041 | 7.2E+04 | 1.1E+06 | 16 | 59 |
| PSMB1045 | 5.3E+04 | 1.8E+06 | 7 | 248 |
| PSMB1047 | 7.9E+04 | 1.6E+06 | 22 | 71 |
| PSMB1049 | 8.4E+04 | 1.5E+06 | 5 | 40 |
| PSMB1051 | 6.7E+04 | 1.5E+06 | 10 | 76 |
| PSMB1052 | 6.8E+04 | 1.5E+06 | 13 | 100 |
| PSMB1060 | 8.6E+04 | 1.6E+06 | 11 | 59 |
| PSMB1068 | 4.8E+04 | 1.9E+06 | 8 | 247 |
| PSMB1069 | 8.7E+04 | 3.3E+06 | 44 | 254 |
| PSMB1075 | 6.6E+04 | 1.3E+06 | 30 | 156 |

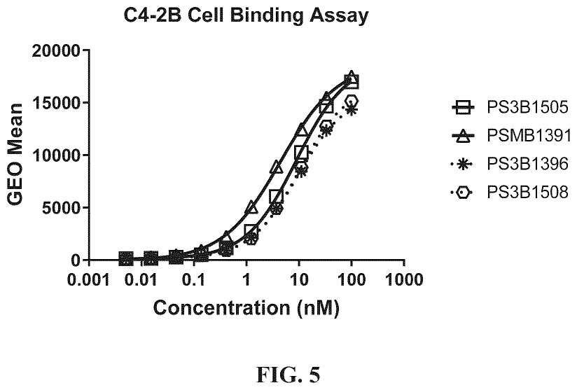

Binding of anti-PSMA variants/CD3 bispecific on T cells via flow cytometry. C4-2B human prostate tumor cells were washed with DPBS and 0.25% trypsin was added to allow cells to detach. Media was added to neutralize trypsin and the cells were transferred to a 15 mL conical with DPBS. The cells were centrifuged 1300 rpm for 5 minutes. DPBS was aspirated and cells were re-suspended in DPBS. The cells were counted using the Vi-cell XR cell viability analyzer and were plated at 100K/well in 100 μL DPBS. The plate was centrifuged 1200 rpm for 3 minutes and washed 2× with DPBS. Cells were stained with Violet Live/Dead stain (Thermo-Fisher) and incubated at RT in the dark for 25 min. The cells were centrifuged and washed 2× with FACS staining buffer (BD Pharmingen). Test antibodies were diluted to a final starting concentration of 100 nM in FACS staining buffer and 3-fold serial dilutions were prepared from the starting concentration for a total of 10 dilution points. The serially diluted test antibodies (100 μL/well) were added to the cells and incubated for 30 min at 37° C. Cells were washed 2× with FACS staining buffer and AlexaFluor 647-conjugated Donkey anti-human secondary antibody (Jackson Immunoresearch) was added and allowed to incubate with the cells for 30 min at 4° C. Cells were washed 2× with FACS staining buffer and re-suspended in 100 μL FACS Buffer. Cells were run on BD CELESTA using FACS Diva software and analyzed using FLOWJO. FIG. 5 shows that PSMA/CD3 bispecific antibodies display similar PSMA cell binding profiles detected by flow cytometry.

### Example 7.4: Internalization of PSMA

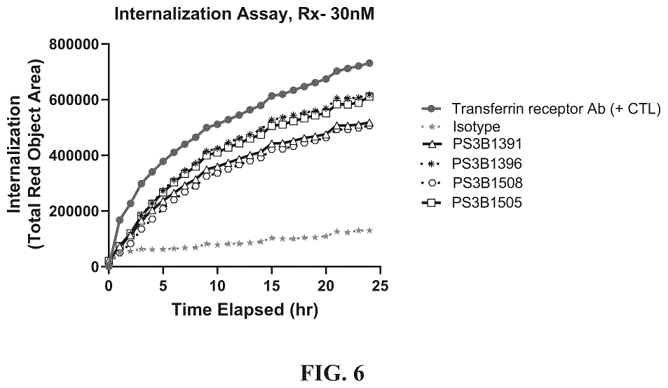

C4-2B human prostate tumor cells were washed with DPBS and 0.25% trypsin was added to allow cells to detach. Media was added to neutralize trypsin and the cells were transferred to a 15 mL conical with DPBS. The cells were centrifuged 1300 rpm for 5 minutes. DPBS was aspirated and cells were re-suspended in DPBS. The cells were counted using the Vi-cell XR cell viability analyzer and were plated at 40K/well in 50 μL Phenol Red-Free PRMI+10% HI FBS. The PSMA/CD3 bispecific or control antibodies were incubated with IncuCyte® Human Fab-fluor-pH Red Antibody labeling dye for 15 minutes then 50 μL of conjugated PSMA/CD3:Fab-fluor-pH Red complex was added to the wells containing C4-2B cells. The plates were placed in an IncuCyte S3® (Essen) at 37° C. with 5% CO2 for 24 hours. The Ab:Fab-fluor complex that is internalized by the target cells is processed by acidic lysosomes, which produces the red fluorogenic signal that is captured and analyzed by the IncuCyte®. FIG. 6 shows that PSMA/CD3 bispecific antibodies internalize but at a lesser rate than the transferrin receptor.

### Example 7.5: T-Cell Mediated Killing of Bispecific PSMAxCD3 Antibodies on PSMA+ Cells Via Flow Cytometry

Selected bispecific PSMA×CD3 antibodies were assessed for their ability to mediate T cell mediated killing of prostate cancer cells.

T cell mediated killing of the PSMA×CD3 bispecific antibodies was measured using an assay that indirectly measures cell killing via flow cytometry. Target cell population are identified base on cell viability. Test samples and controls were prepared at 20 nM in assay medium (10% RPMI, 10% HI FCS). Half log serial dilutions for an 11-point titration of compounds in sterile polypropylene plates were prepared. Additional wells were used for controls without compounds, T cells or tumor cell containing wells only in assay medium. C4-2B cells were harvested from the cell culture flasks and cells were resuspended in PBS. Cells were stained with 20 pM CFSE for 10 minutes at room temperature. 25 mL of HI FBS was added to stop the staining reaction and the cells were centrifuged at 300×G for 5 minutes. Cells were diluted to 1×10⁶/ml and then plated as tumor target cells in 50 μL assay medium for 50,000 cells/well into a V-bottom tissue culture treated polystyrene assay plate. 50 μL/well of assay media was added to the control wells that did not receive tumor cells. Human PAN-T cell vials were thawed in a water bath set at 37° C. and washed twice by adding 10 ml assay medium and centrifuging at 400×G for 5 minutes. T cells were resuspended to 1×10⁶/mL in assay medium and 50 μL containing 50,000/well were added to the assay plates containing tumor target cells. 50 μL/well assay media was added to the control wells that did not receive tumor cells. 100 μL/well of serially diluted antibodies were added to the assay plates containing cell mixture of target and effector cells. Plates were incubated at 37° C., 5% CO₂ in a humidified cell culture incubator for 72 hours.

Following the incubation assay plates were centrifuged at 500×G for 5 minutes and medium was removed from the wells. 150 μL DPBS was added to each well and the plates were centrifuged at 500×G for 5 minutes and medium was removed from the wells. The cultures were assessed using flow cytometry on the INTELLICYT IQ Plus for viable tumor cells using near IR live/dead stain. T cell activation was assessed using a brilliant violet-labeled anti-CD25 MAB. Cells were gated in an FSC vs SSC gate to eliminate debris. Tumor cells were identified as CFSE positive cells. T cells were identified as CSFE negative cells. Tumor cell viability was calculated as number of live/dead stain positive tumor cells as a percentage of total CSFE cells. Activated T cells were calculated as the number of CD25 positive cells as a percentage of the total number of the live CFSE negative population. The data for percent dead tumor cells and activated T cells were plotted vs. antibody concentration in Gene Data Screener using 4 parameter curve fitting to generate EC50 values. Table 33 shows EC50 values for T cell activation and tumor cell killing.

TABLE 33
In vitro T cell mediated killing of tumor cells and T cell
activation by bispecific PSMA × CD3 antibodies.

| | Tumor killing | T cell activation |
|---|---|---|
| Name | EC50 [M] | EC50 [M] |
| PS3B917 | 7.31E−12 | 4.48E−12 |
| PS3B918 | 1.00E−08 | 1.00E−08 |
| PS3B913 | 3.11E−10 | 1.55E−10 |
| PS3B915 | 1.00E−08 | 1.00E−08 |
| PS3B914 | 1.00E−08 | 1.00E−08 |
| PS3B916 | 1.00E−08 | 1.00E−08 |
| PS3B919 | 2.10E−10 | 1.87E−10 |
| PS3B921 | 3.11E−11 | 2.11E−11 |
| PS3B920 | 3.20E−09 | 1.77E−09 |
| PS3B922 | 7.79E−11 | 5.85E−11 |
| PS3B912 | 6.51E−11 | 4.73E−11 |
| PS3B930 | 4.83E−12 | 2.40E−12 |
| PS3B931 | 1.00E−08 | 1.00E−08 |
| PS3B926 | 4.78E−11 | 3.45E−11 |
| PS3B928 | 4.81E−10 | 1.84E−10 |
| PS3B927 | 1.00E−08 | 1.00E−08 |
| PS3B929 | 1.00E−08 | 1.00E−08 |
| PS3B932 | 6.42E−12 | 1.20E−11 |
| PS3B934 | 7.24E−12 | 4.81E−12 |
| PS3B933 | 4.76E−11 | 6.01E−11 |
| PS3B935 | 7.16E−12 | 6.72E−12 |
| PS3B925 | 7.98E−12 | 4.59E−12 |

### Example 7.6: T-Cell Mediated Killing of Bispecific PSMAxCD3 Antibodies on PSMA+ Cells Via Incucyte

Select bispecific PSMAxCD3 antibodies were assessed for their ability to mediate T cell mediated killing of prostate cancer cells via IncuCyte®-based cytotoxic assay.

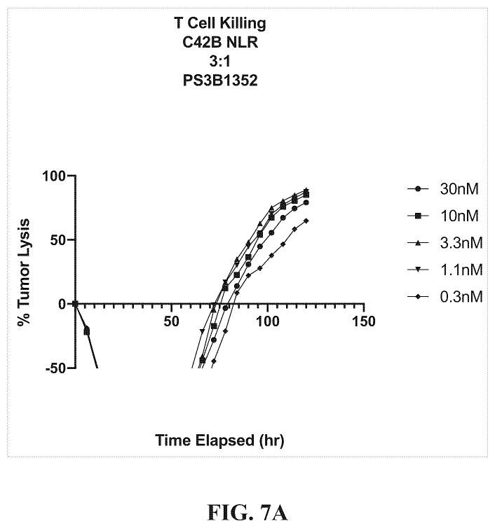

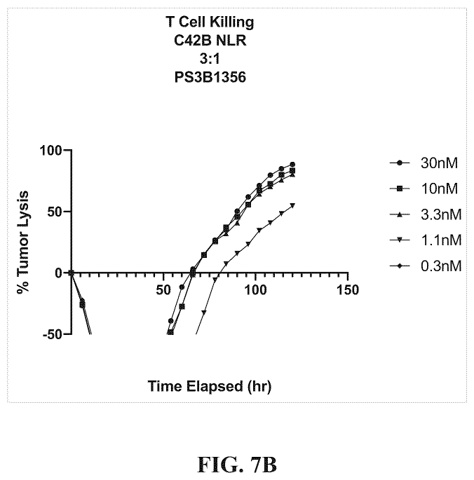

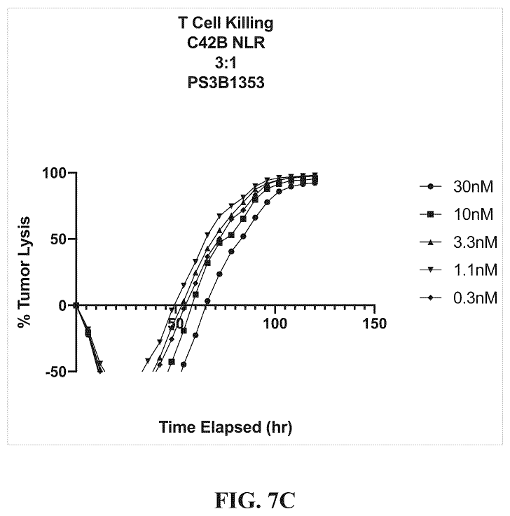

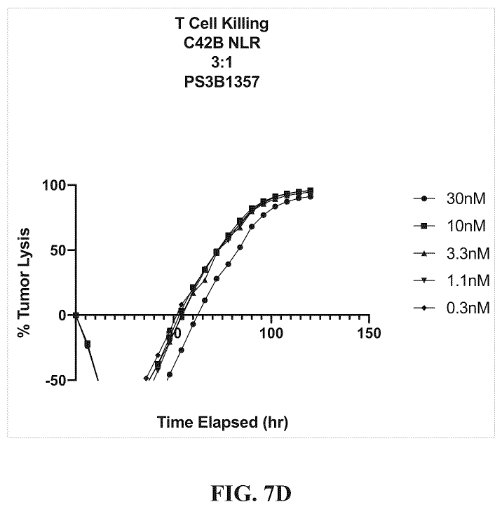

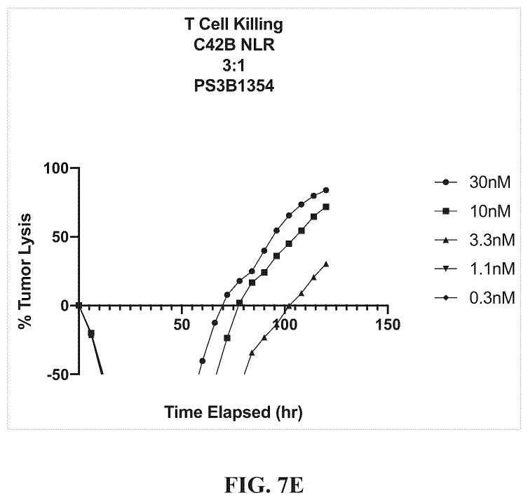

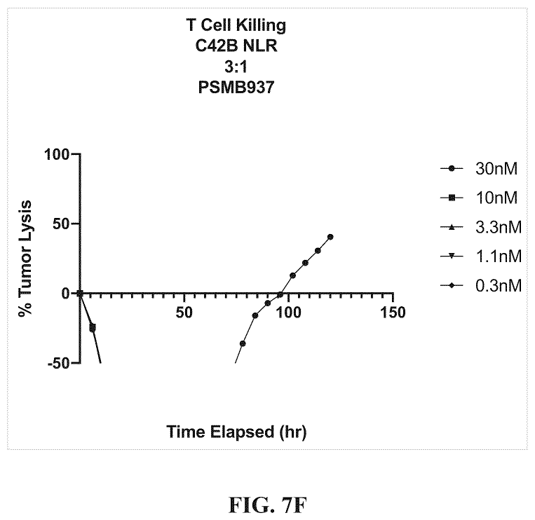

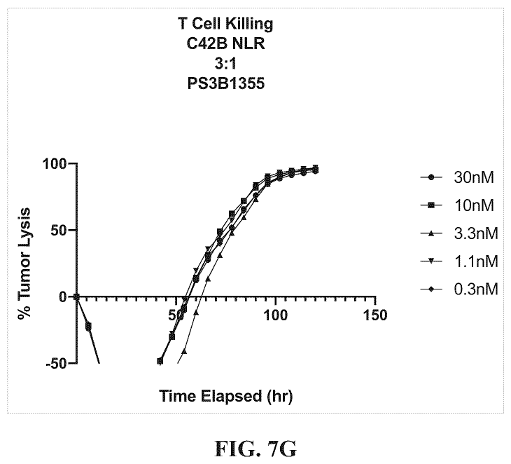

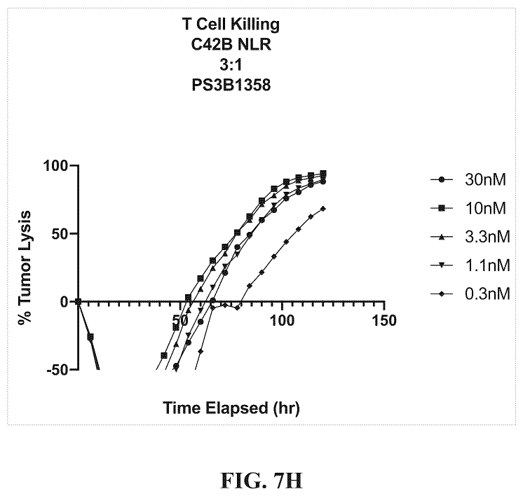

Healthy donor T cells. PSMA+ C4-2B cells stably expressing red nuclear dye were generated to be used in the IncuCyte-based cytotoxicity assay. Frozen vials of healthy donor T cells (Biological Specialty Corporation, Colmar, PA) were thawed in a 37° C. water bath, transferred to a 15 mL conical tube, and washed once with 5 mL phenol-red-free RPMI/10% HI FBS medium. The cells were counted using the VIACELL XR cell viability analyzer and the T cells were combined with target cells for a final effector T cell to target cell (E:T) ratio of 3:1. The cell mixture was combined in a 50 mL conical tube. The cell mixture (100 μL/well) was added to a clear 96-well flat-bottom plate. Next, the test antibodies were diluted to a final starting concentration of 60 nM in phenol-red-free RPMI/10% HI FBS medium and 3-fold serial dilutions were prepared from the starting concentration for a total of 11 dilution points. The serially diluted test antibodies (100 μL/well) were added to the combined cells. The plates were placed in either an IncuCyte® Zoom or an IncuCyte 53® (Essen) at 37° C. with 5% CO₂ for 120 hours. The target cell lines stably express red nuclear dye, which is used to track the kinetics of target cell lysis. Percent cell growth inhibition (%)=(Initial viable target cell number−Current viable target cell number)/Initial viable cell number*100%. Table 34 and FIGS. 7A-7H show cytotoxicity for C4-2B cells with increasing concentrations of anti-PSMA. Isolated PAN-T cells were co-incubated with PSMA+ C4-2B cells in the presence of bispecific PSMA/T cell redirection antibodies for 120 hours.

TABLE 34
Bispecific anti-PSMA/anti-T cell
redirection antibodies evaluated in an
IncuCyte ®-based cytotoxicity assay.

| | Cytotoxicity (C4-2B cells, | | | | |
|---|---|---|---|---|---|
| | 3:1 E:T ratio, 5 Day) | | | | |
| | 30 | 10 | 3.3 | 1.1 | 0.3 |
| Name | nM | nM | nM | nM | nM |
| PS3B1352 | 79% | 85% | 89% | 88% | 65% |
| PS3B1356 | 88% | 83% | 80% | 55% | No |
| | | | | | lysis |
| PS3B1353 | 92% | 95% | 97% | 98% | 98% |
| PS3B1357 | 91% | 96% | 95% | 96% | 96% |
| PS3B1354 | 84% | 72% | 30% | No | No |
| | | | | lysis | lysis |
| PSMB937 | 41% | No | No | No | No |
| | | lysis | lysis | lysis | lysis |
| PS3B1355 | 94% | 95% | 96% | 97% | 92% |
| PS3B1358 | 88% | 94% | 93% | 90% | 68% |

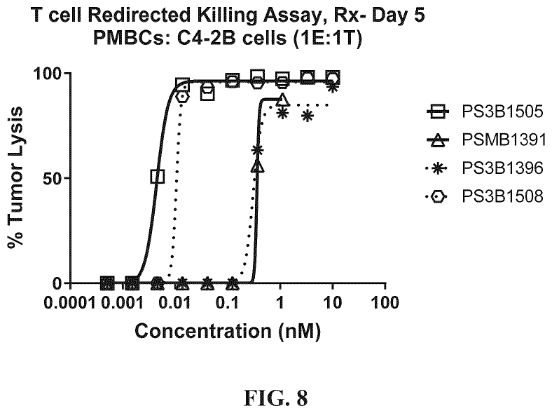

Healthy PBMCs. PSMA+ C4-2B human prostate tumor cells expressing red nuclear dye were generated to be used in the IncuCyte®-based cytotoxicity assay. Frozen vials of healthy PBMCs (Hemacare, Los Angeles, CA) were thawed in a 37° C. water bath, transferred to a 15 mL conical tube, and washed once with 5 mL phenol-red-free RPMI/10% HI FBS medium. The cells were counted using the VIACELL XR cell viability analyzer and the PBMCs were combined with target cells for a final PBMC to target cell (E:T) ratio of 1:1. The cell mixture was combined in a 50 mL conical tube. The cell mixture (100 μL/well) was added to a clear 96-well flat-bottom plate. Next, the test antibodies were diluted to a final starting concentration of 30 nM in phenol-red-free RPMI/10% HI FBS medium and 3-fold serial dilutions were prepared from the starting concentration for a total of 11 dilution points. The serially diluted test antibodies (100 μL/well) were added to the combined cells. The plates were placed in either an IncuCyte® Zoom or an IncuCyte S3® (Essen) at 37° C. with 5% CO₂ for 120 hours. The target cell lines stably express red nuclear dye, which is used to track the kinetics of target cell lysis. Percent cell growth inhibition (%)=(Initial viable target cell number−Current viable target cell number)/Initial viable cell number*100%. FIG. 8 shows that the PSMA/CD3 bispecific antibodies induce differential C4-2B cytotoxic effects.

### Example 7.7: Evaluating Cytokine Induction by Bispecific Anti-PSMAxCD3 Antibodies

Select bispecific PSMAxCD3 antibodies were assessed for their ability to induce cytokine release.

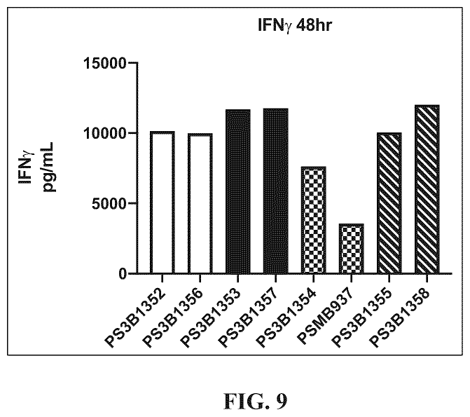

Supernatants collected from the in-vitro cytotoxicity experiment described above were analyzed using the Human Proinflammatory Panel I tissue culture kit (Meso Scale Discovery). Supernatants were thawed on wet ice, spun at 1,500 rpm for 5 minutes at 4° C., then placed on ice. The MULT-SPOT assay plates were pre-washed per the manufacturer's protocol. A standard curve was prepared by serial dilution of the provided calibrators in MSD Diluent 1. The standards and test antibody samples (25 μL/well) were added to the pre-washed plates. Subsequent incubations and washes were all carried out per manufacturer's protocol. Assay plates were read on the SECTOR Imager 6000. IFNγ concentrations were quantified for each PSMAxCD3 bispecific antibody evaluated. FIG. 9 shows functional cytokine release by T cells activated by PSMAxCD3 antibodies.

### Example 7.8: T-Cell Mediated Killing of Bispecific PSMAxCD3 Antibodies on PSMA+ Cells Via xCelligence

Select bispecific PSMAxCD3 antibodies were assessed for their ability to mediate T cell mediated killing of prostate cancer cells, C4-2B. C4-2B, a prostate cancer cell line expressing 150,000 PSMA/cell was used at a 3:1 Effector to Target ratio (E:T), using three PAN-T donors. On day 0 of the experiment, xCelligence plates were blanked with 50 μl of growth media. Plates were then seeded with 20,000 C4-2B (50 μL out of 0.4×10⁶ cells/ml) cells per well. Plates were then incubated on the xCelligence machine overnight. On day 1 of the experiment, three PAN-T donors were used to prepare the E:T ratio by adding 50 μL of 1.2×10⁶ cells/mL (60,000 cells). Then 50 μL of the appropriate bispecific antibodies were added to the appropriate wells for each plate. CD3×null was used as a control. Tumor/target only wells were assigned to be used for normalization in the percent cytolysis calculation. Final antibody concentrations were 50 nM, 10 nM, 2 nM, 0.4 nM, 80 pM and 0 nM. Plates were then placed in the XCELLIGENCE machine and impedance was recorded every 15 minutes for 120 hours. Percent cytolysis was calculated on the RTCA software using the equation % cytolysis=[1−(NCI)/(AvgNCIR)]×100, where NCI is the average cell index of the well and AvgNCIR is the average cell index of the tumor only reference wells. Table 35 summarizes cytolysis for each PSMA×CD3 bispecific molecule over time.

TABLE 35
Summary of % cytolysis at time point 120 hours for all four bispecific
antibodies, for three PAN-T donors, at each dose concentration.

| Donor ID | Name | 50 nM | 10 nM | 2 nM | 0.4 nM | 80 pM |
|---|---|---|---|---|---|---|
| #1 | PS3B1352 | 100% | 100% | 100% | 100% | 77% |
| | PS3B1353 | 100% | 100% | 100% | 100% | 100% |
| | PS3B1356 | 100% | 100% | 100% | 100% | 0% |
| | PS3B1357 | 100% | 100% | 100% | 100% | 100% |
| #2 | PS3B1352 | 100% | 100% | 100% | 100% | 0% |
| | PS3B1353 | 100% | 100% | 100% | 100% | 100% |
| | PS3B1356 | 100% | 100% | 100% | 24% | 0% |
| | PS3B1357 | 100% | 100% | 100% | 100% | 100% |
| #3 | PS3B1352 | 100% | 100% | 100% | 100% | 9% |
| | PS3B1353 | 100% | 100% | 100% | 100% | 100% |
| | PS3B1356 | 100% | 100% | 100% | 100% | 0% |
| | PS3B1357 | 100% | 100% | 100% | 100% | 100% |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated antibody that binds to PSMA comprising:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
(8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
(9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
(10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
(11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372;
(12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406;
(13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
(14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
(15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
(16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542;
(17) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576;
(18) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
(19) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
(20) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678;
(21) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
(22) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746;
(23) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780; or
(24) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814.

2. The isolated antibody of claim 1, wherein the VH CDR1, VH CDR2, VH CDR3, VL CDR1, VL CDR2, and VL CDR3 amino acid sequences are according to the
(i) Kabat numbering system;
(ii) Chothia numbering system;
(iii) AbM numbering system;
(iv) Contact numbering system;
(v) IMGT numbering system or a combination thereof.

3. The isolated antibody of claim 1, further comprising a VH having an amino acid sequence at least 80% identical to the amino acid sequence of SEQ ID NOs:31, 99, 167, 235, 303, 371, 405, 439, 473, 507, 541, 575, 643, 677, 711, 779 or 813 and a VL having an amino acid sequence at least 80% identical to the amino acid sequence of SEQ ID NOs:32, 66, 100, 134, 236, 270, 372, 406, 474, 508, 542, 576, 678, 746, 780 or 814.

4. The isolated antibody of claim 1, further comprising a heavy chain (HC) having an amino acid sequence at least 80% identical to the amino acid sequence of SEQ ID NOs:33, 101, 169, 237, 305, 373, 407 or 441 and a light chain (LC) having an amino acid sequence at least 80% identical to the amino acid sequence of SEQ ID NOs:34, 68, 102, 136, 238, 272, 374 or 408.

5. An isolated antibody that binds PSMA comprising
a. (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
b. (i) a VH comprising a VH CDR1, a VH CDR2, and a VH CDR3 having an amino acid sequence of SEQ ID NOs:205, 206, and 411, respectively, and (ii) a VL comprising a VL CDR1, VL CDR2, and VL CDR3 having an amino acid sequence of SEQ ID NOs:242, 209, and 244, respectively;
c. (i) a VH having an amino acid sequence of SEQ ID NO:439; and (ii) a VL having an amino acid sequence of SEQ ID NO:270; or
d. (i) a HC having an amino acid sequence of SEQ ID NO:441; and (ii) a LC having an amino acid sequence of SEQ ID NO:272.

6. The isolated antibody of claim 1, wherein the antibody is a humanized antibody.

7. The isolated antibody of claim 1, wherein the antibody is a human antibody.

8. The isolated antibody of claim 1, wherein the binding domain that binds to PSMA is a scFv, an scFv dimer, a Fv, a Fab, a Fab, a F(ab)₂, a dsFv, a sdAb, a VHH or a single chain antibody.

9. The isolated antibody of claim 1, wherein the antibody is an IgG antibody.

10. The isolated antibody of claim 9, wherein the IgG antibody is an IgG1, IgG2, IgG3, or IgG4 antibody.

11. The isolated antibody of claim 1, wherein the antibody comprises a kappa light chain.

12. The isolated antibody of claim 1, wherein the antibody comprises a lambda light chain.

13. The isolated antibody of claim 1, wherein the antibody is a monoclonal antibody.

14. The isolated antibody of claim 1, wherein the antibody binds a PSMA antigen.

15. The isolated antibody of claim 1, wherein the antibody binds a PSMA epitope.

16. The isolated antibody of claim 1, wherein the antibody specifically binds to PSMA.

17. The isolated antibody of claim 1, wherein the VH CDR1, VH CDR2, VH CDR3, VL CDR1, VL CDR2 and VL CDR3 form a binding site for an antigen of the PSMA.

18. The isolated antibody of claim 1, wherein the VH CDR1, VH CDR2, VH CDR3, VL CDR1, VL CDR2 and VL CDR3 form a binding site for an epitope of the PSMA.

19. The isolated antibody of claim 1, wherein the PSMA is present on the surface of a cell.

20. The isolated antibody of claim 19, wherein the cell is a prostate cell.

21. The isolated antibody of claim 19, wherein the cell is a prostate cancer cell.

22. The isolated antibody of claim 19, wherein the cell is a renal cell.

23. The isolated antibody of claim 19, wherein the cell is a renal cancer cell.

24. The isolated antibody of claim 23, wherein the renal cancer is a metastatic renal cell carcinoma.

25. The isolated antibody of claim 1, wherein the antibody is multivalent.

26. The isolated antibody of claim 25, wherein the antibody is capable of binding at least three antigens.

27. The isolated antibody of claim 25, wherein the antibody is capable of binding at least four antigens.

28. The isolated antibody of claim 25, wherein the antibody is capable of binding at least five antigens.

29. The isolated antibody of claim 1, wherein the antibody is a multispecific antibody.

30. The isolated antibody of claim 29, wherein the antibody is a bispecific antibody.

31. The isolated antibody of claim 29, wherein the antibody is a trispecific antibody.

32. The isolated antibody of claim 29, wherein the antibody is a quadraspecific antibody.

33. A nucleic acid encoding the antibody of claim 1.

34. A vector comprising the nucleic acid of claim 33.

35. A host cell comprising the vector of claim 34.

36. A kit comprising the vector of claim 34 and packaging for the same.

37. A kit comprising the antibody of claim 1 and packaging for the same.

38. A pharmaceutical composition comprising the antibody of claim 1, and a pharmaceutically acceptable carrier.

39. A method of producing the pharmaceutical composition of claim 38, comprising combining the antibody with a pharmaceutically acceptable carrier to obtain the pharmaceutical composition.

40. An isolated multispecific antibody, comprising:
(a) a first binding domain that binds to PSMA, wherein the first binding domain comprises:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
(6) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134;
(7) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
(8) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
(9) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236;
(10) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
(11) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372;
(12) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406;
(13) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270;
(14) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
(15) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
(16) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542;
(17) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576;
(18) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100;
(19) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508;
(20) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678;
(21) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474;
(22) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746;
(23) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780; or
(24) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814; and
(b) a second binding domain that binds to CD3, wherein the second binding domain comprises:
(1) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1505; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1464;
(2) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916;
(3) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984;
(4) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1463; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1464;
(5) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848; or
(6) a scFv comprising a VH CDR1, a VH CDR2, a VH CDR3, a VL CDR1, a VL CDR2, a VL CDR3 having an amino acid sequence of a VH CDR1, a VH CDR2, a VH CDR3, a VL CDR1, a VL CDR2, a VL CDR3 respectively, of a scFv having an amino acid sequence of SEQ ID NO:1524.

41. The isolated multispecific antibody of claim 40, wherein the VH CDR1, VH CDR2, VH CDR3, VL CDR1, VL CDR2, and VL CDR3 amino acid sequences are according to the
(i) Kabat numbering system;
(ii) Chothia numbering system;
(iii) AbM numbering system;
(iv) Contact numbering system;
(v) IMGT numbering system or a combination thereof.

42. The isolated multispecific antibody of claim 40, wherein the first binding domain that binds PSMA comprises a heavy chain (HC) having an amino acid sequence at least 80% identical to the amino acid sequence of SEQ ID NOs:33, 101, 169, 237, 305, 373, 407, 441, 1242, 1244, 1248, 1250, 1252, 1254, 1256, 1258, 1260, 1262, 1264, 1266, 1268 or 1270 and/or a light chain (LC) having an amino acid sequence at least 80% identical to the amino acid sequence of SEQ ID NOs:34, 68, 102, 136, 238, 272, 374 or 408.

43. The isolated multispecific antibody of claim 40, wherein the first binding domain that binds PSMA comprises a scFv having an amino acid sequence at least 80% identical to the amino acid sequence of any one of SEQ ID NOs:1485-1500 or SEQ ID NOs:1526-1531.

44. The isolated multispecific antibody of claim 40, wherein the first binding domain that binds PSMA comprises (i) a HC having an amino acid sequence of SEQ ID NO:441; and (ii) a LC having an amino acid sequence of SEQ ID NO:272.

45. The isolated multispecific antibody of claim 40, wherein the second binding domain that binds CD3 comprises a heavy chain (HC) having an amino acid sequence at least 80% identical to the amino acid sequence of SEQ ID NOs:849, 883, 917, 951, 985, 1019, 1504, 1455, 1192, 1194, 1167, 1218 or 1238 and/or a light chain (LC) having an amino acid sequence at least 80% identical to the amino acid sequence of SEQ ID NOs:850, 918, 986, 1193, 1195 or 1219.

46. The isolated multispecific antibody of claim 40, wherein the second binding domain that binds CD3 comprises a scFv having an amino acid sequence at least 80% identical to the amino acid sequence of SEQ ID NOs:1186, 1187, 1523 or 1524.

47. An isolated bispecific antibody comprising a first binding domain that binds PSMA and a second binding domain that binds CD3, wherein
a. the first binding domain that binds PSMA comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270, and the second binding domain that binds CD3 comprises a scFv comprising a VH CDR1, a VH CDR2, a VH CDR3, a VL CDR1, a VL CDR2, and a VL CDR3 of a scFv having an amino acid sequence of SEQ ID NO:1524;
b. the first binding domain that binds PSMA comprises a VH CDR1, VH CDR2, VH CDR3, VL CDR1, VL CDR2, VL CDR3 of SEQ ID NO:205, 206, 411, 242, 209 and 244, respectively, wherein the amino acid sequences are according to the Kabat numbering system; and the second binding domain that binds CD3 comprises a VH CDR1, VH CDR2, VH CDR3, VL CDR1, VL CDR2, VL CDR3 of SEQ ID NO:1467, 1468, 1506, 1470, 1471 and 1472, respectively; wherein the amino acid sequences are according to the Kabat numbering system;
c. the first binding domain that binds PSMA comprises (i) a VH having an amino acid sequence of SEQ ID NO:439; and (ii) a VL having an amino acid sequence of SEQ ID NO:270, and the second binding domain that binds CD3 comprises a scFv of SEQ ID NO:1524, respectively; and/or
d. the first binding domain that binds PSMA comprises (i) a HC2 having an amino acid sequence of SEQ ID NO:441; and (ii) a LC2 having an amino acid sequence of SEQ ID NO:272, respectively; and the second binding domain that binds CD3 comprises a HC1 of SEQ ID NO:1455.

48. The isolated multispecific antibody of claim 40, wherein the antibody is a humanized antibody.

49. The isolated multispecific antibody of claim 40, wherein the antibody is a human antibody.

50. The isolated multispecific antibody of claim 40, wherein the first binding domain, the second binding domain and/or the first and second is a scFv, an scFv dimer, a Fv, a Fab, a Fab, a F(ab′)₂, a dsFv, a sdAb, a VHH or a single chain antibody.

51. The isolated multispecific antibody of claim 40, wherein the antibody is an IgG antibody.

52. The isolated multispecific antibody of claim 51, wherein the IgG antibody is an IgG1, IgG2, IgG3, or IgG4 antibody.

53. The isolated multispecific antibody of claim 40, wherein the antibody comprises a kappa light chain.

54. The isolated multispecific antibody of claim 40, wherein the antibody comprises a lambda light chain.

55. The isolated multispecific antibody of claim 40, wherein the antibody is a monoclonal antibody.

56. The isolated multispecific antibody of claim 40, wherein the first binding domain binds a PSMA antigen.

57. The isolated multispecific antibody of claim 40, wherein first binding domain binds a PSMA epitope.

58. The isolated multispecific antibody of claim 40, wherein the first binding domain specifically binds to PSMA.

59. The isolated multispecific antibody of claim 40, wherein the VH CDR1, VH CDR2, VH CDR3, VL CDR1, VL CDR2 and VL CDR3 of the first binding domain form a binding site for an antigen of the PSMA.

60. The isolated multispecific antibody of claim 40, wherein the VH CDR1, VH CDR2, VH CDR3, VL CDR1, VL CDR2 and VL CDR3 of the first binding domain form a binding site for an epitope of the PSMA.

61. The isolated multispecific antibody of claim 40, wherein the second binding domain binds a CD3 antigen.

62. The isolated multispecific antibody of claim 40, wherein the second binding domain binds a CD3 epitope.

63. The isolated multispecific antibody of claim 40, wherein the second binding domain specifically binds to CD3.

64. The isolated multispecific antibody of claim 40, wherein the VH CDR1, VH CDR2, VH CDR3, VL CDR1, VL CDR2 and VL CDR3 of the second binding domain form a binding site for an antigen of the CD3.

65. The isolated multispecific antibody of claim 40, wherein the VH CDR1, VH CDR2, VH CDR3, VL CDR1, VL CDR2 and VL CDR3 of the second binding domain form a binding site for an epitope of the CD3.

66. The isolated multispecific antibody of claim 40, wherein the PSMA is present on the surface of a cell.

67. The isolated multispecific antibody of claim 66, wherein the cell is a prostate cell.

68. The isolated multispecific antibody of claim 66, wherein the cell is a prostate cancer cell.

69. The isolated multispecific antibody of claim 66, wherein the cell is a renal cell.

70. The isolated multispecific antibody of claim 66, wherein the cell is a renal cancer cell.

71. The isolated multispecific antibody of claim 66, wherein the renal cancer is a metastatic renal cell carcinoma.

72. The isolated multispecific antibody of claim 40, wherein the antibody is a bispecific antibody.

73. The isolated multispecific antibody of claim 40, wherein the antibody is a trispecific antibody.

74. The isolated multispecific antibody of claim 40, wherein the antibody is a quadraspecific antibody.

75. A nucleic acid encoding the multispecific antibody of claim 40.

76. A vector comprising the nucleic acid of claim 75.

77. A host cell comprising the vector of claim 76.

78. A kit comprising the vector of claim 76 and packaging for the same.

79. A kit comprising the multispecific antibody of claim 40 and packaging for the same.

80. A pharmaceutical composition comprising the multispecific antibody of claim 40, and a pharmaceutically acceptable carrier.

81. A method of producing the pharmaceutical composition of claim 80, comprising combining the multispecific antibody with a pharmaceutically acceptable carrier to obtain the pharmaceutical composition.

82. A method of directing a CD3-expressing T cell to a PSMA-expressing target cell, comprising contacting the T cell with the multispecific antibody of claim 40.

83. The method of claim 82, wherein the contacting directs the T cell to the target cell.

84. A method of inhibiting the growth or proliferation of a PSMA-expressing target cell, comprising contacting the target cell with the multispecific antibody of claim 40.

85. The method of claim 84, wherein the contacting is in the presence of CD3-expressing T cells.

86. A method of eliminating a PSMA-expressing target cell in a subject, comprising administering to the subject an effective amount of the multispecific antibody of claim 40.

87. The method of claim 86, wherein the subject has a disease or disorder of the prostate.

88. The method of claim 86, wherein the subject has prostate inflammation.

89. The method of claim 86, wherein the subject has Benign prostatic hyperplasia.

90. The method of claim 86, wherein the subject has a prostate cancer.

91. The method of claim 90, wherein the subject has metastatic castration-resistant prostate cancer (mCRPC).

92. The method of claim 86, wherein the target cell expresses PSMA on the cell surface.

93. The method of claim 86, wherein the target cell is a prostate cell.

94. The method of claim 86, wherein the target cell is a prostate cancer cell.

95. The method of claim 86, wherein the subject has a renal disease or disorder.

96. The method of claim 86, wherein the subject has a renal cancer.

97. The method of claim 86, wherein the subject has a metastatic renal cell carcinoma.

98. The method of claim 86, wherein the target cell expresses PSMA on the cell surface.

99. The method of claim 86, wherein the target cell is a renal cell.

100. The method of claim 86, wherein the target cell is a renal cancer cell.

101. The method of claim 86, wherein the target cell is a metastatic renal cell carcinoma cell.

102. The method of claim 86, wherein the subject is a subject in need thereof.

103. A method of treating a disease or disorder in a subject, comprising administering to the subject an effective amount of the multispecific antibody of claim 40.

104. The method of claim 103, wherein the disease or disorder is a disease or disorder of the prostate.

105. The method of claim 103, wherein the disease or disorder of the prostate is prostate inflammation.

106. The method of claim 103, wherein the disease or disorder of the prostate is Benign prostatic hyperplasia.

107. The method of claim 103, wherein the disease or disorder of the prostate is a prostate cancer.

108. The method of claim 107, wherein the disease or disorder is metastatic castration-resistant prostate cancer (mCRPC).

109. The method of claim 103, wherein the disease or disorder is a renal disease or disorder.

110. The method of claim 103, wherein the renal disease or disorder is a renal cancer.

111. The method of claim 103, wherein the renal disease or disorder is a metastatic renal cell carcinoma.

112. The method of claim 103, wherein the subject is a subject in need thereof.


SEQUENCE LISTING (1534 sequences)

SEQ ID NO: 1 - protein, 5 aa, Artificial Sequence
  Description of Artificial Sequence: Synthetic peptide
RYNMN

SEQ ID NO: 2 - protein, 17 aa, Artificial Sequence
  Description of Artificial Sequence: Synthetic peptide
SINSNSRYIYYADSVKG

SEQ ID NO: 3 - protein, 13 aa, Artificial Sequence
  Description of Artificial Sequence: Synthetic peptide
TMGDYYYYYGMDV

SEQ ID NO: 4 - protein, 6 aa, Artificial Sequence
  Description of Artificial Sequence: Synthetic peptide
NSNSRY

SEQ ID NO: 5 - protein, 7 aa, Artificial Sequence
  Description of Artificial Sequence: Synthetic peptide
DNSNRPS

SEQ ID NO: 6 - protein, 11 aa, Artificial Sequence
  Description of Artificial Sequence: Synthetic peptide
QSYDSSLSGVV

SEQ ID NO: 7 - protein, 7 aa, Artificial Sequence
  Description of Artificial Sequence: Synthetic peptide
GFTFSRY

SEQ ID NO: 8 - skipped

SEQ ID NO: 9 - protein, 11 aa, Artificial Sequence
  Description of Artificial Sequence: Synthetic peptide
MGDYYYYYGMD

SEQ ID NO: 10 - protein, 10 aa, Artificial Sequence
  Description of Artificial Sequence: Synthetic peptide
SSFNLGAGYD

SEQ ID NO: 11 - protein, 3 aa, Artificial Sequence
  Description of Artificial Sequence: Synthetic peptide
DNS

SEQ ID NO: 12 - protein, 8 aa, Artificial Sequence
  Description of Artificial Sequence: Synthetic peptide
YDSSLSGV

SEQ ID NO: 13 - protein, 10 aa, Artificial Sequence
  Description of Artificial Sequence: Synthetic peptide
GFTFSRYNMN

SEQ ID NO: 14 - protein, 10 aa, Artificial Sequence
  Description of Artificial Sequence: Synthetic peptide
SINSNSRYIY

SEQ ID NO: 15 - skipped

SEQ ID NO: 16 - protein, 14 aa, Artificial Sequence
  Description of Artificial Sequence: Synthetic peptide
TGSSFNLGAGYDVH

SEQ ID NO: 17-18 - skipped (2 entries)

SEQ ID NO: 19 - protein, 6 aa, Artificial Sequence
  Description of Artificial Sequence: Synthetic peptide
SRYNMN

SEQ ID NO: 20 - protein, 13 aa, Artificial Sequence
  Description of Artificial Sequence: Synthetic peptide
WVSSINSNSRYIY

SEQ ID NO: 21 - protein, 14 aa, Artificial Sequence
  Description of Artificial Sequence: Synthetic peptide
AKTMGDYYYYYGMD

SEQ ID NO: 22 - protein, 10 aa, Artificial Sequence
  Description of Artificial Sequence: Synthetic peptide
LGAGYDVHWY

SEQ ID NO: 23 - protein, 10 aa, Artificial Sequence
  Description of Artificial Sequence: Synthetic peptide
LLIYDNSNRP

SEQ ID NO: 24 - protein, 10 aa, Artificial Sequence
  Description of Artificial Sequence: Synthetic peptide
QSYDSSLSGV

SEQ ID NO: 25 - protein, 8 aa, Artificial Sequence
  Description of Artificial Sequence: Synthetic peptide
GFTFSRYN

SEQ ID NO: 26 - protein, 8 aa, Artificial Sequence
  Description of Artificial Sequence: Synthetic peptide
INSNSRYI

SEQ ID NO: 27 - protein, 15 aa, Artificial Sequence
  Description of Artificial Sequence: Synthetic peptide
AKTMGDYYYYYGMDV

SEQ ID NO: 28 - protein, 9 aa, Artificial Sequence
  Description of Artificial Sequence: Synthetic peptide
SFNLGAGYD

SEQ ID NO: 29-30 - skipped (2 entries)

SEQ ID NO: 31 - protein, 122 aa, Artificial Sequence
  Description of Artificial Sequence: Synthetic polypeptide
EVQLVESGGGLVKPGGSLRLSCAASGFTFSRYNMNWVRQAPGKGLEWVSSINSNSRYIYYADSVKGRFTISRDSAKNSLYLQMNSLRAEDTAVYYCAKTMGDYYYYYGMDVWGQGTTVTVSS

SEQ ID NO: 32 - protein, 111 aa, Artificial Sequence
  Description of Artificial Sequence: Synthetic polypeptide
QSVLTQPPSVSGAPGQRVTISCTGSSFNLGAGYDVHWYQQVPGTVPKLLIYDNSNRPSGVPDRFSGSKSGTSASLAITGLQAEDETVYYCQSYDSSLSGVVFGGGTKLTVL

SEQ ID NO: 33 - protein, 451 aa, Artificial Sequence
  Description of Artificial Sequence: Synthetic polypeptide
EVQLVESGGGLVKPGGSLRLSCAASGFTFSRYNMNWVRQAPGKGLEWVSSINSNSRYIYYADSVKGRFTISRDSAKNSLYLQMNSLRAEDTAVYYCAKTMGDYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 34 - protein, 217 aa, Artificial Sequence
  Description of Artificial Sequence: Synthetic polypeptide
QSVLTQPPSVSGAPGQRVTISCTGSSFNLGAGYDVHWYQQVPGTVPKLLIYDNSNRPSGVPDRFSGSKSGTSASLAITGLQAEDETVYYCQSYDSSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35-37 - skipped (3 entries)

SEQ ID NO: 38 - protein, 14 aa, Artificial Sequence
  Description of Artificial Sequence: Synthetic peptide
AGSLSNIGAGYDVH

SEQ ID NO: 39 - protein, 7 aa, Artificial Sequence
  Description of Artificial Sequence: Synthetic peptide
GNINRLS

SEQ ID NO: 40 - protein, 11 aa, Artificial Sequence
  Description of Artificial Sequence: Synthetic peptide
QSYDSSLSSYV

SEQ ID NO: 41-43 - skipped (3 entries)

SEQ ID NO: 44 - protein, 10 aa, Artificial Sequence
  Description of Artificial Sequence: Synthetic peptide
SLSNIGAGYD

SEQ ID NO: 45 - protein, 3 aa, Artificial Sequence
  Description of Artificial Sequence: Synthetic peptide
GNI

SEQ ID NO: 46 - protein, 8 aa, Artificial Sequence
  Description of Artificial Sequence: Synthetic peptide
YDSSLSSY

SEQ ID NO: 47-55 - skipped (9 entries)

SEQ ID NO: 56 - protein, 10 aa, Artificial Sequence
  Description of Artificial Sequence: Synthetic peptide
IGAGYDVHWY

SEQ ID NO: 57 - protein, 10 aa, Artificial Sequence
  Description of Artificial Sequence: Synthetic peptide
LLIYGNINRL

SEQ ID NO: 58 - protein, 10 aa, Artificial Sequence
  Description of Artificial Sequence: Synthetic peptide
QSYDSSLSSY

SEQ ID NO: 59-61 - skipped (3 entries)

SEQ ID NO: 62 - protein, 9 aa, Artificial Sequence
  Description of Artificial Sequence: Synthetic peptide
LSNIGAGYD

SEQ ID NO: 63-65 - skipped (3 entries)

SEQ ID NO: 66 - protein, 111 aa, Artificial Sequence
  Description of Artificial Sequence: Synthetic polypeptide
SSELTQPPSVSGAPGQRVTISCAGSLSNIGAGYDVHWYQQLPGTAPKLLIYGNINRLSGVPERFSGSKSGTSASLAITGLQAEDGADYYCQSYDSSLSSYVFGTGTKVTVL

SEQ ID NO: 67 - skipped

SEQ ID NO: 68 - protein, 217 aa, Artificial Sequence
  Description of Artificial Sequence: Synthetic polypeptide
SSELTQPPSVSGAPGQRVTISCAGSLSNIGAGYDVHWYQQLPGTAPKLLIYGNINRLSGVPERFSGSKSGTSASLAITGLQAEDGADYYCQSYDSSLSSYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 69 - protein, 5 aa, Artificial Sequence
  Description of Artificial Sequence: Synthetic peptide
TYGMH

SEQ ID NO: 70 - protein, 17 aa, Artificial Sequence
  Description of Artificial Sequence: Synthetic peptide
VVSFDESNKYYADSVKG

SEQ ID NO: 71 - protein, 16 aa, Artificial Sequence
  Description of Artificial Sequence: Synthetic peptide
ALRDGNNWDYFNGMDV

SEQ ID NO: 72 - protein, 13 aa, Artificial Sequence
  Description of Artificial Sequence: Synthetic peptide
SGSSSNIGSNTVN

SEQ ID NO: 73 - protein, 7 aa, Artificial Sequence
  Description of Artificial Sequence: Synthetic peptide
SDNQRPS

SEQ ID NO: 74 - protein, 11 aa, Artificial Sequence
  Description of Artificial Sequence: Synthetic peptide
AAWDDSLNGYV

SEQ ID NO: 75 - protein, 7 aa, Artificial Sequence
  Description of Artificial Sequence: Synthetic peptide
GFTFITY

SEQ ID NO: 76 - protein, 6 aa, Artificial Sequence
  Description of Artificial Sequence: Synthetic peptide
SFDESN

SEQ ID NO: 77 - protein, 14 aa, Artificial Sequence
  Description of Artificial Sequence: Synthetic peptide
LRDGNNWDYFNGMD

SEQ ID NO: 78 - protein, 9 aa, Artificial Sequence
  Description of Artificial Sequence: Synthetic peptide
SSSNIGSNT

SEQ ID NO: 79 - protein, 3 aa, Artificial Sequence
  Description of Artificial Sequence: Synthetic peptide
SDN

SEQ ID NO: 80 - protein, 8 aa, Artificial Sequence
  Description of Artificial Sequence: Synthetic peptide
WDDSLNGY

SEQ ID NO: 81 - protein, 10 aa, Artificial Sequence
  Description of Artificial Sequence: Synthetic peptide
GFTFITYGMH

SEQ ID NO: 82 - protein, 10 aa, Artificial Sequence
  Description of Artificial Sequence: Synthetic peptide
VVSFDESNKY

SEQ ID NO: 83-86 - skipped (4 entries)

SEQ ID NO: 87 - protein, 6 aa, Artificial Sequence
  Description of Artificial Sequence: Synthetic peptide
ITYGMH

SEQ ID NO: 88 - protein, 13 aa, Artificial Sequence
  Description of Artificial Sequence: Synthetic peptide
WVAVVSFDESNKY

SEQ ID NO: 89 - protein, 17 aa, Artificial Sequence
  Description of Artificial Sequence: Synthetic peptide
ARALRDGNNWDYFNGMD

SEQ ID NO: 90 - protein, 9 aa, Artificial Sequence
  Description of Artificial Sequence: Synthetic peptide
IGSNTVNWF

SEQ ID NO: 91 - protein, 10 aa, Artificial Sequence
  Description of Artificial Sequence: Synthetic peptide
LLIYSDNQRP

SEQ ID NO: 92 - protein, 10 aa, Artificial Sequence
  Description of Artificial Sequence: Synthetic peptide
AAWDDSLNGY

SEQ ID NO: 93 - protein, 8 aa, Artificial Sequence
  Description of Artificial Sequence: Synthetic peptide
GFTFITYG

SEQ ID NO: 94 - protein, 8 aa, Artificial Sequence
  Description of Artificial Sequence: Synthetic peptide
VSFDESNK

SEQ ID NO: 95 - protein, 18 aa, Artificial Sequence
  Description of Artificial Sequence: Synthetic peptide
ARALRDGNNWDYFNGMDV

SEQ ID NO: 96 - protein, 8 aa, Artificial Sequence
  Description of Artificial Sequence: Synthetic peptide
SSNIGSNT

SEQ ID NO: 97-98 - skipped (2 entries)

SEQ ID NO: 99 - protein, 125 aa, Artificial Sequence
  Description of Artificial Sequence: Synthetic polypeptide
EVQLVESGGGVVQPGRSLRLSCAASGFTFITYGMHWVRQAPGKGLEWVAVVSFDESNKYYADSVKGRFTISRDNSKNTLYLQMNSLRAEDTAVYYCARALRDGNNWDYFNGMDVWGQGTTVTVSS

SEQ ID NO: 100 - protein, 110 aa, Artificial Sequence
  Description of Artificial Sequence: Synthetic polypeptide
QSVLTQPPSASGTPGQGVTISCSGSSSNIGSNTVNWFQQLPGTAPKLLIYSDNQRPSGVPDRFSGSKSGTSASLAISGLQSEDEADYYCAAWDDSLNGYVFGTGTKVTVL

SEQ ID NO: 101 - protein, 454 aa, Artificial Sequence
  Description of Artificial Sequence: Synthetic polypeptide
EVQLVESGGGVVQPGRSLRLSCAASGFTFITYGMHWVRQAPGKGLEWVAVVSFDESNKYYADSVKGRFTISRDNSKNTLYLQMNSLRAEDTAVYYCARALRDGNNWDYFNGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 102 - protein, 216 aa, Artificial Sequence
  Description of Artificial Sequence: Synthetic polypeptide
QSVLTQPPSASGTPGQGVTISCSGSSSNIGSNTVNWFQQLPGTAPKLLIYSDNQRPSGVPDRFSGSKSGTSASLAISGLQSEDEADYYCAAWDDSLNGYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103-105 - skipped (3 entries)

SEQ ID NO: 106 - protein, 14 aa, Artificial Sequence
  Description of Artificial Sequence: Synthetic peptide
TGSSSNIGADYDVH

SEQ ID NO: 107 - protein, 7 aa, Artificial Sequence
  Description of Artificial Sequence: Synthetic peptide
GNSNRPS

SEQ ID NO: 108 - protein, 11 aa, Artificial Sequence
  Description of Artificial Sequence: Synthetic peptide
QSYDSSLSGWV

SEQ ID NO: 109-111 - skipped (3 entries)

SEQ ID NO: 112 - protein, 10 aa, Artificial Sequence
  Description of Artificial Sequence: Synthetic peptide
SSSNIGADYD

SEQ ID NO: 113 - protein, 3 aa, Artificial Sequence
  Description of Artificial Sequence: Synthetic peptide
GNS

SEQ ID NO: 114 - protein, 8 aa, Artificial Sequence
  Description of Artificial Sequence: Synthetic peptide
YDSSLSGW

SEQ ID NO: 115-123 - skipped (9 entries)

SEQ ID NO: 124 - protein, 10 aa, Artificial Sequence
  Description of Artificial Sequence: Synthetic peptide
IGADYDVHWY

SEQ ID NO: 125 - protein, 10 aa, Artificial Sequence
  Description of Artificial Sequence: Synthetic peptide
LLIYGNSNRP

SEQ ID NO: 126 - protein, 10 aa, Artificial Sequence
  Description of Artificial Sequence: Synthetic peptide
QSYDSSLSGW

SEQ ID NO: 127-129 - skipped (3 entries)

SEQ ID NO: 130 - protein, 9 aa, Artificial Sequence
  Description of Artificial Sequence: Synthetic peptide
SSNIGADYD

SEQ ID NO: 131-133 - skipped (3 entries)

SEQ ID NO: 134 - protein, 111 aa, Artificial Sequence
  Description of Artificial Sequence: Synthetic polypeptide
QSVLTQPPSVSGAPGQRVTISCTGSSSNIGADYDVHWYQHLPGTAPKLLIYGNSNRPSGVPDRFSGSKSGTSASLAITGLQAEDETDYYCQSYDSSLSGWVFGGGTKLTVL

SEQ ID NO: 135 - skipped

SEQ ID NO: 136 - protein, 217 aa, Artificial Sequence
  Description of Artificial Sequence: Synthetic polypeptide
QSVLTQPPSVSGAPGQRVTISCTGSSSNIGADYDVHWYQHLPGTAPKLLIYGNSNRPSGVPDRFSGSKSGTSASLAITGLQAEDETDYYCQSYDSSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 137 - protein, 5 aa, Artificial Sequence
  Description of Artificial Sequence: Synthetic peptide
SYGIH

SEQ ID NO: 138 - protein, 17 aa, Artificial Sequence
  Description of Artificial Sequence: Synthetic peptide
VIWYDGSNKYYADSVKG

SEQ ID NO: 139 - protein, 15 aa, Artificial Sequence
  Description of Artificial Sequence: Synthetic peptide
GVGPTSYYYNYGMDV

SEQ ID NO: 140-142 - skipped (3 entries)

SEQ ID NO: 143 - protein, 7 aa, Artificial Sequence
  Description of Artificial Sequence: Synthetic peptide
GFTFSSY

SEQ ID NO: 144 - protein, 6 aa, Artificial Sequence
  Description of Artificial Sequence: Synthetic peptide
WYDGSN

SEQ ID NO: 145 - protein, 13 aa, Artificial Sequence
  Description of Artificial Sequence: Synthetic peptide
VGPTSYYYNYGMD

SEQ ID NO: 146-148 - skipped (3 entries)

SEQ ID NO: 149 - protein, 10 aa, Artificial Sequence
  Description of Artificial Sequence: Synthetic peptide
GFTFSSYGIH

SEQ ID NO: 150 - protein, 10 aa, Artificial Sequence
  Description of Artificial Sequence: Synthetic peptide
VIWYDGSNKY

SEQ ID NO: 151-154 - skipped (4 entries)

SEQ ID NO: 155 - protein, 6 aa, Artificial Sequence
  Description of Artificial Sequence: Synthetic peptide
SSYGIH

SEQ ID NO: 156 - protein, 13 aa, Artificial Sequence
  Description of Artificial Sequence: Synthetic peptide
WVAVIWYDGSNKY

SEQ ID NO: 157 - protein, 16 aa, Artificial Sequence
  Description of Artificial Sequence: Synthetic peptide
VRGVGPTSYYYNYGMD

SEQ ID NO: 158-160 - skipped (3 entries)

SEQ ID NO: 161 - protein, 8 aa, Artificial Sequence
  Description of Artificial Sequence: Synthetic peptide
GFTFSSYG

SEQ ID NO: 162 - protein, 8 aa, Artificial Sequence
  Description of Artificial Sequence: Synthetic peptide
IWYDGSNK

SEQ ID NO: 163 - protein, 17 aa, Artificial Sequence
  Description of Artificial Sequence: Synthetic peptide
VRGVGPTSYYYNYGMDV

SEQ ID NO: 164-166 - skipped (3 entries)

SEQ ID NO: 167 - protein, 124 aa, Artificial Sequence
  Description of Artificial Sequence: Synthetic polypeptide
QVQLVESGGGVVQPGRSLRLSCVASGFTFSSYGIHWVRQAPGKGLEWVAVIWYDGSNKYYADSVKGRFTISRDNSKNTLYLQMNSLRAEDTAVYYSVRGVGPTSYYYNYGMDVWGQGTTVTVSS

SEQ ID NO: 168 - skipped

SEQ ID NO: 169 - protein, 453 aa, Artificial Sequence
  Description of Artificial Sequence: Synthetic polypeptide
QVQLVESGGGVVQPGRSLRLSCVASGFTFSSYGIHWVRQAPGKGLEWVAVIWYDGSNKYYADSVKGRFTISRDNSKNTLYLQMNSLRAEDTAVYYSVRGVGPTSYYYNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 170-204 - skipped (35 entries)

SEQ ID NO: 205 - protein, 5 aa, Artificial Sequence
  Description of Artificial Sequence: Synthetic peptide
SYAMS

SEQ ID NO: 206 - protein, 17 aa, Artificial Sequence
  Description of Artificial Sequence: Synthetic peptide
AISGGIGSTYYADSVKG

SEQ ID NO: 207 - protein, 12 aa, Artificial Sequence
  Description of Artificial Sequence: Synthetic peptide
DGVGATPYYFDY

SEQ ID NO: 208 - protein, 13 aa, Artificial Sequence
  Description of Artificial Sequence: Synthetic peptide
SGSSSNIGNNYVS

SEQ ID NO: 209 - protein, 7 aa, Artificial Sequence
  Description of Artificial Sequence: Synthetic peptide
DNNKRPS

SEQ ID NO: 210 - protein, 11 aa, Artificial Sequence
  Description of Artificial Sequence: Synthetic peptide
GTWDSSLSAYV

SEQ ID NO: 211 - skipped

SEQ ID NO: 212 - protein, 6 aa, Artificial Sequence
  Description of Artificial Sequence: Synthetic peptide
SGGIGS

SEQ ID NO: 213 - protein, 10 aa, Artificial Sequence
  Description of Artificial Sequence: Synthetic peptide
GVGATPYYFD

SEQ ID NO: 214 - protein, 9 aa, Artificial Sequence
  Description of Artificial Sequence: Synthetic peptide
SSSNIGNNY

SEQ ID NO: 215 - protein, 3 aa, Artificial Sequence
  Description of Artificial Sequence: Synthetic peptide
DNN

SEQ ID NO: 216 - protein, 8 aa, Artificial Sequence
  Description of Artificial Sequence: Synthetic peptide
WDSSLSAY

SEQ ID NO: 217 - protein, 10 aa, Artificial Sequence
  Description of Artificial Sequence: Synthetic peptide
GFTFSSYAMS

SEQ ID NO: 218 - protein, 10 aa, Artificial Sequence
  Description of Artificial Sequence: Synthetic peptide
AISGGIGSTY

SEQ ID NO: 219-222 - skipped (4 entries)

SEQ ID NO: 223 - protein, 6 aa, Artificial Sequence
  Description of Artificial Sequence: Synthetic peptide
SSYAMS

SEQ ID NO: 224 - protein, 13 aa, Artificial Sequence
  Description of Artificial Sequence: Synthetic peptide
WVSAISGGIGSTY

SEQ ID NO: 225 - protein, 13 aa, Artificial Sequence
  Description of Artificial Sequence: Synthetic peptide
AKDGVGATPYYFD

SEQ ID NO: 226 - protein, 9 aa, Artificial Sequence
  Description of Artificial Sequence: Synthetic peptide
IGNNYVSWY

SEQ ID NO: 227 - protein, 10 aa, Artificial Sequence
  Description of Artificial Sequence: Synthetic peptide
LLIYDNNKRP

SEQ ID NO: 228 - protein, 10 aa, Artificial Sequence
  Description of Artificial Sequence: Synthetic peptide
GTWDSSLSAY

SEQ ID NO: 229 - protein, 8 aa, Artificial Sequence
  Description of Artificial Sequence: Synthetic peptide
GFTFSSYA

SEQ ID NO: 230 - protein, 8 aa, Artificial Sequence
  Description of Artificial Sequence: Synthetic peptide
ISGGIGST

SEQ ID NO: 231 - protein, 14 aa, Artificial Sequence
  Description of Artificial Sequence: Synthetic peptide
AKDGVGATPYYFDY

SEQ ID NO: 232 - protein, 8 aa, Artificial Sequence
  Description of Artificial Sequence: Synthetic peptide
SSNIGNNY

SEQ ID NO: 233-234 - skipped (2 entries)

SEQ ID NO: 235 - protein, 121 aa, Artificial Sequence
  Description of Artificial Sequence: Synthetic polypeptide
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSS

SEQ ID NO: 236 - protein, 110 aa, Artificial Sequence
  Description of Artificial Sequence: Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVL

SEQ ID NO: 237 - protein, 450 aa, Artificial Sequence
  Description of Artificial Sequence: Synthetic polypeptide
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 238 - protein, 216 aa, Artificial Sequence
  Description of Artificial Sequence: Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 239-241 - skipped (3 entries)

SEQ ID NO: 242 - protein, 13 aa, Artificial Sequence
  Description of Artificial Sequence: Synthetic peptide
SGSSSNIGINYVS

SEQ ID NO: 243 - skipped

SEQ ID NO: 244 - protein, 11 aa, Artificial Sequence
  Description of Artificial Sequence: Synthetic peptide
GTWDSSLSAVV

SEQ ID NO: 245-247 - skipped (3 entries)

SEQ ID NO: 248 - protein, 9 aa, Artificial Sequence
  Description of Artificial Sequence: Synthetic peptide
SSSNIGINY

SEQ ID NO: 249 - skipped

SEQ ID NO: 250 - protein, 8 aa, Artificial Sequence
  Description of Artificial Sequence: Synthetic peptide
WDSSLSAV

SEQ ID NO: 251-259 - skipped (9 entries)

SEQ ID NO: 260 - protein, 9 aa, Artificial Sequence
  Description of Artificial Sequence: Synthetic peptide
IGINYVSWY

SEQ ID NO: 261 - skipped

SEQ ID NO: 262 - protein, 10 aa, Artificial Sequence
  Description of Artificial Sequence: Synthetic peptide
GTWDSSLSAV

SEQ ID NO: 263-265 - skipped (3 entries)

SEQ ID NO: 266 - protein, 8 aa, Artificial Sequence
  Description of Artificial Sequence: Synthetic peptide
SSNIGINY

SEQ ID NO: 267-269 - skipped (3 entries)

SEQ ID NO: 270 - protein, 110 aa, Artificial Sequence
  Description of Artificial Sequence: Synthetic polypeptide
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVL

SEQ ID NO: 271 - skipped

SEQ ID NO: 272 - protein, 216 aa, Artificial Sequence
  Description of Artificial Sequence: Synthetic polypeptide
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 273 - skipped

SEQ ID NO: 274 - protein, 17 aa, Artificial Sequence
  Description of Artificial Sequence: Synthetic peptide
AISGGSGSTYYADSVKG

SEQ ID NO: 275-279 - skipped (5 entries)

SEQ ID NO: 280 - protein, 6 aa, Artificial Sequence
  Description of Artificial Sequence: Synthetic peptide
SGGSGS

SEQ ID NO: 281-285 - skipped (5 entries)

SEQ ID NO: 286 - protein, 10 aa, Artificial Sequence
  Description of Artificial Sequence: Synthetic peptide
AISGGSGSTY

SEQ ID NO: 287-291 - skipped (5 entries)

SEQ ID NO: 292 - protein, 13 aa, Artificial Sequence
  Description of Artificial Sequence: Synthetic peptide
WVSAISGGSGSTY

SEQ ID NO: 293-297 - skipped (5 entries)

SEQ ID NO: 298 - protein, 8 aa, Artificial Sequence
  Description of Artificial Sequence: Synthetic peptide
ISGGSGST

SEQ ID NO: 299-302 - skipped (4 entries)

SEQ ID NO: 303 - protein, 121 aa, Artificial Sequence
  Description of Artificial Sequence: Synthetic polypeptide
EVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSS

SEQ ID NO: 304 - skipped

SEQ ID NO: 305 - protein, 450 aa, Artificial Sequence
  Description of Artificial Sequence: Synthetic polypeptide
EVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 306-341 - skipped (36 entries)

SEQ ID NO: 342 - protein, 17 aa, Artificial Sequence
  Description of Artificial Sequence: Synthetic peptide
AISGGYGAPYYADTVKG

SEQ ID NO: 343 - protein, 12 aa, Artificial Sequence
  Description of Artificial Sequence: Synthetic peptide
DGVGATPYYFDD

SEQ ID NO: 344-346 - skipped (3 entries)

SEQ ID NO: 347 - protein, 7 aa, Artificial Sequence
  Description of Artificial Sequence: Synthetic peptide
GFIFSSY

SEQ ID NO: 348 - protein, 6 aa, Artificial Sequence
  Description of Artificial Sequence: Synthetic peptide
SGGYGA

SEQ ID NO: 349-352 - skipped (4 entries)

SEQ ID NO: 353 - protein, 10 aa, Artificial Sequence
  Description of Artificial Sequence: Synthetic peptide
GFIFSSYAMS

SEQ ID NO: 354 - protein, 10 aa, Artificial Sequence
  Description of Artificial Sequence: Synthetic peptide
AISGGYGAPY

SEQ ID NO: 355-359 - skipped (5 entries)

SEQ ID NO: 360 - protein, 13 aa, Artificial Sequence
  Description of Artificial Sequence: Synthetic peptide
WVSAISGGYGAPY

SEQ ID NO: 361-362 - skipped (2 entries)

SEQ ID NO: 363 - protein, 10 aa, Artificial Sequence
  Description of Artificial Sequence: Synthetic peptide
LLIFDNNKRP

SEQ ID NO: 364 - skipped

SEQ ID NO: 365 - protein, 8 aa, Artificial Sequence
  Description of Artificial Sequence: Synthetic peptide
GFIFSSYA

SEQ ID NO: 366 - protein, 8 aa, Artificial Sequence
  Description of Artificial Sequence: Synthetic peptide
ISGGYGAP

SEQ ID NO: 367 - protein, 14 aa, Artificial Sequence
  Description of Artificial Sequence: Synthetic peptide
AKDGVGATPYYFDD

SEQ ID NO: 368-370 - skipped (3 entries)

SEQ ID NO: 371 - protein, 121 aa, Artificial Sequence
  Description of Artificial Sequence: Synthetic polypeptide
EVQLVESGGGLVQPGGSLRLSCTASGFIFSSYAMSWVRQAPGKGLEWVSAISGGYGAPYYADTVKGRFTISRDNSKNTLYLQMNSLRAEDTAVYYCAKDGVGATPYYFDDWGQGILVTVSS

SEQ ID NO: 372 - protein, 110 aa, Artificial Sequence
  Description of Artificial Sequence: Synthetic polypeptide
QSVLTQPPSVSAAPGQKVTISCSGSSSNIGNNYVSWYQQLPGTAPKLLIFDNNKRPSGIPDRFSGSKSGTSATLGITGLQTGDEADYYCGTWDSSLSAYVFGTGTKVTVL

SEQ ID NO: 373 - protein, 450 aa, Artificial Sequence
  Description of Artificial Sequence: Synthetic polypeptide
EVQLVESGGGLVQPGGSLRLSCTASGFIFSSYAMSWVRQAPGKGLEWVSAISGGYGAPYYADTVKGRFTISRDNSKNTLYLQMNSLRAEDTAVYYCAKDGVGATPYYFDDWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 374 - protein, 216 aa, Artificial Sequence
  Description of Artificial Sequence: Synthetic polypeptide
QSVLTQPPSVSAAPGQKVTISCSGSSSNIGNNYVSWYQQLPGTAPKLLIFDNNKRPSGIPDRFSGSKSGTSATLGITGLQTGDEADYYCGTWDSSLSAYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 375 - protein, 5 aa, Artificial Sequence
  Description of Artificial Sequence: Synthetic peptide
FYSMN

SEQ ID NO: 376 - protein, 17 aa, Artificial Sequence
  Description of Artificial Sequence: Synthetic peptide
SISSSGNYIYYADSVKG

SEQ ID NO: 377 - protein, 11 aa, Artificial Sequence
  Description of Artificial Sequence: Synthetic peptide
SYSGSYDAFDF

SEQ ID NO: 378 - protein, 12 aa, Artificial Sequence
  Description of Artificial Sequence: Synthetic peptide
RASQSVSSSFLA

SEQ ID NO: 379 - protein, 7 aa, Artificial Sequence
  Description of Artificial Sequence: Synthetic peptide
GASSRAT

SEQ ID NO: 380 - protein, 9 aa, Artificial Sequence
  Description of Artificial Sequence: Synthetic peptide
QQYGVSPWT

SEQ ID NO: 381 - protein, 7 aa, Artificial Sequence
  Description of Artificial Sequence: Synthetic peptide
GFTFSFY

SEQ ID NO: 382 - protein, 6 aa, Artificial Sequence
  Description of Artificial Sequence: Synthetic peptide
SSSGNY

SEQ ID NO: 383 - protein, 10 aa, Artificial Sequence
  Description of Artificial Sequence: Synthetic peptide
SYSGSYDAFD

SEQ ID NO: 384 - protein, 8 aa, Artificial Sequence
  Description of Artificial Sequence: Synthetic peptide
SQSVSSSF

SEQ ID NO: 385 - protein, 3 aa, Artificial Sequence
  Description of Artificial Sequence: Synthetic peptide
GAS

SEQ ID NO: 386 - protein, 6 aa, Artificial Sequence
  Description of Artificial Sequence: Synthetic peptide
YGVSPW

SEQ ID NO: 387 - protein, 10 aa, Artificial Sequence
  Description of Artificial Sequence: Synthetic peptide
GFTFSFYSMN

SEQ ID NO: 388 - protein, 10 aa, Artificial Sequence
  Description of Artificial Sequence: Synthetic peptide
SISSSGNYIY

SEQ ID NO: 389-392 - skipped (4 entries)

SEQ ID NO: 393 - protein, 6 aa, Artificial Sequence
  Description of Artificial Sequence: Synthetic peptide
SFYSMN

SEQ ID NO: 394 - protein, 13 aa, Artificial Sequence
  Description of Artificial Sequence: Synthetic peptide
WVSSISSSGNYIY

SEQ ID NO: 395 - protein, 12 aa, Artificial Sequence
  Description of Artificial Sequence: Synthetic peptide
ARSYSGSYDAFD

SEQ ID NO: 396 - protein, 8 aa, Artificial Sequence
  Description of Artificial Sequence: Synthetic peptide
SSSFLAWY

SEQ ID NO: 397 - protein, 10 aa, Artificial Sequence
  Description of Artificial Sequence: Synthetic peptide
LLISGASSRA

SEQ ID NO: 398 - protein, 8 aa, Artificial Sequence
  Description of Artificial Sequence: Synthetic peptide
QQYGVSPW

SEQ ID NO: 399 - protein, 8 aa, Artificial Sequence
  Description of Artificial Sequence: Synthetic peptide
GFTFSFYS

SEQ ID NO: 400 - protein, 8 aa, Artificial Sequence
  Description of Artificial Sequence: Synthetic peptide
ISSSGNYI

SEQ ID NO: 401 - protein, 13 aa, Artificial Sequence
  Description of Artificial Sequence: Synthetic peptide
ARSYSGSYDAFDF

SEQ ID NO: 402 - protein, 7 aa, Artificial Sequence
  Description of Artificial Sequence: Synthetic peptide
QSVSSSF

SEQ ID NO: 403-404 - skipped (2 entries)

SEQ ID NO: 405 - protein, 120 aa, Artificial Sequence
  Description of Artificial Sequence: Synthetic polypeptide
EVQLVESGGGLVKPGGSLRLSCVASGFTFSFYSMNWVRQAPGKGLDWVSSISSSGNYIYYADSVKGRFTISRDNAKNSLHLHMNSLKAEDTAMYFCARSYSGSYDAFDFWGQGTMVTVSS

SEQ ID NO: 406 - protein, 108 aa, Artificial Sequence
  Description of Artificial Sequence: Synthetic polypeptide
EIVMTQSPGTLSLSPGERATLSCRASQSVSSSFLAWYQQKPGQAPRLLISGASSRATGIPDRFSVSGSGTDFTLTISRLEPEDFAVYYCQQYGVSPWTFGQGTKVEIK

SEQ ID NO: 407 - protein, 450 aa, Artificial Sequence
  Description of Artificial Sequence: Synthetic polypeptide
EVQLVESGGGLVKPGGSLRLSCVASGFTFSFYSMNWVRQAPGKGLDWVSSISSSGNYIYYADSVKGRFTISRDNAKNSLHLHMNSLKAEDTAMYFCARSYSGSYDAFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 408 - protein, 215 aa, Artificial Sequence
  Description of Artificial Sequence: Synthetic polypeptide
EIVMTQSPGTLSLSPGERATLSCRASQSVSSSFLAWYQQKPGQAPRLLISGASSRATGIPDRFSVSGSGTDFTLTISRLEPEDFAVYYCQQYGVSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 409-410 - skipped (2 entries)

SEQ ID NO: 411 - protein, 12 aa, Artificial Sequence
  Description of Artificial Sequence: Synthetic peptide
DAVGATPYYFDY

SEQ ID NO: 412-416 - skipped (5 entries)

SEQ ID NO: 417 - protein, 11 aa, Artificial Sequence
  Description of Artificial Sequence: Synthetic peptide
DAVGATPYYFD

SEQ ID NO: 418-428 - skipped (11 entries)

SEQ ID NO: 429 - protein, 13 aa, Artificial Sequence
  Description of Artificial Sequence: Synthetic peptide
AKDAVGATPYYFD

SEQ ID NO: 430-434 - skipped (5 entries)

SEQ ID NO: 435 - protein, 14 aa, Artificial Sequence
  Description of Artificial Sequence: Synthetic peptide
AKDAVGATPYYFDY

SEQ ID NO: 436-438 - skipped (3 entries)

SEQ ID NO: 439 - protein, 121 aa, Artificial Sequence
  Description of Artificial Sequence: Synthetic polypeptide
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSS

SEQ ID NO: 440 - skipped

SEQ ID NO: 441 - protein, 451 aa, Artificial Sequence
  Description of Artificial Sequence: Synthetic polypeptide
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 442 - skipped

SEQ ID NO: 443 - protein, 5 aa, Artificial Sequence
  Description of Artificial Sequence: Synthetic peptide
SYNMN

SEQ ID NO: 444 - protein, 17 aa, Artificial Sequence
  Description of Artificial Sequence: Synthetic peptide
IIYYDESNKYYADSVKG

SEQ ID NO: 445 - protein, 11 aa, Artificial Sequence
  Description of Artificial Sequence: Synthetic peptide
ERGRDYYGMDV

SEQ ID NO: 446 - protein, 11 aa, Artificial Sequence
  Description of Artificial Sequence: Synthetic peptide
SGDALPKQYAY

SEQ ID NO: 447 - protein, 7 aa, Artificial Sequence
  Description of Artificial Sequence: Synthetic peptide
KDSERPS

SEQ ID NO: 448 - protein, 10 aa, Artificial Sequence
  Description of Artificial Sequence: Synthetic peptide
QSADSSGTYV

SEQ ID NO: 449 - skipped

SEQ ID NO: 450 - protein, 6 aa, Artificial Sequence
  Description of Artificial Sequence: Synthetic peptide
YYDESN

SEQ ID NO: 451 - protein, 10 aa, Artificial Sequence
  Description of Artificial Sequence: Synthetic peptide
ERGRDYYGMD

SEQ ID NO: 452 - protein, 7 aa, Artificial Sequence
  Description of Artificial Sequence: Synthetic peptide
DALPKQY

SEQ ID NO: 453 - protein, 3 aa, Artificial Sequence
  Description of Artificial Sequence: Synthetic peptide
KDS

SEQ ID NO: 454 - protein, 7 aa, Artificial Sequence
  Description of Artificial Sequence: Synthetic peptide
ADSSGTY

SEQ ID NO: 455 - protein, 10 aa, Artificial Sequence
  Description of Artificial Sequence: Synthetic peptide
GFTFSSYNMN

SEQ ID NO: 456 - protein, 10 aa, Artificial Sequence
  Description of Artificial Sequence: Synthetic peptide
IIYYDESNKY

SEQ ID NO: 457-460 - skipped (4 entries)

SEQ ID NO: 461 - protein, 6 aa, Artificial Sequence
  Description of Artificial Sequence: Synthetic peptide
SSYNMN

SEQ ID NO: 462 - protein, 13 aa, Artificial Sequence
  Description of Artificial Sequence: Synthetic peptide
WVAIIYYDESNKY

SEQ ID NO: 463 - protein, 12 aa, Artificial Sequence
  Description of Artificial Sequence: Synthetic peptide
ARERGRDYYGMD

SEQ ID NO: 464 - protein, 7 aa, Artificial Sequence
  Description of Artificial Sequence: Synthetic peptide
KQYAYWY

SEQ ID NO: 465 - protein, 10 aa, Artificial Sequence
  Description of Artificial Sequence: Synthetic peptide
LVIYKDSERP

SEQ ID NO: 466 - protein, 9 aa, Artificial Sequence
  Description of Artificial Sequence: Synthetic peptide
QSADSSGTY

SEQ ID NO: 467 - protein, 8 aa, Artificial Sequence
  Description of Artificial Sequence: Synthetic peptide
GFTFSSYN

SEQ ID NO: 468 - protein, 8 aa, Artificial Sequence
  Description of Artificial Sequence: Synthetic peptide
IYYDESNK

SEQ ID NO: 469 - protein, 13 aa, Artificial Sequence
  Description of Artificial Sequence: Synthetic peptide
ARERGRDYYGMDV

SEQ ID NO: 470 - protein, 6 aa, Artificial Sequence
  Description of Artificial Sequence: Synthetic peptide
ALPKQY

SEQ ID NO: 471-472 - skipped (2 entries)

SEQ ID NO: 473 - protein, 120 aa, Artificial Sequence
  Description of Artificial Sequence: Synthetic polypeptide
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSS

SEQ ID NO: 474 - protein, 107 aa, Artificial Sequence
  Description of Artificial Sequence: Synthetic polypeptide
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVL

SEQ ID NO: 475-476 - skipped (2 entries)

SEQ ID NO: 477 - protein, 5 aa, Artificial Sequence
  Description of Artificial Sequence: Synthetic peptide
NYNMN

SEQ ID NO: 478 - protein, 17 aa, Artificial Sequence
  Description of Artificial Sequence: Synthetic peptide
HISTSSSNKYYADSVKG

SEQ ID NO: 479 - protein, 17 aa, Artificial Sequence
  Description of Artificial Sequence: Synthetic peptide
EGVGADYGDYYYYGMDV

SEQ ID NO: 480 - protein, 14 aa, Artificial Sequence
  Description of Artificial Sequence: Synthetic peptide
TGTSSDVGGYNYVS

SEQ ID NO: 481 - protein, 7 aa, Artificial Sequence
  Description of Artificial Sequence: Synthetic peptide
EVSNRPS

SEQ ID NO: 482 - protein, 10 aa, Artificial Sequence
  Description of Artificial Sequence: Synthetic peptide
SSYTSSYTYV

SEQ ID NO: 483 - protein, 7 aa, Artificial Sequence
  Description of Artificial Sequence: Synthetic peptide
GFTFNNY

SEQ ID NO: 484 - protein, 6 aa, Artificial Sequence
  Description of Artificial Sequence: Synthetic peptide
STSSSN

SEQ ID NO: 485 - protein, 16 aa, Artificial Sequence
  Description of Artificial Sequence: Synthetic peptide
EGVGADYGDYYYYGMD

SEQ ID NO: 486 - protein, 10 aa, Artificial Sequence
  Description of Artificial Sequence: Synthetic peptide
TSSDVGGYNY

SEQ ID NO: 487 - protein, 3 aa, Artificial Sequence
  Description of Artificial Sequence: Synthetic peptide
EVS

SEQ ID NO: 488 - protein, 7 aa, Artificial Sequence
  Description of Artificial Sequence: Synthetic peptide
YTSSYTY

SEQ ID NO: 489 - protein, 10 aa, Artificial Sequence
  Description of Artificial Sequence: Synthetic peptide
GFTFNNYNMN

SEQ ID NO: 490 - protein, 10 aa, Artificial Sequence
  Description of Artificial Sequence: Synthetic peptide
HISTSSSNKY

SEQ ID NO: 491-494 - skipped (4 entries)

SEQ ID NO: 495 - protein, 6 aa, Artificial Sequence
  Description of Artificial Sequence: Synthetic peptide
NNYNMN

SEQ ID NO: 496 - protein, 13 aa, Artificial Sequence
  Description of Artificial Sequence: Synthetic peptide
WVSHISTSSSNKY

SEQ ID NO: 497 - protein, 18 aa, Artificial Sequence
  Description of Artificial Sequence: Synthetic peptide
AREGVGADYGDYYYYGMD

SEQ ID NO: 498 - protein, 10 aa, Artificial Sequence
  Description of Artificial Sequence: Synthetic peptide
VGGYNYVSWY

SEQ ID NO: 499 - protein, 10 aa, Artificial Sequence
  Description of Artificial Sequence: Synthetic peptide
LMIYEVSNRP

SEQ ID NO: 500 - protein, 9 aa, Artificial Sequence
  Description of Artificial Sequence: Synthetic peptide
SSYTSSYTY

SEQ ID NO: 501 - protein, 8 aa, Artificial Sequence
  Description of Artificial Sequence: Synthetic peptide
GFTFNNYN

SEQ ID NO: 502 - protein, 8 aa, Artificial Sequence
  Description of Artificial Sequence: Synthetic peptide
ISTSSSNK

SEQ ID NO: 503 - protein, 19 aa, Artificial Sequence
  Description of Artificial Sequence: Synthetic peptide
AREGVGADYGDYYYYGMDV

SEQ ID NO: 504 - protein, 9 aa, Artificial Sequence
  Description of Artificial Sequence: Synthetic peptide
SSDVGGYNY

SEQ ID NO: 505-506 - skipped (2 entries)

SEQ ID NO: 507 - protein, 126 aa, Artificial Sequence
  Description of Artificial Sequence: Synthetic polypeptide
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCAREGVGADYGDYYYYGMDVWGQGTTVTVSS

SEQ ID NO: 508 - protein, 110 aa, Artificial Sequence
  Description of Artificial Sequence: Synthetic polypeptide
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVL

SEQ ID NO: 509-511 - skipped (3 entries)

SEQ ID NO: 512 - protein, 17 aa, Artificial Sequence
  Description of Artificial Sequence: Synthetic peptide
FISYDGSNKYYADSVKG

SEQ ID NO: 513 - protein, 13 aa, Artificial Sequence
  Description of Artificial Sequence: Synthetic peptide
RDNLRFLEWFMDV

SEQ ID NO: 514 - protein, 11 aa, Artificial Sequence
  Description of Artificial Sequence: Synthetic peptide
RASQSVRSNLA

SEQ ID NO: 515 - protein, 7 aa, Artificial Sequence
  Description of Artificial Sequence: Synthetic peptide
GASTRAT

SEQ ID NO: 516 - protein, 10 aa, Artificial Sequence
  Description of Artificial Sequence: Synthetic peptide
HQYNDWPPYT

SEQ ID NO: 517 - protein, 7 aa, Artificial Sequence
  Description of Artificial Sequence: Synthetic peptide
GFTFSTY

SEQ ID NO: 518 - protein, 6 aa, Artificial Sequence
  Description of Artificial Sequence: Synthetic peptide
SYDGSN

SEQ ID NO: 519 - protein, 12 aa, Artificial Sequence
  Description of Artificial Sequence: Synthetic peptide
RDNLRFLEWFMD

SEQ ID NO: 520 - protein, 7 aa, Artificial Sequence
  Description of Artificial Sequence: Synthetic peptide
SQSVRSN

SEQ ID NO: 521 - skipped

SEQ ID NO: 522 - protein, 7 aa, Artificial Sequence
  Description of Artificial Sequence: Synthetic peptide
YNDWPPY

SEQ ID NO: 523 - protein, 10 aa, Artificial Sequence
  Description of Artificial Sequence: Synthetic peptide
GFTFSTYGMH

SEQ ID NO: 524 - protein, 10 aa, Artificial Sequence
  Description of Artificial Sequence: Synthetic peptide
FISYDGSNKY

SEQ ID NO: 525-528 - skipped (4 entries)

SEQ ID NO: 529 - protein, 6 aa, Artificial Sequence
  Description of Artificial Sequence: Synthetic peptide
STYGMH

SEQ ID NO: 530 - protein, 13 aa, Artificial Sequence
  Description of Artificial Sequence: Synthetic peptide
WVAFISYDGSNKY

SEQ ID NO: 531 - protein, 14 aa, Artificial Sequence
  Description of Artificial Sequence: Synthetic peptide
AGRDNLRFLEWFMD

SEQ ID NO: 532 - protein, 7 aa, Artificial Sequence
  Description of Artificial Sequence: Synthetic peptide
RSNLAWY

SEQ ID NO: 533 - protein, 10 aa, Artificial Sequence
  Description of Artificial Sequence: Synthetic peptide
LLIYGASTRA

SEQ ID NO: 534 - protein, 9 aa, Artificial Sequence
  Description of Artificial Sequence: Synthetic peptide
HQYNDWPPY

SEQ ID NO: 535 - protein, 8 aa, Artificial Sequence
  Description of Artificial Sequence: Synthetic peptide
GFTFSTYG

SEQ ID NO: 536 - protein, 8 aa, Artificial Sequence
  Description of Artificial Sequence: Synthetic peptide
ISYDGSNK

SEQ ID NO: 537 - protein, 15 aa, Artificial Sequence
  Description of Artificial Sequence: Synthetic peptide
AGRDNLRFLEWFMDV

SEQ ID NO: 538 - protein, 6 aa, Artificial Sequence
  Description of Artificial Sequence: Synthetic peptide
QSVRSN

SEQ ID NO: 539-540 - skipped (2 entries)

SEQ ID NO: 541 - protein, 122 aa, Artificial Sequence
  Description of Artificial Sequence: Synthetic polypeptide
QVQLQESGGGVVQPGRSLRLSCAASGFTFSTYGMHWVRQAPGKGLEWVAFISYDGSNKYYADSVKGRFTISRDNSKHTLYLQMNSLRAEDTAVYYCAGRDNLRFLEWFMDVWGQGTTVTVSS

SEQ ID NO: 542 - protein, 108 aa, Artificial Sequence
  Description of Artificial Sequence: Synthetic polypeptide
EIVLTQSPGTLSVSPGERATLSCRASQSVRSNLAWYQQKPGQAPRLLIYGASTRATGIPARFSGSGSGTEFTLTISSLQSEDFAVYYCHQYNDWPPYTFGQGTKLEIK

SEQ ID NO: 543-544 - skipped (2 entries)

SEQ ID NO: 545 - protein, 5 aa, Artificial Sequence
  Description of Artificial Sequence: Synthetic peptide
SYGMN

SEQ ID NO: 546 - protein, 17 aa, Artificial Sequence
  Description of Artificial Sequence: Synthetic peptide
VTSYDGSNKYYADSVKG

SEQ ID NO: 547 - protein, 13 aa, Artificial Sequence
  Description of Artificial Sequence: Synthetic peptide
DPYSSSWNGAFDI

SEQ ID NO: 548 - protein, 11 aa, Artificial Sequence
  Description of Artificial Sequence: Synthetic peptide
GGNNIGSKSVH

SEQ ID NO: 549 - protein, 7 aa, Artificial Sequence
  Description of Artificial Sequence: Synthetic peptide
DDSDRPS

SEQ ID NO: 550 - protein, 11 aa, Artificial Sequence
  Description of Artificial Sequence: Synthetic peptide
QVWDSSTDHVV

SEQ ID NO: 551-552 - skipped (2 entries)

SEQ ID NO: 553 - protein, 12 aa, Artificial Sequence
  Description of Artificial Sequence: Synthetic peptide
DPYSSSWNGAFD

SEQ ID NO: 554 - protein, 7 aa, Artificial Sequence
  Description of Artificial Sequence: Synthetic peptide
NNIGSKS

SEQ ID NO: 555 - protein, 3 aa, Artificial Sequence
  Description of Artificial Sequence: Synthetic peptide
DDS

SEQ ID NO: 556 - protein, 8 aa, Artificial Sequence
  Description of Artificial Sequence: Synthetic peptide
WDSSTDHV

SEQ ID NO: 557 - protein, 10 aa, Artificial Sequence
  Description of Artificial Sequence: Synthetic peptide
GFTFSSYGMN

SEQ ID NO: 558 - protein, 10 aa, Artificial Sequence
  Description of Artificial Sequence: Synthetic peptide
VTSYDGSNKY

SEQ ID NO: 559-562 - skipped (4 entries)

SEQ ID NO: 563 - protein, 6 aa, Artificial Sequence
  Description of Artificial Sequence: Synthetic peptide
SSYGMN

SEQ ID NO: 564 - protein, 13 aa, Artificial Sequence
  Description of Artificial Sequence: Synthetic peptide
WVAVTSYDGSNKY

SEQ ID NO: 565 - protein, 14 aa, Artificial Sequence
  Description of Artificial Sequence: Synthetic peptide
ARDPYSSSWNGAFD

SEQ ID NO: 566 - protein, 7 aa, Artificial Sequence
  Description of Artificial Sequence: Synthetic peptide
SKSVHWY

SEQ ID NO: 567 - protein, 10 aa, Artificial Sequence
  Description of Artificial Sequence: Synthetic peptide
LVVYDDSDRP

SEQ ID NO: 568 - protein, 10 aa, Artificial Sequence
  Description of Artificial Sequence: Synthetic peptide
QVWDSSTDHV

SEQ ID NO: 569 - skipped

SEQ ID NO: 570 - protein, 8 aa, Artificial Sequence
  Description of Artificial Sequence: Synthetic peptide
TSYDGSNK

SEQ ID NO: 571 - protein, 15 aa, Artificial Sequence
  Description of Artificial Sequence: Synthetic peptide
ARDPYSSSWNGAFDI

SEQ ID NO: 572 - protein, 6 aa, Artificial Sequence
  Description of Artificial Sequence: Synthetic peptide
NIGSKS

SEQ ID NO: 573-574 - skipped (2 entries)

SEQ ID NO: 575 - protein, 122 aa, Artificial Sequence
  Description of Artificial Sequence: Synthetic polypeptide
EVQLVESGGGVVQPGRSLRLSCAASGFTFSSYGMNWVRQAPGKGLEWVAVTSYDGSNKYYADSVKGRFTISRDISKNTLYLQMSSLRAEDTAVYYCARDPYSSSWNGAFDIWGPGTMVTVSS

SEQ ID NO: 576 - protein, 108 aa, Artificial Sequence
  Description of Artificial Sequence: Synthetic polypeptide
SYELTQPPSVSVAPGQTARITCGGNNIGSKSVHWYQQKPGQAPVLVVYDDSDRPSGIPERFSGSNSGNTATLTISRVEAGDEADYYCQVWDSSTDHVVFGGGTKLTVL

SEQ ID NO: 577-586 - skipped (10 entries)

SEQ ID NO: 587 - protein, 15 aa, Artificial Sequence
  Description of Artificial Sequence: Synthetic peptide
ALRDGNNWDYFNGMD

SEQ ID NO: 588-614 - skipped (27 entries)

SEQ ID NO: 615 - protein, 17 aa, Artificial Sequence
  Description of Artificial Sequence: Synthetic peptide
DGVGADYGDYYYYGMDV

SEQ ID NO: 616-620 - skipped (5 entries)

SEQ ID NO: 621 - protein, 16 aa, Artificial Sequence
  Description of Artificial Sequence: Synthetic peptide
DGVGADYGDYYYYGMD

SEQ ID NO: 622-632 - skipped (11 entries)

SEQ ID NO: 633 - protein, 18 aa, Artificial Sequence
  Description of Artificial Sequence: Synthetic peptide
ARDGVGADYGDYYYYGMD

SEQ ID NO: 634-638 - skipped (5 entries)

SEQ ID NO: 639 - protein, 19 aa, Artificial Sequence
  Description of Artificial Sequence: Synthetic peptide
ARDGVGADYGDYYYYGMDV

SEQ ID NO: 640-642 - skipped (3 entries)

SEQ ID NO: 643 - protein, 126 aa, Artificial Sequence
  Description of Artificial Sequence: Synthetic polypeptide
EVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCARDGVGADYGDYYYYGMDVWGQGTTVTVSS

SEQ ID NO: 644-646 - skipped (3 entries)

SEQ ID NO: 647 - protein, 5 aa, Artificial Sequence
  Description of Artificial Sequence: Synthetic peptide
GYGMH

SEQ ID NO: 648 - protein, 17 aa, Artificial Sequence
  Description of Artificial Sequence: Synthetic peptide
VMSYDGSNRFYVDSVRG

SEQ ID NO: 649 - protein, 13 aa, Artificial Sequence
  Description of Artificial Sequence: Synthetic peptide
DTVWGSHPDAFDI

SEQ ID NO: 650 - protein, 11 aa, Artificial Sequence
  Description of Artificial Sequence: Synthetic peptide
GGNNSGSKSVH

SEQ ID NO: 651 - skipped

SEQ ID NO: 652 - protein, 11 aa, Artificial Sequence
  Description of Artificial Sequence: Synthetic peptide
QVWDSSSDHGV

SEQ ID NO: 653 - protein, 7 aa, Artificial Sequence
  Description of Artificial Sequence: Synthetic peptide
GFSFSGY

SEQ ID NO: 654 - skipped

SEQ ID NO: 655 - protein, 12 aa, Artificial Sequence
  Description of Artificial Sequence: Synthetic peptide
DTVWGSHPDAFD

SEQ ID NO: 656 - protein, 7 aa, Artificial Sequence
  Description of Artificial Sequence: Synthetic peptide
NNSGSKS

SEQ ID NO: 657 - skipped

SEQ ID NO: 658 - protein, 8 aa, Artificial Sequence
  Description of Artificial Sequence: Synthetic peptide
WDSSSDHG

SEQ ID NO: 659 - protein, 10 aa, Artificial Sequence
  Description of Artificial Sequence: Synthetic peptide
GFSFSGYGMH

SEQ ID NO: 660 - protein, 10 aa, Artificial Sequence
  Description of Artificial Sequence: Synthetic peptide
VMSYDGSNRF

SEQ ID NO: 661-664 - skipped (4 entries)

SEQ ID NO: 665 - protein, 6 aa, Artificial Sequence
  Description of Artificial Sequence: Synthetic peptide
SGYGMH

SEQ ID NO: 666 - protein, 13 aa, Artificial Sequence
  Description of Artificial Sequence: Synthetic peptide
WVAVMSYDGSNRF

SEQ ID NO: 667 - protein, 14 aa, Artificial Sequence
  Description of Artificial Sequence: Synthetic peptide
ARDTVWGSHPDAFD

SEQ ID NO: 668-669 - skipped (2 entries)

SEQ ID NO: 670 - protein, 10 aa, Artificial Sequence
  Description of Artificial Sequence: Synthetic peptide
QVWDSSSDHG

SEQ ID NO: 671 - protein, 8 aa, Artificial Sequence
  Description of Artificial Sequence: Synthetic peptide
GFSFSGYG

SEQ ID NO: 672 - protein, 8 aa, Artificial Sequence
  Description of Artificial Sequence: Synthetic peptide
MSYDGSNR

SEQ ID NO: 673 - protein, 15 aa, Artificial Sequence
  Description of Artificial Sequence: Synthetic peptide
ARDTVWGSHPDAFDI

SEQ ID NO: 674 - protein, 6 aa, Artificial Sequence
  Description of Artificial Sequence: Synthetic peptide
NSGSKS

SEQ ID NO: 675-676 - skipped (2 entries)

SEQ ID NO: 677 - protein, 122 aa, Artificial Sequence
  Description of Artificial Sequence: Synthetic polypeptide
QVQLVESGGGEVQPGRSLRLSCAASGFSFSGYGMHWVRQAPGKGLEWVAVMSYDGSNRFYVDSVRGRFSISRDNSKNTLYLQMNSLRPEDTAVYYCARDTVWGSHPDAFDIWGQGTVVTVSS

SEQ ID NO: 678 - protein, 108 aa, Artificial Sequence
  Description of Artificial Sequence: Synthetic polypeptide
QSVLTQPPSVSVAPGQTARITCGGNNSGSKSVHWYQQKPGQAPVLVVYDDSDRPSGIPERFSGSNSGNTATLTISRVEAGDEADYYCQVWDSSSDHGVFGGGTKLTVL

SEQ ID NO: 679-681 - skipped (3 entries)

SEQ ID NO: 682 - protein, 17 aa, Artificial Sequence
  Description of Artificial Sequence: Synthetic peptide
IIYYDGSNKYYADSVKG

SEQ ID NO: 683-687 - skipped (5 entries)

SEQ ID NO: 688 - protein, 6 aa, Artificial Sequence
  Description of Artificial Sequence: Synthetic peptide
YYDGSN

SEQ ID NO: 689-693 - skipped (5 entries)

SEQ ID NO: 694 - protein, 10 aa, Artificial Sequence
  Description of Artificial Sequence: Synthetic peptide
IIYYDGSNKY

SEQ ID NO: 695-699 - skipped (5 entries)

SEQ ID NO: 700 - protein, 13 aa, Artificial Sequence
  Description of Artificial Sequence: Synthetic peptide
WVAIIYYDGSNKY

SEQ ID NO: 701-705 - skipped (5 entries)

SEQ ID NO: 706 - protein, 8 aa, Artificial Sequence
  Description of Artificial Sequence: Synthetic peptide
IYYDGSNK

SEQ ID NO: 707-710 - skipped (4 entries)

SEQ ID NO: 711 - protein, 120 aa, Artificial Sequence
  Description of Artificial Sequence: Synthetic polypeptide
QVQLVESGGGVVQPGRSLRLSCAASGFTFSSYNMNWVRQAPGKGLEWVAIIYYDGSNKYYADSVKGRFTISRDISKNTLYLQMNSLRAEDTAVYYCARERGRDYYGMDVWGQGTTVTVSS

SEQ ID NO: 712-714 - skipped (3 entries)

SEQ ID NO: 715 - protein, 5 aa, Artificial Sequence
  Description of Artificial Sequence: Synthetic peptide
SYYWS

SEQ ID NO: 716 - protein, 16 aa, Artificial Sequence
  Description of Artificial Sequence: Synthetic peptide
RIYSSGSTNYNPSLKS

SEQ ID NO: 717 - protein, 10 aa, Artificial Sequence
  Description of Artificial Sequence: Synthetic peptide
VGVWPGAFDI

SEQ ID NO: 718 - skipped

SEQ ID NO: 719 - protein, 7 aa, Artificial Sequence
  Description of Artificial Sequence: Synthetic peptide
SSNQRPS

SEQ ID NO: 720 - protein, 11 aa, Artificial Sequence
  Description of Artificial Sequence: Synthetic peptide
AAWDDSLNGVV

SEQ ID NO: 721 - protein, 7 aa, Artificial Sequence
  Description of Artificial Sequence: Synthetic peptide
GGSIISY

SEQ ID NO: 722 - protein, 5 aa, Artificial Sequence
  Description of Artificial Sequence: Synthetic peptide
YSSGS

SEQ ID NO: 723 - protein, 9 aa, Artificial Sequence
  Description of Artificial Sequence: Synthetic peptide
VGVWPGAFD

SEQ ID NO: 724 - skipped

SEQ ID NO: 725 - protein, 3 aa, Artificial Sequence
  Description of Artificial Sequence: Synthetic peptide
SSN

SEQ ID NO: 726 - protein, 8 aa, Artificial Sequence
  Description of Artificial Sequence: Synthetic peptide
WDDSLNGV

SEQ ID NO: 727 - protein, 10 aa, Artificial Sequence
  Description of Artificial Sequence: Synthetic peptide
GGSIISYYWS

SEQ ID NO: 728 - protein, 9 aa, Artificial Sequence
  Description of Artificial Sequence: Synthetic peptide
RIYSSGSTN

SEQ ID NO: 729-732 - skipped (4 entries)

SEQ ID NO: 733 - protein, 6 aa, Artificial Sequence
  Description of Artificial Sequence: Synthetic peptide
ISYYWS

SEQ ID NO: 734 - protein, 12 aa, Artificial Sequence
  Description of Artificial Sequence: Synthetic peptide
WIGRIYSSGSTN

SEQ ID NO: 735 - protein, 11 aa, Artificial Sequence
  Description of Artificial Sequence: Synthetic peptide
AKVGVWPGAFD

SEQ ID NO: 736 - protein, 9 aa, Artificial Sequence
  Description of Artificial Sequence: Synthetic peptide
IGSNTVNWY

SEQ ID NO: 737 - protein, 10 aa, Artificial Sequence
  Description of Artificial Sequence: Synthetic peptide
LLIYSSNQRP

SEQ ID NO: 738 - protein, 10 aa, Artificial Sequence
  Description of Artificial Sequence: Synthetic peptide
AAWDDSLNGV

SEQ ID NO: 739 - protein, 8 aa, Artificial Sequence
  Description of Artificial Sequence: Synthetic peptide
GGSIISYY

SEQ ID NO: 740 - protein, 7 aa, Artificial Sequence
  Description of Artificial Sequence: Synthetic peptide
IYSSGST

SEQ ID NO: 741 - protein, 12 aa, Artificial Sequence
  Description of Artificial Sequence: Synthetic peptide
AKVGVWPGAFDI

SEQ ID NO: 742-744 - skipped (3 entries)

SEQ ID NO: 745 - protein, 118 aa, Artificial Sequence
  Description of Artificial Sequence: Synthetic polypeptide
EVQLLESGPGLVKPSETLSLTCTVSGGSIISYYWSWIRQPAGKGLEWIGRIYSSGSTNYNPSLKSRVTMSVDTSKNQFSLKLSSVTAADTAVYYCAKVGVWPGAFDIWGQGTMVTVSS

SEQ ID NO: 746 - protein, 110 aa, Artificial Sequence
  Description of Artificial Sequence: Synthetic polypeptide
QSVLTQPPSASGTPGQRVTISCSGSSSNIGSNTVNWYQQLPGTAPKLLIYSSNQRPSGVPDRFSGSKSGTSASLAISGLQSEDEADYYCAAWDDSLNGVVFGGGTKLTVL

SEQ ID NO: 747-749 - skipped (3 entries)

SEQ ID NO: 750 - protein, 17 aa, Artificial Sequence
  Description of Artificial Sequence: Synthetic peptide
VISYDGSNKYYADSVKG

SEQ ID NO: 751 - protein, 15 aa, Artificial Sequence
  Description of Artificial Sequence: Synthetic peptide
DRIWGSRGYYYGMDV

SEQ ID NO: 752 - protein, 14 aa, Artificial Sequence
  Description of Artificial Sequence: Synthetic peptide
TGASSDVGGYNYVS

SEQ ID NO: 753 - skipped

SEQ ID NO: 754 - protein, 10 aa, Artificial Sequence
  Description of Artificial Sequence: Synthetic peptide
SSYTITSTLV

SEQ ID NO: 755-756 - skipped (2 entries)

SEQ ID NO: 757 - protein, 14 aa, Artificial Sequence
  Description of Artificial Sequence: Synthetic peptide
DRIWGSRGYYYGMD

SEQ ID NO: 758 - protein, 10 aa, Artificial Sequence
  Description of Artificial Sequence: Synthetic peptide
ASSDVGGYNY

SEQ ID NO: 759 - skipped

SEQ ID NO: 760 - protein, 7 aa, Artificial Sequence
  Description of Artificial Sequence: Synthetic peptide
YTITSTL

SEQ ID NO: 761 - skipped

SEQ ID NO: 762 - protein, 10 aa, Artificial Sequence
  Description of Artificial Sequence: Synthetic peptide
VISYDGSNKY

SEQ ID NO: 763-767 - skipped (5 entries)

SEQ ID NO: 768 - protein, 13 aa, Artificial Sequence
  Description of Artificial Sequence: Synthetic peptide
WVAVISYDGSNKY

SEQ ID NO: 769 - protein, 16 aa, Artificial Sequence
  Description of Artificial Sequence: Synthetic peptide
ARDRIWGSRGYYYGMD

SEQ ID NO: 770-771 - skipped (2 entries)

SEQ ID NO: 772 - protein, 9 aa, Artificial Sequence
  Description of Artificial Sequence: Synthetic peptide
SSYTITSTL

SEQ ID NO: 773-774 - skipped (2 entries)

SEQ ID NO: 775 - protein, 17 aa, Artificial Sequence
  Description of Artificial Sequence: Synthetic peptide
ARDRIWGSRGYYYGMDV

SEQ ID NO: 776-778 - skipped (3 entries)

SEQ ID NO: 779 - protein, 124 aa, Artificial Sequence
  Description of Artificial Sequence: Synthetic polypeptide
EVQLVESGGGVVQPGRSLRLSCAASGFSFSGYGMHWVRQAPGKGLEWVAVISYDGSNKYYADSVKGRFTISRDNSKNTLYLQMNSLRVEDTAVYYCARDRIWGSRGYYYGMDVWGQGTTVTVSS

SEQ ID NO: 780 - protein, 110 aa, Artificial Sequence
  Description of Artificial Sequence: Synthetic polypeptide
QSALTQPASVSGSPGQSITISCTGASSDVGGYNYVSWYQQHPGKAPKLMIYEVSNRPSGVSNRFSGSKSGNTASLTISGLQAEDEADYYCSSYTITSTLVFGGGTKLTVL

SEQ ID NO: 781-782 - skipped (2 entries)

SEQ ID NO: 783 - protein, 5 aa, Artificial Sequence
  Description of Artificial Sequence: Synthetic peptide
GYGLH

SEQ ID NO: 784 - protein, 17 aa, Artificial Sequence
  Description of Artificial Sequence: Synthetic peptide
LISYDGSNKYYADSVKG

SEQ ID NO: 785 - protein, 13 aa, Artificial Sequence
  Description of Artificial Sequence: Synthetic peptide
TTVSDPYYYGMDV

SEQ ID NO: 786-787 - skipped (2 entries)

SEQ ID NO: 788 - protein, 11 aa, Artificial Sequence
  Description of Artificial Sequence: Synthetic peptide
QVWDSSSDHVV

SEQ ID NO: 789-790 - skipped (2 entries)

SEQ ID NO: 791 - protein, 12 aa, Artificial Sequence
  Description of Artificial Sequence: Synthetic peptide
TTVSDPYYYGMD

SEQ ID NO: 792-793 - skipped (2 entries)

SEQ ID NO: 794 - protein, 8 aa, Artificial Sequence
  Description of Artificial Sequence: Synthetic peptide
WDSSSDHV

SEQ ID NO: 795 - protein, 10 aa, Artificial Sequence
  Description of Artificial Sequence: Synthetic peptide
GFSFSGYGLH

SEQ ID NO: 796 - protein, 10 aa, Artificial Sequence
  Description of Artificial Sequence: Synthetic peptide
LISYDGSNKY

SEQ ID NO: 797-800 - skipped (4 entries)

SEQ ID NO: 801 - protein, 6 aa, Artificial Sequence
  Description of Artificial Sequence: Synthetic peptide
SGYGLH

SEQ ID NO: 802 - protein, 13 aa, Artificial Sequence
  Description of Artificial Sequence: Synthetic peptide
WVTLISYDGSNKY

SEQ ID NO: 803 - protein, 14 aa, Artificial Sequence
  Description of Artificial Sequence: Synthetic peptide
AKTTVSDPYYYGMD

SEQ ID NO: 804-805 - skipped (2 entries)

SEQ ID NO: 806 - protein, 10 aa, Artificial Sequence
  Description of Artificial Sequence: Synthetic peptide
QVWDSSSDHV

SEQ ID NO: 807-808 - skipped (2 entries)

SEQ ID NO: 809 - protein, 15 aa, Artificial Sequence
  Description of Artificial Sequence: Synthetic peptide
AKTTVSDPYYYGMDV

SEQ ID NO: 810-812 - skipped (3 entries)

SEQ ID NO: 813 - protein, 122 aa, Artificial Sequence
  Description of Artificial Sequence: Synthetic polypeptide
QVQLQESGGDVVQPGRSLRLSCAASGFSFSGYGLHWVRQAPGRGLEWVTLISYDGSNKYYADSVKGRFTISRDNSKNTLYLQMNSLRAEDTAVYYCAKTTVSDPYYYGMDVWGQGTTVTVSS

SEQ ID NO: 814 - protein, 108 aa, Artificial Sequence
  Description of Artificial Sequence: Synthetic polypeptide
SYELTQPPSVSVAPGQTARITCGGNNIGSKSVHWYQQKPGQAPVLVVYDDSDRPSGIPERFSGTNSGNTATLTISRAEAGDEADYYCQVWDSSSDHVVFGGGTKLTVL

SEQ ID NO: 815-816 - skipped (2 entries)

SEQ ID NO: 817 - protein, 7 aa, Artificial Sequence
  Description of Artificial Sequence: Synthetic peptide
NNNAAWS

SEQ ID NO: 818 - protein, 18 aa, Artificial Sequence
  Description of Artificial Sequence: Synthetic peptide
RTYYRSKWLYDYAVSVKS

SEQ ID NO: 819 - protein, 8 aa, Artificial Sequence
  Description of Artificial Sequence: Synthetic peptide
GYSSSFDY

SEQ ID NO: 820 - protein, 14 aa, Artificial Sequence
  Description of Artificial Sequence: Synthetic peptide
TGTSSNIGTYKFVS

SEQ ID NO: 821 - protein, 7 aa, Artificial Sequence
  Description of Artificial Sequence: Synthetic peptide
EVSKRPS

SEQ ID NO: 822 - protein, 10 aa, Artificial Sequence
  Description of Artificial Sequence: Synthetic peptide
VSYAGSGTLL

SEQ ID NO: 823 - protein, 9 aa, Artificial Sequence
  Description of Artificial Sequence: Synthetic peptide
GDSVFNNNA

SEQ ID NO: 824 - protein, 7 aa, Artificial Sequence
  Description of Artificial Sequence: Synthetic peptide
YYRSKWL

SEQ ID NO: 825 - protein, 6 aa, Artificial Sequence
  Description of Artificial Sequence: Synthetic peptide
YSSSFD

SEQ ID NO: 826 - protein, 10 aa, Artificial Sequence
  Description of Artificial Sequence: Synthetic peptide
TSSNIGTYKF

SEQ ID NO: 827 - skipped

SEQ ID NO: 828 - protein, 7 aa, Artificial Sequence
  Description of Artificial Sequence: Synthetic peptide
YAGSGTL

SEQ ID NO: 829 - protein, 12 aa, Artificial Sequence
  Description of Artificial Sequence: Synthetic peptide
GDSVFNNNAAWS

SEQ ID NO: 830 - protein, 11 aa, Artificial Sequence
  Description of Artificial Sequence: Synthetic peptide
RTYYRSKWLYD

SEQ ID NO: 831-834 - skipped (4 entries)

SEQ ID NO: 835 - protein, 8 aa, Artificial Sequence
  Description of Artificial Sequence: Synthetic peptide
FNNNAAWS

SEQ ID NO: 836 - protein, 14 aa, Artificial Sequence
  Description of Artificial Sequence: Synthetic peptide
WLGRTYYRSKWLYD

SEQ ID NO: 837 - protein, 9 aa, Artificial Sequence
  Description of Artificial Sequence: Synthetic peptide
ARGYSSSFD

SEQ ID NO: 838 - protein, 10 aa, Artificial Sequence
  Description of Artificial Sequence: Synthetic peptide
IGTYKFVSWY

SEQ ID NO: 839 - protein, 10 aa, Artificial Sequence
  Description of Artificial Sequence: Synthetic peptide
VLLYEVSKRP

SEQ ID NO: 840 - protein, 9 aa, Artificial Sequence
  Description of Artificial Sequence: Synthetic peptide
VSYAGSGTL

SEQ ID NO: 841 - protein, 10 aa, Artificial Sequence
  Description of Artificial Sequence: Synthetic peptide
GDSVFNNNAA

SEQ ID NO: 842 - protein, 9 aa, Artificial Sequence
  Description of Artificial Sequence: Synthetic peptide
TYYRSKWLY

SEQ ID NO: 843 - protein, 10 aa, Artificial Sequence
  Description of Artificial Sequence: Synthetic peptide
ARGYSSSFDY

SEQ ID NO: 844 - protein, 9 aa, Artificial Sequence
  Description of Artificial Sequence: Synthetic peptide
SSNIGTYKF

SEQ ID NO: 845-846 - skipped (2 entries)

SEQ ID NO: 847 - protein, 120 aa, Artificial Sequence
  Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 848 - protein, 110 aa, Artificial Sequence
  Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 849 - protein, 450 aa, Artificial Sequence
  Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 850 - protein, 216 aa, Artificial Sequence
  Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 851-882 - skipped (32 entries)

SEQ ID NO: 883 - protein, 449 aa, Artificial Sequence
  Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 884-906 - skipped (23 entries)

SEQ ID NO: 907 - protein, 10 aa, Artificial Sequence
  Description of Artificial Sequence: Synthetic peptide
VMIYEVSKRP

SEQ ID NO: 908-914 - skipped (7 entries)

SEQ ID NO: 915 - protein, 120 aa, Artificial Sequence
  Description of Artificial Sequence: Synthetic polypeptide
QVQLQQSGPGLVKPSQTLSLTCAISGDSVFNNNAAWSWIRQSPSRGLEWLGRTYYRSKWLYDYAVSVKSRITINPDTSKNQFSLQLNSVTPEDTAVYYCARGYSSSFDYWGQGTLVTVSS

SEQ ID NO: 916 - protein, 110 aa, Artificial Sequence
  Description of Artificial Sequence: Synthetic polypeptide
QSALTQPASVSGSPGQSITISCTGTSSNIGTYKFVSWYQQHPGKAPKVMIYEVSKRPSGVSNRFSGSKSGNTASLTISGLQAEDEADYYCVSYAGSGTLLFGGGTKLTVL

SEQ ID NO: 917 - protein, 450 aa, Artificial Sequence
  Description of Artificial Sequence: Synthetic polypeptide
QVQLQQSGPGLVKPSQTLSLTCAISGDSVFNNNAAWSWIRQSPSRGLEWLGRTYYRSKWLYDYAVSVKSRITINPDTSKNQFSLQLNSVTPEDTAVYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 918 - protein, 216 aa, Artificial Sequence
  Description of Artificial Sequence: Synthetic polypeptide
QSALTQPASVSGSPGQSITISCTGTSSNIGTYKFVSWYQQHPGKAPKVMIYEVSKRPSGVSNRFSGSKSGNTASLTISGLQAEDEADYYCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 919-950 - skipped (32 entries)

SEQ ID NO: 951 - protein, 449 aa, Artificial Sequence
  Description of Artificial Sequence: Synthetic polypeptide
QVQLQQSGPGLVKPSQTLSLTCAISGDSVFNNNAAWSWIRQSPSRGLEWLGRTYYRSKWLYDYAVSVKSRITINPDTSKNQFSLQLNSVTPEDTAVYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 952-953 - skipped (2 entries)

SEQ ID NO: 954 - protein, 17 aa, Artificial Sequence
  Description of Artificial Sequence: Synthetic peptide
SISTSSNYIYYADSVKG

SEQ ID NO: 955 - protein, 7 aa, Artificial Sequence
  Description of Artificial Sequence: Synthetic peptide
GWGPFDY

SEQ ID NO: 956 - protein, 11 aa, Artificial Sequence
  Description of Artificial Sequence: Synthetic peptide
RARQSIGTAIH

SEQ ID NO: 957 - protein, 7 aa, Artificial Sequence
  Description of Artificial Sequence: Synthetic peptide
YASESIS

SEQ ID NO: 958 - protein, 9 aa, Artificial Sequence
  Description of Artificial Sequence: Synthetic peptide
QQSGSWPYT

SEQ ID NO: 959 - skipped

SEQ ID NO: 960 - protein, 6 aa, Artificial Sequence
  Description of Artificial Sequence: Synthetic peptide
STSSNY

SEQ ID NO: 961 - protein, 6 aa, Artificial Sequence
  Description of Artificial Sequence: Synthetic peptide
GWGPFD

SEQ ID NO: 962 - protein, 7 aa, Artificial Sequence
  Description of Artificial Sequence: Synthetic peptide
RQSIGTA

SEQ ID NO: 963 - protein, 3 aa, Artificial Sequence
  Description of Artificial Sequence: Synthetic peptide
YAS

SEQ ID NO: 964 - protein, 6 aa, Artificial Sequence
  Description of Artificial Sequence: Synthetic peptide
SGSWPY

SEQ ID NO: 965 - skipped

SEQ ID NO: 966 - protein, 10 aa, Artificial Sequence
  Description of Artificial Sequence: Synthetic peptide
SISTSSNYIY

SEQ ID NO: 967-971 - skipped (5 entries)

SEQ ID NO: 972 - protein, 13 aa, Artificial Sequence
  Description of Artificial Sequence: Synthetic peptide
WVSSISTSSNYIY

SEQ ID NO: 973 - protein, 8 aa, Artificial Sequence
  Description of Artificial Sequence: Synthetic peptide
TRGWGPFD

SEQ ID NO: 974 - protein, 7 aa, Artificial Sequence
  Description of Artificial Sequence: Synthetic peptide
GTAIHWY

SEQ ID NO: 975 - protein, 10 aa, Artificial Sequence
  Description of Artificial Sequence: Synthetic peptide
LLIKYASESI

SEQ ID NO: 976 - protein, 8 aa, Artificial Sequence
  Description of Artificial Sequence: Synthetic peptide
QQSGSWPY

SEQ ID NO: 977 - skipped

SEQ ID NO: 978 - protein, 8 aa, Artificial Sequence
  Description of Artificial Sequence: Synthetic peptide
ISTSSNYI

SEQ ID NO: 979 - protein, 9 aa, Artificial Sequence
  Description of Artificial Sequence: Synthetic peptide
TRGWGPFDY

SEQ ID NO: 980 - protein, 6 aa, Artificial Sequence
  Description of Artificial Sequence: Synthetic peptide
QSIGTA

SEQ ID NO: 981-982 - skipped (2 entries)

SEQ ID NO: 983 - protein, 116 aa, Artificial Sequence
  Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 984 - protein, 107 aa, Artificial Sequence
  Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 985 - protein, 445 aa, Artificial Sequence
  Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 986 - protein, 214 aa, Artificial Sequence
  Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 987-1018 - skipped (32 entries)

SEQ ID NO: 1019 - protein, 446 aa, Artificial Sequence
  Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 1020 - skipped

SEQ ID NO: 1021 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 1022 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 1023 - protein, 713 aa, Artificial Sequence
  Description of Artificial Sequence: Synthetic polypeptide
HHHHHHKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 1024 - protein, 730 aa, Artificial Sequence
  Description of Artificial Sequence: Synthetic polypeptide
GLNDIFEAQKIEWHEHHHHHHGSKSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPDDYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGMLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 1025 - protein, 731 aa, Artificial Sequence
  Description of Artificial Sequence: Synthetic polypeptide
GLNDIFEAQKIEWHEHHHHHHGSKPSNEATGNVSHSGMKKEFLHELKAENIKKFLYNFTRTPHLAGTQNNFELAKQIHDQWKEFGLDLVELSHYDVLLSYPNKTHPNYISIINEDGNEIFKTSLSEQPPPGYENISDVVPPYSAFSPQGTPEGDLVYVNYARTEDFFKLEREMKISCSGKIVIARYGKVFRGNMVKNAQLAGAKGMILYSDPADYFVPAVKSYPDGWNLPGGGVQRGNVLNLNGAGDPLTPGYPANEHAYRHELTNAVGLPSIPVHPIGYDDAQKLLEHMGGPAPPDSSWKGGLKVPYNVGPGFAGNFSTQKVKMHIHSYTKVTRIYNVIGTLKGALEPDRYVILGGHRDAWVFGGIDPQSGAAVVHEIVRSFGTLKKKGRRPRRTILFASWDAEEFGLLGSTEWAEEHSRLLQERGVAYINADSSIEGNYTLRVDCTPLMYSLVYNLTKELQSPDEGFEGKSLYDSWKEKSPSPEFIGMPRISKLGSGNDFEVFFQRLGIASGRARYTKNWKTNKVSSYPLYHSVYETYELVVKFYDPTFKYHLTVAQVRGAMVFELANSIVLPFDCQSYAVALKKYADTIYNISMKHPQEMKAYMISFDSLFSAVNNFTDVASKFNQRLQELDKSNPILLRIMNDQLMYLERAFIDPLGLPGRPFYRHIIYAPSSHNKYAGESFPGIYDALFDISSKVNASKAWNEVKRQISIATFTVQAAAETLREVA

SEQ ID NO: 1026 - DNA, 366 bp, Artificial Sequence
  Description of Artificial Sequence: Synthetic polynucleotide
gaggtgcaactggtcgaatctggagggggactggttaagcctgggggtagcttgcgtcttagctgtgccgctagcggcttcaccttctcacgttacaatatgaattgggttcgacaggcccctggcaaaggtcttgagtgggtttcatccattaactcaaattcacgatatatctactacgcagattctgttaagggtcgattcactatatctcgagacagtgccaagaattccttgtacttgcagatgaatagcttgagggccgaagatactgccgtgtactactgtgcaaagacaatgggagattattattactattacggcatggacgtgtgggggcaggggacaacagtcaccgtaagcagt

SEQ ID NO: 1027 - skipped

SEQ ID NO: 1028 - DNA, 375 bp, Artificial Sequence
  Description of Artificial Sequence: Synthetic polynucleotide
gaggtgcaattggttgagagtggcgggggggtcgttcaaccaggtcgcagcttgcgactctcttgtgccgccagcggtttcacattcataacttacgggatgcactgggtgcgacaggcacccggaaagggactggagtgggttgctgtggtgtctttcgatgagtctaataagtactatgcagactctgttaaaggccgatttactattagccgagacaactctaaaaatactctgtacctccagatgaatagtttgcgagcagaagatacagcagtttattattgtgccagggctcttagggacggtaataattgggattactttaacggcatggacgtatgggggcaaggcactaccgttactgtatcatcc

SEQ ID NO: 1029 - skipped

SEQ ID NO: 1030 - DNA, 372 bp, Artificial Sequence
  Description of Artificial Sequence: Synthetic polynucleotide
caagtgcagttggttgagtcaggtggaggagtggtccagcctggtcggagcctcagattgtcctgcgtggcaagcggattcaccttttcttcttacggtattcactgggtacggcaagcaccagggaaagggttggagtgggtcgctgtgatttggtacgacgggtcaaataagtactacgccgattccgtgaagggtcgctttacaatctctagggataactcaaaaaacacactgtacctgcaaatgaatagtctccgtgcagaagacacagcagtatattattctgtgcgcggagtagggcccacttcttattattacaactacggtatggatgtatggggccagggcactaccgttactgtatcttcc

SEQ ID NO: 1031 - skipped

SEQ ID NO: 1032 - DNA, 363 bp, Artificial Sequence
  Description of Artificial Sequence: Synthetic polynucleotide
gaggtacaacttgtggaaagtggaggcggtcttgtccaacctggaggatctctccgattgagttgcgcagccagcgggtttactttttcttcatacgccatgtcctgggtgcggcaagcaccaggtaaaggacttgagtgggtatctgctatttcaggggggataggctcaacatactatgctgatagcgtgaaaggtaggttcaccatttcccgtgacaatagtaaaaacacattgtggctccaaatgaacagccttagggctgaagacaccgctgtttactactgcgctaaagacggtgtaggggcaactccctattacttcgattattggggacaaggaaccttggtaacagtttcaagc

SEQ ID NO: 1033 - skipped

SEQ ID NO: 1034 - DNA, 363 bp, Artificial Sequence
  Description of Artificial Sequence: Synthetic polynucleotide
gaagtccagttggtagaatctggaggcgggctggtacagcctggcggttccttgcgcctctcatgtgccgcaagcgggtttaccttcagctcttacgcaatgtcatgggtgcgtcaggcccctggaaaaggtctcgagtgggtcagtgccatttctgggggctccggctccacctactacgcagattcagttaaagggagatttacaatctcaagagataacagtaaaaacaccctctacctccagatgaactcacttcgagctgaggattcagcagtatattactgtgctaaagacggtgtaggtgcaactccctactatttcgactattggggccaagggactttggtgacagtaagtagt

SEQ ID NO: 1035 - skipped

SEQ ID NO: 1036 - DNA, 363 bp, Artificial Sequence
  Description of Artificial Sequence: Synthetic polynucleotide
gaggtgcagcttgtagaatcagggggaggactggtgcagccaggcggatcattgagactgagttgtactgccagcggattcatattctccagttatgccatgagctgggtgcggcaggcacctggaaaagggcttgagtgggtctctgccataagcggtggatacggtgctccatactatgctgacaccgtaaaagggcggttcaccatctcccgagacaatagtaagaacaccctgtatctccagatgaactccctccgcgctgaagatacagccgtttattactgcgccaaagatggagtcggggccactccatactacttcgatgattggggacaagggatcctcgtcactgttagctca

SEQ ID NO: 1037 - DNA, 333 bp, Artificial Sequence
  Description of Artificial Sequence: Synthetic polynucleotide
caatccgtgcttacacaaccaccatcagtaagcggggctccaggccaaagagtaaccatctcatgcactggttccagcttcaaccttggtgccggatatgatgtccattggtatcaacaagtccctggaaccgtcccaaaactcctgatctacgataattcaaatcgaccttccggagtgccagatcggttcagtggatctaaaagcggcaccagtgcctctcttgccattactggattgcaagctgaagatgagacagtttattactgtcagtcctacgattctagtttgtccggagtggttttcggtggtggtactaagctcaccgtgctg

SEQ ID NO: 1038 - DNA, 333 bp, Artificial Sequence
  Description of Artificial Sequence: Synthetic polynucleotide
agcagcgagctgacacaacctccctccgtttctggagccccaggtcaaagagtgactattagttgtgcaggctccctttcaaatataggagcaggttatgatgttcattggtatcaacaacttccaggcacagcaccaaaactccttatatacggaaacataaatcgcttgagcggtgtacccgaacgcttcagtggcagcaagtctggcaccagcgcttccttggcaataaccggactccaggctgaggatggcgctgattactattgccagtcctatgatagctccctctcttcatatgtcttcggaactggtacaaaagtgaccgtgctt

SEQ ID NO: 1039 - DNA, 330 bp, Artificial Sequence
  Description of Artificial Sequence: Synthetic polynucleotide
cagtccgtgttgacacaacctccaagcgcctcagggaccccaggacagggtgtgacaatatcctgttccggcagtagcagtaacattgggtctaataccgttaactggttccagcaactgcccggcacagctcccaaacttttgatctacagtgacaaccaacgaccctctggcgtacccgaccgtttctcaggttccaaaagcgggacctcagcatctctcgctatctccggtctgcaaagtgaggacgaagccgattactactgtgcagcctgggacgattcactgaatgggtatgttttcggaactggtaccaaagtgactgtgttg

SEQ ID NO: 1040 - DNA, 333 bp, Artificial Sequence
  Description of Artificial Sequence: Synthetic polynucleotide
cagtccgttctcacacagccaccatcagtaagtggcgctcccggtcaaagagtaactatttcatgtactggtagttcaagcaatataggtgctgactatgacgtccattggtatcagcacttgcccggcactgcacctaaacttctgatatacggaaatagtaacagacctagtggggttccagaccgtttctccgggagtaaaagtggaacaagcgcttcacttgcaattactggcctccaggctgaagatgaaaccgactattactgccaaagctacgattctagcctgtctgggtgggttttcggcgggggaactaaattgaccgtcttg

SEQ ID NO: 1041-1042 - skipped (2 entries)

SEQ ID NO: 1043 - DNA, 330 bp, Artificial Sequence
  Description of Artificial Sequence: Synthetic polynucleotide
caatctgtcctgactcaacctccctcagtctcagccgcaccaggacagaaggtgacaattagctgttcaggttcttcaagtaacatcggtaacaactacgtctcatggtatcaacagcttcccggaacagcacccaaactgctgatatatgataacaacaaacggccatctggaataccagaccggttctcaggctccaagagcggtactagcgcaactttgggaatcaccggtttgcagactggggatgaggcagactattactgcggcacctgggattccagtctgtctgcttatgtttttgggaccgggacaaaggtgactgtcctt

SEQ ID NO: 1044 - DNA, 330 bp, Artificial Sequence
  Description of Artificial Sequence: Synthetic polynucleotide
cagagtgtccttactcagcctcctagcgttagcgccgcccctggacagaaggttactatctcctgctcagggagttccagtaatattggaatcaattatgtgagttggtatcagcagttgcccggcaccgctcctaaattgcttatctatgacaacaataaacgacctagtggtatccctgatcgtttttctggatcaaaatctggtactagcgcaaccctcggtatcaccggactgcaaacaggtgatgaagcagactattattgcggcacctgggactcatcactctccgccgtcgttttcgggggcggaaccaaacttacagtattg

SEQ ID NO: 1045-1046 - skipped (2 entries)

SEQ ID NO: 1047 - DNA, 330 bp, Artificial Sequence
  Description of Artificial Sequence: Synthetic polynucleotide
caaagcgtgctgacacagcctccatcagtcagcgcagccccaggacagaaagtcactatttcttgcagcggtagcagctctaatatcggcaataattacgtctcctggtatcagcagctccccggcactgcacctaagttgctcatcttcgataataacaaacgtccttcaggcattcccgaccgattctcaggttcaaaatcaggaaccagtgctactttgggaataacaggtctgcaaactggtgatgaagctgattactactgcggtacctgggatagctctctcagtgcctatgtattcgggactggcacaaaggttactgttctg

SEQ ID NO: 1048 - DNA, 1353 bp, Artificial Sequence
  Description of Artificial Sequence: Synthetic polynucleotide
gaggtgcaactggtcgaatctggagggggactggttaagcctgggggtagcttgcgtcttagctgtgccgctagcggcttcaccttctcacgttacaatatgaattgggttcgacaggcccctggcaaaggtcttgagtgggtttcatccattaactcaaattcacgatatatctactacgcagattctgttaagggtcgattcactatatctcgagacagtgccaagaattccttgtacttgcagatgaatagcttgagggccgaagatactgccgtgtactactgtgcaaagacaatgggagattattattactattacggcatggacgtgtgggggcaggggacaacagtcaccgtaagcagtgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 1049 - skipped

SEQ ID NO: 1050 - DNA, 1362 bp, Artificial Sequence
  Description of Artificial Sequence: Synthetic polynucleotide
gaggtgcaattggttgagagtggcgggggggtcgttcaaccaggtcgcagcttgcgactctcttgtgccgccagcggtttcacattcataacttacgggatgcactgggtgcgacaggcacccggaaagggactggagtgggttgctgtggtgtctttcgatgagtctaataagtactatgcagactctgttaaaggccgatttactattagccgagacaactctaaaaatactctgtacctccagatgaatagtttgcgagcagaagatacagcagtttattattgtgccagggctcttagggacggtaataattgggattactttaacggcatggacgtatgggggcaaggcactaccgttactgtatcatccgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 1051 - skipped

SEQ ID NO: 1052 - DNA, 1359 bp, Artificial Sequence
  Description of Artificial Sequence: Synthetic polynucleotide
caagtgcagttggttgagtcaggtggaggagtggtccagcctggtcggagcctcagattgtcctgcgtggcaagcggattcaccttttcttcttacggtattcactgggtacggcaagcaccagggaaagggttggagtgggtcgctgtgatttggtacgacgggtcaaataagtactacgccgattccgtgaagggtcgctttacaatctctagggataactcaaaaaacacactgtacctgcaaatgaatagtctccgtgcagaagacacagcagtatattattctgtgcgcggagtagggcccacttcttattattacaactacggtatggatgtatggggccagggcactaccgttactgtatcttccgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 1053 - skipped

SEQ ID NO: 1054 - DNA, 1350 bp, Artificial Sequence
  Description of Artificial Sequence: Synthetic polynucleotide
gaggtacaacttgtggaaagtggaggcggtcttgtccaacctggaggatctctccgattgagttgcgcagccagcgggtttactttttcttcatacgccatgtcctgggtgcggcaagcaccaggtaaaggacttgagtgggtatctgctatttcaggggggataggctcaacatactatgctgatagcgtgaaaggtaggttcaccatttcccgtgacaatagtaaaaacacattgtggctccaaatgaacagccttagggctgaagacaccgctgtttactactgcgctaaagacggtgtaggggcaactccctattacttcgattattggggacaaggaaccttggtaacagtttcaagcgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 1055 - skipped

SEQ ID NO: 1056 - DNA, 1350 bp, Artificial Sequence
  Description of Artificial Sequence: Synthetic polynucleotide
gaagtccagttggtagaatctggaggcgggctggtacagcctggcggttccttgcgcctctcatgtgccgcaagcgggtttaccttcagctcttacgcaatgtcatgggtgcgtcaggcccctggaaaaggtctcgagtgggtcagtgccatttctgggggctccggctccacctactacgcagattcagttaaagggagatttacaatctcaagagataacagtaaaaacaccctctacctccagatgaactcacttcgagctgaggattcagcagtatattactgtgctaaagacggtgtaggtgcaactccctactatttcgactattggggccaagggactttggtgacagtaagtagtgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 1057 - skipped

SEQ ID NO: 1058 - DNA, 1350 bp, Artificial Sequence
  Description of Artificial Sequence: Synthetic polynucleotide
gaggtgcagcttgtagaatcagggggaggactggtgcagccaggcggatcattgagactgagttgtactgccagcggattcatattctccagttatgccatgagctgggtgcggcaggcacctggaaaagggcttgagtgggtctctgccataagcggtggatacggtgctccatactatgctgacaccgtaaaagggcggttcaccatctcccgagacaatagtaagaacaccctgtatctccagatgaactccctccgcgctgaagatacagccgtttattactgcgccaaagatggagtcggggccactccatactacttcgatgattggggacaagggatcctcgtcactgttagctcagcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 1059 - DNA, 1353 bp, Artificial Sequence
  Description of Artificial Sequence: Synthetic polynucleotide
gaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggcatcggctctacctactacgccgactctgtgaagggcagattcaccatcagccgggacaactccaagaacaccctgtggctgcagatgaactccctgagagccgaggataccgccgtgtactactgtgccaaagatggcgtgggcgctaccccttactacttcgattattggggccagggcaccctggtcaccgtttcttctgcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtccagatggcagcaggggaacgtcttctcatgctccgtgatgcatgaggctctgcacaaccactacacgcagaagtctctctccctgtctccgggaaaa

SEQ ID NO: 1060 - skipped

SEQ ID NO: 1061 - DNA, 1353 bp, Artificial Sequence
  Description of Artificial Sequence: Synthetic polynucleotide
gaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggatccggctctacctactacgccgactctgtgaagggcagattcaccatcagccgggacaactccaagaacaccctgtacctgcagatgaactccctgagagccgaggactccgccgtgtactactgtgctaaagatggcgtgggcgctaccccttactacttcgattattggggccagggcaccctggtcaccgtttcttctgcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtccagatggcagcaggggaacgtcttctcatgctccgtgatgcatgaggctctgcacaaccactacacgcagaagtctctctccctgtctccgggaaaa

SEQ ID NO: 1062 - skipped

SEQ ID NO: 1063 - DNA, 651 bp, Artificial Sequence
  Description of Artificial Sequence: Synthetic polynucleotide
caatccgtgcttacacaaccaccatcagtaagcggggctccaggccaaagagtaaccatctcatgcactggttccagcttcaaccttggtgccggatatgatgtccattggtatcaacaagtccctggaaccgtcccaaaactcctgatctacgataattcaaatcgaccttccggagtgccagatcggttcagtggatctaaaagcggcaccagtgcctctcttgccattactggattgcaagctgaagatgagacagtttattactgtcagtcctacgattctagtttgtccggagtggttttcggtggtggtactaagctcaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1064 - DNA, 651 bp, Artificial Sequence
  Description of Artificial Sequence: Synthetic polynucleotide
agcagcgagctgacacaacctccctccgtttctggagccccaggtcaaagagtgactattagttgtgcaggctccctttcaaatataggagcaggttatgatgttcattggtatcaacaacttccaggcacagcaccaaaactccttatatacggaaacataaatcgcttgagcggtgtacccgaacgcttcagtggcagcaagtctggcaccagcgcttccttggcaataaccggactccaggctgaggatggcgctgattactattgccagtcctatgatagctccctctcttcatatgtcttcggaactggtacaaaagtgaccgtgcttggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1065 - DNA, 648 bp, Artificial Sequence
  Description of Artificial Sequence: Synthetic polynucleotide
cagtccgtgttgacacaacctccaagcgcctcagggaccccaggacagggtgtgacaatatcctgttccggcagtagcagtaacattgggtctaataccgttaactggttccagcaactgcccggcacagctcccaaacttttgatctacagtgacaaccaacgaccctctggcgtacccgaccgtttctcaggttccaaaagcgggacctcagcatctctcgctatctccggtctgcaaagtgaggacgaagccgattactactgtgcagcctgggacgattcactgaatgggtatgttttcggaactggtaccaaagtgactgtgttgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1066 - DNA, 651 bp, Artificial Sequence
  Description of Artificial Sequence: Synthetic polynucleotide
cagtccgttctcacacagccaccatcagtaagtggcgctcccggtcaaagagtaactatttcatgtactggtagttcaagcaatataggtgctgactatgacgtccattggtatcagcacttgcccggcactgcacctaaacttctgatatacggaaatagtaacagacctagtggggttccagaccgtttctccgggagtaaaagtggaacaagcgcttcacttgcaattactggcctccaggctgaagatgaaaccgactattactgccaaagctacgattctagcctgtctgggtgggttttcggcgggggaactaaattgaccgtcttgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1067-1068 - skipped (2 entries)

SEQ ID NO: 1069 - DNA, 648 bp, Artificial Sequence
  Description of Artificial Sequence: Synthetic polynucleotide
caatctgtcctgactcaacctccctcagtctcagccgcaccaggacagaaggtgacaattagctgttcaggttcttcaagtaacatcggtaacaactacgtctcatggtatcaacagcttcccggaacagcacccaaactgctgatatatgataacaacaaacggccatctggaataccagaccggttctcaggctccaagagcggtactagcgcaactttgggaatcaccggtttgcagactggggatgaggcagactattactgcggcacctgggattccagtctgtctgcttatgtttttgggaccgggacaaaggtgactgtccttggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1070 - DNA, 648 bp, Artificial Sequence
  Description of Artificial Sequence: Synthetic polynucleotide
cagagtgtccttactcagcctcctagcgttagcgccgcccctggacagaaggttactatctcctgctcagggagttccagtaatattggaatcaattatgtgagttggtatcagcagttgcccggcaccgctcctaaattgcttatctatgacaacaataaacgacctagtggtatccctgatcgtttttctggatcaaaatctggtactagcgcaaccctcggtatcaccggactgcaaacaggtgatgaagcagactattattgcggcacctgggactcatcactctccgccgtcgttttcgggggcggaaccaaacttacagtattgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1071-1072 - skipped (2 entries)

SEQ ID NO: 1073 - DNA, 648 bp, Artificial Sequence
  Description of Artificial Sequence: Synthetic polynucleotide
caaagcgtgctgacacagcctccatcagtcagcgcagccccaggacagaaagtcactatttcttgcagcggtagcagctctaatatcggcaataattacgtctcctggtatcagcagctccccggcactgcacctaagttgctcatcttcgataataacaaacgtccttcaggcattcccgaccgattctcaggttcaaaatcaggaaccagtgctactttgggaataacaggtctgcaaactggtgatgaagctgattactactgcggtacctgggatagctctctcagtgcctatgtattcgggactggcacaaaggttactgttctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1074-1133 - skipped (60 entries)

SEQ ID NO: 1134 - protein, 483 aa, Artificial Sequence
  Description of Artificial Sequence: Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVLGGSEGKSSGSGSESKSTGGSEVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 1135 - DNA, 1449 bp, Artificial Sequence
  Description of Artificial Sequence: Synthetic polynucleotide
cagtctgtgctgacccagcctccttctgtgtctgctgctcctggccagaaagtgaccatctcctgctccggctcctcctccaacatcggcaacaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttccctgtccgcttatgtgtttggcaccggcaccaaagtgaccgtgttgggcggctccgagggcaagagcagcggcagcggcagcgagagcaagagcaccggcggcagcgaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggcatcggctctacctactacgccgactctgtgaagggcagattcaccatcagccgggacaactccaagaacaccctgtggctgcagatgaactccctgagagccgaggataccgccgtgtactactgtgccaaagatggcgtgggcgctaccccttactacttcgattattggggccagggcaccctggtcaccgtttcttctgagcccaaatctagcgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 1136 - protein, 483 aa, Artificial Sequence
  Description of Artificial Sequence: Synthetic polypeptide
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGGSEGKSSGSGSESKSTGGSEVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 1137 - DNA, 1449 bp, Artificial Sequence
  Description of Artificial Sequence: Synthetic polynucleotide
cagtctgtgctgacccagcctccttctgtgtctgctgctcctggccagaaagtgaccatctcctgctccggctcctcctccaacatcggcatcaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttctctgtccgccgttgtttttggcggtggcaccaagctgacagtgctcggcggctccgagggcaagagcagcggcagcggcagcgagagcaagagcaccggcggcagcgaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggcatcggctctacctactacgccgactctgtgaagggcagattcaccatcagccgggacaactccaagaacaccctgtggctgcagatgaactccctgagagccgaggataccgccgtgtactactgtgccaaagatggcgtgggcgctaccccttactacttcgattattggggccagggcaccctggtcaccgtttcttctgagcccaaatctagcgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 1138 - protein, 483 aa, Artificial Sequence
  Description of Artificial Sequence: Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVLGGSEGKSSGSGSESKSTGGSEVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 1139 - DNA, 1449 bp, Artificial Sequence
  Description of Artificial Sequence: Synthetic polynucleotide
cagtctgtgctgacccagcctccttctgtgtctgctgctcctggccagaaagtgaccatctcctgctccggctcctcctccaacatcggcaacaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttccctgtccgcttatgtgtttggcaccggcaccaaagtgaccgtgttgggcggctccgagggcaagagcagcggcagcggcagcgagagcaagagcaccggcggcagcgaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggatccggctctacctactacgccgactctgtgaagggcagattcaccatcagccgggacaactccaagaacaccctgtacctgcagatgaactccctgagagccgaggactccgccgtgtactactgtgctaaagatggcgtgggcgctaccccttactacttcgattattggggccagggcaccctggtcaccgtttcttctgagcccaaatctagcgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 1140 - protein, 483 aa, Artificial Sequence
  Description of Artificial Sequence: Synthetic polypeptide
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGGSEGKSSGSGSESKSTGGSEVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 1141 - DNA, 1449 bp, Artificial Sequence
  Description of Artificial Sequence: Synthetic polynucleotide
cagtctgtgctgacccagcctccttctgtgtctgctgctcctggccagaaagtgaccatctcctgctccggctcctcctccaacatcggcatcaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttctctgtccgccgttgtttttggtggaggcaccaagctgacagtgctcggcggctccgagggcaagagcagcggcagcggcagcgagagcaagagcaccggcggcagcgaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggatccggctctacctactacgccgactctgtgaagggcagattcaccatcagccgggacaactccaagaacaccctgtacctgcagatgaactccctgagagccgaggactccgccgtgtactactgtgctaaagatggcgtgggcgctaccccttactacttcgattattggggccagggcaccctggtcaccgtttcttctgagcccaaatctagcgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 1142 - protein, 432 aa, Artificial Sequence
  Description of Artificial Sequence: Synthetic polypeptide
FKIPIEELEDRVFVNCNTSITWVEGTVGTLLSDITRLDLGKRILDPRGIYRCNGTDIYKDKESTVQVHYRMGSADDAKKDAAKKDDAKKDDAKKDGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGLNDIFEAQKIEWHE

SEQ ID NO: 1143 - protein, 446 aa, Artificial Sequence
  Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1144 - protein, 214 aa, Artificial Sequence
  Description of Artificial Sequence: Synthetic polypeptide
DILLTQSPGILSVSPGERVSFSCRARQSIGTAIHWYQQRTNGSPRLLIKYASESISGIPSRFSGSGSGTDFTLTINSVESEDIADYYCQQSNSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1145-1148 - skipped (4 entries)

SEQ ID NO: 1149 - DNA, 1350 bp, Artificial Sequence
  Description of Artificial Sequence: Synthetic polynucleotide
caggtgcagctgcagcagtctggccctagactcgtgcggccttcccagaccctgtctctgacctgtgccatctccggcgactccgtgttcaacaacaacgccgcctggtcctggatccggcagtctccatctcgcggtctggagtggctcggtcgcacctactaccgctctaaatggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccaaggcaccctcgtgaccgtgtcctctgcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctgctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 1150 - DNA, 648 bp, Artificial Sequence
  Description of Artificial Sequence: Synthetic polynucleotide
cagtctgctctgacccagcctgcctccgtgtctggctctcccggccagtccatcaccatcagctgtaccggcacctcctccaacatcggcacctacaagttcgtgtcctggtatcagcaacaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgactgtcctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1151 - DNA, 360 bp, Artificial Sequence
  Description of Artificial Sequence: Synthetic polynucleotide
caggtgcagctgcagcagtctggccctagactcgtgcggccttcccagaccctgtctctgacctgtgccatctccggcgactccgtgttcaacaacaacgccgcctggtcctggatccggcagagcccttctagaggcctggaatggctgggccggacctactaccggtccaagtggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccagggcaccctcgtgaccgtgtcctct

SEQ ID NO: 1152 - DNA, 330 bp, Artificial Sequence
  Description of Artificial Sequence: Synthetic polynucleotide
cagtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctg

SEQ ID NO: 1153 - DNA, 360 bp, Artificial Sequence
  Description of Artificial Sequence: Synthetic polynucleotide
caggttcagctgcagcagtctggccctggactggtcaagccctctcagaccctgtctctgacctgtgccatctccggcgactccgtgttcaacaacaatgccgcctggtcctggattcggcagtctcctagtagaggcctggaatggctgggcagaacctactaccggtccaagtggctgtacgactacgccgtgtccgtgaagtcccggatcaccatcaatcccgacacctccaagaaccagttctccctgcagctcaacagcgtgacccctgaggataccgccgtgtactactgtgccagaggctactcctcctccttcgactattggggccagggcacactggtcaccgtttcttct

SEQ ID NO: 1154 - DNA, 330 bp, Artificial Sequence
  Description of Artificial Sequence: Synthetic polynucleotide
cagtctgctctgacccagcctgcttctgtgtctggctctcccggccagtccatcaccatctcttgtaccggcacctcctccaacatcggcacctacaagttcgtgtcctggtatcagcagcaccccggcaaggcccctaaagtgatgatctacgaggtgtccaagcggccctctggcgtgtccaacagattctccggctccaagtccggcaataccgcctctctgacaatcagcggactgcaggctgaggacgaggccgactactactgtgtgtcttacgctggctctggcaccctgttgtttggcggcggaacaaagctgactgtgctg

SEQ ID NO: 1155 - DNA, 360 bp, Artificial Sequence
  Description of Artificial Sequence: Synthetic polynucleotide
caggtgcagctgcagcagagcggccccggcctggtcaagcccagccagaccctgagcctgacctgcgccatcagcggcgacagcgtgttcaacaacaacgccgcctggtcctggatccgccagagccccagccgcggcctggagtggctgggccgcacctactaccgcagcaagtggctgtacgactacgccgtgtccgtgaagtcccgcatcaccatcaaccccgacaccagcaagaaccagttctccctgcagctgaacagcgtgacccccgaggacaccgccgtgtactactgcgcccgcggctacagcagcagcttcgactactggggccagggcaccctggtcaccgtgtccagc

SEQ ID NO: 1156 - DNA, 330 bp, Artificial Sequence
  Description of Artificial Sequence: Synthetic polynucleotide
cagtctgctctgacccagcctgcctccgtgtctggctctcccggccagtccatcaccatcagctgtaccggcacctcctccaacatcggcacctacaagttcgtgtcctggtatcagcagcaccccggcaaggcccccaaagtgatgatctacgaggtgtccaagcggccctccggcgtgtccaacagattctccggctccaagtccggcaacaccgcctccctgacaatcagcggactgcaggccgaggacgaggccgactactactgtgtgtcctacgccggctctggcaccctgctgtttggcggcggaacaaagctgaccgtgctg

SEQ ID NO: 1157 - DNA, 348 bp, Artificial Sequence
  Description of Artificial Sequence: Synthetic polynucleotide
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctca

SEQ ID NO: 1158 - DNA, 321 bp, Artificial Sequence
  Description of Artificial Sequence: Synthetic polynucleotide
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaag

SEQ ID NO: 1159-1166 - skipped (8 entries)

SEQ ID NO: 1167 - protein, 444 aa, Artificial Sequence
  Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSP

SEQ ID NO: 1168-1169 - skipped (2 entries)

SEQ ID NO: 1170 - DNA, 330 bp, Artificial Sequence
  Description of Artificial Sequence: Synthetic polynucleotide
cagtctgctctgacccagcctgcctccgtgtctggctctcccggccagtccatcaccatcagctgtaccggcacctcctccaacatcggcacctacaagttcgtgtcctggtatcagcaacaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgactgtcctg

SEQ ID NO: 1171 - DNA, 1347 bp, Artificial Sequence
  Description of Artificial Sequence: Synthetic polynucleotide
caggtgcagctgcagcagtctggccctagactcgtgcggccttcccagaccctgtctctgacctgtgccatctccggcgactccgtgttcaacaacaacgccgcctggtcctggatccggcagagcccttctagaggcctggaatggctgggccggacctactaccggtccaagtggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccagggcaccctcgtgaccgtgtcctctgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctgctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 1172 - DNA, 648 bp, Artificial Sequence
  Description of Artificial Sequence: Synthetic polynucleotide
cagtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1173 - DNA, 1350 bp, Artificial Sequence
  Description of Artificial Sequence: Synthetic polynucleotide
caggttcagctgcagcagtctggccctggactggtcaagccctctcagaccctgtctctgacctgtgccatctccggcgactccgtgttcaacaacaatgccgcctggtcctggattcggcagtctcctagtagaggcctggaatggctgggcagaacctactaccggtccaagtggctgtacgactacgccgtgtccgtgaagtcccggatcaccatcaatcccgacacctccaagaaccagttctccctgcagctcaacagcgtgacccctgaggataccgccgtgtactactgtgccagaggctactcctcctccttcgactattggggccagggcacactggtcaccgtttcttctgcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacttgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctgctctacagcaagctcaccgtggacaagtccagatggcagcaggggaacgtcttctcatgctccgtgatgcatgaggctctgcacaaccactacacgcagaagtctctctccctgtctccgggaaaa

SEQ ID NO: 1174 - DNA, 648 bp, Artificial Sequence
  Description of Artificial Sequence: Synthetic polynucleotide
cagtctgctctgacccagcctgcttctgtgtctggctctcccggccagtccatcaccatctcttgtaccggcacctcctccaacatcggcacctacaagttcgtgtcctggtatcagcagcaccccggcaaggcccctaaagtgatgatctacgaggtgtccaagcggccctctggcgtgtccaacagattctccggctccaagtccggcaataccgcctctctgacaatcagcggactgcaggctgaggacgaggccgactactactgtgtgtcttacgctggctctggcaccctgttgtttggcggcggaacaaagctgactgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1175 - DNA, 1347 bp, Artificial Sequence
  Description of Artificial Sequence: Synthetic polynucleotide
caggtgcagctgcagcagagcggccccggcctggtcaagcccagccagaccctgagcctgacctgcgccatcagcggcgacagcgtgttcaacaacaacgccgcctggtcctggatccgccagagccccagccgcggcctggagtggctgggccgcacctactaccgcagcaagtggctgtacgactacgccgtgtccgtgaagtcccgcatcaccatcaaccccgacaccagcaagaaccagttctccctgcagctgaacagcgtgacccccgaggacaccgccgtgtactactgcgcccgcggctacagcagcagcttcgactactggggccagggcaccctggtcaccgtgtccagcgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctgctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 1176 - DNA, 648 bp, Artificial Sequence
  Description of Artificial Sequence: Synthetic polynucleotide
cagtctgctctgacccagcctgcctccgtgtctggctctcccggccagtccatcaccatcagctgtaccggcacctcctccaacatcggcacctacaagttcgtgtcctggtatcagcagcaccccggcaaggcccccaaagtgatgatctacgaggtgtccaagcggccctccggcgtgtccaacagattctccggctccaagtccggcaacaccgcctccctgacaatcagcggactgcaggccgaggacgaggccgactactactgtgtgtcctacgccggctctggcaccctgctgtttggcggcggaacaaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 1177 - DNA, 1335 bp, Artificial Sequence
  Description of Artificial Sequence: Synthetic polynucleotide
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcctgctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 1178 - DNA, 642 bp, Artificial Sequence
  Description of Artificial Sequence: Synthetic polynucleotide
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 1179-1185 - skipped (7 entries)

SEQ ID NO: 1186 - protein, 243 aa, Artificial Sequence
  Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 1187 - protein, 243 aa, Artificial Sequence
  Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSGGSEGKSSGSGSESKSTGGSDIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 1188-1191 - skipped (4 entries)

SEQ ID NO: 1192 - protein, 475 aa, Artificial Sequence
  Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1193 - skipped

SEQ ID NO: 1194 - protein, 449 aa, Artificial Sequence
  Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPG

SEQ ID NO: 1195 - protein, 217 aa, Artificial Sequence
  Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1196-1217 - skipped (22 entries)

SEQ ID NO: 1218 - protein, 449 aa, Artificial Sequence
  Description of Artificial Sequence: Synthetic polypeptide
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNRFTQKSLSLSPG

SEQ ID NO: 1219 - protein, 218 aa, Artificial Sequence
  Description of Artificial Sequence: Synthetic polypeptide
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1220-1237 - skipped (18 entries)

SEQ ID NO: 1238 - protein, 449 aa, Artificial Sequence
  Description of Artificial Sequence: Synthetic polypeptide
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 1239-1241 - skipped (3 entries)

SEQ ID NO: 1242 - protein, 451 aa, Artificial Sequence
  Description of Artificial Sequence: Synthetic polypeptide
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 1243 - skipped

SEQ ID NO: 1244 - protein, 451 aa, Artificial Sequence
  Description of Artificial Sequence: Synthetic polypeptide
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDAVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 1245-1247 - skipped (3 entries)

SEQ ID NO: 1248 - protein, 478 aa, Artificial Sequence
  Description of Artificial Sequence: Synthetic polypeptide
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVLGGSEGKSSGSGSESKSTGGSQVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1249 - skipped

SEQ ID NO: 1250 - protein, 487 aa, Artificial Sequence
  Description of Artificial Sequence: Synthetic polypeptide
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVLGGSEGKSSGSGSESKSTGGSEVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCAREGVGADYGDYYYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1251 - skipped

SEQ ID NO: 1252 - protein, 481 aa, Artificial Sequence
  Description of Artificial Sequence: Synthetic polypeptide
EIVLTQSPGTLSVSPGERATLSCRASQSVRSNLAWYQQKPGQAPRLLIYGASTRATGIPARFSGSGSGTEFTLTISSLQSEDFAVYYCHQYNDWPPYTFGQGTKLEIKGGSEGKSSGSGSESKSTGGSQVQLQESGGGVVQPGRSLRLSCAASGFTFSTYGMHWVRQAPGKGLEWVAFISYDGSNKYYADSVKGRFTISRDNSKHTLYLQMNSLRAEDTAVYYCAGRDNLRFLEWFMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1253 - skipped

SEQ ID NO: 1254 - protein, 481 aa, Artificial Sequence
  Description of Artificial Sequence: Synthetic polypeptide
SYELTQPPSVSVAPGQTARITCGGNNIGSKSVHWYQQKPGQAPVLVVYDDSDRPSGIPERFSGSNSGNTATLTISRVEAGDEADYYCQVWDSSTDHVVFGGGTKLTVLGGSEGKSSGSGSESKSTGGSEVQLVESGGGVVQPGRSLRLSCAASGFTFSSYGMNWVRQAPGKGLEWVAVTSYDGSNKYYADSVKGRFTISRDISKNTLYLQMSSLRAEDTAVYYCARDPYSSSWNGAFDIWGPGTMVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1255 - skipped

SEQ ID NO: 1256 - protein, 486 aa, Artificial Sequence
  Description of Artificial Sequence: Synthetic polypeptide
QSVLTQPPSASGTPGQGVTISCSGSSSNIGSNTVNWFQQLPGTAPKLLIYSDNQRPSGVPDRFSGSKSGTSASLAISGLQSEDEADYYCAAWDDSLNGYVFGTGTKVTVLGGSEGKSSGSGSESKSTGGSEVQLVESGGGVVQPGRSLRLSCAASGFTFITYGMHWVRQAPGKGLEWVAVVSFDESNKYYADSVKGRFTISRDNSKNTLYLQMNSLRAEDTAVYYCARALRDGNNWDYFNGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1257 - skipped

SEQ ID NO: 1258 - protein, 487 aa, Artificial Sequence
  Description of Artificial Sequence: Synthetic polypeptide
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVLGGSEGKSSGSGSESKSTGGSEVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCARDGVGADYGDYYYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1259 - skipped

SEQ ID NO: 1260 - protein, 481 aa, Artificial Sequence
  Description of Artificial Sequence: Synthetic polypeptide
QSVLTQPPSVSVAPGQTARITCGGNNSGSKSVHWYQQKPGQAPVLVVYDDSDRPSGIPERFSGSNSGNTATLTISRVEAGDEADYYCQVWDSSSDHGVFGGGTKLTVLGGSEGKSSGSGSESKSTGGSQVQLVESGGGEVQPGRSLRLSCAASGFSFSGYGMHWVRQAPGKGLEWVAVMSYDGSNRFYVDSVRGRFSISRDNSKNTLYLQMNSLRPEDTAVYYCARDTVWGSHPDAFDIWGQGTVVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1261 - skipped

SEQ ID NO: 1262 - protein, 478 aa, Artificial Sequence
  Description of Artificial Sequence: Synthetic polypeptide
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVLGGSEGKSSGSGSESKSTGGSQVQLVESGGGVVQPGRSLRLSCAASGFTFSSYNMNWVRQAPGKGLEWVAIIYYDGSNKYYADSVKGRFTISRDISKNTLYLQMNSLRAEDTAVYYCARERGRDYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1263 - skipped

SEQ ID NO: 1264 - protein, 479 aa, Artificial Sequence
  Description of Artificial Sequence: Synthetic polypeptide
QSVLTQPPSASGTPGQRVTISCSGSSSNIGSNTVNWYQQLPGTAPKLLIYSSNQRPSGVPDRFSGSKSGTSASLAISGLQSEDEADYYCAAWDDSLNGVVFGGGTKLTVLGGSEGKSSGSGSESKSTGGSEVQLLESGPGLVKPSETLSLTCTVSGGSIISYYWSWIRQPAGKGLEWIGRIYSSGSTNYNPSLKSRVTMSVDTSKNQFSLKLSSVTAADTAVYYCAKVGVWPGAFDIWGQGTMVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1265 - skipped

SEQ ID NO: 1266 - protein, 485 aa, Artificial Sequence
  Description of Artificial Sequence: Synthetic polypeptide
EVQLVESGGGVVQPGRSLRLSCAASGFSFSGYGMHWVRQAPGKGLEWVAVISYDGSNKYYADSVKGRFTISRDNSKNTLYLQMNSLRVEDTAVYYCARDRIWGSRGYYYGMDVWGQGTTVTVSSGGSEGKSSGSGSESKSTGGSQSALTQPASVSGSPGQSITISCTGASSDVGGYNYVSWYQQHPGKAPKLMIYEVSNRPSGVSNRFSGSKSGNTASLTISGLQAEDEADYYCSSYTITSTLVFGGGTKLTVLEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1267 - skipped

SEQ ID NO: 1268 - protein, 481 aa, Artificial Sequence
  Description of Artificial Sequence: Synthetic polypeptide
QVQLVESGGGEVQPGRSLRLSCAASGFSFSGYGMHWVRQAPGKGLEWVAVMSYDGSNRFYVDSVRGRFSISRDNSKNTLYLQMNSLRPEDTAVYYCARDTVWGSHPDAFDIWGQGTVVTVSSGGSEGKSSGSGSESKSTGGSQSVLTQPPSVSVAPGQTARITCGGNNSGSKSVHWYQQKPGQAPVLVVYDDSDRPSGIPERFSGSNSGNTATLTISRVEAGDEADYYCQVWDSSSDHGVFGGGTKLTVLEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1269 - skipped

SEQ ID NO: 1270 - protein, 481 aa, Artificial Sequence
  Description of Artificial Sequence: Synthetic polypeptide
QVQLQESGGDVVQPGRSLRLSCAASGFSFSGYGLHWVRQAPGRGLEWVTLISYDGSNKYYADSVKGRFTISRDNSKNTLYLQMNSLRAEDTAVYYCAKTTVSDPYYYGMDVWGQGTTVTVSSGGSEGKSSGSGSESKSTGGSSYELTQPPSVSVAPGQTARITCGGNNIGSKSVHWYQQKPGQAPVLVVYDDSDRPSGIPERFSGTNSGNTATLTISRAEAGDEADYYCQVWDSSSDHVVFGGGTKLTVLEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 1271-1403 - skipped (133 entries)

SEQ ID NO: 1404 - protein, 451 aa, Artificial Sequence
  Description of Artificial Sequence: Synthetic polypeptide
EVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 1405-1415 - skipped (11 entries)

SEQ ID NO: 1416 - protein, 750 aa, Pan troglodytes
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVLDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRHGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYNISMKHPQEMKTYSVSFDSLFSAVKNFTEIASKFTERLQDFDKSNPILLRMMNDQLMFLERAFIDPLGLPDRPFYRHVIYAPSSHNKYAGESFPGIYDALFDIESKVDPSKAWGDVKRQISVAAFTVQAAAETLSEVA

SEQ ID NO: 1417 - protein, 750 aa, Macaca fascicularis
MWNLLHETDSAVATARRPRWLCAGALVLAGGFFLLGFLFGWFIKSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPDDYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGMLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 1418 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 1419 - protein, 20 aa, Artificial Sequence
  Description of Artificial Sequence: Synthetic peptide
GGSEGKSSGSGSESKSTGGS

SEQ ID NO: 1420 - protein, 8 aa, Artificial Sequence
  Description of Artificial Sequence: Synthetic peptide
GGGSGGGS

SEQ ID NO: 1421 - protein, 12 aa, Artificial Sequence
  Description of Artificial Sequence: Synthetic peptide
GGGSGGGSGGGS

SEQ ID NO: 1422 - protein, 16 aa, Artificial Sequence
  Description of Artificial Sequence: Synthetic peptide
GGGSGGGSGGGSGGGS

SEQ ID NO: 1423 - protein, 20 aa, Artificial Sequence
  Description of Artificial Sequence: Synthetic peptide
GGGSGGGSGGGSGGGSGGGS

SEQ ID NO: 1424 - protein, 15 aa, Artificial Sequence
  Description of Artificial Sequence: Synthetic peptide
GGGGSGGGGSGGGGS

SEQ ID NO: 1425 - protein, 20 aa, Artificial Sequence
  Description of Artificial Sequence: Synthetic peptide
GGGGSGGGGSGGGGSGGGGS

SEQ ID NO: 1426 - protein, 25 aa, Artificial Sequence
  Description of Artificial Sequence: Synthetic peptide
GGGGSGGGGSGGGGSGGGGSGGGGS

SEQ ID NO: 1427 - protein, 18 aa, Artificial Sequence
  Description of Artificial Sequence: Synthetic peptide
GSTSGSGKPGSGEGSTKG

SEQ ID NO: 1428 - protein, 14 aa, Artificial Sequence
  Description of Artificial Sequence: Synthetic peptide
IRPRAIGGSKPRVA

SEQ ID NO: 1429 - protein, 15 aa, Artificial Sequence
  Description of Artificial Sequence: Synthetic peptide
GKGGSGKGGSGKGGS

SEQ ID NO: 1430 - protein, 15 aa, Artificial Sequence
  Description of Artificial Sequence: Synthetic peptide
GGKGSGGKGSGGKGS

SEQ ID NO: 1431 - protein, 15 aa, Artificial Sequence
  Description of Artificial Sequence: Synthetic peptide
GGGKSGGGKSGGGKS

SEQ ID NO: 1432 - protein, 15 aa, Artificial Sequence
  Description of Artificial Sequence: Synthetic peptide
GKGKSGKGKSGKGKS

SEQ ID NO: 1433 - protein, 15 aa, Artificial Sequence
  Description of Artificial Sequence: Synthetic peptide
GGGKSGGKGSGKGGS

SEQ ID NO: 1434 - protein, 15 aa, Artificial Sequence
  Description of Artificial Sequence: Synthetic peptide
GKPGSGKPGSGKPGS

SEQ ID NO: 1435 - protein, 20 aa, Artificial Sequence
  Description of Artificial Sequence: Synthetic peptide
GKPGSGKPGSGKPGSGKPGS

SEQ ID NO: 1436 - protein, 20 aa, Artificial Sequence
  Description of Artificial Sequence: Synthetic peptide
GKGKSGKGKSGKGKSGKGKS

SEQ ID NO: 1437 - protein, 14 aa, Artificial Sequence
  Description of Artificial Sequence: Synthetic peptide
STAGDTHLGGEDFD

SEQ ID NO: 1438 - protein, 15 aa, Artificial Sequence
  Description of Artificial Sequence: Synthetic peptide
GEGGSGEGGSGEGGS

SEQ ID NO: 1439 - protein, 15 aa, Artificial Sequence
  Description of Artificial Sequence: Synthetic peptide
GGEGSGGEGSGGEGS

SEQ ID NO: 1440 - protein, 15 aa, Artificial Sequence
  Description of Artificial Sequence: Synthetic peptide
GEGESGEGESGEGES

SEQ ID NO: 1441 - protein, 15 aa, Artificial Sequence
  Description of Artificial Sequence: Synthetic peptide
GGGESGGEGSGEGGS

SEQ ID NO: 1442 - protein, 20 aa, Artificial Sequence
  Description of Artificial Sequence: Synthetic peptide
GEGESGEGESGEGESGEGES

SEQ ID NO: 1443 - skipped

SEQ ID NO: 1444 - protein, 19 aa, Artificial Sequence
  Description of Artificial Sequence: Synthetic peptide
PRGASKSGSASQTGSAPGS

SEQ ID NO: 1445 - DNA, 19 bp, Artificial Sequence
  Description of Artificial Sequence: Synthetic oligonucleotide
gtaaagagaaggaaagaag

SEQ ID NO: 1446 - protein, 19 aa, Artificial Sequence
  Description of Artificial Sequence: Synthetic peptide
GTSGSSGSGSGGSGSGGGG

SEQ ID NO: 1447 - skipped

SEQ ID NO: 1448 - protein, 4 aa, Artificial Sequence
  Description of Artificial Sequence: Synthetic peptide
GSGS

SEQ ID NO: 1449 - protein, 10 aa, Artificial Sequence
  Description of Artificial Sequence: Synthetic peptide
APAPAPAPAP

SEQ ID NO: 1450 - protein, 20 aa, Artificial Sequence
  Description of Artificial Sequence: Synthetic peptide
APAPAPAPAPAPAPAPAPAP

SEQ ID NO: 1451 - protein, 32 aa, Artificial Sequence
  Description of Artificial Sequence: Synthetic polypeptide
AEAAAKEAAAKEAAAAKEAAAAKEAAAAKAAA

SEQ ID NO: 1452 - protein, 16 aa, Artificial Sequence
  Description of Artificial Sequence: Synthetic peptide
GTEGKSSGSGSESKST

SEQ ID NO: 1453 - DNA, 360 bp, Artificial Sequence
  Description of Artificial Sequence: Synthetic polynucleotide
caggtgcagctgcagcagtctggccctagactcgtgcggccttcccagaccctgtctctgacctgtgccatctccggcgactccgtgttcaacaacaacgccgcctggtcctggatccggcagtctccatctcgcggtctggagtggctcggtcgcacctactaccgctctaaatggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccaaggcaccctcgtgaccgtgtcctct

SEQ ID NO: 1454 - skipped

SEQ ID NO: 1455 - protein, 479 aa, Artificial Sequence
  Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1456-1462 - skipped (7 entries)

SEQ ID NO: 1463 - protein, 121 aa, Artificial Sequence
  Description of Artificial Sequence: Synthetic polypeptide
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYNGFPYWGQGTLVTVSS

SEQ ID NO: 1464 - protein, 106 aa, Artificial Sequence
  Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 1465-1466 - skipped (2 entries)

SEQ ID NO: 1467 - protein, 5 aa, Artificial Sequence
  Description of Artificial Sequence: Synthetic peptide
RSTMH

SEQ ID NO: 1468 - protein, 17 aa, Artificial Sequence
  Description of Artificial Sequence: Synthetic peptide
YINPSSAYTNYNQKFQG

SEQ ID NO: 1469 - protein, 12 aa, Artificial Sequence
  Description of Artificial Sequence: Synthetic peptide
PQVHYDYNGFPY

SEQ ID NO: 1470 - protein, 10 aa, Artificial Sequence
  Description of Artificial Sequence: Synthetic peptide
SASSSVSYMN

SEQ ID NO: 1471 - protein, 7 aa, Artificial Sequence
  Description of Artificial Sequence: Synthetic peptide
DSSKLAS

SEQ ID NO: 1472 - protein, 9 aa, Artificial Sequence
  Description of Artificial Sequence: Synthetic peptide
QQWSRNPPT

SEQ ID NO: 1473 - protein, 7 aa, Artificial Sequence
  Description of Artificial Sequence: Synthetic peptide
GYTFTRS

SEQ ID NO: 1474 - protein, 6 aa, Artificial Sequence
  Description of Artificial Sequence: Synthetic peptide
NPSSAY

SEQ ID NO: 1475 - protein, 11 aa, Artificial Sequence
  Description of Artificial Sequence: Synthetic peptide
PQVHYDYNGFP

SEQ ID NO: 1476 - protein, 6 aa, Artificial Sequence
  Description of Artificial Sequence: Synthetic peptide
SSSVSY

SEQ ID NO: 1477 - protein, 3 aa, Artificial Sequence
  Description of Artificial Sequence: Synthetic peptide
DSS

SEQ ID NO: 1478 - protein, 6 aa, Artificial Sequence
  Description of Artificial Sequence: Synthetic peptide
WSRNPP

SEQ ID NO: 1479 - protein, 8 aa, Artificial Sequence
  Description of Artificial Sequence: Synthetic peptide
GYTFTRST

SEQ ID NO: 1480 - protein, 8 aa, Artificial Sequence
  Description of Artificial Sequence: Synthetic peptide
INPSSAYT

SEQ ID NO: 1481 - protein, 14 aa, Artificial Sequence
  Description of Artificial Sequence: Synthetic peptide
ASPQVHYDYNGFPY

SEQ ID NO: 1482 - protein, 5 aa, Artificial Sequence
  Description of Artificial Sequence: Synthetic peptide
SSVSY

SEQ ID NO: 1483 - protein, 713 aa, Artificial Sequence
  Description of Artificial Sequence: Synthetic polypeptide
HHHHHHKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNKTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 1484 - protein, 6 aa, Artificial Sequence
  Description of Artificial Sequence: Synthetic 6xHis tag
HHHHHH

SEQ ID NO: 1485 - protein, 251 aa, Artificial Sequence
  Description of Artificial Sequence: Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVLGGSEGKSSGSGSESKSTGGSEVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSS

SEQ ID NO: 1486 - protein, 251 aa, Artificial Sequence
  Description of Artificial Sequence: Synthetic polypeptide
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGGSEGKSSGSGSESKSTGGSEVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSS

SEQ ID NO: 1487 - protein, 251 aa, Artificial Sequence
  Description of Artificial Sequence: Synthetic polypeptide
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVLGGSEGKSSGSGSESKSTGGSEVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSS

SEQ ID NO: 1488 - protein, 251 aa, Artificial Sequence
  Description of Artificial Sequence: Synthetic polypeptide
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGGSEGKSSGSGSESKSTGGSEVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSS

SEQ ID NO: 1489 - protein, 250 aa, Artificial Sequence
  Description of Artificial Sequence: Synthetic polypeptide
EIVLTQSPGTLSVSPGERATLSCRASQSVRSNLAWYQQKPGQAPRLLIYGASTRATGIPARFSGSGSGTEFTLTISSLQSEDFAVYYCHQYNDWPPYTFGQGTKLEIKGGSEGKSSGSGSESKSTGGSQVQLQESGGGVVQPGRSLRLSCAASGFTFSTYGMHWVRQAPGKGLEWVAFISYDGSNKYYADSVKGRFTISRDNSKHTLYLQMNSLRAEDTAVYYCAGRDNLRFLEWFMDVWGQGTTVTVSS

SEQ ID NO: 1490 - protein, 248 aa, Artificial Sequence
  Description of Artificial Sequence: Synthetic polypeptide
QSVLTQPPSASGTPGQRVTISCSGSSSNIGSNTVNWYQQLPGTAPKLLIYSSNQRPSGVPDRFSGSKSGTSASLAISGLQSEDEADYYCAAWDDSLNGVVFGGGTKLTVLGGSEGKSSGSGSESKSTGGSEVQLLESGPGLVKPSETLSLTCTVSGGSIISYYWSWIRQPAGKGLEWIGRIYSSGSTNYNPSLKSRVTMSVDTSKNQFSLKLSSVTAADTAVYYCAKVGVWPGAFDIWGQGTMVTVSS

SEQ ID NO: 1491 - protein, 247 aa, Artificial Sequence
  Description of Artificial Sequence: Synthetic polypeptide
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVLGGSEGKSSGSGSESKSTGGSQVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSS

SEQ ID NO: 1492 - protein, 256 aa, Artificial Sequence
  Description of Artificial Sequence: Synthetic polypeptide
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVLGGSEGKSSGSGSESKSTGGSEVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCAREGVGADYGDYYYYGMDVWGQGTTVTVSS

SEQ ID NO: 1493 - protein, 250 aa, Artificial Sequence
  Description of Artificial Sequence: Synthetic polypeptide
SYELTQPPSVSVAPGQTARITCGGNNIGSKSVHWYQQKPGQAPVLVVYDDSDRPSGIPERFSGSNSGNTATLTISRVEAGDEADYYCQVWDSSTDHVVFGGGTKLTVLGGSEGKSSGSGSESKSTGGSEVQLVESGGGVVQPGRSLRLSCAASGFTFSSYGMNWVRQAPGKGLEWVAVTSYDGSNKYYADSVKGRFTISRDISKNTLYLQMSSLRAEDTAVYYCARDPYSSSWNGAFDIWGPGTMVTVSS

SEQ ID NO: 1494 - protein, 255 aa, Artificial Sequence
  Description of Artificial Sequence: Synthetic polypeptide
QSVLTQPPSASGTPGQGVTISCSGSSSNIGSNTVNWFQQLPGTAPKLLIYSDNQRPSGVPDRFSGSKSGTSASLAISGLQSEDEADYYCAAWDDSLNGYVFGTGTKVTVLGGSEGKSSGSGSESKSTGGSEVQLVESGGGVVQPGRSLRLSCAASGFTFITYGMHWVRQAPGKGLEWVAVVSFDESNKYYADSVKGRFTISRDNSKNTLYLQMNSLRAEDTAVYYCARALRDGNNWDYFNGMDVWGQGTTVTVSS

SEQ ID NO: 1495 - protein, 256 aa, Artificial Sequence
  Description of Artificial Sequence: Synthetic polypeptide
QSVLTQPASVSGSPGQSITISCTGTSSDVGGYNYVSWYQQHPGKAPKLMIYEVSNRPSGVSNRFSGSKSGNTASLTISGLQAEDEADYYCSSYTSSYTYVFGTGTKLTVLGGSEGKSSGSGSESKSTGGSEVQLVESGGDLVQPGGSLRLSCAASGFTFNNYNMNWVRQAPGKGLEWVSHISTSSSNKYYADSVKGRFSISRDIAKNSMYLQMNSLRDEDTAVYYCARDGVGADYGDYYYYGMDVWGQGTTVTVSS

SEQ ID NO: 1496 - protein, 250 aa, Artificial Sequence
  Description of Artificial Sequence: Synthetic polypeptide
QSVLTQPPSVSVAPGQTARITCGGNNSGSKSVHWYQQKPGQAPVLVVYDDSDRPSGIPERFSGSNSGNTATLTISRVEAGDEADYYCQVWDSSSDHGVFGGGTKLTVLGGSEGKSSGSGSESKSTGGSQVQLVESGGGEVQPGRSLRLSCAASGFSFSGYGMHWVRQAPGKGLEWVAVMSYDGSNRFYVDSVRGRFSISRDNSKNTLYLQMNSLRPEDTAVYYCARDTVWGSHPDAFDIWGQGTVVTVSS

SEQ ID NO: 1497 - protein, 247 aa, Artificial Sequence
  Description of Artificial Sequence: Synthetic polypeptide
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVLGGSEGKSSGSGSESKSTGGSQVQLVESGGGVVQPGRSLRLSCAASGFTFSSYNMNWVRQAPGKGLEWVAIIYYDGSNKYYADSVKGRFTISRDISKNTLYLQMNSLRAEDTAVYYCARERGRDYYGMDVWGQGTTVTVSS

SEQ ID NO: 1498 - protein, 248 aa, Artificial Sequence; identical to SEQ ID NO: 1490
  Description of Artificial Sequence: Synthetic polypeptide

SEQ ID NO: 1499 - protein, 254 aa, Artificial Sequence
  Description of Artificial Sequence: Synthetic polypeptide
EVQLVESGGGVVQPGRSLRLSCAASGFSFSGYGMHWVRQAPGKGLEWVAVISYDGSNKYYADSVKGRFTISRDNSKNTLYLQMNSLRVEDTAVYYCARDRIWGSRGYYYGMDVWGQGTTVTVSSGGSEGKSSGSGSESKSTGGSQSALTQPASVSGSPGQSITISCTGASSDVGGYNYVSWYQQHPGKAPKLMIYEVSNRPSGVSNRFSGSKSGNTASLTISGLQAEDEADYYCSSYTITSTLVFGGGTKLTVL

SEQ ID NO: 1500 - protein, 250 aa, Artificial Sequence
  Description of Artificial Sequence: Synthetic polypeptide
QVQLVESGGGEVQPGRSLRLSCAASGFSFSGYGMHWVRQAPGKGLEWVAVMSYDGSNRFYVDSVRGRFSISRDNSKNTLYLQMNSLRPEDTAVYYCARDTVWGSHPDAFDIWGQGTVVTVSSGGSEGKSSGSGSESKSTGGSQSVLTQPPSVSVAPGQTARITCGGNNSGSKSVHWYQQKPGQAPVLVVYDDSDRPSGIPERFSGSNSGNTATLTISRVEAGDEADYYCQVWDSSSDHGVFGGGTKLTVL

SEQ ID NO: 1501 - DNA, 1353 bp, Artificial Sequence
  Description of Artificial Sequence: Synthetic polynucleotide
gaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggcatcggctctacctactacgccgactctgtgaagggcagattcaccatcagccgggacaactccaagaacaccctgtggctgcagatgaactccctgagagccgaggataccgccgtgtactactgtgccaaggatgccgtgggcgctaccccttactacttcgattattggggccagggcaccctggtcaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 1502 - DNA, 1443 bp, Artificial Sequence
  Description of Artificial Sequence: Synthetic polynucleotide
gagatcgtgctgacccagtctcctggaactctgtctgtgtctcctggcgagagagctaccctgtcttgcagagcctctcagtccgtgcggtctaacctggcctggtatcagcagaaacctggacaggcccctagactgctgatctacggcgcttctaccagagccacaggcatccctgccagattttctggctctggctccggcaccgagttcaccctgacaatcagttccctgcagtccgaggacttcgccgtgtactactgccaccagtacaacgactggcctccttacacctttggccagggcaccaagctggaaatcaaaggcggctccgagggcaagagcagcggcagcggcagcgagagcaagagcaccggcggcagccaagttcagttgcaagaatctggtggcggcgtggtgcagcctggaagatctctgagactgtcttgtgccgcctccggcttcaccttctccacctatggaatgcattgggtccgacaggctcccggcaaaggactggaatgggtcgccttcatctcctacgacggctccaacaagtactacgccgactccgtgaagggcagattcaccatctctcgggacaactccaagcacaccctgtacctgcagatgaactccctgagagccgaggataccgctgtgtactattgcgccggcagagacaacctgcggttcctggaatggttcatggatgtgtggggccagggaaccaccgtgacagttagttctgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacgtgctgcccccatcccgggaggagatgaccaagaaccaggtcagcctgctgtgcctggtcaaaggcttctatcccagcgacatcgccgtggagtgggagagcaatgggcagccggagaacaactacctcacctggcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 1503 - DNA, 744 bp, Artificial Sequence
  Description of Artificial Sequence: Synthetic polynucleotide
cagtccgtgctgacccagcctccttctgcttctggaacacctggccagagagtgaccatctcctgctccggctcctcctccaacatcggctctaacaccgtgaactggtatcagcagctgcccggcacagcccctaaactgctgatctactcttccaaccagcggccttctggcgtgcccgatagattctctggctccaagtctggcacctccgctagcctggctatttctggcctgcagtctgaggacgaggccgattactactgtgccgcctgggatgattctctgaacggcgttgtgtttggcggaggcaccaaattgacagttcttggcggctccgagggcaagagcagcggcagcggcagcgagagcaagagcaccggcggcagcgaagttcagctgttggaatctggacctggcctggtcaagccttccgagacactgtctctgacctgtaccgtgtccggcggctccatcatctcctactactggtcctggatcagacagcctgccggcaaaggactggaatggatcggcagaatctactcctccggcagcaccaactacaaccccagcctgaagtcccgcgtgaccatgtctgtggacacctccaagaaccagttctccctgaagctgtcctctgtgaccgccgctgataccgctgtgtactactgcgctaaagtcggagtgtggcctggcgcctttgatatctggggacagggcacaatggtcaccgtgtcctct

SEQ ID NO: 1504 - protein, 479 aa, Artificial Sequence
  Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYNGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1505 - protein, 121 aa, Artificial Sequence
  Description of Artificial Sequence: Synthetic polypeptide
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 1506 - protein, 12 aa, Artificial Sequence
  Description of Artificial Sequence: Synthetic peptide
PQVHYDYAGFPY

SEQ ID NO: 1507 - protein, 11 aa, Artificial Sequence
  Description of Artificial Sequence: Synthetic peptide
PQVHYDYAGFP

SEQ ID NO: 1508 - protein, 10 aa, Artificial Sequence
  Description of Artificial Sequence: Synthetic peptide
GYTFTRSTMH

SEQ ID NO: 1509 - protein, 10 aa, Artificial Sequence
  Description of Artificial Sequence: Synthetic peptide
YINPSSAYTN

SEQ ID NO: 1510 - protein, 6 aa, Artificial Sequence
  Description of Artificial Sequence: Synthetic peptide
TRSTMH

SEQ ID NO: 1511 - protein, 13 aa, Artificial Sequence
  Description of Artificial Sequence: Synthetic peptide
WIGYINPSSAYTN

SEQ ID NO: 1512 - protein, 13 aa, Artificial Sequence
  Description of Artificial Sequence: Synthetic peptide
ASPQVHYDYNGFP

SEQ ID NO: 1513 - protein, 6 aa, Artificial Sequence
  Description of Artificial Sequence: Synthetic peptide
SYMNWY

SEQ ID NO: 1514 - protein, 10 aa, Artificial Sequence
  Description of Artificial Sequence: Synthetic peptide
RWIYDSSKLA

SEQ ID NO: 1515 - protein, 8 aa, Artificial Sequence
  Description of Artificial Sequence: Synthetic peptide
QQWSRNPP

SEQ ID NO: 1516 - protein, 6 aa, Artificial Sequence
  Description of Artificial Sequence: Synthetic peptide
TRSTMH

SEQ ID NO: 1517 - protein, 13 aa, Artificial Sequence
  Description of Artificial Sequence: Synthetic peptide
ASPQVHYDYAGFP

SEQ ID NO: 1518 - protein, 14 aa, Artificial Sequence
  Description of Artificial Sequence: Synthetic peptide
ASPQVHYDYAGFPY

SEQ ID NO: 1519 - DNA, 363 bp, Artificial Sequence
  Description of Artificial Sequence: Synthetic polynucleotide
caggttcagctggttcagtctggcgccgaagtgaagaaacctggctcctccgtcaaggtgtcctgcaaggcttccggctacacctttaccagatccaccatgcactgggtcaaacaggctccaggacaaggcttggagtggatcggctacatcaaccccagctccgcctacaccaactacaaccagaaattccagggcagagtcaccctcaccgccgacaagtctacctccaccgcctacatggaactgtccagcctgagatctgaggacaccgccgtgtactactgcgccagccctcaggtgcactacgactacaacggcttcccttattggggccagggcaccctggttaccgtttcttct

SEQ ID NO: 1520 - DNA, 318 bp, Artificial Sequence
  Description of Artificial Sequence: Synthetic polynucleotide
gagatcgtgctgacccagtctcctgccacactgtcagcctctccaggcgagagagtcaccctgtcctgctccgcttcctcctccgtgtcctacatgaactggtatcagcagaagcccggccaggctcctagacggtggatctacgactcctccaagctggcctctggcgtccctgcccgcttttccggctctggctctggcagagactataccctgaccatctccagcctggaacctgaggacttcgccgtgtactactgccagcagtggtctagaaaccctcctacctttggcggaggcaccaaggtggaaatcaag

SEQ ID NO: 1521 - DNA, 363 bp, Artificial Sequence
  Description of Artificial Sequence: Synthetic polynucleotide
caggttcaactggttcagtctggcgccgaagtgaagaaacctggctcctccgtcaaggtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggtccactacgactacgccggctttccttattggggccagggcacactggtcaccgtttcttct

SEQ ID NO: 1522 - DNA, 318 bp, Artificial Sequence; identical to SEQ ID NO: 1520
  Description of Artificial Sequence: Synthetic polynucleotide

SEQ ID NO: 1523 - protein, 247 aa, Artificial Sequence
  Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYNGFPYWGQGTLVTVSS

SEQ ID NO: 1524 - protein, 247 aa, Artificial Sequence
  Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYQGFPYWGQGTLVTVSS

SEQ ID NO: 1525 - DNA, 742 bp, Artificial Sequence
  Description of Artificial Sequence: Synthetic polynucleotide
gaaattgttttgacccaatcacctgccactctctctgcctctcctggtgagcgagttactttgtcatgtagcgcatcatcaagtgtatcttacatgaactggtaccaacaaaaacccggacaggcaccacgtcgttggatttatgacagtagcaagctcgcctccggggtacccgcaagattttccgggtcagggtctggcagggactataccctgacaatcagcagtctggaacctgaggactttgctgtgtattactgccaacagtggtctcgcaacccccctactttcgggggaggtacaaaggtagaaattaagggcggctccgagggcaagagcagcggcagcggcagcgagagcaagagcaccggcggcagccaagtgcaactcgtgcaaagcggggctgaagtgaagaagcctggatcaagcgtgaaggtcagttgcaaagcctctggatataccttcactcgatcaaccatgcactgggtcaagcaggccccagggcaagggctcgaatggataggatatattaacccaagttctgcctacactaactataatcagaagtttcaaggccgggtaacacttacagccgataagagtacctcaacagcatacatggaacttagttctttgcggagcgaggataccgctgtgtattactgcgcttcacctcaggttcactacgactacgctggatttccctattggggtcagggtacactggttacagtttcctctg

SEQ ID NO: 1526 - protein, 250 aa, Artificial Sequence
  Description of Artificial Sequence: Synthetic polypeptide
QVQLQESGGDVVQPGRSLRLSCAASGFSFSGYGLHWVRQAPGRGLEWVTLISYDGSNKYYADSVKGRFTISRDNSKNTLYLQMNSLRAEDTAVYYCAKTTVSDPYYYGMDVWGQGTTVTVSSGGSEGKSSGSGSESKSTGGSSYELTQPPSVSVAPGQTARITCGGNNIGSKSVHWYQQKPGQAPVLVVYDDSDRPSGIPERFSGTNSGNTATLTISRAEAGDEADYYCQVWDSSSDHVVFGGGTKLTVL

SEQ ID NO: 1527 - protein, 247 aa, Artificial Sequence; identical to SEQ ID NO: 1497
  Description of Artificial Sequence: Synthetic polypeptide

SEQ ID NO: 1528 - protein, 250 aa, Artificial Sequence; identical to SEQ ID NO: 1496
  Description of Artificial Sequence: Synthetic polypeptide

SEQ ID NO: 1529 - protein, 256 aa, Artificial Sequence; identical to SEQ ID NO: 1495
  Description of Artificial Sequence: Synthetic polypeptide

SEQ ID NO: 1530 - protein, 255 aa, Artificial Sequence; identical to SEQ ID NO: 1494
  Description of Artificial Sequence: Synthetic polypeptide

SEQ ID NO: 1531 - protein, 250 aa, Artificial Sequence; identical to SEQ ID NO: 1526
  Description of Artificial Sequence: Synthetic polypeptide

SEQ ID NO: 1532 - DNA, 363 bp, Artificial Sequence
  Description of Artificial Sequence: Synthetic polynucleotide
gaggtgcagctggttgaatctggcggaggattggttcagcctggcggttctctgagactgtcttgtgccgcttccggcttcaccttctccagctacgctatgtcctgggtccgacaggctcctggcaaaggactggaatgggtgtccgctatctctggcggcatcggctctacctactacgccgactctgtgaagggcagattcaccatcagccgggacaactccaagaacaccctgtggctgcagatgaactccctgagagccgaggataccgccgtgtactactgtgccaaggatgccgtgggcgctaccccttactacttcgattattggggccagggcaccctggtcaccgtttcttct

SEQ ID NO: 1533 - DNA, 330 bp, Artificial Sequence
  Description of Artificial Sequence: Synthetic polynucleotide
cagtctgtgctgacccagcctccttctgtgtctgctgctcctggccagaaagtgaccatctcctgctccggctcctcctccaacatcggcatcaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttctctgtccgccgttgtttttggcggtggcaccaagctgacagtgctg

SEQ ID NO: 1534 - DNA, 648 bp, Artificial Sequence
  Description of Artificial Sequence: Synthetic polynucleotide
cagtctgtgctgacccagcctccttctgtgtctgctgctcctggccagaaagtgaccatctcctgctccggctcctcctccaacatcggcatcaactacgtgtcctggtatcagcagctgcccggcacagctcccaaactgctgatctacgacaacaacaagcggcccagcggcatccctgacagattctccggatctaagtccggcacctctgctaccctgggcatcacaggattgcagacaggcgacgaggccgactactattgcggcacctgggactcttctctgtccgccgttgtttttggcggtggcaccaagctgacagtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca